(19) United States
(12) Patent Application Publication
(10) Pub. No.: US 2025/0084409 A1
(43) Pub. Date: Mar. 13, 2025

(54) COMPOSITIONS AND METHODS FOR CAPPING RNAS

(71) Applicant: Verve Therapeutics, Inc., Boston, MA (US)

(72) Inventors: Christopher Cheng, North Reading, MA (US); Kallanthottathil Rajeev, Wayland, MA (US); Caroline Reiss, Somerville, MA (US); Kui Wang, Wellesley, MA (US); Jay Leipheimer, Boston, MA (US)

(21) Appl. No.: 18/819,275

(22) Filed: Nov. 25, 2024

Related U.S. Application Data
(63) Continuation of application No. PCT/US2023/014183, filed on Feb. 28, 2023.
(60) Provisional application No. 63/315,323, filed on Mar. 1, 2022.
(60) Provisional application No. 63/485,753, filed on Feb. 17, 2023.

(51) Int. Cl. C12N 15/11; A61K 31/713; C12N 9/22; C12N 9/78

(52) U.S. Cl. CPC C12N 15/11; A61K 31/713; C12N 9/22; C12N 9/78; C07K 2319/09; C12N 2310/315; C12N 2830/50; C12Y 305/04002

(57) ABSTRACT

Provided herein are compositions and methods for preparation of 5′ end region-modified mRNAs. In particular, the instant disclosure relates to novel mRNA 5′ end region motifs and sequence initiators therefore together with assays that are capable of measuring the aspects of the functionality of those motifs and sequence initiators. Further provided herein are compositions and methods of treating conditions related to coronary disease.

### CROSS-REFERENCE

This application is a continuation of International Application No. PCT/US2023/014183, filed Feb. 28, 2023 which claims the benefit of U.S. Provisional Application Nos. 63/315,323, filed Mar. 1, 2022, and 63/485,753, filed Feb. 17, 2023, the contents of which are fully incorporated herein by reference.

### REFERENCE TO A SEQUENCE LISTING XML

This application contains a Sequence Listing which has been submitted electronically in XML format. The Sequence Listing XML is incorporated herein by reference. Said XML file, created on Nov. 12, 2024, is named 53989-719_601_SL.xml and is 716,697 bytes in size.

### FIELD OF THE DISCLOSURE

The instant disclosure relates to RNA and in particular the 5′ end-region of an mRNA molecule, including for example, novel mRNA 5′ end region motifs (or mRNA Caps) and initiators thereof.

### BACKGROUND

The 5′ end region of an mRNA can be an important structural and/or functional feature of eukaryotic mRNA molecules as it is capable of providing stability to the mRNA (e.g., by providing protection against 5′ exonucleases) and being involved in RNA splicing, mRNA transport, and other activities that support protein translation. A structural element of a conventional mRNA Cap frequently comprises an inverted 7-methylguanosine (m⁷G) linked at the 5′ end to a triphosphate (ppp) bridge, which phosphate bridge is in turn linked to the first nucleotide (N₁) of the mRNA transcript. The 5′ end region mRNA motifs and sequence initiators thereof described here differ from the conventional mRNA Caps in several respects, including, for example, that they comprise a chemically modified inverted 7-methylguanosine (m7G) nucleoside structure and/or modified triphosphate (ppp) linkage. Conventional Caps are often one of the most expensive components in the manufacture of mRNA molecules. Accordingly, alternatives to conventional Caps while maintaining or improving capping efficiencies and/or mRNA yields are each independently important aspects to the field.

### SUMMARY

Novel mRNA 5′ end region motifs and initiators therefore are described herein.

In one aspect, described herein is an in vitro-transcribed (IVT) mRNA sequence initiator comprising a compound of Formula (I) or a salt or solvate thereof.

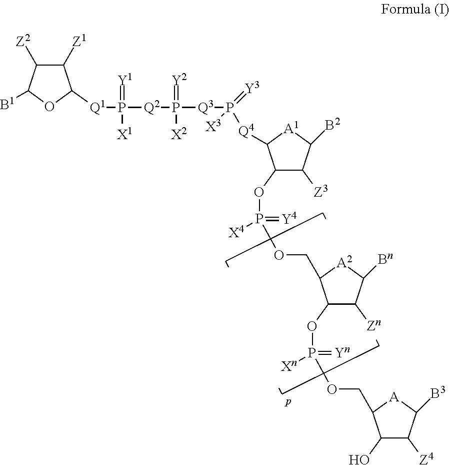

wherein
B1 is

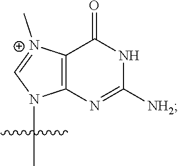 [SMILES: *n1c[n+](C)c2c(=O)[nH]c(N)nc21]

each B2, B3, and Bn is independently a natural, a modified, or an unnatural nucleobase;
each Z1 and Z2 is independently hydrogen, fluorine, —OH, —SH, —CH3, —CH2CH3, —OCH3, —OCH2CH3, —SCH3, —NH2, NHCH3, or NHC(═O)CH3;
each Z3, Z4, and Zn is independently hydrogen, fluorine, —OH, —SH, —CH3, —CH2CH3, —OCH3, —NH2, —NHCH3, —NH(C(═O)CH3), —OCH2CH3, —OCH2OCH3, —OCH2CH2CH3, —OCH(CH3)2, —SCH3, or —OCH2CH2OCH3;
each Q1 and Q4 is independently —CH2-, —CH═CH—, —CH2O—, —CH₂S—, —CH2CH2-, —CH2CF2-, —CH2NH2-, —CH2NH(CH3)-, or —CH2N(C(═O)CH3)-;
each Q2 and Q3 is independently —O—, —S—, —CH2-, —CF2-, —NH—, —N(CH3)-, or —N(C(═O)CH3)-;
each X1, X2, X3, X4, and Xn is independently —OH, —SH, —O⁻, —S⁻, —NH2, —NHCH3, —NH(C(═O)CH3), —CH3, —CH2CH3, —CH2CH2CH3, —CH(CH3)2, —OCH3, or —OCH2CH3;
each Y1, Y2, Y3, Y4, and Yn is independently ═O, ═S, ═NH, or ═NCH3;
each A, A1, and A2 is independently —O—, —S—, —CH2-, —NH—, —N(CH3)- or —N(C(═O)CH3)-; and
p=0, 1, 2, 3, 4, 5 or 6.

In another aspect, described herein the compound of Formula (I) satisfies one or more of the following proviso (i) to (iii): (i) at least one of X1, X2, X3, X4, and Xn is —SH or —S—; (ii) at least one of Y1, Y2, Y3, Y4, and Yn is ═S; and (iii) at least one of A, A1, and A2 is —S—.

In another aspect, the IVT mRNA sequence initiator satisfies at least one of X1, X2, X3, X4, and Xn is —SH or —S—; at least one of Y1, Y2, Y3, Y4, and Yn is ═S; or at least one of A, A1, and A2 is —S—.

In one aspect, described herein is an in vitro-transcribed (IVT) mRNA sequence initiator comprising a compound of Formula (II) or a salt or solvate thereof:

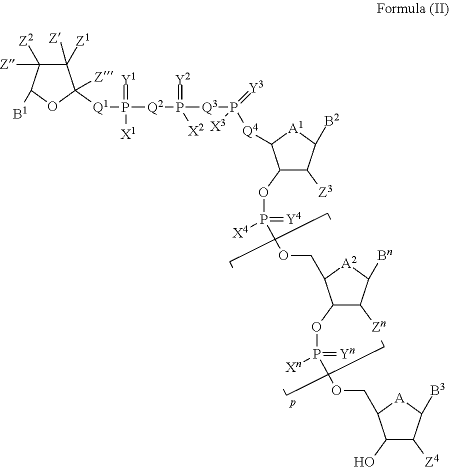

wherein
B1 is

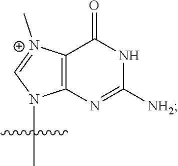 [SMILES: *n1c[n+](C)c2c(=O)[nH]c(N)nc21]

each B2, B3, and Bn is independently a natural, a modified, or an unnatural nucleobase;
each Z1 and Z′ is independently is hydrogen, fluorine, —OH, —SH, —CH3, —CH2CH3, —OCH3, —NH(CH3), —NH2, —NH(C(═O)CH3), or —SCH3;
each Z2 and Zn is independently fluorine, —OH, —SH, —CH3, —CH2CH3, —OCH3, —SCH3, —OCH2CH3, —NH2, NHCH3, or NHC(═O)CH3;
Z′″ is hydrogen, fluorine, —CH3, —CH2CH3, —OCH3, or —OCH2CH3;
each Z3, Z4, and Zn is independently hydrogen, fluorine, —OH, —CH3, —CH2CH3, —OCH3, —NH2, —NHCH3, —NH(C(═O)CH3), —OCH2CH3, —OCH2OCH3, —OCH2CH2CH3, —OCH(CH3)2, —SCH3, or —OCH2CH2OCH3;
each Q1 and Q4 is independently —CH═CH—, —CH2-, —CH2O—, —CH₂S—, —CH2CH2-, —CH2CF2-, —CH2NH2-, —CH2NH(CH3)-, or —CH2N(C(═O)CH3)-;
each Q2 and Q3 is independently —O—, —S—, —CH2-, —CF2-, —NH—, —N(CH3)-, or —N(C(═O)CH3)-;
each X1, X2, X3, X4, and Xn is independently —OH, —SH, —O⁻, —S⁻, —NH2, —NHCH3, —NH(C(═O)CH3), —CH3, —CH2CH3, —CH2CH2CH3, —CH(CH3)2, —OCH3 or —OCH2CH3;
each Y1, Y2, Y3, Y4, and Yn is independently ═O, ═S, ═NH, or ═NCH3;
each A, A1, and A2 is independently —O—, —S—, —CH2-, —NH—, —N(CH3)- or —N(C(═O)CH3)-; and
p=0, 1, 2, 3, 4, 5 or 6.

In one aspect, described herein is an in vitro-transcribed (IVT) mRNA sequence initiator comprising a compound of Formula (II) or a salt or solvate thereof:

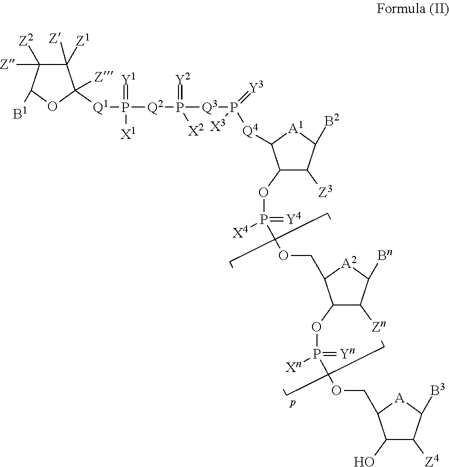

wherein
B1 is

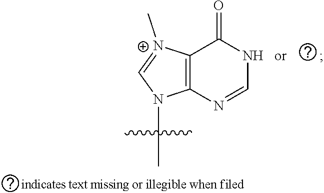

each B2, B3, and Bn is independently a natural, a modified, or an unnatural nucleobase;
each Z′ and Z″ is independently is hydrogen, fluorine, —OH, —SH, —CH3, —CH2CH3, —OCH3, —NH(CH3), —NH2, —NH(C(═O)CH3), or —SCH3;
Z′″ is hydrogen, fluorine, —CH3, —CH2CH3, —OCH3, or —OCH2CH3;
each Z1 and Z2 is independently hydrogen, fluorine, —OH, —SH, —CH3, —CH2CH3, —OCH3, —SCH3, —OCH2CH3, —NH2, NHCH3, or NHC(═O)CH3;
each Z3, Z4, and Zn is independently hydrogen, fluorine, —OH, —CH3, —CH2CH3, —OCH3, —NH2, —NHCH3, —NH(C(═O)CH3), —OCH2CH3, —OCH2OCH3, —OCH2CH2CH3, —OCH(CH3)2, —SCH3, or —OCH2CH2OCH3;
each Q1 and Q4 is independently —CH═CH—, —CH2-, —CH2O—, —CH₂S—, —CH2CH2-, —CH2CF2-, —CH2NH2-, —CH2NH(CH3)-, or —CH2N(C(═O)CH3)-;
each Q2 and Q3 is independently —O—, —S—, —CH2-, —CF2-, —NH—, —N(CH3)-, or —N(C(═O)CH3)-;
each X1, X2, X3, X4, and Xn is independently —OH, —SH, —O⁻, —S⁻, —NH2, —NHCH3, —NH(C(═O)CH3), —CH3, —CH2CH3, —CH2CH2CH3, —CH(CH3)2, —OCH3 or —OCH2CH3;
each Y1, Y2, Y3, Y4, and Yn is independently ═O, ═S, ═NH, or ═NCH3;
each A, A1, and A2 is independently —O—, —S—, —CH2-, —NH—, —N(CH3)- or —N(C(═O)CH3)-; and
p=0, 1, 2, 3, 4, 5 or 6.

In one aspect, described herein an in vitro-transcribed (IVT) mRNA sequence initiator comprising a compound of Formula (II) or a salt or solvate thereof.

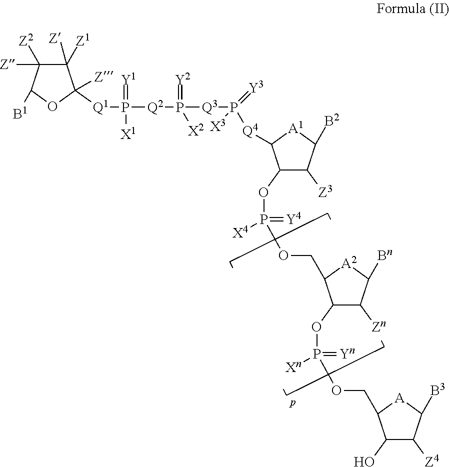

wherein
B¹ is

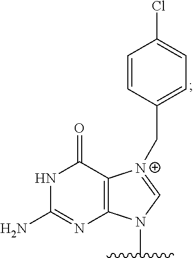 [SMILES: *n1c[n+](Cc2ccc(Cl)cc2)c2c(=O)[nH]c(N)nc21]

each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

In one aspect, described herein is an in vitro-transcribed (IVT) mRNA sequence initiator comprising a compound of Formula (III) or a salt or solvate thereof:

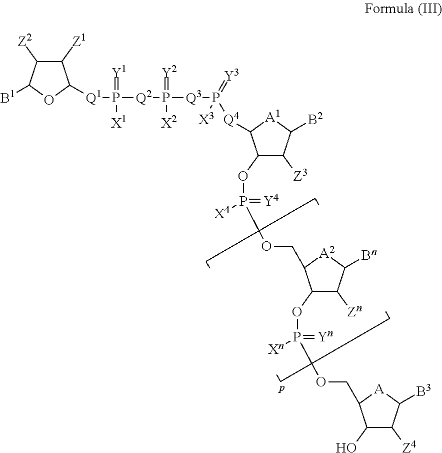

wherein
B1 is

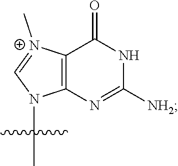 [SMILES: *n1c[n+](C)c2c(=O)[nH]c(N)nc21]

B2 is a modified or an unnatural nucleobase;
each B3, and Bn is independently a natural, a modified, or an unnatural nucleobase;
each Z1 and Z2 is independently hydrogen, fluorine, —OH, —SH, —CH3, —CH2CH3, —OCH3, —OCH2CH3, —SCH3, —NH2, NHCH3, or NHC(═O)CH3;
each Z3, Z4, and Zn is independently hydrogen, fluorine, —OH, —SH, —CH3, —CH2CH3, —OCH3, —NH2, —NHCH3, —NH(C(═O)CH3), —OCH2CH3, —OCH2OCH3, —OCH2CH2CH3, —OCH(CH3)2, —SCH3, or —OCH2CH2OCH3;
each Q1 and Q4 is independently —CH2-, —CH═CH—, —CH2O—, —CH2S—, —CH2CH2-, —CH2CF2-, —CH2NH2-, —CH2NH(CH3)-, or —CH2N(C(═O)CH3)-;
each Q2 and Q3 is independently —O—, —S—, —CH₂—, —CF2-, —NH—, —N(CH₃)—, or —N(C(═O)CH₃)—;
each X1, X2, X3, X4, and Xn is independently —OH, —SH, —O⁻, —S⁻, —NH2, —NHCH3, —NH(C(═O)CH₃), —CH₃, —CH₂CH₃, —CH₂CH₂CH₃, —CH(CH₃)₂, —OCH3, or —OCH2CH₃;
each Y1, Y2, Y3, Y4, and Yⁿ is independently ═O, ═S, ═NH, or ═NCH3;
each A, A1, and A2 is independently —O—, —S—, —CH₂—, —NH—, —N(CH₃)— or —N(C(═O)CH₃)—; and p=0, 1, 2, 3, 4, 5 or 6.

In another aspect, Z³ is hydrogen, fluorine, —OH, —OCH3, - or —OCH2CH₃. In another aspect, wherein Z³ is —OCH3. In another aspect, Z⁴ and Zn are independently —OH or —OCH3. In another aspect, each Z3, Z4, and Zn are independently —OH or —OCH3. In another aspect, Y2, Y4, and Yⁿ are independently ═O or ═S. In another aspect, X2 and X3 are independently —O— or —S—.

In some embodiments, a compound of Formula (III) is a salt. In some embodiments, the salt is an alkali metal salt. In some embodiments, the salt is a sodium salt. In some embodiments, the salt is an ammonium salt.

In another aspect, the IVT mRNA sequence initiator comprises a phosphorothioate. In another aspect, the phosphorothioate comprises a chiral phosphorous center. In another aspect, described herein is a composition comprising the IVT mRNA sequence initiator, wherein the composition comprises a racemic mixture of a R diastereomer and a S diastereomer of the IVT mRNA sequence initiator. In another aspect, a stereochemical purity of the IVT mRNA sequence initiator in the composition is at least 60%. In another aspect, a stereochemical purity of the IVT mRNA sequence initiator in the composition is at least 80%. In another aspect, a stereochemical purity of the IVT mRNA sequence initiator in the composition is at least 90%. In another aspect, a stereochemical purity of the IVT mRNA sequence initiator in the composition is at least 95%. In another aspect, a stereochemical purity of the IVT mRNA sequence initiator in the composition is at least 98%.

In one aspect, described herein is an mRNA sequence having a 5′-end region motif (I′):

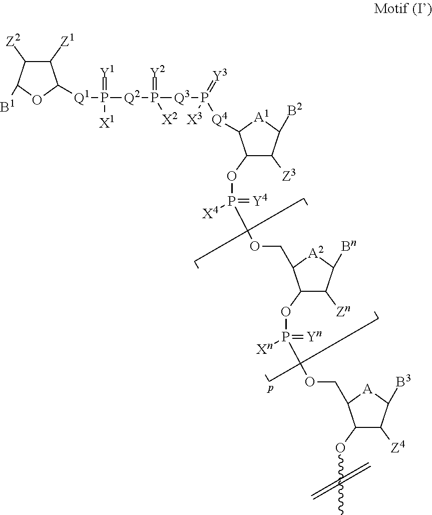

wherein
B1 is

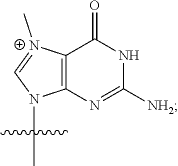 [SMILES: *n1c[n+](C)c2c(=O)[nH]c(N)nc21]

each B2, B3, and Bn is independently a natural, a modified, or an unnatural nucleobase;
each Z1 and Z2 is independently hydrogen, fluorine, —OH, —SH, —CH3, —CH2CH3, —OCH3, —OCH2CH3, —SCH3, —NH2, NHCH3, or NHC(═O)CH3;
each Z3, Z4, and Zn is independently hydrogen, fluorine, —OH, —SH, —CH3, —CH2CH3, —OCH3, —NH2, —NHCH3, —NH(C(═O)CH3), —OCH2CH3, —OCH2OCH3, —OCH2CH2CH3, —OCH(CH3)2, —SCH3, or —OCH2CH2OCH3;
each Q1 and Q4 is independently —CH2-, —CH═CH—, —CH2O—, —CH2S—, —CH2CH2-, —CH2CF2-, —CH2NH2-, —CH2NH(CH3)-, or —CH2N(C(═O)CH3)-;
each Q2 and Q3 is independently —O—, —S—, —CH2-, —CF2-, —NH—, —N(CH3)-, or —N(C(═O)CH3)-;
each X1, X2, X3, X4, and Xn is independently —OH, —SH, —O⁻, —S⁻, —NH2, —NHCH3, —NH(C(═O)CH3), —CH3, —CH2CH3, —CH2CH2CH3, —CH(CH3)2, —OCH3, or —OCH2CH3;
each Y1, Y2, Y3, Y4, and Yn is independently ═O, ═S, ═NH, or ═NCH3;
each A, A1, and A2 is independently —O—, —S—, —CH2-, —NH—, —N(CH3)- or —N(C(═O)CH3)-; and
p=0, 1, 2, 3, 4, 5 or 6.

In another aspect, Formula (I) satisfies one or more of the following proviso (i) to (iii): (i) at least one of X1, X2, X3, X4, and Xn is —SH or —S—; (ii) at least one of Y1, Y2, Y3, Y4, and Yn is ═S; and (iii) at least one of A, A1, and A2 is —S—. In another aspect, at least one of X1, X2, X3, X4, and Xn is —SH or —S—; at least one of Y1, Y2, Y3, Y4, and Yn is ═S; or at least one of A, A1, and A2 is —S—.

In one aspect, described herein, an mRNA sequence having a 5′end region motif (II′):

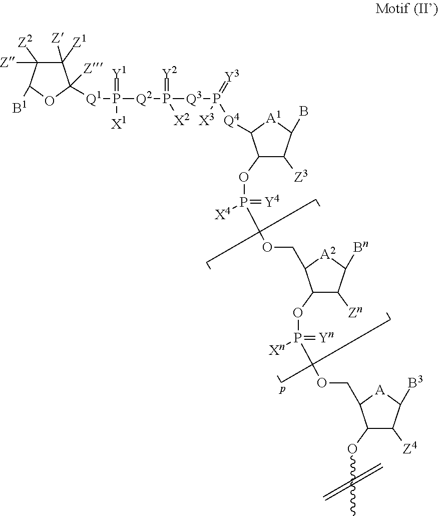

wherein
B1 is

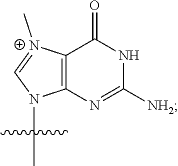 [SMILES: *n1c[n+](C)c2c(=O)[nH]c(N)nc21]

each B2, B3, and Bn is independently a natural, a modified, or an unnatural nucleobase;
each Z1 and Z′ is independently is hydrogen, fluorine, —OH, —SH, —CH3, —CH2CH3, —OCH3, —NH(CH3), —NH2, —NH(C(═O)CH3), or —SCH3;
each Z2 and Zn is independently fluorine, —OH, —SH, —CH3, —CH2CH3, —OCH3, —SCH3, —OCH2CH3, —NH2, NHCH3, or NHC(═O)CH3;
Z′″ is hydrogen, fluorine, —CH3, —CH2CH3, —OCH3, or —OCH2CH3;
each Z3, Z4, and Zn is independently hydrogen, fluorine, —OH, —CH3, —CH2CH3, —OCH3, —NH2, —NHCH3, —NH(C(═O)CH3), —OCH2CH3, —OCH2OCH3, —OCH2CH2CH3, —OCH(CH3)2, —SCH3, or —OCH2CH2OCH3;
each Q1 and Q4 is independently —CH═CH—, —CH2-, —CH2O—, —CH2S—, —CH2CH2-, —CH2CF2-, —CH2NH2-, —CH2NH(CH3)-, or —CH2N(C(═O)CH3)-;
each Q2 and Q3 is independently —O—, —S—, —CH2-, —CF2-, —NH—, —N(CH3)-, or —N(C(═O)CH3)-;
each X1, X2, X3, X4, and Xn is independently —OH, —SH, —O⁻, —S⁻, —NH2, —NHCH3, —NH(C(═O)CH3), —CH3, —CH2CH3, —CH2CH2CH3, —CH(CH3)2, —OCH3 or —OCH2CH3;
each Y1, Y2, Y3, Y4, and Yⁿ is independently ═O, ═S, ═NH, or ═NCH3;
each A, A1, and A2 is independently —O—, —S—, —CH2-, —NH—, —N(CH₃)— or —N(C(═O)CH₃)—; and
p=0, 1, 2, 3, 4, 5 or 6.

In one aspect, described herein, an mRNA sequence having a 5′-end region motif (II′):

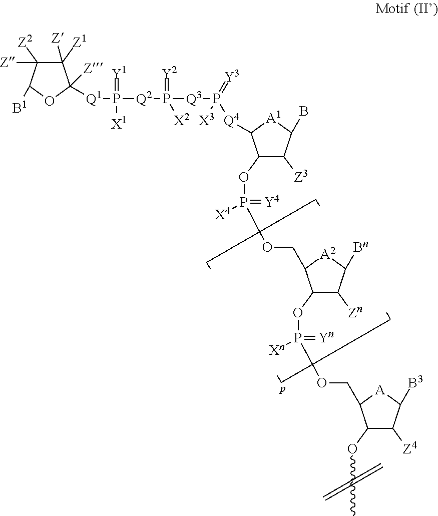

wherein
B1 is

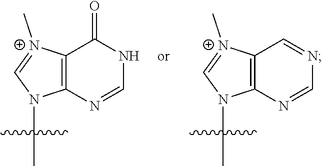 [SMILES: *n1c[n+](C)c2c(=O)[nH]cnc21 ; *n1c[n+](C)c2cncnc21]

each B2, B3, and Bn is independently a natural, a modified, or an unnatural nucleobase;
each Z′ and Z″ is independently is hydrogen, fluorine, —OH, —SH, —CH3, —CH2CH3, —OCH3, —NH(CH3), —NH2, —NH(C(═O)CH3), or —SCH3; Z′″ is hydrogen, fluorine, —CH3, —CH2CH3, —OCH3, or —OCH2CH3;
each Z¹ and Z² is independently hydrogen, fluorine, —OH, —SH, —CH3, —CH2CH3, —OCH3, —SCH3, —OCH2CH3, —NH2, NHCH3, or NHC(═O)CH3;
each Z3, Z4, and Zn is independently hydrogen, fluorine, —OH, —CH3, —CH2CH3, —OCH3, —NH2, —NHCH3, —NH(C(═O)CH3), —OCH2CH3, —OCH2OCH3, —OCH2CH2CH3, —OCH(CH3)2, —SCH3, or —OCH2CH2OCH3;
each Q1 and Q4 is independently —CH═CH—, —CH2-, —CH2O—, —CH2S—, —CH2CH2-, —CH2CF2-, —CH2NH2-, —CH2NH(CH3)-, or —CH2N(C(═O)CH3)-;
each Q2 and Q3 is independently —O—, —S—, —CH2-, —CF2-, —NH—, —N(CH3)-, or —N(C(═O)CH3)-;
each X1, X2, X3, X4, and Xn is independently —OH, —SH, —O⁻, —S⁻, —NH2, —NHCH3, —NH(C(═O)CH3), —CH3, —CH2CH3, —CH2CH2CH3, —CH(CH3)2, —OCH3 or —OCH2CH3;
each Y1, Y2, Y3, Y4, and Yn is independently ═O, ═S, ═NH, or ═NCH3;
each A, A1, and A2 is independently —O—, —S—, —CH2-, —NH—, —N(CH3)- or —N(C(═O)CH3)-; and
p=0, 1, 2, 3, 4, 5 or 6.

In one aspect, described herein is an mRNA sequence having a 5′-end region motif (II′):

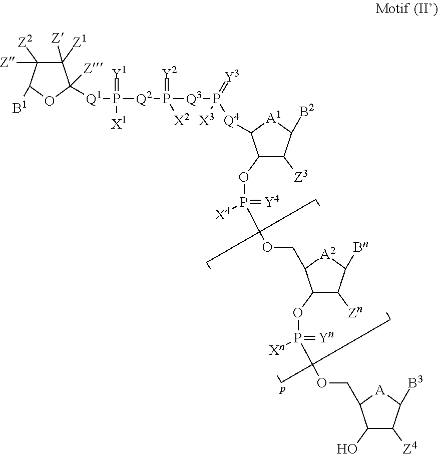

wherein
B¹ is

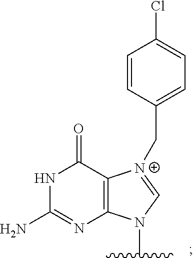 [SMILES: *n1c[n+](Cc2ccc(Cl)cc2)c2c(=O)[nH]c(N)nc21]

each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

In one aspect, described herein is an mRNA sequence having a 5′-end region motif (III′):

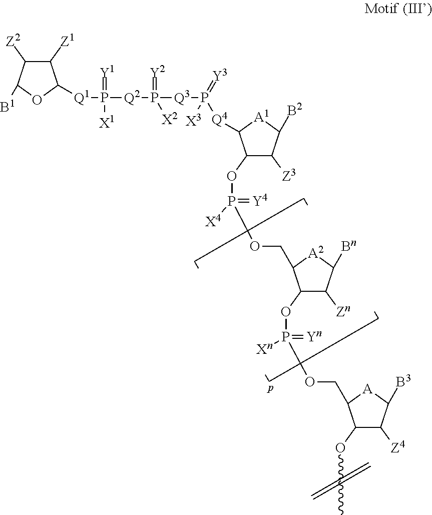

wherein
B1 is

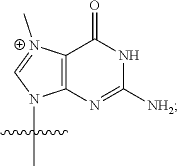 [SMILES: *n1c[n+](C)c2c(=O)[nH]c(N)nc21]

B2 is a modified or an unnatural nucleobase;
each B3, and Bⁿ is independently a natural, a modified, or an unnatural nucleobase;
each Z1 and Z2 is independently hydrogen, fluorine, —OH, —SH, —CH3, —CH2CH3, —OCH3, —OCH2CH3, —SCH3, —NH2, NHCH3, or NHC(═O)CH3;
each Z3, Z4, and Zn is independently hydrogen, fluorine, —OH, —SH, —CH3, —CH2CH3, —OCH3, —NH2, —NHCH3, —NH(C(═O)CH3), —OCH2CH3, —OCH2OCH3, —OCH2CH2CH3, —OCH(CH3)2, —SCH3, or —OCH2CH2OCH3;
each Q1 and Q4 is independently —CH2-, —CH═CH—, —CH2O—, —CH2S—, —CH2CH2-, —CH2CF2-, —CH2NH2-, —CH2NH(CH3)-, or —CH2N(C(═O)CH3)-;
each Q2 and Q3 is independently —O—, —S—, —CH2-, —CF2-, —NH—, —N(CH3)-, or —N(C(═O)CH3)-;
each X1, X2, X3, X4, and Xn is independently —OH, —SH, —O⁻, —S⁻, —NH2, —NHCH3, —NH(C(═O)CH3), —CH3, —CH2CH3, —CH2CH2CH3, —CH(CH3)2, —OCH3, or —OCH2CH3;
each Y1, Y2, Y3, Y4, and Yn is independently ═O, ═S, ═NH, or ═NCH3;
each A, A1, and A2 is independently —O—, —S—, —CH₂—, —NH—, —N(CH₃)— or —N(C(═O)CH₃)—; and
p=0, 1, 2, 3, 4, 5 or 6.

In one aspect, described herein is a mRNA sequence having a 5′-end region motif, wherein the 5′-end region motif is a compound from Table 1, or a salt or solvate thereof.

In another aspect, described herein is a mRNA sequence having a 5′-end region motif, wherein the 5′-end region motif is a compound from Table 2, or a salt or solvate thereof.

In another aspect, described herein is a mRNA sequence having a 5′-end region motif, wherein the 5′-end region motif is a compound from Table 3, or a salt or solvate thereof.

In another aspect, the mRNA sequence initiator is a salt. In some embodiments, the mRNA sequence initiator is a sodium salt.

In another aspect of the mRNA sequence having a 5′-end region motif, Z³ is hydrogen, fluorine, —OH, —OCH₃, - or —OCH₂CH₃.

In another aspect of the mRNA sequence having a 5′-end region, Z³ is —OCH₃.

In another aspect of the mRNA sequence having a 5′-end region motif, each Z⁴ and Zⁿ is independently —OH or —OCH₃.

In another aspect of the mRNA sequence having a 5′-end region motif, each Z³, Z⁴, and Zⁿ is independently —OH or —OCH₃.

In another aspect of the mRNA sequence having a 5′-end region motif, B¹ is

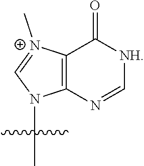 [SMILES: *n1c[n+](C)c2c(=O)[nH]cnc21]

In another aspect of the mRNA sequence having a 5′-end region motif, B¹ is

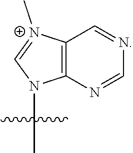 [SMILES: *n1c[n+](C)c2cncnc21]

In another aspect of the mRNA sequence having a 5′-end region motif, B¹ is

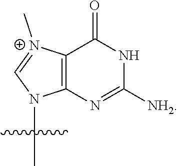 [SMILES: *n1c[n+](C)c2c(=O)[nH]c(N)nc21]

In another aspect of the mRNA sequence having a 5′-end region motif, B¹ is

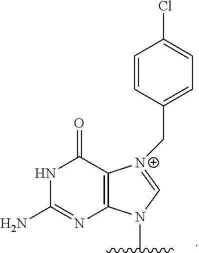 [SMILES: *n1c[n+](Cc2ccc(Cl)cc2)c2c(=O)[nH]c(N)nc21]

In another aspect of the mRNA sequence having a 5′-end region motif, B² is

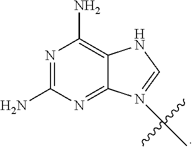

In another aspect of the mRNA sequence having a 5′-end region motif, Z¹ is fluorine, —OH, or —OCH₃.

In another aspect of the mRNA sequence having a 5′-end region motif, Z¹ is fluorine.

In another aspect of the mRNA sequence having a 5′-end region motif, Z¹ is —OH.

In another aspect of the mRNA sequence having a 5′-end region motif, Z¹ is —OCH₃.

In another aspect of the mRNA sequence having a 5′-end region motif, Z² is fluorine, —OH, or —OCH₃.

In another aspect of the mRNA sequence having a 5′-end region motif, Z² is fluorine.

In another aspect of the mRNA sequence having a 5′-end region motif, Z² is —OH.

In another aspect of the mRNA sequence having a 5′-end region motif, Z² is —OCH₃.

In another aspect of the mRNA sequence having a 5′-end region motif, Q¹ and Q⁴ is —CH₂O—.

In another aspect of the mRNA sequence having a 5′-end region motif, each Q² and Q³ is —O—.

In another aspect of the mRNA sequence having a 5′-end region motif, each Y¹ and Y³ is ═O.

In another aspect of the mRNA sequence having a 5′-end region motif, each Y², Y⁴, and Yⁿ is independently ═O or ═S.

In another aspect of the mRNA sequence having a 5′-end region motif, one or more of Y, Y², Y³, Y⁴, and Yⁿ is ═S.

In another aspect of the mRNA sequence having a 5′-end region motif, Y² is ═S.

In another aspect of the mRNA sequence having a 5′-end region motif, Y⁴ is ═S.

In another aspect of the mRNA sequence having a 5′-end region motif, each Y¹, Y2 Y³, Y⁴, and Yⁿ is ═O.

In another aspect of the mRNA sequence having a 5′-end region motif, each X¹, X⁴, and Xⁿ is —O—.

In another aspect of the mRNA sequence having a 5′-end region motif, each X² and X³ is independently —O— or —S—.

In another aspect of the mRNA sequence having a 5′-end region motif, X³ is —O—.

In another aspect of the mRNA sequence having a 5′-end region motif, one or more of X¹, X², X³, X⁴, and Xⁿ is —S—.

In another aspect of the mRNA sequence having a 5′-end region motif, X² is —S—.

In another aspect of the mRNA sequence having a 5′-end region motif, X⁴ is —S—.

In another aspect of the mRNA sequence having a 5′-end region motif, each X¹, X², X³, X⁴, and Xⁿ is —O—.

In another aspect of the mRNA sequence having a 5′-end region motif, each A, A¹, and A² is —O—.

In another aspect of the mRNA sequence having a 5′-end region motif, one or more of A, A¹, and A² is —S—.

In another aspect of the mRNA sequence having a 5′-end region motif, A is —S— and A¹ and A² is —O—.

In another aspect of the mRNA sequence having a 5′-end region motif, A² is —S— and A and A¹ is —O—.

In another aspect of the mRNA sequence having a 5′-end region motif, A, A¹, and A² is —O—.

In another aspect of the mRNA sequence having a 5′-end region motif, p is 0.

In another aspect of the mRNA sequence having a 5′-end region motif, p is 1.

In another aspect of the mRNA sequence having a 5′-end region motif, p is 2.

In another aspect of the e mRNA sequence having a 5′-end region motif, wherein each B², B³, and Bⁿ is independently adenine, cytosine, guanine, uracil, thymine, hypoxanthine, or purine.

In another aspect of the mRNA sequence having a 5′-end region motif, B² is adenine and B³ is guanine.

In another aspect of the mRNA sequence having a 5′-end region motif, B² is guanine and B³ is adenine.

In another aspect of the mRNA sequence having a 5′-end region motif, protein expression is increased.

In another aspect of the mRNA sequence having a 5′-end region motif, Q¹ and Q⁴ are —CH₂O—; Q² and Q³ are —O—; each Xⁿ is independently —OH, —SH, O⁻, or S⁻; each Yⁿ is independently ═O or ═S; and B¹ is

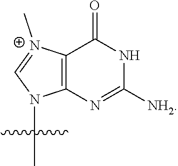 [SMILES: *n1c[n+](C)c2c(=O)[nH]c(N)nc21]

In another aspect of the mRNA sequence having a 5′-end region motif, Q¹ and Q⁴ are —CH₂O—; Q² and Q³ are —O—; each Xⁿ is independently —OH, —SH, O⁻, or S⁻; each Yⁿ is independently ═O or ═S; and B¹ is

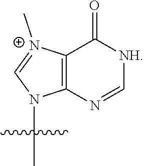 [SMILES: *n1c[n+](C)c2c(=O)[nH]cnc21]

In another aspect of the mRNA sequence having a 5′-end region motif, Q¹ and Q⁴ are —CH₂O—; Q² and Q³ are —O—; each Xⁿ is independently —OH, —SH, O⁻, or S⁻; each Yⁿ is independently ═O or ═S; and B¹ is

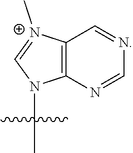 [SMILES: *n1c[n+](C)c2cncnc21]

In another aspect, described herein is a complex comprising an mRNA sequence having a 5′end region motif and a DNA template, wherein the mRNA sequence having a 5′-end region motif comprises a compound described herein, wherein the DNA template comprises a promoter region comprising a transcriptional start site having a first nucleotide at nucleotide position +1, a second nucleotide at nucleotide position +2, and a third nucleotide at nucleotide position +3; and wherein the mRNA sequence having a 5′-end region motif is hybridized to the DNA template at least at nucleotide positions +1, +2, and +3.

In another aspect, described herein is a complex comprising an mRNA sequence having a 5′-end region motif and a DNA template, wherein the mRNA sequence having a 5′end region motif comprises a compound described herein, wherein the DNA template comprises a promoter region comprising a transcriptional start site having a first nucleotide at nucleotide position +1 and a second nucleotide at nucleotide position +2; and wherein the mRNA sequence having a 5′-end region motif is hybridized to the DNA template at least at nucleotide positions +1 and +2.

In another aspect, described herein is a RNA molecule comprising the mRNA sequence having a 5′-end region motif. In another aspect, the RNA comprises a guide RNA or a nuclease mRNA. In another aspect, the RNA comprises an mRNA.

In another aspect, described herein is a method of expressing an mRNA comprising introducing the mRNA described herein into a cell lysate to express the mRNA. In another aspect, the method further comprises measuring the expression level of the mRNA. In another aspect, described herein in the method, the expression level of the mRNA is at least 2-fold, 3-fold, 4-fold, 6-fold, 8-fold, or 20-fold greater compared to a corresponding mRNA without the mRNA sequence having a 5′-end region motif. In another aspect, described herein, the method involves a HeLa cell. In another aspect, described herein the method involves producing at most 0.1 wt % dsRNA. In another aspect, described herein the method involves producing at most 0.09 wt % dsRNA. In another aspect, described herein the method involves producing at most 0.06 wt % dsRNA. In another aspect, described herein the method involves producing at most 0.04 wt % dsRNA.

In another aspect, described herein is a method of detecting cellular immune stimulation resulting from mRNA comprising (a) contacting a formulation comprising capped mRNA according the compounds described herein with a cell reporter line and (b) measuring RIG-I activation in said cell reporter line. In another aspect, the reporter line is HEK-Lucia RIG-I model. In another aspect, described herein the cellular immune stimulation is reduced compared to an uncapped mRNA by at least 20%, 50%, 70%, 100%, and 150%.

In another aspect, described herein is a method of producing an mRNA sequence having a 5′-end region motif described herein using an IVT reaction comprising (a) mixing a DNA template, polymerase enzyme, mRNA sequence motif comprising a phosphorothioate group (PS), and nucleoside triphosphates (NTPs) at a specified molar ratio of said mRNA sequence motif to said NTP to generate a mixture (b) incubating said mixture at a specified temperature and duration and (c) harvesinting and purifying said mRNA sequence having a 5′-end region motif from said mixture. In another aspect, the molar ratio is 1:5, and the method is capable of producing a yield of at least 80% with a capping efficiency of at least 80%. In another aspect, the molar ratio is 1:2.5, and the method is capable of producing a yield of at least 80% with a capping efficiency of at least 85%. In another aspect, the molar ratio is 1:1.67, and the method is capable of producing a yield of at least 80% with a capping efficiency of at least 90%. In another aspect, the molar ratio is 1:1.25, and the method is capable of producing a yield of at least 80% with a capping efficiency of at least 90%. In another aspect, the molar ratio is 1.0:1.0, and the method is capable of producing a yield of at least 80% with a capping efficiency of at least 80%. In another aspect, the molar ratio is 1:5, and the method is capable of producing a yield of at least 3 mg of mRNA per milliliter (mL) of IVT reaction with a capping efficiency of at least 80%. In another aspect, the molar ratio is 1:2.5, and the method is capable of producing a yield of at least 3 mg of mRNA per milliliter (mL) of IVT reaction with a capping efficiency of at least 85%. In another aspect, the molar ratio is 1:1.67, and the method is capable of producing a yield of at least 3 mg of mRNA per milliliter (mL) of IVT reaction with a capping efficiency of at least 90%. In another aspect, the molar ratio is 1:1.25, and the method is capable of producing a yield of at least 3 mg of mRNA per milliliter (mL) of IVT reaction with a capping efficiency of at least 90%. In another aspect, the molar ratio is 1.0:1.0, and the method is capable of producing a yield of at least 3 mg of mRNA per milliliter (mL) of IVT reaction with a capping efficiency of at least 80%. In another aspect, the NTP is GTP, ATP, CTP, UTP, a modified NTP, or a combination thereof. In another aspect, the modified NTP is N1-methyl pseudoridine.

In another aspect, described herein is cell containing an RNA molecule comprising the mRNA sequence having a 5′end region motif described herein.

In another aspect, described herein is a cell containing a polypeptide translated from an RNA molecule comprising the mRNA sequence having a 5′end region motif described herein.

In another aspect, described herein is a pharmaceutical composition comprising an RNA molecule comprising the mRNA sequence having a 5′end region motif described herein and one or more of pharmaceutically acceptable excipients. In another aspect, the pharmaceutical comprises lipid nanoparticles. In another aspect, the pharmaceutical composition is encapsulated in a-lipid nanoparticle. In another aspect, the pharmaceutical composition further comprises one or more single guide RNAs designed to target one or more specific locations of one or more genes of interest to elicit pharmacological effect upon administration into a mammal.

In another aspect, described herein is a method for synthesizing an RNA molecule comprising: introducing the mRNA sequence having a 5′end region motif described herein into a mixture comprising an RNA polymerase, and incubating the mixture for a time sufficient to allow for transcription of the RNA molecule. In another aspect, the mixture further comprises a DNA template and nucleoside triophosphates.

In another aspect, described herein is a method of gene editing comprising introducing into a cell an RNA molecule, or pharmaceutical composition, wherein the RNA molecule comprises guide RNA or a nuclease mRNA, wherein the RNA molecule is translated in the cell.

In another aspect, described herein is a method for reducing the risk of coronary disease in a subject in need thereof, comprising administering to the subject an effective amount of a pharmaceutical composition described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the invention are set forth with particularity in the appended claims and embodied in the mRNA 5′ end-region structures described herein. A better understanding of the features and advantages of the present invention will be obtained by reference to the detailed description that sets forth illustrative embodiments of the 5′ end-region structures or compounds (sometimes referred herein as motifs) in which the principles of the inventions are utilized, and the accompany drawings of which:

FIG. 1a shows phosophorothioate-modified 5′ end initiators and the corresponding in vitro transcribed mRNAs with 5′ end region motifs identified in the specification as 1007a.

FIG. 1b shows phosophorothioate-modified 5′ end initiators and the corresponding in vitro transcribed mRNAs with 5′ end region motifs identified in the specification as 1107a.

FIG. 2 illustrates a comparison of production yields for mRNAs that were prepared using different 5′ end initiators.

FIG. 3 illustrates a comparison of mRNA full-length purity between mRNAs that were prepared using different 5′ end initiators.

FIG. 4 illustrates a comparison of mRNA capping efficiency between mRNAs that were prepared using different 5′ end initiators.

FIG. 5 illustrates protein expression of mRNAs comprising different 5′ end region motifs in HeLa cell lysate.

FIG. 6 illustrates protein expression of mRNAs comprising different 5′ end region motifs in primary human hepatocyte cells.

FIG. 7 illustrates a comparison of ABE base editing using the protein encoded by mRNAs comprising different 5′ end-region motifs in vivo in mouse.

FIG. 8 illustrates a comparison of ABE base editing using protein encoded by two ABE mRNAs comprising different 5′ end region motifs in vivo in NHP.

FIG. 9 illustrates immune stimulation of mRNA comprising different 5′ end-regions motifs in HEK293 cells.

FIG. 10 illustrates the chirality of the phosphorothioate in 5′ initiator 1007a. 1007a_d1 represents the S diastereomer, while 1007a_d2 represents the R diastereomer.

FIG. 11a illustrates the efficiency of analog incorporation for 5′ initiator 5232 in comparison to analog 1002a as determined by ion-pairing reversed-phase high-performance liquid chromatography (IP-RP-HPLC) at doses ranging from 0.125 mM to 2 mM.

FIG. 11b shows the efficiency of analog incorporation for 5′ initiator 5232 in comparison to analog 1002a as determined by IP-RP-HPLC-mass spectrometry at doses of 0.5, 2 and 4 mM.

FIG. 12 shows the yield and quality of mRNA made using various 5′ initiator analogs.

FIG. 13 shows the percent dsRNA detected within mRNA samples produced using 5′ initiator analogs.

FIG. 14 illustrates in vitro RIG-I response to mRNA with various 5′ initiator caps.

FIG. 15 illustrates in vitro stability as a measurement of relative pyrophosphatase resistance for various 5′ initiator caps.

FIG. 16 illustrates in vitro 5′ initiator cap affinity for protein biogenesis factor through measuring relative ribosome affinity.

FIG. 17a illustrates temporal and dose-dependent expression of luciferase from mRNA synthesized with various 5′ initiator caps, where cells are harvested 6 hours after exposure to mRNA.

FIG. 17b illustrates temporal and dose-dependent expression of luciferase from RNA synthesized with various 5′ initiator caps, where cells are harvested 24 hours after exposure to mRNA.

FIG. 18a illustrates temporal and dose-dependent expression of Cas9 from mRNA synthesized with various 5′ initiator caps, where cells are harvested 6 hours after exposure to mRNA.

FIG. 18b illustrates temporal and dose-dependent expression of Cas9 from mRNA synthesized with various 5′ initiator caps, where cells are harvested 24 hours after exposure to mRNA.

FIG. 19 illustrates a schematic of MA004 and MA079.

FIG. 20 illustrates in vivo editing efficiency of ABE synthesized using 5′ initiator analogs in mouse.

FIG. 21a illustrates in vivo immunogenicity of ABE synthesized using 5′ initiator analogs as a measurement of TNF-α levels over time in mouse.

FIG. 21b illustrates in vivo immunogenicity of ABE synthesized using 5′ initiator analogs as a measurement of IFN-γ levels over time in mouse.

FIG. 21c illustrates in vivo immunogenicity of ABE synthesized using 5′ initiator analogs as a measurement of IFN-α levels over time in mouse.

FIG. 21d illustrates in vivo immunogenicity of ABE synthesized using 5′ initiator analogs as a measurement of IFN-β levels over time in mouse.

FIG. 21e illustrates in vivo immunogenicity of ABE synthesized using 5′ initiator analogs as a measurement of MIP-1B levels over time in mouse.

FIG. 21f illustrates in vivo immunogenicity of ABE synthesized using 5′ initiator analogs as a measurement of IP-10 levels over time in mouse.

FIG. 21g illustrates in vivo immunogenicity of ABE synthesized using 5′ initiator analogs as a measurement of IL-10 levels over time mouse.

FIG. 21h illustrates in vivo immunogenicity of ABE synthesized using 5′ initiator analogs as a measurement of IL-6 levels over time in mouse.

### DETAILED DESCRIPTION

Certain specific details of this description are set forth in order to provide a thorough understanding of various embodiments. However, one skilled in the art will understand that the present disclosure may be practiced without these details. In other instances, well-known structures and/or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure. The section headings used herein are for organizational purposes only and are not to be construed as limiting the subject matter described.

Messenger RNA (mRNA), encoding physiologically important proteins for therapeutic applications, has shown significant advantages over DNA-based plasmid and viral vectors for delivering genetic material. Such important advantages include: (i) potentially improved levels of safety when compared to the potential genome damage that can result from viral or plasmid integration, (ii) more immediate protein expression upon mRNA delivery (unlike delayed responses that generally occur with plasmids), (iii) robust dose-dependent control over expression of proteins, and (iv) is capable of facilitating simplification of large scale synthesis of mRNAs as compared to manufacturing of plasmid and viral vectors.

Messenger RNAs can be encoded for virtually any known protein and can be delivered to specific cells, tissues and organs by a variety of methods known to those skilled in the art. Once delivered, such mRNAs are capable of direct ribosomal protein expression within targeted cell or tissue resulting in the production of potentially many hundreds copies of the encoded proteins from a single mRNA molecule.

Several structural elements, present in active mRNA molecule, are utilized to translate the encoded proteins efficiently. One of these elements is the 5′-end region of mRNAs. In naturally occurring mRNA, the 5′ end-region comprises a Cap structure, which is an important feature of eukaryotic mRNA molecules (and some viruses). Such Cap structures are known to be involved in protein translation, 5′ exonuclease protection, splicing, and mRNA transport. A consistent structural element of a naturally occurring 5′ Cap is an inverted 7-methylguanosine (m⁷G) linked at the 5′ end of the mRNA through a triphosphate (ppp) bridge, and this phosphate bridge is linked to the first nucleotide (N₁) of the mRNA transcript. This 5′ Cap moiety generally represented by m⁷G(ppp)N₁ is called cap-0. Methylation of the 2′-hydroxyl on N₁ ribose ring (i.e., cap-1) is known to be capable of providing an identifier of self-RNA, which thereby serves to shield the mRNA from the innate immune system, which in turn is capable of improving protein expression. This m⁷G(ppp)N₁m cap-1 structure is a known conventional cap moiety used in in vitro transcription (IVT) of mRNAs.

Here we present novel mRNA 5′ end region motifs and initiators. These novel designs include modification of the purine base of the m⁷G moiety; phosphorothioate (PS) substitution in the triphosphate bridge and chemical modifications of phosphodiester linkages, substitution of the 5′ end region nucleotides with non-canonical bases, extension of the 5′ end region nucleotide oligomer, as well as chemical modification of the ribose rings. Specifically, these mRNA 5′ end region structures serve as the terminal 5′ end region of an mRNA and provide stability to the mRNA. The chemical structures of the motifs are capable of facilitating and/or modulating mRNA activity and rates of translation initiation and elongation; protecting mRNA by creating a barrier that prevents or interferes with mRNA decapping by 5′ exonuclease activity; impacting capping efficiency and reducing the formation of immune-stimulatory by-products, which can improve mRNA safety; and facilitating the mRNA manufacturability by modulating the binding affinity for DNA template during an IVT reaction.

### Definitions

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All references cited herein are incorporated by reference in their entirety as though fully set forth. Singleton et al., Dictionary of Microbiology and Molecular Biology 3rd ed., J. Wiley & Sons (New York, NY 2001); March, Advanced Organic Chemistry Reactions, Mechanisms and Structure 5th ed., J. Wiley & Sons (New York, NY 2001); and Sambrook and Russel, Molecular Cloning: A Laboratory Manual 3rd ed., Cold Spring Harbor Laboratory Press (Cold Spring Harbor, NY 2001), provide one skilled in the art with a general guide to many of the terms used in the present application.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

When indicating the number of substituents, the term “one or more” refers to the range from one substituent to the highest possible number of substitution, e.g. replacement of one hydrogen up to replacement of all hydrogens by substituents.

The term “optional” or “optionally” denotes that a subsequently described event or circumstance can but need not occur, and that the description includes instances where the event or circumstance occurs and instances in which it does not.

The term “nucleic acid” as used herein generally refers to one or more nucleobases, nucleosides, or nucleotides, and the term includes polynucleobases, polynucleosides, and polynucleotides. A nucleic acid can include polynucleotides, mononucleotides, and oligonucleotides. A nucleic acid can include DNA, RNA, or a mixture thereof, and can be single stranded, double stranded, or partially single or double stranded, and can form secondary structures. In some embodiments, a nucleic acid has multiple double-stranded segments and single stranded segments. For example, a nucleic acid may comprise a polynucleotide, e.g. a mRNA, with multiple double stranded segments within it.

The terms “mRNA sequence initiator”, “IVT mRNA sequence initiator” and “initiator” are used interchangeably herein to generally refer to a ribo- or deoxyribo- or chimeric ribo/deoxyribo-oligonucleotide, single stranded, may be naturally occurring or synthetic, and usually include a sequence of between about 2 to about 10 nucleotides, about 3 to about 8 nucleotides or about 3 to about 5 nucleotides. mRNA sequence initiators may contain one or more modification groups. mRNA sequence initiators may be primers, e.g. oligonucleotide primers. mRNA sequence initiators, for example, oligonucleotide primers, may include RNA, DNA, and/or other modified nucleosides. The skilled artisan is capable of designing and preparing mRNA sequence initiators that are appropriate for transcription of DNA template sequence.

mRNA sequence initiators may be capped primers or capped oligonucleotide analogs. For example, a capped mRNA sequence initiator may contain initiating capped oligonucleotide analogs or initiating capped oligonucleotides with Cap 0, Cap 1, Cap 2 or TMG-Cap structure on 5′-end. In some instances, a capped initiator, e.g., a capped primer or capped oligonucleotide analog has an unmodified or open 3′-OH group and it may be extended by RNA polymerase through the incorporation of an NTP onto the 3′-end. In some instances, an initiator as described herein can initiate in vitro transcription under the control of a promoter in a transcription system containing necessary components: DNA template (e.g. DNA plasmid), RNA polymerase, nucleoside 5′-triphosphates and appropriate buffer. An initiator may be a oligonucleotide carrying a terminal 3′-OH group that is a valid substrate for RNA polymerase. In certain embodiments, an initiator is a substrate for RNA polymerase and may be elongated by incorporation of NTP onto the 3′-end. In some embodiments, an initiator is complementary to the DNA template at the initiation site.

The term “unsubstituted” or “unmodified” in the context of mRNA sequence initiators and nucleoside triphosphates (NTPs) as used herein generally refers to an initiating capped initiator and NTPs that have not been modified.

The term “modified initiating capped initiator” as used herein generally refers to an initiating capped mRNA sequence initiator that contains one or more additional modification group(s) or moiety/moieties within the sequence initiator.

The term “modification group(s) or moiety/moeities” as used herein generally refers to any chemical moiety that may be attached or substituted to the mRNA sequence initiator, e.g., an initiating primer at locations, which include, but are not limited to, the sugar, nucleoside base, triphosphate bridge, and/or internucleotide phosphate (e.g., U.S. Patent Application No. 20070281308). The modification group of a capped initiator may be a group of any nature that is compatible with the process of transcription.

The term “internucleotide linkage” as used herein generally refers to the bond or bonds that connect two nucleosides of an initiator, e.g. an oligonucleotide primer or a nucleic acid and may be a natural phosphodiester linkage or a chemically modified nucleic acid backbone linkage.

The term “polynucleotide”, as used herein generally refers to a molecule comprising two or more linked nucleic acid subunits, e.g., nucleotides, and can be used interchangeably with “oligonucleotide”. For example, a polynucleotide may include one or more nucleotides selected from corresponding nucleosides carrying the nucleobase-adenine (A), cytosine (C), guanine (G), thymine (T) and uracil (U), or variants and combinations thereof. A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides include nucleotides in which the sugar is ribose. Deoxyribonucleotides include nucleotides in which the sugar is deoxyribose. A nucleotide can be a nucleoside monophosphate, nucleoside diphosphate, nucleoside triphosphate or a nucleoside polyphosphate. For example, a nucleotide can be a deoxyribonucleoside polyphosphate, such as a deoxyribonucleoside triphosphate (dNTP), Exemplary dNTPs include deoxyadenosine triphosphate (dATP), deoxycytidine triphosphate (dCTP), deoxyguanosine triphosphate (dGTP), uridine triphosphate (dUTP) and deoxythymidine triphosphate (dTTP). dNTPs can also include detectable tags, such as luminescent tags or markers (e.g., fluorophores). For example, a nucleotide can be a purine (e.g., A or G, or variant thereof) or a pyrimidine (e.g., C, T or U, or variant thereof). In some examples, a polynucleotide is deoxyribonucleic acid (DNA), ribonucleic acid (RNA), or derivatives or variants thereof. Exemplary polynucleotides include, but are not limited to, short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), and heteronuclear RNA (hnRNA), and encompasses both the nucleotide sequence and any structural embodiments thereof, such as single-stranded, double-stranded, triple-stranded, helical, hairpin, stem loop, bulge, etc. In some cases, a polynucleotide is circular. A polynucleotide can have various lengths. For example, a polynucleotide can have a length of at least about 7 bases, 8 bases, 9 bases, 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. For example, polynucleotide sequences may comprise isolated and purified DNA/RNA molecules, synthetic DNA/RNA molecules, and/or synthetic DNA/RNA analogs.

Polynucleotides can include one or more nucleotide variants, including nonstandard nucleotide(s), non-natural nucleotide(s), nucleotide analog(s) and/or modified nucleotides including acyclic and carbocyclic nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012, 8(7):612-4, which is herein incorporated by reference for all purposes.

As used herein, the terms “polypeptide”, “protein” and “peptide” are used interchangeably and refer to a polymer of amino acid residues linked via peptide bonds and which may be composed of two or more polypeptide chains. The terms “polypeptide”, “protein” and “peptide” refer to a polymer of at least two amino acid monomers joined together through amide bonds. An amino acid may be the L-optical isomer or the D-optical isomer. More specifically, the terms “polypeptide”, “protein” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant.

As used herein, “hybridize” refers to a process where initiating a capped mRNA sequence initiator anneals to a DNA template in accordance with Watson-Crick base pairing rules under appropriately stringent conditions during a transcription reaction. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to determine the appropriate stringency of hybridization/washing conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J, all of which are incorporated herein by reference in their entireties. In certain embodiments, hybridizations may occur between nucleic acid molecules of 20-100 nucleotides in length. In some embodiments, hybridization may occur between at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 consecutive nucleotides. In some embodiments, the hybridizing nucleic acid molecules may contain up to 1, 2, 3, 4, 5, 6, 7, 8, 9, 10, 11, 12, 13, 14, 15, 16, 17, 18, 19, or 20 mismatches that are tolerated.

As used herein, “complement,” “complementary,” or “complementarity” in the context of a complex of, for example, an initiating capped oligonucleotide primer and a DNA template refers to standard Watson/Crick base pairing rules. For example, the sequence “5′-A-G-T-C-3′” is complementary to the sequence “3′-T-C-A-G-5′.” Certain non-natural or synthetic nucleotides may be included in the nucleic acids described herein; these include but not limited to, base and sugar modified nucleosides, nucleotides, and nucleic acids, such as inosine, 7-deazaguanosine, 2′-O-methylguanosine, 2′-fluoro-2′-deoxycytidine, pseudouridine, Locked Nucleic Acids (LNA), and Peptide Nucleic Acids (PNA). Complementarity does not need to be perfect; duplexes may contain mismatched base pairs, degenerative, or unmatched nucleotides. Those skilled in the art can determine duplex stability empirically considering a number of variables including, for example, the length of the oligonucleotide, base composition and sequence of the oligonucleotide, incidence of mismatched base pairs, ionic strength, components of the hybridization buffer and reaction conditions.

Complementarity may be “complete” or “total” where all of the nucleotide bases of two nucleic acid strands are matched according to recognized base pairing rules, it may be “partial” in which only some of the nucleotide bases of an capped mRNA sequence initiator and a DNA target are matched according to recognized base pairing rules or it may be “absent” where none of the nucleotide bases of two nucleic acid strands are matched according to recognized base pairing rules. The degree of complementarity between a capped mRNA sequence initiator, e.g. a capped primer, and a DNA template may have a significant effect on the strength of hybridization between the initiating capped oligonucleotide and the DNA template and correspondingly the efficiency of the reaction. The term complementarity may also be used in reference to individual nucleotides. For example, a particular nucleotide within an oligonucleotide may be noted for its complementarity, or lack thereof, to a nucleotide within another strand, in contrast or comparison to the complementarity between the rest of an capped mRNA sequence initiator and a DNA strand.

As used herein the term “complete”, “total” or “perfectly” complementary means that each of the nucleotide bases of a capped mRNA sequence initiator and a DNA target are matched exactly according to recognized base pairing rules.

As used herein, the term “substantially complementary” refers to two sequences that hybridize under stringent hybridization conditions. Those skilled in the art will understand that substantially complementary sequences need not hybridize along their entire length. In particular, substantially complementary sequences may comprise a contiguous sequence of bases that do not hybridize to a target sequence and may be positioned 3′ or 5′ to a contiguous sequence of bases that hybridize under stringent hybridization conditions to the target sequence.

As used herein, the term “nucleoside” includes all naturally occurring nucleosides, including all forms of nucleoside bases and furanosides found in nature. Base rings most commonly found in naturally occurring nucleosides are purine and pyrimidine rings. Naturally occurring purine rings include, for example, adenine, guanine, and N⁶-methyladenine. Naturally occurring pyrimidine rings include, for example, cytosine, thymine, 5-methylcytosine, pseudouracyl. Naturally occurring nucleosides for example include, but are not limited to, ribo, 2′-O-methyl or 2′-deoxyribo derivatives of adenosine, guanosine, cytidine, thymidine, uridine, inosine, 7-methylguanosine or pseudouridine.

As used herein, the terms “nucleoside analogs,” “modified nucleosides,” or “nucleoside derivatives” include synthetic nucleosides as described herein. Nucleoside derivatives also include nucleosides having modified base or/and sugar moieties, with or without protecting groups and include, for example, 2′-deoxy-2′-fluorouridine, 5-fluorouridine and the like. The compounds and methods provided herein include such base rings and synthetic analogs thereof, as well as unnatural heterocycle-substituted base sugars, and acyclic substituted base sugars. Other nucleoside derivatives that may be utilized with the present disclosure include, for example, LNA nucleosides, halogen-substituted purines (e.g., 6-fluoropurine), halogen-substituted pyrimidines, N⁶-ethyladenine, N⁴-(alkyl)-cytosines, 5-ethylcytosine, and the like (U.S. Pat. No. 6,762,298).

As used herein, the terms “universal base,” “degenerate base,” “universal base analog” and “degenerate base analog” include, for example, a nucleoside analog with an artificial base which is, in certain embodiments, recognizable by RNA polymerase as a substitute for one of the natural NTPs (e.g., ATP, UTP, CTP and GTP) or other specific NTP. Universal bases or degenerate bases are disclosed in Loakes, D., Nucleic Acids Res., 29:2437-2447 (2001); Crey-Desbiolles, C., et. al., Nucleic Acids Res., 33:1532-1543 (2005); Kincaid, K., et. al., Nucleic Acids Res., 33:2620-2628 (2005); Preparata, F P, Oliver, J S, J Comput. Biol. 753-765 (2004); and Hill, F., et. al., Proc Natl Acad. Sci. USA, 95:4258-4263 (1998)).

As used herein, the term “modified NTP” refers to a nucleoside 5′-triphosphate having a chemical moiety group bound at any position, including the sugar, base, triphosphate chain, or any combination of these three locations. Examples of such NTPs can be found, for example in “Nucleoside Triphosphates and Their Analogs: Chemistry, Biotechnology and Biological Applications,” Vaghefi, M., ed., Taylor and Francis, Boca Raton (2005).

As used herein, the term “specific” when used in reference to a 5′ capped mRNA sequence initiator sequence and its ability to hybridize to a DNA template is a sequence that has at least 50% sequence identity with a portion of the DNA template when the capped mRNA sequence initiator and DNA strand are aligned. Higher levels of sequence identity that may be preferred include at least 75%, at least 80%, at least 85%, at least 90%, at least 95%, at least 99%, and most preferable 100% sequence identity.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide oligonucleotides having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N6-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N6-(isopentyl)adenine, N6-(methyl)adenine, N6,N6-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N4-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-methylpseudouracil (N1-methylpseudouracil), 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, l-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted-1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ori/zo-substituted-6-phenyl-pyrrolo-pyrimidin-2-on-3-yl, bis-ori/zo-substituted-6-phenyl-pyrrolo-pyrimidin-2-on-3-yl, para-(aminoalkylhydroxy)-6-phenyl-pyrrolo-pyrimidin-2-on-3-yl, ori/zo-(aminoalkylhydroxy)-6-phenyl-pyrrolo-pyrimidin-2-on-3-yl, bis-ori/zo-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed. A universal nucleobase is any nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447, incorporated herein by reference in its entirety). Further nucleobases include those disclosed in U.S. Pat. No. 3,687,808; those disclosed in International Application No. PCT U.S. Ser. No. 09/038,425, filed Mar. 26, 2009; those disclosed in the Concise Encyclopedia Of Polymer Science And Engineering, pages 858-859, Kroschwitz, J. I, ed. John Wiley & Sons, 1990; those disclosed by English et al, Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

As used herein, the term “biological sample” means any biological material from which polynucleotides, polypeptides, biomarkers, and/or metabolites can be prepared or can be extracted out and examined. Non-limiting examples encompasses whole blood, plasma, saliva, cheek swab, fecal specimen, urine specimen, cell mass, or any other bodily fluid or tissue.

The terms “administer,” “administering”, “administration,” and the like, as used herein, refer to the methods that may be used to enable delivery of compounds or compositions to the desired site of biological action. These methods include, but are not limited to oral routes (p.o.), intraduodenal routes (i.d.), parenteral injection (including intravenous (i.v.), subcutaneous (s.c.), intraperitoneal (i.p.), intramuscular (i.m.), intravascular or infusion (inf.)), topical (top.) and rectal (p.r.) administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated; for example a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases.

The terms “enhance” or “enhancing,” as used herein, means to increase or prolong either in amount, potency or duration a desired effect.

As used herein, “carbohydrate” refers to a compound which is either a carbohydrate per se made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C5 and above (preferably C5-C8) sugars; di- and trisaccharides include sugars having two or three monosaccharide units (preferably C5-C8).

The term “monosaccharide” embraces radicals of allose, altrose, arabinose, cladinose, erythrose, erythrulose, fructose, D-fucitol, L-fucitol, fucosamine, fucose, fuculose, galactosamine, D-galactosaminitol, N-acetyl-galctosamine, galactose, glucosamine, N-acetyl-glucosamine, glucosaminitol, glucose, glucose-6-phosphate gulose glyceraldehyde, L-glycero-D-mannos-heprose, glycerol, glycerone, gulose idose, lyxose, mannosamine, mannose, mannose-6-phosphate, psicose, quinovose, quinovosamine, rhamnitol, rhamnosamine, rhamnose, ribose, ribulose, sedoheptulose, sorbose, tagatose, talose, tararic acid, throse, xylose and xylulose. The monosaccharide can be in D- or L-configuration. The monosaccharide may further be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), a imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, an aldaric acid, and so forth. Amino sugars include amino monosaccharides, preferably galactosamine, glusamine, mannosamine, fucosmine, quinavosamine, neuraminic acid, muramic acid, lactosediamine, acosamine, bacillosamine, daunosamine, desosamine, forosamine, garosamine, kanosamine, kanosamine, mycaminose, myosamine, persosamine, pneumosamine, purpurosamine, rhodosmine. It is understood that the monosaccharide and the like can be further substituted.

The terms “disaccharide”, “trisaccharide” and “polysaecharide” embrace radicals of abequose, acrabose, amicetose, amylopectin, amylose, apiose, arcanose, ascarylose, ascorbic acid, boivinose, cellobiose, cellotriose, cellulose, chacotriose, chalcose, chitin, colitose, cyclodextrin, cymarose, dextrin, 2-deoxyribose, 2-deoxyglucose diginose, digitalose, digitoxose, evalose, evemitrose, fructooligosachharide, galto-oligosaccharide, gentianose, genitiobiose, glucan, gluicogen, glylcogen, hamamelose, heparin, inulin, isolevoglucosenone, isomaltose, isomaltotriose, isopanose, kojibiose, lactose, lactosamine, lactosediamine, laminarabiose, levoglucosan, levoglucosenone, β-maltose, maltriose, mannan-oligosacchardie, amnninotriose, melezitose, melibiose, muramic acid, mycarose, mycinose, neuaminic acid, migerose, nojirimycon, noviose, oleandrose, panose, paratose, planteose, primeverose, raffinose, rhodone, rutinose, oleandrose, panose, paratose, planteose, primeverose, raffinose, rhodinose, rutinose, sarmentose, sedoheptulose, sedoheptulosan, solatriose, sophorose, stachyose, streptose, sucrose, α,α-trehalose, trahalosamine, turanose, tyvelose, xylobiose, umbelliferose and the like. Further, it is understood that the “disaccharide”, “trisaccharide” and “polysaccharide” and the like can further substituted. Disaccharide also includes amino sugars and their derivatives, particularly, a mycaminose derivatized a the C-4′ position or a 4 deoxy-3-amino-glucose derivatized at the C-6′ position.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a non-human animal is a mouse.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically. The term “treating” further encompasses the concept of “prevent,” “preventing,” and “prevention,” as stated below. It is appreciated that, although not precluded, treating a disorder or condition does not require that the disorder, condition, or symptoms associated therewith be completely eliminated.

The term “preventing” or “prevention” of a disease state denotes causing the clinical symptoms of the disease state not to develop in a subject that can be exposed to or predisposed to the disease state, but does not yet experience or display symptoms of the disease state.

The terms “pharmaceutical composition” and “pharmaceutical formulation” (or “formulation”) are used interchangeably and denote a mixture or a solution comprising a therapeutically effective amount of an active pharmaceutical ingredient together with one or more pharmaceutically acceptable excipients to be administered to a subject, e.g., a human in need thereof.

The term “pharmaceutical combination” as used herein, means a product that results from mixing or combining more than one pharmaceutically active ingredient and includes both fixed and non-fixed combinations of the active ingredients. The term “fixed combination” means that the active ingredients, e.g., a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., administration of three or more active ingredients.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

The terms “pharmaceutically acceptable excipient”, “pharmaceutically acceptable carrier”, “pharmaceutically acceptable vehicle” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products.

The term “base editing” and “base correction” are used interchangeably to indicate a base change or mutation at a target sequence within the target gene leading to base modification. In certain embodiments, base editing occurs at a single base of the target sequence. In preferred embodiments, base editing does not involve double strand breaks of the target sequence.

As used herein, the term “siRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. As used herein, the term siRNA includes microRNAs and pre-microRNAs. As used herein, the terms “siRNA activity” and “RNAi activity” refer to gene silencing by an siRNA.

The term “2′-O-methoxyethyl” (also 2′-MOE, 2′-O(CH₂)₂—OCH₃ and 2′-O-(2-methoxyethyl)) refers to an O-methoxy-ethyl modification of the 2′ position of a furosyl ring. A 2′-O-methoxyethyl modified sugar is a modified sugar.

The term “2′-O-methoxyethyl nucleotide” means a nucleotide comprising a 2′-O-methoxyethyl modified sugar moiety.

The term “5-methylcytosine” means a cytosine modified with a methyl group attached to the 5′ position. A 5-methylcytosine is a modified nucleobase.

The term “oxo” refers to the ═O substituent.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “alkylamino” refers to a radical of the formula —NHR or —NRR where each R is, independently, an alkyl radical as defined above. Unless stated otherwise specifically in the specification, an alkylamino group may be optionally substituted as described below.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂. Depending on the structure, an alkenyl group can be monovalent or divalent (i.e., an alkenylene group).

The term “alkynyl” refers to a type of alkyl group in which at least one carbon-carbon triple bond is present. Accordingly, “alkynylene” can refer to a divalent alkynyl group. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be monovalent or divalent (i.e., an “arylene” group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein. In some embodiments, an aryl group is a C₆-C₁₄ aryl. In some embodiments, an aryl group is a C₆-C₁₀ aryl.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

The term “haloalkyl” denotes an alkyl group wherein at least one of the hydrogen atoms of the alkyl group has been replaced by same or different halogen atoms, particularly fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl. The term “perhaloalkyl” denotes an alkyl group where all hydrogen atoms of the alkyl group have been replaced by the same or different halogen atoms.

The term “heteroalkylene” refers to an alkyl radical as described above where one or more carbon atoms of the alkyl is replaced with a 0, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted. As used herein, the term “heterocycloalkylene” can refer to a divalent heterocycloalkyl group.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein. Depending on the structure, a heteroaryl group can be monovalent or divalent (i.e., a “heteroarylene” group).

The term “substituted,” “substituent” or the like, unless otherwise indicated, can refer to the replacement of one or more hydrogen radicals in a given structure individually and independently with the radical of a specified substituent including, but not limited to: D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, —SC₁-C₄ alkyl, —S(═O)C₁-C₄ alkyl, and —S(═O)₂(C₁-C₄ alkyl). In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

“About” means within +10% of a value. For example, if it is stated, “a marker may be increased by about 50%”, it is implied that the marker may be increased between 45%-55%.

“Active pharmaceutical agent” means the substance or substances in a pharmaceutical composition that provide a therapeutic benefit when administered to an individual.

“Dosage unit” means a form in which a pharmaceutical agent is provided, e.g. pill, tablet, or other dosage unit known in the art. In certain embodiments, a dosage unit is a vial containing lyophilized antisense oligonucleotide. In certain embodiments, a dosage unit is a vial containing reconstituted antisense oligonucleotide.

“Dose” means a specified quantity of a pharmaceutical agent provided in a single administration, or in a specified time period. In certain embodiments, a dose can be administered in one, two, or more boluses, tablets, or injections. For example, in certain embodiments where subcutaneous administration is desired, the desired dose requires a volume not easily accommodated by a single injection, therefore, two or more injections can be used to achieve the desired dose. In certain embodiments, the pharmaceutical agent is administered by infusion over an extended period of time or continuously. Doses can be stated as the amount of pharmaceutical agent per hour, day, week, or month. Doses can also be stated as the mass of pharmaceutical drug product or drug substance per mass of subject tissue (e.g., mg/kg or g/kg).

“Modified internucleoside linkage” refers to a substitution or any change from a naturally occurring internucleoside bond. For example, a phosphorothioate linkage is a modified internucleoside linkage.

“Modified nucleobase” refers to any nucleobase other than adenine, cytosine, guanine, thymidine, or uracil. For example, 5-methylcytosine is a modified nucleobase. An “unmodified nucleobase” means the purine bases adenine (A) and guanine (G), and the pyrimidine bases thymine (T), cytosine (C), and uracil (U).

“Modified nucleoside” means a nucleoside having at least one modified sugar moiety, and/or modified nucleobase.

“Modified nucleotide” means a nucleotide having at least one modified sugar moiety, modified internucleoside linkage and/or modified nucleobase.

“Modified oligonucleotide” means an oligonucleotide comprising at least one modified nucleotide.

“Modified sugar” refers to a substitution or change from a natural sugar. For example, a 2′-O-methoxyethyl modified sugar is a modified sugar.

“Motif” means the pattern of chemically distinct regions in an antisense compound.

“Statin” means an agent that inhibits the activity of HMG-CoA reductase.

“Symptom of cardiovascular disease or disorder” means a phenomenon that arises from and accompanies the cardiovascular disease or disorder and serves as an indication of it. For example, angina; chest pain; shortness of breath; palpitations; weakness; dizziness; nausea; sweating; tachycardia; bradycardia; arrhythmia; atrial fibrillation; swelling in the lower extremities; cyanosis; fatigue; fainting; numbness of the face; numbness of the limbs; claudication or cramping of muscles; bloating of the abdomen; or fever are symptoms of cardiovascular disease or disorder.

“Target nucleic acid,” and “target sequence” refer to a nucleic acid capable of being targeted by a genome editing composition. For example, a target DNA sequence within or adjacent to the ANGPTL3 gene may be targeted by a guide nucleotide associated with a Cas9 nuclease.

Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Methods for detection and/or measurement of RNA in biological material are well known in the art and include, but are not limited to, Northern-blotting, RNA protection assay, RT PCR. Suitable methods are described in Molecular Cloning: A Laboratory Manual (Fourth Edition) By Michael R. Green, Joseph Sambrook, Peter MacCallum 2012, 2,028 pp, ISBN 978-1-936113-42-2.

A ribonucleoprotein (RNP) refers to a nucleoprotein that contains RNA. A RNP can be a complex of a ribonucleic acid and an RNA-binding protein. Such a combination can also be referred to as a protein-RNA complex. These complexes can function in a number of biological functions that include, but are not limited to, DNA replication, DNA modification, gene expression, metabolism and modification of RNA, and pre-mRNA splicing.

As used herein, the term “biomarker” or “marker” are used interchangeably to refer to any biochemical marker, serological marker, genetic marker, or other clinical or echographic characteristic that can be used to classify a sample from a patient as being associated with an pathological condition, such as a cardiovascular disease or disorder.

As used herein, the term “antibody” includes but is not limited to a population of immunoglobulin molecules, which can be polyclonal or monoclonal and of any class and isotype, or a fragment of an immunoglobulin molecule. There are five major classes of immunoglobulins: IgA, IgD, IgE, IgG, and IgM, and several of these may be further divided into subclasses (isotypes), e.g., IgG1, IgG2, IgG3, IgG4, IgA1 (human), IgA2 (human), IgAa (canine), IgAb (canine), IgAc (canine), and IgAd (canine). Such fragment generally comprises the portion of the antibody molecule that specifically binds an antigen. For example, a fragment of an immunoglobulin molecule known in the art as Fab, Fab′ or F(ab′)₂ is included within the meaning of the term antibody.

The term “label,” as used herein, refers to a detectable compound, composition, or solid support, which can be conjugated directly or indirectly (e.g., via covalent or non-covalent means, alone or encapsulated) to a monoclonal antibody or a protein. The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current disclosure could be, but is not limited to alkaline phosphatase; glucose-6-phosphate dehydrogenase (“G6PDH”); horseradish peroxidase (HRP); chemiluminescers such as isoluminol, fluorescers such as fluorescein and rhodamine compounds; ribozymes; and dyes. The label may also be a specific binding molecule which itself may be detectable (e.g., biotin, avidin, streptavidin, digioxigenin, maltose, oligohistidine, e.g., hex-histidine, 2,4-dinitrobenzene, phenylarsenate, ssDNA, dsDNA, and the like). The utilization of a label produces a signal that may be detected by means such as detection of electromagnetic radiation or direct visualization, and that can optionally be measured.

“Substantial binding” or “substantially binding” refer to an amount of specific binding or recognizing between molecules in an assay mixture under particular assay conditions. In its broadest aspect, substantial binding relates to the difference between a first molecule's incapability of binding or recognizing a second molecule, and the first molecules capability of binding or recognizing a third molecule, such that the difference is sufficient to allow a meaningful assay to be conducted to distinguish specific binding under a particular set of assay conditions, which includes the relative concentrations of the molecules, and the time and temperature of an incubation. In another aspect, one molecule is substantially incapable of binding or recognizing another molecule in a cross-reactivity sense where the first molecule exhibits a reactivity for a second molecule that is less than 25%, e.g. less than 10%, e.g., less than 5% of the reactivity exhibited toward a third molecule under a particular set of assay conditions, which includes the relative concentration and incubation of the molecules. Specific binding can be tested using a number of widely known methods, e.g, an immunohistochemical assay, an enzyme-linked immunosorbent assay (ELISA), a radioimmunoassay (RIA), or a western blot assay.

As used herein, the term “substantially the same amino acid sequence” includes an amino acid sequence that is similar, but not identical to, the naturally-occurring amino acid sequence. For example, an amino acid sequence, e.g., polypeptide, that has substantially the same amino acid sequence as a flagellin protein can have one or more modifications such as amino acid additions, deletions, or substitutions relative to the amino acid sequence of the naturally-occurring flagellin protein, provided that the modified polypeptide retains substantially at least one biological activity of flagellin such as immunoreactivity. The “percentage similarity” between two sequences is a function of the number of positions that contain matching residues or conservative residues shared by the two sequences divided by the number of compared positions times 100. In this regard, conservative residues in a sequence is a residue that is physically or functionally similar to the corresponding reference residue, e.g., that has a similar size, shape, electric charge, chemical properties, including the ability to form covalent or hydrogen bonds, or the like.

The term “targeting moiety” refers to any molecule that provides an enhanced affinity for a selected target, e.g., a cell, cell type, tissue, organ, region of the body, or a compartment, e.g., a cellular, tissue or organ compartment. Some exemplary targeting moieties include, but are not limited to, antibodies, antigens, carbohydrate base moieties, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

The term “heterologous” refers to any two or more nucleic acid or polypeptide sequences that are not normally found in the same relationship to each other in nature. For instance, a heterologous nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

As used herein, the term “fragment” includes a peptide, polypeptide or protein segment of amino acids of the full-length protein, provided that the fragment retains reactivity with at least one antibody in sera of disease patients.

An “epitope” is the antigenic determinant on a polypeptide that is recognized for binding by a paratope on antibodies specific to the polypeptide, for example, an IBD-associated antibody.

The term “clinical factor” includes a symptom in a patient that is associated with a disease. Examples of clinical factors for cardiovascular disease include, without limitation, angina; chest pain; shortness of breath; palpitations; weakness; dizziness; nausea; sweating; tachycardia; bradycardia; arrhythmia; atrial fibrillation; swelling in the lower extremities; cyanosis; fatigue; fainting; numbness of the face; numbness of the limbs; claudication or cramping of muscles; bloating of the abdomen; or fever. In some embodiments, a diagnosis of a cardiovascular disease is based upon a combination of analyzing the presence or level of one or more markers in a patient using statistical algorithms and determining whether the patient has one or more clinical factors.

The term “prognosis” includes a prediction of the probable course and outcome of a pathological condition, for example a cardiovascular disease, or the likelihood of recovery from the disease. In some embodiments, the use of statistical algorithms provides a prognosis of cardiovascular disease in a patient. For example, the prognosis can be surgery, development of one or more clinical factors, development of intestinal cancer, or recovery from the disease.

The term “CRISPR-Cas system” referred herein includes a CRISPR-associated protein translated from an mRNA encoding the said protein and a single guide RNA. In some embodiments, the CRISPR-associated protein may have inherent endonucleolytic activity. In some embodiments, the CRISPR-Cas system facilitate guide RNA mediated gene alteration. protein or proteins produced from protein-encoded mRNA can facilitate base or nucleobase and/or gene editing within a targeted segment of a gene of interest.

Provided herein are methods and compositions for targeted delivery of therapeutic agents such as nucleic acid agents. The therapeutic agents as used herein may be connected to or associated with a targeting moiety to assist targeted delivery. For example, the therapeutic agent and the targeting moiety may form a conjugate. The therapeutic agent may comprise a nucleic acid guided programmable nuclease system complexed with nucleic acids, such as guide RNAs. In some embodiments, the guide RNAs may be chemically modified. In some embodiments, the modified guide RNAs can be used for the preparation of a medicament for the treatment of any disease, disorder or condition relating to a gene where the gene may be altered, manipulated, edited, and modified by insertion or deletion of DNA. According to a further aspect of the disclosure, the modified guide RNA may be used for altering genes by deleting, substituting, repairing or inserting on or more nucleotide or a segment of DNA. This can be done in microorganisms, or animals, in particular mammals and more particularly in humans. Human cells or tissue may be genetically altered or amended using the guide RNAs of the present disclosure and the CRISPR/Cas system known in the art in vitro and then inserted back into the patient in need thereof. In another aspect of the disclosure there is provided a pharmaceutical composition comprising a modified guide RNA according to the disclosure and a CRISPR-Cas system and a pharmaceutically acceptable carrier or excipient. The pharmaceutical composition may include a vector or a cell with the modified guide RNA of the disclosure.

### Compounds

In one aspect, described herein is an IVT mRNA sequence initiator of Formula (I) or a salt or solvate thereof:

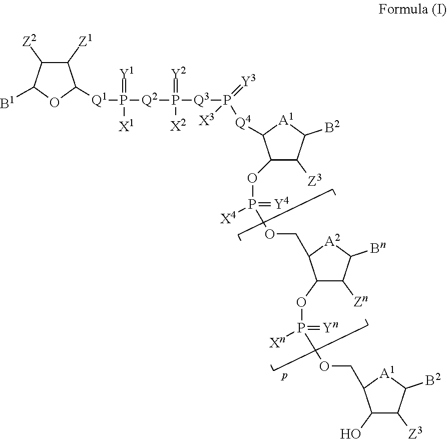

wherein
B¹ is

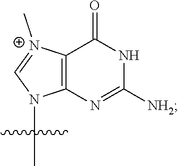 [SMILES: *n1c[n+](C)c2c(=O)[nH]c(N)nc21]

each B², B³, and Bⁿ is independently a natural, a modified, or an unnatural nucleobase;
each Z¹ and Z² is independently hydrogen, fluorine, —OH, —SH, —CH₃, —CH₂CH₃, —OCH₃, —OCH₂CH₃, —SCH₃, —NH₂, NHCH₃, or NHC(═O)CH₃;
each Z³, Z⁴, and Zⁿ is independently hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6,
provided that (i) Z¹, Z², and Z³ are hydrogen; (ii) Q¹ and Q⁴ are —CH₂O—; (iii) Q² and Q³ are oxygen; (iv) at least one of X¹, X², X³, X⁴, and Xⁿ is —SH or —S—; (v) at least one of Y, Y², Y³, Y⁴, and Yⁿ is ═S; or (vi) at least one of A, A¹, and A² is —S—.

In some embodiments, the IVT mRNA sequence initiator has a structure of Formula (I-a):

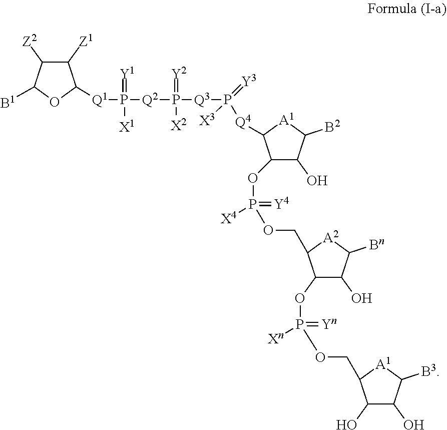

In some embodiments, the IVT mRNA sequence initiator has a structure of Formula (I-b):

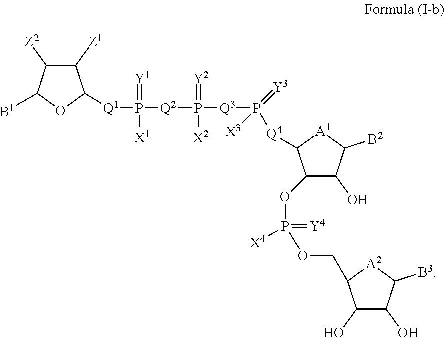

In some embodiments, the IVT mRNA sequence initiator has a structure of Formula (I-c):

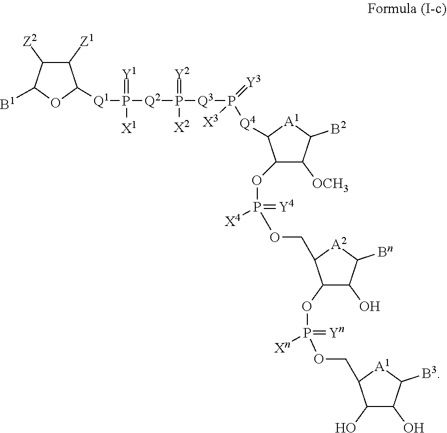

In some embodiments, the IVT mRNA sequence initiator has a structure of Formula (I-d):

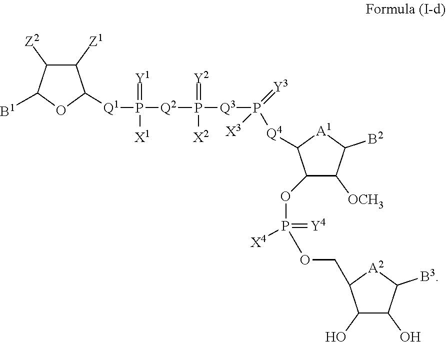

In some embodiments, the IVT mRNA sequence initiator of Formula (I-d) has a structure of

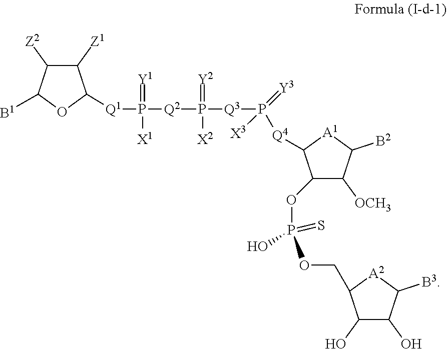

In some embodiments, the IVT mRNA sequence initiator of Formula (I-d) has a structure of

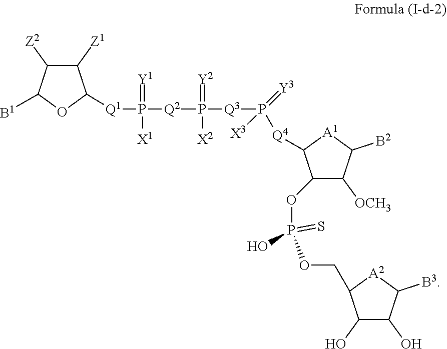

In some embodiments, the IVT mRNA sequence initiator has a structure of Formula (I-e):

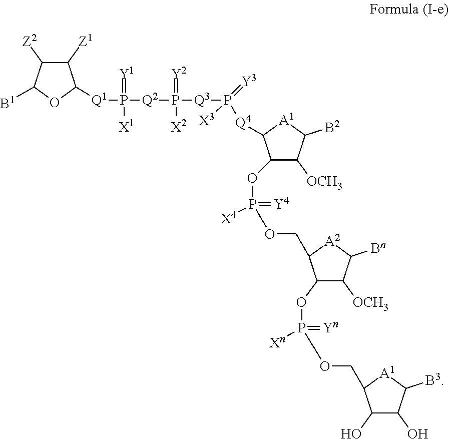

In some embodiments, the IVT mRNA sequence initiator has a structure of Formula (I-f):

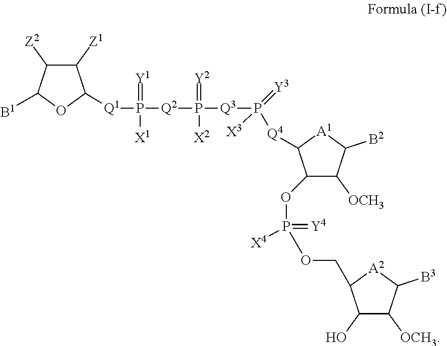

In some embodiments, the IVT mRNA sequence initiator has a structure of Formula (I-g):

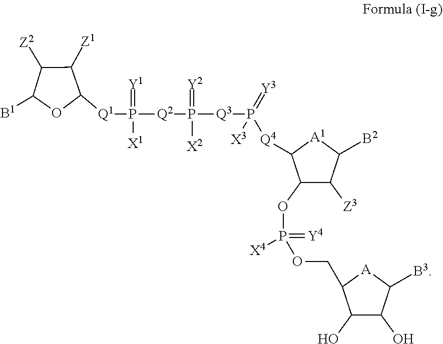

In some embodiments, the IVT mRNA sequence initiator has a structure of Formula (I-h):

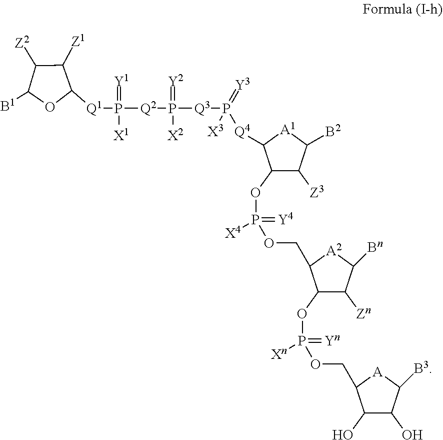

In some embodiments of a compound of Formula (I), (I-a), (I-b), (I-c), (I-d), (I-e), (I-f), (I-g) or (I-h) is a salt. In some embodiments, the salt is an alkali metal salt. In some embodiments, the salt is a sodium salt. In some embodiments, the salt is an ammonium salt.

In some embodiments of a compound of Formula (I), (I-a), (I-b), (I-c), (I-d), (I-e), (I-f), (I-g) or (I-h), Z¹ is hydrogen, fluorine, —OH, —SH, —CH₃, —CH₂CH₃, —OCH₃, —OCH₂CH₃, —SCH₃, —NH₂, NHCH₃, or NHC(═O)CH₃. In some embodiments, Z¹ is hydrogen. In some embodiments, Z¹ is F. In some embodiments, Z¹ is —OH. In some embodiments, Z¹ is —SH. In some embodiments, Z¹ is —CH₃. In some embodiments, Z¹ is —CH₂CH₃. In some embodiments, Z¹ is —OCH₃. In some embodiments, Z¹ is —OCH₂CH₃. In some embodiments, Z¹ is —SCH₃. In some embodiments, Z¹ is —NH₂. In some embodiments, Z¹ is NHCH₃. In some embodiments, Z¹ is NHC(═O)CH₃.

In some embodiments of a compound of Formula (I), (I-a), (I-b), (I-a), (I-d), (I-e), (I-f), (I-g) or (I-h), Z² is hydrogen, fluorine, —OH, —SH, —CH₃, —CH₂CH₃, —OCH₃, —OCH₂CH₃, —SCH₃, —NH₂, NHCH₃, or NHC(═O)CH₃. In some embodiments, Z² is hydrogen. In some embodiments, Z² is F. In some embodiments, Z² is —OH. In some embodiments, Z² is —SH. In some embodiments, Z² is —CH₃. In some embodiments, Z² is —CH₂CH₃. In some embodiments, Z² is —OCH₃. In some embodiments, Z² is —OCH₂CH₃. In some embodiments, Z² is —SCH₃. In some embodiments, Z² is —NH₂. In some embodiments, Z² is NHCH₃. In some embodiments, Z² is NHC(═O)CH₃.

In some embodiments of a compound of Formula (I), (I-a), (I-b), (I-c), (I-d), (I-e), (I-f), (I-g) or (I-h), Z³ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z³ is hydrogen. In some embodiments, Z³ is fluorine. In some embodiments, Z³ is —OH. In some embodiments, Z³ is —SH. In some embodiments, Z³ is —CH₃. In some embodiments, Z³ is —CH₂CH₃. In some embodiments, Z³ is —OCH₂OCH₃. In some embodiments, Z³ is —OCH₂CH₂CH₃. In some embodiments, Z³ is —OCH(CH₃)₂. In some embodiments, Z³ is —SCH₃. In some embodiments, Z³ is —OCH₂CH₂OCH₃.

In some embodiments of a compound of Formula (I), (I-a), (I-b), (I-c), (I-d), (I-e), (I-f), (I-g) or (I-h), Z⁴ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z⁴ is hydrogen. In some embodiments, Z⁴ is fluorine. In some embodiments, Z⁴ is —OH. In some embodiments, Z⁴ is —SH. In some embodiments, Z⁴ is —CH₃. In some embodiments, Z⁴ is —CH₂CH₃. In some embodiments, Z⁴ is —OCH₂OCH₃. In some embodiments, Z⁴ is —OCH₂CH₂CH₃. In some embodiments, Z⁴ is —OCH(CH₃)₂. In some embodiments, Z⁴ is —SCH₃. In some embodiments, Z⁴ is —OCH₂CH₂OCH₃.

In some embodiments of a compound of Formula (I), (I-a), (I-b), (I-c), (I-d), (I-e), (I-f), (I-g) or (I-h), Zⁿ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Zⁿ is hydrogen. In some embodiments, Zⁿ is fluorine. In some embodiments, Zⁿ is —OH. In some embodiments, Zⁿ is —SH. In some embodiments, Zⁿ is —CH₃. In some embodiments, Zⁿ is —CH₂CH₃. In some embodiments, Zⁿ is —OCH₂OCH₃. In some embodiments, Zⁿ is —OCH₂CH₂CH₃. In some embodiments, Zⁿ is —OCH(CH₃)₂. In some embodiments, Zⁿ is —SCH₃. In some embodiments, Zⁿ is —OCH₂CH₂OCH₃.

In some embodiments of a compound of Formula (I), (I-a), (I-b), (I-c), (I-d), (I-e), (I-f), (I-g) or (I-h), B² is independently a natural, a modified, or an unnatural nucleobase. In some embodiments B² is guanine. In some embodiments B² is adenine. In some embodiments, B² is cytosine. In some embodiments, B² is uracil, In some embodiments, B² is thymine, In some embodiments, B² is hypoxanthine. In some embodiments, B² is purine.

In some embodiments of a compound of Formula (I), (I-a), (I-b), (I-c), (I-d), (I-e), (I-f), (I-g) or (I-h), B³ is independently a natural, a modified, or an unnatural nucleobase. In some embodiments, B³ is guanine. In some embodiments, B³ is adenine. In some embodiments, B³ is cytosine. In some embodiments, B³ is uracil, In some embodiments, B³ is thymine, In some embodiments, B³ is hypoxanthine. In some embodiments, B³ is purine.

In some embodiments of a compound of Formula (I), (I-a), (I-b), (I-c), (I-d), (I-e), (I-f), (I-g) or (I-h), Bⁿ is independently a natural, a modified, or an unnatural nucleobase. In some embodiments, Bⁿ is guanine. In some embodiments, Bⁿ is adenine. In some embodiments, Bⁿ is cytosine. In some embodiments, Bⁿ is uracil, In some embodiments, Bⁿ is thymine, In some embodiments, Bⁿ is hypoxanthine. In some embodiments, Bⁿ is purine.

In some embodiments of a compound of Formula (I), (I-a), (I-b), (I-c), (I-d), (I-e), (I-f), (I-g) or (I-h), at least one of B², B³, and Bⁿ is adenine. In some embodiments, at least one of B², B³, and Bⁿ is guanine. In some embodiments B² is adenine. In some embodiments, B³ is adenine.

In some embodiments of a compound of Formula (I), (I-a), (I-b), (I-c), (I-d), (I-e), (I-f), (I-g) or (I-h), Q¹ is —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH₂—. In some embodiments, Q¹ is —CH═CH—. In some embodiments, Q¹ is —CH₂O—. In some embodiments, Q¹ is —CH₂S—. In some embodiments, Q¹ is —CH₂CH₂—. In some embodiments, Q¹ is —CH₂CF₂—. In some embodiments, Q¹ is —CH₂NH₂—. In some embodiments, Q¹ is —CH₂NH(CH₃)—. In some embodiments, Q¹ is —CH₂N(C(═O)CH₃)—.

In some embodiments of a compound of Formula (I), (I-a), (I-b), (I-c), (I-d), (I-e), (I-f), (I-g) or (I-h), Q⁴ is —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q⁴ is —CH₂—. In some embodiments, Q⁴ is —CH═CH—. In some embodiments, Q⁴ is —CH₂O—. In some embodiments, Q⁴ is —CH₂S—. In some embodiments, Q⁴ is —CH₂CH₂—. In some embodiments, Q⁴ is —CH₂CF₂—. In some embodiments, Q⁴ is —CH₂NH₂—. In some embodiments, Q⁴ is —CH₂NH(CH₃)—. In some embodiments, Q⁴ is —CH₂N(C(═O)CH₃)—.

In some embodiments of a compound of Formula (I), (I-a), (I-b), (I-c), (I-d), (I-e), (I-f), (I-g) or (I-h), Q² is —O—, —S—, —CH₂—, —CF₂—, —NH—, —N(CH₃)—, or —N(C(═O)CH₃)—. In some embodiments, Q² is —O—. In some embodiments, Q² is —S—. In some embodiments, Q² is —CH₂—. In some embodiments, Q² is —CF₂—. In some embodiments, Q² is —NH—. In some embodiments, Q² is —N(CH₃)—. In some embodiments, Q² is —N(C(═O)CH₃).

In some embodiments of a compound of Formula (I), (I-a), (I-b), (I-c), (I-d), (I-e), (I-f), (I-g) or (I-h), Q³ is —O—, —S—, —CH₂—, —CF₂—, —NH—, —N(CH₃)—, or —N(C(═O)CH₃)—. In some embodiments, Q³ is —O—. In some embodiments, Q³ is —S—. In some embodiments, Q³ is —CH₂—. In some embodiments, Q³ is —CF₂—. In some embodiments, Q³ is —NH—. In some embodiments, Q³ is —N(CH₃)—. In some embodiments, Q³ is —N(C(═O)CH₃).

In some embodiments of a compound of Formula (I), (I-a), (I-b), (I-c), (I-d), (I-e), (I-f), (I-g) or (I-h), X¹ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X¹ is —OH. In some embodiments, X¹ is —SH. In some embodiments, X¹ is —O—. In some embodiments, X¹ is —S—. In some embodiments, X¹ is —NH₂. In some embodiments, X¹ is —NHCH₃. In some embodiments, X¹ is —NH(C(═O)CH₃). In some embodiments, X¹ is —CH₃. In some embodiments, X¹ is —CH₂CH₃. In some embodiments, X¹ is —CH₂CH₂CH₃. In some embodiments, X¹ is —CH(CH₃)₂. In some embodiments, X¹ is —OCH₃. In some embodiments, X¹ is —OCH₂CH₃.

In some embodiments of a compound of Formula (I), (I-a), (I-b), (I-c), (I-d), (I-e), (I-f), (I-g) or (I-h), X² is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X² is —OH. In some embodiments, X² is —SH. In some embodiments, X² is —O—. In some embodiments, X² is —S—. In some embodiments, X² is —NH₂. In some embodiments, X² is —NHCH₃. In some embodiments, X² is —NH(C(═O)CH₃). In some embodiments, X² is —CH₃. In some embodiments, X² is —CH₂CH₃. In some embodiments, X² is —CH₂CH₂CH₃. In some embodiments, X² is —CH(CH₃)₂. In some embodiments, X² is —OCH₃. In some embodiments, X² is —OCH₂CH₃.

In some embodiments of a compound of Formula (I), (I-a), (I-b), (I-c), (I-d), (I-e), (I-f), (I-g) or (I-h), X³ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X³ is —OH. In some embodiments, X³ is —SH. In some embodiments, X³ is —O—. In some embodiments, X³ is —S—. In some embodiments, X³ is —NH₂. In some embodiments, X³ is —NHCH₃. In some embodiments, X³ is —NH(C(═O)CH₃). In some embodiments, X³ is —CH₃. In some embodiments, X³ is —CH₂CH₃. In some embodiments, X³ is —CH₂CH₂CH₃. In some embodiments, X³ is —CH(CH₃)₂. In some embodiments, X³ is —OCH₃. In some embodiments, X³ is —OCH₂CH₃.

In some embodiments of a compound of Formula (I), (I-a), (I-b), (I-c), (I-d), (I-e), (I-f), (I-g) or (I-h), X⁴ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X⁴ is —OH. In some embodiments, X⁴ is —SH. In some embodiments, X⁴ is —O—. In some embodiments, X⁴ is —S—. In some embodiments, X⁴ is —NH₂. In some embodiments, X⁴ is —NHCH₃. In some embodiments, X⁴ is —NH(C(═O)CH₃). In some embodiments, X⁴ is —CH₃. In some embodiments, X⁴ is —CH₂CH₃. In some embodiments, X⁴ is —CH₂CH₂CH₃. In some embodiments, X⁴ is —CH(CH₃)₂. In some embodiments, X⁴ is —OCH₃. In some embodiments, X⁴ is —OCH₂CH₃.

In some embodiments of a compound of Formula (I), (I-a), (I-b), (I-c), (I-d), (I-e), (I-f), (I-g) or (I-h), Xⁿ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, Xⁿ is —OH. In some embodiments, Xⁿ is —SH. In some embodiments, Xⁿ is —O—. In some embodiments, Xⁿ is —S—. In some embodiments, Xⁿ is —NH₂. In some embodiments, Xⁿ is —NHCH₃. In some embodiments, Xⁿ is —NH(C(═O)CH₃). In some embodiments, Xⁿ is —CH₃. In some embodiments, Xⁿ is —CH₂CH₃. In some embodiments, Xⁿ is —CH₂CH₂CH₃. In some embodiments, Xⁿ is —CH(CH₃)₂. In some embodiments, Xⁿ is —OCH₃. In some embodiments, Xⁿ is —OCH₂CH₃.

In some embodiments of a compound of Formula (I), (I-a), (I-b), (I-c), (I-d), (I-e), (I-f), (I-g) or (I-h), Y¹ is ═O, ═S, ═NH, or ═NCH₃. In some embodiments, Y¹ is ═O. In some embodiments, Y¹ is ═S. In some embodiments, Y¹ is ═NH. In some embodiments, Y¹ is ═NHCH₃.

In some embodiments of a compound of Formula (I), (I-a), (I-b), (I-c), (I-d), (I-e), (I-f), (I-g) or (I-h), Y² is ═O, ═S, ═NH, or ═NCH₃. In some embodiments, Y² is ═O. In some embodiments, Y² is ═S. In some embodiments, Y² is ═NH. In some embodiments, Y² is ═NHCH₃.

In some embodiments of a compound of Formula (I), (I-a), (I-b), (I-c), (I-d), (I-e), (I-f), (I-g) or (I-h), Y³ is ═O, ═S, ═NH, or ═NCH₃. In some embodiments, Y³ is ═O. In some embodiments, Y³ is ═S. In some embodiments, Y³ is ═NH. In some embodiments, Y³ is ═NHCH₃.

In some embodiments of a compound of Formula (I), (I-a), (I-b), (I-c), (I-d), (I-e), (I-f), (I-g) or (I-h), Y⁴ is ═O, ═S, ═NH, or ═NCH₃. In some embodiments, Y⁴ is ═O. In some embodiments, Y⁴ is ═S. In some embodiments, Y⁴ is ═NH. In some embodiments, Y⁴ is ═NHCH₃.

In some embodiments of a compound of Formula (I), (I-a), (I-b), (I-c), (I-d), (I-e), (I-f), (I-g) or (I-h), Yⁿ is ═O, ═S, ═NH, or ═NCH₃. In some embodiments, Yⁿ is ═O. In some embodiments, Yⁿ is ═S. In some embodiments, Yⁿ is ═NH. In some embodiments, Yⁿ is ═NHCH₃.

In some embodiments of a compound of Formula (I), (I-a), (I-b), (I-c), (I-d), (I-e), (I-f), (I-g) or (I-h), A is —O—, —S—, —CH₂—, —NH—, —N(CH₃)— or —N(C(═O)CH₃)—. In some embodiments, A is —O—. In some embodiments, A is —S—. In some embodiments, A is —CH₂—. In some embodiments, A is —NH—. In some embodiments, A is —N(CH₃)—. In some embodiments, A is —N(C(═O)CH₃)—.

In some embodiments of a compound of Formula (I), (I-a), (I-b), (I-c), (I-d), (I-e), (I-f), (I-g) or (I-h), A¹ is —O—, —S—, —CH₂—, —NH—, —N(CH₃)— or —N(C(═O)CH₃)—. In some embodiments, A¹ is —O—. In some embodiments, A¹ is —S—. In some embodiments, A¹ is —CH₂—. In some embodiments, A¹ is —NH—. In some embodiments, A¹ is —N(CH₃)—. In some embodiments, A¹ is —N(C(═O)CH₃)—.

In some embodiments of a compound of Formula (I), (I-a), (I-b), (I-c), (I-d), (I-e), (I-f), (I-g) or (I-h), A² is —O—, —S—, —CH₂—, —NH—, —N(CH₃)— or —N(C(═O)CH₃)—. In some embodiments, A² is —O—. In some embodiments, A² is —S—. In some embodiments, A² is —CH₂—. In some embodiments, A² is —NH—. In some embodiments, A² is —N(CH₃)—. In some embodiments, A² is —N(C(═O)CH₃)—.

In some embodiments of a compound of Formula (I), (I-a), (I-b), (I-c), (I-d), (I-e), (I-f), (I-g) or (I-h), p is 0, 1, 2, 3, 4, 5 or 6. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments of a compound of Formula (I), at least one of X¹, X², X³, X⁴, and Xⁿ is —SH.

In some embodiments of a compound of Formula (I), at least one of X¹, X², X³, X⁴, and X is —S—.

In some embodiments of a compound of Formula (I), at least one of Y¹, Y², Y³, Y⁴, and Yⁿ is ═S.

In some embodiments of a compound of Formula (I), at least one of A, A¹, and A² is —S—.

In some embodiments of a compound of Formula (I), at least one of Z¹, Z², Z³, Z⁴, and Zⁿ is —OCH₃. In some embodiments, Z³ is —OCH₃. In some embodiments, Z³ and Z¹ are —OCH₃. In some embodiments of a compound of Formula (I), at least one of Z¹, Z², Z³, Z⁴, and Zⁿ is —OH. In some embodiments, Z¹, Z², and Z⁴ are —OH. In some embodiments, Z² and Z⁴ are —OH.

In some embodiments of a compound of Formula (I), at least one of Q¹, Q², Q³ and Q⁴ is —OCH₃. In some embodiments of a compound of Formula (I), at least one of Q¹, Q², Q³, and Q⁴ is —O—. In some embodiments, Q¹ and Q⁴ are —OCH₃. In some embodiments, Q² and Q³ are —O—.

In some embodiments of a compound o Formula (I), at least one of Y¹, Y², Y³, Y⁴ and Yⁿ is ═O. In some embodiments, Y¹, Y², Y³, and Y⁴ is are ═O. In some embodiments, Y¹, Y², Y³, Y⁴, and Yⁿ is ═S. In some embodiments, Y² is ═S. In some embodiments, Y⁴ is ═S.

In some embodiments of a compound of Formula (I), at least one of X¹, X², X³, X⁴, and Xⁿ is —O—. In Some embodiments, X¹, X², X³, and X⁴ are —O—. In some embodiments, X¹ is —S—. In some embodiments, X² is —S—. In some embodiments, X³ is —S—. In some embodiments, X⁴ is —S—.

In some embodiments of a compound of Formula (I), In some embodiments of a compound of Formula (I), at least one of A, A¹, and A² is —O—. In some embodiments, A, A¹, and A² are —O—.

In one aspect, described herein is a mRNA sequence having a 5′-end region motif of Motif (I′):

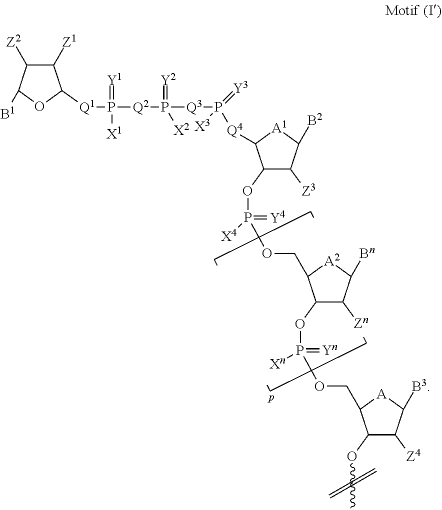

wherein
B¹ is

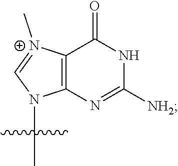 [SMILES: *n1c[n+](C)c2c(=O)[nH]c(N)nc21]

each B², B³, and Bⁿ is independently a natural, a modified, or an unnatural nucleobase;
each Z¹ and Z² is independently hydrogen, fluorine, —OH, —SH, —CH₃, —CH₂CH₃, —OCH₃, —OCH₂CH₃, —SCH₃, —NH₂, NHCH₃, or NHC(═O)CH₃;
each Z³, Z⁴, and Zⁿ is independently hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6,
provided that (i) Z¹, Z², and Z³ are hydrogen; (ii) Q¹ and Q⁴ are —CH₂O—; (iii) Q² and Q³ are oxygen; (iv) at least one of X¹, X², X³, X⁴, and Xⁿ is —SH or —S—; (v) at least one of Y¹, Y², Y³, Y⁴, and Yⁿ is ═S; or (vi) at least one of A, A¹, and A² is —S—.

In some embodiments, the mRNA sequence having a 5′-end region motif has a structure of Motif (I′-a):

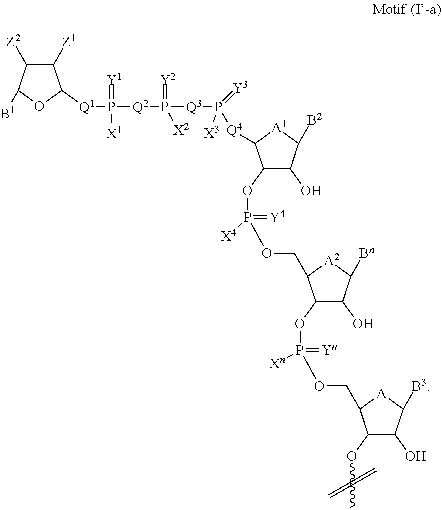

In some embodiments, the mRNA sequence having a 5′-end region motif has a structure of Motif (I′-b):

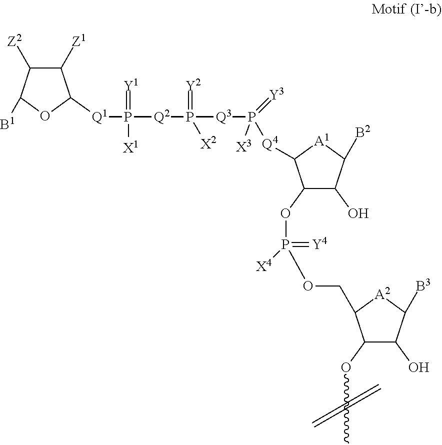

In some embodiments, the mRNA sequence having a 5′-end region motif has a structure of Motif (I′-c):

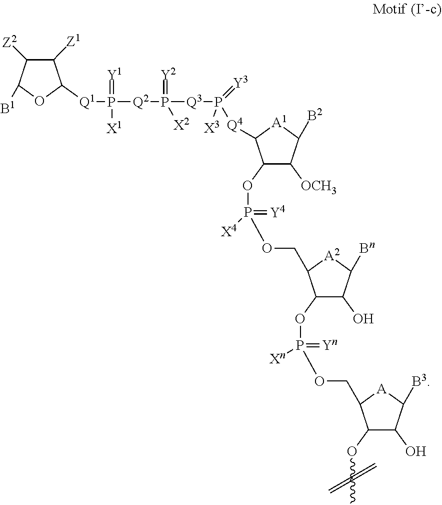

In some embodiments, the mRNA sequence having a 5′-end region motif has a structure of Motif (I′-d):

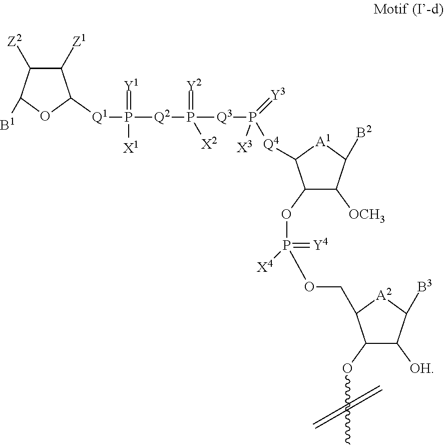

In some embodiments, the mRNA sequence having a 5′-end region motif has a structure of Motif (I′-e):

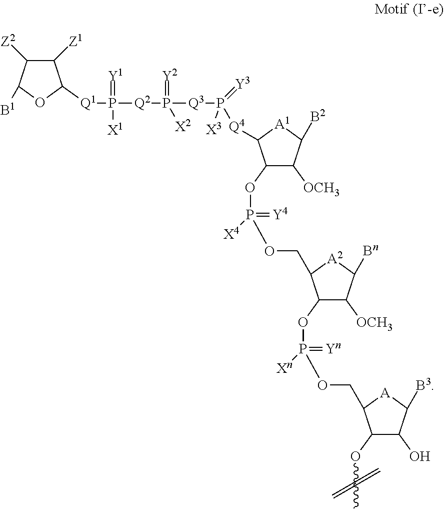

In some embodiments, the mRNA sequence having a 5′-end region motif has a structure of Motif (I′-f):

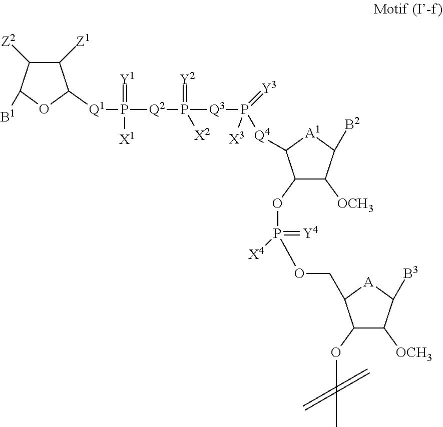

In some embodiments, the mRNA sequence having a 5′-end region motif has a structure of Motif (I′-g):

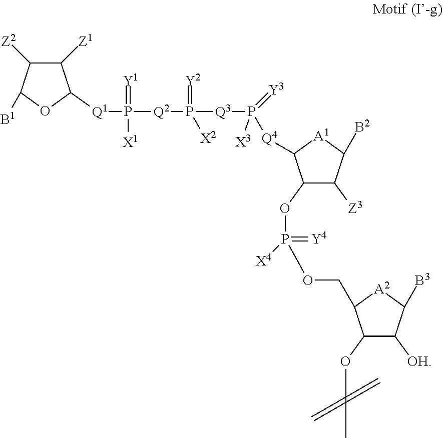

In some embodiments, the mRNA sequence having a 5′-end region motif has a structure of Motif (I′-h):

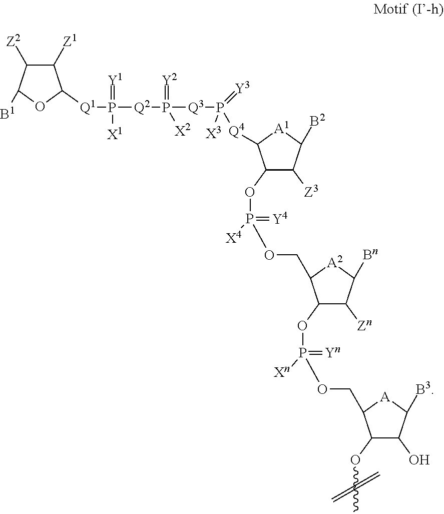

In some embodiments of a compound of Motif (I′), (I′-a), (I′-b), (I′-c), (I′-d), (I′-e), (I′-f), (I′-g) or (I′-h), Z¹ is hydrogen, fluorine, —OH, —SH, —CH₃, —CH₂CH₃, —OCH₃, —OCH₂CH₃, —SCH₃, —NH₂, NHCH₃, or NHC(═O)CH₃. In some embodiments, Z¹ is hydrogen. In some embodiments, Z¹ is F. In some embodiments, Z¹ is —OH. In some embodiments, Z¹ is —SH. In some embodiments, Z¹ is —CH₃. In some embodiments, Z¹ is —CH₂CH₃. In some embodiments, Z¹ is —OCH₃. In some embodiments, Z¹ is —OCH₂CH₃. In some embodiments, Z¹ is —SCH₃. In some embodiments, Z¹ is —NH₂. In some embodiments, Z¹ is NHCH₃. In some embodiments, Z¹ is NHC(═O)CH₃.

In some embodiments of a compound of Motif (I′), (I′-a), (I′-b), (I′-a), (I′-d), (I′-e), (I′-f), (I′-g) or (I′-h), Z² is hydrogen, fluorine, —OH, —SH, —CH₃, —CH₂CH₃, —OCH₃, —OCH₂CH₃, —SCH₃, —NH₂, NHCH₃, or NHC(═O)CH₃. In some embodiments, Z² is hydrogen. In some embodiments, Z² is F. In some embodiments, Z² is —OH. In some embodiments, Z² is —SH. In some embodiments, Z² is —CH₃. In some embodiments, Z² is —CH₂CH₃. In some embodiments, Z² is —OCH₃. In some embodiments, Z² is —OCH₂CH₃. In some embodiments, Z² is —SCH₃. In some embodiments, Z² is —NH₂. In some embodiments, Z² is NHCH₃. In some embodiments, Z² is NHC(═O)CH₃.

In some embodiments of a compound of Motif (I′), (I′-a), (I′-b), (I′-c), (I′-d), (I′-e), (I′-f), (I′-g) or (I′-h), Z³ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z³ is hydrogen. In some embodiments, Z³ is fluorine. In some embodiments, Z³ is —OH. In some embodiments, Z³ is —SH. In some embodiments, Z³ is —CH₃. In some embodiments, Z³ is —CH₂CH₃. In some embodiments, Z³ is —OCH₂OCH₃. In some embodiments, Z³ is —OCH₂CH₂CH₃. In some embodiments, Z³ is —OCH(CH₃)₂. In some embodiments, Z³ is —SCH₃. In some embodiments, Z³ is —OCH₂CH₂OCH₃.

In some embodiments of a compound of Motif (I′), (I′-a), (I′-b), (I′-c), (I′-d), (I′-e), (I′-f), (I′-g) or (I′-h), Z⁴ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z⁴ is hydrogen. In some embodiments, Z⁴ is fluorine. In some embodiments, Z⁴ is —OH. In some embodiments, Z⁴ is —SH. In some embodiments, Z⁴ is —CH₃. In some embodiments, Z⁴ is —CH₂CH₃. In some embodiments, Z⁴ is —OCH₂OCH₃. In some embodiments, Z⁴ is —OCH₂CH₂CH₃. In some embodiments, Z⁴ is —OCH(CH₃)₂. In some embodiments, Z⁴ is —SCH₃. In some embodiments, Z⁴ is —OCH₂CH₂OCH₃.

In some embodiments of a compound of Motif (I′), (I′-a), (I′-b), (I′-c), (I′-d), (I′-e), (I′-f), (I′-g) or (I′-h), Zⁿ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Zⁿ is hydrogen. In some embodiments, Zⁿ is fluorine. In some embodiments, Zⁿ is —OH. In some embodiments, Zⁿ is —SH. In some embodiments, Zⁿ is —CH₃. In some embodiments, Zⁿ is —CH₂CH₃. In some embodiments, Zⁿ is —OCH₂OCH₃. In some embodiments, Zⁿ is —OCH₂CH₂CH₃. In some embodiments, Zⁿ is —OCH(CH₃)₂. In some embodiments, Zⁿ is —SCH₃. In some embodiments, Zⁿ is —OCH₂CH₂OCH₃.

In some embodiments of a compound of Motif (I′), (I′-a), (I′-b), (I′-c), (I′-d), (I′-e), (I′-f), (I′-g) or (I′-h), B² is independently a natural, a modified, or an unnatural nucleobase. In some embodiments B² is guanine. In some embodiments B² is adenine. In some embodiments, B² is cytosine. In some embodiments, B² is uracil, In some embodiments, B² is thymine, In some embodiments, B² is hypoxanthine. In some embodiments, B² is purine.

In some embodiments of a compound of Motif (I′), (I′-a), (I′-b), (I′-c), (I′-d), (I′-e), (I′-f), (I′-g) or (I′-h), B³ is independently a natural, a modified, or an unnatural nucleobase. In some embodiments, B³ is guanine. In some embodiments, B³ is adenine. In some embodiments, B³ is cytosine. In some embodiments, B³ is uracil, In some embodiments, B³ is thymine, In some embodiments, B³ is hypoxanthine. In some embodiments, B³ is purine.

In some embodiments of a compound of Motif (I′), (I′-a), (I′-b), (I′-c), (I′-d), (I′-e), (I′-f), (I′-g) or (I′-h), Bⁿ is independently a natural, a modified, or an unnatural nucleobase. In some embodiments, Bⁿ is guanine. In some embodiments, Bⁿ is adenine. In some embodiments, Bⁿ is cytosine. In some embodiments, Bⁿ is uracil, In some embodiments, Bⁿ is thymine, In some embodiments, Bⁿ is hypoxanthine. In some embodiments, Bⁿ is purine.

In some embodiments of a compound of Motif (I′), (I′-a), (I′-b), (I′-c), (I′-d), (I′-e), (I′-f), (I′-g) or (I′-h), at least one of B², B³, and Bⁿ is adenine. In some embodiments, at least one of B², B³, and Bⁿ is guanine. In some embodiments B² is adenine. In some embodiments, B³ is adenine.

In some embodiments of a compound of Motif (I′), (I′-a), (I′-b), (I′-c), (I′-d), (I′-e), (I′-f), (I′-g) or (I′-h), Q¹ is —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH₂—. In some embodiments, Q¹ is —CH═CH—. In some embodiments, Q¹ is —CH₂O—. In some embodiments, Q¹ is —CH₂S—. In some embodiments, Q¹ is —CH₂CH₂—. In some embodiments, Q¹ is —CH₂CF₂—. In some embodiments, Q¹ is —CH₂NH₂—. In some embodiments, Q¹ is —CH₂NH(CH₃)—. In some embodiments, Q¹ is —CH₂N(C(═O)CH₃)—.

In some embodiments of a compound of Motif (I′), (I′-a), (I′-b), (I′-c), (I′-d), (I′-e), (I′-f), (I′-g) or (I′-h), Q⁴ is —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q⁴ is —CH₂—. In some embodiments, Q⁴ is —CH═CH—. In some embodiments, Q⁴ is —CH₂O—. In some embodiments, Q⁴ is —CH₂S—. In some embodiments, Q⁴ is —CH₂CH₂—. In some embodiments, Q⁴ is —CH₂CF₂—. In some embodiments, Q⁴ is —CH₂NH₂—. In some embodiments, Q⁴ is —CH₂NH(CH₃)—. In some embodiments, Q⁴ is —CH₂N(C(═O)CH₃)—.

In some embodiments of a compound of Motif (I′), (I′-a), (I′-b), (I′-c), (I′-d), (I′-e), (I′-f), (I′-g) or (I′-h), Q² is —O—, —S—, —CH₂—, —CF₂—, —NH—, —N(CH₃)—, or —N(C(═O)CH₃)—. In some embodiments, Q² is —O—. In some embodiments, Q² is —S—. In some embodiments, Q² is —CH₂—. In some embodiments, Q² is —CF₂—. In some embodiments, Q² is —NH—. In some embodiments, Q² is —N(CH₃)—. In some embodiments, Q² is —N(C(═O)CH₃).

In some embodiments of a compound of Motif (I′), (I′-a), (I′-b), (I′-c), (I′-d), (I′-e), (I′-f), (I′-g) or (I′-h), Q³ is —O—, —S—, —CH₂—, —CF₂—, —NH—, —N(CH₃)—, or —N(C(═O)CH₃)—. In some embodiments, Q³ is —O—. In some embodiments, Q³ is —S—. In some embodiments, Q³ is —CH₂—. In some embodiments, Q³ is —CF₂—. In some embodiments, Q³ is —NH—. In some embodiments, Q³ is —N(CH₃)—. In some embodiments, Q³ is —N(C(═O)CH₃).

In some embodiments of a compound of Motif (I′), (I′-a), (I′-b), (I′-c), (I′-d), (I′-e), (I′-f), (I′-g) or (I′-h), X¹ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X¹ is —OH. In some embodiments, X¹ is —SH. In some embodiments, X¹ is —O—. In some embodiments, X¹ is —S—. In some embodiments, X¹ is —NH₂. In some embodiments, X¹ is —NHCH₃. In some embodiments, X¹ is —NH(C(═O)CH₃). In some embodiments, X¹ is —CH₃. In some embodiments, X¹ is —CH₂CH₃. In some embodiments, X¹ is —CH₂CH₂CH₃. In some embodiments, X¹ is —CH(CH₃)₂. In some embodiments, X¹ is —OCH₃. In some embodiments, X¹ is —OCH₂CH₃.

In some embodiments of a compound of Motif (I′), (I′-a), (I′-b), (I′-c), (I′-d), (I′-e), (I′-f), (I′-g) or (I′-h), X² is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X² is —OH. In some embodiments, X² is —SH. In some embodiments, X² is —O—. In some embodiments, X² is —S—. In some embodiments, X² is —NH₂. In some embodiments, X² is —NHCH₃. In some embodiments, X² is —NH(C(═O)CH₃). In some embodiments, X² is —CH₃. In some embodiments, X² is —CH₂CH₃. In some embodiments, X² is —CH₂CH₂CH₃. In some embodiments, X² is —CH(CH₃)₂. In some embodiments, X² is —OCH₃. In some embodiments, X² is —OCH₂CH₃.

In some embodiments of a compound of Motif (I′), (I′-a), (I′-b), (I′-c), (I′-d), (I′-e), (I′-f), (I′-g) or (I′-h), X³ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X³ is —OH. In some embodiments, X³ is —SH. In some embodiments, X³ is —O—. In some embodiments, X³ is —S—. In some embodiments, X³ is —NH₂. In some embodiments, X³ is —NHCH₃. In some embodiments, X³ is —NH(C(═O)CH₃). In some embodiments, X³ is —CH₃. In some embodiments, X³ is —CH₂CH₃. In some embodiments, X³ is —CH₂CH₂CH₃. In some embodiments, X³ is —CH(CH₃)₂. In some embodiments, X³ is —OCH₃. In some embodiments, X³ is —OCH₂CH₃.

In some embodiments of a compound of Motif (I′), (I′-a), (I′-b), (I′-c), (I′-d), (I′-e), (I′-f), (I′-g) or (I′-h), X⁴ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X⁴ is —OH. In some embodiments, X⁴ is —SH. In some embodiments, X⁴ is —O⁻. In some embodiments, X⁴ is —S⁻. In some embodiments, X⁴ is —NH₂. In some embodiments, X⁴ is —NHCH₃. In some embodiments, X⁴ is —NH(C(═O)CH₃). In some embodiments, X⁴ is —CH₃. In some embodiments, X⁴ is —CH₂CH₃. In some embodiments, X⁴ is —CH₂CH₂CH₃. In some embodiments, X⁴ is —CH(CH₃)₂. In some embodiments, X⁴ is —OCH₃. In some embodiments, X⁴ is —OCH₂CH₃.

In some embodiments of a compound of Motif (I′), (I′-a), (I′-b), (I′-c), (I′-d), (I′-e), (I′-f), (I′-g) or (I′-h), Xⁿ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, Xⁿ is —OH. In some embodiments, Xⁿ is —SH. In some embodiments, Xⁿ is —O⁻. In some embodiments, Xⁿ is —S⁻. In some embodiments, Xⁿ is —NH₂. In some embodiments, Xⁿ is —NHCH₃. In some embodiments, Xⁿ is —NH(C(═O)CH₃). In some embodiments, Xⁿ is —CH₃. In some embodiments, Xⁿ is —CH₂CH₃. In some embodiments, Xⁿ is —CH₂CH₂CH₃. In some embodiments, Xⁿ is —CH(CH₃)₂. In some embodiments, Xⁿ is —OCH₃. In some embodiments, Xⁿ is —OCH₂CH₃.

In some embodiments of a compound of Motif (I′), (I′-a), (I′-b), (I′-c), (I′-d), (I′-e), (I′-f), (I′-g) or (I′-h), Y¹ is ═O, ═S, ═NH, or ═NCH₃. In some embodiments, Y¹ is ═O. In some embodiments, Y¹ is ═S. In some embodiments, Y¹ is ═NH. In some embodiments, Y¹ is ═NHCH₃.

In some embodiments of a compound of Motif (I′), (I′-a), (I′-b), (I′-c), (I′-d), (I′-e), (I′-f), (I′-g) or (I′-h), Y² is ═O, ═S, ═NH, or ═NCH₃. In some embodiments, Y² is ═O. In some embodiments, Y² is ═S. In some embodiments, Y² is ═NH. In some embodiments, Y² is ═NHCH₃.

In some embodiments of a compound of Motif (I′), (I′-a), (I′-b), (I′-c), (I′-d), (I′-e), (I′-f), (I′-g) or (I′-h), Y³ is ═O, ═S, ═NH, or ═NCH₃. In some embodiments, Y³ is ═O. In some embodiments, Y³ is ═S. In some embodiments, Y³ is ═NH. In some embodiments, Y³ is ═NHCH₃.

In some embodiments of a compound of Motif (I′), (I′-a), (I′-b), (I′-c), (I′-d), (I′-e), (I′-f), (I′-g) or (I′-h), Y⁴ is ═O, ═S, ═NH, or ═NCH₃. In some embodiments, Y⁴ is ═O. In some embodiments, Y⁴ is ═S. In some embodiments, Y⁴ is ═NH. In some embodiments, Y⁴ is ═NHCH₃.

In some embodiments of a compound of Motif (I′), (I′-a), (I′-b), (I′-c), (I′-d), (I′-e), (I′-f), (I′-g) or (I′-h), Yⁿ is ═O, ═S, ═NH, or ═NCH₃. In some embodiments, Yⁿ is ═O. In some embodiments, Yⁿ is ═S. In some embodiments, Yⁿ is ═NH. In some embodiments, Yⁿ is ═NHCH₃.

In some embodiments of a compound of Motif (I′), (I′-a), (I′-b), (I′-c), (I′-d), (I′-e), (I′-f), (I′-g) or (I′-h), A is —O—, —S—, —CH₂—, —NH—, —N(CH₃)— or —N(C(═O)CH₃)—. In some embodiments, A is —O—. In some embodiments, A is —S—. In some embodiments, A is —CH₂—. In some embodiments, A is —NH—. In some embodiments, A is —N(CH₃)—. In some embodiments, A is —N(C(═O)CH₃)—.

In some embodiments of a compound of Motif (I′), (I′-a), (I′-b), (I′-c), (I′-d), (I′-e), (I′-f), (I′-g) or (I′-h), A¹ is —O—, —S—, —CH₂—, —NH—, —N(CH₃)— or —N(C(═O)CH₃)—. In some embodiments, A¹ is —O—. In some embodiments, A¹ is —S—. In some embodiments, A¹ is —CH₂—. In some embodiments, A¹ is —NH—. In some embodiments, A¹ is —N(CH₃)—. In some embodiments, A¹ is —N(C(═O)CH₃)—.

In some embodiments of a compound of Motif (I′), (I′-a), (I′-b), (I′-c), (I′-d), (I′-e), (I′-f), (I′-g) or (I′-h), A² is —O—, —S—, —CH₂—, —NH—, —N(CH₃)— or —N(C(═O)CH₃)—. In some embodiments, A² is —O—. In some embodiments, A² is —S—. In some embodiments, A² is —CH₂—. In some embodiments, A² is —NH—. In some embodiments, A² is —N(CH₃)—. In some embodiments, A² is —N(C(═O)CH₃)—.

In some embodiments of a compound of Motif (I′), (I′-a), (I′-b), (I′-c), (I′-d), (I′-e), (I′-f), (I′-g) or (I′-h), p is 0, 1, 2, 3, 4, 5 or 6. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments of a compound of Motif (I′), at least one of X¹, X², X³, X⁴, and Xⁿ is —SH.

In some embodiments of a compound of Motif (I′), at least one of X¹, X², X³, X⁴, and X is —S—.

In some embodiments of a compound of Motif (I′), at least one of Y¹, Y², Y³, Y⁴ and Yⁿ is ═S.

In some embodiments of a compound of Motif (I′), at least one of A, A¹, and A² is —S—.

In some embodiments of a compound of Motif (I′), at least one of Z¹, Z², Z³, Z⁴, and Zⁿ is —OCH₃. In some embodiments, Z³ is —OCH₃. In some embodiments, Z³ and Z¹ are —OCH₃. In some embodiments of a compound of Motif (I′), at least one of Z¹, Z², Z³, Z⁴, and Zⁿ is —OH. In some embodiments, Z¹, Z², and Z⁴ are —OH. In some embodiments, Z² and Z⁴ are —OH.

In some embodiments of a compound of Motif (I′), at least one of Q¹, Q², Q³, and Q⁴ is —OCH₃. In some embodiments of a compound of Formula (I), at least one of Q¹, Q², Q³, and Q⁴ is —O—. In some embodiments, Q¹ and Q⁴ are —OCH₃. In some embodiments, Q² and Q³ are —O—.

In some embodiments of a compound o Motif (I′), at least one of Y¹, Y², Y³, Y⁴ and Yⁿ is ═O. In some embodiments, Y¹, Y², Y³, and Y⁴ is are ═O. In some embodiments, Y¹, Y², Y³, Y⁴, and Yⁿ is ═S. In some embodiments, Y² is ═S. In some embodiments, Y⁴ is ═S.

In some embodiments of a compound of Motif (I′), at least one of X¹, X², X³, X⁴, and Xⁿ is —O⁻. In Some embodiments, X¹, X², X³, and X⁴ are —O⁻. In some embodiments, X¹ is —S⁻. In some embodiments, X² is —S⁻. In some embodiments, X³ is —S⁻. In some embodiments, X⁴ is —S⁻.

In some embodiments of a compound of Motif (I′), In some embodiments of a compound of Motif (I′), at least one of A, A¹, and A² is —O⁻. In some embodiments, A, A¹, and A² are —O⁻.

In one aspect, described herein is an mRNA sequence initiator comprising a compound of Formula (II) or a salt or solvate thereof:

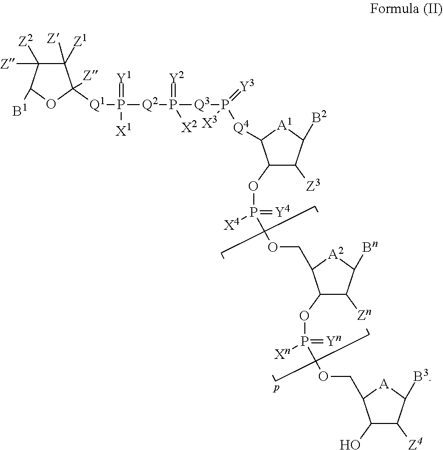

wherein
B¹ is

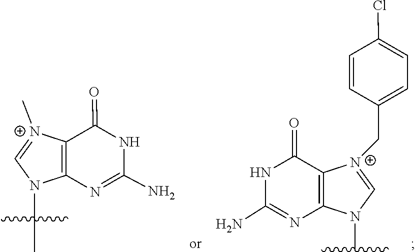 [SMILES: *n1c[n+](C)c2c(=O)[nH]c(N)nc21 ; *n1c[n+](Cc2ccc(Cl)cc2)c2c(=O)[nH]c(N)nc21]

each B², B³, and Bⁿ is independently a natural, a modified, or an unnatural nucleobase;
each Z¹ and Z′ is independently is hydrogen, fluorine, —OH, —SH, —CH₃, —CH₂CH₃, —OCH₃, —NH(CH₃), —NH₂, —NH(C(═O)CH₃), or —SCH₃;
each Z² and Zⁿ is independently fluorine, —OH, —SH, —CH₃, —CH₂CH₃, —OCH₃, —SCH₃, —OCH₂CH₃, —NH₂, NHCH₃, or NHC(═O)CH₃;
Z′″ is hydrogen, fluorine, —CH₃, —CH₂CH₃, —OCH₃, or —OCH₂CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u) is a salt. In some embodiments, the salt is an alkali metal salt. In some embodiments, the salt is a sodium salt. In some embodiments, the salt is an ammonium salt.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), B² independently a natural, a modified, or an unnatural nucleobase. In some embodiments, B² is adenine. In some embodiments, B² is guanine. In some embodiments, B² is cytosine. In some embodiments, B² is uracil, In some embodiments, B² is thymine, In some embodiments, B² is hypoxanthine. In some embodiments, B² is purine.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), B³ independently a natural, a modified, or an unnatural nucleobase. In some embodiments, B³ is adenine. In some embodiments, B³ is guanine. In some embodiments, B³ is cytosine. In some embodiments, B³ is uracil, In some embodiments, B³ is thymine, In some embodiments, B³ is hypoxanthine. In some embodiments, B³ is purine.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Bⁿ independently a natural, a modified, or an unnatural nucleobase. In some embodiments, Bⁿ is adenine. In some embodiments, Bⁿ is guanine. In some embodiments, Bⁿ is cytosine. In some embodiments, Bⁿ is uracil, In some embodiments, Bⁿ is thymine, In some embodiments, Bⁿ is hypoxanthine. In some embodiments, Bⁿ is purine.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Z¹ is hydrogen, fluorine, —OH, —SH, —CH₃, —CH₂CH₃, —OCH₃, —NH(CH₃), —NH₂, —NH(C(═O)CH₃), or —SCH₃. In some embodiments, Z¹ is hydrogen. In some embodiments, Z¹ is fluorine. In some embodiments, Z¹ is —OH. In some embodiments, Z¹ is —SH. In some embodiments, Z¹ is —CH₃. In some embodiments, Z¹ is —CH₂CH₃. In some embodiments, Z¹ is —OCH₃. In some embodiments, Z¹ is —NH(CH₃), In some embodiments, Z¹ is —NH₂—. In some embodiments, Z¹ is —NH(C(═O)CH₃). In some embodiments, Z¹ is —SCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Z′ is hydrogen, fluorine, —OH, —SH, —CH₃, —CH₂CH₃, —OCH₃, —NH(CH₃), —NH₂, —NH(C(═O)CH₃), or —SCH₃. In some embodiments, Z′ is hydrogen. In some embodiments, Z′ is fluorine. In some embodiments, Z′ is —OH. In some embodiments, Z′ is —SH. In some embodiments, Z′ is —CH₃. In some embodiments, Z′ is —CH₂CH₃. In some embodiments, Z′ is —OCH₃. In some embodiments, Z′ is —NH(CH₃), In some embodiments, Z′ is —NH₂—. In some embodiments, Z′ is —NH(C(═O)CH₃). In some embodiments, Z′ is —SCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Z² is fluorine, —OH, —SH, —CH₃, —CH₂CH₃, —OCH₃, —SCH₃, —OCH₂CH₃, —NH₂, NHCH₃, or NHC(═O)CH₃. In some embodiments, Z² is fluorine. In some embodiments, Z² is —OH. In some embodiments, Z² is —SH. In some embodiments, Z² is —CH₃. In some embodiments, Z² is —CH₂CH₃. In some embodiments, Z² is —OCH₃. In some embodiments, Z² is —SCH₃. In some embodiments, Z² is —OCH₂CH₃. In some embodiments, Z² is —NH₂. In some embodiments, Z² is NHCH₃. In some embodiments, Z² is NHC(═O)CH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (11-s), (II-t), or (II-u), Zⁿ is fluorine, —OH, —SH, —CH₃, —CH₂CH₃, —OCH₃, —SCH₃, —OCH₂CH₃, —NH₂, NHCH₃, or NHC(═O)CH₃. In some embodiments, Zⁿ is fluorine. In some embodiments, Zⁿ is —OH. In some embodiments, Zⁿ is —SH. In some embodiments, Zⁿ is —CH₃. In some embodiments, Zⁿ is —CH₂CH₃. In some embodiments, Zⁿ is —OCH₃. In some embodiments, Zⁿ is —SCH₃. In some embodiments, Zⁿ is —OCH₂CH₃. In some embodiments, Zⁿ is —NH₂. In some embodiments, Zⁿ is NHCH₃. In some embodiments, Zⁿ is NHC(═O)CH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Z′″ is hydrogen, fluorine, —CH₃, —CH₂CH₃, —OCH₃, or —OCH₂CH₃. In some embodiments, Z′″ is hydrogen. In some embodiments, Z′″ is fluorine. In some embodiments, Z′″ is —CH₃. In some embodiments, Z′″ is —CH₂CH₃. In some embodiments, Z′″ is —OCH₃. In some embodiments, Z′″ is —OCH₂CH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Z³ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z³ is hydrogen. In some embodiments, Z³ is fluorine. In some embodiments, Z³ is —OH. In some embodiments, Z³ is —CH₃. In some embodiments, Z³ is —CH₂CH₃. In some embodiments, Z³ is —OCH₃. In some embodiments, Z³ is —NH₂. In some embodiments, Z³ is —NHCH₃. In some embodiments, Z³ is —NH(C(═O)CH₃). In some embodiments, Z³ is —OCH₂CH₃. In some embodiments, Z³ is —OCH₂OCH₃. In some embodiments, Z³ is —OCH₂CH₂CH₃. In some embodiments, Z³ is —OCH(CH₃)₂. In some embodiments, Z³ is —SCH₃. In some embodiments, Z³ is —OCH₂CH₂OCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Z⁴ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z⁴ is hydrogen. In some embodiments, Z⁴ is fluorine. In some embodiments, Z⁴ is —OH. In some embodiments, Z⁴ is —CH₃. In some embodiments, Z⁴ is —CH₂CH₃. In some embodiments, Z⁴ is —OCH₃. In some embodiments, Z⁴ is —NH₂. In some embodiments, Z⁴ is —NHCH₃. In some embodiments, Z⁴ is —NH(C(═O)CH₃). In some embodiments, Z⁴ is —OCH₂CH₃. In some embodiments, Z⁴ is —OCH₂OCH₃. In some embodiments, Z⁴ is —OCH₂CH₂CH₃. In some embodiments, Z⁴ is —OCH(CH₃)₂. In some embodiments, Z⁴ is —SCH₃. In some embodiments, Z⁴ is —OCH₂CH₂OCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Zⁿ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Zⁿ is hydrogen. In some embodiments, Zⁿ is fluorine. In some embodiments, Zⁿ is —OH. In some embodiments, Zⁿ is —CH₃. In some embodiments, Zⁿ is —CH₂CH₃. In some embodiments, Zⁿ is —OCH₃. In some embodiments, Zⁿ is —NH₂. In some embodiments, Zⁿ is —NHCH₃. In some embodiments, Zⁿ is —NH(C(═O)CH₃). In some embodiments, Zⁿ is —OCH₂CH₃. In some embodiments, Zⁿ is —OCH₂OCH₃. In some embodiments, Zⁿ is —OCH₂CH₂CH₃. In some embodiments, Zⁿ is —OCH(CH₃)₂. In some embodiments, Zⁿ is —SCH₃. In some embodiments, Zⁿ is —OCH₂CH₂OCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Q¹ is —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH═CH—. In some embodiments, Q¹ is —CH₂—. In some embodiments, Q¹ is —CH₂O—. In some embodiments, Q¹ is —CH₂S—. In some embodiments, Q¹ is —CH₂CH₂—. In some embodiments, Q¹ is —CH₂CF₂—. In some embodiments, Q¹ is —CH₂NH₂—. In some embodiments, Q¹ is —CH₂NH(CH₃)—. In some embodiments, Q¹ is —CH₂N(C(═O)CH₃)—.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Q⁴ is —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH═CH—. In some embodiments, Q⁴ is —CH₂—. In some embodiments, Q⁴ is —CH₂O—. In some embodiments, Q⁴ is —CH₂S—. In some embodiments, Q⁴ is —CH₂CH₂—. In some embodiments, Q⁴ is —CH₂CF₂—. In some embodiments, Q⁴ is —CH₂NH₂—. In some embodiments, Q⁴ is —CH₂NH(CH₃)—. In some embodiments, Q⁴ is —CH₂N(C(═O)CH₃)—.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Q² is —O—, —S—, —CH₂—, —CF₂—, —NH—, —N(CH₃)—, or —N(C(═O)CH₃)—. In some embodiments Q² is —O—. In some embodiments Q² is —S—. In some embodiments Q² is —CH₂—. In some embodiments Q² is —CF₂—. In some embodiments Q² is —NH—. In some embodiments Q² is —N(CH₃)—. In some embodiments Q² is —N(C(═O)CH₃)—.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Q³ is —O—, —S—, —CH₂—, —CF₂—, —NH—, —N(CH₃)—, or —N(C(═O)CH₃)—. In some embodiments Q³ is —O—. In some embodiments Q³ is —S—. In some embodiments Q³ is —CH₂—. In some embodiments Q³ is —CF₂—. In some embodiments Q³ is —NH—. In some embodiments Q³ is —N(CH₃)—. In some embodiments Q³ is —N(C(═O)CH₃)—.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), X¹ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X¹ is —OH. In some embodiments, X¹ is —SH. In some embodiments, X¹ is —O⁻. In some embodiments, X¹ is —S⁻. In some embodiments, X¹ is —NH₂. In some embodiments, X¹ is —NHCH₃. In some embodiments, X¹ is —NH(C(═O)CH₃). In some embodiments, X¹ is —CH₃. In some embodiments, X¹ is —CH₂CH₃. In some embodiments, X¹ is —CH₂CH₂CH₃. In some embodiments, X¹ is —CH(CH₃)₂. In some embodiments, X¹ is —OCH₃. In some embodiments, X¹ is —OCH₂CH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), X² is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X² is —OH. In some embodiments, X² is —SH. In some embodiments, X² is —O⁻. In some embodiments, X² is —S⁻. In some embodiments, X² is —NH₂. In some embodiments, X² is —NHCH₃. In some embodiments, X² is —NH(C(═O)CH₃). In some embodiments, X² is —CH₃. In some embodiments, X² is —CH₂CH₃. In some embodiments, X² is —CH₂CH₂CH₃. In some embodiments, X² is —CH(CH₃)₂. In some embodiments, X² is —OCH₃. In some embodiments, X² is —OCH₂CH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), X³ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X³ is —OH. In some embodiments, X³ is —SH. In some embodiments, X³ is —O⁻. In some embodiments, X³ is —S⁻. In some embodiments, X³ is —NH₂. In some embodiments, X³ is —NHCH₃. In some embodiments, X³ is —NH(C(═O)CH₃). In some embodiments, X³ is —CH₃. In some embodiments, X³ is —CH₂CH₃. In some embodiments, X³ is —CH₂CH₂CH₃. In some embodiments, X³ is —CH(CH₃)₂. In some embodiments, X³ is —OCH₃. In some embodiments, X³ is —OCH₂CH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), X⁴ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X⁴ is —OH. In some embodiments, X⁴ is —SH. In some embodiments, X⁴ is —O⁻. In some embodiments, X⁴ is —S⁻. In some embodiments, X⁴ is —NH₂. In some embodiments, X⁴ is —NHCH₃. In some embodiments, X⁴ is —NH(C(═O)CH₃). In some embodiments, X⁴ is —CH₃. In some embodiments, X⁴ is —CH₂CH₃. In some embodiments, X⁴ is —CH₂CH₂CH₃. In some embodiments, X⁴ is —CH(CH₃)₂. In some embodiments, X⁴ is —OCH₃. In some embodiments, X⁴ is —OCH₂CH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Xⁿ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, Xⁿ is —OH. In some embodiments, Xⁿ is —SH. In some embodiments, Xⁿ is —O⁻. In some embodiments, Xⁿ is —S⁻. In some embodiments, Xⁿ is —NH₂. In some embodiments, Xⁿ is —NHCH₃. In some embodiments, Xⁿ is —NH(C(═O)CH₃). In some embodiments, Xⁿ is —CH₃. In some embodiments, Xⁿ is —CH₂CH₃. In some embodiments, Xⁿ is —CH₂CH₂CH₃. In some embodiments, Xⁿ is —CH(CH₃)₂. In some embodiments, Xⁿ is —OCH₃. In some embodiments, Xⁿ is —OCH₂CH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Y¹ is ═O, ═S, ═NH, or ═NCH₃. In some embodiments, Y¹ is ═O. In some embodiments, Y¹ is ═S. In some embodiments, Y¹ is ═NH. In some embodiments, Y¹ is ═NCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Y² is ═O, ═S, ═NH, or ═NCH₃. In some embodiments, Y² is ═O. In some embodiments, Y² is ═S. In some embodiments, Y² is ═NH. In some embodiments, Y² is ═NCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Y³ is ═O, ═S, ═NH, or ═NCH₃. In some embodiments, Y³ is ═O. In some embodiments, Y³ is ═S. In some embodiments, Y³ is ═NH. In some embodiments, Y³ is ═NCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Y⁴ is ═O, ═S, ═NH, or ═NCH₃. In some embodiments, Y⁴ is ═O. In some embodiments, Y⁴ is ═S. In some embodiments, Y⁴ is ═NH. In some embodiments, Y⁴ is ═NCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), Yⁿ is ═O, ═S, ═NH, or ═NCH₃. In some embodiments, Yⁿ is ═O. In some embodiments, Yⁿ is ═S. In some embodiments, Yⁿ is ═NH. In some embodiments, Yⁿ is ═NCH₃.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), A is —O—, —S—, —CH₂—, —NH—, —N(CH₃)— or —N(C(═O)CH₃)—. In some embodiments, A is —O—. In some embodiments, A is —S—. In some embodiments, A is —CH₂—. In some embodiments, A is —NH—. In some embodiments, A is —N(CH₃)—. In some embodiments, A is —N(C(═O)CH₃)—.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), A¹ is —O—, —S—, —CH₂—, —NH—, —N(CH₃)— or —N(C(═O)CH₃)—. In some embodiments, A¹ is —O—. In some embodiments, A¹ is —S—. In some embodiments, A¹ is —CH₂—. In some embodiments, A¹ is —NH—. In some embodiments, A¹ is —N(CH₃)—. In some embodiments, A¹ is —N(C(═O)CH₃)—.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), A² is —O—, —S—, —CH₂—, —NH—, —N(CH₃)— or —N(C(═O)CH₃)—. In some embodiments, A² is —O—. In some embodiments, A² is —S—. In some embodiments, A² is —CH₂—. In some embodiments, A² is —NH—. In some embodiments, A² is —N(CH₃)—. In some embodiments, A² is —N(C(═O)CH₃)—.

In some embodiments of a compound of Formula (II), (II-a), (II-b), (II-c), (II-d), (II-e), (II-f), (II-g), (II-h), (II-i), (II-j), (II-k), (II-l), (II-m), (II-n), (II-o), (II-p), (II-q), (II-r), (II-s), (II-t), or (II-u), p is 0, 1, 2, 3, 4, 5 or 6. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments, B¹ is

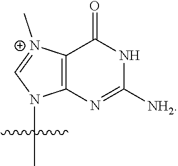 [SMILES: *n1c[n+](C)c2c(=O)[nH]c(N)nc21]

In some embodiments, B¹ is

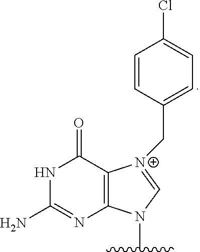 [SMILES: *n1c[n+](Cc2ccc(Cl)cc2)c2c(=O)[nH]c(N)nc21]

In some embodiments, each B² and B³ is independently adenine or guanine. In some embodiments, B² is adenine. In some embodiments, B³ is guanine.

In some embodiments, each Z¹, Z², Z³, and Z⁴ is independently —OH or —OCH₃. In some embodiments, Z³ is —OCH₃. In some embodiments, Z¹ is —OCH₃. In some embodiments Z² is —OH. In some embodiments, Z¹, Z², and Z⁴ are —OH and Z³ is —OCH₃. In some embodiments, Z¹ and Z³ are —OCH₃ and Z² and Z⁴ are —OH. In some embodiments, Z′, Z″, and Z′″ are hydrogen.

In some embodiments, Q¹ and Q⁴ are —OCH₂—. In some embodiments Q² and Q³ are —O—.

In some embodiments, X¹, X², X³, and X⁴ are —O—. In some embodiments, X¹, X², X³, and X⁴ are —S—. In some embodiments, X¹, X³, X⁴ are —O— and X² is —S—.

In some embodiments, Y¹, Y², Y³, and Y⁴ are ═O. In some embodiments, Y¹, Y², Y³, and Y⁴ are ═S. In some embodiments, Y¹, Y³, and Y⁴ are ═O and Y² is ═S. In some embodiments, Y¹, Y², Y³ are ═O and Y⁴ is ═S.

In some embodiments, A, A¹, and A² are —O—.

In some embodiments, p is 0.

In one aspect, described herein is an mRNA sequence having a 5′-end region motif (Motif (II′)

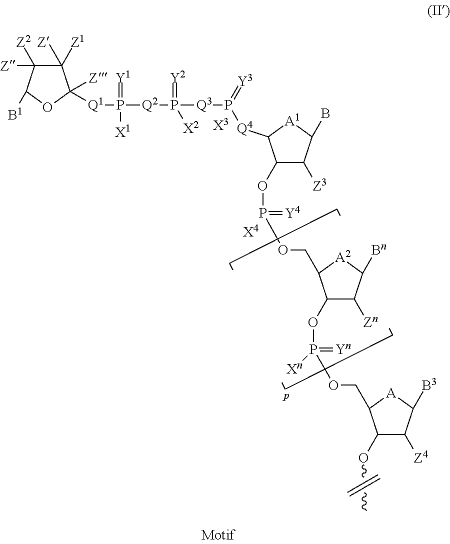

wherein
B¹ is

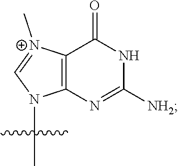 [SMILES: *n1c[n+](C)c2c(=O)[nH]c(N)nc21]

each B², B³, and Bⁿ is independently a natural, a modified, or an unnatural nucleobase;
each Z¹ and Z′ is independently is hydrogen, fluorine, —OH, —SH, —CH₃, —CH₂CH₃, —OCH₃, —NH(CH₃), —NH₂, —NH(C(═O)CH₃), or —SCH₃;
each Z² and Z″ is independently fluorine, —OH, —SH, —CH₃, —CH₂CH₃, —OCH₃, —SCH₃, —OCH₂CH₃, —NH₂, NHCH₃, or NHC(═O)CH₃;
Z′″ is hydrogen, fluorine, —CH₃, —CH₂CH₃, —OCH₃, or —OCH₂CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), B² independently a natural, a modified, or an unnatural nucleobase. In some embodiments, B² is adenine. In some embodiments, B² is guanine. In some embodiments, B2 is cytosine. In some embodiments, B² is uracil, In some embodiments, B² is thymine, In some embodiments, B² is hypoxanthine. In some embodiments, B² is purine.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), B³ independently a natural, a modified, or an unnatural nucleobase. In some embodiments, B³ is adenine. In some embodiments, B³ is guanine. In some embodiments, B3 is cytosine. In some embodiments, B³ is uracil, In some embodiments, B³ is thymine, In some embodiments, B³ is hypoxanthine. In some embodiments, B³ is purine.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Bⁿ independently a natural, a modified, or an unnatural nucleobase. In some embodiments, Bⁿ is adenine. In some embodiments, Bⁿ is guanine. In some embodiments, Bⁿ is cytosine. In some embodiments, Bⁿ is uracil, In some embodiments, Bⁿ is thymine, In some embodiments, Bⁿ is hypoxanthine. In some embodiments, Bⁿ is purine.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Z¹ is hydrogen, fluorine, —OH, —SH, —CH₃, —CH₂CH₃, —OCH₃, —NH(CH₃), —NH₂, —NH(C(═O)CH₃), or —SCH₃. In some embodiments, Z¹ is hydrogen. In some embodiments, Z¹ is fluorine. In some embodiments, Z¹ is —OH. In some embodiments, Z¹ is —SH. In some embodiments, Z¹ is —CH₃. In some embodiments, Z¹ is —CH₂CH₃. In some embodiments, Z¹ is —OCH₃. In some embodiments, Z¹ is —NH(CH₃), In some embodiments, Z¹ is —NH₂—. In some embodiments, Z¹ is —NH(C(═O)CH₃). In some embodiments, Z¹ is —SCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Z′ is hydrogen, fluorine, —OH, —SH, —CH₃, —CH₂CH₃, —OCH₃, —NH(CH₃), —NH₂, —NH(C(═O)CH₃), or —SCH₃. In some embodiments, Z′ is hydrogen. In some embodiments, Z′ is fluorine. In some embodiments, Z′ is —OH. In some embodiments, Z′ is —SH. In some embodiments, Z′ is —CH₃. In some embodiments, Z′ is —CH₂CH₃. In some embodiments, Z′ is —OCH₃. In some embodiments, Z′ is —NH(CH₃), In some embodiments, Z′ is —NH₂—. In some embodiments, Z′ is —NH(C(═O)CH₃). In some embodiments, Z′ is —SCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Z² is fluorine, —OH, —SH, —CH₃, —CH₂CH₃, —OCH₃, —SCH₃, —OCH₂CH₃, —NH₂, NHCH₃, or NHC(═O)CH₃. In some embodiments, Z² is fluorine. In some embodiments, Z² is —OH. In some embodiments, Z² is —SH. In some embodiments, Z² is —CH₃. In some embodiments, Z² is —CH₂CH₃. In some embodiments, Z² is —OCH₃. In some embodiments, Z² is —SCH₃. In some embodiments, Z² is —OCH₂CH₃. In some embodiments, Z² is —NH2. In some embodiments, Z² is NHCH₃. In some embodiments, Z² is NHC(═O)CH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Zⁿ is fluorine, —OH, —SH, —CH₃, —CH₂CH₃, —OCH₃, —SCH₃, —OCH₂CH₃, —NH₂, NHCH₃, or NHC(═O)CH₃. In some embodiments, Z″ is fluorine. In some embodiments, Z″ is —OH. In some embodiments, Z″ is —SH. In some embodiments, Z″ is —CH₃. In some embodiments, Z″ is —CH₂CH₃. In some embodiments, Z″ is —OCH₃. In some embodiments, Z″ is —SCH₃. In some embodiments, Z″ is —OCH₂CH₃. In some embodiments, Z″ is —NH₂. In some embodiments, Z″ is NHCH₃. In some embodiments, Z″ is NHC(═O)CH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Z′″ is hydrogen, fluorine, —CH₃, —CH₂CH₃, —OCH₃, or —OCH₂CH₃. In some embodiments, Z′″ is hydrogen. In some embodiments, Z′″ is fluorine. In some embodiments, Z′″ is —CH₃. In some embodiments, Z′″ is —CH₂CH₃. In some embodiments, Z′″ is —OCH₃. In some embodiments, Z′″ is —OCH₂CH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Z³ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z³ is hydrogen. In some embodiments, Z³ is fluorine. In some embodiments, Z³ is —OH. In some embodiments, Z³ is —CH₃. In some embodiments, Z³ is —CH₂CH₃. In some embodiments, Z³ is —OCH₃. In some embodiments, Z³ is —NH₂. In some embodiments, Z³ is —NHCH₃. In some embodiments, Z³ is —NH(C(═O)CH₃). In some embodiments, Z³ is —OCH₂CH₃. In some embodiments, Z³ is —OCH₂OCH₃. In some embodiments, Z³ is —OCH₂CH₂CH₃. In some embodiments, Z³ is —OCH(CH₃)₂. In some embodiments, Z³ is —SCH₃. In some embodiments, Z³ is —OCH₂CH₂OCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Z⁴ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z⁴ is hydrogen. In some embodiments, Z⁴ is fluorine. In some embodiments, Z⁴ is —OH. In some embodiments, Z⁴ is —CH₃. In some embodiments, Z⁴ is —CH₂CH₃. In some embodiments, Z⁴ is —OCH₃. In some embodiments, Z⁴ is —NH₂. In some embodiments, Z⁴ is —NHCH₃. In some embodiments, Z⁴ is —NH(C(═O)CH₃). In some embodiments, Z⁴ is —OCH₂CH₃. In some embodiments, Z⁴ is —OCH₂OCH₃. In some embodiments, Z⁴ is —OCH₂CH₂CH₃. In some embodiments, Z⁴ is —OCH(CH₃)₂. In some embodiments, Z⁴ is —SCH₃. In some embodiments, Z⁴ is —OCH₂CH₂OCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Zⁿ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Zⁿ is hydrogen. In some embodiments, Zⁿ is fluorine. In some embodiments, Zⁿ is —OH. In some embodiments, Zⁿ is —CH₃. In some embodiments, Zⁿ is —CH₂CH₃. In some embodiments, Zⁿ is —OCH₃. In some embodiments, Zⁿ is —NH₂. In some embodiments, Zⁿ is —NHCH₃. In some embodiments, Zⁿ is —NH(C(═O)CH₃). In some embodiments, Zⁿ is —OCH₂CH₃. In some embodiments, Zⁿ is —OCH₂OCH₃. In some embodiments, Zⁿ is —OCH₂CH₂CH₃. In some embodiments, Zⁿ is —OCH(CH₃)₂. In some embodiments, Zⁿ is —SCH₃. In some embodiments, Zⁿ is —OCH₂CH₂OCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Q¹ is —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH═CH—. In some embodiments, Q¹ is —CH₂—. In some embodiments, Q¹ is —CH₂O—. In some embodiments, Q¹ is —CH₂S—. In some embodiments, Q¹ is —CH₂CH₂—. In some embodiments, Q¹ is —CH₂CF₂—. In some embodiments, Q¹ is —CH₂NH₂—. In some embodiments, Q¹ is —CH₂NH(CH₃)—. In some embodiments, Q¹ is —CH₂N(C(═O)CH₃)—.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Q⁴ is —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH═CH—. In some embodiments, Q⁴ is —CH₂—. In some embodiments, Q⁴ is —CH₂O—. In some embodiments, Q⁴ is —CH₂S—. In some embodiments, Q⁴ is —CH₂CH₂—. In some embodiments, Q⁴ is —CH₂CF₂—. In some embodiments, Q⁴ is —CH₂NH₂—. In some embodiments, Q⁴ is —CH₂NH(CH₃)—. In some embodiments, Q⁴ is —CH₂N(C(═O)CH₃)—.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Q² is —O—, —S—, —CH₂—, —CF₂—, —NH—, —N(CH₃)—, or —N(C(═O)CH₃)—. In some embodiments Q² is —O—. In some embodiments Q² is —S—. In some embodiments Q² is —CH₂—. In some embodiments Q² is —CF₂—. In some embodiments Q² is —NH—. In some embodiments Q² is —N(CH₃)—. In some embodiments Q² is —N(C(═O)CH₃)—.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Q³ is —O—, —S—, —CH₂—, —CF₂—, —NH—, —N(CH₃)—, or —N(C(═O)CH₃)—. In some embodiments Q³ is —O—. In some embodiments Q³ is —S—. In some embodiments Q³ is —CH₂—. In some embodiments Q³ is —CF₂—. In some embodiments Q³ is —NH—. In some embodiments Q³ is —N(CH₃)—. In some embodiments Q³ is —N(C(═O)CH₃)—.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), X¹ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X¹ is —OH. In some embodiments, X¹ is —SH. In some embodiments, X¹ is —O⁻. In some embodiments, X¹ is —S⁻. In some embodiments, X¹ is —NH₂. In some embodiments, X¹ is —NHCH₃. In some embodiments, X¹ is —NH(C(═O)CH₃). In some embodiments, X¹ is —CH₃. In some embodiments, X¹ is —CH₂CH₃. In some embodiments, X¹ is —CH₂CH₂CH₃. In some embodiments, X¹ is —CH(CH₃)₂. In some embodiments, X¹ is —OCH₃. In some embodiments, X¹ is —OCH₂CH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), X² is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X² is —OH. In some embodiments, X² is —SH. In some embodiments, X² is —O⁻. In some embodiments, X² is —S⁻. In some embodiments, X² is —NH₂. In some embodiments, X² is —NHCH₃. In some embodiments, X² is —NH(C(═O)CH₃). In some embodiments, X² is —CH₃. In some embodiments, X² is —CH₂CH₃. In some embodiments, X² is —CH₂CH₂CH₃. In some embodiments, X² is —CH(CH₃)₂. In some embodiments, X² is —OCH₃. In some embodiments, X² is —OCH₂CH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), X³ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X³ is —OH. In some embodiments, X³ is —SH. In some embodiments, X³ is —O⁻. In some embodiments, X³ is —S⁻. In some embodiments, X³ is —NH₂. In some embodiments, X³ is —NHCH₃. In some embodiments, X³ is —NH(C(═O)CH₃). In some embodiments, X³ is —CH₃. In some embodiments, X³ is —CH₂CH₃. In some embodiments, X³ is —CH₂CH₂CH₃. In some embodiments, X³ is —CH(CH₃)₂. In some embodiments, X³ is —OCH₃. In some embodiments, X³ is —OCH₂CH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), X⁴ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X⁴ is —OH. In some embodiments, X⁴ is —SH. In some embodiments, X⁴ is —O⁻. In some embodiments, X⁴ is —S⁻. In some embodiments, X⁴ is —NH₂. In some embodiments, X⁴ is —NHCH₃. In some embodiments, X⁴ is —NH(C(═O)CH₃). In some embodiments, X⁴ is —CH₃. In some embodiments, X⁴ is —CH₂CH₃. In some embodiments, X⁴ is —CH₂CH₂CH₃. In some embodiments, X⁴ is —CH(CH₃)₂. In some embodiments, X⁴ is —OCH₃. In some embodiments, X⁴ is —OCH₂CH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Xⁿ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, Xⁿ is —OH. In some embodiments, Xⁿ is —SH. In some embodiments, Xⁿ is —O⁻. In some embodiments, Xⁿ is —S⁻. In some embodiments, Xⁿ is —NH₂. In some embodiments, Xⁿ is —NHCH₃. In some embodiments, Xⁿ is —NH(C(═O)CH₃). In some embodiments, Xⁿ is —CH₃. In some embodiments, Xⁿ is —CH₂CH₃. In some embodiments, Xⁿ is —CH₂CH₂CH₃. In some embodiments, Xⁿ is —CH(CH₃)₂. In some embodiments, Xⁿ is —OCH₃. In some embodiments, Xⁿ is —OCH₂CH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Y¹ is ═O, ═S, ═NH, or ═NCH₃. In some embodiments, Y¹ is ═O. In some embodiments, Y¹ is ═S. In some embodiments, Y¹ is ═NH. In some embodiments, Y¹ is ═NCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Y² is ═O, ═S, ═NH, or ═NCH₃. In some embodiments, Y² is ═O. In some embodiments, Y² is ═S. In some embodiments, Y² is ═NH. In some embodiments, Y² is ═NCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Y³ is ═O, ═S, ═NH, or ═NCH₃. In some embodiments, Y³ is ═O. In some embodiments, Y³ is ═S. In some embodiments, Y³ is ═NH. In some embodiments, Y³ is ═NCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Y⁴ is ═O, ═S, ═NH, or ═NCH₃. In some embodiments, Y⁴ is ═O. In some embodiments, Y⁴ is ═S. In some embodiments, Y⁴ is ═NH. In some embodiments, Y⁴ is ═NCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), Yⁿ is ═O, ═S, ═NH, or ═NCH₃. In some embodiments, Yⁿ is ═O. In some embodiments, Yⁿ is ═S. In some embodiments, Yⁿ is ═NH. In some embodiments, Yⁿ is ═NCH₃.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), A is —O—, —S—, —CH₂—, —NH—, —N(CH₃)— or —N(C(═O)CH₃)—. In some embodiments, A is —O—. In some embodiments, A is —S—. In some embodiments, A is —CH₂—. In some embodiments, A is —NH—. In some embodiments, A is —N(CH₃)—. In some embodiments, A is —N(C(═O)CH₃)—.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), A¹ is —O—, —S—, —CH₂—, —NH—, —N(CH₃)— or —N(C(═O)CH₃)—. In some embodiments, A¹ is —O—. In some embodiments, A¹ is —S—. In some embodiments, A¹ is —CH₂—. In some embodiments, A¹ is —NH—. In some embodiments, A¹ is —N(CH₃)—. In some embodiments, A¹ is —N(C(═O)CH₃)—.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), A² is —O—, —S—, —CH₂—, —NH—, —N(CH₃)— or —N(C(═O)CH₃)—. In some embodiments, A² is —O—. In some embodiments, A² is —S—. In some embodiments, A² is —CH₂—. In some embodiments, A² is —NH—. In some embodiments, A² is —N(CH₃)—. In some embodiments, A² is —N(C(═O)CH₃)—.

In some embodiments of a compound of Motif (II′), (II′-a), (II′-b), (II′-c), (II′-d), (II′-e), (II′-f), (II′-g), (II′-h), (II′-i), (II′-j), (II′-k), (II′-l), (II′-m), (II′-n), (II′-o), (II′-p), (II′-q), (II′-r), (II′-s), (II′-t), or (II′-u), p is 0, 1, 2, 3, 4, 5 or 6. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments, B¹ is

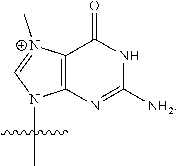 [SMILES: *n1c[n+](C)c2c(=O)[nH]c(N)nc21]

In some embodiments, each B² and B³ is independently adenine or guanine. In some embodiments, B² is adenine. In some embodiments, B³ is guanine.

In some embodiments, each Z¹, Z^{2′} Z³, and Z⁴ is independently —OH or —OCH₃. In some embodiments, Z³ is —OCH₃. In some embodiments, Z¹ is —OCH₃. In some embodiments Z² is —OH. In some embodiments, Z¹, Z², and Z⁴ are —OH and Z³ is —OCH₃. In some embodiments, Z¹ and Z³ are —OCH₃ and Z² and Z⁴ are —OH. In some embodiments, Z′, Z″, and Z′″ are hydrogen.

In some embodiments, Q¹ and Q⁴ are —OCH₂—. In some embodiments Q² and Q³ are —O—.

In some embodiments, X¹, X², X³, and X⁴ are —O—. In some embodiments, X¹, X², X³, and X⁴ are —S—. In some embodiments, X¹, X³, X⁴ are —O— and X² is —S—.

In some embodiments, Y¹, Y², Y³, and Y⁴ are ═O. In some embodiments, Y¹, Y², Y³, and Y⁴ are ═S. In some embodiments, Y¹, Y³, and Y⁴ are ═O and Y² is ═S. In some embodiments, Y¹, Y², Y³ are ═O and Y⁴ is ═S.

In some embodiments, A, A¹, and A² are —O—.

In some embodiments, p is 0.

In one aspect, described herein is an mRNA sequence initiator comprising a compound of Formula (II) or a salt or solvate thereof:

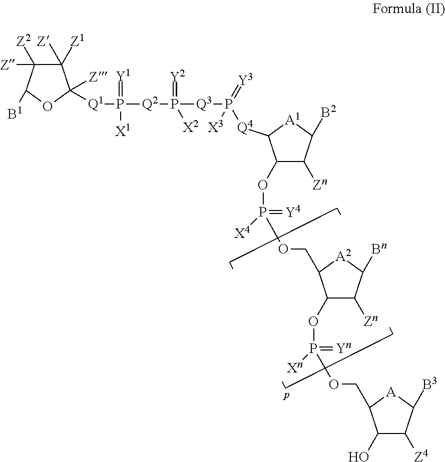

wherein
B¹ is

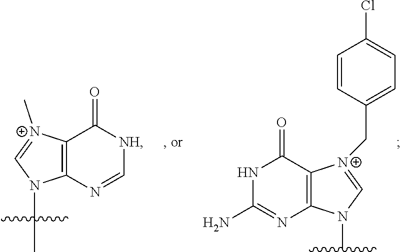 [SMILES: *n1c[n+](C)c2c(=O)[nH]cnc21 ; ", or" ; *n1c[n+](Cc2ccc(Cl)cc2)c2c(=O)[nH]c(N)nc21 ; ";"]

each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

In some embodiments of a compound of Formula (II), (II-a′), (II-b′), (II-c′), (II-d′), (II-e′), (II-f′) (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), B¹ is

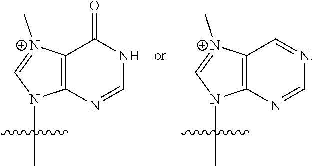 [SMILES: *n1c[n+](C)c2c(=O)[nH]cnc21 ; "or" ; *n1c[n+](C)c2cncnc21]

In some embodiments, B¹ is

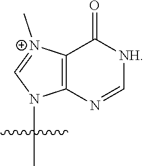 [SMILES: *n1c[n+](C)c2c(=O)[nH]cnc21]

In some embodiments, B¹ is

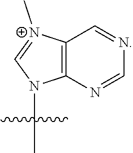 [SMILES: *n1c[n+](C)c2cncnc21]

In some embodiments, B¹ is

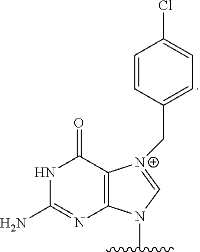 [SMILES: *n1c[n+](Cc2ccc(Cl)cc2)c2c(=O)[nH]c(N)nc21]

In some embodiments of a compound of Formula (II), (II-a′), (II-b′), (II-c′), (II-d′), (II-e′), (II-f′), (II-g′), (II-h′), (II-i′), (II-j′), (II-k′), (II-m′), (II-n′), (II-o′), (II-p′), (II-q′), (II-r′), (II-s′), (II-t′), or (II-u′), B² independently a natural, a modified, or an unnatural nucleobase. In some embodiments, B² is adenine. In some embodiments, B² is guanine. In some embodiments, B2 is cytosine. In some embodiments, B² is uracil, In some embodiments, B² is thymine, In some embodiments, B² is hypoxanthine. In some embodiments, B² is purine.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), B³ independently a natural, a modified, or an unnatural nucleobase. In some embodiments, B³ is adenine. In some embodiments, B³ is guanine. In some embodiments, B³ is cytosine. In some embodiments, B³ is uracil, In some embodiments, B³ is thymine, In some embodiments, B³ is hypoxanthine. In some embodiments, B³ is purine.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Bⁿ independently a natural, a modified, or an unnatural nucleobase. In some embodiments, Bⁿ is adenine. In some embodiments, Bⁿ is guanine. In some embodiments, Bⁿ is cytosine. In some embodiments, Bⁿ is uracil, In some embodiments, Bⁿ is thymine, In some embodiments, Bⁿ is hypoxanthine. In some embodiments, Bⁿ is purine.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Z′ is hydrogen, fluorine, —OH, —SH, —CH₃, —CH₂CH₃, —OCH₃, —NH(CH₃), —NH₂, —NH(C(═O)CH₃), or —SCH₃. In some embodiments, Z′ is hydrogen. In some embodiments, Z′ is fluorine. In some embodiments, Z′ is —OH. In some embodiments, Z′ is —SH. In some embodiments, Z′ is —CH₃. In some embodiments, Z′ is —CH₂CH₃. In some embodiments, Z′ is —OCH₃. In some embodiments, Z′ is —NH(CH₃), In some embodiments, Z′ is —NH₂—. In some embodiments, Z′ is —NH(C(═O)CH₃). In some embodiments, Z′ is —SCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Z″ is hydrogen, fluorine, —OH, —SH, —CH₃, —CH₂CH₃, —OCH₃, —NH(CH₃), —NH₂, —NH(C(═O)CH₃), or —SCH₃. In some embodiments, Z″ is hydrogen. In some embodiments, Z″ is fluorine. In some embodiments, Z″ is —OH. In some embodiments, Z″ is —SH. In some embodiments, Z″ is —CH₃. In some embodiments, Z″ is —CH₂CH₃. In some embodiments, Z″ is —OCH₃. In some embodiments, Z″ is —NH(CH₃), In some embodiments, Z″ is —NH₂—. In some embodiments, Z″ is —NH(C(═O)CH₃). In some embodiments, Z″ is —SCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Z′″ is hydrogen, fluorine, —CH₃, —CH₂CH₃, —OCH₃, or —OCH₂CH₃. In some embodiments, Z′″ is hydrogen. In some embodiments, Z′″ is fluorine. In some embodiments, Z′″ is —CH₃. In some embodiments, Z′″ is —CH₂CH₃. In some embodiments, Z′″ is —OCH₃. In some embodiments, Z′″ is —OCH₂CH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Z¹ is hydrogen, fluorine, —OH, —SH, —CH₃, —CH₂CH₃, —OCH₃, —SCH₃, —OCH₂CH₃, —NH₂, NHCH₃, or NHC(═O)CH₃. In some embodiments, Z¹ is hydrogen. In some embodiments, Z¹ is fluorine. In some embodiments, Z¹ is —OH. In some embodiments, Z¹ is —SH. In some embodiments, Z¹ is —CH₃. In some embodiments, Z¹ is —CH₂CH₃. In some embodiments, Z¹ is —OCH₃. In some embodiments, Z¹ is —SCH₃. In some embodiments, Z¹ is —OCH₂CH₃. In some embodiments, Z¹ is —NH₂. In some embodiments, Z¹ is NHCH₃. In some embodiments, Z¹ is NHC(═O)CH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Z² is hydrogen, fluorine, —OH, —SH, —CH₃, —CH₂CH₃, —OCH₃, —SCH₃, —OCH₂CH₃, —NH₂, NHCH₃, or NHC(═O)CH₃. In some embodiments, Z² is hydrogen. In some embodiments, Z² is fluorine. In some embodiments, Z² is —OH. In some embodiments, Z² is —SH. In some embodiments, Z² is —CH₃. In some embodiments, Z² is —CH₂CH₃. In some embodiments, Z² is —OCH₃. In some embodiments, Z² is —SCH₃. In some embodiments, Z² is —OCH₂CH₃. In some embodiments, Z² is —NH₂. In some embodiments, Z² is NHCH₃. In some embodiments, Z² is NHC(═O)CH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Z³ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z³ is hydrogen. In some embodiments, Z³ is fluorine. In some embodiments, Z³ is —OH. In some embodiments, Z³ is —CH₃. In some embodiments, Z³ is —CH₂CH₃. In some embodiments, Z³ is —OCH₃. In some embodiments, Z³ is —NH₂. In some embodiments, Z³ is —NHCH₃. In some embodiments, Z³ is —NH(C(═O)CH₃). In some embodiments, Z³ is —OCH₂CH₃. In some embodiments, Z³ is —OCH₂OCH₃. In some embodiments, Z³ is —OCH₂CH₂CH₃. In some embodiments, Z³ is —OCH(CH₃)₂. In some embodiments, Z³ is —SCH₃. In some embodiments, Z³ is —OCH₂CH₂OCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Z⁴ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z⁴ is hydrogen. In some embodiments, Z⁴ is fluorine. In some embodiments, Z⁴ is —OH. In some embodiments, Z⁴ is —CH₃. In some embodiments, Z⁴ is —CH₂CH₃. In some embodiments, Z⁴ is —OCH₃. In some embodiments, Z⁴ is —NH₂. In some embodiments, Z⁴ is —NHCH₃. In some embodiments, Z⁴ is —NH(C(═O)CH₃). In some embodiments, Z⁴ is —OCH₂CH₃. In some embodiments, Z⁴ is —OCH₂OCH₃. In some embodiments, Z⁴ is —OCH₂CH₂CH₃. In some embodiments, Z⁴ is —OCH(CH₃)₂. In some embodiments, Z⁴ is —SCH₃. In some embodiments, Z⁴ is —OCH₂CH₂OCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Zⁿ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Zⁿ is hydrogen. In some embodiments, Zⁿ is fluorine. In some embodiments, Zⁿ is —OH. In some embodiments, Zⁿ is —CH₃. In some embodiments, Zⁿ is —CH₂CH₃. In some embodiments, Zⁿ is —OCH₃. In some embodiments, Zⁿ is —NH₂. In some embodiments, Zⁿ is —NHCH₃. In some embodiments, Zⁿ is —NH(C(═O)CH₃). In some embodiments, Zⁿ is —OCH₂CH₃. In some embodiments, Zⁿ is —OCH₂OCH₃. In some embodiments, Zⁿ is —OCH₂CH₂CH₃. In some embodiments, Zⁿ is —OCH(CH₃)₂. In some embodiments, Zⁿ is —SCH₃. In some embodiments, Zⁿ is —OCH₂CH₂OCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Q¹ is —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH═CH—. In some embodiments, Q¹ is —CH₂—. In some embodiments, Q¹ is —CH₂O—. In some embodiments, Q¹ is —CH₂S—. In some embodiments, Q¹ is —CH₂CH₂—. In some embodiments, Q¹ is —CH₂CF₂—. In some embodiments, Q¹ is —CH₂NH₂—. In some embodiments, Q¹ is —CH₂NH(CH₃)—. In some embodiments, Q¹ is —CH₂N(C(═O)CH₃)—.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Q⁴ is —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH═CH—. In some embodiments, Q⁴ is —CH₂—. In some embodiments, Q⁴ is —CH₂O—. In some embodiments, Q⁴ is —CH₂S—. In some embodiments, Q⁴ is —CH₂CH₂—. In some embodiments, Q⁴ is —CH₂CF₂—. In some embodiments, Q⁴ is —CH₂NH₂—. In some embodiments, Q⁴ is —CH₂NH(CH₃)—. In some embodiments, Q⁴ is —CH₂N(C(═O)CH₃)—.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Q² is —O—, —S—, —CH₂—, —CF₂—, —NH—, —N(CH₃)—, or —N(C(═O)CH₃)—. In some embodiments Q² is —O—. In some embodiments Q² is —S—. In some embodiments Q² is —CH₂—. In some embodiments Q² is —CF₂—. In some embodiments Q² is —NH—. In some embodiments Q² is —N(CH₃)—. In some embodiments Q² is —N(C(═O)CH₃)—.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Q³ is —O—, —S—, —CH₂—, —CF₂—, —NH—, —N(CH₃)—, or —N(C(═O)CH₃)—. In some embodiments Q³ is —O—. In some embodiments Q³ is —S—. In some embodiments Q³ is —CH₂—. In some embodiments Q³ is —CF₂—. In some embodiments Q³ is —NH—. In some embodiments Q³ is —N(CH₃)—. In some embodiments Q³ is —N(C(═O)CH₃)—.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), X¹ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X¹ is —OH. In some embodiments, X¹ is —SH. In some embodiments, X¹ is —O⁻. In some embodiments, X¹ is —S⁻. In some embodiments, X¹ is —NH₂. In some embodiments, X¹ is —NHCH₃. In some embodiments, X¹ is —NH(C(═O)CH₃). In some embodiments, X¹ is —CH₃. In some embodiments, X¹ is —CH₂CH₃. In some embodiments, X¹ is —CH₂CH₂CH₃. In some embodiments, X¹ is —CH(CH₃)₂. In some embodiments, X¹ is —OCH₃. In some embodiments, X¹ is —OCH₂CH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), X² is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X² is —OH. In some embodiments, X² is —SH. In some embodiments, X² is —O⁻. In some embodiments, X² is —S⁻. In some embodiments, X² is —NH₂. In some embodiments, X² is —NHCH₃. In some embodiments, X² is —NH(C(═O)CH₃). In some embodiments, X² is —CH₃. In some embodiments, X² is —CH₂CH₃. In some embodiments, X² is —CH₂CH₂CH₃. In some embodiments, X² is —CH(CH₃)₂. In some embodiments, X² is —OCH₃. In some embodiments, X² is —OCH₂CH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), X³ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X³ is —OH. In some embodiments, X³ is —SH. In some embodiments, X³ is —O⁻. In some embodiments, X³ is —S⁻. In some embodiments, X³ is —NH₂. In some embodiments, X³ is —NHCH₃. In some embodiments, X³ is —NH(C(═O)CH₃). In some embodiments, X³ is —CH₃. In some embodiments, X³ is —CH₂CH₃. In some embodiments, X³ is —CH₂CH₂CH₃. In some embodiments, X³ is —CH(CH₃)₂. In some embodiments, X³ is —OCH₃. In some embodiments, X³ is —OCH₂CH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), X⁴ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X⁴ is —OH. In some embodiments, X⁴ is —SH. In some embodiments, X⁴ is —O⁻. In some embodiments, X⁴ is —S⁻. In some embodiments, X⁴ is —NH₂. In some embodiments, X⁴ is —NHCH₃. In some embodiments, X⁴ is —NH(C(═O)CH₃). In some embodiments, X⁴ is —CH₃. In some embodiments, X⁴ is —CH₂CH₃. In some embodiments, X⁴ is —CH₂CH₂CH₃. In some embodiments, X⁴ is —CH(CH₃)₂. In some embodiments, X⁴ is —OCH₃. In some embodiments, X⁴ is —OCH₂CH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Xⁿ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, Xⁿ is —OH. In some embodiments, Xⁿ is —SH. In some embodiments, Xⁿ is —O⁻. In some embodiments, Xⁿ is —S⁻. In some embodiments, Xⁿ is —NH₂. In some embodiments, Xⁿ is —NHCH₃. In some embodiments, Xⁿ is —NH(C(═O)CH₃). In some embodiments, Xⁿ is —CH₃. In some embodiments, Xⁿ is —CH₂CH₃. In some embodiments, Xⁿ is —CH₂CH₂CH₃. In some embodiments, Xⁿ is —CH(CH₃)₂. In some embodiments, Xⁿ is —OCH₃. In some embodiments, Xⁿ is —OCH₂CH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Y¹ is ═O, ═S, ═NH, or ═NCH₃. In some embodiments, Y¹ is ═O. In some embodiments, Y¹ is ═S. In some embodiments, Y¹ is ═NH. In some embodiments, Y¹ is ═NCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Y² is ═O, ═S, ═NH, or ═NCH₃. In some embodiments, Y² is ═O. In some embodiments, Y² is ═S. In some embodiments, Y² is ═NH. In some embodiments, Y² is ═NCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Y³ is ═O, ═S, ═NH, or ═NCH₃. In some embodiments, Y³ is ═O. In some embodiments, Y³ is ═S. In some embodiments, Y³ is ═NH. In some embodiments, Y³ is ═NCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Y⁴ is ═O, ═S, ═NH, or ═NCH₃. In some embodiments, Y⁴ is ═O. In some embodiments, Y⁴ is ═S. In some embodiments, Y⁴ is ═NH. In some embodiments, Y⁴ is ═NCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), Yⁿ is ═O, ═S, ═NH, or ═NCH₃. In some embodiments, Yⁿ is ═O. In some embodiments, Yⁿ is ═S. In some embodiments, Yⁿ is ═NH. In some embodiments, Yⁿ is ═NCH₃.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), A is —O—, —S—, —CH₂—, —NH—, —N(CH₃)— or —N(C(═O)CH₃)—. In some embodiments, A is —O—. In some embodiments, A is —S—. In some embodiments, A is —CH₂—. In some embodiments, A is —NH—. In some embodiments, A is —N(CH₃)—. In some embodiments, A is —N(C(═O)CH₃)—.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), A¹ is —O—, —S—, —CH₂—, —NH—, —N(CH₃)— or —N(C(═O)CH₃)—. In some embodiments, A¹ is —O—. In some embodiments, A¹ is —S—. In some embodiments, A¹ is —CH₂—. In some embodiments, A¹ is —NH—. In some embodiments, A¹ is —N(CH₃)—. In some embodiments, A¹ is —N(C(═O)CH₃)—.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), A² is —O—, —S—, —CH₂—, —NH—, —N(CH₃)— or —N(C(═O)CH₃)—. In some embodiments, A² is —O—. In some embodiments, A² is —S—. In some embodiments, A² is —CH₂—. In some embodiments, A² is —NH—. In some embodiments, A² is —N(CH₃)—. In some embodiments, A² is —N(C(═O)CH₃)—.

In some embodiments of a compound of Formula (II), (II-a′), (II-b′), (II-c′), (II-d′), (II-e′), (II-f′), (II-g′), (II-h′), (II-i′), (II-j′), (II-k′), (II-l′), (II-m′), (II-n′), (II-o′), (II-p′), (II-q′), (II-r′), (II-s′), (II-t′), or (II-u′), p is 0, 1, 2, 3, 4, 5 or 6. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments, B¹ is

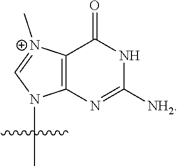 [SMILES: *n1c[n+](C)c2c(=O)[nH]c(N)nc21]

In some embodiments, each B² and B³ is independently adenine or guanine. In some embodiments, B² is adenine. In some embodiments, B³ is guanine.

In some embodiments, each Z¹, Z², Z³, and Z⁴ is independently —OH or —OCH₃. In some embodiments, Z³ is —OCH₃. In some embodiments, Z¹ is —OCH₃. In some embodiments Z² is —OH. In some embodiments, Z¹, Z², and Z⁴ are —OH and Z³ is —OCH₃. In some embodiments, Z¹ and Z³ are —OCH₃ and Z² and Z⁴ are —OH. In some embodiments, Z′, Z″, and Z′″ are hydrogen.

In some embodiments, Q¹ and Q⁴ are —OCH₂—. In some embodiments Q² and Q³ are —O—.

In some embodiments, X¹, X², X³, and X⁴ are —O—. In some embodiments, X¹, X², X³, and X⁴ are —S—. In some embodiments, X¹, X³, X⁴ are —O— and X² is —S—.

In some embodiments, Y¹, Y², Y³, and Y⁴ are ═O. In some embodiments, Y¹, Y², Y³, and Y⁴ are ═S. In some embodiments, Y¹, Y³, and Y⁴ are ═O and Y² is ═S. In some embodiments, Y¹, Y², Y³ are ═O and Y⁴ is ═S.

In some embodiments, A, A¹, and A² are —O—.

In some embodiments, p is 0.

In some embodiments, the IVT mRNA sequence initiator has a structure of Formula (II-a):

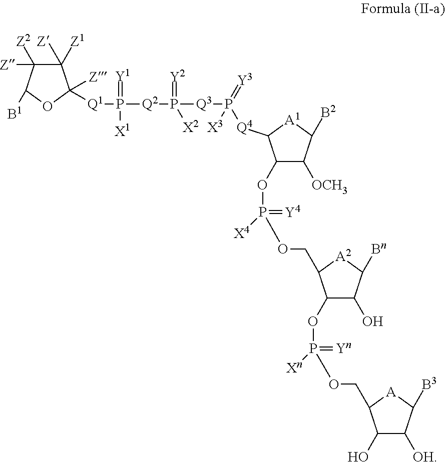

In some embodiments, the IVT mRNA sequence initiator has a structure of Formula (II-a′):

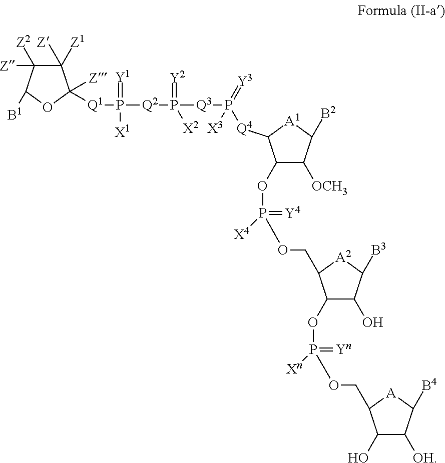

In some embodiments, the IVT mRNA sequence initiator has a structure of Formula (II-b):

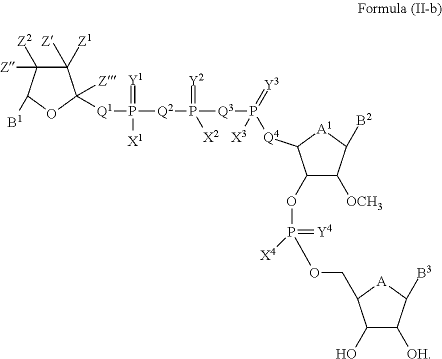

In some embodiments, the IVT mRNA sequence initiator has a structure of Formula (II-b′):

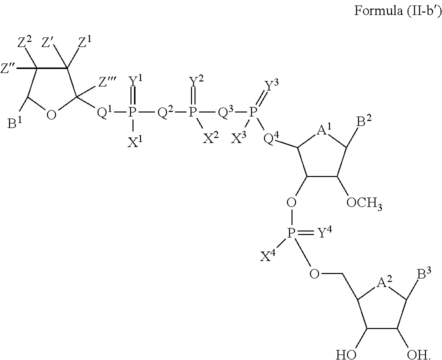

In some embodiments, the IVT mRNA sequence initiator has a structure of Formula (II-c):

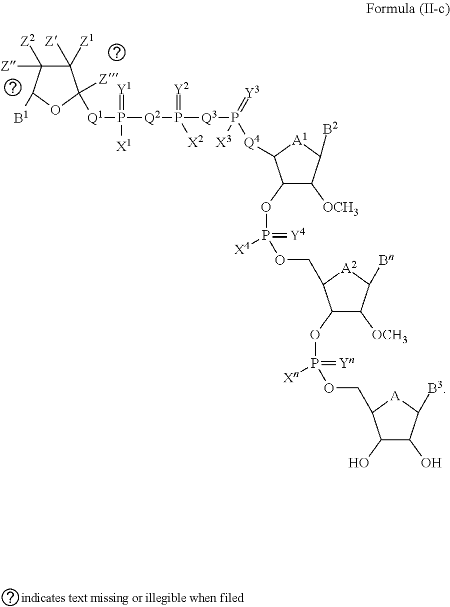

In some embodiments, the IVT mRNA sequence initiator has a structure of Formula (II-c′):

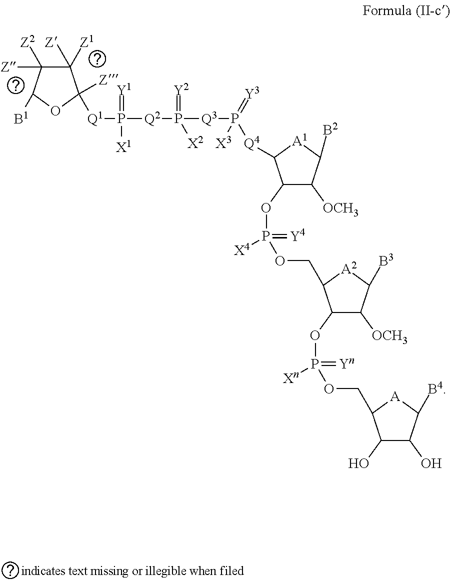

In some embodiments, the IVT mRNA sequence initiator has a structure of Formula (II-d):

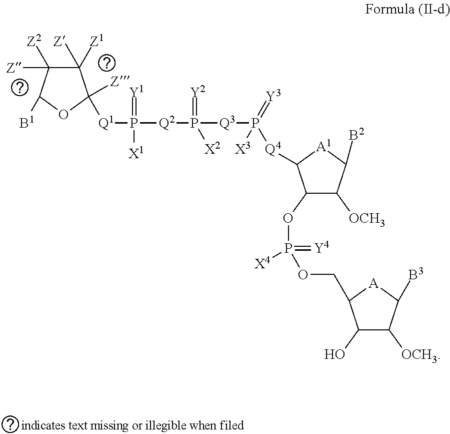

In some embodiments, the IVT mRNA sequence initiator has a structure of Formula (II-d′):

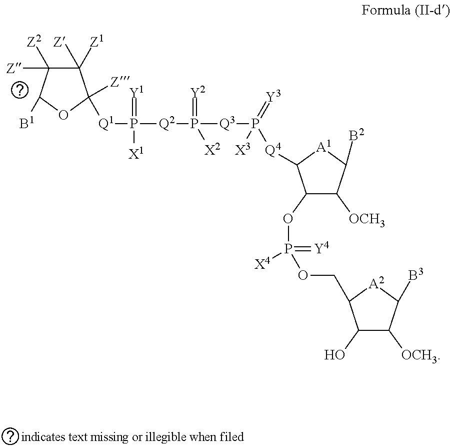

In some embodiments, the IVT mRNA sequence initiator has a structure of Formula (II-e):

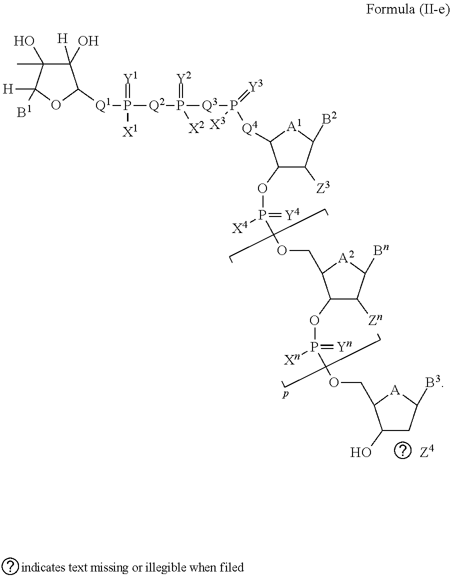

In some embodiments, the IVT mRNA sequence initiator has a structure of Formula (II-e′):

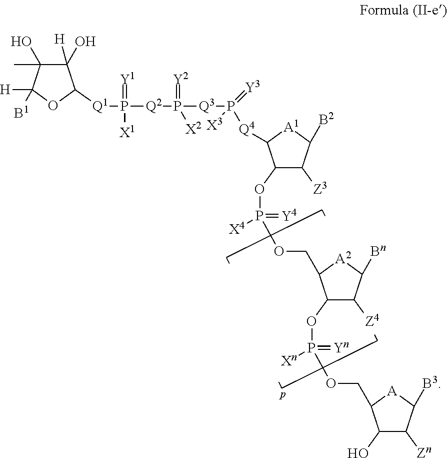

In some embodiments, the IVT mRNA sequence initiator has a structure of Formula (II-f):

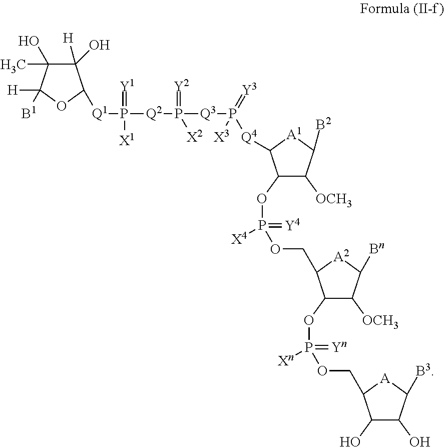

In some embodiments, the IVT mRNA sequence initiator has a structure of Formula (II-g):

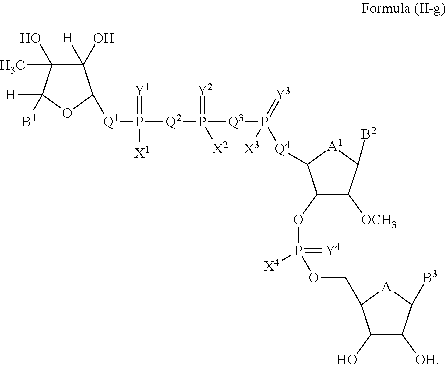

In some embodiments, the IVT mRNA sequence initiator has a structure of Formula (II-g′):

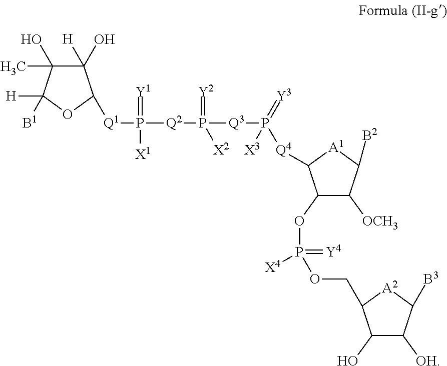

In some embodiments, the IVT mRNA sequence initiator has a structure of Formula (II-h):

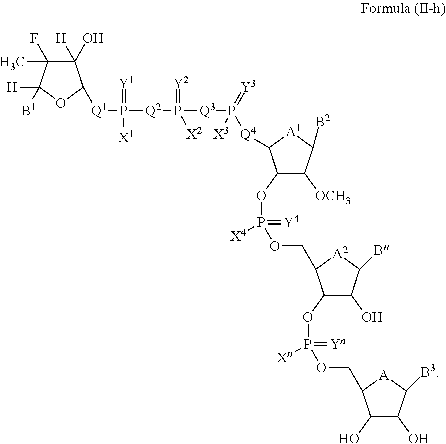

In some embodiments, the IVT mRNA sequence initiator has a structure of Formula (II-h′):

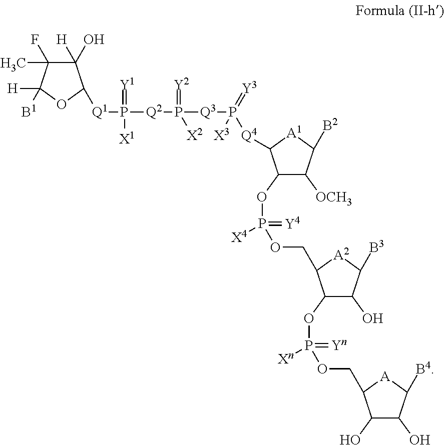

In some embodiments, the IVT mRNA sequence initiator has a structure of Formula (II-i)

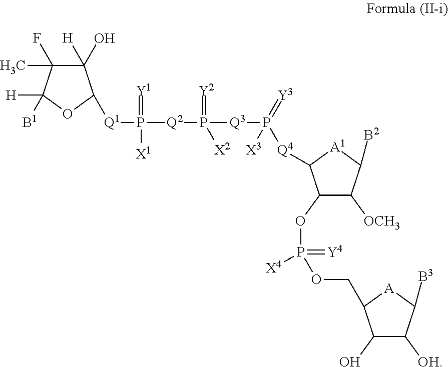

In some embodiments, the IVT mRNA sequence initiator has a structure of Formula (II-I′)

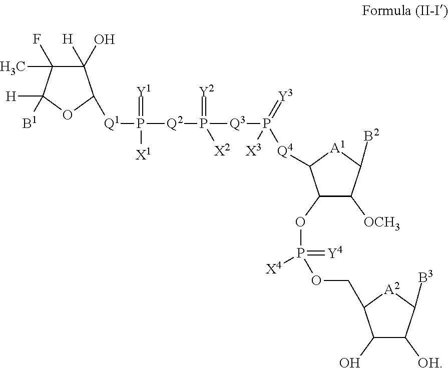

In some embodiments, the IVT mRNA sequence initiator has a structure of Formula (II-j):

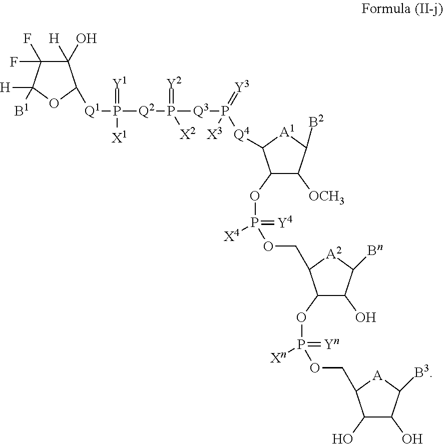

In some embodiments, the IVT mRNA sequence initiator has a structure of Formula (II-j′):

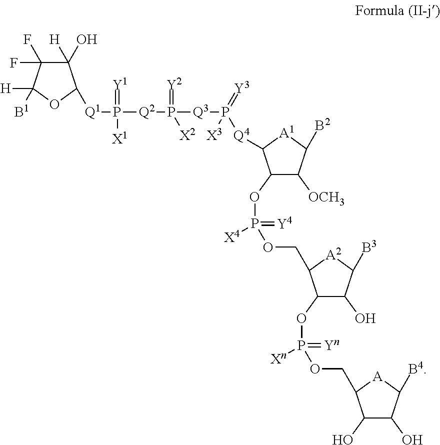

In some embodiments, the IVT mRNA sequence initiator has a structure of Formula (II-k):

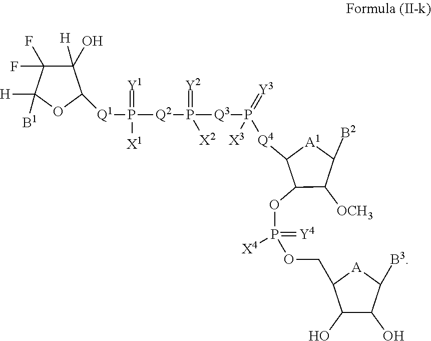

In some embodiments, the IVT mRNA sequence initiator has a structure of Formula (II-k′):

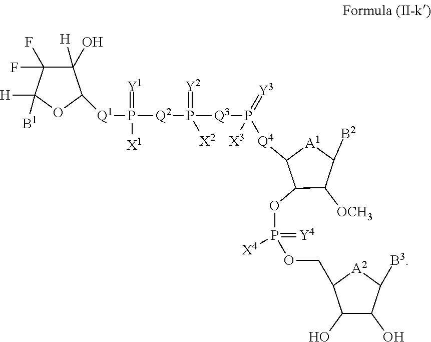

In some embodiments, the IVT mRNA sequence initiator has a structure of Formula (II-l):

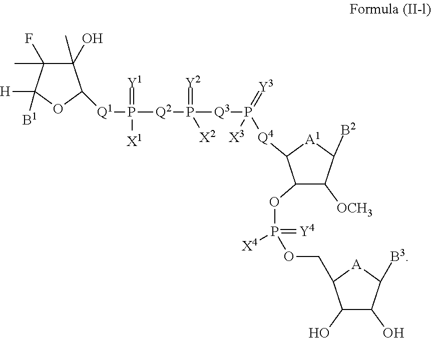

In some embodiments, the IVT mRNA sequence initiator has a structure of Formula (II-l′):

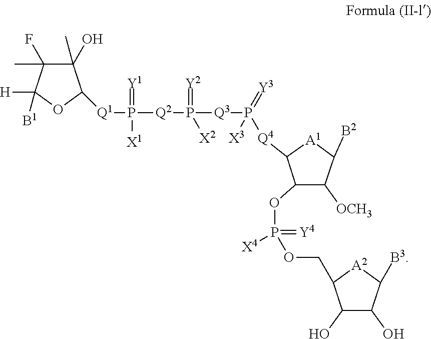

In some embodiments, the IVT mRNA sequence initiator has a structure of Formula (I-m):

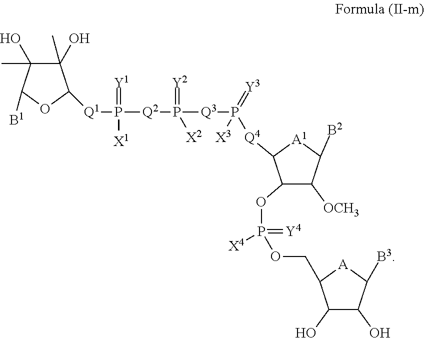

In some embodiments, the IVT mRNA sequence initiator has a structure of Formula (II-m′):

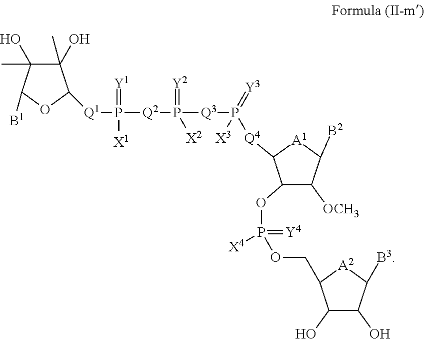

In some embodiments, the IVT mRNA sequence initiator has a structure of Formula (II-n):

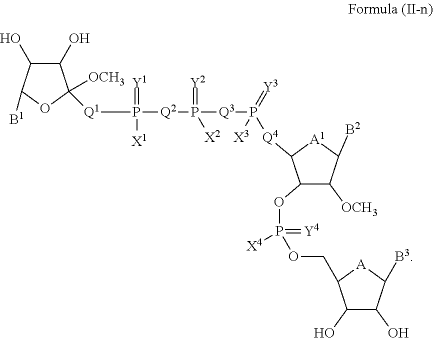

In some embodiments, the IVT mRNA sequence initiator has a structure of Formula (II-n′)

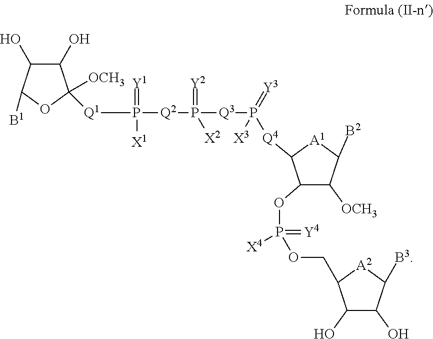

In some embodiments, the IVT mRNA sequence initiator has a structure of Formula (II-o):

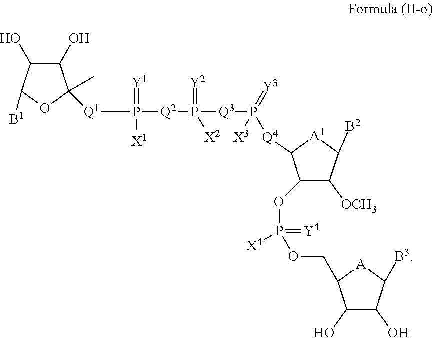

In some embodiments, the IVT mRNA sequence initiator has a structure of Formula (II-o′):

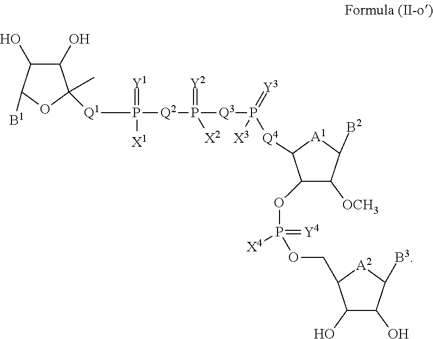

In some embodiments, the IVT mRNA sequence initiator has a structure of Formula (II-p):

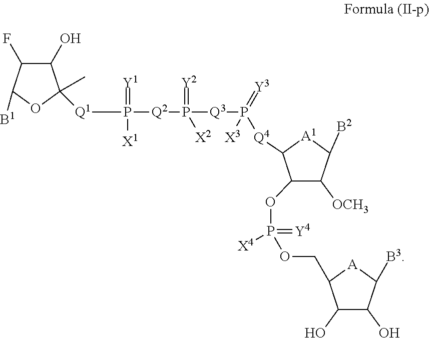

In some embodiments, the IVT mRNA sequence initiator has a structure of Formula (II-p′):

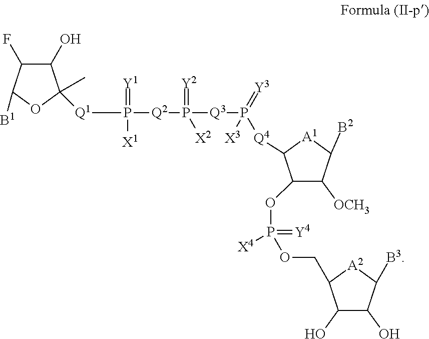

In some embodiments, the IVT mRNA sequence initiator has a structure of Formula (II-q):

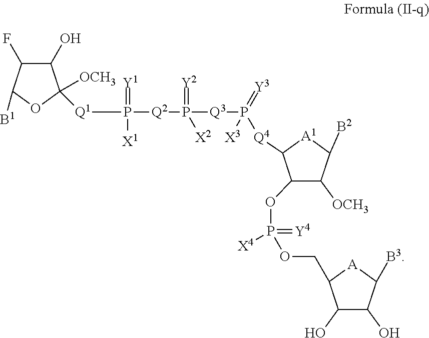

In some embodiments, the IVT mRNA sequence initiator has a structure of Formula (II-q′):

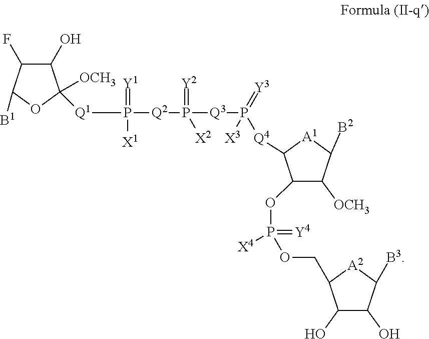

In some embodiments, the IVT mRNA sequence initiator has a structure of Formula (II-r):

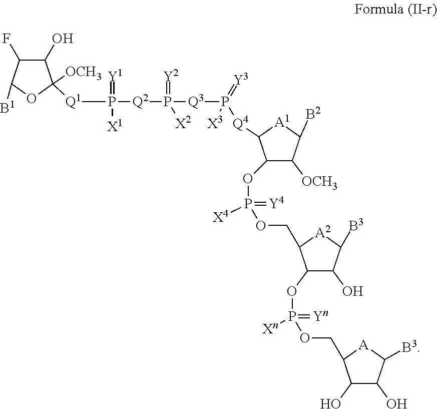

In some embodiments, the IVT mRNA sequence initiator has a structure of Formula (II-r′):

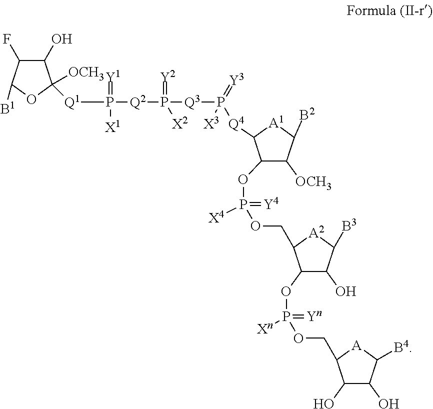

In some embodiments, the IVT mRNA sequence initiator has a structure of Formula (II-s):

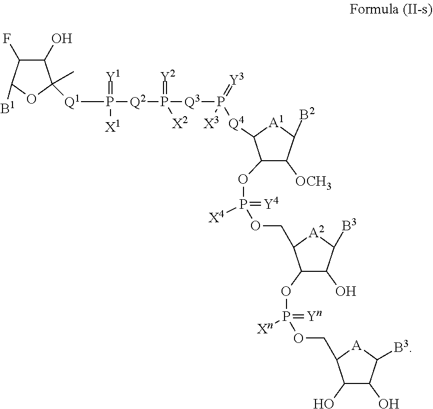

In some embodiments, the IVT mRNA sequence initiator has a structure of Formula (II-s′):

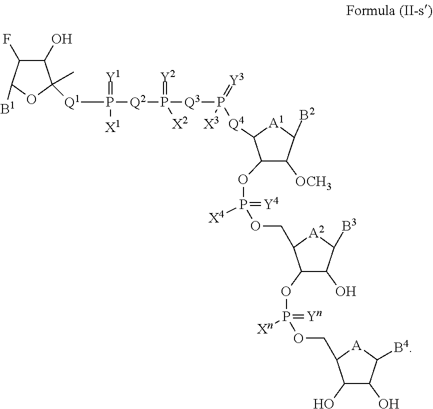

In some embodiments, the IVT mRNA sequence initiator has a structure of Formula (II-t):

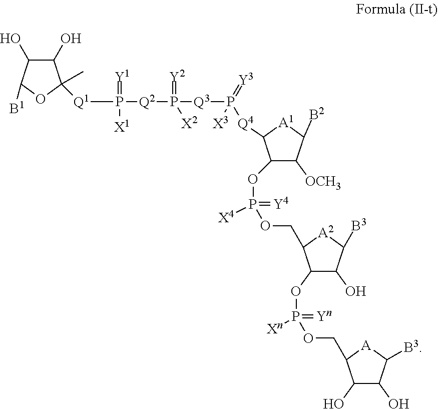

In some embodiments the IVT mRNA sequence initiator has a structure of Formula (II-t′):

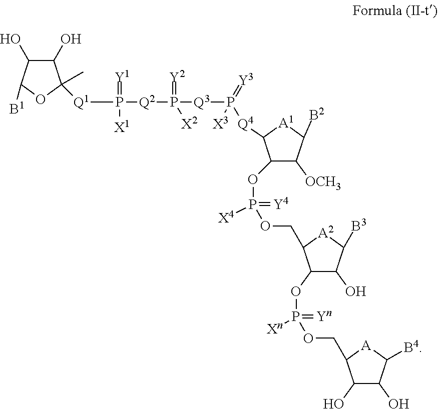

In some embodiments, the IVT mRNA sequence initiator has a structure of Formula (II-u):

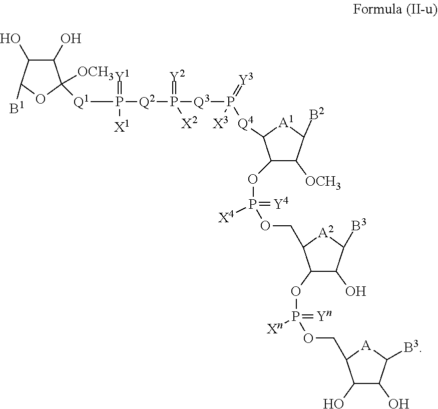

In some embodiments, the IVT mRNA sequence initiator has a structure of Formula (II-u′):

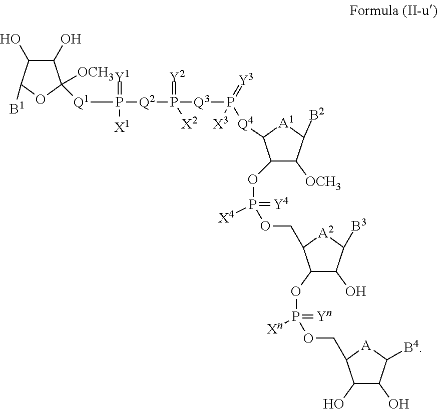

In some embodiments of Formula (II) or Formulas (II-a) to (II-u′), each B independently is a natural nucleobase. In some embodiments of Formula (II) or Formulas (II-a) to (II-u′), each B independently is a modified nucleobase. In some embodiments of Formula (II) or Formulas (II-a) to (II-u′), each B independently is an unnatural nucleobase.

In some embodiments of Formula (II) or Formulas (II-a) to (II-u′), B¹ is

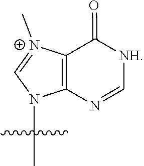 [SMILES: *n1c[n+](C)c2c(=O)[nH]cnc21]

In some embodiments of Formula (II) or Formulas (II-a) to (II-u′), B¹ is

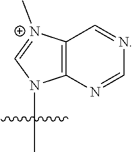 [SMILES: *n1c[n+](C)c2cncnc21]

In some embodiments of Formula (II) or Formulas (II-a) to (II-u′), B¹ is

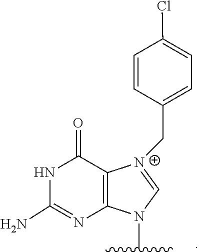 [SMILES: *n1c[n+](Cc2ccc(Cl)cc2)c2c(=O)[nH]c(N)nc21]

In some embodiments of Formula (II) or Formulas (II-a) to (II-u′), Z¹ is fluorine.

In some embodiments of Formula (II) or Formulas (II-a) to (II-u′), Z¹ is —OH.

In some embodiments of Formula (II) or Formulas (II-a) to (II-u′), Z¹ is —OCH₃.

In some embodiments of Formula (II) or Formulas (II-a) to (II-u′), Z² is fluorine.

In some embodiments of Formula (II) or Formulas (II-a) to (II-u′), Z² is —OH.

In some embodiments of Formula (II) or Formulas (II-a) to (II-u′), Z² is —OCH₃.

In some embodiments of Formula (II) or Formulas (II-a) to (II-u′), Q¹ is —CH₂O—.

In some embodiments of Formula (II) or Formulas (II-a) to (II-u′), Q¹ is —O—.

In some embodiments of Formula (II) or Formulas (II-a) to (II-u′), Q⁴ is —CH₂O—.

In some embodiments of Formula (II) or Formulas (II-a) to (II-u′), Q⁴ is —O—.

In some embodiments of Formula (II) or Formulas (II-a) to (II-u′), Q² is —O—.

In some embodiments of Formula (II) or Formulas (II-a) to (II-u′), Q³ is —O—.

In some embodiments of Formula (II) or Formulas (II-a) to (II-u′), Y¹ is ═O.

In some embodiments of Formula (II) or Formulas (II-a) to (II-u′), Y³ is ═O.

In some embodiments of Formula (II) or Formulas (II-a) to (II-u′), Y² is ═O.

In some embodiments of Formula (II) or Formulas (II-a) to (II-u′), Y⁴ is ═O.

In some embodiments of Formula (II) or Formulas (II-a) to (II-u′), Yⁿ is ═O.

In some embodiments of Formula (II) or Formulas (II-a) to (II-u′), one or more of Y¹, Y², Y³, Y⁴, and Yⁿ is ═S.

In some embodiments of Formula (II) or Formulas (II-a) to (II-u′), Y² is ═S.

In some embodiments of Formula (II) or Formulas (II-a) to (II-u′), Y⁴ is ═S.

In some embodiments of Formula (II) or Formulas (II-a) to (II-u′), Yⁿ is ═S.

In some embodiments of Formula (II) or Formulas (II-a) to (II-u′), each Y¹, Y², Y³, Y⁴, and Yⁿ is ═O.

In some embodiments of Formula (II) or Formulas (II-a) to (II-u′), each X¹, X⁴, and Xⁿ is —O—.

In some embodiments of Formula (II) or Formulas (II-a) to (II-u′), X² is —O—.

In some embodiments of Formula (II) or Formulas (II-a) to (II-u′), X³ is —O⁻.

In some embodiments of Formula (II) or Formulas (II-a) to (II-u′), one or more of X¹, X², X³, X⁴, and Xⁿ is —S⁻.

In some embodiments of Formula (II) or Formulas (II-a) to (II-u′), X² is —S⁻.

In some embodiments of Formula (II) or Formulas (II-a) to (II-u′), X⁴ is —S⁻.

In some embodiments of Formula (II) or Formulas (II-a) to (II-u′), X³ is —S—.

In some embodiments of Formula (II) or Formulas (II-a) to (II-u′), each X¹, X², X³, X⁴, and Xⁿ is —O⁻.

In some embodiments of Formula (II) or Formulas (II-a) to (II-u′), each A, A¹, and A² is —O—.

In some embodiments of Formula (II) or Formulas (II-a) to (II-u′), one or more of A, A¹, and A² is —S—. In some embodiments, A is —S—. In some embodiments, A¹ is —O—. In some embodiments, A² is —O—. In some embodiments, A² is —S—. In some embodiments, A is —O—. In some embodiments, A¹ is —O—.

In some embodiments of Formula (II) or Formulas (II-a) to (II-u′), p is 0. In some embodiments, p is 1. In some embodiments, p is 2.

In some embodiments of Formula (II) or Formulas (II-a) to (II-u′), B² is adenine, cytosine, guanine, uracil, thymine, hypoxanthine, or purine.

In some embodiments of Formula (II) or Formulas (II-a) to (II-u′), B³ is adenine, cytosine, guanine, uracil, thymine, hypoxanthine, or purine.

In some embodiments of Formula (II) or Formulas (II-a) to (II-u′), Bⁿ is adenine, cytosine, guanine, uracil, thymine, hypoxanthine, or purine.

In some embodiments of Formula (II) or Formulas (II-a) to (II-u′), Q¹ is —CH₂O—. In some embodiments, Q⁴ is —CH₂O—.

In some embodiments of Formula (II) or Formulas (II-a) to (II-u′), Q² is —O—. In some embodiments, Q³ is —O—.

In some embodiments of Formula (II) or Formulas (II-a) to (II-u′), each Xⁿ is independently —OH, —SH, O⁻, or S⁻.

In some embodiments of Formula (II) or Formulas (II-a) to (II-u′), each Yⁿ is independently ═O or ═S.

In some embodiments of Formula (II) or Formulas (II-a) to (II-u′), B¹ is

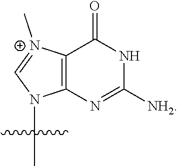 [SMILES: *n1c[n+](C)c2c(=O)[nH]c(N)nc21]

In some embodiments of Formula (II) or Formulas (II-a) to (II-u′), Q¹ is —CH₂O—; Q⁴ is —CH₂O—; Q² is —O—; Q³ is —O—; each Xⁿ is independently —OH, —SH, O⁻, or S⁻; each Yⁿ is independently ═O or ═S; B¹ is

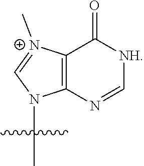 [SMILES: *n1c[n+](C)c2c(=O)[nH]cnc21]

In some embodiments of Formula (II) or Formulas (II-a) to (II-u′), Q¹ is —CH₂O—; Q⁴ is —CH₂O—; Q² is —O—; Q³ is —O—′ each Xⁿ is independently —OH, —SH, O⁻, or S⁻; each Yⁿ is independently ═O or ═S; B¹ is

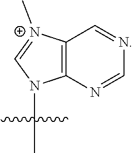 [SMILES: *n1c[n+](C)c2cncnc21]

In some embodiments of Formula (II) or Formulas (II-a) to (II-u′), Q¹ is —CH₂O—; Q⁴ is —CH₂O—; Q² is —O—; Q³ is —O—′ each Xⁿ is independently —OH, —SH, O⁻, or S⁻; each Yⁿ is independently ═O or ═S; B¹ is

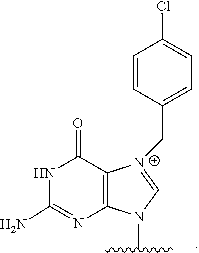 [SMILES: *n1c[n+](Cc2ccc(Cl)cc2)c2c(=O)[nH]c(N)nc21]

In some embodiments of Formula (II) or Formulas (II-a) to (II-u′), B² is adenine. In some embodiments of Formula (II) or Formulas (II-a) to (II-u′), B³ is guanine.

In one aspect, described herein is a mRNA sequence having a 5′-end region motif (Motif (II′):

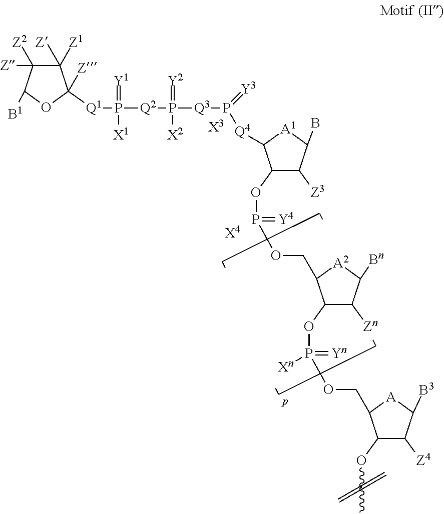

wherein
B¹ is

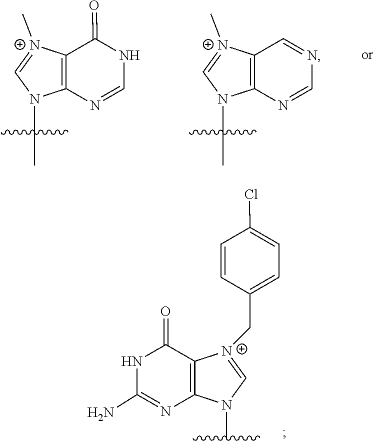 [SMILES: *n1c[n+](C)c2c(=O)[nH]cnc21 ; *n1c[n+](C)c2cncnc21 ; *n1c[n+](Cc2ccc(Cl)cc2)c2c(=O)[nH]c(N)nc21]

each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

In some embodiments of a compound of Motif (II″), (II″-a′), (II″-b′), (II″-c′), (II″-d′), (II″-e′), (II″-f′) (II″-g′), (II″-h′), (II″-i′) (II″-j′), (II″-k′), (II″-l′), (II″-m′), (II″-n′), (II″-o′), (II″-p′), (II″-q′), (II″-r′), (II″-s′), (I″-t′) or (II″-u′), B¹ is

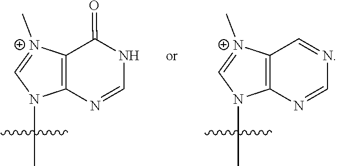 [SMILES: *n1c[n+](C)c2c(=O)[nH]cnc21 ; *n1c[n+](C)c2cncnc21]

In some embodiments, B¹ is

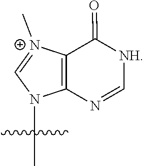 [SMILES: *n1c[n+](C)c2c(=O)[nH]cnc21]

In some embodiments, B¹ is

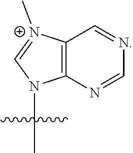 [SMILES: *n1c[n+](C)c2cncnc21]

In some embodiments, B¹ is

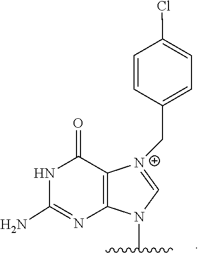 [SMILES: *n1c[n+](Cc2ccc(Cl)cc2)c2c(=O)[nH]c(N)nc21]

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), B² independently a natural, a modified, or an unnatural nucleobase. In some embodiments, B² is adenine. In some embodiments, B² is guanine. In some embodiments, B² is cytosine. In some embodiments, B² is uracil, In some embodiments, B² is thymine, In some embodiments, B² is hypoxanthine. In some embodiments, B² is purine.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-m′), (II″-n′), (II″-o′), (II″-p′), (II″-q′), (II″-r′), (II″-s′), (II″-t′), or (II″-u′), B³ independently a natural, a modified, or an unnatural nucleobase. In some embodiments, B³ is adenine. In some embodiments, B³ is guanine. In some embodiments, B³ is cytosine. In some embodiments, B³ is uracil, In some embodiments, B³ is thymine, In some embodiments, B³ is hypoxanthine. In some embodiments, B³ is purine.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′) (II″-n′), (II″-o′), (II″-p′), (II″-q′), (II″-r′), (II″-s′), (II″-t′), or (II″-u′), Bⁿ independently a natural, a modified, or an unnatural nucleobase. In some embodiments, Bⁿ is adenine. In some embodiments, Bⁿ is guanine. In some embodiments, Bⁿ is cytosine. In some embodiments, Bⁿ is uracil, In some embodiments, Bⁿ is thymine, In some embodiments, Bⁿ is hypoxanthine. In some embodiments, Bⁿ is purine.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Z′ is hydrogen, fluorine, —OH, —SH, —CH₃, —CH₂CH₃, —OCH₃, —NH(CH₃), —NH₂, —NH(C(═O)CH₃), or —SCH₃. In some embodiments, Z′ is hydrogen. In some embodiments, Z′ is fluorine. In some embodiments, Z′ is —OH. In some embodiments, Z′ is —SH. In some embodiments, Z′ is —CH₃. In some embodiments, Z′ is —CH₂CH₃. In some embodiments, Z′ is —OCH₃. In some embodiments, Z′ is —NH(CH₃), In some embodiments, Z′ is —NH₂—. In some embodiments, Z′ is —NH(C(═O)CH₃). In some embodiments, Z′ is —SCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Zⁿ is hydrogen, fluorine, —OH, —SH, —CH₃, —CH₂CH₃, —OCH₃, —NH(CH₃), —NH₂, —NH(C(═O)CH₃), or —SCH₃. In some embodiments, Zⁿ is hydrogen. In some embodiments, Zⁿ is fluorine. In some embodiments, Zⁿ is —OH. In some embodiments, Zⁿ is —SH. In some embodiments, Zⁿ is —CH₃. In some embodiments, Zⁿ is —CH₂CH₃. In some embodiments, Zⁿ is —OCH₃. In some embodiments, Zⁿ is —NH(CH₃), In some embodiments, Zⁿ is —NH₂—. In some embodiments, Zⁿ is —NH(C(═O)CH₃). In some embodiments, Zⁿ is —SCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Z′″ is hydrogen, fluorine, —CH₃, —CH₂CH₃, —OCH₃, or —OCH₂CH₃. In some embodiments, Z′″ is hydrogen. In some embodiments, Z′″ is fluorine. In some embodiments, Z′″ is —CH₃. In some embodiments, Z″′ is —CH₂CH₃. In some embodiments, Z′″ is —OCH₃. In some embodiments, Z′″ is —OCH₂CH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′)(II″-n′) (II″-o′), (II″-p′), (II″-q′), (II″-r′), (II″-s′), (II″-t′), or (II″-u′), Z¹ is hydrogen, fluorine, —OH, —SH, —CH₃, —CH₂CH₃, —OCH₃, —SCH₃, —OCH₂CH₃, —NH₂, NHCH₃, or NHC(═O)CH₃. In some embodiments, Z¹ is hydrogen. In some embodiments, Z¹ is fluorine. In some embodiments, Z¹ is —OH. In some embodiments, Z¹ is —SH. In some embodiments, Z¹ is —CH₃. In some embodiments, Z¹ is —CH₂CH₃. In some embodiments, Z¹ is —OCH₃. In some embodiments, Z¹ is —SCH₃. In some embodiments, Z¹ is —OCH₂CH₃. In some embodiments, Z¹ is —NH₂. In some embodiments, Z¹ is NHCH₃. In some embodiments, Z¹ is NHC(═O)CH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Z² is hydrogen, fluorine, —OH, —SH, —CH₃, —CH₂CH₃, —OCH₃, —SCH₃, —OCH₂CH₃, —NH₂, NHCH₃, or NHC(═O)CH₃. In some embodiments, Z² is hydrogen. In some embodiments, Z² is fluorine. In some embodiments, Z² is —OH. In some embodiments, Z² is —SH. In some embodiments, Z² is —CH₃. In some embodiments, Z² is —CH₂CH₃. In some embodiments, Z² is —OCH₃. In some embodiments, Z² is —SCH₃. In some embodiments, Z² is —OCH₂CH₃. In some embodiments, Z² is —NH₂. In some embodiments, Z² is NHCH₃. In some embodiments, Z² is NHC(═O)CH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Z³ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z³ is hydrogen. In some embodiments, Z³ is fluorine. In some embodiments, Z³ is —OH. In some embodiments, Z³ is —CH₃. In some embodiments, Z³ is —CH₂CH₃. In some embodiments, Z³ is —OCH₃. In some embodiments, Z³ is —NH₂. In some embodiments, Z³ is —NHCH₃. In some embodiments, Z³ is —NH(C(═O)CH₃). In some embodiments, Z³ is —OCH₂CH₃. In some embodiments, Z³ is —OCH₂OCH₃. In some embodiments, Z³ is —OCH₂CH₂CH₃. In some embodiments, Z³ is —OCH(CH₃)₂. In some embodiments, Z³ is —SCH₃. In some embodiments, Z³ is —OCH₂CH₂OCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Z⁴ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z⁴ is hydrogen. In some embodiments, Z⁴ is fluorine. In some embodiments, Z⁴ is —OH. In some embodiments, Z⁴ is —CH₃. In some embodiments, Z⁴ is —CH₂CH₃. In some embodiments, Z⁴ is —OCH₃. In some embodiments, Z⁴ is —NH₂. In some embodiments, Z⁴ is —NHCH₃. In some embodiments, Z⁴ is —NH(C(═O)CH₃). In some embodiments, Z⁴ is —OCH₂CH₃. In some embodiments, Z⁴ is —OCH₂OCH₃. In some embodiments, Z⁴ is —OCH₂CH₂CH₃. In some embodiments, Z⁴ is —OCH(CH₃)₂. In some embodiments, Z⁴ is —SCH₃. In some embodiments, Z⁴ is —OCH₂CH₂OCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Zⁿ is hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Zⁿ is hydrogen. In some embodiments, Zⁿ is fluorine. In some embodiments, Zⁿ is —OH. In some embodiments, Zⁿ is —CH₃. In some embodiments, Zⁿ is —CH₂CH₃. In some embodiments, Zⁿ is —OCH₃. In some embodiments, Zⁿ is —NH₂. In some embodiments, Zⁿ is —NHCH₃. In some embodiments, Zⁿ is —NH(C(═O)CH₃). In some embodiments, Zⁿ is —OCH₂CH₃. In some embodiments, Zⁿ is —OCH₂OCH₃. In some embodiments, Zⁿ is —OCH₂CH₂CH₃. In some embodiments, Zⁿ is —OCH(CH₃)₂. In some embodiments, Zⁿ is —SCH₃. In some embodiments, Zⁿ is —OCH₂CH₂OCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Q¹ is —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH═CH—. In some embodiments, Q¹ is —CH₂—. In some embodiments, Q¹ is —CH₂O—. In some embodiments, Q¹ is —CH₂S—. In some embodiments, Q¹ is —CH₂CH₂—. In some embodiments, Q¹ is —CH₂CF₂—. In some embodiments, Q¹ is —CH₂NH₂—. In some embodiments, Q¹ is —CH₂NH(CH₃)—. In some embodiments, Q¹ is —CH₂N(C(═O)CH₃)—.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Q⁴ is —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH═CH—. In some embodiments, Q⁴ is —CH₂—. In some embodiments, Q⁴ is —CH₂O—. In some embodiments, Q⁴ is —CH₂S—. In some embodiments, Q⁴ is —CH₂CH₂—. In some embodiments, Q⁴ is —CH₂CF₂—. In some embodiments, Q⁴ is —CH₂NH₂—. In some embodiments, Q⁴ is —CH₂NH(CH₃)—. In some embodiments, Q⁴ is —CH₂N(C(═O)CH₃)—.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Q² is —O—, —S—, —CH₂—, —CF₂—, —NH—, —N(CH₃)—, or —N(C(═O)CH₃)—. In some embodiments Q² is —O—. In some embodiments Q² is —S—. In some embodiments Q² is —CH₂—. In some embodiments Q² is —CF₂—. In some embodiments Q² is —NH—. In some embodiments Q² is —N(CH₃)—. In some embodiments Q² is —N(C(═O)CH₃)—.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Q³ is —O—, —S—, —CH₂—, —CF₂—, —NH—, —N(CH₃)—, or —N(C(═O)CH₃)—. In some embodiments Q³ is —O—. In some embodiments Q³ is —S—. In some embodiments Q³ is —CH₂—. In some embodiments Q³ is —CF₂—. In some embodiments Q³ is —NH—. In some embodiments Q³ is —N(CH₃)—. In some embodiments Q³ is —N(C(═O)CH₃)—.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), X¹ is —OH, —SH, —O⁻, —S⁻, —NH2, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X¹ is —OH. In some embodiments, X¹ is —SH. In some embodiments, X¹ is —O⁻. In some embodiments, X¹ is —S⁻. In some embodiments, X¹ is —NH₂. In some embodiments, X¹ is —NHCH₃. In some embodiments, X¹ is —NH(C(═O)CH₃). In some embodiments, X¹ is —CH₃. In some embodiments, X¹ is —CH₂CH₃. In some embodiments, X¹ is —CH₂CH₂CH₃. In some embodiments, X¹ is —CH(CH₃)₂. In some embodiments, X¹ is —OCH₃. In some embodiments, X¹ is —OCH₂CH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), X2 is —OH, —SH, —O⁻, —S⁻, —NH2, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X² is —OH. In some embodiments, X² is —SH. In some embodiments, X² is —O⁻. In some embodiments, X² is —S⁻. In some embodiments, X² is —NH₂. In some embodiments, X² is —NHCH₃. In some embodiments, X² is —NH(C(═O)CH₃). In some embodiments, X² is —CH₃. In some embodiments, X² is —CH₂CH₃. In some embodiments, X² is —CH₂CH₂CH₃. In some embodiments, X² is —CH(CH₃)₂. In some embodiments, X² is —OCH₃. In some embodiments, X² is —OCH₂CH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), X³ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X³ is —OH. In some embodiments, X³ is —SH. In some embodiments, X³ is —O⁻. In some embodiments, X³ is —S⁻. In some embodiments, X³ is —NH₂. In some embodiments, X³ is —NHCH₃. In some embodiments, X³ is —NH(C(═O)CH₃). In some embodiments, X³ is —CH₃. In some embodiments, X³ is —CH₂CH₃. In some embodiments, X³ is —CH₂CH₂CH₃. In some embodiments, X³ is —CH(CH₃)₂. In some embodiments, X³ is —OCH₃. In some embodiments, X³ is —OCH₂CH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), X⁴ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, X⁴ is —OH. In some embodiments, X⁴ is —SH. In some embodiments, X⁴ is —O⁻. In some embodiments, X⁴ is —S⁻. In some embodiments, X⁴ is —NH₂. In some embodiments, X⁴ is —NHCH₃. In some embodiments, X⁴ is —NH(C(═O)CH₃). In some embodiments, X⁴ is —CH₃. In some embodiments, X⁴ is —CH₂CH₃. In some embodiments, X⁴ is —CH₂CH₂CH₃. In some embodiments, X⁴ is —CH(CH₃)₂. In some embodiments, X⁴ is —OCH₃. In some embodiments, X⁴ is —OCH₂CH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Xⁿ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃. In some embodiments, Xⁿ is —OH. In some embodiments, Xⁿ is —SH. In some embodiments, Xⁿ is —O—. In some embodiments, Xⁿ is —S—. In some embodiments, Xⁿ is —NH₂. In some embodiments, Xⁿ is —NHCH₃. In some embodiments, Xⁿ is —NH(C(═O)CH₃). In some embodiments, Xⁿ is —CH₃. In some embodiments, Xⁿ is —CH₂CH₃. In some embodiments, Xⁿ is —CH₂CH₂CH₃. In some embodiments, Xⁿ is —CH(CH₃)₂. In some embodiments, Xⁿ is —OCH₃. In some embodiments, Xⁿ is —OCH₂CH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Y¹ is ═O, ═S, ═NH, or ═NCH₃. In some embodiments, Y¹ is ═O. In some embodiments, Y¹ is ═S. In some embodiments, Y¹ is ═NH. In some embodiments, Y¹ is ═NCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Y² is ═O, ═S, ═NH, or ═NCH₃. In some embodiments, Y² is ═O. In some embodiments, Y² is ═S. In some embodiments, Y² is ═NH. In some embodiments, Y² is ═NCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Y³ is ═O, ═S, ═NH, or ═NCH₃. In some embodiments, Y³ is ═O. In some embodiments, Y³ is ═S. In some embodiments, Y³ is ═NH. In some embodiments, Y³ is ═NCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Y⁴ is ═O, ═S, ═NH, or ═NCH₃. In some embodiments, Y⁴ is ═O. In some embodiments, Y⁴ is ═S. In some embodiments, Y⁴ is ═NH. In some embodiments, Y⁴ is ═NCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), Yⁿ is ═O, ═S, ═NH, or ═NCH₃. In some embodiments, Yⁿ is ═O. In some embodiments, Yⁿ is ═S. In some embodiments, Yⁿ is ═NH. In some embodiments, Yⁿ is ═NCH₃.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), A is —O—, —S—, —CH₂—, —NH—, —N(CH₃)— or —N(C(═O)CH₃)—. In some embodiments, A is —O—. In some embodiments, A is —S—. In some embodiments, A is —CH₂—. In some embodiments, A is —NH—. In some embodiments, A is —N(CH₃)—. In some embodiments, A is —N(C(═O)CH₃)—.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), A¹ is —O—, —S—, —CH₂—, —NH—, —N(CH₃)— or —N(C(═O)CH₃)—. In some embodiments, A¹ is —O—. In some embodiments, A¹ is —S—. In some embodiments, A¹ is —CH₂—. In some embodiments, A¹ is —NH—. In some embodiments, A¹ is —N(CH₃)—. In some embodiments, A¹ is —N(C(═O)CH₃)—.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), A2 is —O—, —S—, —CH₂—, —NH—, —N(CH₃)— or —N(C(═O)CH₃)—. In some embodiments, A² is —O—. In some embodiments, A² is —S—. In some embodiments, A² is —CH₂—. In some embodiments, A² is —NH—. In some embodiments, A² is —N(CH₃)—. In some embodiments, A² is —N(C(═O)CH₃)—.

In some embodiments of a compound of Motif (II″), (II″-a′), (II″-b′), (II″-c′), (II″-d′), (II″-e′), (II″-f′), (II″-g′), (II″-h′), (II″-i′), (II″-j′), (II″-k′), (II″-l′), (II″-m′), (II″-n′), (II″-o′), (II″-p′), (II″-q′), (II″-r′), (II″-s′), (II″-t′), or (II″-u′), p is 0, 1, 2, 3, 4, 5 or 6. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments, B¹ is

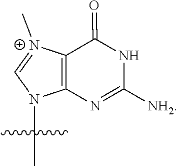 [SMILES: *n1c[n+](C)c2c(=O)[nH]c(N)nc21]

In some embodiments, each B² and B³ is independently adenine or guanine. In some embodiments, B² is adenine. In some embodiments, B³ is guanine.

In some embodiments, B¹ is

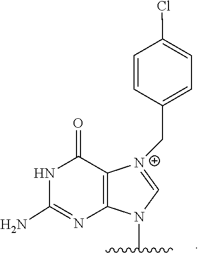 [SMILES: *n1c[n+](Cc2ccc(Cl)cc2)c2c(=O)[nH]c(N)nc21]

In some embodiments, each B² and B³ is independently adenine or guanine. In some embodiments, B² is adenine. In some embodiments, B³ is guanine.

In some embodiments, each Z¹, Z², Z³, and Z⁴ is independently —OH or —OCH₃. In some embodiments, Z³ is —OCH₃. In some embodiments, Z¹ is —OCH₃. In some embodiments Z² is —OH. In some embodiments, Z¹, Z², and Z⁴ are —OH and Z³ is —OCH₃. In some embodiments, Z¹ and Z³ are —OCH₃ and Z² and Z⁴ are —OH. In some embodiments, Z′, Z″, and Z′″ are hydrogen.

In some embodiments, Q¹ and Q⁴ are —OCH₂—. In some embodiments Q² and Q³ are —O—.

In some embodiments, X¹, X², X³, and X⁴ are —O—. In some embodiments, X¹, X², X³, and X⁴ are —S—. In some embodiments, X¹, X³, X⁴ are —O— and X² is —S—.

In some embodiments, Y¹, Y², Y³, and Y⁴ are ═O. In some embodiments, Y¹, Y², Y³, and Y⁴ are ═S. In some embodiments, Y¹, Y³, and Y⁴ are ═O and Y² is ═S. In some embodiments, Y¹, Y², Y³ are ═O and Y⁴ is ═S.

In some embodiments, A, A¹, and A² are —O—.

In some embodiments, p is 0.
one aspect, described herein is an IVT mRNA sequence initiator of Formula (I) or a salt or solvate thereof:

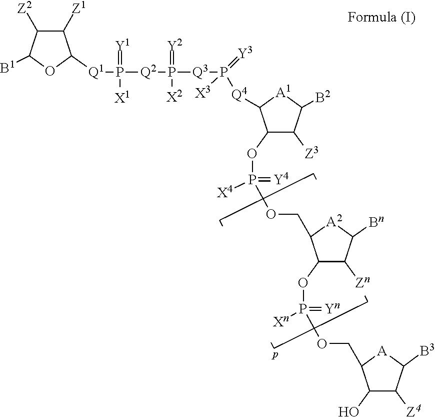

wherein
B¹ is

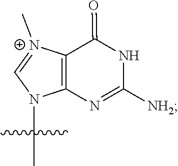 [SMILES: *n1c[n+](C)c2c(=O)[nH]c(N)nc21]

B² is a modified or unnatural nucleobase;
   each B³, and Bⁿ is independently a natural, a modified, or an unnatural nucleobase;
each Z¹ and Z² is independently hydrogen, fluorine, —OH, —SH, —CH₃, —CH₂CH₃, —OCH₃, —OCH₂CH₃, —SCH₃, —NH₂, NHCH₃, or NHC(═O)CH₃;
each Z³, Z⁴, and Zⁿ is independently hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

In some embodiments, the IVT mRNA sequence initiator has a structure of Formula (III-a):

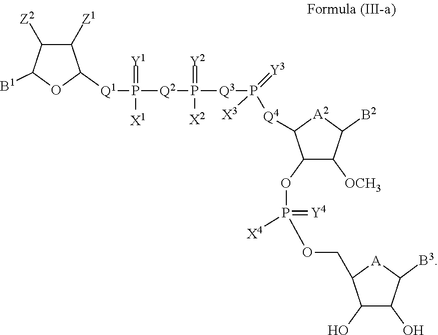

In some embodiments of a compound of Formula (III) or (III-a). Z¹ is hydrogen, fluorine, —OH, —SH, —CH₃, —CH₂CH₃, —OCH₃, —OCH₂CH₃, —SCH₃, —NH₂, NHCH₃, or NHC(═O)CH₃. In some embodiments, Z¹ is hydrogen. In some embodiments, Z¹ is F. In some embodiments, Z¹ is —OH. In some embodiments, Z¹ is —SH. In some embodiments, Z¹ is —CH₃. In some embodiments, Z¹ is —CH₂CH₃. In some embodiments, Z¹ is —OCH₃. In some embodiments, Z¹ is —OCH₂CH₃. In some embodiments, Z¹ is —SCH₃. In some embodiments, Z¹ is —NH₂. In some embodiments, Z¹ is NHCH₃. In some embodiments, Z¹ is NHC(═O)CH₃.

In some embodiments of a compound of Formula (III) or (III-a), Z² is hydrogen, fluorine, —OH, —SH, —CH₃, —CH₂CH₃, —OCH₃, —OCH₂CH₃, —SCH₃, —NH₂, NHCH₃, or NHC(═O)CH₃. In some embodiments, Z² is hydrogen. In some embodiments, Z² is F. In some embodiments, Z² is —OH. In some embodiments, Z² is —SH. In some embodiments, Z² is —CH₃. In some embodiments, Z² is —CH₂CH₃. In some embodiments, Z² is —OCH₃. In some embodiments, Z² is —OCH₂CH₃. In some embodiments, Z² is —SCH₃. In some embodiments, Z² is —NH₂. In some embodiments, Z² is NHCH₃. In some embodiments, Z² is NHC(═O)CH₃.

In some embodiments of a compound of Formula (III) or (III-a), Z³ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z³ is hydrogen. In some embodiments, Z³ is fluorine. In some embodiments, Z³ is —OH. In some embodiments, Z³ is —SH. In some embodiments, Z³ is —CH₃. In some embodiments, Z³ is —CH₂CH₃. In some embodiments, Z³ is —OCH₂OCH₃. In some embodiments, Z³ is —OCH₂CH₂CH₃. In some embodiments, Z³ is —OCH(CH₃)₂. In some embodiments, Z³ is —SCH₃. In some embodiments, Z³ is —OCH₂CH₂OCH₃.

In some embodiments of a compound of (III) or (III-a), Z⁴ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z⁴ is hydrogen. In some embodiments, Z⁴ is fluorine. In some embodiments, Z⁴ is —OH. In some embodiments, Z⁴ is —SH. In some embodiments, Z⁴ is —CH₃. In some embodiments, Z⁴ is —CH₂CH₃. In some embodiments, Z⁴ is —OCH₂OCH₃. In some embodiments, Z⁴ is —OCH₂CH₂CH₃. In some embodiments, Z⁴ is —OCH(CH₃)₂. In some embodiments, Z⁴ is —SCH₃. In some embodiments, Z⁴ is —OCH₂CH₂OCH₃.

In some embodiments of a compound of Formula (III) or (III-a), Zⁿ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH2CH₂OCH3. In some embodiments, Zⁿ is hydrogen. In some embodiments, Zⁿ is fluorine. In some embodiments, Zⁿ is —OH. In some embodiments, Zⁿ is —SH. In some embodiments, Zⁿ is —CH₃. In some embodiments, Zⁿ is —CH₂CH₃. In some embodiments, Zⁿ is —OCH₂OCH₃. In some embodiments, Zⁿ is —OCH₂CH₂CH₃. In some embodiments, Zⁿ is —OCH(CH₃)₂. In some embodiments, Zⁿ is —SCH₃. In some embodiments, Zⁿ is —OCH₂CH₂OCH₃.

In some embodiments of a compound of Formula (III) or (III-a), B² is a modified, or an unnatural nucleobase. In some embodiments B² is a modified guanine. In some embodiments B² is a modified adenine. In some embodiments, B² is a modified cytosine. In some embodiments, B² is a modified uracil, In some embodiments, B² is a modified thymine, In some embodiments, B² is a modified hypoxanthine. In some embodiments, B² is a modified purine. In some embodiments, B² is 2-aminoadenine. In some embodiments, B² is

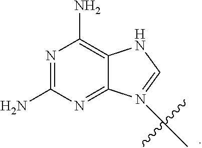

In some embodiments of a compound of Formula (III) or (III-a), B³ is independently a natural, a modified, or an unnatural nucleobase. In some embodiments, B³ is guanine. In some embodiments, B³ is adenine. In some embodiments, B³ is cytosine. In some embodiments, B³ is uracil, In some embodiments, B³ is thymine, In some embodiments, B³ is hypoxanthine. In some embodiments, B³ is purine.

In some embodiments of a compound of Formula (III) or (III-a), Bⁿ is independently a natural, a modified, or an unnatural nucleobase. In some embodiments, Bⁿ is guanine. In some embodiments, Bⁿ is adenine. In some embodiments, Bⁿ is cytosine. In some embodiments, Bⁿ is uracil, In some embodiments, Bⁿ is thymine, In some embodiments, Bⁿ is hypoxanthine. In some embodiments, Bⁿ is purine.

In some embodiments of a compound of Formula (III) or (III-a) B² is a modified adenine. In some embodiments, B³ is guanine.

In some embodiments of a compound of Formula (III) or (III-a), Q¹ is —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH₂—. In some embodiments, Q¹ is —CH═CH—. In some embodiments, Q¹ is —CH₂O—. In some embodiments, Q¹ is —CH₂S—. In some embodiments, Q¹ is —CH₂CH₂—. In some embodiments, Q¹ is —CH₂CF₂—. In some embodiments, Q¹ is —CH₂NH₂—. In some embodiments, Q¹ is —CH₂NH(CH₃)—. In some embodiments, Q¹ is —CH₂N(C(═O)CH₃)—.

In some embodiments of a compound of Formula (III) or (III-a), Q⁴ is —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q⁴ is —CH₂—. In some embodiments, Q⁴ is —CH═CH—. In some embodiments, Q⁴ is —CH₂O—. In some embodiments, Q⁴ is —CH₂S—. In some embodiments, Q⁴ is —CH₂CH₂—. In some embodiments, Q⁴ is —CH₂CF₂—. In some embodiments, Q⁴ is —CH₂NH₂—. In some embodiments, Q⁴ is —CH₂NH(CH₃)—. In some embodiments, Q⁴ is —CH₂N(C(═O)CH₃)—.

In some embodiments of a compound of Formula (III) or (III-a), Q² is —O—, —S—, —CH₂—, —CF₂—, —NH—, —N(CH₃)—, or —N(C(═O)CH₃)—. In some embodiments, Q² is —O—. In some embodiments, Q² is —S—. In some embodiments, Q² is —CH₂—. In some embodiments, Q² is —CF₂—. In some embodiments, Q² is —NH—. In some embodiments, Q² is —N(CH₃)—. In some embodiments, Q² is —N(C(═O)CH₃).

In some embodiments of a compound of Formula (III) or (III-a), Q³ is —O—, —S—, —CH₂—, —CF₂—, —NH—, —N(CH₃)—, or —N(C(═O)CH₃)—. In some embodiments, Q³ is —O—. In some embodiments, Q³ is —S—. In some embodiments, Q³ is —CH₂—. In some embodiments, Q³ is —CF₂—. In some embodiments, Q³ is —NH—. In some embodiments, Q³ is —N(CH₃)—. In some embodiments, Q³ is —N(C(═O)CH₃).

In some embodiments of a compound of Formula (III) or (III-a), X¹ is —OH, —SH, —O⁻, —S⁻—NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X¹ is —OH. In some embodiments, X¹ is —SH. In some embodiments, X¹ is —O—. In some embodiments, X¹ is —S—. In some embodiments, X¹ is —NH₂. In some embodiments, X¹ is —NHCH₃. In some embodiments, X¹ is —NH(C(═O)CH₃). In some embodiments, X¹ is —CH₃. In some embodiments, X¹ is —CH₂CH₃. In some embodiments, X¹ is —CH₂CH₂CH₃. In some embodiments, X¹ is —CH(CH₃)₂. In some embodiments, X¹ is —OCH₃. In some embodiments, X¹ is —OCH₂CH₃.

In some embodiments of a compound of Formula (III) or (III-a), X² is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X² is —OH. In some embodiments, X² is —SH. In some embodiments, X² is —O⁻. In some embodiments, X² is —S⁻. In some embodiments, X² is —NH₂. In some embodiments, X² is —NHCH₃. In some embodiments, X² is —NH(C(═O)CH₃). In some embodiments, X² is —CH₃. In some embodiments, X² is —CH₂CH₃. In some embodiments, X² is —CH₂CH₂CH₃. In some embodiments, X² is —CH(CH₃)₂. In some embodiments, X² is —OCH₃. In some embodiments, X² is —OCH₂CH₃.

In some embodiments of a compound of Formula (III) or (III-a), X³ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X³ is —OH. In some embodiments, X³ is —SH. In some embodiments, X³ is —O⁻. In some embodiments, X³ is —S⁻. In some embodiments, X³ is —NH₂. In some embodiments, X³ is —NHCH₃. In some embodiments, X³ is —NH(C(═O)CH₃). In some embodiments, X³ is —CH₃. In some embodiments, X³ is —CH₂CH₃. In some embodiments, X³ is —CH₂CH₂CH₃. In some embodiments, X³ is —CH(CH₃)₂. In some embodiments, X³ is —OCH₃. In some embodiments, X³ is —OCH₂CH₃.

In some embodiments of a compound of Formula (III) or (III-a), X⁴ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X⁴ is —OH. In some embodiments, X⁴ is —SH. In some embodiments, X⁴ is —O—. In some embodiments, X⁴ is —S—. In some embodiments, X⁴ is —NH₂. In some embodiments, X⁴ is —NHCH₃. In some embodiments, X⁴ is —NH(C(═O)CH₃). In some embodiments, X⁴ is —CH₃. In some embodiments, X⁴ is —CH₂CH₃. In some embodiments, X⁴ is —CH₂CH₂CH₃. In some embodiments, X⁴ is —CH(CH₃)₂. In some embodiments, X⁴ is —OCH₃. In some embodiments, X⁴ is —OCH₂CH₃.

In some embodiments of a compound of Formula (III) or (III-a), Xⁿ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, Xⁿ is —OH. In some embodiments, Xⁿ is —SH. In some embodiments, Xⁿ is —O⁻. In some embodiments, Xⁿ is —S⁻. In some embodiments, Xⁿ is —NH₂. In some embodiments, Xⁿ is —NHCH₃. In some embodiments, Xⁿ is —NH(C(═O)CH₃). In some embodiments, Xⁿ is —CH₃. In some embodiments, Xⁿ is —CH₂CH₃. In some embodiments, Xⁿ is —CH₂CH₂CH₃. In some embodiments, Xⁿ is —CH(CH₃)₂. In some embodiments, Xⁿ is —OCH₃. In some embodiments, Xⁿ is —OCH₂CH₃.

In some embodiments of a compound of (III) or (III-a), Y¹ is ═O, ═S, ═NH, or ═NCH₃. In some embodiments, Y¹ is ═O. In some embodiments, Y¹ is ═S. In some embodiments, Y¹ is ═NH. In some embodiments, Y¹ is ═NHCH₃.

In some embodiments of a compound of Formula (III) or (III-a), Y² is ═O, ═S, ═NH, or ═NCH₃. In some embodiments, Y² is ═O. In some embodiments, Y² is ═S. In some embodiments, Y² is ═NH. In some embodiments, Y² is ═NHCH₃.

In some embodiments of a compound of (III) or (III-a), Y³ is ═O, ═S, ═NH, or ═NCH₃. In some embodiments, Y³ is ═O. In some embodiments, Y³ is ═S. In some embodiments, Y³ is ═NH. In some embodiments, Y³ is ═NHCH₃.

In some embodiments of a compound of Formula (III) or (III-a), Y⁴ is ═O, ═S, ═NH, or ═NCH₃. In some embodiments, Y⁴ is ═O. In some embodiments, Y⁴ is ═S. In some embodiments, Y⁴ is ═NH. In some embodiments, Y⁴ is ═NHCH₃.

In some embodiments of a compound of Formula (III) or (III-a), Yⁿ is ═O, ═S, ═NH, or ═NCH₃. In some embodiments, Yⁿ is ═O. In some embodiments, Yⁿ is ═S. In some embodiments, Yⁿ is ═NH. In some embodiments, Yⁿ is ═NHCH₃.

In some embodiments of a compound of Formula (III) or (III-a), A is —O—, —S—, —CH₂—, —NH—, —N(CH₃)— or —N(C(═O)CH₃)—. In some embodiments, A is —O—. In some embodiments, A is —S—. In some embodiments, A is —CH₂—. In some embodiments, A is —NH—. In some embodiments, A is —N(CH₃)—. In some embodiments, A is —N(C(═O)CH₃)—.

In some embodiments of a compound of (III) or (III-a), A¹ is —O—, —S—, —CH₂—, —NH—, —N(CH₃)— or —N(C(═O)CH₃)—. In some embodiments, A¹ is —O—. In some embodiments, A¹ is —S—. In some embodiments, A¹ is —CH₂—. In some embodiments, A¹ is —NH—. In some embodiments, A¹ is —N(CH₃)—. In some embodiments, A¹ is —N(C(═O)CH₃)—.

In some embodiments of a compound of (III) or (III-a), A² is —O—, —S—, —CH₂—, —NH—, —N(CH₃)— or —N(C(═O)CH₃)—. In some embodiments, A² is —O—. In some embodiments, A² is —S—. In some embodiments, A² is —CH₂—. In some embodiments, A² is —NH—. In some embodiments, A² is —N(CH₃)—. In some embodiments, A² is —N(C(═O)CH₃)—.

In some embodiments of a compound of Formula (III) or (III-a), p is 0, 1, 2, 3, 4, 5 or 6. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments of a compound of Formula (III) or (III-a), at least one of Z¹, Z², Z³, Z⁴, and Zⁿ is —OCH₃. In some embodiments, Z³ is —OCH₃. In some embodiments, Z³ and Z¹ are —OCH₃. In some embodiments of a compound of Formula (I), at least one of Z¹, Z², Z³, Z⁴, and Zⁿ is —OH. In some embodiments, Z¹, Z², and Z⁴ are —OH. In some embodiments, Z² and Z⁴ are —OH.

In some embodiments of a compound of Formula (III) or (III-a), at least one of Q¹, Q², Q³, and Q⁴ is —OCH₃. In some embodiments of a compound of Formula (I), at least one of Q¹, Q², Q³, and Q⁴ is —O—. In some embodiments, Q¹ and Q⁴ are —OCH₃. In some embodiments, Q² and Q³ are —O—.

In some embodiments of a compound o Formula (III) or (III-a), at least one of Y¹, Y², Y³, Y⁴, and Yⁿ is ═O. In some embodiments, Y¹, Y², Y³, and Y⁴ is are ═O. In some embodiments, Y¹, Y², Y³, Y⁴, and Yⁿ is ═S.

In some embodiments of a compound of Formula (III) or (III-a), at least one of X¹, X², X³, X⁴, and Xⁿ is —O—. In Some embodiments, X¹, X², X³, and X⁴ are —O—.

In some embodiments of a compound of (III) or (III-a), In some embodiments of a compound of Formula (I), at least one of A, A¹, and A² is —O—. In some embodiments, A, A¹, and A² are —O—.

In one aspect, described herein is a mRNA sequence having a 5′-end region motif of Motif (III′):

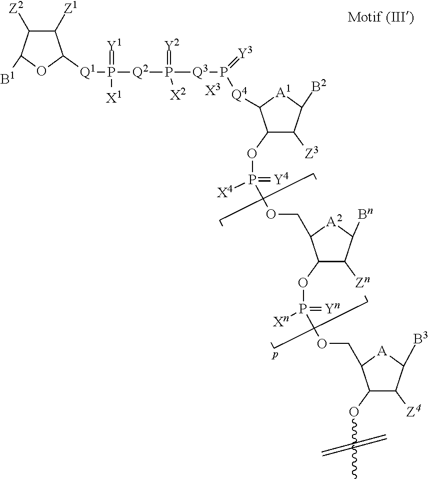

wherein
B¹ is

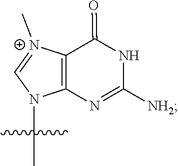 [SMILES: *n1c[n+](C)c2c(=O)[nH]c(N)nc21]

B² is a modified or unnatural nucleobase;
each B³, and Bⁿ is independently a natural, a modified, or an unnatural nucleobase;
each Z¹ and Z² is independently hydrogen, fluorine, —OH, —SH, —CH₃, —CH₂CH₃, —OCH₃, —OCH₂CH₃, —SCH₃, —NH₂, NHCH₃, or NHC(═O)CH₃;
each Z³, Z⁴, and Zⁿ is independently hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

In some embodiments, the mRNA sequence having a 5′-end region motif has a structure of Motif (III′-a):

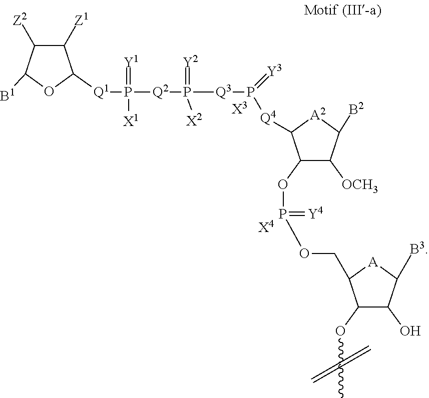

In some embodiments of a compound of Motif (III′) or (III′-a), Z¹ is hydrogen, fluorine, —OH, —SH, —CH₃, —CH₂CH₃, —OCH₃, —OCH₂CH₃, —SCH₃, —NH₂, NHCH₃, or NHC(═O)CH₃. In some embodiments, Z¹ is hydrogen. In some embodiments, Z¹ is F. In some embodiments, Z¹ is —OH. In some embodiments, Z¹ is —SH. In some embodiments, Z¹ is —CH₃. In some embodiments, Z¹ is —CH₂CH₃. In some embodiments, Z¹ is —OCH₃. In some embodiments, Z¹ is —OCH₂CH₃. In some embodiments, Z¹ is —SCH₃. In some embodiments, Z¹ is —NH₂. In some embodiments, Z¹ is NHCH₃. In some embodiments, Z¹ is NHC(═O)CH₃.

In some embodiments of a compound of Motif (III′) or (III′-a), Z² is hydrogen, fluorine, —OH, —SH, —CH₃, —CH₂CH₃, —OCH₃, —OCH₂CH₃, —SCH₃, —NH₂, NHCH₃, or NHC(═O)CH₃. In some embodiments, Z² is hydrogen. In some embodiments, Z² is F. In some embodiments, Z² is —OH. In some embodiments, Z² is —SH. In some embodiments, Z² is —CH₃. In some embodiments, Z² is —CH₂CH₃. In some embodiments, Z² is —OCH₃. In some embodiments, Z² is —OCH₂CH₃. In some embodiments, Z² is —SCH₃. In some embodiments, Z² is —NH₂. In some embodiments, Z² is NHCH₃. In some embodiments, Z² is NHC(═O)CH₃.

In some embodiments of a compound of Motif (III′) or (III′-a), Z³ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Z³ is hydrogen. In some embodiments, Z³ is fluorine. In some embodiments, Z³ is —OH. In some embodiments, Z³ is —SH. In some embodiments, Z³ is —CH₃. In some embodiments, Z³ is —CH₂CH₃. In some embodiments, Z³ is —OCH₂OCH₃. In some embodiments, Z³ is —OCH₂CH₂CH₃. In some embodiments, Z³ is —OCH(CH₃)₂. In some embodiments, Z³ is —SCH₃. In some embodiments, Z³ is —OCH₂CH₂OCH₃.

In some embodiments of a compound of Motif (III′) or (III′-a), Z⁴ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH2CH₂OCH3. In some embodiments, Z⁴ is hydrogen. In some embodiments, Z⁴ is fluorine. In some embodiments, Z⁴ is —OH. In some embodiments, Z⁴ is —SH. In some embodiments, Z⁴ is —CH₃. In some embodiments, Z⁴ is —CH₂CH₃. In some embodiments, Z⁴ is —OCH₂OCH₃. In some embodiments, Z⁴ is —OCH₂CH₂CH₃. In some embodiments, Z⁴ is —OCH(CH₃)₂. In some embodiments, Z⁴ is —SCH₃. In some embodiments, Z⁴ is —OCH₂CH₂OCH₃.

In some embodiments of a compound of Motif (III′) or (III′-a), Zⁿ is hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃. In some embodiments, Zⁿ is hydrogen. In some embodiments, Zⁿ is fluorine. In some embodiments, Zⁿ is —OH. In some embodiments, Zⁿ is —SH. In some embodiments, Zⁿ is —CH₃. In some embodiments, Zⁿ is —CH₂CH₃. In some embodiments, Zⁿ is —OCH₂OCH₃. In some embodiments, Zⁿ is —OCH₂CH₂CH₃. In some embodiments, Zⁿ is —OCH(CH₃)₂. In some embodiments, Zⁿ is —SCH₃. In some embodiments, Zⁿ is —OCH₂CH₂OCH₃.

In some embodiments of a compound of Motif (III′) or (III′-a), B² is a modified, or an unnatural nucleobase. In some embodiments B² is a modified guanine. In some embodiments B² is a modified adenine. In some embodiments, B² is a modified cytosine. In some embodiments, B² is a modified uracil, In some embodiments, B² is a modified thymine, In some embodiments, B² is a modified hypoxanthine. In some embodiments, B² is a modified purine. In some embodiments, B² is 2-aminoadenine. In some embodiments, B² is

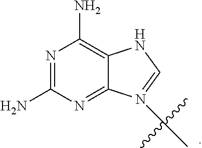

In some embodiments of a compound of Motif (III′) or (III′-a), B³ is independently a natural, a modified, or an unnatural nucleobase. In some embodiments, B³ is guanine. In some embodiments, B³ is adenine. In some embodiments, B³ is cytosine. In some embodiments, B³ is uracil, In some embodiments, B³ is thymine, In some embodiments, B³ is hypoxanthine. In some embodiments, B³ is purine.

In some embodiments of a compound of Motif(III′) or (III′-a), Bⁿ is independently a natural, a modified, or an unnatural nucleobase. In some embodiments, Bⁿ is guanine. In some embodiments, Bⁿ is adenine. In some embodiments, Bⁿ is cytosine. In some embodiments, Bⁿ is uracil, In some embodiments, Bⁿ is thymine, In some embodiments, Bⁿ is hypoxanthine. In some embodiments, Bⁿ is purine.

In some embodiments of a compound of Motif (III′) or (III′-a), at least one of B², B³, and Bⁿ is adenine. In some embodiments, at least one of B², B³, and Bⁿ is guanine. In some embodiments B² is a modified adenine. In some embodiments, B³ is adenine.

In some embodiments of a compound of Motif (III′) or (III′-a), Q¹ is —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q¹ is —CH₂—. In some embodiments, Q¹ is —CH═CH—. In some embodiments, Q¹ is —CH₂O—. In some embodiments, Q¹ is —CH₂S—. In some embodiments, Q¹ is —CH₂CH₂—. In some embodiments, Q¹ is —CH₂CF₂—. In some embodiments, Q¹ is —CH₂NH₂—. In some embodiments, Q¹ is —CH₂NH(CH₃)—. In some embodiments, Q¹ is —CH₂N(C(═O)CH₃)—.

In some embodiments of a compound of Motif (III′) or (III′-a), Q⁴ is —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—. In some embodiments, Q⁴ is —CH₂—. In some embodiments, Q⁴ is —CH═CH—. In some embodiments, Q⁴ is —CH₂O—. In some embodiments, Q⁴ is —CH₂S—. In some embodiments, Q⁴ is —CH₂CH₂—. In some embodiments, Q⁴ is —CH₂CF₂—. In some embodiments, Q⁴ is —CH₂NH₂—. In some embodiments, Q⁴ is —CH₂NH(CH₃)—. In some embodiments, Q⁴ is —CH₂N(C(═O)CH₃)—.

In some embodiments of a compound of Motif (III′) or (III′-a), Q² is —O—, —S—, —CH₂—, —CF₂—, —NH—, —N(CH₃)—, or —N(C(═O)CH₃)—. In some embodiments, Q² is —O—. In some embodiments, Q² is —S—. In some embodiments, Q² is —CH₂—. In some embodiments, Q² is —CF₂—. In some embodiments, Q² is —NH—. In some embodiments, Q² is —N(CH₃)—. In some embodiments, Q² is —N(C(═O)CH₃).

In some embodiments of a compound of Motif (III′) or (III′-a), Q³ is —O—, —S—, —CH₂—, —CF₂—, —NH—, —N(CH₃)—, or —N(C(═O)CH₃)—. In some embodiments, Q³ is —O—. In some embodiments, Q³ is —S—. In some embodiments, Q³ is —CH₂—. In some embodiments, Q³ is —CF₂—. In some embodiments, Q³ is —NH—. In some embodiments, Q³ is —N(CH₃)—. In some embodiments, Q³ is —N(C(═O)CH₃).

In some embodiments of a compound of Motif (III′) or (III′-a), X¹ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X¹ is —OH. In some embodiments, X¹ is —SH. In some embodiments, X¹ is —O⁻. In some embodiments, X¹ is —S⁻. In some embodiments, X¹ is —NH₂. In some embodiments, X¹ is —NHCH₃. In some embodiments, X¹ is —NH(C(═O)CH₃). In some embodiments, X¹ is —CH₃. In some embodiments, X¹ is —CH₂CH₃. In some embodiments, X¹ is —CH₂CH₂CH₃. In some embodiments, X¹ is —CH(CH₃)₂. In some embodiments, X¹ is —OCH₃. In some embodiments, X¹ is —OCH₂CH₃.

In some embodiments of a compound of Motif (III′) or (III′-a), X² is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X² is —OH. In some embodiments, X² is —SH. In some embodiments, X² is —O⁻. In some embodiments, X² is —S⁻. In some embodiments, X² is —NH₂. In some embodiments, X² is —NHCH₃. In some embodiments, X² is —NH(C(═O)CH₃). In some embodiments, X² is —CH₃. In some embodiments, X² is —CH₂CH₃. In some embodiments, X² is —CH₂CH₂CH₃. In some embodiments, X² is —CH(CH₃)₂. In some embodiments, X² is —OCH₃. In some embodiments, X² is —OCH₂CH₃.

In some embodiments of a compound of Motif (III′) or (III′-a), X³ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X³ is —OH. In some embodiments, X³ is —SH. In some embodiments, X³ is —O⁻. In some embodiments, X³ is —S⁻. In some embodiments, X³ is —NH₂. In some embodiments, X³ is —NHCH₃. In some embodiments, X³ is —NH(C(═O)CH₃). In some embodiments, X³ is —CH₃. In some embodiments, X³ is —CH₂CH₃. In some embodiments, X³ is —CH₂CH₂CH₃. In some embodiments, X³ is —CH(CH₃)₂. In some embodiments, X³ is —OCH₃. In some embodiments, X³ is —OCH₂CH₃.

In some embodiments of a compound of Motif (III′) or (III′-a), X⁴ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, X⁴ is —OH. In some embodiments, X⁴ is —SH. In some embodiments, X⁴ is —O⁻. In some embodiments, X⁴ is —S⁻. In some embodiments, X⁴ is —NH₂. In some embodiments, X⁴ is —NHCH₃. In some embodiments, X⁴ is —NH(C(═O)CH₃). In some embodiments, X⁴ is —CH₃. In some embodiments, X⁴ is —CH₂CH₃. In some embodiments, X⁴ is —CH₂CH₂CH₃. In some embodiments, X⁴ is —CH(CH₃)₂. In some embodiments, X⁴ is —OCH₃. In some embodiments, X⁴ is —OCH₂CH₃.

In some embodiments of a compound of Motif (III′) or (III′-a), Xⁿ is —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃. In some embodiments, Xⁿ is —OH. In some embodiments, Xⁿ is —SH. In some embodiments, Xⁿ is —O⁻. In some embodiments, Xⁿ is —S⁻. In some embodiments, Xⁿ is —NH₂. In some embodiments, Xⁿ is —NHCH₃. In some embodiments, Xⁿ is —NH(C(═O)CH₃). In some embodiments, Xⁿ is —CH₃. In some embodiments, Xⁿ is —CH₂CH₃. In some embodiments, Xⁿ is —CH₂CH₂CH₃. In some embodiments, Xⁿ is —CH(CH₃)₂. In some embodiments, Xⁿ is —OCH₃. In some embodiments, Xⁿ is —OCH₂CH₃.

In some embodiments of a compound of Motif (III′) or (III′-a), Y¹ is ═O, ═S, ═NH, or ═NCH₃. In some embodiments, Y¹ is ═O. In some embodiments, Y¹ is ═S. In some embodiments, Y¹ is ═NH. In some embodiments, Y¹ is ═NHCH₃.

In some embodiments of a compound of Motif (III′) or (III′-a), Y² is ═O, ═S, ═NH, or ═NCH₃. In some embodiments, Y² is ═O. In some embodiments, Y² is ═S. In some embodiments, Y² is ═NH. In some embodiments, Y² is ═NHCH₃.

In some embodiments of a compound of Motif(III′) or (III′-a), Y³ is ═O, ═S, ═NH, or ═NCH₃. In some embodiments, Y³ is ═O. In some embodiments, Y³ is ═S. In some embodiments, Y³ is ═NH. In some embodiments, Y³ is ═NHCH₃.

In some embodiments of a compound of Motif (III′) or (III′-a), Y⁴ is ═O, ═S, ═NH, or ═NCH₃. In some embodiments, Y⁴ is ═O. In some embodiments, Y⁴ is ═S. In some embodiments, Y⁴ is ═NH. In some embodiments, Y⁴ is ═NHCH₃.

In some embodiments of a compound of Motif (III′) or (III′-a), Yⁿ is ═O, ═S, ═NH, or ═NCH₃. In some embodiments, Yⁿ is ═O. In some embodiments, Yⁿ is ═S. In some embodiments, Yⁿ is ═NH. In some embodiments, Yⁿ is ═NHCH₃.

In some embodiments of a compound of Motif (III′) or (III′-a), A is —O—, —S—, —CH₂—, —NH—, —N(CH₃)— or —N(C(═O)CH₃)—. In some embodiments, A is —O—. In some embodiments, A is —S—. In some embodiments, A is —CH₂—. In some embodiments, A is —NH—. In some embodiments, A is —N(CH₃)—. In some embodiments, A is —N(C(═O)CH₃)—.

In some embodiments of a compound of Motif (III′) or (III′-a), A¹ is —O—, —S—, —CH₂—, —NH—, —N(CH₃)— or —N(C(═O)CH₃)—. In some embodiments, A¹ is —O—. In some embodiments, A¹ is —S—. In some embodiments, A¹ is —CH₂—. In some embodiments, A¹ is —NH—. In some embodiments, A¹ is —N(CH₃)—. In some embodiments, A¹ is —N(C(═O)CH₃)—.

In some embodiments of a compound of Motif (III′) or (III′-a), A² is —O—, —S—, —CH₂—, —NH—, —N(CH₃)— or —N(C(═O)CH₃)—. In some embodiments, A² is —O—. In some embodiments, A² is —S—. In some embodiments, A² is —CH₂—. In some embodiments, A² is —NH—. In some embodiments, A² is —N(CH₃)—. In some embodiments, A² is —N(C(═O)CH₃)—.

In some embodiments of a compound of Motif (III′) or (III′-a), p is 0, 1, 2, 3, 4, 5 or 6. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments of a compound of Motif (III′) or (III′-a), at least one of Z¹, Z², Z³, Z⁴, and Zⁿ is —OCH₃. In some embodiments, Z³ is —OCH₃. In some embodiments, Z³ and Z¹ are —OCH₃. In some embodiments of a compound of Motif (I′), at least one of Z¹, Z², Z³, Z⁴, and Zⁿ is —OH. In some embodiments, Z¹, Z², and Z⁴ are —OH. In some embodiments, Z² and Z⁴ are —OH.

In some embodiments of a compound of Motif (III′) or (III′-a), at least one of Q¹, Q², Q³, and Q⁴ is —OCH₃. In some embodiments of a compound of Formula (I), at least one of Q¹, Q², Q³, and Q⁴ is —O—. In some embodiments, Q¹ and Q⁴ are —OCH₃. In some embodiments, Q² and Q³ are —O—.

In some embodiments of a compound o Motif (III′) or (III′-a), at least one of Y¹, Y², Y³, Y⁴ and Yⁿ is ═O. In some embodiments, Y¹, Y², Y³, and Y⁴ is are ═O. In some embodiments, Y¹, Y², Y³, Y⁴, and Yⁿ is ═S. In some embodiments, Y² is ═S. In some embodiments, Y⁴ is ═S.

In some embodiments of a compound of Motif (III′) or (III′-a), at least one of X¹, X², X³, X⁴, and Xⁿ is —O⁻. In Some embodiments, X¹, X², X³, and X⁴ are —O⁻. In some embodiments, X¹ is —S⁻. In some embodiments, X² is —S⁻. In some embodiments, X³ is —S⁻. In some embodiments, X⁴ is —S⁻.

In some embodiments of a compound of Motif (III′) or (III′-a), In some embodiments of a compound of Motif (I′), at least one of A, A¹, and A² is —O—. In some embodiments, A, A¹, and A² are —O—.

In certain embodiments the sequence initiator compound is as described in Table 1.

TABLE 1

| Compound | | |
|---|---|---|
| ID | Name | Chemical Structure |
| 1002e | m⁷I(ppp) Am(p)G | 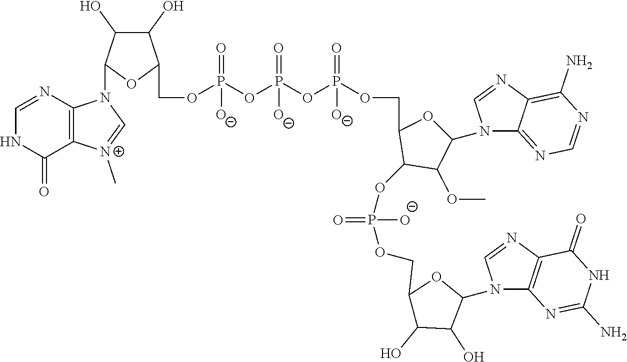 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]cnc43)C(O)C2O)OC1n1cnc2c(N)ncnc21] |
| 1002i | m⁷E(ppp) Am(p)G | 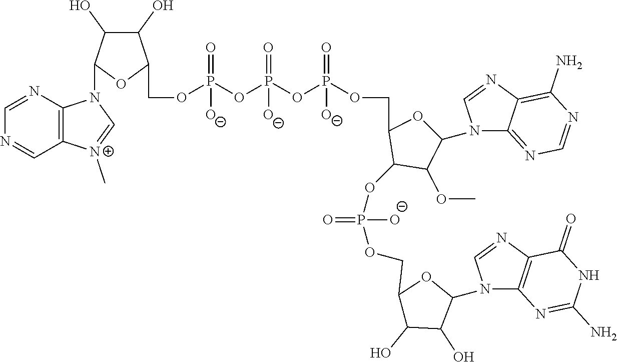 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4cncnc43)C(O)C2O)OC1n1cnc2c(N)ncnc21] |
| 5201 | m⁷I(ppp) Am(p)G(p)G | 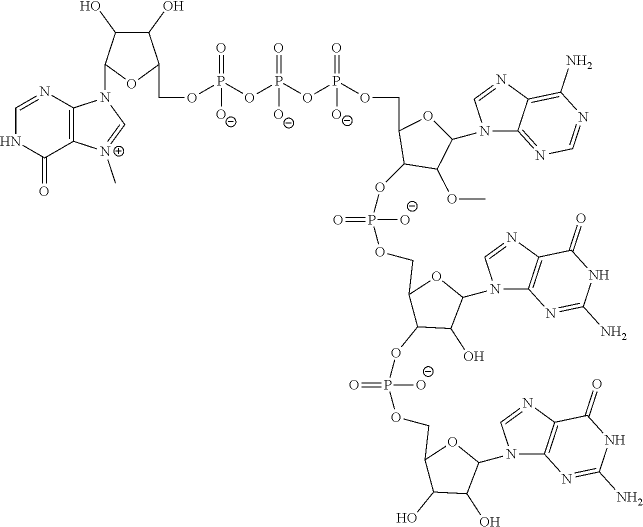 [SMILES: COC1C(OP(=O)([O-])OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]cnc43)C(O)C2O)OC1n1cnc2c(N)ncnc21] |
| 5202 | m⁷E(ppp) Am(p)G(p)G | 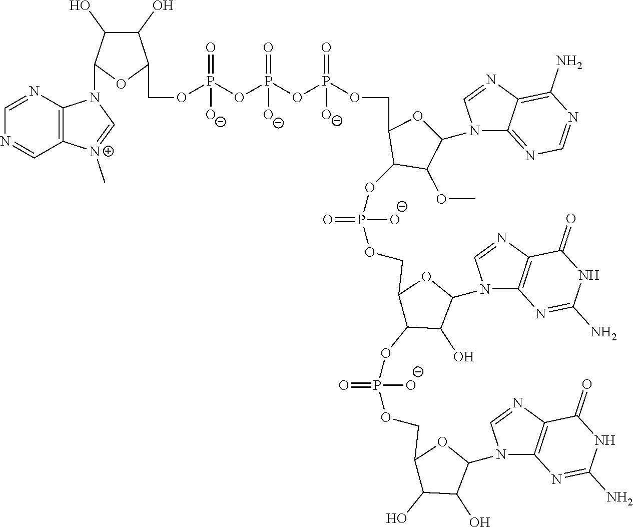 [SMILES: COC1C(OP(=O)([O-])OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4cncnc43)C(O)C2O)OC1n1cnc2c(N)ncnc21] |
| 1007a | m⁷G(ppp) Am(ps)G | 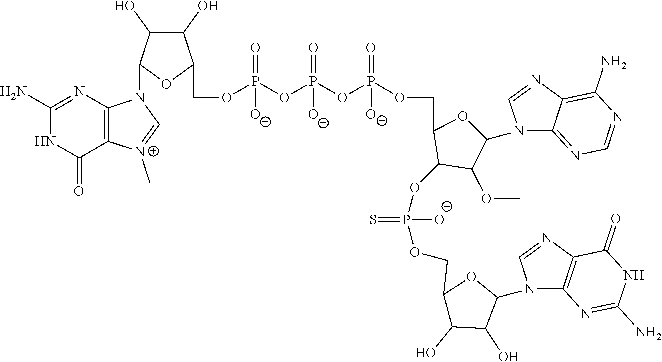 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |
| 1007a_D1 | m⁷G(ppp) Am(ps)G | 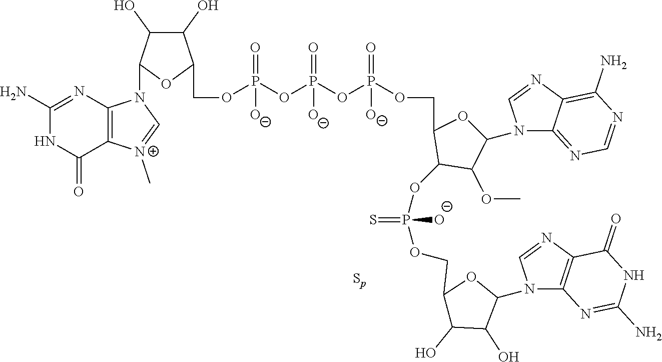 [SMILES: COC1C(O[P@@]([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |
| 1007a_D2 | m⁷G(ppp) Am(ps)G | 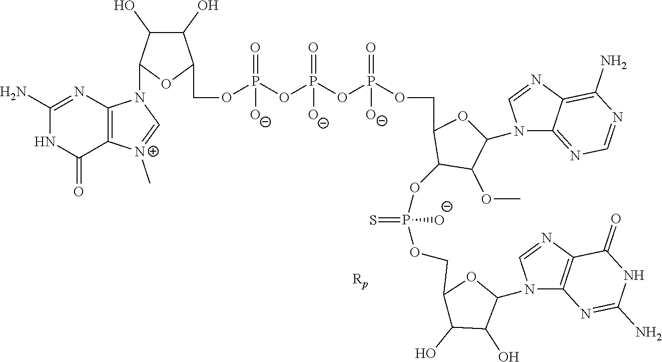 [SMILES: COC1C(O[P@]([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |
| 5203 | m⁷G(ppp) Am(ps)G(p)G | 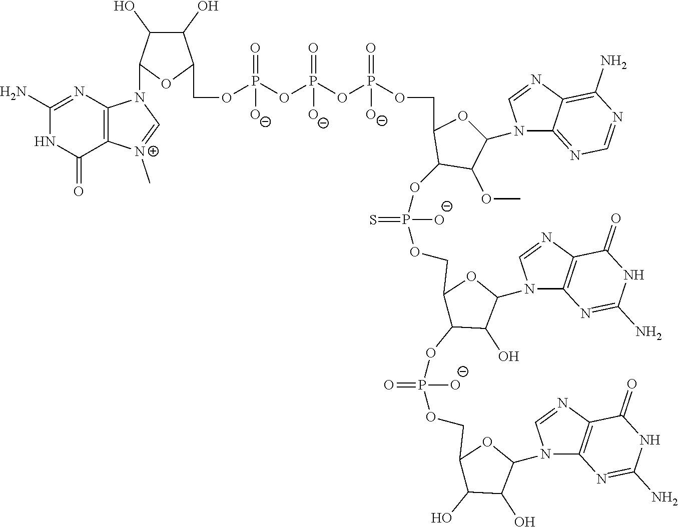 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |
| 5204 | m⁷G(ppp) Am(ps)G(ps) | 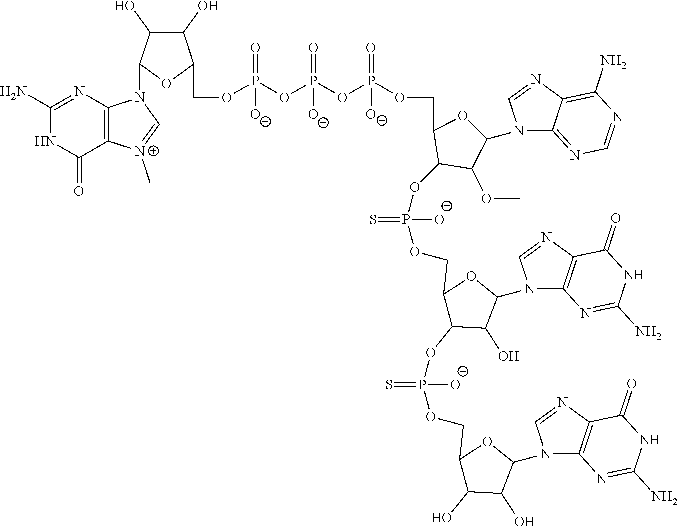 [SMILES: COC1C(OP([O-])(=S)OCC2OC(n3cnc4c(=O)[nH]c(N)nc43)C(O)C2OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |
| 1007e | m⁷I(ppp) Am(ps)G | 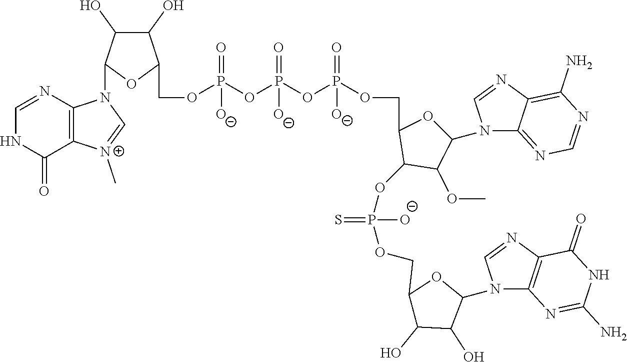 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]cnc43)C(O)C2O)OC1n1cnc2c(N)ncnc21] |
| 5205 | m⁷I(ppp) Am(ps)G(p)G | 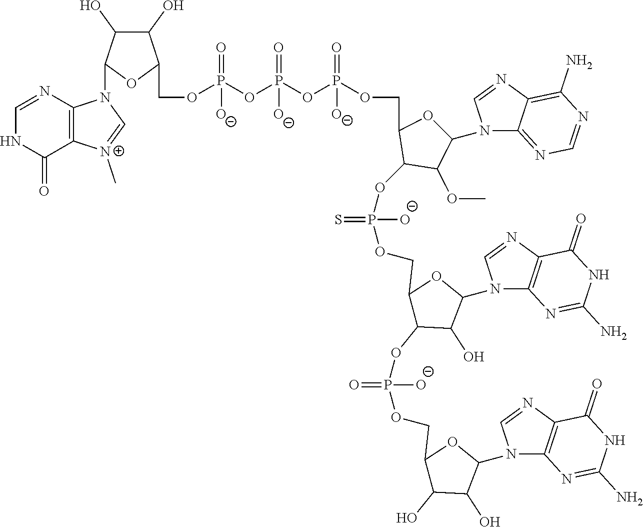 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]cnc43)C(O)C2O)OC1n1cnc2c(N)ncnc21] |
| 1007i | m⁷E(ppp) Am(ps)G | 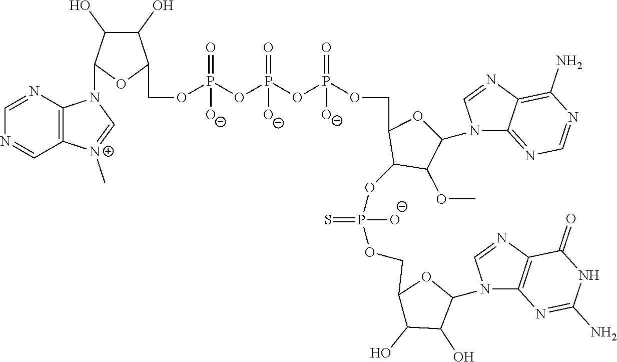 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4cncnc43)C(O)C2O)OC1n1cnc2c(N)ncnc21] |
| 5206 | m⁷E(ppp) Am(ps)G(p)G | 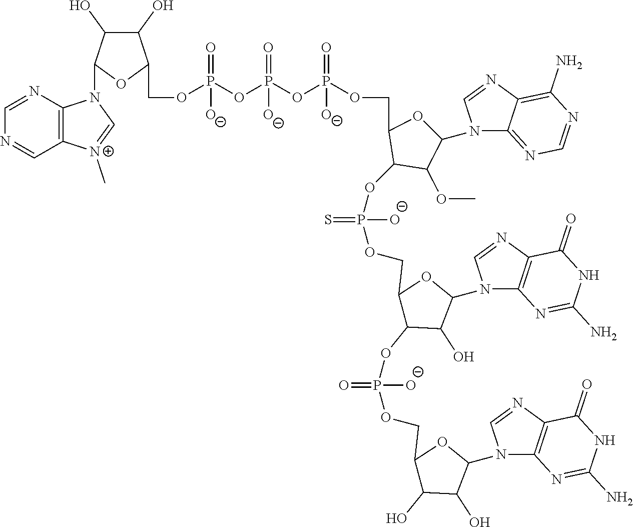 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4cncnc43)C(O)C2O)OC1n1cnc2c(N)ncnc21] |
| 1032a | m⁷G(ppsps) Am(p)G | 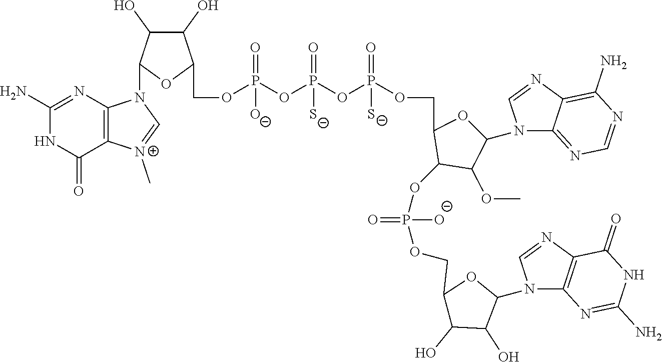 [SMILES: COC1C(OP(=O)([O-])OCC2OC(n3cnc4c(=O)[nH]c(N)nc43)C(O)C2O)C(COP(=O)([S-])OP(=O)([S-])OP(=O)([O-])OCC2OC(n3c[n+](C)c4c(=O)[nH]c(N)nc43)C(O)C2O)OC1n1cnc2c(N)ncnc21] |
| 1037a | m⁷G(ppsps) Am(ps)G | 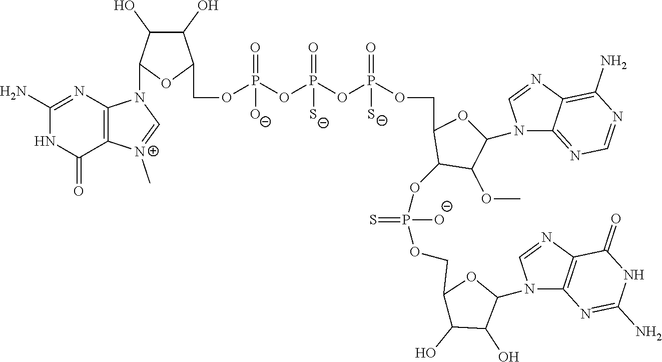 [SMILES: COC1C(OP([O-])(=S)OCC2OC(n3cnc4c(=O)[nH]c(N)nc43)C(O)C2O)C(COP(=O)([S-])OP(=O)([S-])OP(=O)([O-])OCC2OC(n3c[n+](C)c4c(=O)[nH]c(N)nc43)C(O)C2O)OC1n1cnc2c(N)ncnc21] |
| 1107a | m⁷G(ppsp) Am(p)G | 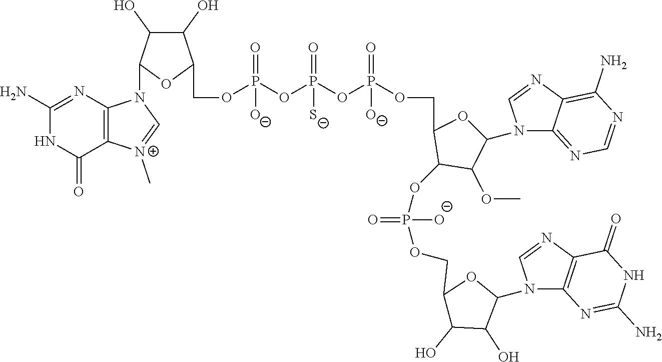 [SMILES: COC1C(OP(=O)([O-])OCC2OC(n3cnc4c(=O)[nH]c(N)nc43)C(O)C2O)C(COP(=O)([O-])OP(=O)([S-])OP(=O)([O-])OCC2OC(n3c[n+](C)c4c(=O)[nH]c(N)nc43)C(O)C2O)OC1n1cnc2c(N)ncnc21] |
| 5207 | m⁷G(ppsp) Am(p)G(p)G | 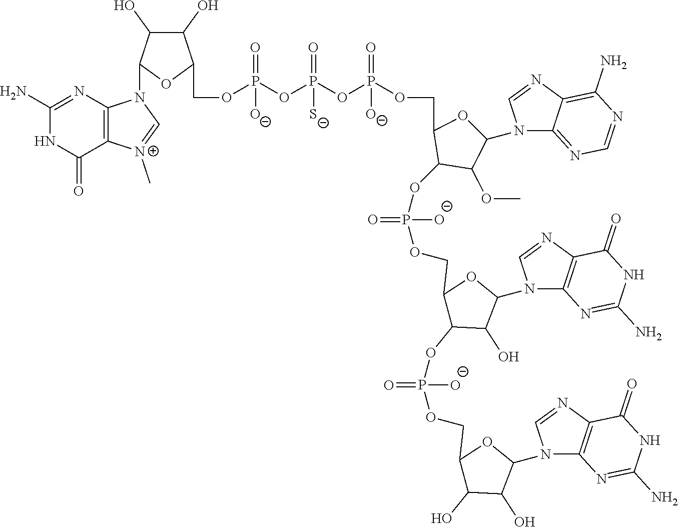 [SMILES: COC1C(OP(=O)([O-])OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([S-])OP(=O)([O-])OCC2OC(n3c[n+](C)c4c(=O)[nH]c(N)nc43)C(O)C2O)OC1n1cnc2c(N)ncnc21] |
| 1112a | m⁷G(ppsp) Am(ps)G | 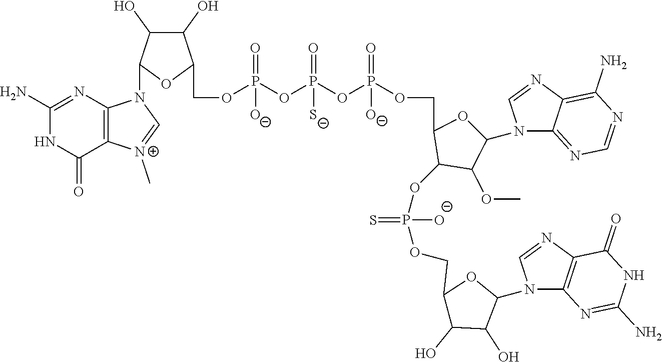 [SMILES: COC1C(OP([O-])(=S)OCC2OC(n3cnc4c(=O)[nH]c(N)nc43)C(O)C2O)C(COP(=O)([O-])OP(=O)([S-])OP(=O)([O-])OCC2OC(n3c[n+](C)c4c(=O)[nH]c(N)nc43)C(O)C2O)OC1n1cnc2c(N)ncnc21] |
| 1137a | m⁷G(ppssp) Am(p)G | 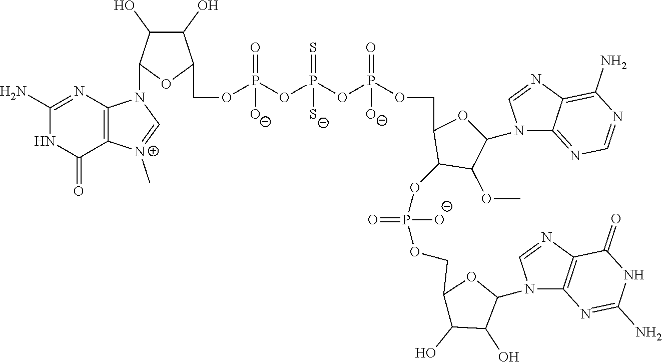 [SMILES: COC1C(OP(=O)([O-])OCC2OC(n3cnc4c(=O)[nH]c(N)nc43)C(O)C2O)C(COP(=O)([O-])OP(=S)([S-])OP(=O)([O-])OCC2OC(n3c[n+](C)c4c(=O)[nH]c(N)nc43)C(O)C2O)OC1n1cnc2c(N)ncnc21] |
| 1141a | m⁷G(ppssp) Am(ps)G | 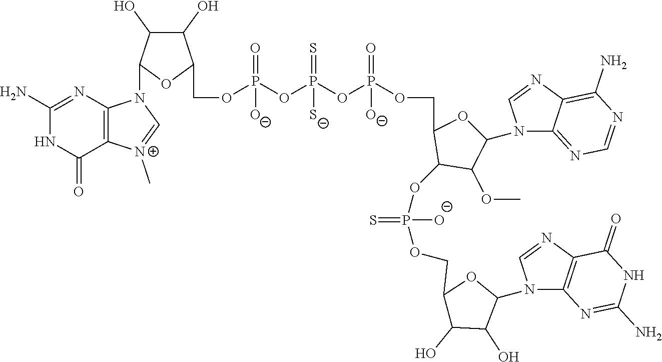 [SMILES: COC1C(OP([O-])(=S)OCC2OC(n3cnc4c(=O)[nH]c(N)nc43)C(O)C2O)C(COP(=O)([O-])OP(=S)([S-])OP(=O)([O-])OCC2OC(n3c[n+](C)c4c(=O)[nH]c(N)nc43)C(O)C2O)OC1n1cnc2c(N)ncnc21] |
| 5208 | m⁷G(ppssp) Am(p)G(p) | 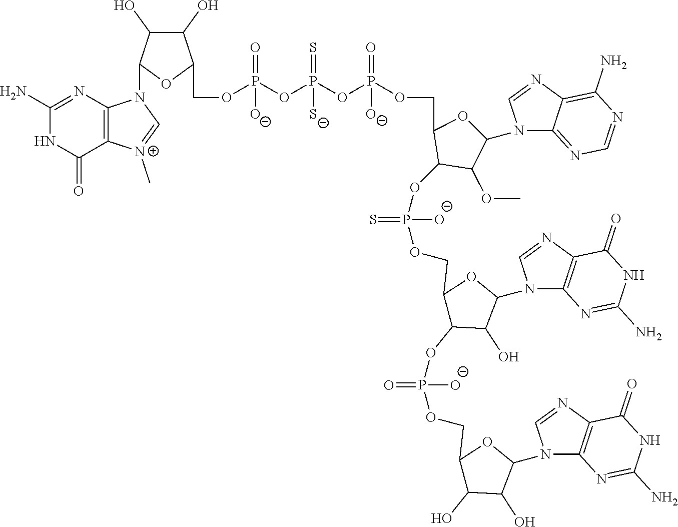 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=S)([S-])OP(=O)([O-])OCC2OC(n3c[n+](C)c4c(=O)[nH]c(N)nc43)C(O)C2O)OC1n1cnc2c(N)ncnc21] |
| 3102a | m⁷G(ppp) Em(p)G | 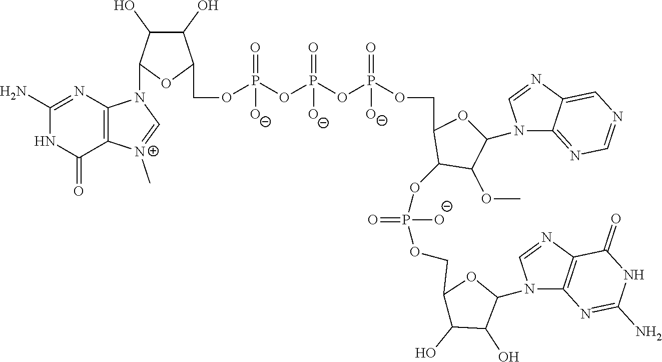 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2cncnc21] |
| 3107a | m⁷G(ppp) Em(ps)G | 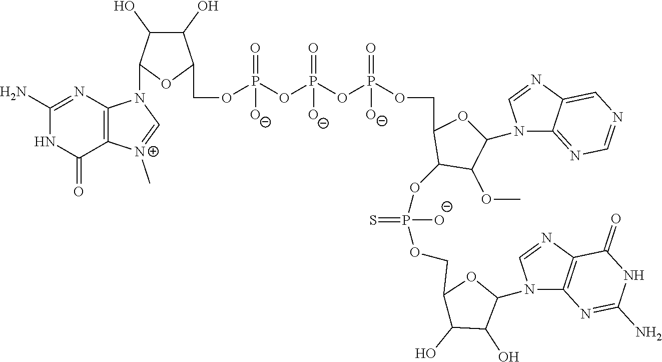 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2cncnc21] |
| 3207a | m1G(ppsp) Em(p)G | 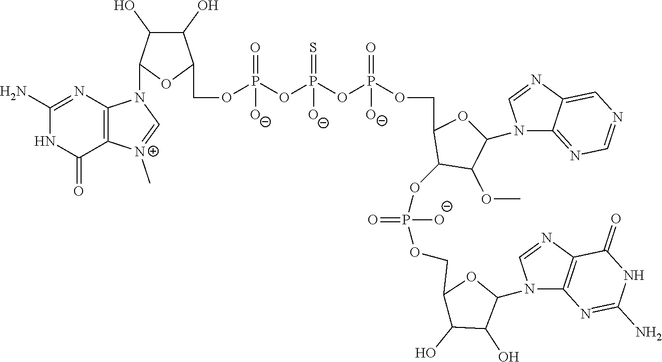 [SMILES: COC1C(OP(=O)([O-])OCC2OC(n3cnc4c(=O)[nH]c(N)nc43)C(O)C2O)C(COP(=O)([O-])OP([O-])(=S)OP(=O)([O-])OCC2OC(n3c[n+](C)c4c(=O)[nH]c(N)nc43)C(O)C2O)OC1n1cnc2cncnc21] |
| 3212a | m⁷G(ppsp) Em(ps)G | 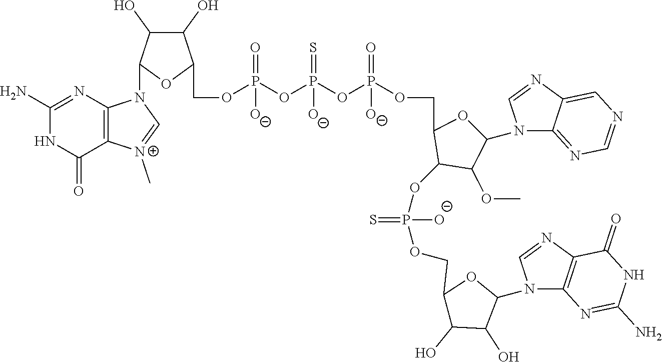 [SMILES: COC1C(OP([O-])(=S)OCC2OC(n3cnc4c(=O)[nH]c(N)nc43)C(O)C2O)C(COP(=O)([O-])OP([O-])(=S)OP(=O)([O-])OCC2OC(n3c[n+](C)c4c(=O)[nH]c(N)nc43)C(O)C2O)OC1n1cnc2cncnc21] |
| 5209 | m⁷G(ppp) Em(ps)G(p)G | 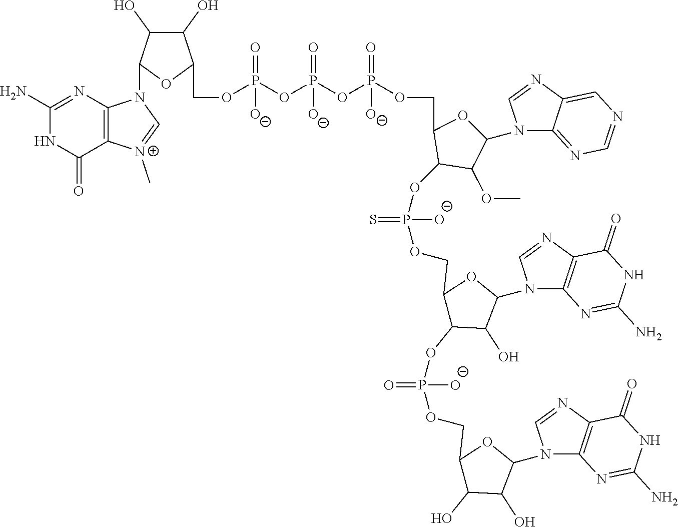 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2cncnc21] |
| 1007c | m⁷G(3′OMe)(ppp) Am(ps)G | 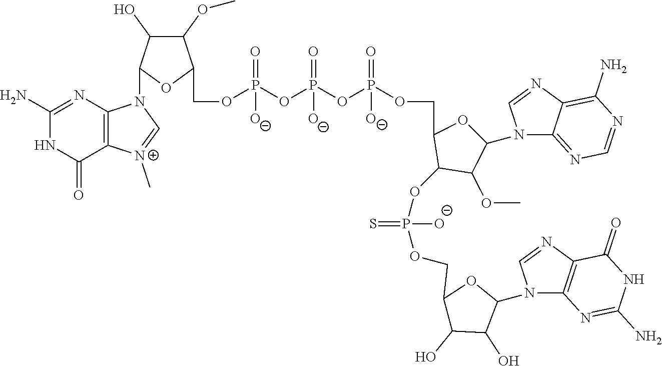 [SMILES: COC1C(COP(=O)([O-])OP(=O)([O-])OP(=O)([O-])OCC2OC(n3cnc4c(N)ncnc43)C(OC)C2OP([O-])(=S)OCC2OC(n3cnc4c(=O)[nH]c(N)nc43)C(O)C2O)OC(n2c[n+](C)c3c(=O)[nH]c(N)nc32)C1O] |
| 1107c | m⁷G(3′OMe) (ppsp)Am(p)G | 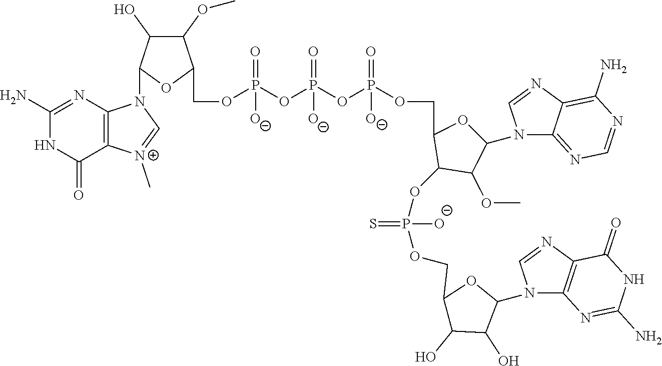 [SMILES: COC1C(COP(=O)([O-])OP(=O)([O-])OP(=O)([O-])OCC2OC(n3cnc4c(N)ncnc43)C(OC)C2OP([O-])(=S)OCC2OC(n3cnc4c(=O)[nH]c(N)nc43)C(O)C2O)OC(n2c[n+](C)c3c(=O)[nH]c(N)nc32)C1O] |
| 1112c | m⁷G(3′OMe) (ppsp)Am(ps)G | 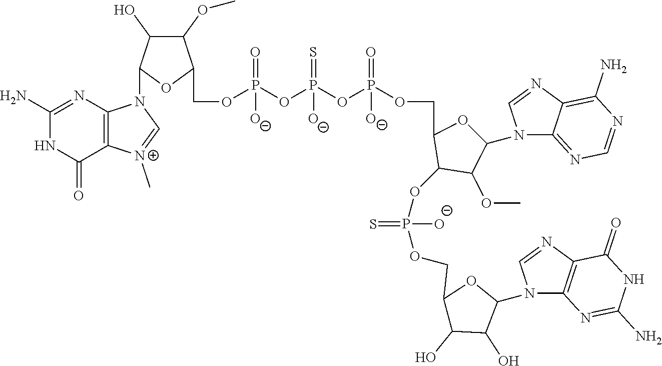 [SMILES: COC1C(COP(=O)([O-])OP([O-])(=S)OP(=O)([O-])OCC2OC(n3cnc4c(N)ncnc43)C(OC)C2OP([O-])(=S)OCC2OC(n3cnc4c(=O)[nH]c(N)nc43)C(O)C2O)OC(n2c[n+](C)c3c(=O)[nH]c(N)nc32)C1O] |
| 5210 | m⁷G(3′OMe)(ppp) Am (ps)G(p)G | 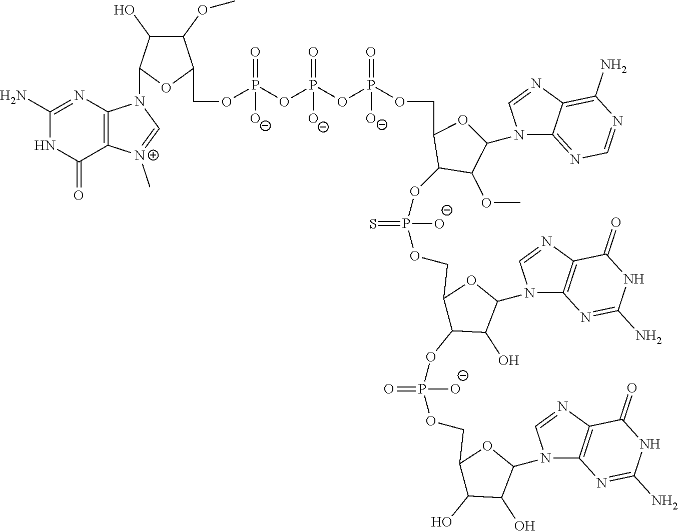 [SMILES: COC1C(COP(=O)([O-])OP(=O)([O-])OP(=O)([O-])OCC2OC(n3cnc4c(N)ncnc43)C(OC)C2OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)OC(n2c[n+](C)c3c(=O)[nH]c(N)nc32)C1O] |
| 3102c | m⁷G(3′OMe) (ppp)Em (p)G | 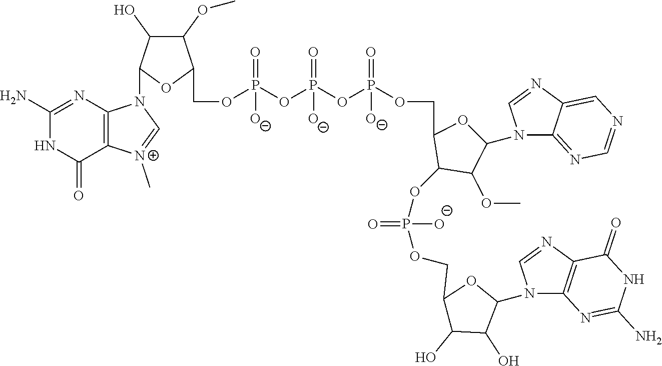 [SMILES: COC1C(COP(=O)([O-])OP(=O)([O-])OP(=O)([O-])OCC2OC(n3cnc4cncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O] |
| 3107c | m⁷G(3′OMe) (ppp)Em(ps)G | 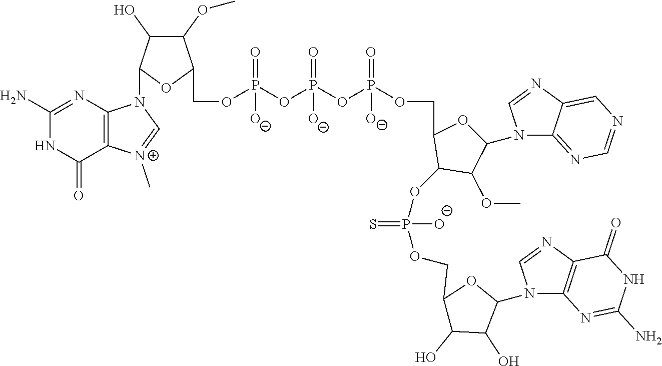 [SMILES: COC1C(COP(=O)([O-])OP(=O)([O-])OP(=O)([O-])OCC2OC(n3cnc4cncnc43)C(OC)C2OP([O-])(=S)OCC2OC(n3cnc4c(=O)[nH]c(N)nc43)C(O)C2O)OC(n2c[n+](C)c3c(=O)[nH]c(N)nc32)C1O] |
| 3207c | m⁷G(3′OMe) (ppsp)Em (p)G | 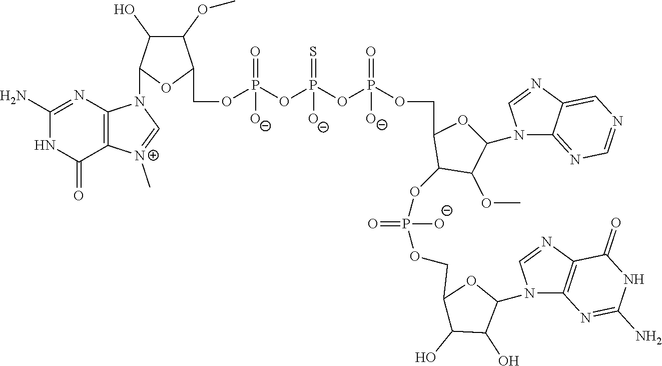 [SMILES: COC1C(COP(=O)([O-])OP([O-])(=S)OP(=O)([O-])OCC2OC(n3cnc4cncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O] |
| 3212c | m⁷G(3′OMe) (ppsp) Em(ps)G | 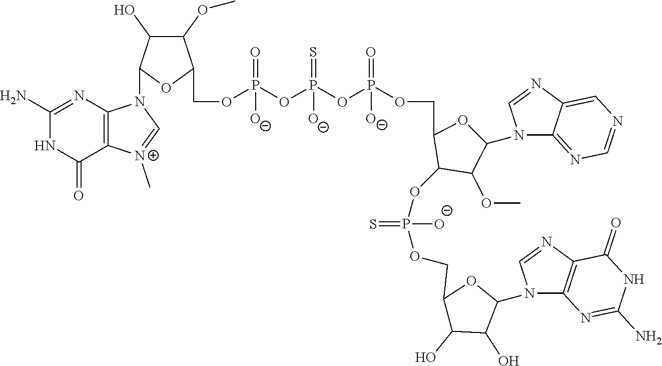 [SMILES: COC1C(COP(=O)([O-])OP([O-])(=S)OP(=O)([O-])OCC2OC(n3cnc4cncnc43)C(OC)C2OP([O-])(=S)OCC2OC(n3cnc4c(=O)[nH]c(N)nc43)C(O)C2O)OC(n2c[n+](C)c3c(=O)[nH]c(N)nc32)C1O] |
| 5211 | m⁷G(3′OMe) (ppp)Em (ps)G(p)G | 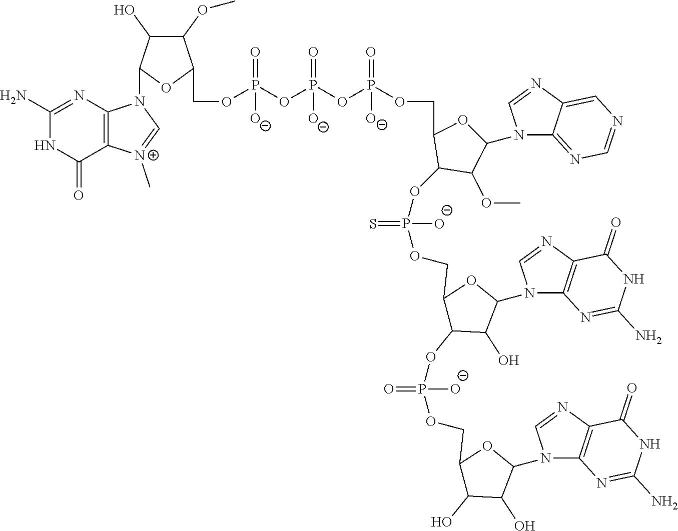 [SMILES: COC1C(COP(=O)([O-])OP(=O)([O-])OP(=O)([O-])OCC2OC(n3cnc4cncnc43)C(OC)C2OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)OC(n2c[n+](C)c3c(=O)[nH]c(N)nc32)C1O] |
| 5212 | m⁷G(ppp) Am(p)G(4- thio) | 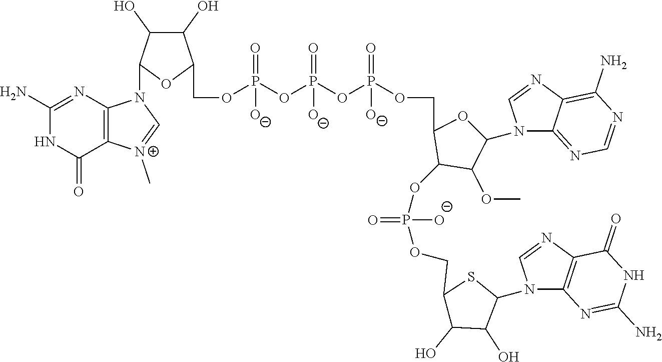 [SMILES: COC1C(OP(=O)([O-])OCC2SC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |
| 5213 | m⁷G(ppp) Am(p)G(4- thio)(p)G | 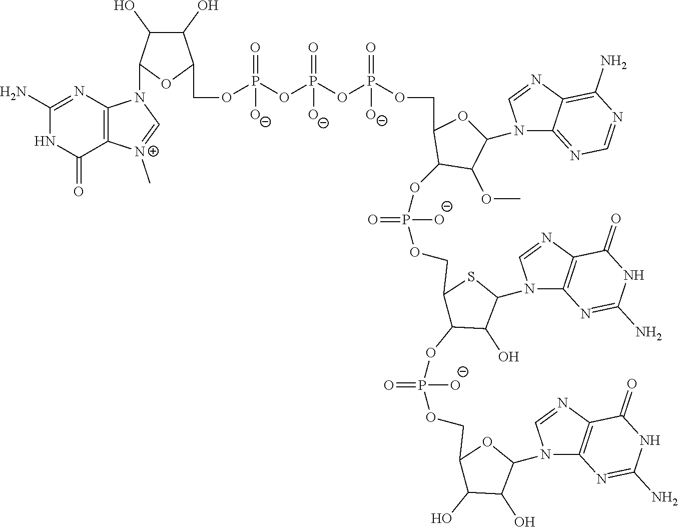 [SMILES: COC1C(OP(=O)([O-])OCC2SC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |
| 5214 | m⁷G(ppp) Am(ps)G(4- thio)(p)G | 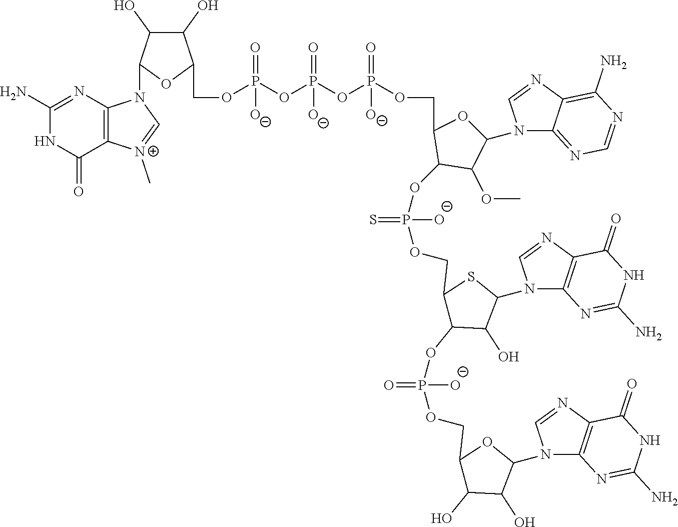 [SMILES: COC1C(OP([O-])(=S)OCC2SC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |

In some embodiments, disclosed herein is a pharmaceutically acceptable salt or pharmaceutically acceptable solvate or a pharmaceutical composition comprising one or more mRNA(s) which is/are produced/manufactured from one or more sequence initiator compound selected from Table 1, wherein the mRNA encode(s) one or more pharmaceutically active protein(s).

In certain embodiments the sequence initiator compound is as described in Table 2.

TABLE 2

| | | |
|---|---|---|
| 5215 | m⁷G(2′F- 2′F)(ppp) Am(p)G | 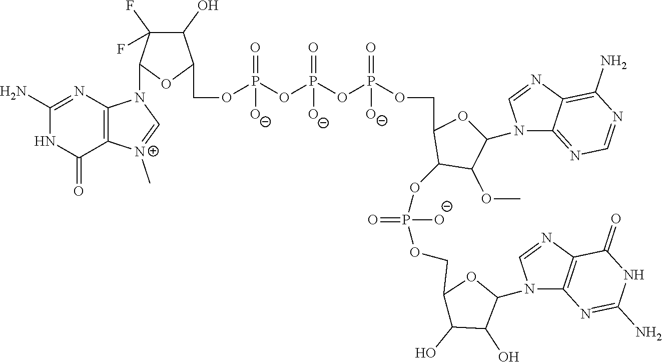 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(F)(F)C2O)OC1n1cnc2c(N)ncnc21] |
| 5216 | m⁷G(2′F- 2′F)(ppp) Am(ps)G | 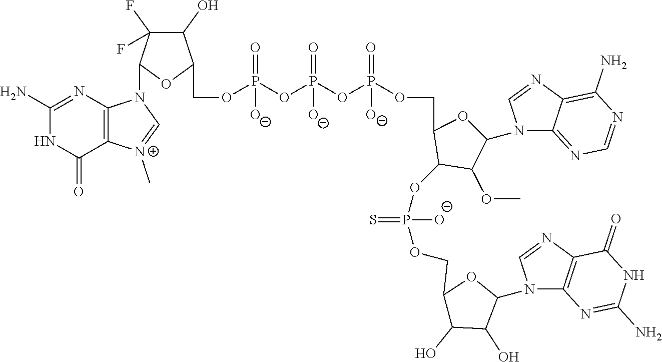 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(F)(F)C2O)OC1n1cnc2c(N)ncnc21] |
| 5217 | m⁷G(2′F- 2′F)(ppp) Am(p)G(p)G | 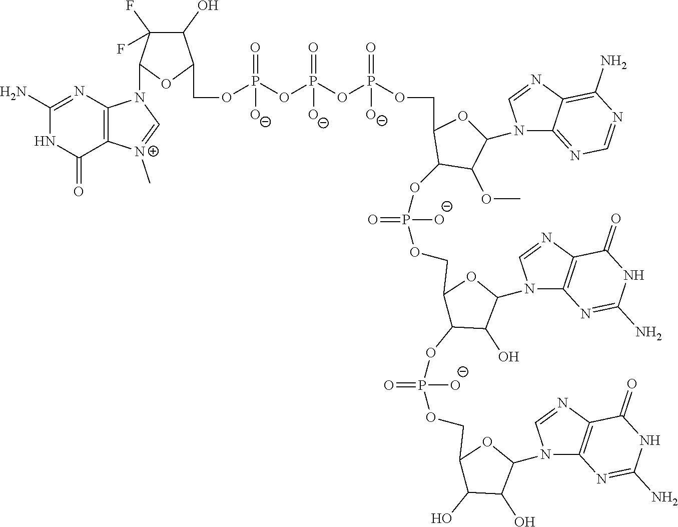 [SMILES: COC1C(OP(=O)([O-])OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(F)(F)C2O)OC1n1cnc2c(N)ncnc21] |
| 5218 | m⁷G(2′F- 2′F)(ppp) Am(ps)G(p)G | 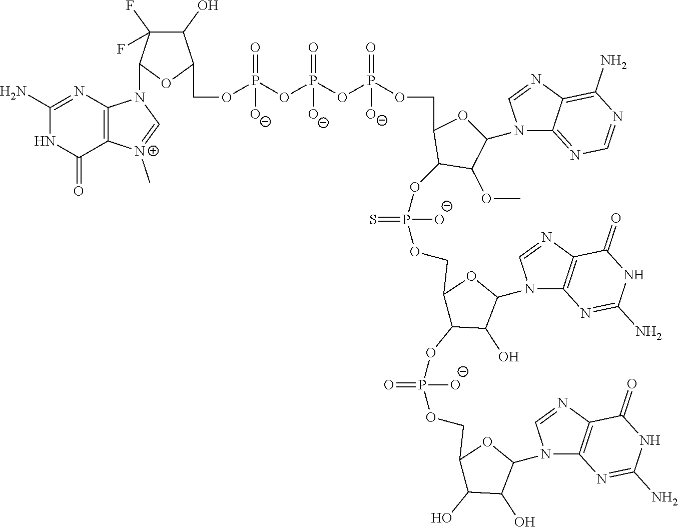 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(F)(F)C2O)OC1n1cnc2c(N)ncnc21] |
| 5219 | m⁷G(2′F- 2′Me)(ppp) Am(p)G | 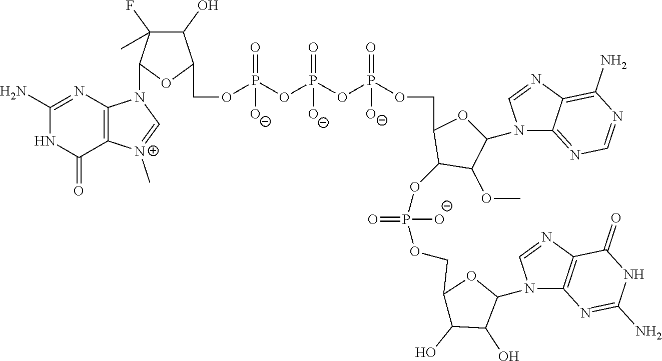 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(C)(F)C2O)OC1n1cnc2c(N)ncnc21] |
| 5220 | m⁷G(2′F- 2′Me)(ppp) Am(ps)G | 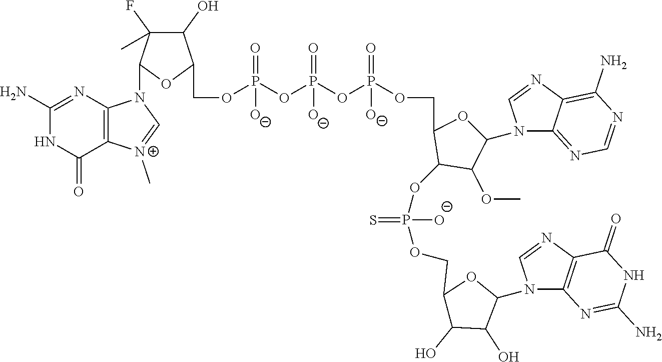 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(C)(F)C2O)OC1n1cnc2c(N)ncnc21] |
| 5221 | m⁷G(2′F- 2′Me)(ppp) Am(p)G(p)G | 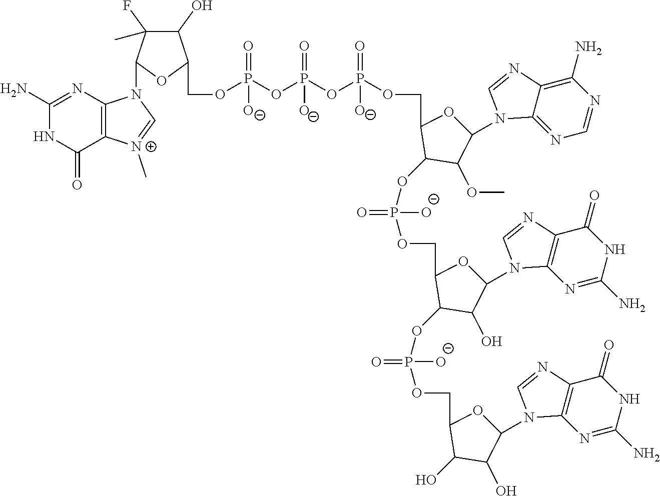 [SMILES: COC1C(OP(=O)([O-])OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(C)(F)C2O)OC1n1cnc2c(N)ncnc21] |
| 5222 | m⁷G(2′F- 2′Me)(ppp) Am(ps)G(p)G | 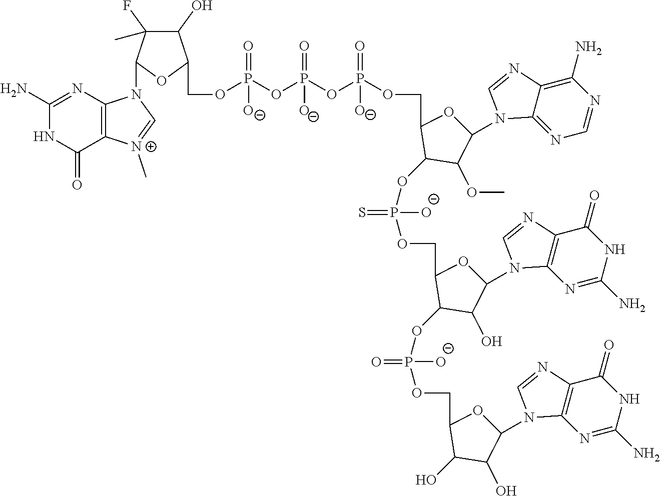 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(C)(F)C2O)OC1n1cnc2c(N)ncnc21] |
| 5223 | m⁷G(2′Me- 2′OH)(ppp) Am(p)G | 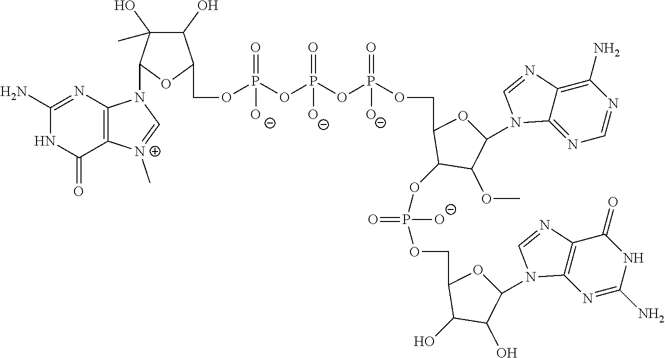 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(C)(O)C2O)OC1n1cnc2c(N)ncnc21] |
| 5224 | m⁷G(2′Me- 2′OH)(ppp) Am(ps)G | 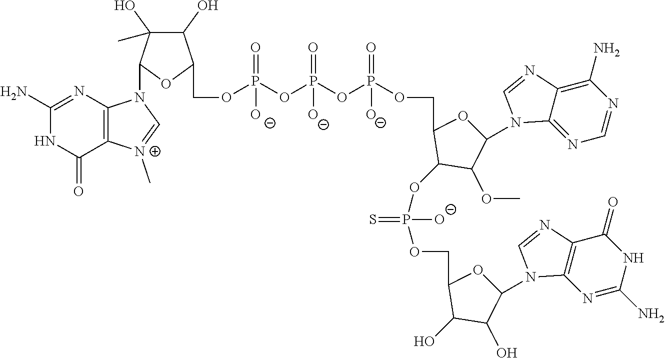 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(C)(O)C2O)OC1n1cnc2c(N)ncnc21] |
| 5225 | m⁷G(2′Me- 2′OH)(ppp) Am(p)G(p) G | 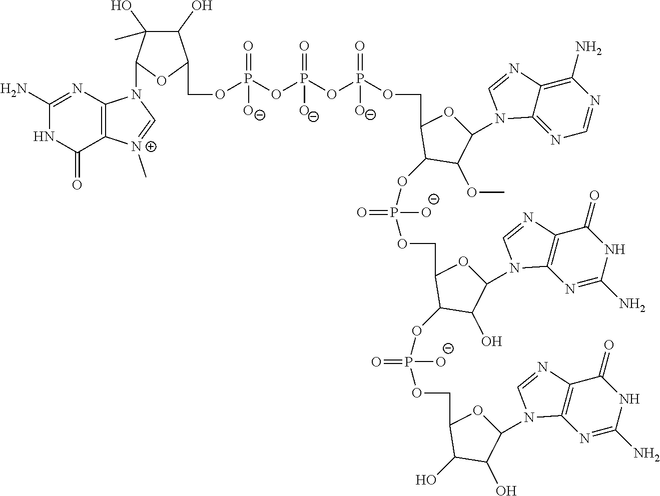 [SMILES: COC1C(OP(=O)([O-])OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(C)(O)C2O)OC1n1cnc2c(N)ncnc21] |
| 5226 | m⁷G(2′Me- 2′OH)(ppp) Am(ps)G(p) G | 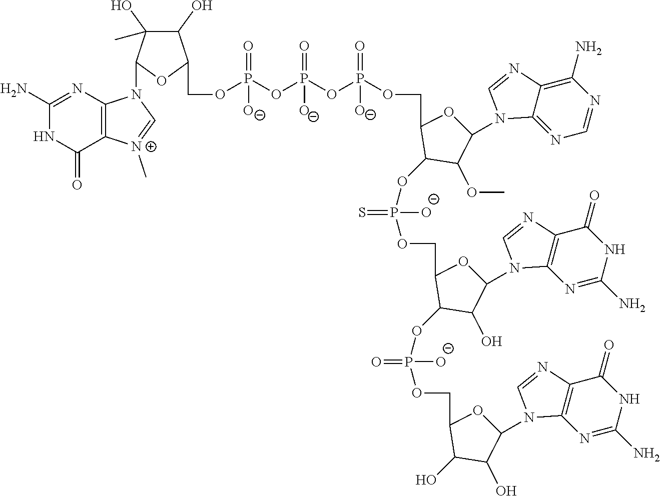 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(C)(O)C2O)OC1n1cnc2c(N)ncnc21] |
| 5227 | m⁷G(4′OMe)(ppp) Am(p)G | 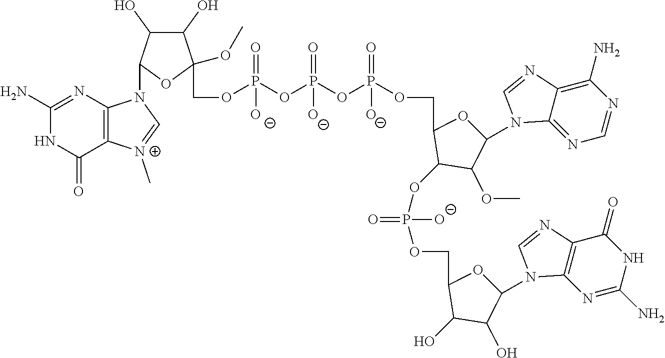 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2(OC)OC(n3c[n+](C)c4c(=O)[nH]c(N)nc43)C(O)C2O)OC1n1cnc2c(N)ncnc21] |
| 5228 | m⁷G(4′OMe)(ppp) Am(ps)G | 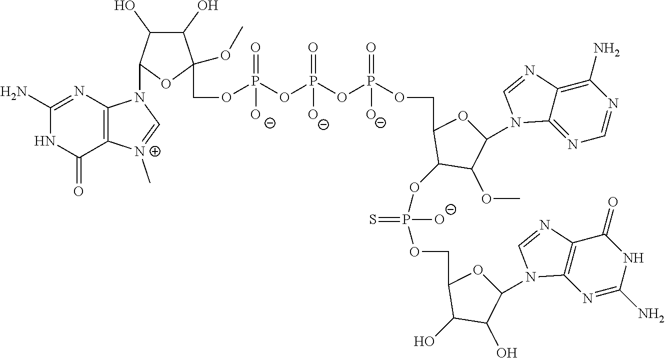 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2(OC)OC(n3c[n+](C)c4c(=O)[nH]c(N)nc43)C(O)C2O)OC1n1cnc2c(N)ncnc21] |
| 5229 | m⁷G(4′OMe)(ppp) Am(p)G(p)G | 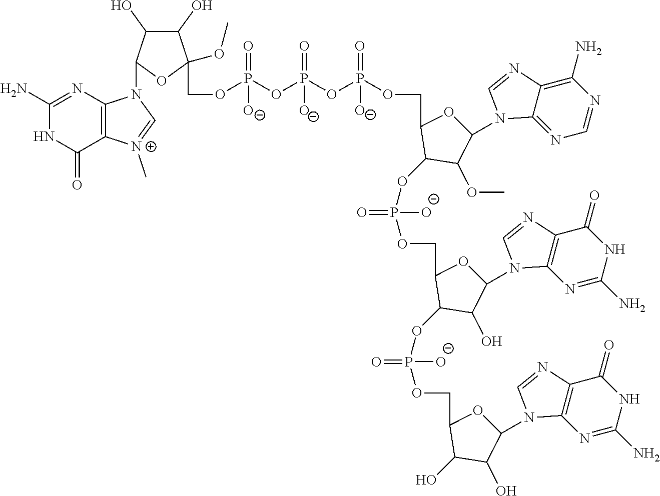 [SMILES: COC1C(OP(=O)([O-])OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2(OC)OC(n3c[n+](C)c4c(=O)[nH]c(N)nc43)C(O)C2O)OC1n1cnc2c(N)ncnc21] |
| 5230 | m⁷G(4′OMe)(ppp) Am(ps)G(p)G | 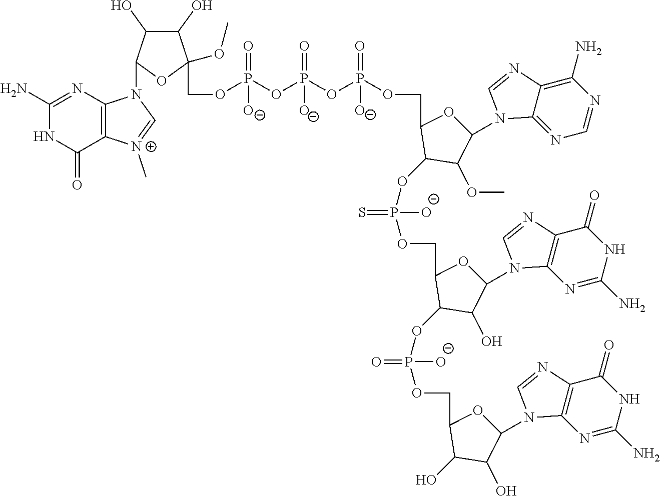 [SMILES: COC1C(OP([O-])(=S)OCC2OC(n3cnc4c(=O)[nH]c(N)nc43)C(O)C2OP(=O)([O-])OCC2OC(n3cnc4c(=O)[nH]c(N)nc43)C(O)C2O)C(COP(=O)([O-])OP(=O)([O-])OP(=O)([O-])OCC2(OC)OC(n3c[n+](C)c4c(=O)[nH]c(N)nc43)C(O)C2O)OC1n1cnc2c(N)ncnc21] |
| 5231 | 4-Cl- Bn7G(ppp) Am(p)G | 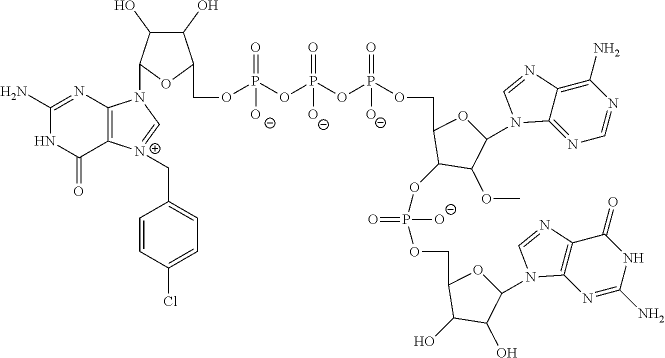 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](Cc4ccc(Cl)cc4)c4c(=O)[nH]c(N)nc43)C(O)C2O)OC1n1cnc2c(N)ncnc21] |
| 5232 | m⁷G(ppp)2,6- DAPm(p)G | 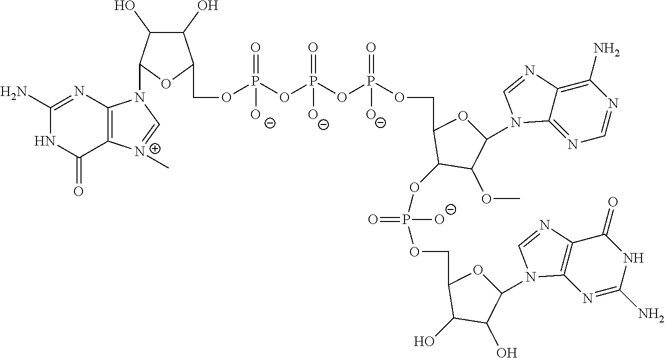 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21] |
| 5233 | 4-Cl-Bn7G(ppp)2,6- DAPm(p)G | 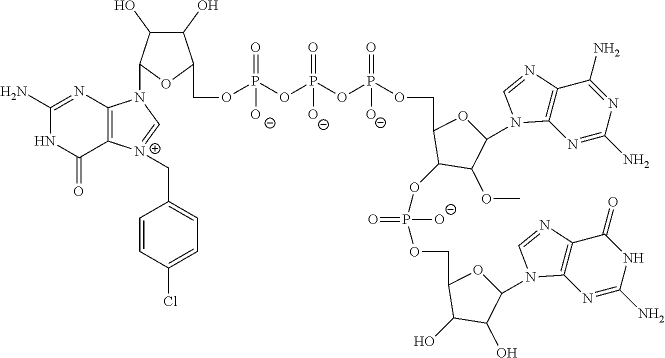 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](Cc4ccc(Cl)cc4)c4c(=O)[nH]c(N)nc43)C(O)C2O)OC1n1cnc2c(N)nc(N)nc21] |
| 5234 | 4-Cl- Bn7G(ppp) Am(ps)G | 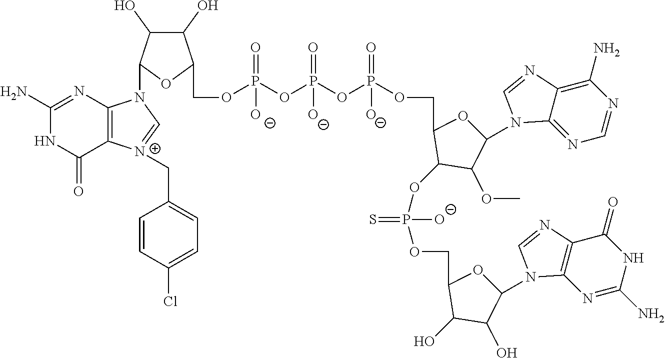 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](Cc4ccc(Cl)cc4)c4c(=O)[nH]c(N)nc43)C(O)C2O)OC1n1cnc2c(N)ncnc21] |
| 5235 | m⁷G(ppp)2,6- DAPm(ps)G | 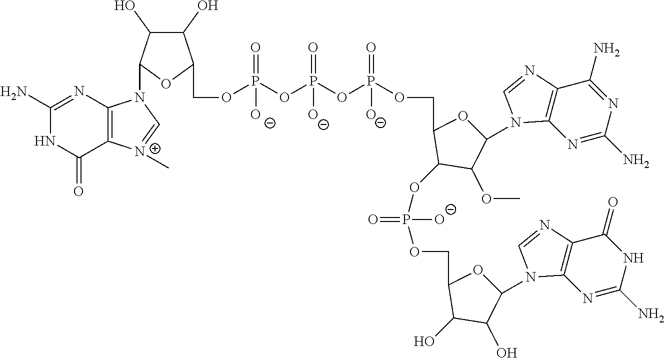 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)nc(N)nc21] |
| 5236 | 4-Cl-Bn7G(ppp)2,6- DAPm(ps)G | 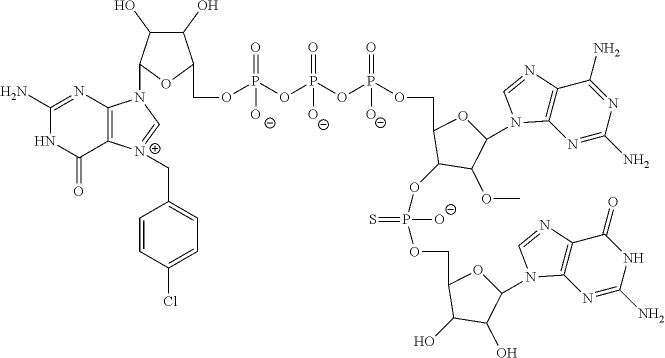 [SMILES: COC1C(OP([O-])(=S)OCC2OC(n3cnc4c(=O)[nH]c(N)nc43)C(O)C2O)C(COP(=O)([O-])OP(=O)([O-])OP(=O)([O-])OCC2OC(n3c[n+](Cc4ccc(Cl)cc4)c4c(=O)[nH]c(N)nc43)C(O)C2O)OC1n1cnc2c(N)nc(N)nc21] |

In some embodiments, disclosed herein is a pharmaceutically acceptable salt or pharmaceutically acceptable solvate or a pharmaceutical composition comprising one or more mRNA(s) which is/are produced/manufactured from one or more sequence initiator compound selected from Table 2, wherein the mRNA encode(s) one or more pharmaceutically active protein(s).

In certain embodiments the sequence initiator compound is as described in Table 3.

TABLE 3
CapA Structures for mRNA preparation

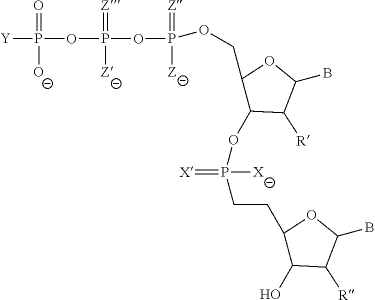

| Compound | Sequence | X | X′ | Z | Z′ | Z″ | Z′′′ | R′ | R″ |
|---|---|---|---|---|---|---|---|---|---|
| 1001a-l | Y*AG | O | O | O | O | O | O | OH | OH |
| 1002a-l | Y*AG | O | O | O | O | O | O | OMe | OH |
| 1003a-l | Y*AG | O | O | O | O | O | O | H | OH |
| 1004a-l | Y*AG | O | O | O | O | O | O | F | OH |
| 1005a-l | Y*AG | O | O | O | O | O | O | OEt | OH |
| 1006a-l | Y*AG | S | O | O | O | O | O | OH | OH |
| 1007a-l | Y*AG | S | O | O | O | O | O | OMe | OH |
| 1008a-l | Y*AG | S | O | O | O | O | O | H | OH |
| 1009a-l | Y*AG | S | O | O | O | O | O | F | OH |
| 1010a-l | Y*AG | S | O | O | O | O | O | OEt | OH |
| 1011a-l | Y*AG | S | S | O | O | O | O | OH | OH |
| 1012a-l | Y*AG | S | S | O | O | O | O | OMe | OH |
| 1013a-l | Y*AG | S | S | O | O | O | O | H | OH |
| 1014a-l | Y*AG | S | S | O | O | O | O | F | OH |
| 1015a-l | Y*AG | S | S | O | O | O | O | OEt | OH |
| 1016a-l | Y*AG | O | O | S | O | O | O | OH | OH |
| 1017a-l | Y*AG | O | O | S | O | O | O | OMe | OH |
| 1018a-l | Y*AG | O | O | S | O | O | O | H | OH |
| 1019a-l | Y*AG | O | O | S | O | O | O | F | OH |
| 1020a-l | Y*AG | O | O | S | O | O | O | OEt | OH |
| 1021a-l | Y*AG | S | O | S | O | O | O | OH | OH |
| 1022a-l | Y*AG | S | O | S | O | O | O | OMe | OH |
| 1023a-l | Y*AG | S | O | S | O | O | O | H | OH |
| 1024a-l | Y*AG | S | O | S | O | O | O | F | OH |
| 1025a-l | Y*AG | S | O | S | O | O | O | OEt | OH |
| 1026a-l | Y*AG | S | S | S | O | O | O | OH | OH |
| 1027a-l | Y*AG | S | S | S | O | O | O | OMe | OH |
| 1028a-l | Y*AG | S | S | S | O | O | O | H | OH |
| 1029a-l | Y*AG | S | S | S | O | O | O | F | OH |
| 1030a-l | Y*AG | S | S | S | O | O | O | OEt | OH |
| 1031a-l | Y*AG | O | O | S | S | O | O | OH | OH |
| 1032a-l | Y*AG | O | O | S | S | O | O | OMe | OH |
| 1033a-l | Y*AG | O | O | S | S | O | O | H | OH |
| 1034a-l | Y*AG | O | O | S | S | O | O | F | OH |
| 1035a-l | Y*AG | O | O | S | S | O | O | OEt | OH |
| 1036a-l | Y*AG | S | O | S | S | O | O | OH | OH |
| 1037a-l | Y*AG | S | O | S | S | O | O | OMe | OH |
| 1038a-l | Y*AG | S | O | S | S | O | O | H | OH |
| 1039a-l | Y*AG | S | O | S | S | O | O | F | OH |
| 1040a-l | Y*AG | S | O | S | S | O | O | OEt | OH |
| 1041a-l | Y*AG | S | S | S | S | O | O | OH | OH |
| 1042a-l | Y*AG | S | S | S | S | O | O | OMe | OH |
| 1043a-l | Y*AG | S | S | S | S | O | O | H | OH |
| 1044a-l | Y*AG | S | S | S | S | O | O | F | OH |
| 1045a-l | Y*AG | S | S | S | S | O | O | OEt | OH |
| 1046a-l | Y*AG | O | O | S | S | S | O | OH | OH |
| 1047a-l | Y*AG | O | O | S | S | S | O | OMe | OH |
| 1048a-l | Y*AG | O | O | S | S | S | O | H | OH |
| 1049a-l | Y*AG | O | O | S | S | S | O | F | OH |
| 1050a-l | Y*AG | O | O | S | S | S | O | OEt | OH |
| 1051a-l | Y*AG | S | O | S | S | S | O | OH | OH |
| 1052a-l | Y*AG | S | O | S | S | S | O | OMe | OH |
| 1053a-l | Y*AG | S | O | S | S | S | O | H | OH |
| 1054a-l | Y*AG | S | O | S | S | S | O | F | OH |
| 1055a-l | Y*AG | S | O | S | S | S | O | OEt | OH |
| 1056a-l | Y*AG | S | S | S | S | S | O | OH | OH |
| 1057a-l | Y*AG | S | S | S | S | S | O | OMe | OH |
| 1058a-l | Y*AG | S | S | S | S | S | O | H | OH |
| 1059a-l | Y*AG | S | S | S | S | S | O | F | OH |
| 1060a-l | Y*AG | S | S | S | S | S | O | OEt | OH |
| 1061a-l | Y*AG | O | O | S | S | S | S | OH | OH |
| 1062a-l | Y*AG | O | O | S | S | S | S | OMe | OH |
| 1063a-l | Y*AG | O | O | S | S | S | S | H | OH |
| 1064a-l | Y*AG | O | O | S | S | S | S | F | OH |
| 1065a-l | Y*AG | O | O | S | S | S | S | OEt | OH |
| 1066a-l | Y*AG | S | O | S | S | S | S | OH | OH |
| 1067a-l | Y*AG | S | O | S | S | S | S | OMe | OH |
| 1068a-l | Y*AG | S | O | S | S | S | S | H | OH |
| 1069a-l | Y*AG | S | O | S | S | S | S | F | OH |
| 1070a-l | Y*AG | S | O | S | S | S | S | OEt | OH |
| 1071a-l | Y*AG | S | S | S | S | S | S | OH | OH |
| 1072a-l | Y*AG | S | S | S | S | S | S | OMe | OH |
| 1073a-l | Y*AG | S | S | S | S | S | S | H | OH |
| 1074a-l | Y*AG | S | S | S | S | S | S | F | OH |
| 1075a-l | Y*AG | S | S | S | S | S | S | OEt | OH |
| 1076a-l | Y*AG | O | O | O | S | S | S | OH | OH |
| 1077a-l | Y*AG | O | O | O | S | S | S | OMe | OH |
| 1078a-l | Y*AG | O | O | O | S | S | S | H | OH |
| 1079a-l | Y*AG | O | O | O | S | S | S | F | OH |
| 1080a-l | Y*AG | O | O | O | S | S | S | OEt | OH |
| 1081a-l | Y*AG | S | O | O | S | S | S | OH | OH |
| 1082a-l | Y*AG | S | O | O | S | S | S | OMe | OH |
| 1083a-l | Y*AG | S | O | O | S | S | S | H | OH |
| 1084a-l | Y*AG | S | O | O | S | S | S | F | OH |
| 1085a-l | Y*AG | S | O | O | S | S | S | OEt | OH |
| 1086a-l | Y*AG | S | S | O | S | S | S | OH | OH |
| 1087a-l | Y*AG | S | S | O | S | S | S | OMe | OF |
| 1088a-l | Y*AG | S | S | O | S | S | S | H | OH |
| 1089a-l | Y*AG | S | S | O | S | S | S | F | OH |
| 1090a-l | Y*AG | S | S | O | S | S | S | OEt | OH |
| 1091a-l | Y*AG | O | O | O | O | S | S | OH | OH |
| 1092a-l | Y*AG | O | O | O | O | S | S | OMe | OH |
| 1093a-l | Y*AG | O | O | O | O | S | S | H | OH |
| 1094a-l | Y*AG | O | O | O | O | S | S | F | OF |
| 1095a-l | Y*AG | O | O | O | O | S | S | OEt | OH |
| 1096a-l | Y*AG | S | O | O | O | S | S | OH | OH |
| 1097a-l | Y*AG | S | O | O | O | S | S | OMe | OH |
| 1098a-l | Y*AG | S | O | O | O | S | S | H | OH |
| 1099a-l | Y*AG | S | O | O | O | S | S | F | OH |
| 1100a-l | Y*AG | S | O | O | O | S | S | OEt | OH |
| 1101a-l | Y*AG | S | S | O | O | S | S | OH | OF |
| 1102a-l | Y*AG | S | S | O | O | S | S | OMe | OH |
| 1103a-l | Y*AG | S | S | O | O | S | S | H | OF |
| 1104a-l | Y*AG | S | S | O | O | S | S | F | OH |
| 1105a-l | Y*AG | S | S | O | O | S | S | OEt | OH |
| 1106a-l | Y*AG | O | O | O | O | O | S | OH | OH |
| 1107a-l | Y*AG | O | O | O | O | O | S | OMe | OH |
| 1108a-l | Y*AG | O | O | O | O | O | S | H | OH |
| 1109a-l | Y*AG | O | O | O | O | O | S | F | OH |
| 1110a-l | Y*AG | O | O | O | O | O | S | OEt | OH |
| 1111a-l | Y*AG | S | O | O | O | O | S | OH | OH |
| 1112a-l | Y*AG | S | O | O | O | O | S | OMe | OH |
| 1113a-l | Y*AG | S | O | O | O | O | S | H | OH |
| 1114a-l | Y*AG | S | O | O | O | O | S | F | OH |
| 1115a-l | Y*AG | S | O | O | O | O | S | OEt | OH |
| 1116a-l | Y*AG | S | S | O | O | O | S | OH | OH |
| 1117a-l | Y*AG | S | S | O | O | O | S | OMe | OH |
| 1118a-l | Y*AG | S | S | O | O | O | S | H | OH |
| 1119a-l | Y*AG | S | S | O | O | O | S | F | OF |
| 1120a-l | Y*AG | S | S | O | O | O | S | OEt | OH |
| 1121a-l | Y*AG | O | O | S | O | S | O | OH | OH |
| 1122a-l | Y*AG | O | O | S | O | S | O | OMe | OH |
| 1123a-l | Y*AG | O | O | S | O | S | O | H | OH |
| 1124a-l | Y*AG | O | O | S | O | S | O | F | OH |
| 1125a-l | Y*AG | O | O | S | O | S | O | OEt | OH |
| 1126a-l | Y*AG | S | O | S | O | S | O | OH | OH |
| 1127a-l | Y*AG | S | O | S | O | S | O | OMe | OH |
| 1128a-l | Y*AG | S | O | S | O | S | O | H | OH |
| 1129a-l | Y*AG | S | O | S | O | S | O | F | OH |
| 1130a-l | Y*AG | S | O | S | O | S | O | OEt | OH |
| 1131a-l | Y*AG | S | S | S | O | S | O | OH | OH |
| 1132a-l | Y*AG | S | S | S | O | S | O | OMe | OH |
| 1133a-l | Y*AG | S | S | S | O | S | O | H | OH |
| 1134a-l | Y*AG | S | S | S | O | S | O | F | OH |
| 1135a-l | Y*AG | S | S | S | O | S | O | OEt | OH |
| 1136a-l | Y*AG | O | O | O | S | O | S | OH | OH |
| 1137a-l | Y*AG | O | O | O | S | O | S | OMe | OH |
| 1138a-l | Y*AG | O | O | O | S | O | S | H | OH |
| 1139a-l | Y*AG | O | O | O | S | O | S | F | OH |
| 1140a-l | Y*AG | O | O | O | S | O | S | OEt | OH |
| 1141a-l | Y*AG | S | O | O | S | O | S | OH | OH |
| 1142a-l | Y*AG | S | O | O | S | O | S | OMe | OH |
| 1143a-l | Y*AG | S | O | O | S | O | S | H | OH |
| 1144a-l | Y*AG | S | O | O | S | O | S | F | OH |
| 1145a-l | Y*AG | S | O | O | S | O | S | OEt | OH |
| 1146a-l | Y*AG | S | S | O | S | O | S | OH | OH |
| 1147a-l | Y*AG | S | S | O | S | O | S | OMe | OH |
| 1148a-l | Y*AG | S | S | O | S | O | S | H | OH |
| 1149a-l | Y*AG | S | S | O | S | O | S | F | OH |
| 1150a-l | Y*AG | S | S | O | S | O | S | OEt | OH |
| 1151a-l | Y*AG | O | O | O | O | O | O | OH | OMe |
| 1152a-l | Y*AG | O | O | O | O | O | O | OMe | OMe |
| 1153a-l | Y*AG | O | O | O | O | O | O | H | OMe |
| 1154a-l | Y*AG | O | O | O | O | O | O | F | OMe |
| 1155a-l | Y*AG | O | O | O | O | O | O | OEt | OMe |
| 1156a-l | Y*AG | S | O | O | O | O | O | OH | OMe |
| 1157a-l | Y*AG | S | O | O | O | O | O | OMe | OMe |
| 1158a-l | Y*AG | S | O | O | O | O | O | H | OMe |
| 1159a-l | Y*AG | S | O | O | O | O | O | F | OMe |
| 1160a-l | Y*AG | S | O | O | O | O | O | OEt | OMe |
| 1161a-l | Y*AG | S | S | O | O | O | O | OH | OMe |
| 1162a-l | Y*AG | S | S | O | O | O | O | OMe | OMe |
| 1163a-l | Y*AG | S | S | O | O | O | O | H | OMe |
| 1164a-l | Y*AG | S | S | O | O | O | O | F | OMe |
| 1165a-l | Y*AG | S | S | O | O | O | O | OEt | OMe |
| 1166a-l | Y*AG | O | O | S | O | O | O | OH | OMe |
| 1167a-l | Y*AG | O | O | S | O | O | O | OMe | OMe |
| 1168a-l | Y*AG | O | O | S | O | O | O | H | OMe |
| 1169a-l | Y*AG | O | O | S | O | O | O | F | OMe |
| 1170a-l | Y*AG | O | O | S | O | O | O | OEt | OMe |
| 1171a-l | Y*AG | S | O | S | O | O | O | OH | OMe |
| 1172a-l | Y*AG | S | O | S | O | O | O | OMe | OMe |
| 1173a-l | Y*AG | S | O | S | O | O | O | H | OMe |
| 1174a-l | Y*AG | S | O | S | O | O | O | F | OMe |
| 1175a-l | Y*AG | S | O | S | O | O | O | OEt | OMe |
| 1176a-l | Y*AG | S | S | S | O | O | O | OH | OMe |
| 1177a-l | Y*AG | S | S | S | O | O | O | OMe | OMe |
| 1178a-l | Y*AG | S | S | S | O | O | O | H | OMe |
| 1179a-l | Y*AG | S | S | S | O | O | O | F | OMe |
| 1180a-l | Y*AG | S | S | S | O | O | O | OEt | OMe |
| 1181a-l | Y*AG | O | O | S | S | O | O | OH | OMe |
| 1182a-l | Y*AG | O | O | S | S | O | O | OMe | OMe |
| 1183a-l | Y*AG | O | O | S | S | O | O | H | OMe |
| 1184a-l | Y*AG | O | O | S | S | O | O | F | OMe |
| 1185a-l | Y*AG | O | O | S | S | O | O | OEt | OMe |
| 1186a-l | Y*AG | S | O | S | S | O | O | OH | OMe |
| 1187a-l | Y*AG | S | O | S | S | O | O | OMe | OMe |
| 1188a-l | Y*AG | S | O | S | S | O | O | H | OMe |
| 1189a-l | Y*AG | S | O | S | S | O | O | F | OMe |
| 1190a-l | Y*AG | S | O | S | S | O | O | OEt | OMe |
| 1191a-l | Y*AG | S | S | S | S | O | O | OH | OMe |
| 1192a-l | Y*AG | S | S | S | S | O | O | OMe | OMe |
| 1193a-l | Y*AG | S | S | S | S | O | O | H | OMe |
| 1194a-l | Y*AG | S | S | S | S | O | O | F | OMe |
| 1195a-l | Y*AG | S | S | S | S | O | O | OEt | OMe |
| 1196a-l | Y*AG | O | O | S | S | S | O | OH | OMe |
| 1197a-l | Y*AG | O | O | S | S | S | O | OMe | OMe |
| 1198a-l | Y*AG | O | O | S | S | S | O | H | OMe |
| 1199a-l | Y*AG | O | O | S | S | S | O | F | OMe |
| 1200a-l | Y*AG | O | O | S | S | S | O | OEt | OMe |
| 1201a-l | Y*AG | S | O | S | S | S | O | OH | OMe |
| 1202a-l | Y*AG | S | O | S | S | S | O | OMe | OMe |
| 1203a-l | Y*AG | S | O | S | S | S | O | H | OMe |
| 1204a-l | Y*AG | S | O | S | S | S | O | F | OMe |
| 1205a-l | Y*AG | S | O | S | S | S | O | OEt | OMe |
| 1206a-l | Y*AG | S | S | S | S | S | O | OH | OMe |
| 1207a-l | Y*AG | S | S | S | S | S | O | OMe | OMe |
| 1208a-l | Y*AG | S | S | S | S | S | O | H | OMe |
| 1209a-l | Y*AG | S | S | S | S | S | O | F | OMe |
| 1210a-l | Y*AG | S | S | S | S | S | O | OEt | OMe |
| 1211a-l | Y*AG | O | O | S | S | S | S | OH | OMe |
| 1212a-l | Y*AG | O | O | S | S | S | S | OMe | OMe |
| 1213a-l | Y*AG | O | O | S | S | S | S | H | OMe |
| 1214a-l | Y*AG | O | O | S | S | S | S | F | OMe |
| 1215a-l | Y*AG | O | O | S | S | S | S | OEt | OMe |
| 1216a-l | Y*AG | S | O | S | S | S | S | OH | OMe |
| 1217a-l | Y*AG | S | O | S | S | S | S | OMe | OMe |
| 1218a-l | Y*AG | S | O | S | S | S | S | H | OMe |
| 1219a-l | Y*AG | S | O | S | S | S | S | F | OMe |
| 1220a-l | Y*AG | S | O | S | S | S | S | OEt | OMe |
| 1221a-l | Y*AG | S | S | S | S | S | S | OH | OMe |
| 1222a-l | Y*AG | S | S | S | S | S | S | OMe | OMe |
| 1223a-l | Y*AG | S | S | S | S | S | S | H | OMe |
| 1224a-l | Y*AG | S | S | S | S | S | S | F | OMe |
| 1225a-l | Y*AG | S | S | S | S | S | S | OEt | OMe |
| 1226a-l | Y*AG | O | O | O | S | S | S | OH | OMe |
| 1227a-l | Y*AG | O | O | O | S | S | S | OMe | OMe |
| 1228a-l | Y*AG | O | O | O | S | S | S | H | OMe |
| 1229a-l | Y*AG | O | O | O | S | S | S | F | OMe |
| 1230a-l | Y*AG | O | O | O | S | S | S | OEt | OMe |
| 1231a-l | Y*AG | S | O | O | S | S | S | OH | OMe |
| 1232a-l | Y*AG | S | O | O | S | S | S | OMe | OMe |
| 1233a-l | Y*AG | S | O | O | S | S | S | H | OMe |
| 1234a-l | Y*AG | S | O | O | S | S | S | F | OMe |
| 1235a-l | Y*AG | S | O | O | S | S | S | OEt | OMe |
| 1236a-l | Y*AG | S | S | O | S | S | S | OH | OMe |
| 1237a-l | Y*AG | S | S | O | S | S | S | OMe | OMe |
| 1238a-l | Y*AG | S | S | O | S | S | S | H | OMe |
| 1239a-l | Y*AG | S | S | O | S | S | S | F | OMe |
| 1240a-l | Y*AG | S | S | O | S | S | S | OEt | OMe |
| 1241a-l | Y*AG | O | O | O | O | S | S | OH | OMe |
| 1242a-l | Y*AG | O | O | O | O | S | S | OMe | OMe |
| 1243a-l | Y*AG | O | O | O | O | S | S | H | OMe |
| 1244a-l | Y*AG | O | O | O | O | S | S | F | OMe |
| 1245a-l | Y*AG | O | O | O | O | S | S | OEt | OMe |
| 1246a-l | Y*AG | S | O | O | O | S | S | OH | OMe |
| 1247a-l | Y*AG | S | O | O | O | S | S | OMe | OMe |
| 1248a-l | Y*AG | S | O | O | O | S | S | H | OMe |
| 1249a-l | Y*AG | S | O | O | O | S | S | F | OMe |
| 1250a-l | Y*AG | S | O | O | O | S | S | OEt | OMe |
| 1251a-l | Y*AG | S | S | O | O | S | S | OH | OMe |
| 1252a-l | Y*AG | S | S | O | O | S | S | OMe | OMe |
| 1253a-l | Y*AG | S | S | O | O | S | S | H | OMe |
| 1254a-l | Y*AG | S | S | O | O | S | S | F | OMe |
| 1255a-l | Y*AG | S | S | O | O | S | S | OEt | OMe |
| 1256a-l | Y*AG | O | O | O | O | O | S | OH | OMe |
| 1257a-l | Y*AG | O | O | O | O | O | S | OMe | OMe |
| 1258a-l | Y*AG | O | O | O | O | O | S | H | OMe |
| 1259a-l | Y*AG | O | O | O | O | O | S | F | OMe |
| 1260a-l | Y*AG | O | O | O | O | O | S | OEt | OMe |
| 1261a-l | Y*AG | S | O | O | O | O | S | OH | OMe |
| 1262a-l | Y*AG | S | O | O | O | O | S | OMe | OMe |
| 1263a-l | Y*AG | S | O | O | O | O | S | H | OMe |
| 1264a-l | Y*AG | S | O | O | O | O | S | F | OMe |
| 1265a-l | Y*AG | S | O | O | O | O | S | OEt | OMe |
| 1266a-l | Y*AG | S | S | O | O | O | S | OH | OMe |
| 1267a-l | Y*AG | S | S | O | O | O | S | OMe | OMe |
| 1268a-l | Y*AG | S | S | O | O | O | S | H | OMe |
| 1269a-l | Y*AG | S | S | O | O | O | S | F | OMe |
| 1270a-l | Y*AG | S | S | O | O | O | S | OEt | OMe |
| 1271a-l | Y*AG | O | O | S | O | S | O | OH | OMe |
| 1272a-l | Y*AG | O | O | S | O | S | O | OMe | OMe |
| 1273a-l | Y*AG | O | O | S | O | S | O | H | OMe |
| 1274a-l | Y*AG | O | O | S | O | S | O | F | OMe |
| 1275a-l | Y*AG | O | O | S | O | S | O | OEt | OMe |
| 1276a-l | Y*AG | S | O | S | O | S | O | OH | OMe |
| 1277a-l | Y*AG | S | O | S | O | S | O | OMe | OMe |
| 1278a-l | Y*AG | S | O | S | O | S | O | H | OMe |
| 1279a-l | Y*AG | S | O | S | O | S | O | F | OMe |
| 1280a-l | Y*AG | S | O | S | O | S | O | OEt | OMe |
| 1281a-l | Y*AG | S | S | S | O | S | O | OH | OMe |
| 1282a-l | Y*AG | S | S | S | O | S | O | OMe | OMe |
| 1283a-l | Y*AG | S | S | S | O | S | O | H | OMe |
| 1284a-l | Y*AG | S | S | S | O | S | O | F | OMe |
| 1285a-l | Y*AG | S | S | S | O | S | O | OEt | OMe |
| 1286a-l | Y*AG | O | O | O | S | O | S | OH | OMe |
| 1287a-l | Y*AG | O | O | O | S | O | S | OMe | OMe |
| 1288a-l | Y*AG | O | O | O | S | O | S | H | OMe |
| 1289a-l | Y*AG | O | O | O | S | O | S | F | OMe |
| 1290a-l | Y*AG | O | O | O | S | O | S | OEt | OMe |
| 1291a-l | Y*AG | S | O | O | S | O | S | OH | OMe |
| 1292a-l | Y*AG | S | O | O | S | O | S | OMe | OMe |
| 1293a-l | Y*AG | S | O | O | S | O | S | H | OMe |
| 1294a-l | Y*AG | S | O | O | S | O | S | F | OMe |
| 1295a-l | Y*AG | S | O | O | S | O | S | OEt | OMe |
| 1296a-l | Y*AG | S | S | O | S | O | S | OH | OMe |
| 1297a-l | Y*AG | S | S | O | S | O | S | OMe | OMe |
| 1298a-l | Y*AG | S | S | O | S | O | S | H | OMe |
| 1299a-l | Y*AG | S | S | O | S | O | S | F | OMe |
| 1300a-l | Y*AG | S | S | O | S | O | S | OEt | OMe |
| 1301a-l | Y*AA | O | O | O | O | O | O | OH | OH |
| 1302a-l | Y*AA | O | O | O | O | O | O | OMe | OH |
| 1303a-l | Y*AA | O | O | O | O | O | O | H | OH |
| 1304a-l | Y*AA | O | O | O | O | O | O | F | OH |
| 1305a-l | Y*AA | O | O | O | O | O | O | OEt | OH |
| 1306a-l | Y*AA | S | O | O | O | O | O | OH | OH |
| 1307a-l | Y*AA | S | O | O | O | O | O | OMe | OH |
| 1308a-l | Y*AA | S | O | O | O | O | O | H | OH |
| 1309a-l | Y*AA | S | O | O | O | O | O | F | OH |
| 1310a-l | Y*AA | S | O | O | O | O | O | OEt | OH |
| 1311a-l | Y*AA | S | S | O | O | O | O | OH | OH |
| 1312a-l | Y*AA | S | S | O | O | O | O | OMe | OH |
| 1313a-l | Y*AA | S | S | O | O | O | O | H | OH |
| 1314a-l | Y*AA | S | S | O | O | O | O | F | OH |
| 1315a-l | Y*AA | S | S | O | O | O | O | OEt | OH |
| 1316a-l | Y*AA | O | O | S | O | O | O | OH | OH |
| 1317a-l | Y*AA | O | O | S | O | O | O | OMe | OH |
| 1318a-l | Y*AA | O | O | S | O | O | O | H | OH |
| 1319a-l | Y*AA | O | O | S | O | O | O | F | OH |
| 1320a-l | Y*AA | O | O | S | O | O | O | OEt | OH |
| 1321a-l | Y*AA | S | O | S | O | O | O | OH | OH |
| 1322a-l | Y*AA | S | O | S | O | O | O | OMe | OH |
| 1323a-l | Y*AA | S | O | S | O | O | O | H | OH |
| 1324a-l | Y*AA | S | O | S | O | O | O | F | OH |
| 1325a-l | Y*AA | S | O | S | O | O | O | OEt | OH |
| 1326a-l | Y*AA | S | S | S | O | O | O | OH | OH |
| 1327a-l | Y*AA | S | S | S | O | O | O | OMe | OH |
| 1328a-l | Y*AA | S | S | S | O | O | O | H | OH |
| 1329a-l | Y*AA | S | S | S | O | O | O | F | OH |
| 1330a-l | Y*AA | S | S | S | O | O | O | OEt | OH |
| 1331a-l | Y*AA | O | O | S | S | O | O | OH | OH |
| 1332a-l | Y*AA | O | O | S | S | O | O | OMe | OH |
| 1333a-l | Y*AA | O | O | S | S | O | O | H | OH |
| 1334a-l | Y*AA | O | O | S | S | O | O | F | OH |
| 1335a-l | Y*AA | O | O | S | S | O | O | OEt | OH |
| 1336a-l | Y*AA | S | O | S | S | O | O | OH | OH |
| 1337a-l | Y*AA | S | O | S | S | O | O | OMe | OH |
| 1338a-l | Y*AA | S | O | S | S | O | O | H | OH |
| 1339a-l | Y*AA | S | O | S | S | O | O | F | OH |
| 1340a-l | Y*AA | S | O | S | S | O | O | OEt | OH |
| 1341a-l | Y*AA | S | S | S | S | O | O | OH | OH |
| 1342a-l | Y*AA | S | S | S | S | O | O | OMe | OH |
| 1343a-l | Y*AA | S | S | S | S | O | O | H | OH |
| 1344a-l | Y*AA | S | S | S | S | O | O | F | OH |
| 1345a-l | Y*AA | S | S | S | S | O | O | OEt | OH |
| 1346a-l | Y*AA | O | O | S | S | S | O | OH | OH |
| 1347a-l | Y*AA | O | O | S | S | S | O | OMe | OH |
| 1348a-l | Y*AA | O | O | S | S | S | O | H | OH |
| 1349a-l | Y*AA | O | O | S | S | S | O | F | OH |
| 1350a-l | Y*AA | O | O | S | S | S | O | OEt | OH |
| 1351a-l | Y*AA | S | O | S | S | S | O | OH | OH |
| 1352a-l | Y*AA | S | O | S | S | S | O | OMe | OH |
| 1353a-l | Y*AA | S | O | S | S | S | O | H | OH |
| 1354a-l | Y*AA | S | O | S | S | S | O | F | OH |
| 1355a-l | Y*AA | S | O | S | S | S | O | OEt | OH |
| 1356a-l | Y*AA | S | S | S | S | S | O | OH | OH |
| 1357a-l | Y*AA | S | S | S | S | S | O | OMe | OH |
| 1358a-l | Y*AA | S | S | S | S | S | O | H | OH |
| 1359a-l | Y*AA | S | S | S | S | S | O | F | OH |
| 1360a-l | Y*AA | S | S | S | S | S | O | OEt | OH |
| 1361a-l | Y*AA | O | O | S | S | S | S | OH | OH |
| 1362a-l | Y*AA | O | O | S | S | S | S | OMe | OH |
| 1363a-l | Y*AA | O | O | S | S | S | S | H | OH |
| 1364a-l | Y*AA | O | O | S | S | S | S | F | OH |
| 1365a-l | Y*AA | O | O | S | S | S | S | OEt | OH |
| 1366a-l | Y*AA | S | O | S | S | S | S | OH | OH |
| 1367a-l | Y*AA | S | O | S | S | S | S | OMe | OH |
| 1368a-l | Y*AA | S | O | S | S | S | S | H | OH |
| 1369a-l | Y*AA | S | O | S | S | S | S | F | OH |
| 1370a-l | Y*AA | S | O | S | S | S | S | OEt | OH |
| 1371a-l | Y*AA | S | S | S | S | S | S | OH | OH |
| 1372a-l | Y*AA | S | S | S | S | S | S | OMe | OH |
| 1373a-l | Y*AA | S | S | S | S | S | S | H | OH |
| 1374a-l | Y*AA | S | S | S | S | S | S | F | OH |
| 1375a-l | Y*AA | S | S | S | S | S | S | OEt | OH |
| 1376a-l | Y*AA | O | O | O | S | S | S | OH | OH |
| 1377a-l | Y*AA | O | O | O | S | S | S | OMe | OH |
| 1378a-l | Y*AA | O | O | O | S | S | S | H | OH |
| 1379a-l | Y*AA | O | O | O | S | S | S | F | OH |
| 1380a-l | Y*AA | O | O | O | S | S | S | OEt | OH |
| 1381a-l | Y*AA | S | O | O | S | S | S | OH | OH |
| 1382a-l | Y*AA | S | O | O | S | S | S | OMe | OH |
| 1383a-l | Y*AA | S | O | O | S | S | S | H | OH |
| 1384a-l | Y*AA | S | O | O | S | S | S | F | OH |
| 1385a-l | Y*AA | S | O | O | S | S | S | OEt | OH |
| 1386a-l | Y*AA | S | S | O | S | S | S | OH | OH |
| 1387a-l | Y*AA | S | S | O | S | S | S | OMe | OH |
| 1388a-l | Y*AA | S | S | O | S | S | S | H | OH |
| 1389a-l | Y*AA | S | S | O | S | S | S | F | OH |
| 1390a-l | Y*AA | S | S | O | S | S | S | OEt | OH |
| 1391a-l | Y*AA | O | O | O | O | S | S | OH | OH |
| 1392a-l | Y*AA | O | O | O | O | S | S | OMe | OH |
| 1393a-l | Y*AA | O | O | O | O | S | S | H | OH |
| 1394a-l | Y*AA | O | O | O | O | S | S | F | OH |
| 1395a-l | Y*AA | O | O | O | O | S | S | OEt | OH |
| 1396a-l | Y*AA | S | O | O | O | S | S | OH | OH |
| 1397a-l | Y*AA | S | O | O | O | S | S | OMe | OH |
| 1398a-l | Y*AA | S | O | O | O | S | S | H | OH |
| 1399a-l | Y*AA | S | O | O | O | S | S | F | OH |
| 1400a-l | Y*AA | S | O | O | O | S | S | OEt | OH |
| 1401a-l | Y*AA | S | S | O | O | S | S | OH | OH |
| 1402a-l | Y*AA | S | S | O | O | S | S | OMe | OH |
| 1403a-l | Y*AA | S | S | O | O | S | S | H | OH |
| 1404a-l | Y*AA | S | S | O | O | S | S | F | OH |
| 1405a-l | Y*AA | S | S | O | O | S | S | OEt | OH |
| 1406a-l | Y*AA | O | O | O | O | O | S | OH | OH |
| 1407a-l | Y*AA | O | O | O | O | O | S | OMe | OH |
| 1408a-l | Y*AA | O | O | O | O | O | S | H | OH |
| 1409a-l | Y*AA | O | O | O | O | O | S | F | OH |
| 1410a-l | Y*AA | O | O | O | O | O | S | OEt | OH |
| 1411a-l | Y*AA | S | O | O | O | O | S | OH | OH |
| 1412a-l | Y*AA | S | O | O | O | O | S | OMe | OH |
| 1413a-l | Y*AA | S | O | O | O | O | S | H | OH |
| 1414a-l | Y*AA | S | O | O | O | O | S | F | OH |
| 1415a-l | Y*AA | S | O | O | O | O | S | OEt | OH |
| 1416a-l | Y*AA | S | S | O | O | O | S | OH | OH |
| 1417a-l | Y*AA | S | S | O | O | O | S | OMe | OH |
| 1418a-l | Y*AA | S | S | O | O | O | S | H | OH |
| 1419a-l | Y*AA | S | S | O | O | O | S | F | OH |
| 1420a-l | Y*AA | S | S | O | O | O | S | OEt | OH |
| 1421a-l | Y*AA | O | O | S | O | S | O | OH | OH |
| 1422a-l | Y*AA | O | O | S | O | S | O | OMe | OH |
| 1423a-l | Y*AA | O | O | S | O | S | O | H | OH |
| 1424a-l | Y*AA | O | O | S | O | S | O | F | OH |
| 1425a-l | Y*AA | O | O | S | O | S | O | OEt | OH |
| 1426a-l | Y*AA | S | O | S | O | S | O | OH | OH |
| 1427a-l | Y*AA | S | O | S | O | S | O | OMe | OH |
| 1428a-l | Y*AA | S | C | S | O | S | O | H | OH |
| 1429a-l | Y*AA | S | O | S | O | S | O | F | OH |
| 1430a-l | Y*AA | S | O | S | O | S | O | OEt | OH |
| 1431a-l | Y*AA | S | S | S | O | S | O | OH | OH |
| 1432a-l | Y*AA | S | S | S | O | S | O | OMe | OH |
| 1433a-l | Y*AA | S | S | S | O | S | O | H | OH |
| 1434a-l | Y*AA | S | S | S | O | S | O | F | OH |
| 1435a-l | Y*AA | S | S | S | O | S | O | OEt | OH |
| 1436a-l | Y*AA | O | O | O | S | O | S | OH | OH |
| 1437a-l | Y*AA | O | O | O | S | O | S | OMe | OH |
| 1438a-l | Y*AA | O | O | O | S | O | S | H | OH |
| 1439a-l | Y*AA | O | O | O | S | O | S | F | OH |
| 1440a-l | Y*AA | O | O | O | S | O | S | OEt | OH |
| 1441a-l | Y*AA | S | O | O | S | O | S | OH | OH |
| 1442a-l | Y*AA | S | O | O | S | O | S | OMe | OH |
| 1443a-l | Y*AA | S | O | O | S | O | S | H | OH |
| 1444a-l | Y*AA | S | O | O | S | O | S | F | OH |
| 1445a-l | Y*AA | S | O | O | S | O | S | OEt | OH |
| 1446a-l | Y*AA | S | S | O | S | O | S | OH | OH |
| 1447a-l | Y*AA | S | S | O | S | O | S | OMe | OH |
| 1448a-l | Y*AA | S | S | O | S | O | S | H | OH |
| 1449a-l | Y*AA | S | S | O | S | O | S | F | OH |
| 1450a-l | Y*AA | S | S | O | S | O | S | OEt | OH |
| 1451a-l | Y*AA | O | O | O | O | O | O | OH | OMe |
| 1452a-l | Y*AA | O | O | O | O | O | O | OMe | OMe |
| 1453a-l | Y*AA | O | O | O | O | O | O | H | OMe |
| 1454a-l | Y*AA | O | O | O | O | O | O | F | OMe |
| 1455a-l | Y*AA | O | O | O | O | O | O | OEt | OMe |
| 1456a-l | Y*AA | S | O | O | O | O | O | OH | OMe |
| 1457a-l | Y*AA | S | O | O | O | O | O | OMe | OMe |
| 1458a-l | Y*AA | S | O | O | O | O | O | H | OMe |
| 1459a-l | Y*AA | S | O | O | O | O | O | F | OMe |
| 1460a-l | Y*AA | S | O | O | O | O | O | OEt | OMe |
| 1461a-l | Y*AA | S | S | O | O | O | O | OH | OMe |
| 1462a-l | Y*AA | S | S | O | O | O | O | OMe | OMe |
| 1463a-l | Y*AA | S | S | O | O | O | O | H | OMe |
| 1464a-l | Y*AA | S | S | O | O | O | O | F | OMe |
| 1465a-l | Y*AA | S | S | O | O | O | O | OEt | OMe |
| 1466a-l | Y*AA | O | O | S | O | O | O | OH | OMe |
| 1467a-l | Y*AA | O | O | S | O | O | O | OMe | OMe |
| 1468a-l | Y*AA | O | O | S | O | O | O | H | OMe |
| 1469a-l | Y*AA | O | O | S | O | O | O | F | OMe |
| 1470a-l | Y*AA | O | O | S | O | O | O | OEt | OMe |
| 1471a-l | Y*AA | S | O | S | O | O | O | OH | OMe |
| 1472a-l | Y*AA | S | O | S | O | O | O | OMe | OMe |
| 1473a-l | Y*AA | S | O | S | O | O | O | H | OMe |
| 1474a-l | Y*AA | S | O | S | O | O | O | F | OMe |
| 1475a-l | Y*AA | S | O | S | O | O | O | OEt | OMe |
| 1476a-l | Y*AA | S | S | S | O | O | O | OH | OMe |
| 1477a-l | Y*AA | S | S | S | O | O | O | OMe | OMe |
| 1478a-l | Y*AA | S | S | S | O | O | O | H | OMe |
| 1479a-l | Y*AA | S | S | S | O | O | O | F | OMe |
| 1480a-l | Y*AA | S | S | S | O | O | O | OEt | OMe |
| 1481a-l | Y*AA | O | O | S | S | O | O | OH | OMe |
| 1482a-l | Y*AA | O | O | S | S | O | O | OMe | OMe |
| 1483a-l | Y*AA | O | O | S | S | O | O | H | OMe |
| 1484a-l | Y*AA | O | O | S | S | O | O | F | OMe |
| 1485a-l | Y*AA | O | O | S | S | O | O | OEt | OMe |
| 1486a-l | Y*AA | S | O | S | S | O | O | OH | OMe |
| 1487a-l | Y*AA | S | O | S | S | O | O | OMe | OMe |
| 1488a-l | Y*AA | S | O | S | S | O | O | H | OMe |
| 1489a-l | Y*AA | S | O | S | S | O | O | F | OMe |
| 1490a-l | Y*AA | S | O | S | S | O | O | OEt | OMe |
| 1491a-l | Y*AA | S | S | S | S | O | O | OH | OMe |
| 1492a-l | Y*AA | S | S | S | S | O | O | OMe | OMe |
| 1493a-l | Y*AA | S | S | S | S | O | O | H | OMe |
| 1494a-l | Y*AA | S | S | S | S | O | O | F | OMe |
| 1495a-l | Y*AA | S | S | S | S | O | O | OEt | OMe |
| 1496a-l | Y*AA | O | O | S | S | S | O | OH | OMe |
| 1497a-l | Y*AA | O | O | S | S | S | O | OMe | OMe |
| 1498a-l | Y*AA | O | O | S | S | S | O | H | OMe |
| 1499a-l | Y*AA | O | O | S | S | S | O | F | OMe |
| 1500a-l | Y*AA | O | O | S | S | S | O | OEt | OMe |
| 1501a-l | Y*AA | S | O | S | S | S | O | OH | OMe |
| 1502a-l | Y*AA | S | O | S | S | S | O | OMe | OMe |
| 1503a-l | Y*AA | S | O | S | S | S | O | H | OMe |
| 1504a-l | Y*AA | S | O | S | S | S | O | F | OMe |
| 1505a-l | Y*AA | S | O | S | S | S | O | OEt | OMe |
| 1506a-l | Y*AA | S | S | S | S | S | O | OH | OMe |
| 1507a-l | Y*AA | S | S | S | S | S | O | OMe | OMe |
| 1508a-l | Y*AA | S | S | S | S | S | O | H | OMe |
| 1509a-l | Y*AA | S | S | S | S | S | O | F | OMe |
| 1510a-l | Y*AA | S | S | S | S | S | O | OEt | OMe |
| 1511a-l | Y*AA | O | O | S | S | S | S | OH | OMe |
| 1512a-l | Y*AA | O | O | S | S | S | S | OMe | OMe |
| 1513a-l | Y*AA | O | O | S | S | S | S | H | OMe |
| 1514a-l | Y*AA | O | O | S | S | S | S | F | OMe |
| 1515a-l | Y*AA | O | O | S | S | S | S | OEt | OMe |
| 1516a-l | Y*AA | S | O | S | S | S | S | OH | OMe |
| 1517a-l | Y*AA | S | O | S | S | S | S | OMe | OMe |
| 1518a-l | Y*AA | S | O | S | S | S | S | H | OMe |
| 1519a-l | Y*AA | S | O | S | S | S | S | F | OMe |
| 1520a-l | Y*AA | S | O | S | S | S | S | OEt | OMe |
| 1521a-l | Y*AA | S | S | S | S | S | S | OH | OMe |
| 1522a-l | Y*AA | S | S | S | S | S | S | OMe | OMe |
| 1523a-l | Y*AA | S | S | S | S | S | S | H | OMe |
| 1524a-l | Y*AA | S | S | S | S | S | S | F | OMe |
| 1525a-l | Y*AA | S | S | S | S | S | S | OEt | OMe |
| 1526a-l | Y*AA | O | O | O | S | S | S | OH | OMe |
| 1527a-l | Y*AA | O | O | O | S | S | S | OMe | OMe |
| 1528a-l | Y*AA | O | O | O | S | S | S | H | OMe |
| 1529a-l | Y*AA | O | O | O | S | S | S | F | OMe |
| 1530a-l | Y*AA | O | O | O | S | S | S | OEt | OMe |
| 1531a-l | Y*AA | S | O | O | S | S | S | OH | OMe |
| 1532a-l | Y*AA | S | O | O | S | S | S | OMe | OMe |
| 1533a-l | Y*AA | S | O | O | S | S | S | H | OMe |
| 1534a-l | Y*AA | S | O | O | S | S | S | F | OMe |
| 1535a-l | Y*AA | S | O | O | S | S | S | OEt | OMe |
| 1536a-l | Y*AA | S | S | O | S | S | S | OH | OMe |
| 1537a-l | Y*AA | S | S | O | S | S | S | OMe | OMe |
| 1538a-l | Y*AA | S | S | O | S | S | S | H | OMe |
| 1539a-l | Y*AA | S | S | O | S | S | S | F | OMe |
| 1540a-l | Y*AA | S | S | O | S | S | S | OEt | OMe |
| 1541a-l | Y*AA | O | O | O | O | S | S | OH | OMe |
| 1542a-l | Y*AA | O | O | O | O | S | S | OMe | OMe |
| 1543a-l | Y*AA | O | O | O | O | S | S | H | OMe |
| 1544a-l | Y*AA | O | O | O | O | S | S | F | OMe |
| 1545a-l | Y*AA | C | O | O | O | S | S | OEt | OMe |
| 1546a-l | Y*AA | S | O | O | O | S | S | OH | OMe |
| 1547a-l | Y*AA | S | O | O | O | S | S | OMe | OMe |
| 1548a-l | Y*AA | S | O | O | O | S | S | H | OMe |
| 1549a-l | Y*AA | S | O | O | O | S | S | F | OMe |
| 1550a-l | Y*AA | S | O | O | O | S | S | OEt | OMe |
| 1551a-l | Y*AA | S | S | O | O | S | S | OH | OMe |
| 1552a-l | Y*AA | S | S | O | O | S | S | OMe | OMe |
| 1553a-l | Y*AA | S | S | O | O | S | S | H | OMe |
| 1554a-l | Y*AA | S | S | O | O | S | S | F | OMe |
| 1555a-l | Y*AA | S | S | O | O | S | S | OEt | OMe |
| 1556a-l | Y*AA | O | O | O | O | O | S | OH | OMe |
| 1557a-l | Y*AA | O | O | O | O | O | S | OMe | OMe |
| 1558a-l | Y*AA | O | O | O | O | O | S | H | OMe |
| 1559a-l | Y*AA | O | O | O | O | O | S | F | OMe |
| 1560a-l | Y*AA | O | O | O | O | O | S | OEt | OMe |
| 1561a-l | Y*AA | S | O | O | O | O | S | OH | OMe |
| 1562a-l | Y*AA | S | O | O | O | O | S | OMe | OMe |
| 1563a-l | Y*AA | S | O | O | O | O | S | H | OMe |
| 1564a-l | Y*AA | S | O | O | O | O | S | F | OMe |
| 1565a-l | Y*AA | S | O | O | O | O | S | OEt | OMe |
| 1566a-l | Y*AA | S | S | O | O | O | S | OH | OMe |
| 1567a-l | Y*AA | S | S | O | O | O | S | OMe | OMe |
| 1568a-l | Y*AA | S | S | O | O | O | S | H | OMe |
| 1569a-l | Y*AA | S | S | O | O | O | S | F | OMe |
| 1570a-l | Y*AA | S | S | O | O | O | S | OEt | OMe |
| 1571a-l | Y*AA | O | O | S | O | S | O | OH | OMe |
| 1572a-l | Y*AA | O | O | S | O | S | O | OMe | OMe |
| 1573a-l | Y*AA | O | O | S | O | S | O | H | OMe |
| 1574a-l | Y*AA | O | O | S | O | S | O | F | OMe |
| 1575a-l | Y*AA | O | O | S | O | S | O | OEt | OMe |
| 1576a-l | Y*AA | S | O | S | O | S | O | OH | OMe |
| 1577a-l | Y*AA | S | O | S | O | S | O | OMe | OMe |
| 1578a-l | Y*AA | S | O | S | O | S | O | H | OMe |
| 1579a-l | Y*AA | S | O | S | O | S | O | F | OMe |
| 1580a-l | Y*AA | S | O | S | O | S | O | OEt | OMe |
| 1581a-l | Y*AA | S | S | S | O | S | O | OH | OMe |
| 1582a-l | Y*AA | S | S | S | O | S | O | OMe | OMe |
| 1583a-l | Y*AA | S | S | S | O | S | O | H | OMe |
| 1584a-l | Y*AA | S | S | S | O | S | O | F | OMe |
| 1585a-l | Y*AA | S | S | S | O | S | O | OEt | OMe |
| 1586a-l | Y*AA | O | O | O | S | O | S | OH | OMe |
| 1587a-l | Y*AA | O | O | O | S | O | S | OMe | OMe |
| 1588a-l | Y*AA | O | O | O | S | O | S | H | OMe |
| 1589a-l | Y*AA | O | O | O | S | O | S | F | OMe |
| 1590a-l | Y*AA | O | O | O | S | O | S | OEt | OMe |
| 1591a-l | Y*AA | S | O | O | S | O | S | OH | OMe |
| 1592a-l | Y*AA | S | O | O | S | O | S | OMe | OMe |
| 1593a-l | Y*AA | S | O | O | S | O | S | H | OMe |
| 1594a-l | Y*AA | S | O | O | S | O | S | F | OMe |
| 1595a-l | Y*AA | S | O | O | S | O | S | OEt | OMe |
| 1596a-l | Y*AA | S | S | O | S | O | S | OH | OMe |
| 1597a-l | Y*AA | S | S | O | S | O | S | OMe | OMe |
| 1598a-l | Y*AA | S | S | O | S | O | S | H | OMe |
| 1599a-l | Y*AA | S | S | O | S | O | S | F | OMe |
| 1600a-l | Y*AA | S | S | O | S | O | S | OEt | OMe |
| 1601a-l | Y*AC | O | O | O | O | O | O | OH | OH |
| 1602a-l | Y*AC | O | O | O | O | O | O | OMe | OH |
| 1603a-l | Y*AC | O | O | O | O | O | O | H | OH |
| 1604a-l | Y*AC | O | O | O | O | O | O | F | OH |
| 1605a-l | Y*AC | O | O | O | O | O | O | OEt | OH |
| 1606a-l | Y*AC | S | O | O | O | O | O | OH | OH |
| 1607a-l | Y*AC | S | O | O | O | O | O | OMe | OH |
| 1608a-l | Y*AC | S | O | O | O | O | O | H | OH |
| 1609a-l | Y*AC | S | O | O | O | O | O | F | OH |
| 1610a-l | Y*AC | S | O | O | O | O | O | OEt | OH |
| 1611a-l | Y*AC | S | S | O | O | O | O | OH | OH |
| 1612a-l | Y*AC | S | S | O | O | O | O | OMe | OH |
| 1613a-l | Y*AC | S | S | O | O | O | O | H | OH |
| 1614a-l | Y*AC | S | S | O | O | O | O | F | OH |
| 1615a-l | Y*AC | S | S | O | O | O | O | OEt | OH |
| 1616a-l | Y*AC | O | O | S | O | O | O | OH | OH |
| 1617a-l | Y*AC | O | O | S | O | O | O | OMe | OH |
| 1618a-l | Y*AC | O | O | S | O | O | O | H | OH |
| 1619a-l | Y*AC | O | O | S | O | O | O | F | OH |
| 1620a-l | Y*AC | O | O | S | O | O | O | OEt | OH |
| 1621a-l | Y*AC | S | O | S | O | O | O | OH | OH |
| 1622a-l | Y*AC | S | O | S | O | O | O | OMe | OH |
| 1623a-l | Y*AC | S | O | S | O | O | O | H | OH |
| 1624a-l | Y*AC | S | O | S | O | O | O | F | OH |
| 1625a-l | Y*AC | S | O | S | O | O | O | OEt | OH |
| 1626a-l | Y*AC | S | S | S | O | O | O | OH | OH |
| 1627a-l | Y*AC | S | S | S | O | O | O | OMe | OH |
| 1628a-l | Y*AC | S | S | S | O | O | O | H | OH |
| 1629a-l | Y*AC | S | S | S | O | O | O | F | OH |
| 1630a-l | Y*AC | S | S | S | O | O | O | OEt | OH |
| 1631a-l | Y*AC | O | O | S | S | O | O | OH | OH |
| 1632a-l | Y*AC | O | O | S | S | O | O | OMe | OH |
| 1633a-l | Y*AC | O | O | S | S | O | O | H | OH |
| 1634a-l | Y*AC | O | O | S | S | O | O | F | OH |
| 1635a-l | Y*AC | O | O | S | S | O | O | OEt | OH |
| 1636a-l | Y*AC | S | O | S | S | O | O | OH | OH |
| 1637a-l | Y*AC | S | O | S | S | O | O | OMe | OH |
| 1638a-l | Y*AC | S | O | S | S | O | O | H | OH |
| 1639a-l | Y*AC | S | O | S | S | O | O | F | OH |
| 1640a-l | Y*AC | S | O | S | S | O | O | OEt | OH |
| 1641a-l | Y*AC | S | S | S | S | O | O | OH | OH |
| 1642a-l | Y*AC | S | S | S | S | O | O | OMe | OH |
| 1643a-l | Y*AC | S | S | S | S | O | O | H | OH |
| 1644a-l | Y*AC | S | S | S | S | O | O | F | OH |
| 1645a-l | Y*AC | S | S | S | S | O | O | OEt | OH |
| 1646a-l | Y*AC | O | O | S | S | S | O | OH | OH |
| 1647a-l | Y*AC | O | O | S | S | S | O | OMe | OH |
| 1648a-l | Y*AC | O | O | S | S | S | O | H | OH |
| 1649a-l | Y*AC | O | O | S | S | S | O | F | OH |
| 1650a-l | Y*AC | O | O | S | S | S | O | OEt | OH |
| 1651a-l | Y*AC | S | O | S | S | S | O | OH | OH |
| 1652a-l | Y*AC | S | O | S | S | S | O | OMe | OH |
| 1653a-l | Y*AC | S | O | S | S | S | O | H | OH |
| 1654a-l | Y*AC | S | O | S | S | S | O | F | OH |
| 1655a-l | Y*AC | S | O | S | S | S | O | OEt | OH |
| 1656a-l | Y*AC | S | S | S | S | S | O | OH | OH |
| 1657a-l | Y*AC | S | S | S | S | S | O | OMe | OH |
| 1658a-l | Y*AC | S | S | S | S | S | O | H | OH |
| 1659a-l | Y*AC | S | S | S | S | S | O | F | OH |
| 1660a-l | Y*AC | S | S | S | S | S | O | OEt | OH |
| 1661a-l | Y*AC | O | O | S | S | S | S | OH | OH |
| 1662a-l | Y*AC | O | O | S | S | S | S | OMe | OH |
| 1663a-l | Y*AC | O | O | S | S | S | S | H | OH |
| 1664a-l | Y*AC | O | O | S | S | S | S | F | OH |
| 1665a-l | Y*AC | O | O | S | S | S | S | OEt | OH |
| 1666a-l | Y*AC | S | O | S | S | S | S | OH | OH |
| 1667a-l | Y*AC | S | O | S | S | S | S | OMe | OH |
| 1668a-l | Y*AC | S | O | S | S | S | S | H | OH |
| 1669a-l | Y*AC | S | O | S | S | S | S | F | OH |
| 1670a-l | Y*AC | S | O | S | S | S | S | OEt | OH |
| 1671a-l | Y*AC | S | S | S | S | S | S | OH | OH |
| 1672a-l | Y*AC | S | S | S | S | S | S | OMe | OH |
| 1673a-l | Y*AC | S | S | S | S | S | S | H | OH |
| 1674a-l | Y*AC | S | S | S | S | S | S | F | OH |
| 1675a-l | Y*AC | S | S | S | S | S | S | OEt | OH |
| 1676a-l | Y*AC | O | O | O | S | S | S | OH | OH |
| 1677a-l | Y*AC | O | O | O | S | S | S | OMe | OH |
| 1678a-l | Y*AC | O | O | O | S | S | S | H | OH |
| 1679a-l | Y*AC | O | O | O | S | S | S | F | OH |
| 1680a-l | Y*AC | O | O | O | S | S | S | OEt | OH |
| 1681a-l | Y*AC | S | O | O | S | S | S | OH | OH |
| 1682a-l | Y*AC | S | O | O | S | S | S | OMe | OH |
| 1683a-l | Y*AC | S | O | O | S | S | S | H | OH |
| 1684a-l | Y*AC | S | O | O | S | S | S | F | OH |
| 1685a-l | Y*AC | S | O | O | S | S | S | OEt | OH |
| 1686a-l | Y*AC | S | S | O | S | S | S | OH | OH |
| 1687a-l | Y*AC | S | S | O | S | S | S | OMe | OH |
| 1688a-l | Y*AC | S | S | O | S | S | S | H | OH |
| 1689a-l | Y*AC | S | S | O | S | S | S | F | OH |
| 1690a-l | Y*AC | S | S | O | S | S | S | OEt | OH |
| 1691a-l | Y*AC | O | O | O | O | S | S | OH | OH |
| 1692a-l | Y*AC | O | O | O | O | S | S | OMe | OH |
| 1693a-l | Y*AC | O | O | O | O | S | S | H | OH |
| 1694a-l | Y*AC | O | O | O | O | S | S | F | OH |
| 1695a-l | Y*AC | O | O | O | O | S | S | OEt | OH |
| 1696a-l | Y*AC | S | O | O | O | S | S | OH | OH |
| 1697a-l | Y*AC | S | O | O | O | S | S | OMe | OH |
| 1698a-l | Y*AC | S | O | O | O | S | S | H | OH |
| 1699a-l | Y*AC | S | O | O | O | S | S | F | OH |
| 1700a-l | Y*AC | S | O | O | O | S | S | OEt | OH |
| 1701a-l | Y*AC | S | S | O | O | S | S | OH | OH |
| 1702a-l | Y*AC | S | S | O | O | S | S | OMe | OH |
| 1703a-l | Y*AC | S | S | O | O | S | S | H | OH |
| 1704a-l | Y*AC | S | S | O | O | S | S | F | OH |
| 1705a-l | Y*AC | S | S | O | O | S | S | OEt | OH |
| 1706a-l | Y*AC | O | O | O | O | O | S | OH | OH |
| 1707a-l | Y*AC | O | O | O | O | O | S | OMe | OH |
| 1708a-l | Y*AC | O | O | O | O | O | S | H | OH |
| 1709a-l | Y*AC | O | O | O | O | O | S | F | OH |
| 1710a-l | Y*AC | O | O | O | O | O | S | OEt | OH |
| 1711a-l | Y*AC | S | O | O | O | O | S | OH | OH |
| 1712a-l | Y*AC | S | O | O | O | O | S | OMe | OH |
| 1713a-l | Y*AC | S | O | O | O | O | S | H | OH |
| 1714a-l | Y*AC | S | O | O | O | O | S | F | OH |
| 1715a-l | Y*AC | S | O | O | O | O | S | OEt | OH |
| 1716a-l | Y*AC | S | S | O | O | O | S | OH | OH |
| 1717a-l | Y*AC | S | S | O | O | O | S | OMe | OH |
| 1718a-l | Y*AC | S | S | O | O | O | S | H | OH |
| 1719a-l | Y*AC | S | S | O | O | O | S | F | OH |
| 1720a-l | Y*AC | S | S | O | O | O | S | OEt | OH |
| 1721a-l | Y*AC | O | O | S | O | S | O | OH | OH |
| 1722a-l | Y*AC | O | O | S | O | S | O | OMe | OH |
| 1723a-l | Y*AC | O | O | S | O | S | O | H | OH |
| 1724a-l | Y*AC | O | O | S | O | S | O | F | OH |
| 1725a-l | Y*AC | O | O | S | O | S | O | OEt | OH |
| 1726a-l | Y*AC | S | O | S | O | S | O | OH | OH |
| 1727a-l | Y*AC | S | O | S | O | S | O | OMe | OH |
| 1728a-l | Y*AC | S | O | S | O | S | O | H | OH |
| 1729a-l | Y*AC | S | O | S | O | S | O | F | OH |
| 1730a-l | Y*AC | S | O | S | O | S | O | OEt | OH |
| 1731a-l | Y*AC | S | S | S | O | S | O | OH | OH |
| 1732a-l | Y*AC | S | S | S | O | S | O | OMe | OH |
| 1733a-l | Y*AC | S | S | S | O | S | O | H | OH |
| 1734a-l | Y*AC | S | S | S | O | S | O | F | OH |
| 1735a-l | Y*AC | S | S | S | O | S | O | OEt | OH |
| 1736a-l | Y*AC | O | O | O | S | O | S | OH | OH |
| 1737a-l | Y*AC | O | O | O | S | O | S | OMe | OH |
| 1738a-l | Y*AC | O | O | O | S | O | S | H | OH |
| 1739a-l | Y*AC | O | O | O | S | O | S | F | OH |
| 1740a-l | Y*AC | O | O | O | S | O | S | OEt | OH |
| 1741a-l | Y*AC | S | O | O | S | O | S | OH | OH |
| 1742a-l | Y*AC | S | O | O | S | O | S | OMe | OH |
| 1743a-l | Y*AC | S | O | O | S | O | S | H | OH |
| 1744a-l | Y*AC | S | O | O | S | O | S | F | OH |
| 1745a-l | Y*AC | S | O | O | S | O | S | OEt | OH |
| 1746a-l | Y*AC | S | S | O | S | O | S | OH | OH |
| 1747a-l | Y*AC | S | S | O | S | O | S | OMe | OH |
| 1748a-l | Y*AC | S | S | O | S | O | S | H | OH |
| 1749a-l | Y*AC | S | S | O | S | O | S | F | OH |
| 1750a-l | Y*AC | S | S | O | S | O | S | OEt | OH |
| 1751a-l | Y*AC | O | O | O | O | O | O | OH | OMe |
| 1752a-l | Y*AC | O | O | O | O | O | O | OMe | OMe |
| 1753a-l | Y*AC | O | O | O | O | O | O | H | OMe |
| 1754a-l | Y*AC | O | O | O | O | O | O | F | OMe |
| 1755a-l | Y*AC | O | O | O | O | O | O | OEt | OMe |
| 1756a-l | Y*AC | S | O | O | O | O | O | OH | OMe |
| 1757a-l | Y*AC | S | O | O | O | O | O | OMe | OMe |
| 1758a-l | Y*AC | S | O | O | O | O | O | H | OMe |
| 1759a-l | Y*AC | S | O | O | O | O | O | F | OMe |
| 1760a-l | Y*AC | S | O | O | O | O | O | OEt | OMe |
| 1761a-l | Y*AC | S | S | O | O | O | O | OH | OMe |
| 1762a-l | Y*AC | S | S | O | O | O | O | OMe | OMe |
| 1763a-l | Y*AC | S | S | O | O | O | O | H | OMe |
| 1764a-l | Y*AC | S | S | O | O | O | O | F | OMe |
| 1765a-l | Y*AC | S | S | O | O | O | O | OEt | OMe |
| 1766a-l | Y*AC | O | O | S | O | O | O | OH | OMe |
| 1767a-l | Y*AC | O | O | S | O | O | O | OMe | OMe |
| 1768a-l | Y*AC | O | O | S | O | O | O | H | OMe |
| 1769a-l | Y*AC | O | O | S | O | O | O | F | OMe |
| 1770a-l | Y*AC | O | O | S | O | O | O | OEt | OMe |
| 1771a-l | Y*AC | S | O | S | O | O | O | OH | OMe |
| 1772a-l | Y*AC | S | O | S | O | O | O | OMe | OMe |
| 1773a-l | Y*AC | S | O | S | O | O | O | H | OMe |
| 1774a-l | Y*AC | S | O | S | O | O | O | F | OMe |
| 1775a-l | Y*AC | S | O | S | O | O | O | OEt | OMe |
| 1776a-l | Y*AC | S | S | S | O | O | O | OH | OMe |
| 1777a-l | Y*AC | S | S | S | O | O | O | OMe | OMe |
| 1778a-l | Y*AC | S | S | S | O | O | O | H | OMe |
| 1779a-l | Y*AC | S | S | S | O | O | O | F | OMe |
| 1780a-l | Y*AC | S | S | S | O | O | O | OEt | OMe |
| 1781a-l | Y*AC | O | O | S | S | O | O | OH | OMe |
| 1782a-l | Y*AC | O | O | S | S | O | O | OMe | OMe |
| 1783a-l | Y*AC | O | O | S | S | O | O | H | OMe |
| 1784a-l | Y*AC | O | O | S | S | O | O | F | OMe |
| 1785a-l | Y*AC | O | O | S | S | O | O | OEt | OMe |
| 1786a-l | Y*AC | S | O | S | S | O | O | OH | OMe |
| 1787a-l | Y*AC | S | O | S | S | O | O | OMe | OMe |
| 1788a-l | Y*AC | S | O | S | S | O | O | H | OMe |
| 1789a-l | Y*AC | S | O | S | S | O | O | F | OMe |
| 1790a-l | Y*AC | S | O | S | S | O | O | OEt | OMe |
| 1791a-l | Y*AC | S | S | S | S | O | O | OH | OMe |
| 1792a-l | Y*AC | S | S | S | S | O | O | OMe | OMe |
| 1793a-l | Y*AC | S | S | S | S | O | O | H | OMe |
| 1794a-l | Y*AC | S | S | S | S | O | O | F | OMe |
| 1795a-l | Y*AC | S | S | S | S | O | O | OEt | OMe |
| 1796a-l | Y*AC | O | O | S | S | S | O | OH | OMe |
| 1797a-l | Y*AC | O | O | S | S | S | O | OMe | OMe |
| 1798a-l | Y*AC | O | O | S | S | S | O | H | OMe |
| 1799a-l | Y*AC | O | O | S | S | S | O | F | OMe |
| 1800a-l | Y*AC | O | O | S | S | S | O | OEt | OMe |
| 1801a-l | Y*AC | S | O | S | S | S | O | OH | OMe |
| 1802a-l | Y*AC | S | O | S | S | S | O | OMe | OMe |
| 1803a-l | Y*AC | S | O | S | S | S | O | H | OMe |
| 1804a-l | Y*AC | S | O | S | S | S | O | F | OMe |
| 1805a-l | Y*AC | S | O | S | S | S | O | OEt | OMe |
| 1806a-l | Y*AC | S | S | S | S | S | O | OH | OMe |
| 1807a-l | Y*AC | S | S | S | S | S | O | OMe | OMe |
| 1808a-l | Y*AC | S | S | S | S | S | O | H | OMe |
| 1809a-l | Y*AC | S | S | S | S | S | O | F | OMe |
| 1810a-l | Y*AC | S | S | S | S | S | O | OEt | OMe |
| 1811a-l | Y*AC | O | O | S | S | S | S | OH | OMe |
| 1812a-l | Y*AC | O | O | S | S | S | S | OMe | OMe |
| 1813a-l | Y*AC | O | O | S | S | S | S | H | OMe |
| 1814a-l | Y*AC | O | O | S | S | S | S | F | OMe |
| 1815a-l | Y*AC | O | O | S | S | S | S | OEt | OMe |
| 1816a-l | Y*AC | S | O | S | S | S | S | OH | OMe |
| 1817a-l | Y*AC | S | O | S | S | S | S | OMe | OMe |
| 1818a-l | Y*AC | S | O | S | S | S | S | H | OMe |
| 1819a-l | Y*AC | S | O | S | S | S | S | F | OMe |
| 1820a-l | Y*AC | S | O | S | S | S | S | OEt | OMe |
| 1821a-l | Y*AC | S | S | S | S | S | S | OH | OMe |
| 1822a-l | Y*AC | S | S | S | S | S | S | OMe | OMe |
| 1823a-l | Y*AC | S | S | S | S | S | S | H | OMe |
| 1824a-l | Y*AC | S | S | S | S | S | S | F | OMe |
| 1825a-l | Y*AC | S | S | S | S | S | S | OEt | OMe |
| 1826a-l | Y*AC | O | O | O | S | S | S | OH | OMe |
| 1827a-l | Y*AC | O | O | O | S | S | S | OMe | OMe |
| 1828a-l | Y*AC | O | O | O | S | S | S | H | OMe |
| 1829a-l | Y*AC | O | O | O | S | S | S | F | OMe |
| 1830a-l | Y*AC | O | O | O | S | S | S | OEt | OMe |
| 1831a-l | Y*AC | S | O | O | S | S | S | OH | OMe |
| 1832a-l | Y*AC | S | O | O | S | S | S | OMe | OMe |
| 1833a-l | Y*AC | S | O | O | S | S | S | H | OMe |
| 1834a-l | Y*AC | S | O | O | S | S | S | F | OMe |
| 1835a-l | Y*AC | S | O | O | S | S | S | OEt | OMe |
| 1836a-l | Y*AC | S | S | O | S | S | S | OH | OMe |
| 1837a-l | Y*AC | S | S | O | S | S | S | OMe | OMe |
| 1838a-l | Y*AC | S | S | O | S | S | S | H | OMe |
| 1839a-l | Y*AC | S | S | O | S | S | S | F | OMe |
| 1840a-l | Y*AC | S | S | O | S | S | S | OEt | OMe |
| 1841a-l | Y*AC | O | O | O | O | S | S | OH | OMe |
| 1842a-l | Y*AC | O | O | O | O | S | S | OMe | OMe |
| 1843a-l | Y*AC | O | O | O | O | S | S | H | OMe |
| 1844a-l | Y*AC | O | O | O | O | S | S | F | OMe |
| 1845a-l | Y*AC | O | O | O | O | S | S | OEt | OMe |
| 1846a-l | Y*AC | S | O | O | O | S | S | OH | OMe |
| 1847a-l | Y*AC | S | O | O | O | S | S | OMe | OMe |
| 1848a-l | Y*AC | S | O | O | O | S | S | H | OMe |
| 1849a-l | Y*AC | S | O | O | O | S | S | F | OMe |
| 1850a-l | Y*AC | S | O | O | O | S | S | OEt | OMe |
| 1851a-l | Y*AC | S | S | O | O | S | S | OH | OMe |
| 1852a-l | Y*AC | S | S | O | O | S | S | OMe | OMe |
| 1853a-l | Y*AC | S | S | O | O | S | S | H | OMe |
| 1854a-l | Y*AC | S | S | O | O | S | S | F | OMe |
| 1855a-l | Y*AC | S | S | O | O | S | S | OEt | OMe |
| 1856a-l | Y*AC | O | O | O | O | O | S | OH | OMe |
| 1857a-l | Y*AC | O | O | O | O | O | S | OMe | OMe |
| 1858a-l | Y*AC | O | O | O | O | O | S | H | OMe |
| 1859a-l | Y*AC | O | O | O | O | O | S | F | OMe |
| 1860a-l | Y*AC | O | O | O | O | O | S | OEt | OMe |
| 1861a-l | Y*AC | S | O | O | O | O | S | OH | OMe |
| 1862a-l | Y*AC | S | O | O | O | O | S | OMe | OMe |
| 1863a-l | Y*AC | S | O | O | O | O | S | H | OMe |
| 1864a-l | Y*AC | S | O | O | O | O | S | F | OMe |
| 1865a-l | Y*AC | S | O | O | O | O | S | OEt | OMe |
| 1866a-l | Y*AC | S | S | O | O | O | S | OH | OMe |
| 1867a-l | Y*AC | S | S | O | O | O | S | OMe | OMe |
| 1868a-l | Y*AC | S | S | O | O | O | S | H | OMe |
| 1869a-l | Y*AC | S | S | O | O | O | S | F | OMe |
| 1870a-l | Y*AC | S | S | O | O | O | S | OEt | OMe |
| 1871a-l | Y*AC | O | O | S | O | S | O | OH | OMe |
| 1872a-l | Y*AC | O | O | S | O | S | O | OMe | OMe |
| 1873a-l | Y*AC | O | O | S | O | S | O | H | OMe |
| 1874a-l | Y*AC | O | O | S | O | S | O | F | OMe |
| 1875a-l | Y*AC | O | O | S | O | S | O | OEt | OMe |
| 1876a-l | Y*AC | S | O | S | O | S | O | OH | OMe |
| 1877a-l | Y*AC | S | O | S | O | S | O | OMe | OMe |
| 1878a-l | Y*AC | S | O | S | O | S | O | H | OMe |
| 1879a-l | Y*AC | S | O | S | O | S | O | F | OMe |
| 1880a-l | Y*AC | S | O | S | O | S | O | OEt | OMe |
| 1881a-l | Y*AC | S | S | S | O | S | O | OH | OMe |
| 1882a-l | Y*AC | S | S | S | O | S | O | OMe | OMe |
| 1883a-l | Y*AC | S | S | S | O | S | O | H | OMe |
| 1884a-l | Y*AC | S | S | S | O | S | O | F | OMe |
| 1885a-l | Y*AC | S | S | S | O | S | O | OEt | OMe |
| 1886a-l | Y*AC | O | O | O | S | O | S | OH | OMe |
| 1887a-l | Y*AC | O | O | O | S | O | S | OMe | OMe |
| 1888a-l | Y*AC | O | O | O | S | O | S | H | OMe |
| 1889a-l | Y*AC | O | O | O | S | O | S | F | OMe |
| 1890a-l | Y*AC | O | O | O | S | O | S | OEt | OMe |
| 1891a-l | Y*AC | S | O | O | S | O | S | OH | OMe |
| 1892a-l | Y*AC | S | O | O | S | O | S | OMe | OMe |
| 1893a-l | Y*AC | S | O | O | S | O | S | H | OMe |
| 1894a-l | Y*AC | S | O | O | S | O | S | F | OMe |
| 1895a-l | Y*AC | S | O | O | S | O | S | OEt | OMe |
| 1896a-l | Y*AC | S | S | O | S | O | S | OH | OMe |
| 1897a-l | Y*AC | S | S | O | S | O | S | OMe | OMe |
| 1898a-l | Y*AC | S | S | O | S | O | S | H | OMe |
| 1899a-l | Y*AC | S | S | O | S | O | S | F | OMe |
| 1900a-l | Y*AC | S | S | O | S | O | S | OEt | OMe |
| 1901a-l | Y*AU | O | O | O | O | O | O | OH | OH |
| 1902a-l | Y*AU | O | O | O | O | O | O | OMe | OH |
| 1903a-l | Y*AU | O | O | O | O | O | O | H | OH |
| 1904a-l | Y*AU | O | O | O | O | O | O | F | OH |
| 1905a-l | Y*AU | O | O | O | O | O | O | OEt | OH |
| 1906a-l | Y*AU | S | O | O | O | O | O | OH | OH |
| 1907a-l | Y*AU | S | O | O | O | O | O | OMe | OH |
| 1908a-l | Y*AU | S | O | O | O | O | O | H | OH |
| 1909a-l | Y*AU | S | O | O | O | O | O | F | OH |
| 1910a-l | Y*AU | S | O | O | O | O | O | OEt | OH |
| 1911a-l | Y*AU | S | S | O | O | O | O | OH | OH |
| 1912a-l | Y*AU | S | S | O | O | O | O | OMe | OH |
| 1913a-l | Y*AU | S | S | O | O | O | O | H | OH |
| 1914a-l | Y*AU | S | S | O | O | O | O | F | OH |
| 1915a-l | Y*AU | S | S | O | O | O | O | OEt | OH |
| 1916a-l | Y*AU | O | O | S | O | O | O | OH | OH |
| 1917a-l | Y*AU | O | O | S | O | O | O | OMe | OH |
| 1918a-l | Y*AU | O | O | S | O | O | O | H | OH |
| 1919a-l | Y*AU | O | O | S | O | O | O | F | OH |
| 1920a-l | Y*AU | O | O | S | O | O | O | OEt | OH |
| 1921a-l | Y*AU | S | O | S | O | O | O | OH | OH |
| 1922a-l | Y*AU | S | O | S | O | O | O | OMe | OH |
| 1923a-l | Y*AU | S | O | S | O | O | O | H | OH |
| 1924a-l | Y*AU | S | O | S | O | O | O | F | OH |
| 1925a-l | Y*AU | S | O | S | O | O | O | OEt | OH |
| 1926a-l | Y*AU | S | S | S | O | O | O | OH | OH |
| 1927a-l | Y*AU | S | S | S | O | O | O | OMe | OH |
| 1928a-l | Y*AU | S | S | S | O | O | O | H | OH |
| 1929a-l | Y*AU | S | S | S | O | O | O | F | OH |
| 1930a-l | Y*AU | S | S | S | O | O | O | OEt | OH |
| 1931a-l | Y*AU | O | O | S | S | O | O | OH | OH |
| 1932a-l | Y*AU | O | O | S | S | O | O | OMe | OH |
| 1933a-l | Y*AU | O | O | S | S | O | O | H | OH |
| 1934a-l | Y*AU | O | O | S | S | O | O | F | OH |
| 1935a-l | Y*AU | O | O | S | S | O | O | OEt | OH |
| 1936a-l | Y*AU | S | O | S | S | O | O | OH | OH |
| 1937a-l | Y*AU | S | O | S | S | O | O | OMe | OH |
| 1938a-l | Y*AU | S | O | S | S | O | O | H | OH |
| 1939a-l | Y*AU | S | O | S | S | O | O | F | OH |
| 1940a-l | Y*AU | S | O | S | S | O | O | OEt | OH |
| 1941a-l | Y*AU | S | S | S | S | O | O | OH | OH |
| 1942a-l | Y*AU | S | S | S | S | O | O | OMe | OH |
| 1943a-l | Y*AU | S | S | S | S | O | O | H | OH |
| 1944a-l | Y*AU | S | S | S | S | O | O | F | OH |
| 1945a-l | Y*AU | S | S | S | S | O | O | OEt | OH |
| 1946a-l | Y*AU | O | O | S | S | S | O | OH | OH |
| 1947a-l | Y*AU | O | O | S | S | S | O | OMe | OH |
| 1948a-l | Y*AU | O | O | S | S | S | O | H | OH |
| 1949a-l | Y*AU | O | O | S | S | S | O | F | OH |
| 1950a-l | Y*AU | O | O | S | S | S | O | OEt | OH |
| 1951a-l | Y*AU | S | O | S | S | S | O | OH | OH |
| 1952a-l | Y*AU | S | O | S | S | S | O | OMe | OH |
| 1953a-l | Y*AU | S | O | S | S | S | O | H | OH |
| 1954a-l | Y*AU | S | O | S | S | S | O | F | OH |
| 1955a-l | Y*AU | S | O | S | S | S | O | OEt | OH |
| 1956a-l | Y*AU | S | S | S | S | S | O | OH | OH |
| 1957a-l | Y*AU | S | S | S | S | S | O | OMe | OH |
| 1958a-l | Y*AU | S | S | S | S | S | O | H | OH |
| 1959a-l | Y*AU | S | S | S | S | S | O | F | OH |
| 1960a-l | Y*AU | S | S | S | S | S | O | OEt | OH |
| 1961a-l | Y*AU | O | O | S | S | S | S | OH | OH |
| 1962a-l | Y*AU | O | O | S | S | S | S | OMe | OH |
| 1963a-l | Y*AU | O | O | S | S | S | S | H | OH |
| 1964a-l | Y*AU | O | O | S | S | S | S | F | OH |
| 1965a-l | Y*AU | O | O | S | S | S | S | OEt | OH |
| 1966a-l | Y*AU | S | O | S | S | S | S | OH | OH |
| 1967a-l | Y*AU | S | O | S | S | S | S | OMe | OH |
| 1968a-l | Y*AU | S | O | S | S | S | S | H | OH |
| 1969a-l | Y*AU | S | O | S | S | S | S | F | OH |
| 1970a-l | Y*AU | S | O | S | S | S | S | OEt | OH |
| 1971a-l | Y*AU | S | S | S | S | S | S | OH | OH |
| 1972a-l | Y*AU | S | S | S | S | S | S | OMe | OH |
| 1973a-l | Y*AU | S | S | S | S | S | S | H | OH |
| 1974a-l | Y*AU | S | S | S | S | S | S | F | OH |
| 1975a-l | Y*AU | S | S | S | S | S | S | OEt | OH |
| 1976a-l | Y*AU | O | O | O | S | S | S | OH | OH |
| 1977a-l | Y*AU | O | O | O | S | S | S | OMe | OH |
| 1978a-l | Y*AU | O | O | O | S | S | S | H | OH |
| 1979a-l | Y*AU | O | O | O | S | S | S | F | OH |
| 1980a-l | Y*AU | O | O | O | S | S | S | OEt | OH |
| 1981a-l | Y*AU | S | O | O | S | S | S | OH | OH |
| 1982a-l | Y*AU | S | O | O | S | S | S | OMe | OH |
| 1983a-l | Y*AU | S | O | O | S | S | S | H | OH |
| 1984a-l | Y*AU | S | O | O | S | S | S | F | OH |
| 1985a-l | Y*AU | S | O | O | S | S | S | OEt | OH |
| 1986a-l | Y*AU | S | S | O | S | S | S | OH | OH |
| 1987a-l | Y*AU | S | S | O | S | S | S | OMe | OH |
| 1988a-l | Y*AU | S | S | O | S | S | S | H | OH |
| 1989a-l | Y*AU | S | S | O | S | S | S | F | OH |
| 1990a-l | Y*AU | S | S | O | S | S | S | OEt | OH |
| 1991a-l | Y*AU | O | O | O | O | S | S | OH | OH |
| 1992a-l | Y*AU | O | O | O | O | S | S | OMe | OH |
| 1993a-l | Y*AU | O | O | O | O | S | S | H | OH |
| 1994a-l | Y*AU | O | O | O | O | S | S | F | OH |
| 1995a-l | Y*AU | O | O | O | O | S | S | OEt | OH |
| 1996a-l | Y*AU | S | O | O | O | S | S | OH | OH |
| 1997a-l | Y*AU | S | O | O | O | S | S | OMe | OH |
| 1998a-l | Y*AU | S | O | O | O | S | S | H | OH |
| 1999a-l | Y*AU | S | O | O | O | S | S | F | OH |
| 2000a-l | Y*AU | S | O | O | O | S | S | OEt | OH |
| 2001a-l | Y*AU | S | S | O | O | S | S | OH | OH |
| 2002a-l | Y*AU | S | S | O | O | S | S | OMe | OH |
| 2003a-l | Y*AU | S | S | O | O | S | S | H | OH |
| 2004a-l | Y*AU | S | S | O | O | S | S | F | OH |
| 2005a-l | Y*AU | S | S | O | O | S | S | OEt | OH |
| 2006a-l | Y*AU | O | O | O | O | O | S | OH | OH |
| 2007a-l | Y*AU | O | O | O | O | O | S | OMe | OH |
| 2008a-l | Y*AU | O | O | O | O | O | S | H | OH |
| 2009a-l | Y*AU | O | O | O | O | O | S | F | OH |
| 2010a-l | Y*AU | O | O | O | O | O | S | OEt | OH |
| 2011a-l | Y*AU | S | O | O | O | O | S | OH | OH |
| 2012a-l | Y*AU | S | O | O | O | O | S | OMe | OH |
| 2013a-l | Y*AU | S | O | O | O | O | S | H | OH |
| 2014a-l | Y*AU | S | O | O | O | O | S | F | OH |
| 2015a-l | Y*AU | S | O | O | O | O | S | OEt | OH |
| 2016a-l | Y*AU | S | S | O | O | O | S | OH | OH |
| 2017a-l | Y*AU | S | S | O | O | O | S | OMe | OH |
| 2018a-l | Y*AU | S | S | O | O | O | S | H | OH |
| 2019a-l | Y*AU | S | S | O | O | O | S | F | OH |
| 2020a-l | Y*AU | S | S | O | O | O | S | OEt | OH |
| 2021a-l | Y*AU | O | O | S | O | S | O | OH | OH |
| 2022a-l | Y*AU | O | O | S | O | S | O | OMe | OH |
| 2023a-l | Y*AU | O | O | S | O | S | O | H | OH |
| 2024a-l | Y*AU | O | O | S | O | S | O | F | OH |
| 2025a-l | Y*AU | O | O | S | O | S | O | OEt | OH |
| 2026a-l | Y*AU | S | O | S | O | S | O | OH | OH |
| 2027a-l | Y*AU | S | O | S | O | S | O | OMe | OH |
| 2028a-l | Y*AU | S | O | S | O | S | O | H | OH |
| 2029a-l | Y*AU | S | O | S | O | S | O | F | OH |
| 2030a-l | Y*AU | S | O | S | O | S | O | OEt | OH |
| 2031a-l | Y*AU | S | S | S | O | S | O | OH | OH |
| 2032a-l | Y*AU | S | S | S | O | S | O | OMe | OH |
| 2033a-l | Y*AU | S | S | S | O | S | O | H | OH |
| 2034a-l | Y*AU | S | S | S | O | S | O | F | OH |
| 2035a-l | Y*AU | S | S | S | O | S | O | OEt | OH |
| 2036a-l | Y*AU | O | O | O | S | O | S | OH | OH |
| 2037a-l | Y*AU | O | O | O | S | O | S | OMe | OH |
| 2038a-l | Y*AU | O | O | O | S | O | S | H | OH |
| 2039a-l | Y*AU | O | O | O | S | O | S | F | OH |
| 2040a-l | Y*AU | O | O | O | S | O | S | OEt | OH |
| 2041a-l | Y*AU | S | O | O | S | O | S | OH | OH |
| 2042a-l | Y*AU | S | O | O | S | O | S | OMe | OH |
| 2043a-l | Y*AU | S | O | O | S | O | S | H | OH |
| 2044a-l | Y*AU | S | O | O | S | O | S | F | OH |
| 2045a-l | Y*AU | S | O | O | S | O | S | OEt | OH |
| 2046a-l | Y*AU | S | S | O | S | O | S | OH | OH |
| 2047a-l | Y*AU | S | S | O | S | O | S | OMe | OH |
| 2048a-l | Y*AU | S | S | O | S | O | S | H | OH |
| 2049a-l | Y*AU | S | S | O | S | O | S | F | OH |
| 2050a-l | Y*AU | S | S | O | S | O | S | OEt | OH |
| 2051a-l | Y*AU | O | O | O | O | O | O | OH | OMe |
| 2052a-l | Y*AU | O | O | O | O | O | O | OMe | OMe |
| 2053a-l | Y*AU | O | O | O | O | O | O | H | OMe |
| 2054a-l | Y*AU | O | O | O | O | O | O | F | OMe |
| 2055a-l | Y*AU | O | O | O | O | O | O | OEt | OMe |
| 2056a-l | Y*AU | S | O | O | O | O | O | OH | OMe |
| 2057a-l | Y*AU | S | O | O | O | O | O | OMe | OMe |
| 2058a-l | Y*AU | S | O | O | O | O | O | H | OMe |
| 2059a-l | Y*AU | S | O | O | O | O | O | F | OMe |
| 2060a-l | Y*AU | S | O | O | O | O | O | OEt | OMe |
| 2061a-l | Y*AU | S | S | O | O | O | O | OH | OMe |
| 2062a-l | Y*AU | S | S | O | O | O | O | OMe | OMe |
| 2063a-l | Y*AU | S | S | O | O | O | O | H | OMe |
| 2064a-l | Y*AU | S | S | O | O | O | O | F | OMe |
| 2065a-l | Y*AU | S | S | O | O | O | O | OEt | OMe |
| 2066a-l | Y*AU | O | O | S | O | O | O | OH | OMe |
| 2067a-l | Y*AU | O | O | S | O | O | O | OMe | OMe |
| 2068a-l | Y*AU | O | O | S | O | O | O | H | OMe |
| 2069a-l | Y*AU | O | O | S | O | O | O | F | OMe |
| 2070a-l | Y*AU | O | O | S | O | O | O | OEt | OMe |
| 2071a-l | Y*AU | S | O | S | O | O | O | OH | OMe |
| 2072a-l | Y*AU | S | O | S | O | O | O | OMe | OMe |
| 2073a-l | Y*AU | S | O | S | O | O | O | H | OMe |
| 2074a-l | Y*AU | S | O | S | O | O | O | F | OMe |
| 2075a-l | Y*AU | S | O | S | O | O | O | OEt | OMe |
| 2076a-l | Y*AU | S | S | S | O | O | O | OH | OMe |
| 2077a-l | Y*AU | S | S | S | O | O | O | OMe | OMe |
| 2078a-l | Y*AU | S | S | S | O | O | O | H | OMe |
| 2079a-l | Y*AU | S | S | S | O | O | O | F | OMe |
| 2080a-l | Y*AU | S | S | S | O | O | O | OEt | OMe |
| 2081a-l | Y*AU | O | O | S | S | O | O | OH | OMe |
| 2082a-l | Y*AU | O | O | S | S | O | O | OMe | OMe |
| 2083a-l | Y*AU | O | O | S | S | O | O | H | OMe |
| 2084a-l | Y*AU | O | O | S | S | O | O | F | OMe |
| 2085a-l | Y*AU | O | O | S | S | O | O | OEt | OMe |
| 2086a-l | Y*AU | S | O | S | S | O | O | OH | OMe |
| 2087a-l | Y*AU | S | O | S | S | O | O | OMe | OMe |
| 2088a-l | Y*AU | S | O | S | S | O | O | H | OMe |
| 2089a-l | Y*AU | S | O | S | S | O | O | F | OMe |
| 2090a-l | Y*AU | S | O | S | S | O | O | OEt | OMe |
| 2091a-l | Y*AU | S | S | S | S | O | O | OH | OMe |
| 2092a-l | Y*AU | S | S | S | S | O | O | OMe | OMe |
| 2093a-l | Y*AU | S | S | S | S | O | O | H | OMe |
| 2094a-l | Y*AU | S | S | S | S | O | O | F | OMe |
| 2095a-l | Y*AU | S | S | S | S | O | O | OEt | OMe |
| 2096a-l | Y*AU | O | O | S | S | S | O | OH | OMe |
| 2097a-l | Y*AU | O | O | S | S | S | O | OMe | OMe |
| 2098a-l | Y*AU | O | O | S | S | S | O | H | OMe |
| 2099a-l | Y*AU | O | O | S | S | S | O | F | OMe |
| 2100a-l | Y*AU | O | O | S | S | S | O | OEt | OMe |
| 2101a-l | Y*AU | S | O | S | S | S | O | OH | OMe |
| 2102a-l | Y*AU | S | O | S | S | S | O | OMe | OMe |
| 2103a-l | Y*AU | S | O | S | S | S | O | H | OMe |
| 2104a-l | Y*AU | S | O | S | S | S | O | F | OMe |
| 2105a-l | Y*AU | S | O | S | S | S | O | OEt | OMe |
| 2106a-l | Y*AU | S | S | S | S | S | O | OH | OMe |
| 2107a-l | Y*AU | S | S | S | S | S | O | OMe | OMe |
| 2108a-l | Y*AU | S | S | S | S | S | O | H | OMe |
| 2109a-l | Y*AU | S | S | S | S | S | O | F | OMe |
| 2110a-l | Y*AU | S | S | S | S | S | O | OEt | OMe |
| 2111a-l | Y*AU | O | O | S | S | S | S | OH | OMe |
| 2112a-l | Y*AU | O | O | S | S | S | S | OMe | OMe |
| 2113a-l | Y*AU | O | O | S | S | S | S | H | OMe |
| 2114a-l | Y*AU | O | O | S | S | S | S | F | OMe |
| 2115a-l | Y*AU | O | O | S | S | S | S | OEt | OMe |
| 2116a-l | Y*AU | S | O | S | S | S | S | OH | OMe |
| 2117a-l | Y*AU | S | O | S | S | S | S | OMe | OMe |
| 2118a-l | Y*AU | S | O | S | S | S | S | H | OMe |
| 2119a-l | Y*AU | S | O | S | S | S | S | F | OMe |
| 2120a-l | Y*AU | S | O | S | S | S | S | OEt | OMe |
| 2121a-l | Y*AU | S | S | S | S | S | S | OH | OMe |
| 2122a-l | Y*AU | S | S | S | S | S | S | OMe | OMe |
| 2123a-l | Y*AU | S | S | S | S | S | S | H | OMe |
| 2124a-l | Y*AU | S | S | S | S | S | S | F | OMe |
| 2125a-l | Y*AU | S | S | S | S | S | S | OEt | OMe |
| 2126a-l | Y*AU | O | O | O | S | S | S | OH | OMe |
| 2127a-l | Y*AU | O | O | O | S | S | S | OMe | OMe |
| 2128a-l | Y*AU | O | O | O | S | S | S | H | OMe |
| 2129a-l | Y*AU | O | O | O | S | S | S | F | OMe |
| 2130a-l | Y*AU | O | O | O | S | S | S | OEt | OMe |
| 2131a-l | Y*AU | S | O | O | S | S | S | OH | OMe |
| 2132a-l | Y*AU | S | O | O | S | S | S | OMe | OMe |
| 2133a-l | Y*AU | S | O | O | S | S | S | H | OMe |
| 2134a-l | Y*AU | S | O | O | S | S | S | F | OMe |
| 2135a-l | Y*AU | S | O | O | S | S | S | OEt | OMe |
| 2136a-l | Y*AU | S | S | O | S | S | S | OH | OMe |
| 2137a-l | Y*AU | S | S | O | S | S | S | OMe | OMe |
| 2138a-l | Y*AU | S | S | O | S | S | S | H | OMe |
| 2139a-l | Y*AU | S | S | O | S | S | S | F | OMe |
| 2140a-l | Y*AU | S | S | O | S | S | S | OEt | OMe |
| 2141a-l | Y*AU | O | O | O | O | S | S | OH | OMe |
| 2142a-l | Y*AU | O | O | O | O | S | S | OMe | OMe |
| 2143a-l | Y*AU | O | O | O | O | S | S | H | OMe |
| 2144a-l | Y*AU | O | O | O | O | S | S | F | OMe |
| 2145a-l | Y*AU | O | O | O | O | S | S | OEt | OMe |
| 2146a-l | Y*AU | S | O | O | O | S | S | OH | OMe |
| 2147a-l | Y*AU | S | O | O | O | S | S | OMe | OMe |
| 2148a-l | Y*AU | S | O | O | O | S | S | H | OMe |
| 2149a-l | Y*AU | S | O | O | O | S | S | F | OMe |
| 2150a-l | Y*AU | S | O | O | O | S | S | OEt | OMe |
| 2151a-l | Y*AU | S | S | O | O | S | S | OH | OMe |
| 2152a-l | Y*AU | S | S | O | O | S | S | OMe | OMe |
| 2153a-l | Y*AU | S | S | O | O | S | S | H | OMe |
| 2154a-l | Y*AU | S | S | O | O | S | S | F | OMe |
| 2155a-l | Y*AU | S | S | O | O | S | S | OEt | OMe |
| 2156a-l | Y*AU | O | O | O | O | O | S | OH | OMe |
| 2157a-l | Y*AU | O | O | O | O | O | S | OMe | OMe |
| 2158a-l | Y*AU | O | O | O | O | O | S | H | OMe |
| 2159a-l | Y*AU | O | O | O | O | O | S | F | OMe |
| 2160a-l | Y*AU | O | O | O | O | O | S | OEt | OMe |
| 2161a-l | Y*AU | S | O | O | O | O | S | OH | OMe |
| 2162a-l | Y*AU | S | O | O | O | O | S | OMe | OMe |
| 2163a-l | Y*AU | S | O | O | O | O | S | H | OMe |
| 2164a-l | Y*AU | S | O | O | O | O | S | F | OMe |
| 2165a-l | Y*AU | S | O | O | O | O | S | OEt | OMe |
| 2166a-l | Y*AU | S | S | O | O | O | S | OH | OMe |
| 2167a-l | Y*AU | S | S | O | O | O | S | OMe | OMe |
| 2168a-l | Y*AU | S | S | O | O | O | S | H | OMe |
| 2169a-l | Y*AU | S | S | O | O | O | S | F | OMe |
| 2170a-l | Y*AU | S | S | O | O | O | S | OEt | OMe |
| 2171a-l | Y*AU | O | O | S | O | S | O | OH | OMe |
| 2172a-l | Y*AU | O | O | S | O | S | O | OMe | OMe |
| 2173a-l | Y*AU | O | O | S | O | S | O | H | OMe |
| 2174a-l | Y*AU | O | O | S | O | S | O | F | OMe |
| 2175a-l | Y*AU | O | O | S | O | S | O | OEt | OMe |
| 2176a-l | Y*AU | S | O | S | O | S | O | OH | OMe |
| 2177a-l | Y*AU | S | O | S | O | S | O | OMe | OMe |
| 2178a-l | Y*AU | S | O | S | O | S | O | H | OMe |
| 2179a-l | Y*AU | S | O | S | O | S | O | F | OMe |
| 2180a-l | Y*AU | S | O | S | O | S | O | OEt | OMe |
| 2181a-l | Y*AU | S | S | S | O | S | O | OH | OMe |
| 2182a-l | Y*AU | S | S | S | O | S | O | OMe | OMe |
| 2183a-l | Y*AU | S | S | S | O | S | O | H | OMe |
| 2184a-l | Y*AU | S | S | S | O | S | O | F | OMe |
| 2185a-l | Y*AU | S | S | S | O | S | O | OEt | OMe |
| 2186a-l | Y*AU | O | O | O | S | O | S | OH | OMe |
| 2187a-l | Y*AU | O | O | O | S | O | S | OMe | OMe |
| 2188a-l | Y*AU | O | O | O | S | O | S | H | OMe |
| 2189a-l | Y*AU | O | O | O | S | O | S | F | OMe |
| 2190a-l | Y*AU | O | O | O | S | O | S | OEt | OMe |
| 2191a-l | Y*AU | S | O | O | S | O | S | OH | OMe |
| 2192a-l | Y*AU | S | O | O | S | O | S | OMe | OMe |
| 2193a-l | Y*AU | S | O | O | S | O | S | H | OMe |
| 2194a-l | Y*AU | S | O | O | S | O | S | F | OMe |
| 2195a-l | Y*AU | S | O | O | S | O | S | OEt | OMe |
| 2196a-l | Y*AU | S | S | O | S | O | S | OH | OMe |
| 2197a-l | Y*AU | S | S | O | S | O | S | OMe | OMe |
| 2198a-l | Y*AU | S | S | O | S | O | S | H | OMe |
| 2199a-l | Y*AU | S | S | O | S | O | S | F | OMe |
| 2200a-l | Y*AU | S | S | O | S | O | S | OEt | OMe |
| 2201a-l | Y*AI | O | O | O | O | O | O | OH | OH |
| 2202a-l | Y*AI | O | O | O | O | O | O | OMe | OH |
| 2203a-l | Y*AI | O | O | O | O | O | O | H | OH |
| 2204a-l | Y*AI | O | O | O | O | O | O | F | OH |
| 2205a-l | Y*AI | O | O | O | O | O | O | OEt | OH |
| 2206a-l | Y*AI | S | O | O | O | O | O | OH | OH |
| 2207a-l | Y*AI | S | O | O | O | O | O | OMe | OH |
| 2208a-l | Y*AI | S | O | O | O | O | O | H | OH |
| 2209a-l | Y*AI | S | O | O | O | O | O | F | OH |
| 2210a-l | Y*AI | S | O | O | O | O | O | OEt | OH |
| 2211a-l | Y*AI | S | S | O | O | O | O | OH | OH |
| 2212a-l | Y*AI | S | S | O | O | O | O | OMe | OH |
| 2213a-l | Y*AI | S | S | O | O | O | O | H | OH |
| 2214a-l | Y*AI | S | S | O | O | O | O | F | OH |
| 2215a-l | Y*AI | S | S | O | O | O | O | OEt | OH |
| 2216a-l | Y*AI | O | O | S | O | O | O | OH | OH |
| 2217a-l | Y*AI | O | O | S | O | O | O | OMe | OH |
| 2218a-l | Y*AI | O | O | S | O | O | O | H | OH |
| 2219a-l | Y*AI | O | O | S | O | O | O | F | OH |
| 2220a-l | Y*AI | O | O | S | O | O | O | OEt | OH |
| 2221a-l | Y*AI | S | O | S | O | O | O | OH | OH |
| 2222a-l | Y*AI | S | O | S | O | O | O | OMe | OH |
| 2223a-l | Y*AI | S | O | S | O | O | O | H | OH |
| 2224a-l | Y*AI | S | O | S | O | O | O | F | OH |
| 2225a-l | Y*AI | S | O | S | O | O | O | OEt | OH |
| 2226a-l | Y*AI | S | S | S | O | O | O | OH | OH |
| 2227a-l | Y*AI | S | S | S | O | O | O | OMe | OH |
| 2228a-l | Y*AI | S | S | S | O | O | O | H | OH |
| 2229a-l | Y*AI | S | S | S | O | O | O | F | OH |
| 2230a-l | Y*AI | S | S | S | O | O | O | OEt | OH |
| 2231a-l | Y*AI | O | O | S | S | O | O | OH | OH |
| 2232a-l | Y*AI | O | O | S | S | O | O | OMe | OH |
| 2233a-l | Y*AI | O | O | S | S | O | O | H | OH |
| 2234a-l | Y*AI | O | O | S | S | O | O | F | OH |
| 2235a-l | Y*AI | O | O | S | S | O | O | OEt | OH |
| 2236a-l | Y*AI | S | O | S | S | O | O | OH | OH |
| 2237a-l | Y*AI | S | O | S | S | O | O | OMe | OH |
| 2238a-l | Y*AI | S | O | S | S | O | O | H | OH |
| 2239a-l | Y*AI | S | O | S | S | O | O | F | OH |
| 2240a-l | Y*AI | S | O | S | S | O | O | OEt | OH |
| 2241a-l | Y*AI | S | S | S | S | O | O | OH | OH |
| 2242a-l | Y*AI | S | S | S | S | O | O | OMe | OH |
| 2243a-l | Y*AI | S | S | S | S | O | O | H | OH |
| 2244a-l | Y*AI | S | S | S | S | O | O | F | OH |
| 2245a-l | Y*AI | S | S | S | S | O | O | OEt | OH |
| 2246a-l | Y*AI | O | O | S | S | S | O | OH | OH |
| 2247a-l | Y*AI | O | O | S | S | S | O | OMe | OH |
| 2248a-l | Y*AI | O | O | S | S | S | O | H | OH |
| 2249a-l | Y*AI | O | O | S | S | S | O | F | OH |
| 2250a-l | Y*AI | O | O | S | S | S | O | OEt | OH |
| 2251a-l | Y*AI | S | O | S | S | S | O | OH | OH |
| 2252a-l | Y*AI | S | O | S | S | S | O | OMe | OH |
| 2253a-l | Y*AI | S | O | S | S | S | O | H | OH |
| 2254a-l | Y*AI | S | O | S | S | S | O | F | OH |
| 2255a-l | Y*AI | S | O | S | S | S | O | OEt | OH |
| 2256a-l | Y*AI | S | S | S | S | S | O | OH | OH |
| 2257a-l | Y*AI | S | S | S | S | S | O | OMe | OH |
| 2258a-l | Y*AI | S | S | S | S | S | O | H | OH |
| 2259a-l | Y*AI | S | S | S | S | S | O | F | OH |
| 2260a-l | Y*AI | S | S | S | S | S | O | OEt | OH |
| 2261a-l | Y*AI | O | O | S | S | S | S | OH | OH |
| 2262a-l | Y*AI | O | O | S | S | S | S | OMe | OH |
| 2263a-l | Y*AI | O | O | S | S | S | S | H | OH |
| 2264a-l | Y*AI | O | O | S | S | S | S | F | OH |
| 2265a-l | Y*AI | O | O | S | S | S | S | OEt | OH |
| 2266a-l | Y*AI | S | O | S | S | S | S | OH | OH |
| 2267a-l | Y*AI | S | O | S | S | S | S | OMe | OH |
| 2268a-l | Y*AI | S | O | S | S | S | S | H | OH |
| 2269a-l | Y*AI | S | O | S | S | S | S | F | OH |
| 2270a-l | Y*AI | S | O | S | S | S | S | OEt | OH |
| 2271a-l | Y*AI | S | S | S | S | S | S | OH | OH |
| 2272a-l | Y*AI | S | S | S | S | S | S | OMe | OH |
| 2273a-l | Y*AI | S | S | S | S | S | S | H | OH |
| 2274a-l | Y*AI | S | S | S | S | S | S | F | OH |
| 2275a-l | Y*AI | S | S | S | S | S | S | OEt | OH |
| 2276a-l | Y*AI | O | O | O | S | S | S | OH | OH |
| 2277a-l | Y*AI | O | O | O | S | S | S | OMe | OH |
| 2278a-l | Y*AI | O | O | O | S | S | S | H | OH |
| 2279a-l | Y*AI | O | O | O | S | S | S | F | OH |
| 2280a-l | Y*AI | O | O | O | S | S | S | OEt | OH |
| 2281a-l | Y*AI | S | O | O | S | S | S | OH | OH |
| 2282a-l | Y*AI | S | O | O | S | S | S | OMe | OH |
| 2283a-l | Y*AI | S | O | O | S | S | S | H | OH |
| 2284a-l | Y*AI | S | O | O | S | S | S | F | OH |
| 2285a-l | Y*AI | S | O | O | S | S | S | OEt | OH |
| 2286a-l | Y*AI | S | S | O | S | S | S | OH | OH |
| 2287a-l | Y*AI | S | S | O | S | S | S | OMe | OH |
| 2288a-l | Y*AI | S | S | O | S | S | S | H | OH |
| 2289a-l | Y*AI | S | S | O | S | S | S | F | OH |
| 2290a-l | Y*AI | S | S | O | S | S | S | OEt | OH |
| 2291a-l | Y*AI | O | O | O | O | S | S | OH | OH |
| 2292a-l | Y*AI | O | O | O | O | S | S | OMe | OH |
| 2293a-l | Y*AI | O | O | O | O | S | S | H | OH |
| 2294a-l | Y*AI | O | O | O | O | S | S | F | OH |
| 2295a-l | Y*AI | O | O | O | O | S | S | OEt | OH |
| 2296a-l | Y*AI | S | O | O | O | S | S | OH | OH |
| 2297a-l | Y*AI | S | O | O | O | S | S | OMe | OH |
| 2298a-l | Y*AI | S | O | O | O | S | S | H | OH |
| 2299a-l | Y*AI | S | O | O | O | S | S | F | OH |
| 2300a-l | Y*AI | S | O | O | O | S | S | OEt | OH |
| 2301a-l | Y*AI | S | S | O | O | S | S | OH | OH |
| 2302a-l | Y*AI | S | S | O | O | S | S | OMe | OH |
| 2303a-l | Y*AI | S | S | O | O | S | S | H | OH |
| 2304a-l | Y*AI | S | S | O | O | S | S | F | OH |
| 2305a-l | Y*AI | S | S | O | O | S | S | OEt | OH |
| 2306a-l | Y*AI | O | O | O | O | O | S | OH | OH |
| 2307a-l | Y*AI | O | O | O | O | O | S | OMe | OH |
| 2308a-l | Y*AI | O | O | O | O | O | S | H | OH |
| 2309a-l | Y*AI | O | O | O | O | O | S | F | OH |
| 2310a-l | Y*AI | O | O | O | O | O | S | OEt | OH |
| 2311a-l | Y*AI | S | O | O | O | O | S | OH | OH |
| 2312a-l | Y*AI | S | O | O | O | O | S | OMe | OH |
| 2313a-l | Y*AI | S | O | O | O | O | S | H | OH |
| 2314a-l | Y*AI | S | O | O | O | O | S | F | OH |
| 2315a-l | Y*AI | S | O | O | O | O | S | OEt | OH |
| 2316a-l | Y*AI | S | S | O | O | O | S | OH | OH |
| 2317a-l | Y*AI | S | S | O | O | O | S | OMe | OH |
| 2318a-l | Y*AI | S | S | O | O | O | S | H | OH |
| 2319a-l | Y*AI | S | S | O | O | O | S | F | OH |
| 2320a-l | Y*AI | S | S | O | O | O | S | OEt | OH |
| 2321a-l | Y*AI | O | O | S | O | S | O | OH | OH |
| 2322a-l | Y*AI | O | O | S | O | S | O | OMe | OH |
| 2323a-l | Y*AI | O | O | S | O | S | O | H | OH |
| 2324a-l | Y*AI | O | O | S | O | S | O | F | OH |
| 2325a-l | Y*AI | O | O | S | O | S | O | OEt | OH |
| 2326a-l | Y*AI | S | O | S | O | S | O | OH | OH |
| 2327a-l | Y*AI | S | O | S | O | S | O | OMe | OH |
| 2328a-l | Y*AI | S | O | S | O | S | O | H | OH |
| 2329a-l | Y*AI | S | O | S | O | S | O | F | OH |
| 2330a-l | Y*AI | S | O | S | O | S | O | OEt | OH |
| 2331a-l | Y*AI | S | S | S | O | S | O | OH | OH |
| 2332a-l | Y*AI | S | S | S | O | S | O | OMe | OH |
| 2333a-l | Y*AI | S | S | S | O | S | O | H | OH |
| 2334a-l | Y*AI | S | S | S | O | S | O | F | OH |
| 2335a-l | Y*AI | S | S | S | O | S | O | OEt | OH |
| 2336a-l | Y*AI | O | O | O | S | O | S | OH | OH |
| 2337a-l | Y*AI | O | O | O | S | O | S | OMe | OH |
| 2338a-l | Y*AI | O | O | O | S | O | S | H | OH |
| 2339a-l | Y*AI | O | O | O | S | O | S | F | OH |
| 2340a-l | Y*AI | O | O | O | S | O | S | OEt | OH |
| 2341a-l | Y*AI | S | O | O | S | O | S | OH | OH |
| 2342a-l | Y*AI | S | O | O | S | O | S | OMe | OH |
| 2343a-l | Y*AI | S | O | O | S | O | S | H | OH |
| 2344a-l | Y*AI | S | O | O | S | O | S | F | OH |
| 2345a-l | Y*AI | S | O | O | S | O | S | OEt | OH |
| 2346a-l | Y*AI | S | S | O | S | O | S | OH | OH |
| 2347a-l | Y*AI | S | S | O | S | O | S | OMe | OH |
| 2348a-l | Y*AI | S | S | O | S | O | S | H | OH |
| 2349a-l | Y*AI | S | S | C | S | O | S | F | OH |
| 2350a-l | Y*AI | S | S | O | S | O | S | OEt | OH |
| 2351a-l | Y*AI | O | O | O | O | O | O | OH | OMe |
| 2352a-l | Y*AI | O | O | O | O | O | O | OMe | OMe |
| 2353a-l | Y*AI | O | O | O | O | O | O | H | OMe |
| 2354a-l | Y*AI | O | O | O | O | O | O | F | OMe |
| 2355a-l | Y*AI | O | O | O | O | O | O | OEt | OMe |
| 2356a-l | Y*AI | S | O | O | O | O | O | OH | OMe |
| 2357a-l | Y*AI | S | O | O | O | O | O | OMe | OMe |
| 2358a-l | Y*AI | S | O | O | O | O | O | H | OMe |
| 2359a-l | Y*AI | S | O | O | O | O | O | F | OMe |
| 2360a-l | Y*AI | S | O | O | O | O | O | OEt | OMe |
| 2361a-l | Y*AI | S | S | O | O | O | O | OH | OMe |
| 2362a-l | Y*AI | S | S | O | O | O | O | OMe | OMe |
| 2363a-l | Y*AI | S | S | O | O | O | O | H | OMe |
| 2364a-l | Y*AI | S | S | O | O | O | O | F | OMe |
| 2365a-l | Y*AI | S | S | O | O | O | O | OEt | OMe |
| 2366a-l | Y*AI | O | O | S | O | O | O | OH | OMe |
| 2367a-l | Y*AI | O | O | S | O | O | O | OMe | OMe |
| 2368a-l | Y*AI | O | O | S | O | O | O | H | OMe |
| 2369a-l | Y*AI | O | O | S | O | O | O | F | OMe |
| 2370a-l | Y*AI | O | O | S | O | O | O | OEt | OMe |
| 2371a-l | Y*AI | S | O | S | O | O | O | OH | OMe |
| 2372a-l | Y*AI | S | O | S | O | O | O | OMe | OMe |
| 2373a-l | Y*AI | S | O | S | O | O | O | H | OMe |
| 2374a-l | Y*AI | S | O | S | O | O | O | F | OMe |
| 2375a-l | Y*AI | S | O | S | O | O | O | OEt | OMe |
| 2376a-l | Y*AI | S | S | S | O | O | O | OH | OMe |
| 2377a-l | Y*AI | S | S | S | O | O | O | OMe | OMe |
| 2378a-l | Y*AI | S | S | S | O | O | O | H | OMe |
| 2379a-l | Y*AI | S | S | S | O | O | O | F | OMe |
| 2380a-l | Y*AI | S | S | S | O | O | O | OEt | OMe |
| 2381a-l | Y*AI | O | O | S | S | O | O | OH | OMe |
| 2382a-l | Y*AI | O | O | S | S | O | O | OMe | OMe |
| 2383a-l | Y*AI | O | O | S | S | O | O | H | OMe |
| 2384a-l | Y*AI | O | O | S | S | O | O | F | OMe |
| 2385a-l | Y*AI | O | O | S | S | O | O | OEt | OMe |
| 2386a-l | Y*AI | S | O | S | S | O | O | OH | OMe |
| 2387a-l | Y*AI | S | O | S | S | O | O | OMe | OMe |
| 2388a-l | Y*AI | S | O | S | S | O | O | H | OMe |
| 2389a-l | Y*AI | S | O | S | S | O | O | F | OMe |
| 2390a-l | Y*AI | S | O | S | S | O | O | OEt | OMe |
| 2391a-l | Y*AI | S | S | S | S | O | O | OH | OMe |
| 2392a-l | Y*AI | S | S | S | S | O | O | OMe | OMe |
| 2393a-l | Y*AI | S | S | S | S | O | O | H | OMe |
| 2394a-l | Y*AI | S | S | S | S | O | O | F | OMe |
| 2395a-l | Y*AI | S | S | S | S | O | O | OEt | OMe |
| 2396a-l | Y*AI | O | O | S | S | S | O | OH | OMe |
| 2397a-l | Y*AI | O | O | S | S | S | O | OMe | OMe |
| 2398a-l | Y*AI | O | O | S | S | S | O | H | OMe |
| 2399a-l | Y*AI | O | O | S | S | S | O | F | OMe |
| 2400a-l | Y*AI | O | O | S | S | S | O | OEt | OMe |
| 2401a-l | Y*AI | S | O | S | S | S | O | OH | OMe |
| 2402a-l | Y*AI | S | O | S | S | S | O | OMe | OMe |
| 2403a-l | Y*AI | S | O | S | S | S | O | H | OMe |
| 2404a-l | Y*AI | S | O | S | S | S | O | F | OMe |
| 2405a-l | Y*AI | S | O | S | S | S | O | OEt | OMe |
| 2406a-l | Y*AI | S | S | S | S | S | O | OH | OMe |
| 2407a-l | Y*AI | S | S | S | S | S | O | OMe | OMe |
| 2408a-l | Y*AI | S | S | S | S | S | O | H | OMe |
| 2409a-l | Y*AI | S | S | S | S | S | O | F | OMe |
| 2410a-l | Y*AI | S | S | S | S | S | O | OEt | OMe |
| 2411a-l | Y*AI | O | O | S | S | S | S | OH | OMe |
| 2412a-l | Y*AI | O | O | S | S | S | S | OMe | OMe |
| 2413a-l | Y*AI | O | O | S | S | S | S | H | OMe |
| 2414a-l | Y*AI | O | O | S | S | S | S | F | OMe |
| 2415a-l | Y*AI | O | O | S | S | S | S | OEt | OMe |
| 2416a-l | Y*AI | S | O | S | S | S | S | OH | OMe |
| 2417a-l | Y*AI | S | O | S | S | S | S | OMe | OMe |
| 2418a-l | Y*AI | S | O | S | S | S | S | H | OMe |
| 2419a-l | Y*AI | S | O | S | S | S | S | F | OMe |
| 2420a-l | Y*AI | S | O | S | S | S | S | OEt | OMe |
| 2421a-l | Y*AI | S | S | S | S | S | S | OH | OMe |
| 2422a-l | Y*AI | S | S | S | S | S | S | OMe | OMe |
| 2423a-l | Y*AI | S | S | S | S | S | S | H | OMe |
| 2424a-l | Y*AI | S | S | S | S | S | S | F | OMe |
| 2425a-l | Y*AI | S | S | S | S | S | S | OEt | OMe |
| 2426a-l | Y*AI | O | O | O | S | S | S | OH | OMe |
| 2427a-l | Y*AI | O | O | O | S | S | S | OMe | OMe |
| 2428a-l | Y*AI | O | O | O | S | S | S | H | OMe |
| 2429a-l | Y*AI | O | O | O | S | S | S | F | OMe |
| 2430a-l | Y*AI | O | O | O | S | S | S | OEt | OMe |
| 2431a-l | Y*AI | S | O | O | S | S | S | OH | OMe |
| 2432a-l | Y*AI | S | O | O | S | S | S | OMe | OMe |
| 2433a-l | Y*AI | S | O | O | S | S | S | H | OMe |
| 2434a-l | Y*AI | S | O | O | S | S | S | F | OMe |
| 2435a-l | Y*AI | S | O | O | S | S | S | OEt | OMe |
| 2436a-l | Y*AI | S | S | O | S | S | S | OH | OMe |
| 2437a-l | Y*AI | S | S | O | S | S | S | OMe | OMe |
| 2438a-l | Y*AI | S | S | O | S | S | S | H | OMe |
| 2439a-l | Y*AI | S | S | O | S | S | S | F | OMe |
| 2440a-l | Y*AI | S | S | O | S | S | S | OEt | OMe |
| 2441a-l | Y*AI | O | O | O | O | S | S | OH | OMe |
| 2442a-l | Y*AI | O | O | O | O | S | S | OMe | OMe |
| 2443a-l | Y*AI | O | O | O | O | S | S | H | OMe |
| 2444a-l | Y*AI | O | O | O | O | S | S | F | OMe |
| 2445a-l | Y*AI | O | O | O | O | S | S | OEt | OMe |
| 2446a-l | Y*AI | S | O | O | O | S | S | OH | OMe |
| 2447a-l | Y*AI | S | O | O | O | S | S | OMe | OMe |
| 2448a-l | Y*AI | S | O | O | O | S | S | H | OMe |
| 2449a-l | Y*AI | S | O | O | O | S | S | F | OMe |
| 2450a-l | Y*AI | S | O | O | O | S | S | OEt | OMe |
| 2451a-l | Y*AI | S | S | O | O | S | S | OH | OMe |
| 2452a-l | Y*AI | S | S | O | O | S | S | OMe | OMe |
| 2453a-l | Y*AI | S | S | O | O | S | S | H | OMe |
| 2454a-l | Y*AI | S | S | O | O | S | S | F | OMe |
| 2455a-l | Y*AI | S | S | O | O | S | S | OEt | OMe |
| 2456a-l | Y*AI | O | O | O | O | O | S | OH | OMe |
| 2457a-l | Y*AI | O | O | O | O | O | S | OMe | OMe |
| 2458a-l | Y*AI | O | O | O | O | O | S | H | OMe |
| 2459a-l | Y*AI | O | O | O | O | O | S | F | OMe |
| 2460a-l | Y*AI | O | O | O | O | O | S | OEt | OMe |
| 2461a-l | Y*AI | S | O | O | O | O | S | OH | OMe |
| 2462a-l | Y*AI | S | O | O | O | O | S | OMe | OMe |
| 2463a-l | Y*AI | S | O | O | O | O | S | H | OMe |
| 2464a-l | Y*AI | S | O | O | O | O | S | F | OMe |
| 2465a-l | Y*AI | S | O | O | O | O | S | OEt | OMe |
| 2466a-l | Y*AI | S | S | O | O | O | S | OH | OMe |
| 2467a-l | Y*AI | S | S | O | O | O | S | OMe | OMe |
| 2468a-l | Y*AI | S | S | O | O | O | S | H | OMe |
| 2469a-l | Y*AI | S | S | O | O | O | S | F | OMe |
| 2470a-l | Y*AI | S | S | O | O | O | S | OEt | OMe |
| 2471a-l | Y*AI | O | O | S | O | S | O | OH | OMe |
| 2472a-l | Y*AI | O | O | S | O | S | O | OMe | OMe |
| 2473a-l | Y*AI | O | O | S | O | S | O | H | OMe |
| 2474a-l | Y*AI | O | O | S | O | S | O | F | OMe |
| 2475a-l | Y*AI | O | O | S | O | S | O | OEt | OMe |
| 2476a-l | Y*AI | S | O | S | O | S | O | OH | OMe |
| 2477a-l | Y*AI | S | O | S | O | S | O | OMe | OMe |
| 2478a-l | Y*AI | S | O | S | O | S | O | H | OMe |
| 2479a-l | Y*AI | S | O | S | O | S | O | F | OMe |
| 2480a-l | Y*AI | S | O | S | O | S | O | OEt | OMe |
| 2481a-l | Y*AI | S | S | S | O | S | O | OH | OMe |
| 2482a-l | Y*AI | S | S | S | O | S | O | OMe | OMe |
| 2483a-l | Y*AI | S | S | S | O | S | O | H | OMe |
| 2484a-l | Y*AI | S | S | S | O | S | O | F | OMe |
| 2485a-l | Y*AI | S | S | S | O | S | O | OEt | OMe |
| 2486a-l | Y*AI | O | O | O | S | O | S | OH | OMe |
| 2487a-l | Y*AI | O | O | O | S | O | S | OMe | OMe |
| 2488a-l | Y*AI | O | O | O | S | O | S | H | OMe |
| 2489a-l | Y*AI | O | O | O | S | O | S | F | OMe |
| 2490a-l | Y*AI | O | O | O | S | O | S | OEt | OMe |
| 2491a-l | Y*AI | S | O | O | S | O | S | OH | OMe |
| 2492a-l | Y*AI | S | O | O | S | O | S | OMe | OMe |
| 2493a-l | Y*AI | S | O | O | S | O | S | H | OMe |
| 2494a-l | Y*AI | S | O | O | S | O | S | F | OMe |
| 2495a-l | Y*AI | S | O | O | S | O | S | OEt | OMe |
| 2496a-l | Y*AI | S | S | O | S | O | S | OH | OMe |
| 2497a-l | Y*AI | S | S | O | S | O | S | OMe | OMe |
| 2498a-l | Y*AI | S | S | O | S | O | S | H | OMe |
| 2499a-l | Y*AI | S | S | O | S | O | S | F | OMe |
| 2500a-l | Y*AI | S | S | O | S | O | S | OEt | OMe |
| 2501a-l | Y*AT | O | O | O | O | O | O | OH | OH |
| 2502a-l | Y*AT | O | O | O | O | O | O | OMe | OH |
| 2503a-l | Y*AT | O | O | O | O | O | O | H | OH |
| 2504a-l | Y*AT | O | O | O | O | O | O | F | OH |
| 2505a-l | Y*AT | O | O | O | O | O | O | OEt | OH |
| 2506a-l | Y*AT | S | O | O | O | O | O | OH | OH |
| 2507a-l | Y*AT | S | O | O | O | O | O | OMe | OH |
| 2508a-l | Y*AT | S | O | O | O | O | O | H | OH |
| 2509a-l | Y*AT | S | O | O | O | O | O | F | OH |
| 2510a-l | Y*AT | S | O | O | O | O | O | OEt | OH |
| 2511a-l | Y*AT | S | S | O | O | O | O | OH | OH |
| 2512a-l | Y*AT | S | S | O | O | O | O | OMe | OH |
| 2513a-l | Y*AT | S | S | O | O | O | O | H | OH |
| 2514a-l | Y*AT | S | S | O | O | O | O | F | OH |
| 2515a-l | Y*AT | S | S | O | O | O | O | OEt | OH |
| 2516a-l | Y*AT | O | O | S | O | O | O | OH | OH |
| 2517a-l | Y*AT | O | O | S | O | O | O | OMe | OH |
| 2518a-l | Y*AT | O | O | S | O | O | O | H | OH |
| 2519a-l | Y*AT | O | O | S | O | O | O | F | OH |
| 2520a-l | Y*AT | O | O | S | O | O | O | OEt | OH |
| 2521a-l | Y*AT | S | O | S | O | O | O | OH | OH |
| 2522a-l | Y*AT | S | O | S | O | O | O | OMe | OH |
| 2523a-l | Y*AT | S | O | S | O | O | O | H | OH |
| 2524a-l | Y*AT | S | O | S | O | O | O | F | OH |
| 2525a-l | Y*AT | S | O | S | O | O | O | OEt | OH |
| 2526a-l | Y*AT | S | S | S | O | O | O | OH | OH |
| 2527a-l | Y*AT | S | S | S | O | O | O | OMe | OH |
| 2528a-l | Y*AT | S | S | S | O | O | O | H | OH |
| 2529a-l | Y*AT | S | S | S | O | O | O | F | OH |
| 2530a-l | Y*AT | S | S | S | O | O | O | OEt | OH |
| 2531a-l | Y*AT | O | O | S | S | O | O | OH | OH |
| 2532a-l | Y*AT | O | O | S | S | O | O | OMe | OH |
| 2533a-l | Y*AT | O | O | S | S | O | O | H | OH |
| 2534a-l | Y*AT | O | O | S | S | O | O | F | OH |
| 2535a-l | Y*AT | O | O | S | S | O | O | OEt | OH |
| 2536a-l | Y*AT | S | O | S | S | O | O | OH | OH |
| 2537a-l | Y*AT | S | O | S | S | O | O | OMe | OH |
| 2538a-l | Y*AT | S | O | S | S | O | O | H | OH |
| 2539a-l | Y*AT | S | O | S | S | O | O | F | OH |
| 2540a-l | Y*AT | S | O | S | S | O | O | OEt | OH |
| 2541a-l | Y*AT | S | S | S | S | O | O | OH | OH |
| 2542a-l | Y*AT | S | S | S | S | O | O | OMe | OH |
| 2543a-l | Y*AT | S | S | S | S | O | O | H | OH |
| 2544a-l | Y*AT | S | S | S | S | O | O | F | OH |
| 2545a-l | Y*AT | S | S | S | S | O | O | OEt | OH |
| 2546a-l | Y*AT | O | O | S | S | S | O | OH | OH |
| 2547a-l | Y*AT | O | O | S | S | S | O | OMe | OH |
| 2548a-l | Y*AT | O | O | S | S | S | O | H | OH |
| 2549a-l | Y*AT | O | O | S | S | S | O | F | OH |
| 2550a-l | Y*AT | O | O | S | S | S | O | OEt | OH |
| 2551a-l | Y*AT | S | O | S | S | S | O | OH | OH |
| 2552a-l | Y*AT | S | O | S | S | S | O | OMe | OH |
| 2553a-l | Y*AT | S | O | S | S | S | O | H | OH |
| 2554a-l | Y*AT | S | O | S | S | S | O | F | OH |
| 2555a-l | Y*AT | S | O | S | S | S | O | OEt | OH |
| 2556a-l | Y*AT | S | S | S | S | S | O | OH | OH |
| 2557a-l | Y*AT | S | S | S | S | S | O | OMe | OH |
| 2558a-l | Y*AT | S | S | S | S | S | O | H | OH |
| 2559a-l | Y*AT | S | S | S | S | S | O | F | OH |
| 2560a-l | Y*AT | S | S | S | S | S | O | OEt | OH |
| 2561a-l | Y*AT | O | O | S | S | S | S | OH | OH |
| 2562a-l | Y*AT | O | O | S | S | S | S | OMe | OH |
| 2563a-l | Y*AT | O | O | S | S | S | S | H | OH |
| 2564a-l | Y*AT | O | O | S | S | S | S | F | OH |
| 2565a-l | Y*AT | O | O | S | S | S | S | OEt | OH |
| 2566a-l | Y*AT | S | O | S | S | S | S | OH | OH |
| 2567a-l | Y*AT | S | O | S | S | S | S | OMe | OH |
| 2568a-l | Y*AT | S | O | S | S | S | S | H | OH |
| 2569a-l | Y*AT | S | O | S | S | S | S | F | OH |
| 2570a-l | Y*AT | S | O | S | S | S | S | OEt | OH |
| 2571a-l | Y*AT | S | S | S | S | S | S | OH | OH |
| 2572a-l | Y*AT | S | S | S | S | S | S | OMe | OH |
| 2573a-l | Y*AT | S | S | S | S | S | S | H | OH |
| 2574a-l | Y*AT | S | S | S | S | S | S | F | OH |
| 2575a-l | Y*AT | S | S | S | S | S | S | OEt | OH |
| 2576a-l | Y*AT | O | O | O | S | S | S | OH | OH |
| 2577a-l | Y*AT | O | O | O | S | S | S | OMe | OH |
| 2578a-l | Y*AT | O | O | O | S | S | S | H | OH |
| 2579a-l | Y*AT | O | O | O | S | S | S | F | OH |
| 2580a-l | Y*AT | O | O | O | S | S | S | OEt | OH |
| 2581a-l | Y*AT | S | O | O | S | S | S | OH | OH |
| 2582a-l | Y*AT | S | O | O | S | S | S | OMe | OH |
| 2583a-l | Y*AT | S | O | O | S | S | S | H | OH |
| 2584a-l | Y*AT | S | O | O | S | S | S | F | OH |
| 2585a-l | Y*AT | S | O | O | S | S | S | OEt | OH |
| 2586a-l | Y*AT | S | S | O | S | S | S | OH | OH |
| 2587a-l | Y*AT | S | S | O | S | S | S | OMe | OH |
| 2588a-l | Y*AT | S | S | O | S | S | S | H | OH |
| 2589a-l | Y*AT | S | S | O | S | S | S | F | OH |
| 2590a-l | Y*AT | S | S | O | S | S | S | OEt | OH |
| 2591a-l | Y*AT | O | O | O | O | S | S | OH | OH |
| 2592a-l | Y*AT | O | O | O | O | S | S | OMe | OH |
| 2593a-l | Y*AT | O | O | O | O | S | S | H | OH |
| 2594a-l | Y*AT | O | O | O | O | S | S | F | OH |
| 2595a-l | Y*AT | O | O | O | O | S | S | OEt | OH |
| 2596a-l | Y*AT | S | O | O | O | S | S | OH | OH |
| 2597a-l | Y*AT | S | O | O | O | S | S | OMe | OH |
| 2598a-l | Y*AT | S | O | O | O | S | S | H | OH |
| 2599a-l | Y*AT | S | O | O | O | S | S | F | OH |
| 2600a-l | Y*AT | S | O | O | O | S | S | OEt | OH |
| 2601a-l | Y*AT | S | S | O | O | S | S | OH | OH |
| 2602a-l | Y*AT | S | S | O | O | S | S | OMe | OH |
| 2603a-l | Y*AT | S | S | O | O | S | S | H | OH |
| 2604a-l | Y*AT | S | S | O | O | S | S | F | OH |
| 2605a-l | Y*AT | S | S | O | O | S | S | OEt | OH |
| 2606a-l | Y*AT | O | O | O | O | O | S | OH | OH |
| 2607a-l | Y*AT | O | O | O | O | O | S | OMe | OH |
| 2608a-l | Y*AT | O | O | O | O | O | S | H | OH |
| 2609a-l | Y*AT | O | O | O | O | O | S | F | OH |
| 2610a-l | Y*AT | O | O | O | O | O | S | OEt | OH |
| 2611a-l | Y*AT | S | O | O | O | O | S | OH | OH |
| 2612a-l | Y*AT | S | O | O | O | O | S | OMe | OH |
| 2613a-l | Y*AT | S | O | O | O | O | S | H | OH |
| 2614a-l | Y*AT | S | O | O | O | O | S | F | OH |
| 2615a-l | Y*AT | S | O | O | O | O | S | OEt | OH |
| 2616a-l | Y*AT | S | S | O | O | O | S | OH | OH |
| 2617a-l | Y*AT | S | S | O | O | O | S | OMe | OH |
| 2618a-l | Y*AT | S | S | O | O | O | S | H | OH |
| 2619a-l | Y*AT | S | S | O | O | O | S | F | OH |
| 2620a-l | Y*AT | S | S | O | O | O | S | OEt | OH |
| 2621a-l | Y*AT | O | O | S | O | S | O | OH | OH |
| 2622a-l | Y*AT | O | O | S | O | S | O | OMe | OH |
| 2623a-l | Y*AT | O | O | S | O | S | O | H | OH |
| 2624a-l | Y*AT | O | O | S | O | S | O | F | OH |
| 2625a-l | Y*AT | O | O | S | O | S | O | OEt | OH |
| 2626a-l | Y*AT | S | O | S | O | S | O | OH | OH |
| 2627a-l | Y*AT | S | O | S | O | S | O | OMe | OH |
| 2628a-l | Y*AT | S | O | S | O | S | O | H | OH |
| 2629a-l | Y*AT | S | O | S | O | S | O | F | OH |
| 2630a-l | Y*AT | S | O | S | O | S | O | OEt | OH |
| 2631a-l | Y*AT | S | S | S | O | S | O | OH | OH |
| 2632a-l | Y*AT | S | S | S | O | S | O | OMe | OH |
| 2633a-l | Y*AT | S | S | S | O | S | O | H | OH |
| 2634a-l | Y*AT | S | S | S | O | S | O | F | OH |
| 2635a-l | Y*AT | S | S | S | O | S | O | OEt | OH |
| 2636a-l | Y*AT | O | O | O | S | O | S | OH | OH |
| 2637a-l | Y*AT | O | O | O | S | O | S | OMe | OH |
| 2638a-l | Y*AT | O | O | O | S | O | S | H | OH |
| 2639a-l | Y*AT | O | O | O | S | O | S | F | OH |
| 2640a-l | Y*AT | O | O | O | S | O | S | OEt | OH |
| 2641a-l | Y*AT | S | O | O | S | O | S | OH | OH |
| 2642a-l | Y*AT | S | O | O | S | O | S | OMe | OH |
| 2643a-l | Y*AT | S | O | O | S | O | S | H | OH |
| 2644a-l | Y*AT | S | O | O | S | O | S | F | OH |
| 2645a-l | Y*AT | S | O | O | S | O | S | OEt | OH |
| 2646a-l | Y*AT | S | S | O | S | O | S | OH | OH |
| 2647a-l | Y*AT | S | S | O | S | O | S | OMe | OH |
| 2648a-l | Y*AT | S | S | O | S | O | S | H | OH |
| 2649a-l | Y*AT | S | S | O | S | O | S | F | OH |
| 2650a-l | Y*AT | S | S | O | S | O | S | OEt | OH |
| 2651a-l | Y*AT | O | O | O | O | O | O | OH | OMe |
| 2652a-l | Y*AT | O | O | O | O | O | O | OMe | OMe |
| 2653a-l | Y*AT | O | O | O | O | O | O | H | OMe |
| 2654a-l | Y*AT | O | O | O | O | O | O | F | OMe |
| 2655a-l | Y*AT | O | O | O | O | O | O | OEt | OMe |
| 2656a-l | Y*AT | S | O | O | O | O | O | OH | OMe |
| 2657a-l | Y*AT | S | O | O | O | O | O | OMe | OMe |
| 2658a-l | Y*AT | S | O | O | O | O | O | H | OMe |
| 2659a-l | Y*AT | S | O | O | O | O | O | F | OMe |
| 2660a-l | Y*AT | S | O | O | O | O | O | OEt | OMe |
| 2661a-l | Y*AT | S | S | O | O | O | O | OH | OMe |
| 2662a-l | Y*AT | S | S | O | O | O | O | OMe | OMe |
| 2663a-l | Y*AT | S | S | O | O | O | O | H | OMe |
| 2664a-l | Y*AT | S | S | O | O | O | O | F | OMe |
| 2665a-l | Y*AT | S | S | O | O | O | O | OEt | OMe |
| 2666a-l | Y*AT | O | O | S | O | O | O | OH | OMe |
| 2667a-l | Y*AT | O | O | S | O | O | O | OMe | OMe |
| 2668a-l | Y*AT | O | O | S | O | O | O | H | OMe |
| 2669a-l | Y*AT | O | O | S | O | O | O | F | OMe |
| 2670a-l | Y*AT | O | O | S | O | O | O | OEt | OMe |
| 2671a-l | Y*AT | S | O | S | O | O | O | OH | OMe |
| 2672a-l | Y*AT | S | O | S | O | O | O | OMe | OMe |
| 2673a-l | Y*AT | S | O | S | O | O | O | H | OMe |
| 2674a-l | Y*AT | S | O | S | O | O | O | F | OMe |
| 2675a-l | Y*AT | S | O | S | O | O | O | OEt | OMe |
| 2676a-l | Y*AT | S | S | S | O | O | O | OH | OMe |
| 2677a-l | Y*AT | S | S | S | O | O | O | OMe | OMe |
| 2678a-l | Y*AT | S | S | S | O | O | O | H | OMe |
| 2679a-l | Y*AT | S | S | S | O | O | O | F | OMe |
| 2680a-l | Y*AT | S | S | S | O | O | O | OEt | OMe |
| 2681a-l | Y*AT | O | O | S | S | O | O | OH | OMe |
| 2682a-l | Y*AT | O | O | S | S | O | O | OMe | OMe |
| 2683a-l | Y*AT | O | O | S | S | O | O | H | OMe |
| 2684a-l | Y*AT | O | O | S | S | O | O | F | OMe |
| 2685a-l | Y*AT | O | O | S | S | O | O | OEt | OMe |
| 2686a-l | Y*AT | S | O | S | S | O | O | OH | OMe |
| 2687a-l | Y*AT | S | O | S | S | O | O | OMe | OMe |
| 2688a-l | Y*AT | S | O | S | S | O | O | H | OMe |
| 2689a-l | Y*AT | S | O | S | S | O | O | F | OMe |
| 2690a-l | Y*AT | S | O | S | S | O | O | OEt | OMe |
| 2691a-l | Y*AT | S | S | S | S | O | O | OH | OMe |
| 2692a-l | Y*AT | S | S | S | S | O | O | OMe | OMe |
| 2693a-l | Y*AT | S | S | S | S | O | O | H | OMe |
| 2694a-l | Y*AT | S | S | S | S | O | O | F | OMe |
| 2695a-l | Y*AT | S | S | S | S | O | O | OEt | OMe |
| 2696a-l | Y*AT | O | O | S | S | S | O | OH | OMe |
| 2697a-l | Y*AT | O | O | S | S | S | O | OMe | OMe |
| 2698a-l | Y*AT | O | O | S | S | S | O | H | OMe |
| 2699a-l | Y*AT | O | O | S | S | S | O | F | OMe |
| 2700a-l | Y*AT | O | O | S | S | S | O | OEt | OMe |
| 2701a-l | Y*AT | S | O | S | S | S | O | OH | OMe |
| 2702a-l | Y*AT | S | O | S | S | S | O | OMe | OMe |
| 2703a-l | Y*AT | S | O | S | S | S | O | H | OMe |
| 2704a-l | Y*AT | S | O | S | S | S | O | F | OMe |
| 2705a-l | Y*AT | S | O | S | S | S | O | OEt | OMe |
| 2706a-l | Y*AT | S | S | S | S | S | O | OH | OMe |
| 2707a-l | Y*AT | S | S | S | S | S | O | OMe | OMe |
| 2708a-l | Y*AT | S | S | S | S | S | O | H | OMe |
| 2709a-l | Y*AT | S | S | S | S | S | O | F | OMe |
| 2710a-l | Y*AT | S | S | S | S | S | O | OEt | OMe |
| 2711a-l | Y*AT | O | O | S | S | S | S | OH | OMe |
| 2712a-l | Y*AT | O | O | S | S | S | S | OMe | OMe |
| 2713a-l | Y*AT | O | O | S | S | S | S | H | OMe |
| 2714a-l | Y*AT | O | O | S | S | S | S | F | OMe |
| 2715a-l | Y*AT | O | O | S | S | S | S | OEt | OMe |
| 2716a-l | Y*AT | S | O | S | S | S | S | OH | OMe |
| 2717a-l | Y*AT | S | O | S | S | S | S | OMe | OMe |
| 2718a-l | Y*AT | S | O | S | S | S | S | H | OMe |
| 2719a-l | Y*AT | S | O | S | S | S | S | F | OMe |
| 2720a-l | Y*AT | S | O | S | S | S | S | OEt | OMe |
| 2721a-l | Y*AT | S | S | S | S | S | S | OH | OMe |
| 2722a-l | Y*AT | S | S | S | S | S | S | OMe | OMe |
| 2723a-l | Y*AT | S | S | S | S | S | S | H | OMe |
| 2724a-l | Y*AT | S | S | S | S | S | S | F | OMe |
| 2725a-l | Y*AT | S | S | S | S | S | S | OEt | OMe |
| 2726a-l | Y*AT | O | O | O | S | S | S | OH | OMe |
| 2727a-l | Y*AT | O | O | O | S | S | S | OMe | OMe |
| 2728a-l | Y*AT | O | O | O | S | S | S | H | OMe |
| 2729a-l | Y*AT | O | O | O | S | S | S | F | OMe |
| 2730a-l | Y*AT | O | O | O | S | S | S | OEt | OMe |
| 2731a-l | Y*AT | S | O | O | S | S | S | OH | OMe |
| 2732a-l | Y*AT | S | O | O | S | S | S | OMe | OMe |
| 2733a-l | Y*AT | S | O | O | S | S | S | H | OMe |
| 2734a-l | Y*AT | S | O | O | S | S | S | F | OMe |
| 2735a-l | Y*AT | S | O | O | S | S | S | OEt | OMe |
| 2736a-l | Y*AT | S | S | O | S | S | S | OH | OMe |
| 2737a-l | Y*AT | S | S | O | S | S | S | OMe | OMe |
| 2738a-l | Y*AT | S | S | O | S | S | S | H | OMe |
| 2739a-l | Y*AT | S | S | O | S | S | S | F | OMe |
| 2740a-l | Y*AT | S | S | O | S | S | S | OEt | OMe |
| 2741a-l | Y*AT | O | O | O | O | S | S | OH | OMe |
| 2742a-l | Y*AT | O | O | O | O | S | S | OMe | OMe |
| 2743a-l | Y*AT | O | O | O | O | S | S | H | OMe |
| 2744a-l | Y*AT | O | O | O | O | S | S | F | OMe |
| 2745a-l | Y*AT | O | O | O | O | S | S | OEt | OMe |
| 2746a-l | Y*AT | S | O | O | O | S | S | OH | OMe |
| 2747a-l | Y*AT | S | O | O | O | S | S | OMe | OMe |
| 2748a-l | Y*AT | S | O | O | O | S | S | H | OMe |
| 2749a-l | Y*AT | S | O | O | O | S | S | F | OMe |
| 2750a-l | Y*AT | S | O | O | O | S | S | OEt | OMe |
| 2751a-l | Y*AT | S | S | O | O | S | S | OH | OMe |
| 2752a-l | Y*AT | S | S | O | O | S | S | OMe | OMe |
| 2753a-l | Y*AT | S | S | O | O | S | S | H | OMe |
| 2754a-l | Y*AT | S | S | O | O | S | S | F | OMe |
| 2755a-l | Y*AT | S | S | O | O | S | S | OEt | OMe |
| 2756a-l | Y*AT | O | O | O | O | O | S | OH | OMe |
| 2757a-l | Y*AT | O | O | O | O | O | S | OMe | OMe |
| 2758a-l | Y*AT | O | O | O | O | O | S | H | OMe |
| 2759a-l | Y*AT | O | O | O | O | O | S | F | OMe |
| 2760a-l | Y*AT | O | O | O | O | O | S | OEt | OMe |
| 2761a-l | Y*AT | S | O | O | O | O | S | OH | OMe |
| 2762a-l | Y*AT | S | O | O | O | O | S | OMe | OMe |
| 2763a-l | Y*AT | S | O | O | O | O | S | H | OMe |
| 2764a-l | Y*AT | S | O | O | O | O | S | F | OMe |
| 2765a-l | Y*AT | S | O | O | O | O | S | OEt | OMe |
| 2766a-l | Y*AT | S | S | O | O | O | S | OH | OMe |
| 2767a-l | Y*AT | S | S | O | O | O | S | OMe | OMe |
| 2768a-l | Y*AT | S | S | O | O | O | S | H | OMe |
| 2769a-l | Y*AT | S | S | O | O | O | S | F | OMe |
| 2770a-l | Y*AT | S | S | O | O | O | S | OEt | OMe |
| 2771a-l | Y*AT | O | O | S | O | S | O | OH | OMe |
| 2772a-l | Y*AT | O | O | S | O | S | O | OMe | OMe |
| 2773a-l | Y*AT | O | O | S | O | S | O | H | OMe |
| 2774a-l | Y*AT | O | O | S | O | S | O | F | OMe |
| 2775a-l | Y*AT | O | O | S | O | S | O | OEt | OMe |
| 2776a-l | Y*AT | S | O | S | O | S | O | OH | OMe |
| 2777a-l | Y*AT | S | O | S | O | S | O | OMe | OMe |
| 2778a-l | Y*AT | S | O | S | O | S | O | H | OMe |
| 2779a-l | Y*AT | S | O | S | O | S | O | F | OMe |
| 2780a-l | Y*AT | S | O | S | O | S | O | OEt | OMe |
| 2781a-l | Y*AT | S | S | S | O | S | O | OH | OMe |
| 2782a-l | Y*AT | S | S | S | O | S | O | OMe | OMe |
| 2783a-l | Y*AT | S | S | S | O | S | O | H | OMe |
| 2784a-l | Y*AT | S | S | S | O | S | O | F | OMe |
| 2785a-l | Y*AT | S | S | S | O | S | O | OEt | OMe |
| 2786a-l | Y*AT | O | O | O | S | O | S | OH | OMe |
| 2787a-l | Y*AT | O | O | O | S | O | S | OMe | OMe |
| 2788a-l | Y*AT | O | O | O | S | O | S | H | OMe |
| 2789a-l | Y*AT | O | O | O | S | O | S | F | OMe |
| 2790a-l | Y*AT | O | O | O | S | O | S | OEt | OMe |
| 2791a-l | Y*AT | S | O | O | S | O | S | OH | OMe |
| 2792a-l | Y*AT | S | O | O | S | O | S | OMe | OMe |
| 2793a-l | Y*AT | S | O | O | S | O | S | H | OMe |
| 2794a-l | Y*AT | S | O | O | S | O | S | F | OMe |
| 2795a-l | Y*AT | S | O | O | S | O | S | OEt | OMe |
| 2796a-l | Y*AT | S | S | O | S | O | S | OH | OMe |
| 2797a-l | Y*AT | S | S | O | S | O | S | OMe | OMe |
| 2798a-l | Y*AT | S | S | O | S | O | S | H | OMe |
| 2799a-l | Y*AT | S | S | O | S | O | S | F | OMe |
| 2800a-l | Y*AT | S | S | O | S | O | S | OEt | OMe |
| 2801a-l | Y*AE | O | O | O | O | O | O | OH | OH |
| 2802a-l | Y*AE | O | O | O | O | O | O | OMe | OH |
| 2803a-l | Y*AE | O | O | O | O | O | O | H | OH |
| 2804a-l | Y*AE | O | O | O | O | O | O | F | OH |
| 2805a-l | Y*AE | O | O | O | O | O | O | OEt | OH |
| 2806a-l | Y*AE | S | O | O | O | O | O | OH | OH |
| 2807a-l | Y*AE | S | O | O | O | O | O | OMe | OH |
| 2808a-l | Y*AE | S | O | O | O | O | O | H | OH |
| 2809a-l | Y*AE | S | O | O | O | O | O | F | OH |
| 2810a-l | Y*AE | S | O | O | O | O | O | OEt | OH |
| 2811a-l | Y*AE | S | S | O | O | O | O | OH | OH |
| 2812a-l | Y*AE | S | S | O | O | O | O | OMe | OH |
| 2813a-l | Y*AE | S | S | O | O | O | O | H | OH |
| 2814a-l | Y*AE | S | S | O | O | O | O | F | OH |
| 2815a-l | Y*AE | S | S | O | O | O | O | OEt | OH |
| 2816a-l | Y*AE | O | O | S | O | O | O | OH | OH |
| 2817a-l | Y*AE | O | O | S | O | O | O | OMe | OH |
| 2818a-l | Y*AE | O | O | S | O | O | O | H | OH |
| 2819a-l | Y*AE | O | O | S | O | O | O | F | OH |
| 2820a-l | Y*AE | O | O | S | O | O | O | OEt | OH |
| 2821a-l | Y*AE | S | O | S | O | O | O | OH | OH |
| 2822a-l | Y*AE | S | O | S | O | O | O | OMe | OH |
| 2823a-l | Y*AE | S | O | S | O | O | O | H | OH |
| 2824a-l | Y*AE | S | O | S | O | O | O | F | OH |
| 2825a-l | Y*AE | S | O | S | O | O | O | OEt | OH |
| 2826a-l | Y*AE | S | S | S | O | O | O | OH | OH |
| 2827a-l | Y*AE | S | S | S | O | O | O | OMe | OH |
| 2828a-l | Y*AE | S | S | S | O | O | O | H | OH |
| 2829a-l | Y*AE | S | S | S | O | O | O | F | OH |
| 2830a-l | Y*AE | S | S | S | O | O | O | OEt | OH |
| 2831a-l | Y*AE | O | O | S | S | O | O | OH | OH |
| 2832a-l | Y*AE | O | O | S | S | O | O | OMe | OH |
| 2833a-l | Y*AE | O | O | S | S | O | O | H | OH |
| 2834a-l | Y*AE | O | O | S | S | O | O | F | OH |
| 2835a-l | Y*AE | O | O | S | S | O | O | OEt | OH |
| 2836a-l | Y*AE | S | O | S | S | O | O | OH | OH |
| 2837a-l | Y*AE | S | O | S | S | O | O | OMe | OH |
| 2838a-l | Y*AE | S | O | S | S | O | O | H | OH |
| 2839a-l | Y*AE | S | O | S | S | O | O | F | OH |
| 2840a-l | Y*AE | S | O | S | S | O | O | OEt | OH |
| 2841a-l | Y*AE | S | S | S | S | O | O | OH | OH |
| 2842a-l | Y*AE | S | S | S | S | O | O | OMe | OH |
| 2843a-l | Y*AE | S | S | S | S | O | O | H | OH |
| 2844a-l | Y*AE | S | S | S | S | O | O | F | OH |
| 2845a-l | Y*AE | S | S | S | S | O | O | OEt | OH |
| 2846a-l | Y*AE | O | O | S | S | S | O | OH | OH |
| 2847a-l | Y*AE | O | O | S | S | S | O | OMe | OH |
| 2848a-l | Y*AE | O | O | S | S | S | O | H | OH |
| 2849a-l | Y*AE | O | O | S | S | S | O | F | OH |
| 2850a-l | Y*AE | O | O | S | S | S | O | OEt | OH |
| 2851a-l | Y*AE | S | O | S | S | S | O | OH | OH |
| 2852a-l | Y*AE | S | O | S | S | S | O | OMe | OH |
| 2853a-l | Y*AE | S | O | S | S | S | O | H | OH |
| 2854a-l | Y*AE | S | O | S | S | S | O | F | OH |
| 2855a-l | Y*AE | S | O | S | S | S | O | OEt | OH |
| 2856a-l | Y*AE | S | S | S | S | S | O | OH | OH |
| 2857a-l | Y*AE | S | S | S | S | S | O | OMe | OH |
| 2858a-l | Y*AE | S | S | S | S | S | O | H | OH |
| 2859a-l | Y*AE | S | S | S | S | S | O | F | OH |
| 2860a-l | Y*AE | S | S | S | S | S | O | OEt | OH |
| 2861a-l | Y*AE | O | O | S | S | S | S | OH | OH |
| 2862a-l | Y*AE | O | O | S | S | S | S | OMe | OH |
| 2863a-l | Y*AE | O | O | S | S | S | S | H | OH |
| 2864a-l | Y*AE | O | O | S | S | S | S | F | OH |
| 2865a-l | Y*AE | O | O | S | S | S | S | OEt | OH |
| 2866a-l | Y*AE | S | O | S | S | S | S | OH | OH |
| 2867a-l | Y*AE | S | O | S | S | S | S | OMe | OH |
| 2868a-l | Y*AE | S | O | S | S | S | S | H | OH |
| 2869a-l | Y*AE | S | O | S | S | S | S | F | OH |
| 2870a-l | Y*AE | S | O | S | S | S | S | OEt | OH |
| 2871a-l | Y*AE | S | S | S | S | S | S | OH | OH |
| 2872a-l | Y*AE | S | S | S | S | S | S | OMe | OH |
| 2873a-l | Y*AE | S | S | S | S | S | S | H | OH |
| 2874a-l | Y*AE | S | S | S | S | S | S | F | OH |
| 2875a-l | Y*AE | S | S | S | S | S | S | OEt | OH |
| 2876a-l | Y*AE | O | O | O | S | S | S | OH | OH |
| 2877a-l | Y*AE | O | O | O | S | S | S | OMe | OH |
| 2878a-l | Y*AE | O | O | O | S | S | S | H | OH |
| 2879a-l | Y*AE | O | O | O | S | S | S | F | OH |
| 2880a-l | Y*AE | O | O | O | S | S | S | OEt | OH |
| 2881a-l | Y*AE | S | O | O | S | S | S | OH | OH |
| 2882a-l | Y*AE | S | O | O | S | S | S | OMe | OH |
| 2883a-l | Y*AE | S | O | O | S | S | S | H | OH |
| 2884a-l | Y*AE | S | O | O | S | S | S | F | OH |
| 2885a-l | Y*AE | S | O | O | S | S | S | OEt | OH |
| 2886a-l | Y*AE | S | S | O | S | S | S | OH | OH |
| 2887a-l | Y*AE | S | S | O | S | S | S | OMe | OH |
| 2888a-l | Y*AE | S | S | O | S | S | S | H | OH |
| 2889a-l | Y*AE | S | S | O | S | S | S | F | OH |
| 2890a-l | Y*AE | S | S | O | S | S | S | OEt | OH |
| 2891a-l | Y*AE | O | O | O | O | S | S | OH | OH |
| 2892a-l | Y*AE | O | O | O | O | S | S | OMe | OH |
| 2893a-l | Y*AE | O | O | O | O | S | S | H | OH |
| 2894a-l | Y*AE | O | O | O | O | S | S | F | OH |
| 2895a-l | Y*AE | O | O | O | O | S | S | OEt | OH |
| 2896a-l | Y*AE | S | O | O | O | S | S | OH | OH |
| 2897a-l | Y*AE | S | O | O | O | S | S | OMe | OH |
| 2898a-l | Y*AE | S | O | O | O | S | S | H | OH |
| 2899a-l | Y*AE | S | O | O | O | S | S | F | OH |
| 2900a-l | Y*AE | S | O | O | O | S | S | OEt | OH |
| 2901a-l | Y*AE | S | S | O | O | S | S | OH | OH |
| 2902a-l | Y*AE | S | S | O | O | S | S | OMe | OH |
| 2903a-l | Y*AE | S | S | O | O | S | S | H | OH |
| 2904a-l | Y*AE | S | S | O | O | S | S | F | OH |
| 2905a-l | Y*AE | S | S | O | O | S | S | OEt | OH |
| 2906a-l | Y*AE | O | O | O | O | O | S | OH | OH |
| 2907a-l | Y*AE | O | O | O | O | O | S | OMe | OH |
| 2908a-l | Y*AE | O | O | O | O | O | S | H | OH |
| 2909a-l | Y*AE | O | O | O | O | O | S | F | OH |
| 2910a-l | Y*AE | O | O | O | O | O | S | OEt | OH |
| 2911a-l | Y*AE | S | O | O | O | O | S | OH | OH |
| 2912a-l | Y*AE | S | O | O | O | O | S | OMe | OH |
| 2913a-l | Y*AE | S | O | O | O | O | S | H | OH |
| 2914a-l | Y*AE | S | O | O | O | O | S | F | OH |
| 2915a-l | Y*AE | S | O | O | O | O | S | OEt | OH |
| 2916a-l | Y*AE | S | S | O | O | O | S | OH | OH |
| 2917a-l | Y*AE | S | S | O | O | O | S | OMe | OH |
| 2918a-l | Y*AE | S | S | O | O | O | S | H | OH |
| 2919a-l | Y*AE | S | S | O | O | O | S | F | OH |
| 2920a-l | Y*AE | S | S | O | O | O | S | OEt | OH |
| 2921a-l | Y*AE | O | O | S | O | S | O | OH | OH |
| 2922a-l | Y*AE | O | O | S | O | S | O | OMe | OH |
| 2923a-l | Y*AE | O | O | S | O | S | O | H | OH |
| 2924a-l | Y*AE | O | O | S | O | S | O | F | OH |
| 2925a-l | Y*AE | O | O | S | O | S | O | OEt | OH |
| 2926a-l | Y*AE | S | O | S | O | S | O | OH | OH |
| 2927a-l | Y*AE | S | O | S | O | S | O | OMe | OH |
| 2928a-l | Y*AE | S | O | S | O | S | O | H | OH |
| 2929a-l | Y*AE | S | O | S | O | S | O | F | OH |
| 2930a-l | Y*AE | S | O | S | O | S | O | OEt | OH |
| 2931a-l | Y*AE | S | S | S | O | S | O | OH | OH |
| 2932a-l | Y*AE | S | S | S | O | S | O | OMe | OH |
| 2933a-l | Y*AE | S | S | S | O | S | O | H | OH |
| 2934a-l | Y*AE | S | S | S | O | S | O | F | OH |
| 2935a-l | Y*AE | S | S | S | O | S | O | OEt | OH |
| 2936a-l | Y*AE | O | O | O | S | O | S | OH | OH |
| 2937a-l | Y*AE | O | O | O | S | O | S | OMe | OH |
| 2938a-l | Y*AE | O | O | O | S | O | S | H | OH |
| 2939a-l | Y*AE | O | O | O | S | O | S | F | OH |
| 2940a-l | Y*AE | O | O | O | S | O | S | OEt | OH |
| 2941a-l | Y*AE | S | O | O | S | O | S | OH | OH |
| 2942a-l | Y*AE | S | O | O | S | O | S | OMe | OH |
| 2943a-l | Y*AE | S | O | O | S | O | S | H | OH |
| 2944a-l | Y*AE | S | O | O | S | O | S | F | OH |
| 2945a-l | Y*AE | S | O | O | S | O | S | OEt | OH |
| 2946a-l | Y*AE | S | S | O | S | O | S | OH | OH |
| 2947a-l | Y*AE | S | S | O | S | O | S | OMe | OH |
| 2948a-l | Y*AE | S | S | O | S | O | S | H | OH |
| 2949a-l | Y*AE | S | S | O | S | O | S | F | OH |
| 2950a-l | Y*AE | S | S | O | S | O | S | OEt | OH |
| 2951a-l | Y*AE | O | O | O | O | O | O | OH | OMe |
| 2952a-l | Y*AE | O | O | O | O | O | O | OMe | OMe |
| 2953a-l | Y*AE | O | O | O | O | O | O | H | OMe |
| 2954a-l | Y*AE | O | O | O | O | O | O | F | OMe |
| 2955a-l | Y*AE | O | O | O | O | O | O | OEt | OMe |
| 2956a-l | Y*AE | S | O | O | O | O | O | OH | OMe |
| 2957a-l | Y*AE | S | O | O | O | O | O | OMe | OMe |
| 2958a-l | Y*AE | S | O | O | O | O | O | H | OMe |
| 2959a-l | Y*AE | S | O | O | O | O | O | F | OMe |
| 2960a-l | Y*AE | S | O | O | O | O | O | OEt | OMe |
| 2961a-l | Y*AE | S | S | O | O | O | O | OH | OMe |
| 2962a-l | Y*AE | S | S | O | O | O | O | OMe | OMe |
| 2963a-l | Y*AE | S | S | O | O | O | O | H | OMe |
| 2964a-l | Y*AE | S | S | O | O | O | O | F | OMe |
| 2965a-l | Y*AE | S | S | O | O | O | O | OEt | OMe |
| 2966a-l | Y*AE | O | O | S | O | O | O | OH | OMe |
| 2967a-l | Y*AE | O | O | S | O | O | O | OMe | OMe |
| 2968a-l | Y*AE | O | O | S | O | O | O | H | OMe |
| 2969a-l | Y*AE | O | O | S | O | O | O | F | OMe |
| 2970a-l | Y*AE | O | O | S | O | O | O | OEt | OMe |
| 2971a-l | Y*AE | S | O | S | O | O | O | OH | OMe |
| 2972a-l | Y*AE | S | O | S | O | O | O | OMe | OMe |
| 2973a-l | Y*AE | S | O | S | O | O | O | H | OMe |
| 2974a-l | Y*AE | S | O | S | O | O | O | F | OMe |
| 2975a-l | Y*AE | S | O | S | O | O | O | OEt | OMe |
| 2976a-l | Y*AE | S | S | S | O | O | O | OH | OMe |
| 2977a-l | Y*AE | S | S | S | O | O | O | OMe | OMe |
| 2978a-l | Y*AE | S | S | S | O | O | O | H | OMe |
| 2979a-l | Y*AE | S | S | S | O | O | O | F | OMe |
| 2980a-l | Y*AE | S | S | S | O | O | O | OEt | OMe |
| 2981a-l | Y*AE | O | O | S | S | O | O | OH | OMe |
| 2982a-l | Y*AE | O | O | S | S | O | O | OMe | OMe |
| 2983a-l | Y*AE | O | O | S | S | O | O | H | OMe |
| 2984a-l | Y*AE | O | O | S | S | O | O | F | OMe |
| 2985a-l | Y*AE | O | O | S | S | O | O | OEt | OMe |
| 2986a-l | Y*AE | S | O | S | S | O | O | OH | OMe |
| 2987a-l | Y*AE | S | O | S | S | O | O | OMe | OMe |
| 2988a-l | Y*AE | S | O | S | S | O | O | H | OMe |
| 2989a-l | Y*AE | S | O | S | S | O | O | F | OMe |
| 2990a-l | Y*AE | S | O | S | S | O | O | OEt | OMe |
| 2991a-l | Y*AE | S | S | S | S | O | O | OH | OMe |
| 2992a-l | Y*AE | S | S | S | S | O | O | OMe | OMe |
| 2993a-l | Y*AE | S | S | S | S | O | O | H | OMe |
| 2994a-l | Y*AE | S | S | S | S | O | O | F | OMe |
| 2995a-l | Y*AE | S | S | S | S | O | O | OEt | OMe |
| 2996a-l | Y*AE | O | O | S | S | S | O | OH | OMe |
| 2997a-l | Y*AE | O | O | S | S | S | O | OMe | OMe |
| 2998a-l | Y*AE | O | O | S | S | S | O | H | OMe |
| 2999a-l | Y*AE | O | O | S | S | S | O | F | OMe |
| 3000a-l | Y*AE | O | O | S | S | S | O | OEt | OMe |
| 3001a-l | Y*AE | S | O | S | S | S | O | OH | OMe |
| 3002a-l | Y*AE | S | O | S | S | S | O | OMe | OMe |
| 3003a-l | Y*AE | S | O | S | S | S | O | H | OMe |
| 3004a-l | Y*AE | S | O | S | S | S | O | F | OMe |
| 3005a-l | Y*AE | S | O | S | S | S | O | OEt | OMe |
| 3006a-l | Y*AE | S | S | S | S | S | O | OH | OMe |
| 3007a-l | Y*AE | S | S | S | S | S | O | OMe | OMe |
| 3008a-l | Y*AE | S | S | S | S | S | O | H | OMe |
| 3009a-l | Y*AE | S | S | S | S | S | O | F | OMe |
| 3010a-l | Y*AE | S | S | S | S | S | O | OEt | OMe |
| 3011a-l | Y*AE | O | O | S | S | S | S | OH | OMe |
| 3012a-l | Y*AE | O | O | S | S | S | S | OMe | OMe |
| 3013a-l | Y*AE | O | O | S | S | S | S | H | OMe |
| 3014a-l | Y*AE | O | O | S | S | S | S | F | OMe |
| 3015a-l | Y*AE | O | O | S | S | S | S | OEt | OMe |
| 3016a-l | Y*AE | S | O | S | S | S | S | OH | OMe |
| 3017a-l | Y*AE | S | O | S | S | S | S | OMe | OMe |
| 3018a-l | Y*AE | S | O | S | S | S | S | H | OMe |
| 3019a-l | Y*AE | S | O | S | S | S | S | F | OMe |
| 3020a-l | Y*AE | S | O | S | S | S | S | OEt | OMe |
| 3021a-l | Y*AE | S | S | S | S | S | S | OH | OMe |
| 3022a-l | Y*AE | S | S | S | S | S | S | OMe | OMe |
| 3023a-l | Y*AE | S | S | S | S | S | S | H | OMe |
| 3024a-l | Y*AE | S | S | S | S | S | S | F | OMe |
| 3025a-l | Y*AE | S | S | S | S | S | S | OEt | OMe |
| 3026a-l | Y*AE | O | O | O | S | S | S | OH | OMe |
| 3027a-l | Y*AE | O | O | O | S | S | S | OMe | OMe |
| 3028a-l | Y*AE | O | O | O | S | S | S | H | OMe |
| 3029a-l | Y*AE | O | O | O | S | S | S | F | OMe |
| 3030a-l | Y*AE | O | O | O | S | S | S | OEt | OMe |
| 3031a-l | Y*AE | S | O | O | S | S | S | OH | OMe |
| 3032a-l | Y*AE | S | O | O | S | S | S | OMe | OMe |
| 3033a-l | Y*AE | S | O | O | S | S | S | H | OMe |
| 3034a-l | Y*AE | S | O | O | S | S | S | F | OMe |
| 3035a-l | Y*AE | S | O | O | S | S | S | OEt | OMe |
| 3036a-l | Y*AE | S | S | O | S | S | S | OH | OMe |
| 3037a-l | Y*AE | S | S | O | S | S | S | OMe | OMe |
| 3038a-l | Y*AE | S | S | O | S | S | S | H | OMe |
| 3039a-l | Y*AE | S | S | O | S | S | S | F | OMe |
| 3040a-l | Y*AE | S | S | O | S | S | S | OEt | OMe |
| 3041a-l | Y*AE | O | O | O | O | S | S | OH | OMe |
| 3042a-l | Y*AE | O | O | O | O | S | S | OMe | OMe |
| 3043a-l | Y*AE | O | O | O | O | S | S | H | OMe |
| 3044a-l | Y*AE | O | O | O | O | S | S | F | OMe |
| 3045a-l | Y*AE | O | O | O | O | S | S | OEt | OMe |
| 3046a-l | Y*AE | S | O | O | O | S | S | OH | OMe |
| 3047a-l | Y*AE | S | O | O | O | S | S | OMe | OMe |
| 3048a-l | Y*AE | S | O | O | O | S | S | H | OMe |
| 3049a-l | Y*AE | S | O | O | O | S | S | F | OMe |
| 3050a-l | Y*AE | S | O | O | O | S | S | OEt | OMe |
| 3051a-l | Y*AE | S | S | O | O | S | S | OH | OMe |
| 3052a-l | Y*AE | S | S | O | O | S | S | OMe | OMe |
| 3053a-l | Y*AE | S | S | O | O | S | S | H | OMe |
| 3054a-l | Y*AE | S | S | O | O | S | S | F | OMe |
| 3055a-l | Y*AE | S | S | O | O | S | S | OEt | OMe |
| 3056a-l | Y*AE | O | O | O | O | O | S | OH | OMe |
| 3057a-l | Y*AE | O | O | O | O | O | S | OMe | OMe |
| 3058a-l | Y*AE | O | O | O | O | O | S | H | OMe |
| 3059a-l | Y*AE | O | O | O | O | O | S | F | OMe |
| 3060a-l | Y*AE | O | O | O | O | O | S | OEt | OMe |
| 3061a-l | Y*AE | S | O | O | O | O | S | OH | OMe |
| 3062a-l | Y*AE | S | O | O | O | O | S | OMe | OMe |
| 3063a-l | Y*AE | S | O | O | O | O | S | H | OMe |
| 3064a-l | Y*AE | S | O | O | O | O | S | F | OMe |
| 3065a-l | Y*AE | S | O | O | O | O | S | OEt | OMe |
| 3066a-l | Y*AE | S | S | O | O | O | S | OH | OMe |
| 3067a-l | Y*AE | S | S | O | O | O | S | OMe | OMe |
| 3068a-l | Y*AE | S | S | O | O | O | S | H | OMe |
| 3069a-l | Y*AE | S | S | O | O | O | S | F | OMe |
| 3070a-l | Y*AE | S | S | O | O | O | S | OEt | OMe |
| 3071a-l | Y*AE | O | O | S | O | S | O | OH | OMe |
| 3072a-l | Y*AE | O | O | S | O | S | O | OMe | OMe |
| 3073a-l | Y*AE | O | O | S | O | S | O | H | OMe |
| 3074a-l | Y*AE | O | O | S | O | S | O | F | OMe |
| 3075a-l | Y*AE | O | O | S | O | S | O | OEt | OMe |
| 3076a-l | Y*AE | S | O | S | O | S | O | OH | OMe |
| 3077a-l | Y*AE | S | O | S | O | S | O | OMe | OMe |
| 3078a-l | Y*AE | S | O | S | O | S | O | H | OMe |
| 3079a-l | Y*AE | S | O | S | O | S | O | F | OMe |
| 3080a-l | Y*AE | S | O | S | O | S | O | OEt | OMe |
| 3081a-l | Y*AE | S | S | S | O | S | O | OH | OMe |
| 3082a-l | Y*AE | S | S | S | O | S | O | OMe | OMe |
| 3083a-l | Y*AE | S | S | S | O | S | O | H | OMe |
| 3084a-l | Y*AE | S | S | S | O | S | O | F | OMe |
| 3085a-l | Y*AE | S | S | S | O | S | O | OEt | OMe |
| 3086a-l | Y*AE | O | O | O | S | O | S | OH | OMe |
| 3087a-l | Y*AE | O | O | O | S | O | S | OMe | OMe |
| 3088a-l | Y*AE | O | O | O | S | O | S | H | OMe |
| 3089a-l | Y*AE | O | O | O | S | O | S | F | OMe |
| 3090a-l | Y*AE | O | O | O | S | O | S | OEt | OMe |
| 3091a-l | Y*AE | S | O | O | S | O | S | OH | OMe |
| 3092a-l | Y*AE | S | O | O | S | O | S | OMe | OMe |
| 3093a-l | Y*AE | S | O | O | S | O | S | H | OMe |
| 3094a-l | Y*AE | S | O | O | S | O | S | F | OMe |
| 3095a-l | Y*AE | S | O | O | S | O | S | OEt | OMe |
| 3096a-l | Y*AE | S | S | O | S | O | S | OH | OMe |
| 3097a-l | Y*AE | S | S | O | S | O | S | OMe | OMe |
| 3098a-l | Y*AE | S | S | O | S | O | S | H | OMe |
| 3099a-l | Y*AE | S | S | O | S | O | S | F | OMe |
| 3100a-l | Y*AE | S | S | O | S | O | S | OEt | OMe |
| 3101a-l | Y*EG | O | O | O | O | O | O | OH | OH |
| 3102a-l | Y*EG | O | O | O | O | O | O | OMe | OH |
| 3103a-l | Y*EG | O | O | O | O | O | O | H | OH |
| 3104a-l | Y*EG | O | O | O | O | O | O | F | OH |
| 3105a-l | Y*EG | O | O | O | O | O | O | OEt | OH |
| 3106a-l | Y*EG | S | O | O | O | O | O | OH | OH |
| 3107a-l | Y*EG | S | O | O | O | O | O | OMe | OH |
| 3108a-l | Y*EG | S | O | O | O | O | O | H | OH |
| 3109a-l | Y*EG | S | O | O | O | O | O | F | OH |
| 3110a-l | Y*EG | S | O | O | O | O | O | OEt | OH |
| 3111a-l | Y*EG | S | S | O | O | O | O | OH | OH |
| 3112a-l | Y*EG | S | S | O | O | O | O | OMe | OH |
| 3113a-l | Y*EG | S | S | O | O | O | O | H | OH |
| 3114a-l | Y*EG | S | S | O | O | O | O | F | OH |
| 3115a-l | Y*EG | S | S | O | O | O | O | OEt | OH |
| 3116a-l | Y*EG | O | O | S | O | O | O | OH | OH |
| 3117a-l | Y*EG | O | O | S | O | O | O | OMe | OH |
| 3118a-l | Y*EG | O | O | S | O | O | O | H | OH |
| 3119a-l | Y*EG | O | O | S | O | O | O | F | OH |
| 3120a-l | Y*EG | O | O | S | O | O | O | OEt | OH |
| 3121a-l | Y*EG | S | O | S | O | O | O | OH | OH |
| 3122a-l | Y*EG | S | O | S | O | O | O | OMe | OH |
| 3123a-l | Y*EG | S | O | S | O | O | O | H | OH |
| 3124a-l | Y*EG | S | O | S | O | O | O | F | OH |
| 3125a-l | Y*EG | S | O | S | O | O | O | OEt | OH |
| 3126a-l | Y*EG | S | S | S | O | O | O | OH | OH |
| 3127a-l | Y*EG | S | S | S | O | O | O | OMe | OH |
| 3128a-l | Y*EG | S | S | S | O | O | O | H | OH |
| 3129a-l | Y*EG | S | S | S | O | O | O | F | OH |
| 3130a-l | Y*EG | S | S | S | O | O | O | OEt | OH |
| 3131a-l | Y*EG | O | O | S | S | O | O | OH | OH |
| 3132a-l | Y*EG | O | O | S | S | O | O | OMe | OH |
| 3133a-l | Y*EG | O | O | S | S | O | O | H | OH |
| 3134a-l | Y*EG | O | O | S | S | O | O | F | OH |
| 3135a-l | Y*EG | O | O | S | S | O | O | OEt | OH |
| 3136a-l | Y*EG | S | O | S | S | O | O | OH | OH |
| 3137a-l | Y*EG | S | O | S | S | O | O | OMe | OH |
| 3138a-l | Y*EG | S | O | S | S | O | O | H | OH |
| 3139a-l | Y*EG | S | O | S | S | O | O | F | OH |
| 3140a-l | Y*EG | S | O | S | S | O | O | OEt | OH |
| 3141a-l | Y*EG | S | S | S | S | O | O | OH | OH |
| 3142a-l | Y*EG | S | S | S | S | O | O | OMe | OH |
| 3143a-l | Y*EG | S | S | S | S | O | O | H | OH |
| 3144a-l | Y*EG | S | S | S | S | O | O | F | OH |
| 3145a-l | Y*EG | S | S | S | S | O | O | OEt | OH |
| 3146a-l | Y*EG | O | O | S | S | S | O | OH | OH |
| 3147a-l | Y*EG | O | O | S | S | S | O | OMe | OH |
| 3148a-l | Y*EG | O | O | S | S | S | O | H | OH |
| 3149a-l | Y*EG | O | O | S | S | S | O | F | OH |
| 3150a-l | Y*EG | O | O | S | S | S | O | OEt | OH |
| 3151a-l | Y*EG | S | O | S | S | S | O | OH | OH |
| 3152a-l | Y*EG | S | O | S | S | S | O | OMe | OH |
| 3153a-l | Y*EG | S | O | S | S | S | O | H | OH |
| 3154a-l | Y*EG | S | O | S | S | S | O | F | OH |
| 3155a-l | Y*EG | S | O | S | S | S | O | OEt | OH |
| 3156a-l | Y*EG | S | S | S | S | S | O | OH | OH |
| 3157a-l | Y*EG | S | S | S | S | S | O | OMe | OH |
| 3158a-l | Y*EG | S | S | S | S | S | O | H | OH |
| 3159a-l | Y*EG | S | S | S | S | S | O | F | OH |
| 3160a-l | Y*EG | S | S | S | S | S | O | OEt | OH |
| 3161a-l | Y*EG | O | O | S | S | S | S | OH | OH |
| 3162a-l | Y*EG | O | O | S | S | S | S | OMe | OH |
| 3163a-l | Y*EG | O | O | S | S | S | S | H | OH |
| 3164a-l | Y*EG | O | O | S | S | S | S | F | OH |
| 3165a-l | Y*EG | O | O | S | S | S | S | OEt | OH |
| 3166a-l | Y*EG | S | O | S | S | S | S | OH | OH |
| 3167a-l | Y*EG | S | O | S | S | S | S | OMe | OH |
| 3168a-l | Y*EG | S | O | S | S | S | S | H | OH |
| 3169a-l | Y*EG | S | O | S | S | S | S | F | OH |
| 3170a-l | Y*EG | S | O | S | S | S | S | OEt | OH |
| 3171a-l | Y*EG | S | S | S | S | S | S | OH | OH |
| 3172a-l | Y*EG | S | S | S | S | S | S | OMe | OH |
| 3173a-l | Y*EG | S | S | S | S | S | S | H | OH |
| 3174a-l | Y*EG | S | S | S | S | S | S | F | OH |
| 3175a-l | Y*EG | S | S | S | S | S | S | OEt | OH |
| 3176a-l | Y*EG | O | O | O | S | S | S | OH | OH |
| 3177a-l | Y*EG | O | O | O | S | S | S | OMe | OH |
| 3178a-l | Y*EG | O | O | O | S | S | S | H | OH |
| 3179a-l | Y*EG | O | O | O | S | S | S | F | OH |
| 3180a-l | Y*EG | O | O | O | S | S | S | OEt | OH |
| 3181a-l | Y*EG | S | O | O | S | S | S | OH | OH |
| 3182a-l | Y*EG | S | O | O | S | S | S | OMe | OH |
| 3183a-l | Y*EG | S | O | O | S | S | S | H | OH |
| 3184a-l | Y*EG | S | O | O | S | S | S | F | OH |
| 3185a-l | Y*EG | S | O | O | S | S | S | OEt | OH |
| 3186a-l | Y*EG | S | S | O | S | S | S | OH | OH |
| 3187a-l | Y*EG | S | S | O | S | S | S | OMe | OH |
| 3188a-l | Y*EG | S | S | O | S | S | S | H | OH |
| 3189a-l | Y*EG | S | S | O | S | S | S | F | OH |
| 3190a-l | Y*EG | S | S | O | S | S | S | OEt | OH |
| 3191a-l | Y*EG | O | O | O | O | S | S | OH | OH |
| 3192a-l | Y*EG | O | O | O | O | S | S | OMe | OH |
| 3193a-l | Y*EG | O | O | O | O | S | S | H | OH |
| 3194a-l | Y*EG | O | O | O | O | S | S | F | OH |
| 3195a-l | Y*EG | O | O | O | O | S | S | OEt | OH |
| 3196a-l | Y*EG | S | O | O | O | S | S | OH | OH |
| 3197a-l | Y*EG | S | O | O | O | S | S | OMe | OH |
| 3198a-l | Y*EG | S | O | O | O | S | S | H | OH |
| 3199a-l | Y*EG | S | O | O | O | S | S | F | OH |
| 3200a-l | Y*EG | S | O | O | O | S | S | OEt | OH |
| 3201a-l | Y*EG | S | S | O | O | S | S | OH | OH |
| 3202a-l | Y*EG | S | S | O | O | S | S | OMe | OH |
| 3203a-l | Y*EG | S | S | O | O | S | S | H | OH |
| 3204a-l | Y*EG | S | S | O | O | S | S | F | OH |
| 3205a-l | Y*EG | S | S | O | O | S | S | OEt | OH |
| 3206a-l | Y*EG | O | O | O | O | O | S | OH | OH |
| 3207a-l | Y*EG | O | O | O | O | O | S | OMe | OH |
| 3208a-l | Y*EG | O | O | O | O | O | S | H | OH |
| 3209a-l | Y*EG | O | O | O | O | O | S | F | OH |
| 3210a-l | Y*EG | O | O | O | O | O | S | OEt | OH |
| 3211a-l | Y*EG | S | O | O | O | O | S | OH | OH |
| 3212a-l | Y*EG | S | O | O | O | O | S | OMe | OH |
| 3213a-l | Y*EG | S | O | O | O | O | S | H | OH |
| 3214a-l | Y*EG | S | O | O | O | O | S | F | OH |
| 3215a-l | Y*EG | S | O | O | O | O | S | OEt | OH |
| 3216a-l | Y*EG | S | S | O | O | O | S | OH | OH |
| 3217a-l | Y*EG | S | S | O | O | O | S | OMe | OH |
| 3218a-l | Y*EG | S | S | O | O | O | S | H | OH |
| 3219a-l | Y*EG | S | S | O | O | O | S | F | OH |
| 3220a-l | Y*EG | S | S | O | O | O | S | OEt | OH |
| 3221a-l | Y*EG | O | O | S | O | S | O | OH | OH |
| 3222a-l | Y*EG | O | O | S | O | S | O | OMe | OH |
| 3223a-l | Y*EG | O | O | S | O | S | O | H | OH |
| 3224a-l | Y*EG | O | O | S | O | S | O | F | OH |
| 3225a-l | Y*EG | O | O | S | O | S | O | OEt | OH |
| 3226a-l | Y*EG | S | O | S | O | S | O | OH | OH |
| 3227a-l | Y*EG | S | O | S | O | S | O | OMe | OH |
| 3228a-l | Y*EG | S | O | S | O | S | O | H | OH |
| 3229a-l | Y*EG | S | O | S | O | S | O | F | OH |
| 3230a-l | Y*EG | S | O | S | O | S | O | OEt | OH |
| 3231a-l | Y*EG | S | S | S | O | S | O | OH | OH |
| 3232a-l | Y*EG | S | S | S | O | S | O | OMe | OH |
| 3233a-l | Y*EG | S | S | S | O | S | O | H | OH |
| 3234a-l | Y*EG | S | S | S | O | S | O | F | OH |
| 3235a-l | Y*EG | S | S | S | O | S | O | OEt | OH |
| 3236a-l | Y*EG | O | O | O | S | O | S | OH | OH |
| 3237a-l | Y*EG | O | O | O | S | O | S | OMe | OH |
| 3238a-l | Y*EG | O | O | O | S | O | S | H | OH |
| 3239a-l | Y*EG | O | O | O | S | O | S | F | OH |
| 3240a-l | Y*EG | O | O | O | S | O | S | OEt | OH |
| 3241a-l | Y*EG | S | O | O | S | O | S | OH | OH |
| 3242a-l | Y*EG | S | O | O | S | O | S | OMe | OH |
| 3243a-l | Y*EG | S | O | O | S | O | S | H | OH |
| 3244a-l | Y*EG | S | O | O | S | O | S | F | OH |
| 3245a-l | Y*EG | S | O | O | S | O | S | OEt | OH |
| 3246a-l | Y*EG | S | S | O | S | O | S | OH | OH |
| 3247a-l | Y*EG | S | S | O | S | O | S | OMe | OH |
| 3248a-l | Y*EG | S | S | O | S | O | S | H | OH |
| 3249a-l | Y*EG | S | S | O | S | O | S | F | OH |
| 3250a-l | Y*EG | S | S | O | S | O | S | OEt | OH |
| 3251a-l | Y*EG | O | O | O | O | O | O | OH | OMe |
| 3252a-l | Y*EG | O | O | O | O | O | O | OMe | OMe |
| 3253a-l | Y*EG | O | O | O | O | O | O | H | OMe |
| 3254a-l | Y*EG | O | O | O | O | O | O | F | OMe |
| 3255a-l | Y*EG | O | O | O | O | O | O | OEt | OMe |
| 3256a-l | Y*EG | S | O | O | O | O | O | OH | OMe |
| 3257a-l | Y*EG | S | O | O | O | O | O | OMe | OMe |
| 3258a-l | Y*EG | S | O | O | O | O | O | H | OMe |
| 3259a-l | Y*EG | S | O | O | O | O | O | F | OMe |
| 3260a-l | Y*EG | S | O | O | O | O | O | OEt | OMe |
| 3261a-l | Y*EG | S | S | O | O | O | O | OH | OMe |
| 3262a-l | Y*EG | S | S | O | O | O | O | OMe | OMe |
| 3263a-l | Y*EG | S | S | O | O | O | O | H | OMe |
| 3264a-l | Y*EG | S | S | O | O | O | O | F | OMe |
| 3265a-l | Y*EG | S | S | O | O | O | O | OEt | OMe |
| 3266a-l | Y*EG | O | O | S | O | O | O | OH | OMe |
| 3267a-l | Y*EG | O | O | S | O | O | O | OMe | OMe |
| 3268a-l | Y*EG | O | O | S | O | O | O | H | OMe |
| 3269a-l | Y*EG | O | O | S | O | O | O | F | OMe |
| 3270a-l | Y*EG | O | O | S | O | O | O | OEt | OMe |
| 3271a-l | Y*EG | S | O | S | O | O | O | OH | OMe |
| 3272a-l | Y*EG | S | O | S | O | O | O | OMe | OMe |
| 3273a-l | Y*EG | S | O | S | O | O | O | H | OMe |
| 3274a-l | Y*EG | S | O | S | O | O | O | F | OMe |
| 3275a-l | Y*EG | S | O | S | O | O | O | OEt | OMe |
| 3276a-l | Y*EG | S | S | S | O | O | O | OH | OMe |
| 3277a-l | Y*EG | S | S | S | O | O | O | OMe | OMe |
| 3278a-l | Y*EG | S | S | S | O | O | O | H | OMe |
| 3279a-l | Y*EG | S | S | S | O | O | O | F | OMe |
| 3280a-l | Y*EG | S | S | S | O | O | O | OEt | OMe |
| 3281a-l | Y*EG | O | O | S | S | O | O | OH | OMe |
| 3282a-l | Y*EG | O | O | S | S | O | O | OMe | OMe |
| 3283a-l | Y*EG | O | O | S | S | O | O | H | OMe |
| 3284a-l | Y*EG | O | O | S | S | O | O | F | OMe |
| 3285a-l | Y*EG | O | O | S | S | O | O | OEt | OMe |
| 3286a-l | Y*EG | S | O | S | S | O | O | OH | OMe |
| 3287a-l | Y*EG | S | O | S | S | O | O | OMe | OMe |
| 3288a-l | Y*EG | S | O | S | S | O | O | H | OMe |
| 3289a-l | Y*EG | S | O | S | S | O | O | F | OMe |
| 3290a-l | Y*EG | S | O | S | S | O | O | OEt | OMe |
| 3291a-l | Y*EG | S | S | S | S | O | O | OH | OMe |
| 3292a-l | Y*EG | S | S | S | S | O | O | OMe | OMe |
| 3293a-l | Y*EG | S | S | S | S | O | O | H | OMe |
| 3294a-l | Y*EG | S | S | S | S | O | O | F | OMe |
| 3295a-l | Y*EG | S | S | S | S | O | O | OEt | OMe |
| 3296a-l | Y*EG | O | O | S | S | S | O | OH | OMe |
| 3297a-l | Y*EG | O | O | S | S | S | O | OMe | OMe |
| 3298a-l | Y*EG | O | O | S | S | S | O | H | OMe |
| 3299a-l | Y*EG | O | O | S | S | S | O | F | OMe |
| 3300a-l | Y*EG | O | O | S | S | S | O | OEt | OMe |
| 3301a-l | Y*EG | S | O | S | S | S | O | OH | OMe |
| 3302a-l | Y*EG | S | O | S | S | S | O | OMe | OMe |
| 3303a-l | Y*EG | S | O | S | S | S | O | H | OMe |
| 3304a-l | Y*EG | S | O | S | S | S | O | F | OMe |
| 3305a-l | Y*EG | S | O | S | S | S | O | OEt | OMe |
| 3306a-l | Y*EG | S | S | S | S | S | O | OH | OMe |
| 3307a-l | Y*EG | S | S | S | S | S | O | OMe | OMe |
| 3308a-l | Y*EG | S | S | S | S | S | O | H | OMe |
| 3309a-l | Y*EG | S | S | S | S | S | O | F | OMe |
| 3310a-l | Y*EG | S | S | S | S | S | O | OEt | OMe |
| 3311a-l | Y*EG | O | O | S | S | S | S | OH | OMe |
| 3312a-l | Y*EG | O | O | S | S | S | S | OMe | OMe |
| 3313a-l | Y*EG | O | O | S | S | S | S | H | OMe |
| 3314a-l | Y*EG | O | O | S | S | S | S | F | OMe |
| 3315a-l | Y*EG | O | O | S | S | S | S | OEt | OMe |
| 3316a-l | Y*EG | S | O | S | S | S | S | OH | OMe |
| 3317a-l | Y*EG | S | O | S | S | S | S | OMe | OMe |
| 3318a-l | Y*EG | S | O | S | S | S | S | H | OMe |
| 3319a-l | Y*EG | S | O | S | S | S | S | F | OMe |
| 3320a-l | Y*EG | S | O | S | S | S | S | OEt | OMe |
| 3321a-l | Y*EG | S | S | S | S | S | S | OH | OMe |
| 3322a-l | Y*EG | S | S | S | S | S | S | OMe | OMe |
| 3323a-l | Y*EG | S | S | S | S | S | S | H | OMe |
| 3324a-l | Y*EG | S | S | S | S | S | S | F | OMe |
| 3325a-l | Y*EG | S | S | S | S | S | S | OEt | OMe |
| 3326a-l | Y*EG | O | O | O | S | S | S | OH | OMe |
| 3327a-l | Y*EG | O | O | O | S | S | S | OMe | OMe |
| 3328a-l | Y*EG | O | O | O | S | S | S | H | OMe |
| 3329a-l | Y*EG | O | O | O | S | S | S | F | OMe |
| 3330a-l | Y*EG | O | O | O | S | S | S | OEt | OMe |
| 3331a-l | Y*EG | S | O | O | S | S | S | OH | OMe |
| 3332a-l | Y*EG | S | O | O | S | S | S | OMe | OMe |
| 3333a-l | Y*EG | S | O | O | S | S | S | H | OMe |
| 3334a-l | Y*EG | S | O | O | S | S | S | F | OMe |
| 3335a-l | Y*EG | S | O | O | S | S | S | OEt | OMe |
| 3336a-l | Y*EG | S | S | O | S | S | S | OH | OMe |
| 3337a-l | Y*EG | S | S | O | S | S | S | OMe | OMe |
| 3338a-l | Y*EG | S | S | O | S | S | S | H | OMe |
| 3339a-l | Y*EG | S | S | O | S | S | S | F | OMe |
| 3340a-l | Y*EG | S | S | O | S | S | S | OEt | OMe |
| 3341a-l | Y*EG | O | O | O | O | S | S | OH | OMe |
| 3342a-l | Y*EG | O | O | O | O | S | S | OMe | OMe |
| 3343a-l | Y*EG | O | O | O | O | S | S | H | OMe |
| 3344a-l | Y*EG | O | O | O | O | S | S | F | OMe |
| 3345a-l | Y*EG | O | O | O | O | S | S | OEt | OMe |
| 3346a-l | Y*EG | S | O | O | O | S | S | OH | OMe |
| 3347a-l | Y*EG | S | O | O | O | S | S | OMe | OMe |
| 3348a-l | Y*EG | S | O | O | O | S | S | H | OMe |
| 3349a-l | Y*EG | S | O | O | O | S | S | F | OMe |
| 3350a-l | Y*EG | S | O | O | O | S | S | OEt | OMe |
| 3351a-l | Y*EG | S | S | O | O | S | S | OH | OMe |
| 3352a-l | Y*EG | S | S | O | O | S | S | OMe | OMe |
| 3353a-l | Y*EG | S | S | O | O | S | S | H | OMe |
| 3354a-l | Y*EG | S | S | O | O | S | S | F | OMe |
| 3355a-l | Y*EG | S | S | O | O | S | S | OEt | OMe |
| 3356a-l | Y*EG | O | O | O | O | O | S | OH | OMe |
| 3357a-l | Y*EG | O | O | O | O | O | S | OMe | OMe |
| 3358a-l | Y*EG | O | O | O | O | O | S | H | OMe |
| 3359a-l | Y*EG | O | O | O | O | O | S | F | OMe |
| 3360a-l | Y*EG | O | O | O | O | O | S | OEt | OMe |
| 3361a-l | Y*EG | S | O | O | O | O | S | OH | OMe |
| 3362a-l | Y*EG | S | O | O | O | O | S | OMe | OMe |
| 3363a-l | Y*EG | S | O | O | O | O | S | H | OMe |
| 3364a-l | Y*EG | S | O | O | O | O | S | F | OMe |
| 3365a-l | Y*EG | S | O | O | O | O | S | OEt | OMe |
| 3366a-l | Y*EG | S | S | O | O | O | S | OH | OMe |
| 3367a-l | Y*EG | S | S | O | O | O | S | OMe | OMe |
| 3368a-l | Y*EG | S | S | O | O | O | S | H | OMe |
| 3369a-l | Y*EG | S | S | O | O | O | S | F | OMe |
| 3370a-l | Y*EG | S | S | O | O | O | S | OEt | OMe |
| 3371a-l | Y*EG | O | O | S | O | S | O | OH | OMe |
| 3372a-l | Y*EG | O | O | S | O | S | O | OMe | OMe |
| 3373a-l | Y*EG | O | O | S | O | S | O | H | OMe |
| 3374a-l | Y*EG | O | O | S | O | S | O | F | OMe |
| 3375a-l | Y*EG | O | O | S | O | S | O | OEt | OMe |
| 3376a-l | Y*EG | S | O | S | O | S | O | OH | OMe |
| 3377a-l | Y*EG | S | O | S | O | S | O | OMe | OMe |
| 3378a-l | Y*EG | S | O | S | O | S | O | H | OMe |
| 3379a-l | Y*EG | S | O | S | O | S | O | F | OMe |
| 3380a-l | Y*EG | S | O | S | O | S | O | OEt | OMe |
| 3381a-l | Y*EG | S | S | S | O | S | O | OH | OMe |
| 3382a-l | Y*EG | S | S | S | O | S | O | OMe | OMe |
| 3383a-l | Y*EG | S | S | S | O | S | O | H | OMe |
| 3384a-l | Y*EG | S | S | S | O | S | O | F | OMe |
| 3385a-l | Y*EG | S | S | S | O | S | O | OEt | OMe |
| 3386a-l | Y*EG | O | O | O | S | O | S | OH | OMe |
| 3387a-l | Y*EG | O | O | O | S | O | S | OMe | OMe |
| 3388a-l | Y*EG | O | O | O | S | O | S | H | OMe |
| 3389a-l | Y*EG | O | O | O | S | O | S | F | OMe |
| 3390a-l | Y*EG | O | O | O | S | O | S | OEt | OMe |
| 3391a-l | Y*EG | S | O | O | S | O | S | OH | OMe |
| 3392a-l | Y*EG | S | O | O | S | O | S | OMe | OMe |
| 3393a-l | Y*EG | S | O | O | S | O | S | H | OMe |
| 3394a-l | Y*EG | S | O | O | S | O | S | F | OMe |
| 3395a-l | Y*EG | S | O | O | S | O | S | OEt | OMe |
| 3396a-l | Y*EG | S | S | O | S | O | S | OH | OMe |
| 3397a-l | Y*EG | S | S | O | S | O | S | OMe | OMe |
| 3398a-l | Y*EG | S | S | O | S | O | S | H | OMe |
| 3399a-l | Y*EG | S | S | O | S | O | S | F | OMe |
| 3400a-l | Y*EG | S | S | O | S | O | S | OEt | OMe |
| 3401a-l | Y*EA | O | O | O | O | O | O | OH | OH |
| 3402a-l | Y*EA | O | O | O | O | O | O | OMe | OH |
| 3403a-l | Y*EA | O | O | O | O | O | O | H | OH |
| 3404a-l | Y*EA | O | O | O | O | O | O | F | OH |
| 3405a-l | Y*EA | O | O | O | O | O | O | OEt | OH |
| 3406a-l | Y*EA | S | O | O | O | O | O | OH | OH |
| 3407a-l | Y*EA | S | O | O | O | O | O | OMe | OH |
| 3408a-l | Y*EA | S | O | O | O | O | O | H | OH |
| 3409a-l | Y*EA | S | O | O | O | O | O | F | OH |
| 3410a-l | Y*EA | S | O | O | O | O | O | OEt | OH |
| 3411a-l | Y*EA | S | S | O | O | O | O | OH | OH |
| 3412a-l | Y*EA | S | S | O | O | O | O | OMe | OH |
| 3413a-l | Y*EA | S | S | O | O | O | O | H | OH |
| 3414a-l | Y*EA | S | S | O | O | O | O | F | OH |
| 3415a-l | Y*EA | S | S | O | O | O | O | OEt | OH |
| 3416a-l | Y*EA | O | O | S | O | O | O | OH | OH |
| 3417a-l | Y*EA | O | O | S | O | O | O | OMe | OH |
| 3418a-l | Y*EA | O | O | S | O | O | O | H | OH |
| 3419a-l | Y*EA | O | O | S | O | O | O | F | OH |
| 3420a-l | Y*EA | O | O | S | O | O | O | OEt | OH |
| 3421a-l | Y*EA | S | O | S | O | O | O | OH | OH |
| 3422a-l | Y*EA | S | O | S | O | O | O | OMe | OH |
| 3423a-l | Y*EA | S | O | S | O | O | O | H | OH |
| 3424a-l | Y*EA | S | O | S | O | O | O | F | OH |
| 3425a-l | Y*EA | S | O | S | O | O | O | OEt | OH |
| 3426a-l | Y*EA | S | S | S | O | O | O | OH | OH |
| 3427a-l | Y*EA | S | S | S | O | O | O | OMe | OH |
| 3428a-l | Y*EA | S | S | S | O | O | O | H | OH |
| 3429a-l | Y*EA | S | S | S | O | O | O | F | OH |
| 3430a-l | Y*EA | S | S | S | O | O | O | OEt | OH |
| 3431a-l | Y*EA | O | O | S | S | O | O | OH | OH |
| 3432a-l | Y*EA | O | O | S | S | O | O | OMe | OH |
| 3433a-l | Y*EA | O | O | S | S | O | O | H | OH |
| 3434a-l | Y*EA | O | O | S | S | O | O | F | OH |
| 3435a-l | Y*EA | O | O | S | S | O | O | OEt | OH |
| 3436a-l | Y*EA | S | O | S | S | O | O | OH | OH |
| 3437a-l | Y*EA | S | O | S | S | O | O | OMe | OH |
| 3438a-l | Y*EA | S | O | S | S | O | O | H | OH |
| 3439a-l | Y*EA | S | O | S | S | O | O | F | OH |
| 3440a-l | Y*EA | S | O | S | S | O | O | OEt | OH |
| 3441a-l | Y*EA | S | S | S | S | O | O | OH | OH |
| 3442a-l | Y*EA | S | S | S | S | O | O | OMe | OH |
| 3443a-l | Y*EA | S | S | S | S | O | O | H | OH |
| 3444a-l | Y*EA | S | S | S | S | O | O | F | OH |
| 3445a-l | Y*EA | S | S | S | S | O | O | OEt | OH |
| 3446a-l | Y*EA | O | O | S | S | S | O | OH | OH |
| 3447a-l | Y*EA | O | O | S | S | S | O | OMe | OH |
| 3448a-l | Y*EA | O | O | S | S | S | O | H | OH |
| 3449a-l | Y*EA | O | O | S | S | S | O | F | OH |
| 3450a-l | Y*EA | O | O | S | S | S | O | OEt | OH |
| 3451a-l | Y*EA | S | O | S | S | S | O | OH | OH |
| 3452a-l | Y*EA | S | O | S | S | S | O | OMe | OH |
| 3453a-l | Y*EA | S | O | S | S | S | O | H | OH |
| 3454a-l | Y*EA | S | O | S | S | S | O | F | OH |
| 3455a-l | Y*EA | S | O | S | S | S | O | OEt | OH |
| 3456a-l | Y*EA | S | S | S | S | S | O | OH | OH |
| 3457a-l | Y*EA | S | S | S | S | S | O | OMe | OH |
| 3458a-l | Y*EA | S | S | S | S | S | O | H | OH |
| 3459a-l | Y*EA | S | S | S | S | S | O | F | OH |
| 3460a-l | Y*EA | S | S | S | S | S | O | OEt | OH |
| 3461a-l | Y*EA | O | O | S | S | S | S | OH | OH |
| 3462a-l | Y*EA | O | O | S | S | S | S | OMe | OH |
| 3463a-l | Y*EA | O | O | S | S | S | S | H | OH |
| 3464a-l | Y*EA | O | O | S | S | S | S | F | OH |
| 3465a-l | Y*EA | O | O | S | S | S | S | OEt | OH |
| 3466a-l | Y*EA | S | O | S | S | S | S | OH | OH |
| 3467a-l | Y*EA | S | O | S | S | S | S | OMe | OH |
| 3468a-l | Y*EA | S | O | S | S | S | S | H | OH |
| 3469a-l | Y*EA | S | O | S | S | S | S | F | OH |
| 3470a-l | Y*EA | S | O | S | S | S | S | OEt | OH |
| 3471a-l | Y*EA | S | S | S | S | S | S | OH | OH |
| 3472a-l | Y*EA | S | S | S | S | S | S | OMe | OH |
| 3473a-l | Y*EA | S | S | S | S | S | S | H | OH |
| 3474a-l | Y*EA | S | S | S | S | S | S | F | OH |
| 3475a-l | Y*EA | S | S | S | S | S | S | OEt | OH |
| 3476a-l | Y*EA | O | O | O | S | S | S | OH | OH |
| 3477a-l | Y*EA | O | O | O | S | S | S | OMe | OH |
| 3478a-l | Y*EA | O | O | O | S | S | S | H | OH |
| 3479a-l | Y*EA | O | O | O | S | S | S | F | OH |
| 3480a-l | Y*EA | O | O | O | S | S | S | OEt | OH |
| 3481a-l | Y*EA | S | O | O | S | S | S | OH | OH |
| 3482a-l | Y*EA | S | O | O | S | S | S | OMe | OH |
| 3483a-l | Y*EA | S | O | O | S | S | S | H | OH |
| 3484a-l | Y*EA | S | O | O | S | S | S | F | OH |
| 3485a-l | Y*EA | S | O | O | S | S | S | OEt | OH |
| 3486a-l | Y*EA | S | S | O | S | S | S | OH | OH |
| 3487a-l | Y*EA | S | S | O | S | S | S | OMe | OH |
| 3488a-l | Y*EA | S | S | O | S | S | S | H | OH |
| 3489a-l | Y*EA | S | S | O | S | S | S | F | OH |
| 3490a-l | Y*EA | S | S | O | S | S | S | OEt | OH |
| 3491a-l | Y*EA | O | O | O | O | S | S | OH | OH |
| 3492a-l | Y*EA | O | O | O | O | S | S | OMe | OH |
| 3493a-l | Y*EA | O | O | O | O | S | S | H | OH |
| 3494a-l | Y*EA | O | O | O | O | S | S | F | OH |
| 3495a-l | Y*EA | O | O | O | O | S | S | OEt | OH |
| 3496a-l | Y*EA | S | O | O | O | S | S | OH | OH |
| 3497a-l | Y*EA | S | O | O | O | S | S | OMe | OH |
| 3498a-l | Y*EA | S | O | O | O | S | S | H | OH |
| 3499a-l | Y*EA | S | O | O | O | S | S | F | OH |
| 3500a-l | Y*EA | S | O | O | O | S | S | OEt | OH |
| 3501a-l | Y*EA | S | S | O | O | S | S | OH | OH |
| 3502a-l | Y*EA | S | S | O | O | S | S | OMe | OH |
| 3503a-l | Y*EA | S | S | O | O | S | S | H | OH |
| 3504a-l | Y*EA | S | S | O | O | S | S | F | OH |
| 3505a-l | Y*EA | S | S | O | O | S | S | OEt | OH |
| 3506a-l | Y*EA | O | O | O | O | O | S | OH | OH |
| 3507a-l | Y*EA | O | O | O | O | O | S | OMe | OH |
| 3508a-l | Y*EA | O | O | O | O | O | S | H | OH |
| 3509a-l | Y*EA | O | O | O | O | O | S | F | OH |
| 3510a-l | Y*EA | O | O | O | O | O | S | OEt | OH |
| 3511a-l | Y*EA | S | O | O | O | O | S | OH | OH |
| 3512a-l | Y*EA | S | O | O | O | O | S | OMe | OH |
| 3513a-l | Y*EA | S | O | O | O | O | S | H | OH |
| 3514a-l | Y*EA | S | O | O | O | O | S | F | OH |
| 3515a-l | Y*EA | S | O | O | O | O | S | OEt | OH |
| 3516a-l | Y*EA | S | S | O | O | O | S | OH | OH |
| 3517a-l | Y*EA | S | S | O | O | O | S | OMe | OH |
| 3518a-l | Y*EA | S | S | O | O | O | S | H | OH |
| 3519a-l | Y*EA | S | S | O | O | O | S | F | OH |
| 3520a-l | Y*EA | S | S | O | O | O | S | OEt | OH |
| 3521a-l | Y*EA | O | O | S | O | S | O | OH | OH |
| 3522a-l | Y*EA | O | O | S | O | S | O | OMe | OH |
| 3523a-l | Y*EA | O | O | S | O | S | O | H | OH |
| 3524a-l | Y*EA | O | O | S | O | S | O | F | OH |
| 3525a-l | Y*EA | O | O | S | O | S | O | OEt | OH |
| 3526a-l | Y*EA | S | O | S | O | S | O | OH | OH |
| 3527a-l | Y*EA | S | O | S | O | S | O | OMe | OH |
| 3528a-l | Y*EA | S | O | S | O | S | O | H | OH |
| 3529a-l | Y*EA | S | O | S | O | S | O | F | OH |
| 3530a-l | Y*EA | S | O | S | O | S | O | OEt | OH |
| 3531a-l | Y*EA | S | S | S | O | S | O | OH | OH |
| 3532a-l | Y*EA | S | S | S | O | S | O | OMe | OH |
| 3533a-l | Y*EA | S | S | S | O | S | O | H | OH |
| 3534a-l | Y*EA | S | S | S | O | S | O | F | OH |
| 3535a-l | Y*EA | S | S | S | O | S | O | OEt | OH |
| 3536a-l | Y*EA | O | O | O | S | O | S | OH | OH |
| 3537a-l | Y*EA | O | O | O | S | O | S | OMe | OH |
| 3538a-l | Y*EA | O | O | O | S | O | S | H | OH |
| 3539a-l | Y*EA | O | O | O | S | O | S | F | OH |
| 3540a-l | Y*EA | O | O | O | S | O | S | OEt | OH |
| 3541a-l | Y*EA | S | O | O | S | O | S | OH | OH |
| 3542a-l | Y*EA | S | O | O | S | O | S | OMe | OH |
| 3543a-l | Y*EA | S | O | O | S | O | S | H | OH |
| 3544a-l | Y*EA | S | O | O | S | O | S | F | OH |
| 3545a-l | Y*EA | S | O | O | S | O | S | OEt | OH |
| 3546a-l | Y*EA | S | S | O | S | O | S | OH | OH |
| 3547a-l | Y*EA | S | S | O | S | O | S | OMe | OH |
| 3548a-l | Y*EA | S | S | O | S | O | S | H | OH |
| 3549a-l | Y*EA | S | S | O | S | O | S | F | OH |
| 3550a-l | Y*EA | S | S | O | S | O | S | OEt | OH |
| 3551a-l | Y*EA | O | O | O | O | O | O | OH | OMe |
| 3552a-l | Y*EA | O | O | O | O | O | O | OMe | OMe |
| 3553a-l | Y*EA | O | O | O | O | O | O | H | OMe |
| 3554a-l | Y*EA | O | O | O | O | O | O | F | OMe |
| 3555a-l | Y*EA | O | O | O | O | O | O | OEt | OMe |
| 3556a-l | Y*EA | S | O | O | O | O | O | OH | OMe |
| 3557a-l | Y*EA | S | O | O | O | O | O | OMe | OMe |
| 3558a-l | Y*EA | S | O | O | O | O | O | H | OMe |
| 3559a-l | Y*EA | S | O | O | O | O | O | F | OMe |
| 3560a-l | Y*EA | S | O | O | O | O | O | OEt | OMe |
| 3561a-l | Y*EA | S | S | O | O | O | O | OH | OMe |
| 3562a-l | Y*EA | S | S | O | O | O | O | OMe | OMe |
| 3563a-l | Y*EA | S | S | O | O | O | O | H | OMe |
| 3564a-l | Y*EA | S | S | O | O | O | O | F | OMe |
| 3565a-l | Y*EA | S | S | O | O | O | O | OEt | OMe |
| 3566a-l | Y*EA | O | O | S | O | O | O | OH | OMe |
| 3567a-l | Y*EA | O | O | S | O | O | O | OMe | OMe |
| 3568a-l | Y*EA | O | O | S | O | O | O | H | OMe |
| 3569a-l | Y*EA | O | O | S | O | O | O | F | OMe |
| 3570a-l | Y*EA | O | O | S | O | O | O | OEt | OMe |
| 3571a-l | Y*EA | S | O | S | O | O | O | OH | OMe |
| 3572a-l | Y*EA | S | O | S | O | O | O | OMe | OMe |
| 3573a-l | Y*EA | S | O | S | O | O | O | H | OMe |
| 3574a-l | Y*EA | S | O | S | O | O | O | F | OMe |
| 3575a-l | Y*EA | S | O | S | O | O | O | OEt | OMe |
| 3576a-l | Y*EA | S | S | S | O | O | O | OH | OMe |
| 3577a-l | Y*EA | S | S | S | O | O | O | OMe | OMe |
| 3578a-l | Y*EA | S | S | S | O | O | O | H | OMe |
| 3579a-l | Y*EA | S | S | S | O | O | O | F | OMe |
| 3580a-l | Y*EA | S | S | S | O | O | O | OEt | OMe |
| 3581a-l | Y*EA | O | O | S | S | O | O | OH | OMe |
| 3582a-l | Y*EA | O | O | S | S | O | O | OMe | OMe |
| 3583a-l | Y*EA | O | O | S | S | O | O | H | OMe |
| 3584a-l | Y*EA | O | O | S | S | O | O | F | OMe |
| 3585a-l | Y*EA | O | O | S | S | O | O | OEt | OMe |
| 3586a-l | Y*EA | S | O | S | S | O | O | OH | OMe |
| 3587a-l | Y*EA | S | O | S | S | O | O | OMe | OMe |
| 3588a-l | Y*EA | S | O | S | S | O | O | H | OMe |
| 3589a-l | Y*EA | S | O | S | S | O | O | F | OMe |
| 3590a-l | Y*EA | S | O | S | S | O | O | OEt | OMe |
| 3591a-l | Y*EA | S | S | S | S | O | O | OH | OMe |
| 3592a-l | Y*EA | S | S | S | S | O | O | OMe | OMe |
| 3593a-l | Y*EA | S | S | S | S | O | O | H | OMe |
| 3594a-l | Y*EA | S | S | S | S | O | O | F | OMe |
| 3595a-l | Y*EA | S | S | S | S | O | O | OEt | OMe |
| 3596a-l | Y*EA | O | O | S | S | S | O | OH | OMe |
| 3597a-l | Y*EA | O | O | S | S | S | O | OMe | OMe |
| 3598a-l | Y*EA | O | O | S | S | S | O | H | OMe |
| 3599a-l | Y*EA | O | O | S | S | S | O | F | OMe |
| 3600a-l | Y*EA | O | O | S | S | S | O | OEt | OMe |
| 3601a-l | Y*EA | S | O | S | S | S | O | OH | OMe |
| 3602a-l | Y*EA | S | O | S | S | S | O | OMe | OMe |
| 3603a-l | Y*EA | S | O | S | S | S | O | H | OMe |
| 3604a-l | Y*EA | S | O | S | S | S | O | F | OMe |
| 3605a-l | Y*EA | S | O | S | S | S | O | OEt | OMe |
| 3606a-l | Y*EA | S | S | S | S | S | O | OH | OMe |
| 3607a-l | Y*EA | S | S | S | S | S | O | OMe | OMe |
| 3608a-l | Y*EA | S | S | S | S | S | O | H | OMe |
| 3609a-l | Y*EA | S | S | S | S | S | O | F | OMe |
| 3610a-l | Y*EA | S | S | S | S | S | O | OEt | OMe |
| 3611a-l | Y*EA | O | O | S | S | S | S | OH | OMe |
| 3612a-l | Y*EA | O | O | S | S | S | S | OMe | OMe |
| 3613a-l | Y*EA | O | O | S | S | S | S | H | OMe |
| 3614a-l | Y*EA | O | O | S | S | S | S | F | OMe |
| 3615a-l | Y*EA | O | O | S | S | S | S | OEt | OMe |
| 3616a-l | Y*EA | S | O | S | S | S | S | OH | OMe |
| 3617a-l | Y*EA | S | O | S | S | S | S | OMe | OMe |
| 3618a-l | Y*EA | S | O | S | S | S | S | H | OMe |
| 3619a-l | Y*EA | S | O | S | S | S | S | F | OMe |
| 3620a-l | Y*EA | S | O | S | S | S | S | OEt | OMe |
| 3621a-l | Y*EA | S | S | S | S | S | S | OH | OMe |
| 3622a-l | Y*EA | S | S | S | S | S | S | OMe | OMe |
| 3623a-l | Y*EA | S | S | S | S | S | S | H | OMe |
| 3624a-l | Y*EA | S | S | S | S | S | S | F | OMe |
| 3625a-l | Y*EA | S | S | S | S | S | S | OEt | OMe |
| 3626a-l | Y*EA | O | O | O | S | S | S | OH | OMe |
| 3627a-l | Y*EA | O | O | O | S | S | S | OMe | OMe |
| 3628a-l | Y*EA | O | O | O | S | S | S | H | OMe |
| 3629a-l | Y*EA | O | O | O | S | S | S | F | OMe |
| 3630a-l | Y*EA | O | O | O | S | S | S | OEt | OMe |
| 3631a-l | Y*EA | S | O | O | S | S | S | OH | OMe |
| 3632a-l | Y*EA | S | O | O | S | S | S | OMe | OMe |
| 3633a-l | Y*EA | S | O | O | S | S | S | H | OMe |
| 3634a-l | Y*EA | S | O | O | S | S | S | F | OMe |
| 3635a-l | Y*EA | S | O | O | S | S | S | OEt | OMe |
| 3636a-l | Y*EA | S | S | O | S | S | S | OH | OMe |
| 3637a-l | Y*EA | S | S | O | S | S | S | OMe | OMe |
| 3638a-l | Y*EA | S | S | O | S | S | S | H | OMe |
| 3639a-l | Y*EA | S | S | O | S | S | S | F | OMe |
| 3640a-l | Y*EA | S | S | O | S | S | S | OEt | OMe |
| 3641a-l | Y*EA | O | O | O | O | S | S | OH | OMe |
| 3642a-l | Y*EA | O | O | O | O | S | S | OMe | OMe |
| 3643a-l | Y*EA | O | O | O | O | S | S | H | OMe |
| 3644a-l | Y*EA | O | O | O | O | S | S | F | OMe |
| 3645a-l | Y*EA | O | O | O | O | S | S | OEt | OMe |
| 3646a-l | Y*EA | S | O | O | O | S | S | OH | OMe |
| 3647a-l | Y*EA | S | O | O | O | S | S | OMe | OMe |
| 3648a-l | Y*EA | S | O | O | O | S | S | H | OMe |
| 3649a-l | Y*EA | S | O | O | O | S | S | F | OMe |
| 3650a-l | Y*EA | S | O | O | O | S | S | OEt | OMe |
| 3651a-l | Y*EA | S | S | O | O | S | S | OH | OMe |
| 3652a-l | Y*EA | S | S | O | O | S | S | OMe | OMe |
| 3653a-l | Y*EA | S | S | O | O | S | S | H | OMe |
| 3654a-l | Y*EA | S | S | O | O | S | S | F | OMe |
| 3655a-l | Y*EA | S | S | O | O | S | S | OEt | OMe |
| 3656a-l | Y*EA | O | O | O | O | O | S | OH | OMe |
| 3657a-l | Y*EA | O | O | O | O | O | S | OMe | OMe |
| 3658a-l | Y*EA | O | O | O | O | O | S | H | OMe |
| 3659a-l | Y*EA | O | O | O | O | O | S | F | OMe |
| 3660a-l | Y*EA | O | O | O | O | O | S | OEt | OMe |
| 3661a-l | Y*EA | S | O | O | O | O | S | OH | OMe |
| 3662a-l | Y*EA | S | O | O | O | O | S | OMe | OMe |
| 3663a-l | Y*EA | S | O | O | O | O | S | H | OMe |
| 3664a-l | Y*EA | S | O | O | O | O | S | F | OMe |
| 3665a-l | Y*EA | S | O | O | O | O | S | OEt | OMe |
| 3666a-l | Y*EA | S | S | O | O | O | S | OH | OMe |
| 3667a-l | Y*EA | S | S | O | O | O | S | OMe | OMe |
| 3668a-l | Y*EA | S | S | O | O | O | S | H | OMe |
| 3669a-l | Y*EA | S | S | O | O | O | S | F | OMe |
| 3670a-l | Y*EA | S | S | O | O | O | S | OEt | OMe |
| 3671a-l | Y*EA | O | O | S | O | S | O | OH | OMe |
| 3672a-l | Y*EA | O | O | S | O | S | O | OMe | OMe |
| 3673a-l | Y*EA | O | O | S | O | S | O | H | OMe |
| 3674a-l | Y*EA | O | O | S | O | S | O | F | OMe |
| 3675a-l | Y*EA | O | O | S | O | S | O | OEt | OMe |
| 3676a-l | Y*EA | S | O | S | O | S | O | OH | OMe |
| 3677a-l | Y*EA | S | O | S | O | S | O | OMe | OMe |
| 3678a-l | Y*EA | S | O | S | O | S | O | H | OMe |
| 3679a-l | Y*EA | S | O | S | O | S | O | F | OMe |
| 3680a-l | Y*EA | S | O | S | O | S | O | OEt | OMe |
| 3681a-l | Y*EA | S | S | S | O | S | O | OH | OMe |
| 3682a-l | Y*EA | S | S | S | O | S | O | OMe | OMe |
| 3683a-l | Y*EA | S | S | S | O | S | O | H | OMe |
| 3684a-l | Y*EA | S | S | S | O | S | O | F | OMe |
| 3685a-l | Y*EA | S | S | S | O | S | O | OEt | OMe |
| 3686a-l | Y*EA | O | O | O | S | O | S | OH | OMe |
| 3687a-l | Y*EA | O | O | O | S | O | S | OMe | OMe |
| 3688a-l | Y*EA | O | O | O | S | O | S | H | OMe |
| 3689a-l | Y*EA | O | O | O | S | O | S | F | OMe |
| 3690a-l | Y*EA | O | O | O | S | O | S | OEt | OMe |
| 3691a-l | Y*EA | S | O | O | S | O | S | OH | OMe |
| 3692a-l | Y*EA | S | O | O | S | O | S | OMe | OMe |
| 3693a-l | Y*EA | S | O | O | S | O | S | H | OMe |
| 3694a-l | Y*EA | S | O | O | S | O | S | F | OMe |
| 3695a-l | Y*EA | S | O | O | S | O | S | OEt | OMe |
| 3696a-l | Y*EA | S | S | O | S | O | S | OH | OMe |
| 3697a-l | Y*EA | S | S | O | S | O | S | OMe | OMe |
| 3698a-l | Y*EA | S | S | O | S | O | S | H | OMe |
| 3699a-l | Y*EA | S | S | O | S | O | S | F | OMe |
| 3700a-l | Y*EA | S | S | O | S | O | S | OEt | OMe |
| 3701a-l | Y*EC | O | O | O | O | O | O | OH | OH |
| 3702a-l | Y*EC | O | O | O | O | O | O | OMe | OH |
| 3703a-l | Y*EC | O | O | O | O | O | O | H | OH |
| 3704a-l | Y*EC | O | O | O | O | O | O | F | OH |
| 3705a-l | Y*EC | O | O | O | O | O | O | OEt | OH |
| 3706a-l | Y*EC | S | O | O | O | O | O | OH | OH |
| 3707a-l | Y*EC | S | O | O | O | O | O | OMe | OH |
| 3708a-l | Y*EC | S | O | O | O | O | O | H | OH |
| 3709a-l | Y*EC | S | O | O | O | O | O | F | OH |
| 3710a-l | Y*EC | S | O | O | O | O | O | OEt | OH |
| 3711a-l | Y*EC | S | S | O | O | O | O | OH | OH |
| 3712a-l | Y*EC | S | S | O | O | O | O | OMe | OH |
| 3713a-l | Y*EC | S | S | O | O | O | O | H | OH |
| 3714a-l | Y*EC | S | S | O | O | O | O | F | OH |
| 3715a-l | Y*EC | S | S | O | O | O | O | OEt | OH |
| 3716a-l | Y*EC | O | O | S | O | O | O | OH | OH |
| 3717a-l | Y*EC | O | O | S | O | O | O | OMe | OH |
| 3718a-l | Y*EC | O | O | S | O | O | O | H | OH |
| 3719a-l | Y*EC | O | O | S | O | O | O | F | OH |
| 3720a-l | Y*EC | O | O | S | O | O | O | OEt | OH |
| 3721a-l | Y*EC | S | O | S | O | O | O | OH | OH |
| 3722a-l | Y*EC | S | O | S | O | O | O | OMe | OH |
| 3723a-l | Y*EC | S | O | S | O | O | O | H | OH |
| 3724a-l | Y*EC | S | O | S | O | O | O | F | OH |
| 3725a-l | Y*EC | S | O | S | O | O | O | OEt | OH |
| 3726a-l | Y*EC | S | S | S | O | O | O | OH | OH |
| 3727a-l | Y*EC | S | S | S | O | O | O | OMe | OH |
| 3728a-l | Y*EC | S | S | S | O | O | O | H | OH |
| 3729a-l | Y*EC | S | S | S | O | O | O | F | OH |
| 3730a-l | Y*EC | S | S | S | O | O | O | OEt | OH |
| 3731a-l | Y*EC | O | O | S | S | O | O | OH | OH |
| 3732a-l | Y*EC | O | O | S | S | O | O | OMe | OH |
| 3733a-l | Y*EC | O | O | S | S | O | O | H | OH |
| 3734a-l | Y*EC | O | O | S | S | O | O | F | OH |
| 3735a-l | Y*EC | O | O | S | S | O | O | OEt | OH |
| 3736a-l | Y*EC | S | O | S | S | O | O | OH | OH |
| 3737a-l | Y*EC | S | O | S | S | O | O | OMe | OH |
| 3738a-l | Y*EC | S | O | S | S | O | O | H | OH |
| 3739a-l | Y*EC | S | O | S | S | O | O | F | OH |
| 3740a-l | Y*EC | S | O | S | S | O | O | OEt | OH |
| 3741a-l | Y*EC | S | S | S | S | O | O | OH | OH |
| 3742a-l | Y*EC | S | S | S | S | O | O | OMe | OH |
| 3743a-l | Y*EC | S | S | S | S | O | O | H | OH |
| 3744a-l | Y*EC | S | S | S | S | O | O | F | OH |
| 3745a-l | Y*EC | S | S | S | S | O | O | OEt | OH |
| 3746a-l | Y*EC | O | O | S | S | S | O | OH | OH |
| 3747a-l | Y*EC | O | O | S | S | S | O | OMe | OH |
| 3748a-l | Y*EC | O | O | S | S | S | O | H | OH |
| 3749a-l | Y*EC | O | O | S | S | S | O | F | OH |
| 3750a-l | Y*EC | O | O | S | S | S | O | OEt | OH |
| 3751a-l | Y*EC | S | O | S | S | S | O | OH | OH |
| 3752a-l | Y*EC | S | O | S | S | S | O | OMe | OH |
| 3753a-l | Y*EC | S | O | S | S | S | O | H | OH |
| 3754a-l | Y*EC | S | O | S | S | S | O | F | OH |
| 3755a-l | Y*EC | S | O | S | S | S | O | OEt | OH |
| 3756a-l | Y*EC | S | S | S | S | S | O | OH | OH |
| 3757a-l | Y*EC | S | S | S | S | S | O | OMe | OH |
| 3758a-l | Y*EC | S | S | S | S | S | O | H | OH |
| 3759a-l | Y*EC | S | S | S | S | S | O | F | OH |
| 3760a-l | Y*EC | S | S | S | S | S | O | OEt | OH |
| 3761a-l | Y*EC | O | O | S | S | S | S | OH | OH |
| 3762a-l | Y*EC | O | O | S | S | S | S | OMe | OH |
| 3763a-l | Y*EC | O | O | S | S | S | S | H | OH |
| 3764a-l | Y*EC | O | O | S | S | S | S | F | OH |
| 3765a-l | Y*EC | O | O | S | S | S | S | OEt | OH |
| 3766a-l | Y*EC | S | O | S | S | S | S | OH | OH |
| 3767a-l | Y*EC | S | O | S | S | S | S | OMe | OH |
| 3768a-l | Y*EC | S | O | S | S | S | S | H | OH |
| 3769a-l | Y*EC | S | O | S | S | S | S | F | OH |
| 3770a-l | Y*EC | S | O | S | S | S | S | OEt | OH |
| 3771a-l | Y*EC | S | S | S | S | S | S | OF | OH |
| 3772a-l | Y*EC | S | S | S | S | S | S | OMe | OH |
| 3773a-l | Y*EC | S | S | S | S | S | S | H | OH |
| 3774a-l | Y*EC | S | S | S | S | S | S | F | OH |
| 3775a-l | Y*EC | S | S | S | S | S | S | OEt | OH |
| 3776a-l | Y*EC | O | O | O | S | S | S | OH | OH |
| 3777a-l | Y*EC | O | O | O | S | S | S | OMe | OH |
| 3778a-l | Y*EC | O | O | O | S | S | S | H | OH |
| 3779a-l | Y*EC | O | O | O | S | S | S | F | OH |
| 3780a-l | Y*EC | O | O | O | S | S | S | OEt | OH |
| 3781a-l | Y*EC | S | O | O | S | S | S | OH | OH |
| 3782a-l | Y*EC | S | O | O | S | S | S | OMe | OH |
| 3783a-l | Y*EC | S | O | O | S | S | S | H | OH |
| 3784a-l | Y*EC | S | O | O | S | S | S | F | OH |
| 3785a-l | Y*EC | S | O | O | S | S | S | OEt | OH |
| 3786a-l | Y*EC | S | S | O | S | S | S | OH | OH |
| 3787a-l | Y*EC | S | S | O | S | S | S | OMe | OH |
| 3788a-l | Y*EC | S | S | O | S | S | S | H | OH |
| 3789a-l | Y*EC | S | S | O | S | S | S | F | OH |
| 3790a-l | Y*EC | S | S | O | S | S | S | OEt | OH |
| 3791a-l | Y*EC | O | O | O | O | S | S | OH | OH |
| 3792a-l | Y*EC | O | O | O | O | S | S | OMe | OH |
| 3793a-l | Y*EC | O | O | O | O | S | S | H | OH |
| 3794a-l | Y*EC | O | O | O | O | S | S | F | OH |
| 3795a-l | Y*EC | O | O | O | O | S | S | OEt | OH |
| 3796a-l | Y*EC | S | O | O | O | S | S | OH | OH |
| 3797a-l | Y*EC | S | O | O | O | S | S | OMe | OH |
| 3798a-l | Y*EC | S | O | O | O | S | S | H | OH |
| 3799a-l | Y*EC | S | O | O | O | S | S | F | OH |
| 3800a-l | Y*EC | S | O | O | O | S | S | OEt | OH |
| 3801a-l | Y*EC | S | S | O | O | S | S | OH | OH |
| 3802a-l | Y*EC | S | S | O | O | S | S | OMe | OH |
| 3803a-l | Y*EC | S | S | O | O | S | S | H | OH |
| 3804a-l | Y*EC | S | S | O | O | S | S | F | OH |
| 3805a-l | Y*EC | S | S | O | O | S | S | OEt | OH |
| 3806a-l | Y*EC | O | O | O | O | O | S | OH | OH |
| 3807a-l | Y*EC | O | O | O | O | O | S | OMe | OH |
| 3808a-l | Y*EC | O | O | O | O | O | S | H | OH |
| 3809a-l | Y*EC | O | O | O | O | O | S | F | OH |
| 3810a-l | Y*EC | O | O | O | O | O | S | OEt | OH |
| 3811a-l | Y*EC | S | O | O | O | O | S | OH | OH |
| 3812a-l | Y*EC | S | O | O | O | O | S | OMe | OH |
| 3813a-l | Y*EC | S | O | O | O | O | S | H | OH |
| 3814a-l | Y*EC | S | O | O | O | O | S | F | OH |
| 3815a-l | Y*EC | S | O | O | O | O | S | OEt | OH |
| 3816a-l | Y*EC | S | S | O | O | O | S | OH | OH |
| 3817a-l | Y*EC | S | S | O | O | O | S | OMe | OH |
| 3818a-l | Y*EC | S | S | O | O | O | S | H | OH |
| 3819a-l | Y*EC | S | S | O | O | O | S | F | OH |
| 3820a-l | Y*EC | S | S | O | O | O | S | OEt | OH |
| 3821a-l | Y*EC | O | O | S | O | S | O | OH | OH |
| 3822a-l | Y*EC | O | O | S | O | S | O | OMe | OH |
| 3823a-l | Y*EC | O | O | S | O | S | O | H | OH |
| 3824a-l | Y*EC | O | O | S | O | S | O | F | OH |
| 3825a-l | Y*EC | O | O | S | O | S | O | OEt | OH |
| 3826a-l | Y*EC | S | O | S | O | S | O | OH | OH |
| 3827a-l | Y*EC | S | O | S | O | S | O | OMe | OH |
| 3828a-l | Y*EC | S | O | S | O | S | O | H | OH |
| 3829a-l | Y*EC | S | O | S | O | S | O | F | OH |
| 3830a-l | Y*EC | S | O | S | O | S | O | OEt | OH |
| 3831a-l | Y*EC | S | S | S | O | S | O | OH | OH |
| 3832a-l | Y*EC | S | S | S | O | S | O | OMe | OH |
| 3833a-l | Y*EC | S | S | S | O | S | O | H | OH |
| 3834a-l | Y*EC | S | S | S | O | S | O | F | OH |
| 3835a-l | Y*EC | S | S | S | O | S | O | OEt | OH |
| 3836a-l | Y*EC | O | O | O | S | O | S | OH | OH |
| 3837a-l | Y*EC | O | O | O | S | O | S | OMe | OH |
| 3838a-l | Y*EC | O | O | O | S | O | S | H | OH |
| 3839a-l | Y*EC | O | O | O | S | O | S | F | OH |
| 3840a-l | Y*EC | O | O | O | S | O | S | OEt | OH |
| 3841a-l | Y*EC | S | O | O | S | O | S | OH | OH |
| 3842a-l | Y*EC | S | O | O | S | O | S | OMe | OH |
| 3843a-l | Y*EC | S | O | O | S | O | S | H | OH |
| 3844a-l | Y*EC | S | O | O | S | O | S | F | OH |
| 3845a-l | Y*EC | S | O | O | S | O | S | OEt | OH |
| 3846a-l | Y*EC | S | S | O | S | O | S | OH | OH |
| 3847a-l | Y*EC | S | S | O | S | O | S | OMe | OH |
| 3848a-l | Y*EC | S | S | O | S | O | S | H | OH |
| 3849a-l | Y*EC | S | S | O | S | O | S | F | OH |
| 3850a-l | Y*EC | S | S | O | S | O | S | OEt | OH |
| 3851a-l | Y*EC | O | O | O | O | O | O | OH | OMe |
| 3852a-l | Y*EC | O | O | O | O | O | O | OMe | OMe |
| 3853a-l | Y*EC | O | O | O | O | O | O | H | OMe |
| 3854a-l | Y*EC | O | O | O | O | O | O | F | OMe |
| 3855a-l | Y*EC | O | O | O | O | O | O | OEt | OMe |
| 3856a-l | Y*EC | S | O | O | O | O | O | OH | OMe |
| 3857a-l | Y*EC | S | O | O | O | O | O | OMe | OMe |
| 3858a-l | Y*EC | S | O | O | O | O | O | H | OMe |
| 3859a-l | Y*EC | S | O | O | O | O | O | F | OMe |
| 3860a-l | Y*EC | S | O | O | O | O | O | OEt | OMe |
| 3861a-l | Y*EC | S | S | O | O | O | O | OH | OMe |
| 3862a-l | Y*EC | S | S | O | O | O | O | OMe | OMe |
| 3863a-l | Y*EC | S | S | O | O | O | O | H | OMe |
| 3864a-l | Y*EC | S | S | O | O | O | O | F | OMe |
| 3865a-l | Y*EC | S | S | O | O | O | O | OEt | OMe |
| 3866a-l | Y*EC | O | O | S | O | O | O | OH | OMe |
| 3867a-l | Y*EC | O | O | S | O | O | O | OMe | OMe |
| 3868a-l | Y*EC | O | O | S | O | O | O | H | OMe |
| 3869a-l | Y*EC | O | O | S | O | O | O | F | OMe |
| 3870a-l | Y*EC | O | O | S | O | O | O | OEt | OMe |
| 3871a-l | Y*EC | S | O | S | O | O | O | OH | OMe |
| 3872a-l | Y*EC | S | O | S | O | O | O | OMe | OMe |
| 3873a-l | Y*EC | S | O | S | O | O | O | H | OMe |
| 3874a-l | Y*EC | S | O | S | O | O | O | F | OMe |
| 3875a-l | Y*EC | S | O | S | O | O | O | OEt | OMe |
| 3876a-l | Y*EC | S | S | S | O | O | O | OH | OMe |
| 3877a-l | Y*EC | S | S | S | O | O | O | OMe | OMe |
| 3878a-l | Y*EC | S | S | S | O | O | O | H | OMe |
| 3879a-l | Y*EC | S | S | S | O | O | O | F | OMe |
| 3880a-l | Y*EC | S | S | S | O | O | O | OEt | OMe |
| 3881a-l | Y*EC | O | O | S | S | O | O | OH | OMe |
| 3882a-l | Y*EC | O | O | S | S | O | O | OMe | OMe |
| 3883a-l | Y*EC | O | O | S | S | O | O | H | OMe |
| 3884a-l | Y*EC | O | O | S | S | O | O | F | OMe |
| 3885a-l | Y*EC | O | O | S | S | O | O | OEt | OMe |
| 3886a-l | Y*EC | S | O | S | S | O | O | OH | OMe |
| 3887a-l | Y*EC | S | O | S | S | O | O | OMe | OMe |
| 3888a-l | Y*EC | S | O | S | S | O | O | H | OMe |
| 3889a-l | Y*EC | S | O | S | S | O | O | F | OMe |
| 3890a-l | Y*EC | S | O | S | S | O | O | OEt | OMe |
| 3891a-l | Y*EC | S | S | S | S | O | O | OH | OMe |
| 3892a-l | Y*EC | S | S | S | S | O | O | OMe | OMe |
| 3893a-l | Y*EC | S | S | S | S | O | O | H | OMe |
| 3894a-l | Y*EC | S | S | S | S | O | O | F | OMe |
| 3895a-l | Y*EC | S | S | S | S | O | O | OEt | OMe |
| 3896a-l | Y*EC | O | O | S | S | S | O | OH | OMe |
| 3897a-l | Y*EC | O | O | S | S | S | O | OMe | OMe |
| 3898a-l | Y*EC | O | O | S | S | S | O | H | OMe |
| 3899a-l | Y*EC | O | O | S | S | S | O | F | OMe |
| 3900a-l | Y*EC | O | O | S | S | S | O | OEt | OMe |
| 3901a-l | Y*EC | S | O | S | S | S | O | OH | OMe |
| 3902a-l | Y*EC | S | O | S | S | S | O | OMe | OMe |
| 3903a-l | Y*EC | S | O | S | S | S | O | H | OMe |
| 3904a-l | Y*EC | S | O | S | S | S | O | F | OMe |
| 3905a-l | Y*EC | S | O | S | S | S | O | OEt | OMe |
| 3906a-l | Y*EC | S | S | S | S | S | O | OH | OMe |
| 3907a-l | Y*EC | S | S | S | S | S | O | OMe | OMe |
| 3908a-l | Y*EC | S | S | S | S | S | O | H | OMe |
| 3909a-l | Y*EC | S | S | S | S | S | O | F | OMe |
| 3910a-l | Y*EC | S | S | S | S | S | O | OEt | OMe |
| 3911a-l | Y*EC | O | O | S | S | S | S | OH | OMe |
| 3912a-l | Y*EC | O | O | S | S | S | S | OMe | OMe |
| 3913a-l | Y*EC | O | O | S | S | S | S | H | OMe |
| 3914a-l | Y*EC | O | O | S | S | S | S | F | OMe |
| 3915a-l | Y*EC | O | O | S | S | S | S | OEt | OMe |
| 3916a-l | Y*EC | S | O | S | S | S | S | OH | OMe |
| 3917a-l | Y*EC | S | O | S | S | S | S | OMe | OMe |
| 3918a-l | Y*EC | S | O | S | S | S | S | H | OMe |
| 3919a-l | Y*EC | S | O | S | S | S | S | F | OMe |
| 3920a-l | Y*EC | S | O | S | S | S | S | OEt | OMe |
| 3921a-l | Y*EC | S | S | S | S | S | S | OH | OMe |
| 3922a-l | Y*EC | S | S | S | S | S | S | OMe | OMe |
| 3923a-l | Y*EC | S | S | S | S | S | S | H | OMe |
| 3924a-l | Y*EC | S | S | S | S | S | S | F | OMe |
| 3925a-l | Y*EC | S | S | S | S | S | S | OEt | OMe |
| 3926a-l | Y*EC | O | O | O | S | S | S | OH | OMe |
| 3927a-l | Y*EC | O | O | O | S | S | S | OMe | OMe |
| 3928a-l | Y*EC | O | O | O | S | S | S | H | OMe |
| 3929a-l | Y*EC | O | O | O | S | S | S | F | OMe |
| 3930a-l | Y*EC | O | O | O | S | S | S | OEt | OMe |
| 3931a-l | Y*EC | S | O | O | S | S | S | OH | OMe |
| 3932a-l | Y*EC | S | O | O | S | S | S | OMe | OMe |
| 3933a-l | Y*EC | S | O | O | S | S | S | H | OMe |
| 3934a-l | Y*EC | S | O | O | S | S | S | F | OMe |
| 3935a-l | Y*EC | S | O | O | S | S | S | OEt | OMe |
| 3936a-l | Y*EC | S | S | O | S | S | S | OH | OMe |
| 3937a-l | Y*EC | S | S | O | S | S | S | OMe | OMe |
| 3938a-l | Y*EC | S | S | O | S | S | S | H | OMe |
| 3939a-l | Y*EC | S | S | O | S | S | S | F | OMe |
| 3940a-l | Y*EC | S | S | O | S | S | S | OEt | OMe |
| 3941a-l | Y*EC | O | O | O | O | S | S | OH | OMe |
| 3942a-l | Y*EC | O | O | O | O | S | S | OMe | OMe |
| 3943a-l | Y*EC | O | O | O | O | S | S | H | OMe |
| 3944a-l | Y*EC | O | O | O | O | S | S | F | OMe |
| 3945a-l | Y*EC | O | O | O | O | S | S | OEt | OMe |
| 3946a-l | Y*EC | S | O | O | O | S | S | OH | OMe |
| 3947a-l | Y*EC | S | O | O | O | S | S | OMe | OMe |
| 3948a-l | Y*EC | S | O | O | O | S | S | H | OMe |
| 3949a-l | Y*EC | S | O | O | O | S | S | F | OMe |
| 3950a-l | Y*EC | S | O | O | O | S | S | OEt | OMe |
| 3951a-l | Y*EC | S | S | O | O | S | S | OH | OMe |
| 3952a-l | Y*EC | S | S | O | O | S | S | OMe | OMe |
| 3953a-l | Y*EC | S | S | O | O | S | S | H | OMe |
| 3954a-l | Y*EC | S | S | O | O | S | S | F | OMe |
| 3955a-l | Y*EC | S | S | O | O | S | S | OEt | OMe |
| 3956a-l | Y*EC | O | O | O | O | O | S | OH | OMe |
| 3957a-l | Y*EC | O | O | O | O | O | S | OMe | OMe |
| 3958a-l | Y*EC | O | O | O | O | O | S | H | OMe |
| 3959a-l | Y*EC | O | O | O | O | O | S | F | OMe |
| 3960a-l | Y*EC | O | O | O | O | O | S | OEt | OMe |
| 3961a-l | Y*EC | S | O | O | O | O | S | OH | OMe |
| 3962a-l | Y*EC | S | O | O | O | O | S | OMe | OMe |
| 3963a-l | Y*EC | S | O | O | O | O | S | H | OMe |
| 3964a-l | Y*EC | S | O | O | O | O | S | F | OMe |
| 3965a-l | Y*EC | S | O | O | O | O | S | OEt | OMe |
| 3966a-l | Y*EC | S | S | O | O | O | S | OH | OMe |
| 3967a-l | Y*EC | S | S | O | O | O | S | OMe | OMe |
| 3968a-l | Y*EC | S | S | O | O | O | S | H | OMe |
| 3969a-l | Y*EC | S | S | O | O | O | S | F | OMe |
| 3970a-l | Y*EC | S | S | O | O | O | S | OEt | OMe |
| 3971a-l | Y*EC | O | O | S | O | S | O | OH | OMe |
| 3972a-l | Y*EC | O | O | S | O | S | O | OMe | OMe |
| 3973a-l | Y*EC | O | O | S | O | S | O | H | OMe |
| 3974a-l | Y*EC | O | O | S | O | S | O | F | OMe |
| 3975a-l | Y*EC | O | O | S | O | S | O | OEt | OMe |
| 3976a-l | Y*EC | S | O | S | O | S | O | OH | OMe |
| 3977a-l | Y*EC | S | O | S | O | S | O | OMe | OMe |
| 3978a-l | Y*EC | S | O | S | O | S | O | H | OMe |
| 3979a-l | Y*EC | S | O | S | O | S | O | F | OMe |
| 3980a-l | Y*EC | S | O | S | O | S | O | OEt | OMe |
| 3981a-l | Y*EC | S | S | S | O | S | O | OH | OMe |
| 3982a-l | Y*EC | S | S | S | O | S | O | OMe | OMe |
| 3983a-l | Y*EC | S | S | S | O | S | O | H | OMe |
| 3984a-l | Y*EC | S | S | S | O | S | O | F | OMe |
| 3985a-l | Y*EC | S | S | S | O | S | O | OEt | OMe |
| 3986a-l | Y*EC | O | O | O | S | O | S | OH | OMe |
| 3987a-l | Y*EC | O | O | O | S | O | S | OMe | OMe |
| 3988a-l | Y*EC | O | O | O | S | O | S | H | OMe |
| 3989a-l | Y*EC | O | O | O | S | O | S | F | OMe |
| 3990a-l | Y*EC | O | O | O | S | O | S | OEt | OMe |
| 3991a-l | Y*EC | S | O | O | S | O | S | OH | OMe |
| 3992a-l | Y*EC | S | O | O | S | O | S | OMe | OMe |
| 3993a-l | Y*EC | S | O | O | S | O | S | H | OMe |
| 3994a-l | Y*EC | S | O | O | S | O | S | F | OMe |
| 3995a-l | Y*EC | S | O | O | S | O | S | OEt | OMe |
| 3996a-l | Y*EC | S | S | O | S | O | S | OH | OMe |
| 3997a-l | Y*EC | S | S | O | S | O | S | OMe | OMe |
| 3998a-l | Y*EC | S | S | O | S | O | S | H | OMe |
| 3999a-l | Y*EC | S | S | O | S | O | S | F | OMe |
| 4000a-l | Y*EC | S | S | O | S | O | S | OEt | OMe |
| 4001a-l | Y*EU | O | O | O | O | O | O | OH | OH |
| 4002a-l | Y*EU | O | O | O | O | O | O | OMe | OH |
| 4003a-l | Y*EU | O | O | O | O | O | O | H | OH |
| 4004a-l | Y*EU | O | O | O | O | O | O | F | OH |
| 4005a-l | Y*EU | O | O | O | O | O | O | OEt | OH |
| 4006a-l | Y*EU | S | O | O | O | O | O | OH | OH |
| 4007a-l | Y*EU | S | O | O | O | O | O | OMe | OH |
| 4008a-l | Y*EU | S | O | O | O | O | O | H | OH |
| 4009a-l | Y*EU | S | O | O | O | O | O | F | OH |
| 4010a-l | Y*EU | S | O | O | O | O | O | OEt | OH |
| 4011a-l | Y*EU | S | S | O | O | O | O | OH | OH |
| 4012a-l | Y*EU | S | S | O | O | O | O | OMe | OH |
| 4013a-l | Y*EU | S | S | O | O | O | O | H | OH |
| 4014a-l | Y*EU | S | S | O | O | O | O | F | OH |
| 4015a-l | Y*EU | S | S | O | O | O | O | OEt | OH |
| 4016a-l | Y*EU | O | O | S | O | O | O | OH | OH |
| 4017a-l | Y*EU | O | O | S | O | O | O | OMe | OH |
| 4018a-l | Y*EU | O | O | S | O | O | O | H | OH |
| 4019a-l | Y*EU | O | O | S | O | O | O | F | OH |
| 4020a-l | Y*EU | O | O | S | O | O | O | OEt | OH |
| 4021a-l | Y*EU | S | O | S | O | O | O | OH | OH |
| 4022a-l | Y*EU | S | O | S | O | O | O | OMe | OH |
| 4023a-l | Y*EU | S | O | S | O | O | O | H | OH |
| 4024a-l | Y*EU | S | O | S | O | O | O | F | OH |
| 4025a-l | Y*EU | S | O | S | O | O | O | OEt | OH |
| 4026a-l | Y*EU | S | S | S | O | O | O | OH | OH |
| 4027a-l | Y*EU | S | S | S | O | O | O | OMe | OH |
| 4028a-l | Y*EU | S | S | S | O | O | O | H | OH |
| 4029a-l | Y*EU | S | S | S | O | O | O | F | OH |
| 4030a-l | Y*EU | S | S | S | O | O | O | OEt | OH |
| 4031a-l | Y*EU | O | O | S | S | O | O | OH | OH |
| 4032a-l | Y*EU | O | O | S | S | O | O | OMe | OH |
| 4033a-l | Y*EU | O | O | S | S | O | O | H | OH |
| 4034a-l | Y*EU | O | O | S | S | O | O | F | OH |
| 4035a-l | Y*EU | O | O | S | S | O | O | OEt | OH |
| 4036a-l | Y*EU | S | O | S | S | O | O | OH | OH |
| 4037a-l | Y*EU | S | O | S | S | O | O | OMe | OH |
| 4038a-l | Y*EU | S | O | S | S | O | O | H | OH |
| 4039a-l | Y*EU | S | O | S | S | O | O | F | OH |
| 4040a-l | Y*EU | S | O | S | S | O | O | OEt | OH |
| 4041a-l | Y*EU | S | S | S | S | O | O | OH | OH |
| 4042a-l | Y*EU | S | S | S | S | O | O | OMe | OH |
| 4043a-l | Y*EU | S | S | S | S | O | O | H | OH |
| 4044a-l | Y*EU | S | S | S | S | O | O | F | OH |
| 4045a-l | Y*EU | S | S | S | S | O | O | OEt | OH |
| 4046a-l | Y*EU | O | O | S | S | S | O | OH | OH |
| 4047a-l | Y*EU | O | O | S | S | S | O | OMe | OH |
| 4048a-l | Y*EU | O | O | S | S | S | O | H | OH |
| 4049a-l | Y*EU | O | O | S | S | S | O | F | OH |
| 4050a-l | Y*EU | O | O | S | S | S | O | OEt | OH |
| 4051a-l | Y*EU | S | O | S | S | S | O | OH | OH |
| 4052a-l | Y*EU | S | O | S | S | S | O | OMe | OH |
| 4053a-l | Y*EU | S | O | S | S | S | O | H | OH |
| 4054a-l | Y*EU | S | O | S | S | S | O | F | OH |
| 4055a-l | Y*EU | S | O | S | S | S | O | OEt | OH |
| 4056a-l | Y*EU | S | S | S | S | S | O | OH | OH |
| 4057a-l | Y*EU | S | S | S | S | S | O | OMe | OH |
| 4058a-l | Y*EU | S | S | S | S | S | O | H | OH |
| 4059a-l | Y*EU | S | S | S | S | S | O | F | OH |
| 4060a-l | Y*EU | S | S | S | S | S | O | OEt | OH |
| 4061a-l | Y*EU | O | O | S | S | S | S | OH | OH |
| 4062a-l | Y*EU | O | O | S | S | S | S | OMe | OH |
| 4063a-l | Y*EU | O | O | S | S | S | S | H | OH |
| 4064a-l | Y*EU | O | O | S | S | S | S | F | OH |
| 4065a-l | Y*EU | O | O | S | S | S | S | OEt | OH |
| 4066a-l | Y*EU | S | O | S | S | S | S | OH | OH |
| 4067a-l | Y*EU | S | O | S | S | S | S | OMe | OH |
| 4068a-l | Y*EU | S | O | S | S | S | S | H | OH |
| 4069a-l | Y*EU | S | O | S | S | S | S | F | OH |
| 4070a-l | Y*EU | S | O | S | S | S | S | OEt | OH |
| 4071a-l | Y*EU | S | S | S | S | S | S | OH | OH |
| 4072a-l | Y*EU | S | S | S | S | S | S | OMe | OH |
| 4073a-l | Y*EU | S | S | S | S | S | S | H | OH |
| 4074a-l | Y*EU | S | S | S | S | S | S | F | OH |
| 4075a-l | Y*EU | S | S | S | S | S | S | OEt | OH |
| 4076a-l | Y*EU | O | O | O | S | S | S | OH | OH |
| 4077a-l | Y*EU | O | O | O | S | S | S | OMe | OH |
| 4078a-l | Y*EU | O | O | O | S | S | S | H | OH |
| 4079a-l | Y*EU | O | O | O | S | S | S | F | OH |
| 4080a-l | Y*EU | O | O | O | S | S | S | OEt | OH |
| 4081a-l | Y*EU | S | O | O | S | S | S | OH | OH |
| 4082a-l | Y*EU | S | O | O | S | S | S | OMe | OH |
| 4083a-l | Y*EU | S | O | O | S | S | S | H | OH |
| 4084a-l | Y*EU | S | O | O | S | S | S | F | OH |
| 4085a-l | Y*EU | S | O | O | S | S | S | OEt | OH |
| 4086a-l | Y*EU | S | S | O | S | S | S | OH | OH |
| 4087a-l | Y*EU | S | S | O | S | S | S | OMe | OH |
| 4088a-l | Y*EU | S | S | O | S | S | S | H | OH |
| 4089a-l | Y*EU | S | S | O | S | S | S | F | OH |
| 4090a-l | Y*EU | S | S | O | S | S | S | OEt | OH |
| 4091a-l | Y*EU | O | O | O | O | S | S | OH | OH |
| 4092a-l | Y*EU | O | O | O | O | S | S | OMe | OH |
| 4093a-l | Y*EU | O | O | O | O | S | S | H | OH |
| 4094a-l | Y*EU | O | O | O | O | S | S | F | OH |
| 4095a-l | Y*EU | O | O | O | O | S | S | OEt | OH |
| 4096a-l | Y*EU | S | O | O | O | S | S | OH | OH |
| 4097a-l | Y*EU | S | O | O | O | S | S | OMe | OH |
| 4098a-l | Y*EU | S | O | O | O | S | S | H | OH |
| 4099a-l | Y*EU | S | O | O | O | S | S | F | OH |
| 4100a-l | Y*EU | S | O | O | O | S | S | OEt | OH |
| 4101a-l | Y*EU | S | S | O | O | S | S | OH | OH |
| 4102a-l | Y*EU | S | S | O | O | S | S | OMe | OH |
| 4103a-l | Y*EU | S | S | O | O | S | S | H | OH |
| 4104a-l | Y*EU | S | S | O | O | S | S | F | OH |
| 4105a-l | Y*EU | S | S | O | O | S | S | OEt | OH |
| 4106a-l | Y*EU | O | O | O | O | O | S | OH | OH |
| 4107a-l | Y*EU | O | O | O | O | O | S | OMe | OH |
| 4108a-l | Y*EU | O | O | O | O | O | S | H | OH |
| 4109a-l | Y*EU | O | O | O | O | O | S | F | OF |
| 4110a-l | Y*EU | O | O | O | O | O | S | OEt | OH |
| 4111a-l | Y*EU | S | O | O | O | O | S | OH | OH |
| 4112a-l | Y*EU | S | O | O | O | O | S | OMe | OH |
| 4113a-l | Y*EU | S | O | O | O | O | S | H | OH |
| 4114a-l | Y*EU | S | O | O | O | O | S | F | OH |
| 4115a-l | Y*EU | S | O | O | O | O | S | OEt | OH |
| 4116a-l | Y*EU | S | S | O | O | O | S | OH | OH |
| 4117a-l | Y*EU | S | S | O | O | O | S | OMe | OH |
| 4118a-l | Y*EU | S | S | O | O | O | S | H | OH |
| 4119a-l | Y*EU | S | S | O | O | O | S | F | OH |
| 4120a-l | Y*EU | S | S | O | O | O | S | OEt | OH |
| 4121a-l | Y*EU | O | O | S | O | S | O | OH | OH |
| 4122a-l | Y*EU | O | O | S | O | S | O | OMe | OH |
| 4123a-l | Y*EU | O | O | S | O | S | O | H | OH |
| 4124a-l | Y*EU | O | O | S | O | S | O | F | OH |
| 4125a-l | Y*EU | O | O | S | O | S | O | OEt | OH |
| 4126a-l | Y*EU | S | O | S | O | S | O | OH | OH |
| 4127a-l | Y*EU | S | O | S | O | S | O | OMe | OH |
| 4128a-l | Y*EU | S | O | S | O | S | C | H | OF |
| 4129a-l | Y*EU | S | O | S | O | S | O | F | OH |
| 4130a-l | Y*EU | S | O | S | O | S | O | OEt | OH |
| 4131a-l | Y*EU | S | S | S | O | S | O | OH | OH |
| 4132a-l | Y*EU | S | S | S | O | S | O | OMe | OH |
| 4133a-l | Y*EU | S | S | S | O | S | O | H | OH |
| 4134a-l | Y*EU | S | S | S | O | S | O | F | OH |
| 4135a-l | Y*EU | S | S | S | O | S | O | OEt | OH |
| 4136a-l | Y*EU | O | O | O | S | O | S | OH | OH |
| 4137a-l | Y*EU | O | O | O | S | O | S | OMe | OH |
| 4138a-l | Y*EU | O | O | O | S | O | S | H | OH |
| 4139a-l | Y*EU | O | O | O | S | O | S | F | OH |
| 4140a-l | Y*EU | O | O | O | S | O | S | OEt | OH |
| 4141a-l | Y*EU | S | O | O | S | O | S | OH | OH |
| 4142a-l | Y*EU | S | O | O | S | O | S | OMe | OH |
| 4143a-l | Y*EU | S | O | O | S | O | S | H | OH |
| 4144a-l | Y*EU | S | O | O | S | O | S | F | OH |
| 4145a-l | Y*EU | S | O | O | S | O | S | OEt | OH |
| 4146a-l | Y*EU | S | S | O | S | O | S | OH | OH |
| 4147a-l | Y*EU | S | S | O | S | O | S | OMe | OH |
| 4148a-l | Y*EU | S | S | O | S | O | S | H | OH |
| 4149a-l | Y*EU | S | S | O | S | O | S | F | OH |
| 4150a-l | Y*EU | S | S | O | S | O | S | OEt | OH |
| 4151a-l | Y*EU | O | O | O | O | O | O | OH | OMe |
| 4152a-l | Y*EU | O | O | O | O | O | O | OMe | OMe |
| 4153a-l | Y*EU | O | O | O | O | O | O | H | OMe |
| 4154a-l | Y*EU | O | O | O | O | O | O | F | OMe |
| 4155a-l | Y*EU | O | O | O | O | O | O | OEt | OMe |
| 4156a-l | Y*EU | S | O | O | O | O | O | OH | OMe |
| 4157a-l | Y*EU | S | O | O | O | O | O | OMe | OMe |
| 4158a-l | Y*EU | S | O | O | O | O | O | H | OMe |
| 4159a-l | Y*EU | S | O | O | O | O | O | F | OMe |
| 4160a-l | Y*EU | S | O | O | O | O | O | OEt | OMe |
| 4161a-l | Y*EU | S | S | O | O | O | O | OH | OMe |
| 4162a-l | Y*EU | S | S | O | O | O | O | OMe | OMe |
| 4163a-l | Y*EU | S | S | O | O | O | O | H | OMe |
| 4164a-l | Y*EU | S | S | O | O | O | O | F | OMe |
| 4165a-l | Y*EU | S | S | O | O | O | O | OEt | OMe |
| 4166a-l | Y*EU | O | O | S | O | O | O | OH | OMe |
| 4167a-l | Y*EU | O | O | S | O | O | O | OMe | OMe |
| 4168a-l | Y*EU | O | O | S | O | O | O | H | OMe |
| 4169a-l | Y*EU | O | O | S | O | O | O | F | OMe |
| 4170a-l | Y*EU | O | O | S | O | O | O | OEt | OMe |
| 4171a-l | Y*EU | S | O | S | O | O | O | OH | OMe |
| 4172a-l | Y*EU | S | O | S | O | O | O | OMe | OMe |
| 4173a-l | Y*EU | S | O | S | O | O | O | H | OMe |
| 4174a-l | Y*EU | S | O | S | O | O | O | F | OMe |
| 4175a-l | Y*EU | S | O | S | O | O | O | OEt | OMe |
| 4176a-l | Y*EU | S | S | S | O | O | O | OH | OMe |
| 4177a-l | Y*EU | S | S | S | O | O | O | OMe | OMe |
| 4178a-l | Y*EU | S | S | S | O | O | O | H | OMe |
| 4179a-l | Y*EU | S | S | S | O | O | O | F | OMe |
| 4180a-l | Y*EU | S | S | S | O | O | O | OEt | OMe |
| 4181a-l | Y*EU | O | O | S | S | O | O | OH | OMe |
| 4182a-l | Y*EU | O | O | S | S | O | O | OMe | OMe |
| 4183a-l | Y*EU | O | O | S | S | O | O | H | OMe |
| 4184a-l | Y*EU | O | O | S | S | O | O | F | OMe |
| 4185a-l | Y*EU | O | O | S | S | O | O | OEt | OMe |
| 4186a-l | Y*EU | S | O | S | S | O | O | OH | OMe |
| 4187a-l | Y*EU | S | O | S | S | O | O | OMe | OMe |
| 4188a-l | Y*EU | S | O | S | S | O | O | H | OMe |
| 4189a-l | Y*EU | S | O | S | S | O | O | F | OMe |
| 4190a-l | Y*EU | S | O | S | S | O | O | OEt | OMe |
| 4191a-l | Y*EU | S | S | S | S | O | O | OH | OMe |
| 4192a-l | Y*EU | S | S | S | S | O | O | OMe | OMe |
| 4193a-l | Y*EU | S | S | S | S | O | O | H | OMe |
| 4194a-l | Y*EU | S | S | S | S | O | O | F | OMe |
| 4195a-l | Y*EU | S | S | S | S | O | O | OEt | OMe |
| 4196a-l | Y*EU | O | O | S | S | S | O | OH | OMe |
| 4197a-l | Y*EU | O | O | S | S | S | O | OMe | OMe |
| 4198a-l | Y*EU | O | O | S | S | S | O | H | OMe |
| 4199a-l | Y*EU | O | O | S | S | S | O | F | OMe |
| 4200a-l | Y*EU | O | O | S | S | S | O | OEt | OMe |
| 4201a-l | Y*EU | S | O | S | S | S | O | OH | OMe |
| 4202a-l | Y*EU | S | O | S | S | S | O | OMe | OMe |
| 4203a-l | Y*EU | S | O | S | S | S | O | H | OMe |
| 4204a-l | Y*EU | S | O | S | S | S | O | F | OMe |
| 4205a-l | Y*EU | S | O | S | S | S | O | OEt | OMe |
| 4206a-l | Y*EU | S | S | S | S | S | O | OH | OMe |
| 4207a-l | Y*EU | S | S | S | S | S | O | OMe | OMe |
| 4208a-l | Y*EU | S | S | S | S | S | O | H | OMe |
| 4209a-l | Y*EU | S | S | S | S | S | O | F | OMe |
| 4210a-l | Y*EU | S | S | S | S | S | O | OEt | OMe |
| 4211a-l | Y*EU | O | O | S | S | S | S | OH | OMe |
| 4212a-l | Y*EU | O | O | S | S | S | S | OMe | OMe |
| 4213a-l | Y*EU | O | O | S | S | S | S | H | OMe |
| 4214a-l | Y*EU | O | O | S | S | S | S | F | OMe |
| 4215a-l | Y*EU | O | O | S | S | S | S | OEt | OMe |
| 4216a-l | Y*EU | S | O | S | S | S | S | OH | OMe |
| 4217a-l | Y*EU | S | O | S | S | S | S | OMe | OMe |
| 4218a-l | Y*EU | S | O | S | S | S | S | H | OMe |
| 4219a-l | Y*EU | S | O | S | S | S | S | F | OMe |
| 4220a-l | Y*EU | S | O | S | S | S | S | OEt | OMe |
| 4221a-l | Y*EU | S | S | S | S | S | S | OH | OMe |
| 4222a-l | Y*EU | S | S | S | S | S | S | OMe | OMe |
| 4223a-l | Y*EU | S | S | S | S | S | S | H | OMe |
| 4224a-l | Y*EU | S | S | S | S | S | S | F | OMe |
| 4225a-l | Y*EU | S | S | S | S | S | S | OEt | OMe |
| 4226a-l | Y*EU | O | O | O | S | S | S | OH | OMe |
| 4227a-l | Y*EU | O | O | O | S | S | S | OMe | OMe |
| 4228a-l | Y*EU | O | O | O | S | S | S | H | OMe |
| 4229a-l | Y*EU | O | O | O | S | S | S | F | OMe |
| 4230a-l | Y*EU | O | O | O | S | S | S | OEt | OMe |
| 4231a-l | Y*EU | S | O | O | S | S | S | OH | OMe |
| 4232a-l | Y*EU | S | O | O | S | S | S | OMe | OMe |
| 4233a-l | Y*EU | S | O | O | S | S | S | H | OMe |
| 4234a-l | Y*EU | S | O | O | S | S | S | F | OMe |
| 4235a-l | Y*EU | S | O | O | S | S | S | OEt | OMe |
| 4236a-l | Y*EU | S | S | O | S | S | S | OH | OMe |
| 4237a-l | Y*EU | S | S | O | S | S | S | OMe | OMe |
| 4238a-l | Y*EU | S | S | O | S | S | S | H | OMe |
| 4239a-l | Y*EU | S | S | O | S | S | S | F | OMe |
| 4240a-l | Y*EU | S | S | O | S | S | S | OEt | OMe |
| 4241a-l | Y*EU | O | O | O | O | S | S | OH | OMe |
| 4242a-l | Y*EU | O | O | O | O | S | S | OMe | OMe |
| 4243a-l | Y*EU | O | O | O | O | S | S | H | OMe |
| 4244a-l | Y*EU | O | O | O | O | S | S | F | OMe |
| 4245a-l | Y*EU | O | O | O | O | S | S | OEt | OMe |
| 4246a-l | Y*EU | S | O | O | O | S | S | OH | OMe |
| 4247a-l | Y*EU | S | O | O | O | S | S | OMe | OMe |
| 4248a-l | Y*EU | S | O | O | O | S | S | H | OMe |
| 4249a-l | Y*EU | S | O | O | O | S | S | F | OMe |
| 4250a-l | Y*EU | S | O | O | O | S | S | OEt | OMe |
| 4251a-l | Y*EU | S | S | O | O | S | S | OH | OMe |
| 4252a-l | Y*EU | S | S | O | O | S | S | OMe | OMe |
| 4253a-l | Y*EU | S | S | O | O | S | S | H | OMe |
| 4254a-l | Y*EU | S | S | O | O | S | S | F | OMe |
| 4255a-l | Y*EU | S | S | O | O | S | S | OEt | OMe |
| 4256a-l | Y*EU | O | O | O | O | O | S | OH | OMe |
| 4257a-l | Y*EU | O | O | O | O | O | S | OMe | OMe |
| 4258a-l | Y*EU | O | O | O | O | O | S | H | OMe |
| 4259a-l | Y*EU | O | O | O | O | O | S | F | OMe |
| 4260a-l | Y*EU | O | O | O | O | O | S | OEt | OMe |
| 4261a-l | Y*EU | S | O | O | O | O | S | OH | OMe |
| 4262a-l | Y*EU | S | O | O | O | O | S | OMe | OMe |
| 4263a-l | Y*EU | S | O | O | O | O | S | H | OMe |
| 4264a-l | Y*EU | S | O | O | O | O | S | F | OMe |
| 4265a-l | Y*EU | S | O | O | O | O | S | OEt | OMe |
| 4266a-l | Y*EU | S | S | O | O | O | S | OH | OMe |
| 4267a-l | Y*EU | S | S | O | O | O | S | OMe | OMe |
| 4268a-l | Y*EU | S | S | O | O | O | S | H | OMe |
| 4269a-l | Y*EU | S | S | O | O | O | S | F | OMe |
| 4270a-l | Y*EU | S | S | O | O | O | S | OEt | OMe |
| 4271a-l | Y*EU | O | O | S | O | S | O | OH | OMe |
| 4272a-l | Y*EU | O | O | S | O | S | O | OMe | OMe |
| 4273a-l | Y*EU | O | O | S | O | S | O | H | OMe |
| 4274a-l | Y*EU | O | O | S | O | S | O | F | OMe |
| 4275a-l | Y*EU | O | O | S | O | S | O | OEt | OMe |
| 4276a-l | Y*EU | S | O | S | O | S | O | OH | OMe |
| 4277a-l | Y*EU | S | O | S | O | S | O | OMe | OMe |
| 4278a-l | Y*EU | S | O | S | O | S | O | H | OMe |
| 4279a-l | Y*EU | S | O | S | O | S | O | F | OMe |
| 4280a-l | Y*EU | S | O | S | O | S | O | OEt | OMe |
| 4281a-l | Y*EU | S | S | S | O | S | O | OH | OMe |
| 4282a-l | Y*EU | S | S | S | O | S | O | OMe | OMe |
| 4283a-l | Y*EU | S | S | S | O | S | O | H | OMe |
| 4284a-l | Y*EU | S | S | S | O | S | O | F | OMe |
| 4285a-l | Y*EU | S | S | S | O | S | O | OEt | OMe |
| 4286a-l | Y*EU | O | O | O | S | O | S | OH | OMe |
| 4287a-l | Y*EU | O | O | O | S | O | S | OMe | OMe |
| 4288a-l | Y*EU | O | O | O | S | O | S | H | OMe |
| 4289a-l | Y*EU | O | O | O | S | O | S | F | OMe |
| 4290a-l | Y*EU | O | O | O | S | O | S | OEt | OMe |
| 4291a-l | Y*EU | S | O | O | S | O | S | OH | OMe |
| 4292a-l | Y*EU | S | O | O | S | O | S | OMe | OMe |
| 4293a-l | Y*EU | S | O | O | S | O | S | H | OMe |
| 4294a-l | Y*EU | S | O | O | S | O | S | F | OMe |
| 4295a-l | Y*EU | S | O | O | S | O | S | OEt | OMe |
| 4296a-l | Y*EU | S | S | O | S | O | S | OH | OMe |
| 4297a-l | Y*EU | S | S | O | S | O | S | OMe | OMe |
| 4298a-l | Y*EU | S | S | O | S | O | S | H | OMe |
| 4299a-l | Y*EU | S | S | O | S | O | S | F | OMe |
| 4300a-l | Y*EU | S | S | O | S | O | S | OEt | OMe |
| 4301a-l | Y*EI | O | O | O | O | O | O | OH | OH |
| 4302a-l | Y*EI | O | O | O | O | O | O | OMe | OH |
| 4303a-l | Y*EI | O | O | O | O | O | O | H | OH |
| 4304a-l | Y*EI | O | O | O | O | O | O | F | OH |
| 4305a-l | Y*EI | O | O | O | O | O | O | OEt | OH |
| 4306a-l | Y*EI | S | O | O | O | O | O | OH | OH |
| 4307a-l | Y*EI | S | O | O | O | O | O | OMe | OH |
| 4308a-l | Y*EI | S | O | O | O | O | O | H | OH |
| 4309a-l | Y*EI | S | O | O | O | O | O | F | OH |
| 4310a-l | Y*EI | S | O | O | O | O | O | OEt | OH |
| 4311a-l | Y*EI | S | S | O | O | O | O | OF | OH |
| 4312a-l | Y*EI | S | S | O | O | O | O | OMe | OH |
| 4313a-l | Y*EI | S | S | O | O | O | O | H | OH |
| 4314a-l | Y*EI | S | S | O | O | O | O | F | OH |
| 4315a-l | Y*EI | S | S | O | O | O | O | OEt | OH |
| 4316a-l | Y*EI | O | O | S | O | O | O | OH | OH |
| 4317a-l | Y*EI | O | O | S | O | O | O | OMe | OH |
| 4318a-l | Y*EI | O | O | S | O | O | O | H | OH |
| 4319a-l | Y*EI | O | O | S | O | O | O | F | OH |
| 4320a-l | Y*EI | O | O | S | O | O | O | OEt | OH |
| 4321a-l | Y*EI | S | O | S | O | O | O | OH | OH |
| 4322a-l | Y*EI | S | O | S | O | O | O | OMe | OH |
| 4323a-l | Y*EI | S | O | S | O | O | O | H | OH |
| 4324a-l | Y*EI | S | O | S | O | O | O | F | OH |
| 4325a-l | Y*EI | S | O | S | O | O | O | OEt | OH |
| 4326a-l | Y*EI | S | S | S | O | O | O | OH | OH |
| 4327a-l | Y*EI | S | S | S | O | O | O | OMe | OH |
| 4328a-l | Y*EI | S | S | S | O | O | O | H | OH |
| 4329a-l | Y*EI | S | S | S | O | O | O | F | OH |
| 4330a-l | Y*EI | S | S | S | O | O | O | OEt | OH |
| 4331a-l | Y*EI | O | O | S | S | O | O | OH | OH |
| 4332a-l | Y*EI | O | O | S | S | O | O | OMe | OH |
| 4333a-l | Y*EI | O | O | S | S | O | O | H | OH |
| 4334a-l | Y*EI | O | O | S | S | O | O | F | OH |
| 4335a-l | Y*EI | O | O | S | S | O | O | OEt | OH |
| 4336a-l | Y*EI | S | O | S | S | O | O | OH | OH |
| 4337a-l | Y*EI | S | O | S | S | O | O | OMe | OH |
| 4338a-l | Y*EI | S | O | S | S | O | O | H | OH |
| 4339a-l | Y*EI | S | O | S | S | O | O | F | OH |
| 4340a-l | Y*EI | S | O | S | S | O | O | OEt | OH |
| 4341a-l | Y*EI | S | S | S | S | O | O | OH | OH |
| 4342a-l | Y*EI | S | S | S | S | O | O | OMe | OH |
| 4343a-l | Y*EI | S | S | S | S | O | O | H | OH |
| 4344a-l | Y*EI | S | S | S | S | O | O | F | OH |
| 4345a-l | Y*EI | S | S | S | S | O | O | OEt | OH |
| 4346a-l | Y*EI | O | O | S | S | S | O | OH | OH |
| 4347a-l | Y*EI | O | O | S | S | S | O | OMe | OH |
| 4348a-l | Y*EI | O | O | S | S | S | O | H | OH |
| 4349a-l | Y*EI | O | O | S | S | S | O | F | OH |
| 4350a-l | Y*EI | O | O | S | S | S | O | OEt | OH |
| 4351a-l | Y*EI | S | O | S | S | S | O | OH | OH |
| 4352a-l | Y*EI | S | O | S | S | S | O | OMe | OH |
| 4353a-l | Y*EI | S | O | S | S | S | O | H | OH |
| 4354a-l | Y*EI | S | O | S | S | S | O | F | OH |
| 4355a-l | Y*EI | S | O | S | S | S | O | OEt | OH |
| 4356a-l | Y*EI | S | S | S | S | S | O | OH | OH |
| 4357a-l | Y*EI | S | S | S | S | S | O | OMe | OH |
| 4358a-l | Y*EI | S | S | S | S | S | O | H | OH |
| 4359a-l | Y*EI | S | S | S | S | S | O | F | OH |
| 4360a-l | Y*EI | S | S | S | S | S | O | OEt | OH |
| 4361a-l | Y*EI | O | O | S | S | S | S | OH | OH |
| 4362a-l | Y*EI | O | O | S | S | S | S | OMe | OH |
| 4363a-l | Y*EI | O | O | S | S | S | S | H | OH |
| 4364a-l | Y*EI | O | O | S | S | S | S | F | OH |
| 4365a-l | Y*EI | O | O | S | S | S | S | OEt | OH |
| 4366a-l | Y*EI | S | O | S | S | S | S | OH | OH |
| 4367a-l | Y*EI | S | O | S | S | S | S | OMe | OH |
| 4368a-l | Y*EI | S | O | S | S | S | S | H | OH |
| 4369a-l | Y*EI | S | O | S | S | S | S | F | OH |
| 4370a-l | Y*EI | S | O | S | S | S | S | OEt | OH |
| 4371a-l | Y*EI | S | S | S | S | S | S | OH | OH |
| 4372a-l | Y*EI | S | S | S | S | S | S | OMe | OH |
| 4373a-l | Y*EI | S | S | S | S | S | S | H | OH |
| 4374a-l | Y*EI | S | S | S | S | S | S | F | OH |
| 4375a-l | Y*EI | S | S | S | S | S | S | OEt | OH |
| 4376a-l | Y*EI | O | O | O | S | S | S | OH | OH |
| 4377a-l | Y*EI | O | O | O | S | S | S | OMe | OH |
| 4378a-l | Y*EI | O | O | O | S | S | S | H | OH |
| 4379a-l | Y*EI | O | O | O | S | S | S | F | OH |
| 4380a-l | Y*EI | O | O | O | S | S | S | OEt | OH |
| 4381a-l | Y*EI | S | O | O | S | S | S | OH | OH |
| 4382a-l | Y*EI | S | O | O | S | S | S | OMe | OH |
| 4383a-l | Y*EI | S | O | O | S | S | S | H | OH |
| 4384a-l | Y*EI | S | O | O | S | S | S | F | OH |
| 4385a-l | Y*EI | S | O | O | S | S | S | OEt | OH |
| 4386a-l | Y*EI | S | S | O | S | S | S | OH | OH |
| 4387a-l | Y*EI | S | S | O | S | S | S | OMe | OH |
| 4388a-l | Y*EI | S | S | O | S | S | S | H | OH |
| 4389a-l | Y*EI | S | S | O | S | S | S | F | OH |
| 4390a-l | Y*EI | S | S | O | S | S | S | OEt | OH |
| 4391a-l | Y*EI | O | O | O | O | S | S | OH | OH |
| 4392a-l | Y*EI | O | O | O | O | S | S | OMe | OH |
| 4393a-l | Y*EI | O | O | O | O | S | S | H | OH |
| 4394a-l | Y*EI | O | O | O | O | S | S | F | OH |
| 4395a-l | Y*EI | O | O | O | O | S | S | OEt | OH |
| 4396a-l | Y*EI | S | O | O | O | S | S | OH | OH |
| 4397a-l | Y*EI | S | O | O | O | S | S | OMe | OH |
| 4398a-l | Y*EI | S | O | O | O | S | S | H | OH |
| 4399a-l | Y*EI | S | O | O | O | S | S | F | OH |
| 4400a-l | Y*EI | S | O | O | O | S | S | OEt | OH |
| 4401a-l | Y*EI | S | S | O | O | S | S | OH | OH |
| 4402a-l | Y*EI | S | S | O | O | S | S | OMe | OH |
| 4403a-l | Y*EI | S | S | O | O | S | S | H | OH |
| 4404a-l | Y*EI | S | S | O | O | S | S | F | OH |
| 4405a-l | Y*EI | S | S | O | O | S | S | OEt | OH |
| 4406a-l | Y*EI | O | O | O | O | O | S | OH | OH |
| 4407a-l | Y*EI | O | O | O | O | O | S | OMe | OH |
| 4408a-l | Y*EI | O | O | O | O | O | S | H | OH |
| 4409a-l | Y*EI | O | O | O | O | O | S | F | OH |
| 4410a-l | Y*EI | O | O | O | O | O | S | OEt | OH |
| 4411a-l | Y*EI | S | O | O | O | O | S | OH | OH |
| 4412a-l | Y*EI | S | O | O | O | O | S | OMe | OH |
| 4413a-l | Y*EI | S | O | O | O | O | S | H | OH |
| 4414a-l | Y*EI | S | O | O | O | O | S | F | OH |
| 4415a-l | Y*EI | S | O | O | O | O | S | OEt | OH |
| 4416a-l | Y*EI | S | S | O | O | O | S | OH | OH |
| 4417a-l | Y*EI | S | S | O | O | O | S | OMe | OH |
| 4418a-l | Y*EI | S | S | O | O | O | S | H | OH |
| 4419a-l | Y*EI | S | S | O | O | O | S | F | OH |
| 4420a-l | Y*EI | S | S | O | O | O | S | OEt | OH |
| 4421a-l | Y*EI | O | O | S | O | S | O | OH | OH |
| 4422a-l | Y*EI | O | O | S | O | S | O | OMe | OH |
| 4423a-l | Y*EI | O | O | S | O | S | O | H | OH |
| 4424a-l | Y*EI | O | O | S | O | S | O | F | OH |
| 4425a-l | Y*EI | O | O | S | O | S | O | OEt | OH |
| 4426a-l | Y*EI | S | O | S | O | S | O | OH | OH |
| 4427a-l | Y*EI | S | O | S | O | S | O | OMe | OH |
| 4428a-l | Y*EI | S | O | S | O | S | O | H | OH |
| 4429a-l | Y*EI | S | O | S | O | S | O | F | OH |
| 4430a-l | Y*EI | S | O | S | O | S | O | OEt | OH |
| 4431a-l | Y*EI | S | S | S | O | S | O | OH | OH |
| 4432a-l | Y*EI | S | S | S | O | S | O | OMe | OH |
| 4433a-l | Y*EI | S | S | S | O | S | O | H | OH |
| 4434a-l | Y*EI | S | S | S | O | S | O | F | OH |
| 4435a-l | Y*EI | S | S | S | O | S | O | OEt | OH |
| 4436a-l | Y*EI | O | O | O | S | O | S | OH | OH |
| 4437a-l | Y*EI | O | O | O | S | O | S | OMe | OH |
| 4438a-l | Y*EI | O | O | O | S | O | S | H | OH |
| 4439a-l | Y*EI | O | O | O | S | O | S | F | OH |
| 4440a-l | Y*EI | O | O | O | S | O | S | OEt | OH |
| 4441a-l | Y*EI | S | O | O | S | O | S | OH | OH |
| 4442a-l | Y*EI | S | O | O | S | O | S | OMe | OH |
| 4443a-l | Y*EI | S | O | O | S | O | S | H | OH |
| 4444a-l | Y*EI | S | O | O | S | O | S | F | OH |
| 4445a-l | Y*EI | S | O | O | S | O | S | OEt | OH |
| 4446a-l | Y*EI | S | S | O | S | O | S | OH | OH |
| 4447a-l | Y*EI | S | S | O | S | O | S | OMe | OH |
| 4448a-l | Y*EI | S | S | O | S | O | S | H | OH |
| 4449a-l | Y*EI | S | S | O | S | O | S | F | OH |
| 4450a-l | Y*EI | S | S | O | S | O | S | OEt | OH |
| 4451a-l | Y*EI | O | O | O | O | O | O | OH | OMe |
| 4452a-l | Y*EI | O | O | O | O | O | O | OMe | OMe |
| 4453a-l | Y*EI | O | O | O | O | O | O | H | OMe |
| 4454a-l | Y*EI | O | O | O | O | O | O | F | OMe |
| 4455a-l | Y*EI | O | O | O | O | O | O | OEt | OMe |
| 4456a-l | Y*EI | S | O | O | O | O | O | OH | OMe |
| 4457a-l | Y*EI | S | O | O | O | O | O | OMe | OMe |
| 4458a-l | Y*EI | S | O | O | O | O | O | H | OMe |
| 4459a-l | Y*EI | S | O | O | O | O | O | F | OMe |
| 4460a-l | Y*EI | S | O | O | O | O | O | OEt | OMe |
| 4461a-l | Y*EI | S | S | O | O | O | O | OH | OMe |
| 4462a-l | Y*EI | S | S | O | O | O | O | OMe | OMe |
| 4463a-l | Y*EI | S | S | O | O | O | O | H | OMe |
| 4464a-l | Y*EI | S | S | O | O | O | O | F | OMe |
| 4465a-l | Y*EI | S | S | O | O | O | O | OEt | OMe |
| 4466a-l | Y*EI | O | O | S | O | O | O | OH | OMe |
| 4467a-l | Y*EI | O | O | S | O | O | O | OMe | OMe |
| 4468a-l | Y*EI | O | O | S | O | O | O | H | OMe |
| 4469a-l | Y*EI | O | O | S | O | O | O | F | OMe |
| 4470a-l | Y*EI | O | O | S | O | O | O | OEt | OMe |
| 4471a-l | Y*EI | S | O | S | O | O | O | OH | OMe |
| 4472a-l | Y*EI | S | O | S | O | O | O | OMe | OMe |
| 4473a-l | Y*EI | S | O | S | O | O | O | H | OMe |
| 4474a-l | Y*EI | S | O | S | O | O | O | F | OMe |
| 4475a-l | Y*EI | S | O | S | O | O | O | OEt | OMe |
| 4476a-l | Y*EI | S | S | S | O | O | O | OH | OMe |
| 4477a-l | Y*EI | S | S | S | O | O | O | OMe | OMe |
| 4478a-l | Y*EI | S | S | S | O | O | O | H | OMe |
| 4479a-l | Y*EI | S | S | S | O | O | O | F | OMe |
| 4480a-l | Y*EI | S | S | S | O | O | O | OE | OMe |
| 4481a-l | Y*EI | O | O | S | S | O | O | OH | OMe |
| 4482a-l | Y*EI | O | O | S | S | O | O | OMe | OMe |
| 4483a-l | Y*EI | O | O | S | S | O | O | H | OMe |
| 4484a-l | Y*EI | O | O | S | S | O | O | F | OMe |
| 4485a-l | Y*EI | O | O | S | S | O | O | OEt | OMe |
| 4486a-l | Y*EI | S | O | S | S | O | O | OH | OMe |
| 4487a-l | Y*EI | S | O | S | S | O | O | OMe | OMe |
| 4488a-l | Y*EI | S | O | S | S | O | O | H | OMe |
| 4489a-l | Y*EI | S | O | S | S | O | O | F | OMe |
| 4490a-l | Y*EI | S | O | S | S | O | O | OEt | OMe |
| 4491a-l | Y*EI | S | S | S | S | O | O | OH | OMe |
| 4492a-l | Y*EI | S | S | S | S | O | O | OMe | OMe |
| 4493a-l | Y*EI | S | S | S | S | O | O | H | OMe |
| 4494a-l | Y*EI | S | S | S | S | O | O | F | OMe |
| 4495a-l | Y*EI | S | S | S | S | O | O | OEt | OMe |
| 4496a-l | Y*EI | O | O | S | S | S | O | OH | OMe |
| 4497a-l | Y*EI | O | O | S | S | S | O | OMe | OMe |
| 4498a-l | Y*EI | O | O | S | S | S | O | H | OMe |
| 4499a-l | Y*EI | O | O | S | S | S | O | F | OMe |
| 4500a-l | Y*EI | O | O | S | S | S | O | OEt | OMe |
| 4501a-l | Y*EI | S | O | S | S | S | O | OH | OMe |
| 4502a-l | Y*EI | S | O | S | S | S | O | OMe | OMe |
| 4503a-l | Y*EI | S | O | S | S | S | O | H | OMe |
| 4504a-l | Y*EI | S | O | S | S | S | O | F | OMe |
| 4505a-l | Y*EI | S | O | S | S | S | O | OEt | OMe |
| 4506a-l | Y*EI | S | S | S | S | S | O | OH | OMe |
| 4507a-l | Y*EI | S | S | S | S | S | O | OMe | OMe |
| 4508a-l | Y*EI | S | S | S | S | S | O | H | OMe |
| 4509a-l | Y*EI | S | S | S | S | S | O | F | OMe |
| 4510a-l | Y*EI | S | S | S | S | S | O | OEt | OMe |
| 4511a-l | Y*EI | O | O | S | S | S | S | OH | OMe |
| 4512a-l | Y*EI | O | O | S | S | S | S | OMe | OMe |
| 4513a-l | Y*EI | O | O | S | S | S | S | H | OMe |
| 4514a-l | Y*EI | O | O | S | S | S | S | F | OMe |
| 4515a-l | Y*EI | O | O | S | S | S | S | OEt | OMe |
| 4516a-l | Y*EI | S | O | S | S | S | S | OH | OMe |
| 4517a-l | Y*EI | S | O | S | S | S | S | OMe | OMe |
| 4518a-l | Y*EI | S | O | S | S | S | S | H | OMe |
| 4519a-l | Y*EI | S | O | S | S | S | S | F | OMe |
| 4520a-l | Y*EI | S | O | S | S | S | S | OEt | OMe |
| 4521a-l | Y*EI | S | S | S | S | S | S | OH | OMe |
| 4522a-l | Y*EI | S | S | S | S | S | S | OMe | OMe |
| 4523a-l | Y*EI | S | S | S | S | S | S | H | OMe |
| 4524a-l | Y*EI | S | S | S | S | S | S | F | OMe |
| 4525a-l | Y*EI | S | S | S | S | S | S | OEt | OMe |
| 4526a-l | Y*EI | O | O | O | S | S | S | OH | OMe |
| 4527a-l | Y*EI | O | O | O | S | S | S | OMe | OMe |
| 4528a-l | Y*EI | O | O | O | S | S | S | H | OMe |
| 4529a-l | Y*EI | O | O | O | S | S | S | F | OMe |
| 4530a-l | Y*EI | O | O | O | S | S | S | OEt | OMe |
| 4531a-l | Y*EI | S | O | O | S | S | S | OH | OMe |
| 4532a-l | Y*EI | S | O | O | S | S | S | OMe | OMe |
| 4533a-l | Y*EI | S | O | O | S | S | S | H | OMe |
| 4534a-l | Y*EI | S | O | O | S | S | S | F | OMe |
| 4535a-l | Y*EI | S | O | O | S | S | S | OEt | OMe |
| 4536a-l | Y*EI | S | S | O | S | S | S | OH | OMe |
| 4537a-l | Y*EI | S | S | O | S | S | S | OMe | OMe |
| 4538a-l | Y*EI | S | S | O | S | S | S | H | OMe |
| 4539a-l | Y*EI | S | S | O | S | S | S | F | OMe |
| 4540a-l | Y*EI | S | S | O | S | S | S | OEt | OMe |
| 4541a-l | Y*EI | O | O | O | O | S | S | OH | OMe |
| 4542a-l | Y*EI | O | O | O | O | S | S | OMe | OMe |
| 4543a-l | Y*EI | O | O | O | O | S | S | H | OMe |
| 4544a-l | Y*EI | O | O | O | O | S | S | F | OMe |
| 4545a-l | Y*EI | O | O | O | O | S | S | OEt | OMe |
| 4546a-l | Y*EI | S | O | O | O | S | S | OH | OMe |
| 4547a-l | Y*EI | S | O | O | O | S | S | OMe | OMe |
| 4548a-l | Y*EI | S | O | O | O | S | S | H | OMe |
| 4549a-l | Y*EI | S | O | O | O | S | S | F | OMe |
| 4550a-l | Y*EI | S | O | O | O | S | S | OEt | OMe |
| 4551a-l | Y*EI | S | S | O | O | S | S | OH | OMe |
| 4552a-l | Y*EI | S | S | O | O | S | S | OMe | OMe |
| 4553a-l | Y*EI | S | S | O | O | S | S | H | OMe |
| 4554a-l | Y*EI | S | S | O | O | S | S | F | OMe |
| 4555a-l | Y*EI | S | S | O | O | S | S | OEt | OMe |
| 4556a-l | Y*EI | O | O | O | O | O | S | OH | OMe |
| 4557a-l | Y*EI | O | O | O | O | O | S | OMe | OMe |
| 4558a-l | Y*EI | O | O | O | O | O | S | H | OMe |
| 4559a-l | Y*EI | O | O | O | O | O | S | F | OMe |
| 4560a-l | Y*EI | O | O | O | O | O | S | OEt | OMe |
| 4561a-l | Y*EI | S | O | O | O | O | S | OH | OMe |
| 4562a-l | Y*EI | S | O | O | O | O | S | OMe | OMe |
| 4563a-l | Y*EI | S | O | O | O | O | S | H | OMe |
| 4564a-l | Y*EI | S | O | O | O | O | S | F | OMe |
| 4565a-l | Y*EI | S | O | O | O | O | S | DEt | OMe |
| 4566a-l | Y*EI | S | S | O | O | O | S | OH | OMe |
| 4567a-l | Y*EI | S | S | O | O | O | S | OMe | OMe |
| 4568a-l | Y*EI | S | S | O | O | O | S | H | OMe |
| 4569a-l | Y*EI | S | S | O | O | O | S | F | OMe |
| 4570a-l | Y*EI | S | S | O | O | O | S | OEt | OMe |
| 4571a-l | Y*EI | O | O | S | O | S | O | OH | OMe |
| 4572a-l | Y*EI | O | O | S | O | S | O | OMe | OMe |
| 4573a-l | Y*EI | O | O | S | O | S | O | H | OMe |
| 4574a-l | Y*EI | O | O | S | O | S | O | F | OMe |
| 4575a-l | Y*EI | O | O | S | O | S | O | OEt | OMe |
| 4576a-l | Y*EI | S | O | S | O | S | O | OH | OMe |
| 4577a-l | Y*EI | S | O | S | O | S | O | OMe | OMe |
| 4578a-l | Y*EI | S | O | S | O | S | O | H | OMe |
| 4579a-l | Y*EI | S | O | S | O | S | O | F | OMe |
| 4580a-l | Y*EI | S | O | S | O | S | O | OEt | OMe |
| 4581a-l | Y*EI | S | S | S | O | S | O | OH | OMe |
| 4582a-l | Y*EI | S | S | S | O | S | O | OMe | OMe |
| 4583a-l | Y*EI | S | S | S | O | S | O | H | OMe |
| 4584a-l | Y*EI | S | S | S | O | S | O | F | OMe |
| 4585a-l | Y*EI | S | S | S | O | S | O | OEt | OMe |
| 4586a-l | Y*EI | O | O | O | S | O | S | OH | OMe |
| 4587a-l | Y*EI | O | O | O | S | O | S | OMe | OMe |
| 4588a-l | Y*EI | O | O | O | S | O | S | H | OMe |
| 4589a-l | Y*EI | O | O | O | S | O | S | F | OMe |
| 4590a-l | Y*EI | O | O | O | S | O | S | OEt | OMe |
| 4591a-l | Y*EI | S | O | O | S | O | S | OH | OMe |
| 4592a-l | Y*EI | S | O | O | S | O | S | OMe | OMe |
| 4593a-l | Y*EI | S | O | O | S | O | S | H | OMe |
| 4594a-l | Y*EI | S | O | O | S | O | S | F | OMe |
| 4595a-l | Y*EI | S | O | O | S | O | S | OEt | OMe |
| 4596a-l | Y*EI | S | S | O | S | O | S | OH | OMe |
| 4597a-l | Y*EI | S | S | O | S | O | S | OMe | OMe |
| 4598a-l | Y*EI | S | S | O | S | O | S | H | OMe |
| 4599a-l | Y*EI | S | S | O | S | O | S | F | OMe |
| 4600a-l | Y*EI | S | S | O | S | O | S | OEt | OMe |
| 4601a-l | Y*ET | O | O | O | O | O | O | OH | OH |
| 4602a-l | Y*ET | O | O | O | O | O | O | OMe | OH |
| 4603a-l | Y*ET | O | O | O | O | O | O | H | OH |
| 4604a-l | Y*ET | O | O | O | O | O | O | F | OH |
| 4605a-l | Y*ET | O | O | O | O | O | O | OEt | OH |
| 4606a-l | Y*ET | S | O | O | O | O | O | OH | OH |
| 4607a-l | Y*ET | S | O | O | O | O | O | OMe | OH |
| 4608a-l | Y*ET | S | O | O | O | O | O | H | OH |
| 4609a-l | Y*ET | S | O | O | O | O | O | F | OH |
| 4610a-l | Y*ET | S | O | O | O | O | O | OEt | OH |
| 4611a-l | Y*ET | S | S | O | O | O | O | OH | OH |
| 4612a-l | Y*ET | S | S | O | O | O | O | OMe | OH |
| 4613a-l | Y*ET | S | S | O | O | O | O | H | OH |
| 4614a-l | Y*ET | S | S | O | O | O | O | F | OH |
| 4615a-l | Y*ET | S | S | O | O | O | O | OEt | OH |
| 4616a-l | Y*ET | O | O | S | O | O | O | OH | OH |
| 4617a-l | Y*ET | O | O | S | O | O | O | OMe | OH |
| 4618a-l | Y*ET | O | O | S | O | O | O | H | OH |
| 4619a-l | Y*ET | O | O | S | O | O | O | F | OH |
| 4620a-l | Y*ET | O | O | S | O | O | O | OEt | OH |
| 4621a-l | Y*ET | S | O | S | O | O | O | OH | OH |
| 4622a-l | Y*ET | S | O | S | O | O | O | OMe | OH |
| 4623a-l | Y*ET | S | O | S | O | O | O | H | OH |
| 4624a-l | Y*ET | S | O | S | O | O | O | F | OH |
| 4625a-l | Y*ET | S | O | S | O | O | O | OEt | OH |
| 4626a-l | Y*ET | S | S | S | O | O | O | OH | OH |
| 4627a-l | Y*ET | S | S | S | O | O | O | OMe | OH |
| 4628a-l | Y*ET | S | S | S | O | O | O | H | OH |
| 4629a-l | Y*ET | S | S | S | O | O | O | F | OH |
| 4630a-l | Y*ET | S | S | S | O | O | O | OEt | OH |
| 4631a-l | Y*ET | O | O | S | S | O | O | OH | OH |
| 4632a-l | Y*ET | O | O | S | S | O | O | OMe | OH |
| 4633a-l | Y*ET | O | O | S | S | O | O | H | OH |
| 4634a-l | Y*ET | O | O | S | S | O | O | F | OH |
| 4635a-l | Y*ET | O | O | S | S | O | O | OEt | OH |
| 4636a-l | Y*ET | S | O | S | S | O | O | OH | OH |
| 4637a-l | Y*ET | S | O | S | S | O | O | OMe | OH |
| 4638a-l | Y*ET | S | O | S | S | O | O | H | OH |
| 4639a-l | Y*ET | S | O | S | S | O | O | F | OH |
| 4640a-l | Y*ET | S | O | S | S | O | O | OEt | OH |
| 4641a-l | Y*ET | S | S | S | S | O | O | OH | OH |
| 4642a-l | Y*ET | S | S | S | S | O | O | OMe | OH |
| 4643a-l | Y*ET | S | S | S | S | O | O | H | OH |
| 4644a-l | Y*ET | S | S | S | S | O | O | F | OH |
| 4645a-l | Y*ET | S | S | S | S | O | O | OEt | OH |
| 4646a-l | Y*ET | O | O | S | S | S | O | OH | OH |
| 4647a-l | Y*ET | O | O | S | S | S | O | OMe | OH |
| 4648a-l | Y*ET | O | O | S | S | S | O | H | OH |
| 4649a-l | Y*ET | O | O | S | S | S | O | F | OH |
| 4650a-l | Y*ET | O | O | S | S | S | O | OEt | OH |
| 4651a-l | Y*ET | S | O | S | S | S | O | OH | OH |
| 4652a-l | Y*ET | S | O | S | S | S | O | OMe | OH |
| 4653a-l | Y*ET | S | O | S | S | S | O | H | OH |
| 4654a-l | Y*ET | S | O | S | S | S | O | F | OH |
| 4655a-l | Y*ET | S | O | S | S | S | O | OEt | OH |
| 4656a-l | Y*ET | S | S | S | S | S | O | OH | OH |
| 4657a-l | Y*ET | S | S | S | S | S | O | OMe | OH |
| 4658a-l | Y*ET | S | S | S | S | S | O | H | OH |
| 4659a-l | Y*ET | S | S | S | S | S | O | F | OH |
| 4660a-l | Y*ET | S | S | S | S | S | O | OEt | OH |
| 4661a-l | Y*ET | O | O | S | S | S | S | OH | OH |
| 4662a-l | Y*ET | O | O | S | S | S | S | OMe | OH |
| 4663a-l | Y*ET | O | O | S | S | S | S | H | OH |
| 4664a-l | Y*ET | O | O | S | S | S | S | F | OH |
| 4665a-l | Y*ET | O | O | S | S | S | S | OEt | OH |
| 4666a-l | Y*ET | S | O | S | S | S | S | OH | OH |
| 4667a-l | Y*ET | S | O | S | S | S | S | OMe | OH |
| 4668a-l | Y*ET | S | O | S | S | S | S | H | OH |
| 4669a-l | Y*ET | S | O | S | S | S | S | F | OH |
| 4670a-l | Y*ET | S | O | S | S | S | S | OEt | OH |
| 4671a-l | Y*ET | S | S | S | S | S | S | OH | OH |
| 4672a-l | Y*ET | S | S | S | S | S | S | OMe | OH |
| 4673a-l | Y*ET | S | S | S | S | S | S | H | OH |
| 4674a-l | Y*ET | S | S | S | S | S | S | F | OH |
| 4675a-l | Y*ET | S | S | S | S | S | S | OEt | OH |
| 4676a-l | Y*ET | O | O | O | S | S | S | OH | OH |
| 4677a-l | Y*ET | O | O | O | S | S | S | OMe | OH |
| 4678a-l | Y*ET | O | O | O | S | S | S | H | OH |
| 4679a-l | Y*ET | O | O | O | S | S | S | F | OH |
| 4680a-l | Y*ET | O | O | O | S | S | S | OEt | OH |
| 4681a-l | Y*ET | S | O | O | S | S | S | OH | OH |
| 4682a-l | Y*ET | S | O | O | S | S | S | OMe | OH |
| 4683a-l | Y*ET | S | O | O | S | S | S | H | OH |
| 4684a-l | Y*ET | S | O | O | S | S | S | F | OH |
| 4685a-l | Y*ET | S | O | O | S | S | S | OEt | OH |
| 4686a-l | Y*ET | S | S | O | S | S | S | OH | OH |
| 4687a-l | Y*ET | S | S | O | S | S | S | OMe | OH |
| 4688a-l | Y*ET | S | S | O | S | S | S | H | OH |
| 4689a-l | Y*ET | S | S | O | S | S | S | F | OH |
| 4690a-l | Y*ET | S | S | O | S | S | S | OEt | OH |
| 4691a-l | Y*ET | O | O | O | O | S | S | OH | OH |
| 4692a-l | Y*ET | O | O | O | O | S | S | OMe | OH |
| 4693a-l | Y*ET | O | O | O | O | S | S | H | OH |
| 4694a-l | Y*ET | O | O | O | O | S | S | F | OH |
| 4695a-l | Y*ET | O | O | O | O | S | S | OEt | OH |
| 4696a-l | Y*ET | S | O | O | O | S | S | OH | OH |
| 4697a-l | Y*ET | S | O | O | O | S | S | OMe | OH |
| 4698a-l | Y*ET | S | O | O | O | S | S | H | OH |
| 4699a-l | Y*ET | S | O | O | O | S | S | F | OH |
| 4700a-l | Y*ET | S | O | O | O | S | S | OEt | OH |
| 4701a-l | Y*ET | S | S | O | O | S | S | OH | OH |
| 4702a-l | Y*ET | S | S | O | O | S | S | OMe | OH |
| 4703a-l | Y*ET | S | S | O | O | S | S | H | OH |
| 4704a-l | Y*ET | S | S | O | O | S | S | F | OH |
| 4705a-l | Y*ET | S | S | O | O | S | S | OEt | OH |
| 4706a-l | Y*ET | O | O | O | O | O | S | OH | OH |
| 4707a-l | Y*ET | O | O | O | O | O | S | OMe | OH |
| 4708a-l | Y*ET | O | O | O | O | O | S | H | OH |
| 4709a-l | Y*ET | O | O | O | O | O | S | F | OH |
| 4710a-l | Y*ET | O | O | O | O | O | S | OEt | OH |
| 4711a-l | Y*ET | S | O | O | O | O | S | OH | OH |
| 4712a-l | Y*ET | S | O | O | O | O | S | OMe | OH |
| 4713a-l | Y*ET | S | O | O | O | O | S | H | OH |
| 4714a-l | Y*ET | S | O | O | O | O | S | F | OH |
| 4715a-l | Y*ET | S | O | O | O | O | S | OEt | OH |
| 4716a-l | Y*ET | S | S | O | O | O | S | OH | OH |
| 4717a-l | Y*ET | S | S | O | O | O | S | OMe | OH |
| 4718a-l | Y*ET | S | S | O | O | O | S | H | OH |
| 4719a-l | Y*ET | S | S | O | O | O | S | F | OH |
| 4720a-l | Y*ET | S | S | O | O | O | S | OEt | OH |
| 4721a-l | Y*ET | O | O | S | O | S | O | OH | OH |
| 4722a-l | Y*ET | O | O | S | O | S | O | OMe | OH |
| 4723a-l | Y*ET | O | O | S | O | S | O | H | OF |
| 4724a-l | Y*ET | O | O | S | O | S | O | F | OH |
| 4725a-l | Y*ET | O | O | S | O | S | O | OEt | OH |
| 4726a-l | Y*ET | S | O | S | O | S | O | OH | OH |
| 4727a-l | Y*ET | S | O | S | O | S | O | OMe | OH |
| 4728a-l | Y*ET | S | O | S | O | S | O | H | OH |
| 4729a-l | Y*ET | S | O | S | O | S | O | F | OF |
| 4730a-l | Y*ET | S | O | S | O | S | O | OEt | OH |
| 4731a-l | Y*ET | S | S | S | O | S | O | OH | OH |
| 4732a-l | Y*ET | S | S | S | O | S | O | OMe | OH |
| 4733a-l | Y*ET | S | S | S | O | S | O | H | OH |
| 4734a-l | Y*ET | S | S | S | O | S | O | F | OH |
| 4735a-l | Y*ET | S | S | S | O | S | O | OEt | OH |
| 4736a-l | Y*ET | O | O | O | S | O | S | OH | OH |
| 4737a-l | Y*ET | O | O | O | S | O | S | OMe | OH |
| 4738a-l | Y*ET | O | O | O | S | O | S | H | OH |
| 4739a-l | Y*ET | O | O | O | S | O | S | F | OH |
| 4740a-l | Y*ET | O | O | O | S | O | S | OEt | OH |
| 4741a-l | Y*ET | S | O | O | S | O | S | OH | OH |
| 4742a-l | Y*ET | S | O | O | S | O | S | OMe | OH |
| 4743a-l | Y*ET | S | O | O | S | O | S | H | OH |
| 4744a-l | Y*ET | S | O | O | S | O | S | F | OH |
| 4745a-l | Y*ET | S | O | O | S | O | S | OEt | OH |
| 4746a-l | Y*ET | S | S | O | S | O | S | OH | OH |
| 4747a-l | Y*ET | S | S | O | S | O | S | OMe | OH |
| 4748a-l | Y*ET | S | S | O | S | O | S | H | OH |
| 4749a-l | Y*ET | S | S | O | S | O | S | F | OH |
| 4750a-l | Y*ET | S | S | O | S | O | S | OEt | OH |
| 4751a-l | Y*ET | O | O | O | O | O | O | OH | OMe |
| 4752a-l | Y*ET | O | O | O | O | O | O | OMe | OMe |
| 4753a-l | Y*ET | O | O | O | O | O | O | H | OMe |
| 4754a-l | Y*ET | O | O | O | O | O | O | F | OMe |
| 4755a-l | Y*ET | O | O | O | O | O | O | OEt | OMe |
| 4756a-l | Y*ET | S | O | O | O | O | O | OH | OMe |
| 4757a-l | Y*ET | S | O | O | O | O | O | OMe | OMe |
| 4758a-l | Y*ET | S | O | O | O | O | O | H | OMe |
| 4759a-l | Y*ET | S | O | O | O | O | O | F | OMe |
| 4760a-l | Y*ET | S | O | O | O | O | O | OEt | OMe |
| 4761a-l | Y*ET | S | S | O | O | O | O | OH | OMe |
| 4762a-l | Y*ET | S | S | O | O | O | O | OMe | OMe |
| 4763a-l | Y*ET | S | S | O | O | O | O | H | OMe |
| 4764a-l | Y*ET | S | S | O | O | O | O | F | OMe |
| 4765a-l | Y*ET | S | S | O | O | O | O | OEt | OMe |
| 4766a-l | Y*ET | O | O | S | O | O | O | OH | OMe |
| 4767a-l | Y*ET | O | O | S | O | O | O | OMe | OMe |
| 4768a-l | Y*ET | O | O | S | O | O | O | H | OMe |
| 4769a-l | Y*ET | O | O | S | O | O | O | F | OMe |
| 4770a-l | Y*ET | O | O | S | O | O | O | OEt | OMe |
| 4771a-l | Y*ET | S | O | S | O | O | O | OH | OMe |
| 4772a-l | Y*ET | S | O | S | O | O | O | OMe | OMe |
| 4773a-l | Y*ET | S | O | S | O | O | O | H | OMe |
| 4774a-l | Y*ET | S | O | S | O | O | O | F | OMe |
| 4775a-l | Y*ET | S | O | S | O | O | O | OEt | OMe |
| 4776a-l | Y*ET | S | S | S | O | O | O | OH | OMe |
| 4777a-l | Y*ET | S | S | S | O | O | O | OMe | OMe |
| 4778a-l | Y*ET | S | S | S | O | O | O | H | OMe |
| 4779a-l | Y*ET | S | S | S | O | O | O | F | OMe |
| 4780a-l | Y*ET | S | S | S | O | O | O | OEt | OMe |
| 4781a-l | Y*ET | O | O | S | S | O | O | OH | OMe |
| 4782a-l | Y*ET | O | O | S | S | O | O | OMe | OMe |
| 4783a-l | Y*ET | O | O | S | S | O | O | H | OMe |
| 4784a-l | Y*ET | O | O | S | S | O | O | F | OMe |
| 4785a-l | Y*ET | O | O | S | S | O | O | OEt | OMe |
| 4786a-l | Y*ET | S | O | S | S | O | O | OH | OMe |
| 4787a-l | Y*ET | S | O | S | S | O | O | OMe | OMe |
| 4788a-l | Y*ET | S | O | S | S | O | O | H | OMe |
| 4789a-l | Y*ET | S | O | S | S | O | O | F | OMe |
| 4790a-l | Y*ET | S | O | S | S | O | O | OEt | OMe |
| 4791a-l | Y*ET | S | S | S | S | O | O | OH | OMe |
| 4792a-l | Y*ET | S | S | S | S | O | O | OMe | OMe |
| 4793a-l | Y*ET | S | S | S | S | O | O | H | OMe |
| 4794a-l | Y*ET | S | S | S | S | O | O | F | OMe |
| 4795a-l | Y*ET | S | S | S | S | O | O | OEt | OMe |
| 4796a-l | Y*ET | O | O | S | S | S | O | OH | OMe |
| 4797a-l | Y*ET | O | O | S | S | S | O | OMe | OMe |
| 4798a-l | Y*ET | O | O | S | S | S | O | H | OMe |
| 4799a-l | Y*ET | O | O | S | S | S | O | F | OMe |
| 4800a-l | Y*ET | O | O | S | S | S | O | OEt | OMe |
| 4801a-l | Y*ET | S | O | S | S | S | O | OH | OMe |
| 4802a-l | Y*ET | S | O | S | S | S | O | OMe | OMe |
| 4803a-l | Y*ET | S | O | S | S | S | O | H | OMe |
| 4804a-l | Y*ET | S | O | S | S | S | O | F | OMe |
| 4805a-l | Y*ET | S | O | S | S | S | O | OEt | OMe |
| 4806a-l | Y*ET | S | S | S | S | S | O | OH | OMe |
| 4807a-l | Y*ET | S | S | S | S | S | O | OMe | OMe |
| 4808a-l | Y*ET | S | S | S | S | S | O | H | OMe |
| 4809a-l | Y*ET | S | S | S | S | S | O | F | OMe |
| 4810a-l | Y*ET | S | S | S | S | S | O | OEt | OMe |
| 4811a-l | Y*ET | O | O | S | S | S | S | OH | OMe |
| 4812a-l | Y*ET | O | O | S | S | S | S | OMe | OMe |
| 4813a-l | Y*ET | O | O | S | S | S | S | H | OMe |
| 4814a-l | Y*ET | O | O | S | S | S | S | F | OMe |
| 4815a-l | Y*ET | O | O | S | S | S | S | OEt | OMe |
| 4816a-l | Y*ET | S | O | S | S | S | S | OH | OMe |
| 4817a-l | Y*ET | S | O | S | S | S | S | OMe | OMe |
| 4818a-l | Y*ET | S | O | S | S | S | S | H | OMe |
| 4819a-l | Y*ET | S | O | S | S | S | S | F | OMe |
| 4820a-l | Y*ET | S | O | S | S | S | S | OEt | OMe |
| 4821a-l | Y*ET | S | S | S | S | S | S | OH | OMe |
| 4822a-l | Y*ET | S | S | S | S | S | S | OMe | OMe |
| 4823a-l | Y*ET | S | S | S | S | S | S | H | OMe |
| 4824a-l | Y*ET | S | S | S | S | S | S | F | OMe |
| 4825a-l | Y*ET | S | S | S | S | S | S | OEt | OMe |
| 4826a-l | Y*ET | O | O | O | S | S | S | OH | OMe |
| 4827a-l | Y*ET | O | O | O | S | S | S | OMe | OMe |
| 4828a-l | Y*ET | O | O | O | S | S | S | H | OMe |
| 4829a-l | Y*ET | O | O | O | S | S | S | F | OMe |
| 4830a-l | Y*ET | O | O | O | S | S | S | OEt | OMe |
| 4831a-l | Y*ET | S | O | O | S | S | S | OH | OMe |
| 4832a-l | Y*ET | S | O | O | S | S | S | OMe | OMe |
| 4833a-l | Y*ET | S | O | O | S | S | S | H | OMe |
| 4834a-l | Y*ET | S | O | O | S | S | S | F | OMe |
| 4835a-l | Y*ET | S | O | O | S | S | S | OEt | OMe |
| 4836a-l | Y*ET | S | S | O | S | S | S | OH | OMe |
| 4837a-l | Y*ET | S | S | O | S | S | S | OMe | OMe |
| 4838a-l | Y*ET | S | S | O | S | S | S | H | OMe |
| 4839a-l | Y*ET | S | S | O | S | S | S | F | OMe |
| 4840a-l | Y*ET | S | S | O | S | S | S | OEt | OMe |
| 4841a-l | Y*ET | O | O | O | O | S | S | OH | OMe |
| 4842a-l | Y*ET | O | O | O | O | S | S | OMe | OMe |
| 4843a-l | Y*ET | O | O | O | O | S | S | H | OMe |
| 4844a-l | Y*ET | O | O | O | O | S | S | F | OMe |
| 4845a-l | Y*ET | O | O | O | O | S | S | OEt | OMe |
| 4846a-l | Y*ET | S | O | O | O | S | S | OH | OMe |
| 4847a-l | Y*ET | S | O | O | O | S | S | OMe | OMe |
| 4848a-l | Y*ET | S | O | O | O | S | S | H | OMe |
| 4849a-l | Y*ET | S | O | O | O | S | S | F | OMe |
| 4850a-l | Y*ET | S | O | O | O | S | S | OEt | OMe |
| 4851a-l | Y*ET | S | S | O | O | S | S | OH | OMe |
| 4852a-l | Y*ET | S | S | O | O | S | S | OMe | OMe |
| 4853a-l | Y*ET | S | S | O | O | S | S | H | OMe |
| 4854a-l | Y*ET | S | S | O | O | S | S | F | OMe |
| 4855a-l | Y*ET | S | S | O | O | S | S | OEt | OMe |
| 4856a-l | Y*ET | O | O | O | O | O | S | OH | OMe |
| 4857a-l | Y*ET | O | O | O | O | O | S | OMe | OMe |
| 4858a-l | Y*ET | O | O | O | O | O | S | H | OMe |
| 4859a-l | Y*ET | O | O | O | O | O | S | F | OMe |
| 4860a-l | Y*ET | O | O | O | O | O | S | OEt | OMe |
| 4861a-l | Y*ET | S | O | O | O | O | S | OH | OMe |
| 4862a-l | Y*ET | S | O | O | O | O | S | OMe | OMe |
| 4863a-l | Y*ET | S | O | O | O | O | S | H | OMe |
| 4864a-l | Y*ET | S | O | O | O | O | S | F | OMe |
| 4865a-l | Y*ET | S | O | O | O | O | S | OEt | OMe |
| 4866a-l | Y*ET | S | S | O | O | O | S | OH | OMe |
| 4867a-l | Y*ET | S | S | O | O | O | S | OMe | OMe |
| 4868a-l | Y*ET | S | S | O | O | O | S | H | OMe |
| 4869a-l | Y*ET | S | S | O | O | O | S | F | OMe |
| 4870a-l | Y*ET | S | S | O | O | O | S | OEt | OMe |
| 4871a-l | Y*ET | O | O | S | O | S | O | OH | OMe |
| 4872a-l | Y*ET | O | O | S | O | S | O | OMe | OMe |
| 4873a-l | Y*ET | O | O | S | O | S | O | H | OMe |
| 4874a-l | Y*ET | O | O | S | O | S | O | F | OMe |
| 4875a-l | Y*ET | O | O | S | O | S | O | OEt | OMe |
| 4876a-l | Y*ET | S | O | S | O | S | O | OH | OMe |
| 4877a-l | Y*ET | S | O | S | O | S | O | OMe | OMe |
| 4878a-l | Y*ET | S | O | S | O | S | O | H | OMe |
| 4879a-l | Y*ET | S | O | S | O | S | O | F | OMe |
| 4880a-l | Y*ET | S | O | S | O | S | O | OEt | OMe |
| 4881a-l | Y*ET | S | S | S | O | S | O | OH | OMe |
| 4882a-l | Y*ET | S | S | S | O | S | O | OMe | OMe |
| 4883a-l | Y*ET | S | S | S | O | S | O | H | OMe |
| 4884a-l | Y*ET | S | S | S | O | S | O | F | OMe |
| 4885a-l | Y*ET | S | S | S | O | S | O | OEt | OMe |
| 4886a-l | Y*ET | O | O | O | S | O | S | OH | OMe |
| 4887a-l | Y*ET | O | O | O | S | O | S | OMe | OMe |
| 4888a-l | Y*ET | O | O | O | S | O | S | H | OMe |
| 4889a-l | Y*ET | O | O | O | S | O | S | F | OMe |
| 4890a-l | Y*ET | O | O | O | S | O | S | OEt | OMe |
| 4891a-l | Y*ET | S | O | O | S | O | S | OH | OMe |
| 4892a-l | Y*ET | S | O | O | S | O | S | OMe | OMe |
| 4893a-l | Y*ET | S | O | O | S | O | S | H | OMe |
| 4894a-l | Y*ET | S | O | O | S | O | S | F | OMe |
| 4895a-l | Y*ET | S | O | O | S | O | S | OEt | OMe |
| 4896a-l | Y*ET | S | S | O | S | O | S | OH | OMe |
| 4897a-l | Y*ET | S | S | O | S | O | S | OMe | OMe |
| 4898a-l | Y*ET | S | S | O | S | O | S | H | OMe |
| 4899a-l | Y*ET | S | S | O | S | O | S | F | OMe |
| 4900a-l | Y*ET | S | S | O | S | O | S | OEt | OMe |
| 4901a-l | Y*EE | O | O | O | O | O | O | OH | OH |
| 4902a-l | Y*EE | O | O | O | O | O | O | OMe | OH |
| 4903a-l | Y*EE | O | O | O | O | O | O | H | OH |
| 4904a-l | Y*EE | O | O | O | O | O | O | F | OH |
| 4905a-l | Y*EE | O | O | O | O | O | O | OEt | OH |
| 4906a-l | Y*EE | S | O | O | O | O | O | OH | OH |
| 4907a-l | Y*EE | S | O | O | O | O | O | OMe | OH |
| 4908a-l | Y*EE | S | O | O | O | O | O | H | OH |
| 4909a-l | Y*EE | S | O | O | O | O | O | F | OH |
| 4910a-l | Y*EE | S | O | O | O | O | O | OEt | OH |
| 4911a-l | Y*EE | S | S | O | O | O | O | OH | OH |
| 4912a-l | Y*EE | S | S | O | O | O | O | OMe | OH |
| 4913a-l | Y*EE | S | S | O | O | O | O | H | OH |
| 4914a-l | Y*EE | S | S | O | O | O | O | F | OH |
| 4915a-l | Y*EE | S | S | O | O | O | O | OEt | OH |
| 4916a-l | Y*EE | O | O | S | O | O | O | OH | OH |
| 4917a-l | Y*EE | O | O | S | O | O | O | OMe | OH |
| 4918a-l | Y*EE | O | O | S | O | O | O | H | OH |
| 4919a-l | Y*EE | O | O | S | O | O | O | F | OH |
| 4920a-l | Y*EE | O | O | S | O | O | O | OEt | OH |
| 4921a-l | Y*EE | S | O | S | O | O | O | OH | OH |
| 4922a-l | Y*EE | S | O | S | O | O | O | OMe | OH |
| 4923a-l | Y*EE | S | O | S | O | O | O | H | OH |
| 4924a-l | Y*EE | S | O | S | O | O | O | F | OH |
| 4925a-l | Y*EE | S | O | S | O | O | O | OEt | OH |
| 4926a-l | Y*EE | S | S | S | O | O | O | OH | OH |
| 4927a-l | Y*EE | S | S | S | O | O | O | OMe | OH |
| 4928a-l | Y*EE | S | S | S | O | O | O | H | OH |
| 4929a-l | Y*EE | S | S | S | O | O | O | F | OH |
| 4930a-l | Y*EE | S | S | S | O | O | O | OEt | OH |
| 4931a-l | Y*EE | O | O | S | S | O | O | OH | OH |
| 4932a-l | Y*EE | O | O | S | S | O | O | OMe | OH |
| 4933a-l | Y*EE | O | O | S | S | O | O | H | OH |
| 4934a-l | Y*EE | O | O | S | S | O | O | F | OH |
| 4935a-l | Y*EE | O | O | S | S | O | O | OEt | OH |
| 4936a-l | Y*EE | S | O | S | S | O | O | OH | OH |
| 4937a-l | Y*EE | S | O | S | S | O | O | OMe | OH |
| 4938a-l | Y*EE | S | O | S | S | O | O | H | OH |
| 4939a-l | Y*EE | S | O | S | S | O | O | F | OH |
| 4940a-l | Y*EE | S | O | S | S | O | O | OEt | OH |
| 4941a-l | Y*EE | S | S | S | S | O | O | OH | OH |
| 4942a-l | Y*EE | S | S | S | S | O | O | OMe | OH |
| 4943a-l | Y*EE | S | S | S | S | O | O | H | OH |
| 4944a-l | Y*EE | S | S | S | S | O | O | F | OH |
| 4945a-l | Y*EE | S | S | S | S | O | O | OEt | OH |
| 4946a-l | Y*EE | O | O | S | S | S | O | OH | OH |
| 4947a-l | Y*EE | O | O | S | S | S | O | OMe | OH |
| 4948a-l | Y*EE | O | O | S | S | S | O | H | OH |
| 4949a-l | Y*EE | O | O | S | S | S | O | F | OH |
| 4950a-l | Y*EE | O | O | S | S | S | O | OEt | OH |
| 4951a-l | Y*EE | S | O | S | S | S | O | OH | OH |
| 4952a-l | Y*EE | S | O | S | S | S | O | OMe | OH |
| 4953a-l | Y*EE | S | O | S | S | S | O | H | OH |
| 4954a-l | Y*EE | S | O | S | S | S | O | F | OH |
| 4955a-l | Y*EE | S | O | S | S | S | O | OEt | OH |
| 4956a-l | Y*EE | S | S | S | S | S | O | OH | OH |
| 4957a-l | Y*EE | S | S | S | S | S | O | OMe | OH |
| 4958a-l | Y*EE | S | S | S | S | S | O | H | OH |
| 4959a-l | Y*EE | S | S | S | S | S | O | F | OH |
| 4960a-l | Y*EE | S | S | S | S | S | O | OEt | OH |
| 4961a-l | Y*EE | O | O | S | S | S | S | OH | OH |
| 4962a-l | Y*EE | O | O | S | S | S | S | OMe | OH |
| 4963a-l | Y*EE | O | O | S | S | S | S | H | OH |
| 4964a-l | Y*EE | O | O | S | S | S | S | F | OH |
| 4965a-l | Y*EE | O | O | S | S | S | S | OEt | OH |
| 4966a-l | Y*EE | S | O | S | S | S | S | OH | OH |
| 4967a-l | Y*EE | S | O | S | S | S | S | OMe | OH |
| 4968a-l | Y*EE | S | O | S | S | S | S | H | OH |
| 4969a-l | Y*EE | S | O | S | S | S | S | F | OH |
| 4970a-l | Y*EE | S | O | S | S | S | S | OEt | OH |
| 4971a-l | Y*EE | S | S | S | S | S | S | OH | OH |
| 4972a-l | Y*EE | S | S | S | S | S | S | OMe | OH |
| 4973a-l | Y*EE | S | S | S | S | S | S | H | OH |
| 4974a-l | Y*EE | S | S | S | S | S | S | F | OH |
| 4975a-l | Y*EE | S | S | S | S | S | S | OEt | OH |
| 4976a-l | Y*EE | O | O | O | S | S | S | OH | OH |
| 4977a-l | Y*EE | O | O | O | S | S | S | OMe | OH |
| 4978a-l | Y*EE | O | O | O | S | S | S | H | OH |
| 4979a-l | Y*EE | O | O | O | S | S | S | F | OH |
| 4980a-l | Y*EE | O | O | O | S | S | S | OEt | OH |
| 4981a-l | Y*EE | S | O | O | S | S | S | OH | OH |
| 4982a-l | Y*EE | S | O | O | S | S | S | OMe | OH |
| 4983a-l | Y*EE | S | O | O | S | S | S | H | OH |
| 4984a-l | Y*EE | S | O | O | S | S | S | F | OH |
| 4985a-l | Y*EE | S | O | O | S | S | S | OEt | OH |
| 4986a-l | Y*EE | S | S | O | S | S | S | OH | OH |
| 4987a-l | Y*EE | S | S | O | S | S | S | OMe | OH |
| 4988a-l | Y*EE | S | S | O | S | S | S | H | OH |
| 4989a-l | Y*EE | S | S | O | S | S | S | F | OH |
| 4990a-l | Y*EE | S | S | O | S | S | S | OEt | OH |
| 4991a-l | Y*EE | O | O | O | O | S | S | OH | OH |
| 4992a-l | Y*EE | O | O | O | O | S | S | OMe | OH |
| 4993a-l | Y*EE | O | O | O | O | S | S | H | OH |
| 4994a-l | Y*EE | O | O | O | O | S | S | F | OH |
| 4995a-l | Y*EE | O | O | O | O | S | S | OEt | OH |
| 4996a-l | Y*EE | S | O | O | O | S | S | OH | OH |
| 4997a-l | Y*EE | S | O | O | O | S | S | OMe | OH |
| 4998a-l | Y*EE | S | O | O | O | S | S | H | OH |
| 4999a-l | Y*EE | S | O | O | O | S | S | F | OH |
| 5000a-l | Y*EE | S | O | O | O | S | S | OEt | OH |
| 5001a-l | Y*EE | S | S | O | O | S | S | OH | OH |
| 5002a-l | Y*EE | S | S | O | O | S | S | OMe | OH |
| 5003a-l | Y*EE | S | S | O | O | S | S | H | OH |
| 5004a-l | Y*EE | S | S | O | O | S | S | F | OH |
| 5005a-l | Y*EE | S | S | O | O | S | S | OEt | OH |
| 5006a-l | Y*EE | O | O | O | O | O | S | OH | OH |
| 5007a-l | Y*EE | O | O | O | O | O | S | OMe | OH |
| 5008a-l | Y*EE | O | O | O | O | O | S | H | OH |
| 5009a-l | Y*EE | O | O | O | O | O | S | F | OH |
| 5010a-l | Y*EE | O | O | O | O | O | S | OEt | OH |
| 5011a-l | Y*EE | S | O | O | O | O | S | OH | OH |
| 5012a-l | Y*EE | S | O | O | O | O | S | OMe | OH |
| 5013a-l | Y*EE | S | O | O | O | O | S | H | OH |
| 5014a-l | Y*EE | S | O | O | O | O | S | F | OH |
| 5015a-l | Y*EE | S | O | O | O | O | S | OEt | OH |
| 5016a-l | Y*EE | S | S | O | O | O | S | OH | OH |
| 5017a-l | Y*EE | S | S | O | O | O | S | OMe | OH |
| 5018a-l | Y*EE | S | S | O | O | O | S | H | OH |
| 5019a-l | Y*EE | S | S | O | O | O | S | F | OH |
| 5020a-l | Y*EE | S | S | O | O | O | S | OEt | OH |
| 5021a-l | Y*EE | O | O | S | O | S | O | OH | OH |
| 5022a-l | Y*EE | O | O | S | O | S | O | OMe | OH |
| 5023a-l | Y*EE | O | O | S | O | S | O | H | OH |
| 5024a-l | Y*EE | O | O | S | O | S | O | F | OH |
| 5025a-l | Y*EE | O | O | S | O | S | O | OEt | OH |
| 5026a-l | Y*EE | S | O | S | O | S | O | OH | OH |
| 5027a-l | Y*EE | S | O | S | O | S | O | OMe | OH |
| 5028a-l | Y*EE | S | O | S | O | S | O | H | OH |
| 5029a-l | Y*EE | S | O | S | O | S | O | F | OH |
| 5030a-l | Y*EE | S | O | S | O | S | O | OEt | OH |
| 5031a-l | Y*EE | S | S | S | O | S | O | OH | OH |
| 5032a-l | Y*EE | S | S | S | O | S | O | OMe | OH |
| 5033a-l | Y*EE | S | S | S | O | S | O | H | OH |
| 5034a-l | Y*EE | S | S | S | O | S | O | F | OH |
| 5035a-l | Y*EE | S | S | S | O | S | O | OEt | OH |
| 5036a-l | Y*EE | O | O | O | S | O | S | OH | OH |
| 5037a-l | Y*EE | O | O | O | S | O | S | OMe | OF |
| 5038a-l | Y*EE | O | O | O | S | O | S | H | OH |
| 5039a-l | Y*EE | O | O | O | S | O | S | F | OH |
| 5040a-l | Y*EE | O | O | O | S | O | S | OEt | OH |
| 5041a-l | Y*EE | S | O | O | S | O | S | OH | OH |
| 5042a-l | Y*EE | S | O | O | S | O | S | OMe | OH |
| 5043a-l | Y*EE | S | O | O | S | O | S | H | OH |
| 5044a-l | Y*EE | S | O | O | S | O | S | F | OH |
| 5045a-l | Y*EE | S | O | O | S | O | S | OEt | OH |
| 5046a-l | Y*EE | S | S | O | S | O | S | OH | OH |
| 5047a-l | Y*EE | S | S | O | S | O | S | OMe | OH |
| 5048a-l | Y*EE | S | S | O | S | O | S | H | OH |
| 5049a-l | Y*EE | S | S | O | S | O | S | F | OH |
| 5050a-l | Y*EE | S | S | O | S | O | S | OEt | OH |
| 5051a-l | Y*EE | O | O | O | O | O | O | OH | OMe |
| 5052a-l | Y*EE | O | O | O | O | O | O | OMe | OMe |
| 5053a-l | Y*EE | O | O | O | O | O | O | H | OMe |
| 5054a-l | Y*EE | O | O | O | O | O | O | F | OMe |
| 5055a-l | Y*EE | O | O | O | O | O | O | OEt | OMe |
| 5056a-l | Y*EE | S | O | O | O | O | O | OH | OMe |
| 5057a-l | Y*EE | S | O | O | O | O | O | OMe | OMe |
| 5058a-l | Y*EE | S | O | O | O | O | O | H | OMe |
| 5059a-l | Y*EE | S | O | O | O | O | O | F | OMe |
| 5060a-l | Y*EE | S | O | O | O | O | O | OEt | OMe |
| 5061a-l | Y*EE | S | S | O | O | O | O | OH | OMe |
| 5062a-l | Y*EE | S | S | O | O | O | O | OMe | OMe |
| 5063a-l | Y*EE | S | S | O | O | O | O | H | OMe |
| 5064a-l | Y*EE | S | S | O | O | O | O | F | OMe |
| 5065a-l | Y*EE | S | S | O | O | O | O | OEt | OMe |
| 5066a-l | Y*EE | O | O | S | O | O | O | OH | OMe |
| 5067a-l | Y*EE | O | O | S | O | O | O | OMe | OMe |
| 5068a-l | Y*EE | O | O | S | O | O | O | H | OMe |
| 5069a-l | Y*EE | O | O | S | O | O | O | F | OMe |
| 5070a-l | Y*EE | O | O | S | O | O | O | OEt | OMe |
| 5071a-l | Y*EE | S | O | S | O | O | O | OH | OMe |
| 5072a-l | Y*EE | S | O | S | O | O | O | OMe | OMe |
| 5073a-l | Y*EE | S | O | S | O | O | O | H | OMe |
| 5074a-l | Y*EE | S | O | S | O | O | O | F | OMe |
| 5075a-l | Y*EE | S | O | S | O | O | O | OEt | OMe |
| 5076a-l | Y*EE | S | S | S | O | O | O | OH | OMe |
| 5077a-l | Y*EE | S | S | S | O | O | O | OMe | OMe |
| 5078a-l | Y*EE | S | S | S | O | O | O | H | OMe |
| 5079a-l | Y*EE | S | S | S | O | O | O | F | OMe |
| 5080a-l | Y*EE | S | S | S | O | O | O | OEt | OMe |
| 5081a-l | Y*EE | O | O | S | S | O | O | OH | OMe |
| 5082a-l | Y*EE | O | O | S | S | O | O | OMe | OMe |
| 5083a-l | Y*EE | O | O | S | S | O | O | H | OMe |
| 5084a-l | Y*EE | O | O | S | S | O | O | F | OMe |
| 5085a-l | Y*EE | O | O | S | S | O | O | OEt | OMe |
| 5086a-l | Y*EE | S | O | S | S | O | O | OH | OMe |
| 5087a-l | Y*EE | S | O | S | S | O | O | OMe | OMe |
| 5088a-l | Y*EE | S | O | S | S | O | O | H | OMe |
| 5089a-l | Y*EE | S | O | S | S | O | O | F | OMe |
| 5090a-l | Y*EE | S | O | S | S | O | O | OEt | OMe |
| 5091a-l | Y*EE | S | S | S | S | O | O | OH | OMe |
| 5092a-l | Y*EE | S | S | S | S | O | O | OMe | OMe |
| 5093a-l | Y*EE | S | S | S | S | O | O | H | OMe |
| 5094a-l | Y*EE | S | S | S | S | O | O | F | OMe |
| 5095a-l | Y*EE | S | S | S | S | O | O | OEt | OMe |
| 5096a-l | Y*EE | O | O | S | S | S | O | OH | OMe |
| 5097a-l | Y*EE | O | O | S | S | S | O | OMe | OMe |
| 5098a-l | Y*EE | O | O | S | S | S | O | H | OMe |
| 5099a-l | Y*EE | O | O | S | S | S | O | F | OMe |
| 5100a-l | Y*EE | O | O | S | S | S | O | OEt | OMe |
| 5101a-l | Y*EE | S | O | S | S | S | O | OH | OMe |
| 5102a-l | Y*EE | S | O | S | S | S | O | OMe | OMe |
| 5103a-l | Y*EE | S | O | S | S | S | O | H | OMe |
| 5104a-l | Y*EE | S | O | S | S | S | O | F | OMe |
| 5105a-l | Y*EE | S | O | S | S | S | O | OEt | OMe |
| 5106a-l | Y*EE | S | S | S | S | S | O | OH | OMe |
| 5107a-l | Y*EE | S | S | S | S | S | O | OMe | OMe |
| 5108a-l | Y*EE | S | S | S | S | S | O | H | OMe |
| 5109a-l | Y*EE | S | S | S | S | S | O | F | OMe |
| 5110a-l | Y*EE | S | S | S | S | S | O | OEt | OMe |
| 5111a-l | Y*EE | O | O | S | S | S | S | OH | OMe |
| 5112a-l | Y*EE | O | O | S | S | S | S | OMe | OMe |
| 5113a-l | Y*EE | O | O | S | S | S | S | H | OMe |
| 5114a-l | Y*EE | O | O | S | S | S | S | F | OMe |
| 5115a-l | Y*EE | O | O | S | S | S | S | OEt | OMe |
| 5116a-l | Y*EE | S | O | S | S | S | S | OH | OMe |
| 5117a-l | Y*EE | S | O | S | S | S | S | OMe | OMe |
| 5118a-l | Y*EE | S | O | S | S | S | S | H | OMe |
| 5119a-l | Y*EE | S | O | S | S | S | S | F | OMe |
| 5120a-l | Y*EE | S | O | S | S | S | S | OEt | OMe |
| 5121a-l | Y*EE | S | S | S | S | S | S | OH | OMe |
| 5122a-l | Y*EE | S | S | S | S | S | S | OMe | OMe |
| 5123a-l | Y*EE | S | S | S | S | S | S | H | OMe |
| 5124a-l | Y*EE | S | S | S | S | S | S | F | OMe |
| 5125a-l | Y*EE | S | S | S | S | S | S | OEt | OMe |
| 5126a-l | Y*EE | O | O | O | S | S | S | OH | OMe |
| 5127a-l | Y*EE | O | O | O | S | S | S | OMe | OMe |
| 5128a-l | Y*EE | O | O | O | S | S | S | H | OMe |
| 5129a-l | Y*EE | O | O | O | S | S | S | F | OMe |
| 5130a-l | Y*EE | O | O | O | S | S | S | OEt | OMe |
| 5131a-l | Y*EE | S | O | O | S | S | S | OH | OMe |
| 5132a-l | Y*EE | S | O | O | S | S | S | OMe | OMe |
| 5133a-l | Y*EE | S | O | O | S | S | S | H | OMe |
| 5134a-l | Y*EE | S | O | O | S | S | S | F | OMe |
| 5135a-l | Y*EE | S | O | O | S | S | S | OEt | OMe |
| 5136a-l | Y*EE | S | S | O | S | S | S | OH | OMe |
| 5137a-l | Y*EE | S | S | O | S | S | S | OMe | OMe |
| 5138a-l | Y*EE | S | S | O | S | S | S | H | OMe |
| 5139a-l | Y*EE | S | S | O | S | S | S | F | OMe |
| 5140a-l | Y*EE | S | S | O | S | S | S | OEt | OMe |
| 5141a-l | Y*EE | O | O | O | O | S | S | OH | OMe |
| 5142a-l | Y*EE | O | O | O | O | S | S | OMe | OMe |
| 5143a-l | Y*EE | O | O | O | O | S | S | H | OMe |
| 5144a-l | Y*EE | O | O | O | O | S | S | F | OMe |
| 5145a-l | Y*EE | O | O | O | O | S | S | OEt | OMe |
| 5146a-l | Y*EE | S | O | O | O | S | S | OH | OMe |
| 5147a-l | Y*EE | S | O | O | O | S | S | OMe | OMe |
| 5148a-l | Y*EE | S | O | O | O | S | S | H | OMe |
| 5149a-l | Y*EE | S | O | O | O | S | S | F | OMe |
| 5150a-l | Y*EE | S | O | O | O | S | S | OEt | OMe |
| 5151a-l | Y*EE | S | S | O | O | S | S | OH | OMe |
| 5152a-l | Y*EE | S | S | O | O | S | S | OMe | OMe |
| 5153a-l | Y*EE | S | S | O | O | S | S | H | OMe |
| 5154a-l | Y*EE | S | S | O | O | S | S | F | OMe |
| 5155a-l | Y*EE | S | S | O | O | S | S | OEt | OMe |
| 5156a-l | Y*EE | O | O | O | O | O | S | OH | OMe |
| 5157a-l | Y*EE | O | O | O | O | O | S | OMe | OMe |
| 5158a-l | Y*EE | O | O | O | O | O | S | H | OMe |
| 5159a-l | Y*EE | O | O | O | O | O | S | F | OMe |
| 5160a-l | Y*EE | O | O | O | O | O | S | OEt | OMe |
| 5161a-l | Y*EE | S | O | O | O | O | S | OH | OMe |
| 5162a-l | Y*EE | S | O | O | O | O | S | OMe | OMe |
| 5163a-l | Y*EE | S | O | O | O | O | S | H | OMe |
| 5164a-l | Y*EE | S | O | O | O | O | S | F | OMe |
| 5165a-l | Y*EE | S | O | O | O | O | S | OEt | OMe |
| 5166a-l | Y*EE | S | S | O | O | O | S | OH | OMe |
| 5167a-l | Y*EE | S | S | O | O | O | S | OMe | OMe |
| 5168a-l | Y*EE | S | S | O | O | O | S | H | OMe |
| 5169a-l | Y*EE | S | S | O | O | O | S | F | OMe |
| 5170a-l | Y*EE | S | S | O | O | O | S | OEt | OMe |
| 5171a-l | Y*EE | O | O | S | O | S | O | OH | OMe |
| 5172a-l | Y*EE | O | O | S | O | S | O | OMe | OMe |
| 5173a-l | Y*EE | O | O | S | O | S | O | H | OMe |
| 5174a-l | Y*EE | O | O | S | O | S | O | F | OMe |
| 5175a-l | Y*EE | O | O | S | O | S | O | OEt | OMe |
| 5176a-l | Y*EE | S | O | S | O | S | O | OH | OMe |
| 5177a-l | Y*EE | S | O | S | O | S | O | OMe | OMe |
| 5178a-l | Y*EE | S | O | S | O | S | O | H | OMe |
| 5179a-l | Y*EE | S | O | S | O | S | O | F | OMe |
| 5180a-l | Y*EE | S | O | S | O | S | O | OEt | OMe |
| 5181a-l | Y*EE | S | S | S | O | S | O | OH | OMe |
| 5182a-l | Y*EE | S | S | S | O | S | O | OMe | OMe |
| 5183a-l | Y*EE | S | S | S | O | S | O | H | OMe |
| 5184a-l | Y*EE | S | S | S | O | S | O | F | OMe |
| 5185a-l | Y*EE | S | S | S | O | S | O | OEt | OMe |
| 5186a-l | Y*EE | O | O | O | S | O | S | OH | OMe |
| 5187a-l | Y*EE | O | O | O | S | O | S | OMe | OMe |
| 5188a-l | Y*EE | O | O | O | S | O | S | H | OMe |
| 5189a-l | Y*EE | O | O | O | S | O | S | F | OMe |
| 5190a-l | Y*EE | O | O | O | S | O | S | OEt | OMe |
| 5191a-l | Y*EE | S | O | O | S | O | S | OH | OMe |
| 5192a-l | Y*EE | S | O | O | S | O | S | OMe | OMe |
| 5193a-l | Y*EE | S | O | O | S | O | S | H | OMe |
| 5194a-l | Y*EE | S | O | O | S | O | S | F | OMe |
| 5195a-l | Y*EE | S | O | O | S | O | S | OEt | OMe |
| 5196a-l | Y*EE | S | S | O | S | O | S | OH | OMe |
| 5197a-l | Y*EE | S | S | O | S | O | S | OMe | OMe |
| 5198a-l | Y*EE | S | S | O | S | O | S | H | OMe |
| 5199a-l | Y*EE | S | S | O | S | O | S | F | OMe |
| 5200a-l | Y*EE | S | S | O | S | O | S | OEt | OMe |

Y*NN (1001a-l to 4360a-l)
B is Adenine (A), Cytosine (C), Guanine (G), Uracil (U), Thymine (T), Hypoxanthine (I) or Purine (E); X is O or S and X′ is O or S; Z is O or S and Z′ is O or S and Z″ is O or S and Z′′′ is O or S; R′ is OH, OMe, H, F, or OEt and R″ is OH or OMe
Each N in Y*NN indicates nucleotide/nucleoside carrying A, C, G, U, T, I or E, where NN is a dinucleotide moiety of any combination of A, C, G, U, T, I and E. 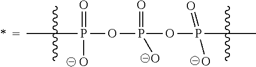 [SMILES: *P(=O)([O-])OP(=O)([O-])OP(*)(=O)[O-]] 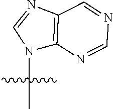 [SMILES: *n1cnc2cncnc21]
Purine (E) 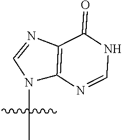 [SMILES: *n1cnc2c(=O)[nH]cnc21]
Hypoxanthine (I)

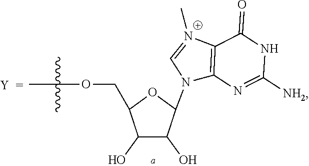

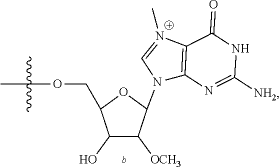 [SMILES: *OCC1OC(n2c[n+](C)c3c(=O)[nH]c(N)nc32)C(OC)C1O]

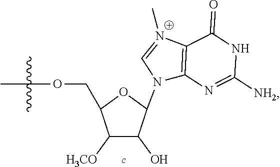 [SMILES: *OCC1OC(n2c[n+](C)c3c(=O)[nH]c(N)nc32)C(O)C1OC]

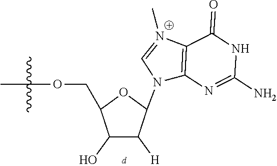 [SMILES: *OCC1OC(n2c[n+](C)c3c(=O)[nH]c(N)nc32)CC1O]

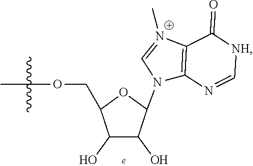 [SMILES: *OCC1OC(n2c[n+](C)c3c(=O)[nH]cnc32)C(O)C1O]

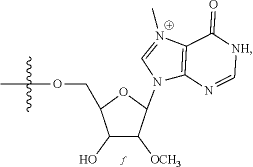 [SMILES: *OCC1OC(n2c[n+](C)c3c(=O)[nH]cnc32)C(OC)C1O]

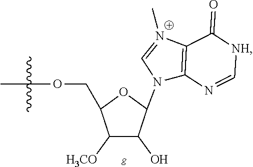 [SMILES: *OCC1OC(n2c[n+](C)c3c(=O)[nH]cnc32)C(O)C1OC]

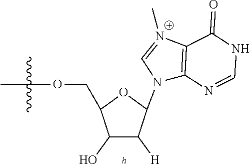 [SMILES: *OCC1OC(n2c[n+](C)c3c(=O)[nH]cnc32)CC1O]

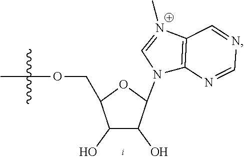 [SMILES: *OCC1OC(n2c[n+](C)c3cncnc32)C(O)C1O]

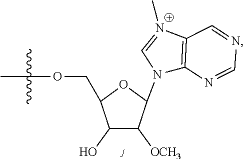 [SMILES: *OCC1OC(n2c[n+](C)c3cncnc32)C(OC)C1O]

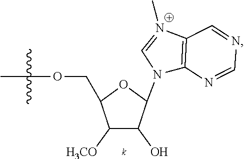 [SMILES: *OCC1OC(n2c[n+](C)c3cncnc32)C(O)C1OC]

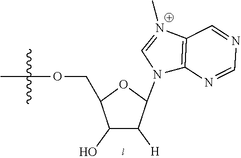 [SMILES: *OCC1OC(n2c[n+](C)c3cncnc32)CC1O]

In some embodiments, disclosed herein is a pharmaceutically acceptable salt or pharmaceutically acceptable solvate or a pharmaceutical composition comprising one or more mRNA(s) which is/are produced/manufactured from one or more sequence initiator compound selected from Table 3.

In another aspect, described herein is a complex comprising an IVT mRNA sequence initiator and a DNA template, wherein the IVT mRNA sequence initiator comprises a compound described herein, wherein (a) the DNA template comprises a promoter region comprising a transcriptional start site having a first nucleotide at nucleotide position +1, a second nucleotide at nucleotide position +2, and a third nucleotide at nucleotide position +3; and (b) wherein the IVT mRNA sequence initiator is hybridized to the DNA template at least at nucleotide positions +1, +2, and +3.

In another aspect, described herein is a complex comprising an IVT mRNA sequence initiator and a DNA template, wherein the IVT mRNA sequence initiator comprises a compound described herein, wherein (a) the DNA template comprises a promoter region comprising a transcriptional start site having a first nucleotide at nucleotide position +1 and a second nucleotide at nucleotide position +2; and (b) wherein the IVT mRNA sequence initiator is hybridized to the DNA template at least at nucleotide positions +1 and +2.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition can be a mixture of a sequence initiator compound described herein with one or more other chemical components (i.e. pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

The compositions described herein can be administered to the subject in a variety of ways, including parenterally, intravenously, intradermally, intramuscularly, colonically, rectally or intraperitoneally. In some embodiments, the sequence initiator compound described herein or a pharmaceutically acceptable salt thereof is administered by intraperitoneal injection, intramuscular injection, subcutaneous injection, or intravenous injection of the subject. In some embodiments, the pharmaceutical compositions can be administered parenterally, intravenously, intramuscularly or orally. The oral agents comprising a sequence initiator compound described herein can be in any suitable form for oral administration, such as liquid, tablets, capsules, or the like. The oral formulations can be further coated or treated to prevent or reduce dissolution in stomach. The compositions of the present disclosure can be administered to a subject using any suitable methods known in the art. Suitable formulations for use in the present disclosure and methods of delivery are generally well known in the art. For example, the sequence initiator compound described herein can be formulated as pharmaceutical compositions with a pharmaceutically acceptable diluent, carrier or excipient. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

Pharmaceutical formulations described herein can be administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the pharmaceutical formulation is in the form of a tablet. In other embodiments, pharmaceutical formulations containing a sequence initiator compound described herein are in the form of a capsule. In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a sequence initiator compound described herein can be formulated for use as an aerosol, a mist or a powder. For buccal or sublingual administration, the compositions may take the form of tablets, lozenges, or gels formulated in a conventional manner. In some embodiments, a sequence initiator compound described herein can be prepared as transdermal dosage forms. In some embodiments, a sequence initiator compound described herein can be formulated into a pharmaceutical composition suitable for intramuscular, subcutaneous, or intravenous injection. In some embodiments, a sequence initiator compound described herein can be administered topically and can be formulated into a variety of topically administrable compositions, such as solutions, suspensions, lotions, gels, pastes, medicated sticks, balms, creams or ointments. In some embodiments, a sequence initiator compound described herein can be formulated in rectal compositions such as enemas, rectal gels, rectal foams, rectal aerosols, suppositories, jelly suppositories, or retention enemas.

In one aspect, described herein is a pharmaceutical composition comprising an RNA molecule comprising the IVT mRNA sequence initiator disclosed herein and one or more of pharmaceutically acceptable excipients. In some embodiments, the pharmaceutical composition comprises lipid nanoparticles. In some embodiments, the RNA is encapsulated in an lipid nanoparticle.

### Transcription

In Eukaryotes, transcription of messenger RNAs (mRNAs) is done by RNA polymerase II. This is a complicated multi-subunit enzyme with complex regulation. To carry out large scale transcription in vitro, researches commonly use single subunit phage polymerases derived from T7, T3, SP6, K1-5, K1E, K1F or K11 bacteriophages. This family of polymerases has simple, minimal promoter sequences of ˜17 nucleotides which require no accessory proteins and have minimal constraints of the initiating nucleotide sequence. While this application focuses on T7 RNA Polymerase (T7 RNAP), one skilled in the art would understand that this disclosure could be practiced with other RNA polymerases.

T7 RNAP exists in at least two protein states. The first is referred to as the “abortive complex” and is associated with transcriptional initiation. The second is a very processive conformation called the “elongation complex”. In vitro transcription can be broken into six steps: 1) binding of the RNA polymerase to the promoter sequence, 2) initiation of transcription, 3) non-processive elongation termed abortive transcription during which the polymerase frequently releases the DNA template and short abortive transcripts 4) conversion of the open complex to the closed complex, 5) processive elongation and 6) transcriptional termination. A significant amount of RNA produced during transcription consists of short abortive fragments of ˜2-8 nucleotides in length (Biochemistry 19:3245-3253 (1980); Nucleic Acids Res. 9:31-45 (1981); Nucleic Acids Res. 15:8783-8798 (1987); Biochemistry 27:3966-3974 (1988)). After synthesis of about 10-14 bases, RNA polymerases escape from abortive cycling, at the same time losing sequence-specific contacts with the promoter DNA, and forming a processive elongation complex, in which the RNA chain is extended in a sequence-independent manner (J. Mol. Biol. 183:165-177 (1985); Proc. Natl. Acad. Sci. U.S.A. 83:3614-3618 (1986); Mol. Cell Biol. 7:3371-3379 (1987)).

The consensus sequence for the most active Class III T7 promoters encompasses 17 bp of sequence upstream, and 6 bp downstream, of the transcription start site (Cell 16:815-25. (1979)). The position of the first transcribed nucleotide is commonly referred to as the +1 transcript nucleotide of the RNA, the second transcribed nucleotide as +2 transcript nucleotide and so on (Table 2). During transcription, the two strands are melted to form a transcription bubble and the bottom strand of the duplex (shown 3′ to 5′ in Table 4) is the template for transcription. For transcript nucleotides +3 and beyond, the template strand defines the identity of the transcribed nucleotides primarily through Watson-Crick base pairing interactions. Here the nucleotide encoding the first RNA transcript nucleotide is defined as the +1 nucleotide of the template. In the example shown in Table 4, the +1 transcript nucleotide is G and the +1 template nucleotide is C. Likewise the +4 transcript nucleotide is A and the +4 template nucleotide is T.

TABLE 4

| | |
|---|---|
| Position in transcript Transcript sequence Promoter top strand Promoter bottom strand Position in template | 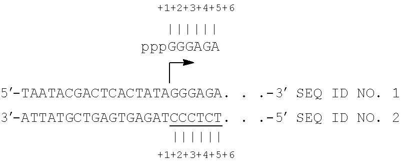 |

Unlike DNA polymerases, T7 RNAP initiates RNA synthesis in the absence of a primer. The first step in initiation is called de novo RNA synthesis, in which RNA polymerase recognizes a specific sequence on the DNA template, selects the first pair of nucleotide triphosphates complementary to template residues at positions +1 and +2, and catalyzes the formation of a phosphodiester bond to form a dinucleotide. The initiating nucleotides have lower affinities for the polymerase than those used during elongation. The Kd value is 2 mM for the first initiating NTP and 80 μM for the second, whereas the Kd is approximately 5 μM for NTPs during elongation (J. Mol. Biol. (2007) 370, 256-268). It has been found that de novo synthesis is the rate-limiting step during transcription. T7 RNAP exhibits a strong bias for GTP as the initiating nucleotide (J. Biol. Chem. 248: 2235-2244 (1973)). Among the 17 T7 promoters in the genome, 15 initiate with GTP (and 13 with pppGpG), whereas there is no obvious NTP preference during transcription elongation (J. Mol. Biol. 370:256-268 (2007)). T7 RNA polymerase initiates poorly on promoters encoding A at position +1; transcription instead initiates predominantly with an encoded G at position +2 (J Biol. Chem. 278:2819-2823 (2003)).

During de novo RNA synthesis, binding of the initiating nucleotides is achieved primarily by the free energy created from base stacking, specific interactions between the polymerase residues, the guanine moieties of the initiating nucleotides and base complementarity interactions (J. Mol. Biol. 370:256-268 (2007)).

It is known that T7 RNAP can also initiate with short oligonucleotide primers. For example, it is known that 13 promoters in the T7 genome initiate with pppGpG (J. Mol. Biol. 370:256-268 (2007)). Several groups showed that T7 RNAP can initiate from dinucleotide primers (Biochemistry 24:5716-5723 (1985)). Axelrod et al. showed that an uncapped GpA dinucleotide could initiate from +1 and +2 template nucleotides that were 2′-deoxycytidine and 2′-deoxythymidine, respectively (“CT” template) Their reaction conditions were 200 micromolar (μM) dimer and 100 μM ATP, CTP, GTP and UTP. Their reaction mixture also contained 100 μM 3′ dATP, 3′ dCTP 3′ dUTP or 50 μM 3′ dGTP. They observe only GpA initiated RNAs and not a mixture of GpA initiated RNAs and 5′ triphosphate RNAs from GTP initiation. This is likely due to the reaction conditions employed. 100 μM GTP is well below the 2 mM Kd of T7 polymerase for the first initiating guanosine (J. Mol. Biol. (2007) 370, 256-268). Since GTP competes for initiation with the initiating oligonucleotide, using a low GTP concentration favors GpA initiation but results in low transcription yield (maximum calculated yield estimated to be <150 μg/mL of reaction). When initiating transcription on “CT” template with ApG, CpG, UpG or GpG, they observed formation of RNA transcripts with an additional untemplated 5′ nucleotide (A, C, U or G, respectively).

The methods and compositions provided herein for preparation of 5′-capped RNA include, but are not limited to, mRNA, small nuclear RNA (snRNA), small nucleolar RNA (snoRNA), small cajal body-specific RNA (scaRNA). These methods involve the use of a mRNA sequence initiator, for example, Cap containing oligonucleotide primers, along with nucleoside 5′-triphosphates (NTPs) and RNA polymerase for DNA-templated and promoter controlled synthesis of RNA. In certain aspects, the methods use an initiating capped oligonucleotide primer that provides utility in RNA synthesis, in particular synthesis of capped mRNAs. The exemplary initiating oligonucleotide primer has a structure that resembles Cap 0, Cap 1, Cap 2 or TMG-Cap of natural RNA molecules, which include 2′-O-methylated nucleoside units at, penultimate Cap 1 and next to penultimate Cap 2 5′-positions of RNA. The natural Cap 0 structure does not have 2′-O-methylated nucleoside units.

The methods and compositions for preparation of RNA including, but not limited to, mRNA, snRNA, snoRNA, scaRNA, transfer RNA (tRNA), ribosomal RNA (rRNA), and transfer-messenger RNA (tmRNA) that carry modifications at or near 5′-end of the molecule. These methods involve the use of initiators, e.g. initiating oligonucleotide primers with or without Cap, nucleoside 5′-triphosphates (NTPs) and RNA polymerase for DNA-templated and promoter controlled synthesis of RNA. In certain aspects, the methods use a modified initiating oligonucleotide primer carrying structural modifications that provide utility in RNA synthesis; in particular synthesis of 5′-modified RNAs.

An exemplary initiating capped oligonucleotide primer has an open 3′-OH group that allows for initiation of RNA polymerase mediated synthesis of RNA on a DNA template by adding nucleotide units to the 3′-end of the primer. The initiating capped oligonucleotide primer is substantially complementary to template DNA sequence at the transcription initiation site (i.e., the initiation site is located closer to 3′-terminus of a promoter sequence and may overlap with promoter sequence). In certain embodiments, the initiating capped oligonucleotide primer directs synthesis of RNA predominantly in one direction (“forward”) starting from the 3′-end of the primer. In certain aspects and embodiments, the initiating capped oligonucleotide primer outcompetes any nucleoside 5′-triphosphate for initiation of RNA synthesis, thereby maximizing the production of the RNA that starts with initiating capped oligonucleotide primer and minimizing a production of RNA that starts with 5′-triphosphate-nucleoside (typically GTP).

The manufacture of mRNA by in vitro transcription utilizes highly active phage RNA polymerases (T3, T7, SP6 and others). RNA polymerase works under control of specific promoter which is incorporated in DNA plasmid construct in front of a template nucleotide sequence. Transcription process usually starts with purine nucleoside 5′-triphosphate (typically GTP) and continues until the RNA polymerase encounters a terminating sequence or completes the DNA template.

As discussed above, mCAP dinucleotide analogs, ^{7m}G(5′)ppp(5′) N, that contain Cap 0 have been used for initiation in vitro transcription (e.g., RNA 1: 957-967 (1995)). The capped RNA molecules produced using these dinucleotide analogs contain Cap 0. However only about 50% of synthesized capped RNA molecules have the correct “forward” orientation of Cap 0. To convert RNA with Cap 0 to RNA with Cap 1 an additional enzymatic reaction must be preformed using (nucleoside-2′-O) methyltransferase. However this conversion may be not quantitative; it is not easy to control and it is difficult to separate the remaining Cap 0 RNAs from Cap 1 RNAs. In addition, the competition from NTPs (specifically GTP) for initiation transcription further reduces the quantity of active capped RNA molecules produced.

In addition, modified dinucleotide analogs, such as ^{7m}G_{3′Ome}(5′)ppp(5′) N and other related ARCA analogs, that carry modified ^{7m}G residue with blocked 3′ and/or 2′ position on ribose, have been used for initiation of in vitro transcription (e.g., RNA 7:1486-1495 (2001)). These ARCA cap analogs direct RNA synthesis only in the “forward” orientation and therefore produce a RNA molecule with (natural) Cap 0 on the 5′-terminus (having 2′ and/or 3′ modifications to ^{7m}G residue). Such RNAs are more active in translation systems compared to RNAs prepared using standard dinucleotide analogs, ^{7m}G(5′)ppp(5′) N. To convert RNA with ARCA Cap 0 to RNA with ARCA Cap 1 an additional enzymatic reaction must be performed with (nucleoside-2′-O) methyltransferase similar to that required for the dinucleotide analogs previously discussed. This method has the same disadvantages as that elaborated for the mCAP dinucleotide analogs; the conversion of RNA with Cap 0 to RNA with ARCA Cap 1 may be not quantitative; the reaction is not easy to control, it is difficult to separate remaining Cap 0 RNAs from Cap 1 RNAs and competition from NTPs (specifically GTP) for initiation of transcription further reduces the quantity of active capped RNA molecules produced.

Short oligonucleotide primers (2 to 6-mer) with 3′-terminal guanosine residue have been used for initiation of in vitro transcription (Pitulle, C. et al., Gene, 112:101-105 (1992)). These oligonucleotide primers contained modified and unmodified ribonucleoside residues (e.g., modified ribonucleoside residues included 2′-O-methylated nucleoside residues and 2′-deoxyribonucleoside residues). The shorter oligonucleotide primers (dimers to tetramer) substantially out-compete GTP for initiation of transcription while longer primers (pentamer to hexamer) are much less efficient in initiation of transcription compared to GTP. It may be because these longer primers (as they are designed) have a low percent of complementarity with DNA template at initiation site. In contrast, dimer, AG (as designed), was complementary to the DNA template at initiation site. The RNA molecules produced using oligonucleotide primers, discussed in this section, had internal 2′-O-methylated nucleoside but did not contain 5′-Cap 0, Cap1, Cap 2 or TMG-cap. To convert RNA without cap structure to RNA with Cap 1, Cap 2 or TMG-cap structure an additional enzymatic reactions using capping enzymes would have to be performed. However such conversion has the same disadvantages as those stated above.

Other short RNA oligonucleotides containing cap structures and internal 2′-O-methylated nucleoside residues have been chemically prepared (Ohkubo et al., Org. Letters 15:4386-4389 (2013)). These short capped oligonucleotides were ligated with a “decapitated” (without 5′-cap structure) fragment of long RNA using T4 DNA ligase and a complementary DNA splinter oligonucleotide. The final RNA synthesized using this chemical-enzymatic method had both internal 2′-O-methylated nucleoside residues and 5′-TMG-cap structure. However only short capped RNAs (<200-mer) were prepared using this ligation approach. Moreover, the yields were low (15-30%). It is not easy to control and optimize the T4 DNA ligation reaction and it requires a laborious separation process using PolyAcrylamide Gel Electrophoresis and isolation of capped RNAs from remaining uncapped RNAs. Separation of long (500-10000 bases) capped mRNAs from remaining uncapped mRNAs by PAGE method is not feasible.

Finally, 5′-modified nucleoside or 5′-modified mononucleotide or 5′-modified dinucleotide, typically a derivative of guanosine, have been used for initiating in vitro transcription of RNA (Gene, 112:101-105 (1992) and Bioconjug. Chem., 10371-378 (1999)). These initiator nucleosides and nucleotides may carry labels or affinity groups (e.g. biotin) and, when incorporated on the 5′-end of RNA, would allow for easy detection, isolation and purification of synthesized RNA. This 5′-labeled or tagged RNAs may be necessary for some applications. However this strategy was not used for the preparation of mRNA with Cap 0, Cap 1, Cap 2 or TMG-cap structures.

In certain aspects of the present disclosure, compositions of the capped mRNA sequence initiators as described herein are provided. In related aspects, are methods in which RNA is synthesized using capped mRNA sequence initiators described herein.

mRNA Sequence Initiators

Provided herein are mRNA sequence initiators that may be used for RNA synthesis. mRNA sequence initiators may have a hybridization sequence which may be complementary to a sequence on DNA template at initiation site. In some embodiments, the length of the hybridization sequence of the initiators for use in the methods and compositions provided herein depends on several factors including the identity of template nucleotide sequence and the temperature at which this initiator is hybridized to DNA template or used during in vitro transcription. Determination of the desired length of a specific nucleotide sequence of a capped initiator for use in transcription can be easily determined by a person of ordinary skill in the art or by routine experimentation. For example, the length of a nucleic acid or oligonucleotide may be determined based on a desired hybridization specificity or selectivity.

mRNA sequence initiators may be capped oligonucleotide primers. In some embodiments, the nucleotide length of initiating capped oligonucleotide primer (including the inverted 5′-5′ Cap nucleotide) is between 3 to about 9, in some embodiments the nucleotide length of initiating capped oligonucleotide primer (including Cap) is between 3 to about 7, in some embodiments the nucleotide length of initiating capped oligonucleotide primer (including Cap) is between 3 to about 5, and in some embodiments the nucleotide length of initiating capped oligonucleotide primer (including Cap) is about 3. The length of hybridization sequence within an initiator, e.g., an initiating capped oligonucleotide primer may be equal to or shorter than the total length of the initiating capped oligonucleotide primer.

The presence of hybridization sequence forces an initiator, e.g. an initiating capped oligonucleotide primer, to predominantly align with complementary sequence of the DNA template at the initiation site in only the desired orientation (i.e., the “forward” orientation). In some embodiments, in the forward orientation, the RNA transcript begins with the inverted guanosine residue. The dominance of the forward orientation of primer alignment on DNA template over incorrect “reverse” orientation is maintained by the thermodynamics of the hybridization complex. The latter may be determined by the length of the hybridization sequence of initiating capped oligonucleotide primer and the identity of bases involved in hybridization with DNA template. Hybridization in the desired forward orientation may also depend on the temperature and reaction conditions at which DNA template and initiating capped oligonucleotide primer are hybridized or used during in vitro transcription.

An exemplary initiator, e.g., an initiating capped oligonucleotide primer of the present disclosure enhances efficacy of initiation of transcription compared to efficacy of initiation with standard GTP, ATP, CTP or UTP. In some embodiments, initiation of transcription is considered enhanced when synthesis of RNA starts predominantly from initiating capped oligonucleotide primer and not from any NTP in transcription mixture. The enhanced efficiency of initiation of transcription results in a higher yield of RNA transcript. The enhanced efficiency of initiation of transcription may be increased to about 10%, about 20%, about 40%, about 60%, about 80%, about 90%, about 100%, about 150%, about 200% or about 500% over synthesis of RNA with conventional methods without initiating capped primer. In certain embodiments, a capped mRNA sequence initiator, e.g. an initiating capped oligonucleotide primer, out-competes any NTP (including GTP) for initiation of transcription. One of ordinary skill in the art is able to readily determine the level of substrate activity and efficacy of initiating capped oligonucleotide primers. One example of a method of determining substrate efficacy is illustrated in Example 13). In certain embodiments, initiation takes place from a capped oligonucleotide primer rather than an NTP, which results in a higher level of capping of the transcribed mRNA.

In some aspects, methods are provided in which RNA is synthesized utilizing an initiating capped oligonucleotide primer that has substitutions or modifications. In some aspects, the substitutions and modifications of an initiating capped oligonucleotide primer do not substantially impair the synthesis of RNA. Routine test syntheses can be preformed to determine if desirable synthesis results can be obtained with the modified initiating capped oligonucleotide primers. Those skilled in the art can perform such routine experimentation to determine if desirable results can be obtained. The substitution or modification of initiating capped oligonucleotide primer include for example, one or more modified nucleoside bases, one or more modified sugars, one or more modified internucleotide linkage and/or one or more modified triphosphate bridges.

An initiator, for example, a modified initiating capped oligonucleotide primer, which may include one or more modification groups of the methods and compositions provided herein, can be elongated by RNA polymerase on DNA template by incorporation of NTP onto open 3′-OH group. An initiating capped oligonucleotide primer may include natural RNA and DNA nucleosides, modified nucleosides or nucleoside analogs. The initiating capped oligonucleotide primer may contain natural internucleotide phosphodiester linkages or modifications thereof, or combination thereof.

In one embodiment the modification group may be a thermally labile group which dissociates from a modified initiating capped oligonucleotide primer at an increasing rate as the temperature of the enzyme reaction medium is raised. Examples of thermally labile groups for oligonucleotides and NTPs are described in Nucleic Acids Res., 36:e131 (2008), Collect. Symp. Ser., 10:259-263 (2008) and Analytical Chemistry, 81:4955-4962 (2009).

In some aspects, methods are provided in which RNA is synthesized where at least one or more NTP is added to a transcription reaction may have a modification as disclosed herein. In some aspects, the modification of the at least one NTP does not substantially impair RNA polymerase mediated synthesis of RNA. The modification of NTP may include for example, one or more modified nucleoside bases, one or more modified sugars, one or more modified 5′-triphosphate. The modified NTP may incorporate onto the 3′-end of an initiating capped oligonucleotide primer and it does not block transcription and supports further elongation of the primer.

In another embodiment, the modification group of an initiating capped oligonucleotide primer may be a detectable label or detectable marker. Thus, following transcription, the target RNA, containing the detectable label or marker, can be identified by size, mass, color and/or affinity capture. In some embodiments, the detectable label or marker is a fluorescent dye; and the affinity capture label is biotin. In certain embodiments, one or more components of a transcription reaction (initiating capped oligonucleotide primer and/or NTPs) may be labeled with a detectable label or marker. Thus, following transcription, the RNA molecule can be identified, for example, by size, mass, affinity capture or color. In some embodiments, the detectable label is a fluorescent dye; and the affinity capture label is biotin.

In some aspects, an IVT mRNA sequence initiator comprises a phosphorothioate group. In some embodiments, an IVT mRNA sequence initiator comprises a chiral center. In some embodiments, the chiral center comprises a phosphorous center. A composition comprising a compound of the disclosure may comprise a racemic mixture (e.g., 50:50) of the R diastereomer and the S diastereomer of the compound. In some embodiments, a stereochemical purity of the IVT mRNA sequence initiator in the composition is at least 60%. For example, a composition may comprise 60% of the R diastereomer of the compound and 40% of the S diastereomer of the compound. In another example, a composition may comprise 60% of the S diastereomer of the compound and 40% of the R diastereomer of the compound. In some embodiments, a stereochemical purity of the IVT mRNA sequence initiator in the composition is at least 70%. In some embodiments, a stereochemical purity of the IVT mRNA sequence initiator in the composition is at least 80%. In some embodiments, a stereochemical purity of the IVT mRNA sequence initiator in the composition is at least 90%. In some embodiments, a stereochemical purity of the IVT mRNA sequence initiator in the composition is at least 92%. In some embodiments, a stereochemical purity of the IVT mRNA sequence initiator in the composition is at least 95%. In some embodiments, a stereochemical purity of the IVT mRNA sequence initiator in the composition is at least 96%. In some embodiments, a stereochemical purity of the IVT mRNA sequence initiator in the composition is at least 97%. In some embodiments, a stereochemical purity of the IVT mRNA sequence initiator in the composition is at least 98%. In some embodiments, a stereochemical purity of the IVT mRNA sequence initiator in the composition is at least 99%. Standard chemical and enzymatic synthesis methods may be utilized to synthesize mRNA sequence initiators of the present disclosure and are disclosed herein in the Examples section.

In some embodiments, the IVT mRNA sequence initiator increases mRNA potency in comparison to a commercially available 5′ end initiator (e.g., 1002a).

In some embodiments, the IVT mRNA sequence initiator improves mRNA manufacturability in comparison to a commercially available 5′ end initiator (e.g., 1002a).

In some embodiments, the IVT mRNA sequence initiator improved efficiency of incorporation within an mRNA during RNA synthesis, in comparison to a commercially available 5′ end initiator (e.g., 1002a).

In some embodiments, the IVT mRNA sequence initiator decreases the percentage of double stranged (ds) RNA yielded during RNA synthesis in comparison to a commercially available 5′ end initiator (e.g., 1002a).

In some embodiments, the IVT mRNA sequence initiator has increased affinity for protein biogenesis factor in comparison to a commercially available 5′ end initiator (e.g., 1002a). For example, the IVT mRNA sequence initiator may improve ribosome recruitment in comparison to a commercially available initiator.

In some embodiments, mRNA having the IVT mRNA sequence initiator motif increases the frequency of interpretation by a protein synthesis instrument in comparison to a mRNA having a commercially available 5′ end initiator (e.g., 1002a).

In some embodiments, mRNA having the IVT mRNA sequence initiator motif improves immunogenicity in a cell in comparison to mRNA having a commercially available 5′ end initiator (e.g., 1002a).

In some embodiments, mRNA having the IVT mRNA sequence initiator motif has increased stability in comparison to a mRNA having a commercially available 5′ end initiator (e.g., 1002a). In some embodiments, increased stability comprises increased resistance to cleavage by enzymes that target a triphosphate bridge.

In some embodiments, mRNA having the IVT mRNA sequence initiator increases percent editing efficiency in comparison to a mRNA having a commercially available 5′ end initiator (e.g., 1002a). In some embodiments, mRNA having the IVT mRNA sequence initiator does not affect percent editing efficiency in comparison to a mRNA having a commercially available 5′ end initiator (e.g., 1002a).

### Kits

Kits including mRNA sequence initiators for performing transcription are also contemplated. For example, kits may contain all transcription reagents for synthesis of common RNAs (e.g., FLuc mRNA). More specifically, a kit may contain: a mRNA sequence initiator; a container marked for transcription; instructions for performing RNA synthesis; and one or more reagents selected from the group consisting of one or more modified or unmodified initiating capped oligonucleotide primers, one or more unmodified NTPs, one or more modified NTPs (e.g., pseudouridine 5′-triphosphate), an RNA polymerase, other enzymes, a reaction buffer, magnesium and a DNA template.

mRNA sequence initiators of the present disclosure have a significant advantage over current methods and compositions involving use of various initiating nucleosides, nucleotides and oligonucleotides or use of polyphosphate dinucleotide derivatives containing Cap 0 structure, such as mCAP and ARCA. A mRNA sequence initiator, e.g., an initiating capped oligonucleotide primer may be compatible with existing transcription systems and reagents and no additional enzymes or reagents are required. In addition, the use of initiating capped oligonucleotide primers makes several non-enzymatic and enzymatic steps (such as capping and 2′-O-methylation) unnecessary thus reducing complexity of the process and a cost of RNA synthesis.

While the exemplary methods described herein relate to T7 RNA polymerase mediated transcription reaction, a number of other RNA polymerases known in the art for use in transcription reactions may be utilized with the compositions and methods of the present disclosure. Other enzymes, including natural or mutated variants that may be utilized include, for example, SP6 and T3 RNA polymerases and RNA polymerases from other sources including thermostable RNA polymerases.

Some nucleic acid replication and amplification methods may include transcription as a part of the process. Among these methods are: transcription mediated amplification (TMA) and nucleic acid sequence-based amplification (NASBA), DNA and RNA sequencing and other nucleic acid extension reactions known in the art. The skilled artisan will understand that other methods may be used either in place of, or together with, transcription methods, including variants of transcription reactions developed in the future.

### Therapeutic Uses

In one aspect, described herein is a method for reducing the risk of coronary disease in a subject in need thereof, comprising administering to the subject an effective amount of a pharmaceutical composition described herein.

In one aspect, described herein is the production of mRNAs containing a capped mRNA sequence initiator, e.g., an initiation capped oligonucleotide primer for use as therapeutic agents in a pharmaceutical composition, the introduction of RNAs containing the capped intiator into cells to treat a medical condition of the cells or the introduction of RNAs containing the capped initiator into cells that utilize those RNAs to produce proteins that may have a therapeutic affect on the host cells.

One method for treating a condition utilizing an RNA containing capped initiator comprises the step of administering the RNA containing the capped initiator described herein or a composition comprising such RNA to a subject having, or suspected of having a condition whose symptoms/symptomologies may be reduced in severity or eliminated.

An RNA containing a capped initiator as described herein, when formulated in a pharmaceutically acceptable carrier and/or pharmaceutically acceptable salt at a concentration of 4 mg/ml or less, is effective to produce a reduction of the symptoms and/or symptomologies by at least 20%, 30%, 40%, 50%, 60%, 70%, 80%, 90% or greater than an untreated individual with the pharmaceutically acceptable carrier alone.

Pharmaceutical compositions may be formulated for administration by injection or other appropriate routes known to those skilled in the art for treating a particular condition. An injectable composition for parenteral administration typically contains the active compound in a suitable solution and/or pharmaceutical carrier such as sterile physiological saline. The composition may also be formulated as a suspension in a lipid or phospholipid, in a liposomal suspension, or in an aqueous emulsion.

Methods for preparing a variety of compositions and/or formulations are known to those skilled in the art see Remington's Pharmaceutical Sciences (19^{th} Ed., Williams & Wilkins, 1995). The composition to be administered will contain a quantity of the selected compound in a pharmaceutically safe and effective amount for increasing expression of the desired protein in the target cells or tissue.

In some embodiments, the pharmaceutical composition contains at least 0.1% (w/v) of the compound, as described above, in some embodiments, the pharmaceutical composition contains greater than 0.1%, in some embodiments, the pharmaceutical composition contains up to about 10%, in some embodiments, the pharmaceutical composition contains up to about 5%, and in some embodiments, the pharmaceutical composition contains up to about 1% (w/v) of the compound. Choice of a suitable concentration depends on factors such as the desired dose, frequency and method of delivery of the active agent.

For treatment of a subject, such as a mammal or a human, dosages are determined based on factors such as the weight and overall health of the subject, the condition treated, severity of symptoms, etc. Dosages and concentrations are determined to produce the desired benefit while avoiding any undesirable side effects. Typical dosages of the subject compounds are in the range of about 0.0005 to 500 mg/day for a human patient, and ranging in some embodiments between about 1-100 mg/day. For example, higher dose regimens include e.g. 50-100, 75-100, or 50-75 mg/day, and lower dose regimens include e.g. 1-50, 25-50, or 1-25 mg/day.

### RNA Modification

Provided herein are RNA molecules modified with initiators, e.g. initiating capped oligonucleotide primers described herein suitable for targeted ex vivo and in vivo delivery systems. A modified RNA molecule may comprise two or more linked ribonucleic acid subunits. Non-limiting exemplary modified RNAs include CRISPR guide RNA, short interfering RNA (siRNA), microRNA (miRNA), short hairpin RNA (shRNA), small nuclear RNA (snRNA), messenger RNA (mRNA), precursor mRNA (pre-mRNA), antisense RNA (asRNA), and heteronuclear RNA (hnRNA). Modified RNAs as described herein encompass both the RNA sequence and any structural embodiment thereof, e.g. single stranded, double stranded, triple stranded, circular, helical, hairpin, stem loop, bulge, etc. A modified RNA may comprise a length of at least about 3, 4, 5, 6, 7, 8, 9, 10, 20, 30, 40, 50, 100, 200, 300, 400, or 500 bases. A modified RNA may comprise a length of at least about 1 kilobase (kb), 2 kb, 3 kb, 4 kb, 5 kb, 10 kb, 20 kb, 50 kb, or more. In some embodiments, the modified RNA is a CRISPR guide RNA (gRNA). A gRNA may be a single guide RNA or a dual guide RNA. In some embodiments, the modified RNA is a mRNA. In some embodiments, a mRNA can be isolated from a cell or a tissue. In some embodiments, a mRNA can be transcribed from a DNA. In some embodiments, a mRNA can be chemically synthesized.

In certain embodiments, modified RNA molecules provided herein are resistant to degradation by RNases or other exonucleases. In certain embodiments, modified RNA molecules provided herein are stabilized to prevent degradation by endonucleases. In some embodiments, modified RNA molecules provided herein are suitable for in vivo delivery and induces less cellular immune receptor activation (e.g. TLR, RIG-I) as compared to unmodified RNA. RNA modifications as described in Diebold (2008) Adv Drug Deliv Rev. April 29; 60(7):813-23) and Sorrentino (1998) Cell Mol Life Sci. August; 54(8):785-94, the entirety of both are incorporated herein by reference.

The nucleotides as described herein can be synthetic or chemically modified. For example, guide RNAs provided herein can be synthetic or chemically modified guide RNAs. The nucleotides in the guide RNA that are modified may be those corresponding to one or more nucleotides in the binding region of the guide RNA with Cas9 and/or the nucleotides in the binding region of the guide RNA with the target DNA. Remaining unmodified nucleotides of the guide RNA may be those required to be identified for minimal binding of Cas9 to the 2′-OH location on the bases. In some embodiments, the nucleotides may be modified at the 2′ position of the sugar moiety of the nucleotide. In some embodiments, the 2′-OH group of the sugar moiety is replaced by a group selected from H, OR, R, halo, SH, SR, H2, NHR, N(R)2 or CN, wherein R is C1-C6 alkyl, alkenyl or alkynyl and halo is F, CI, Br or I. Other modifications may include, inverted (deoxy) abasics, amino, fluoro, chloro, bromo, CN, CF, methoxy, imidazole, carboxylate, thioate, C1 to C10 lower alkyl, substituted lower alkyl, alkaryl or aralkyl, heterozycloalkyl; heterozycloalkaryl; aminoalkylamino; polyalkylamino or substituted silyl. Methods for making RNAs with specific sequences and modifications are known by those skilled in the art, for example, in Dellinger et al. (2011), J. Am. Chem. Soc, 133, 11540; U.S. Pat. No. 8,202,983; Kumar et al., (2007), J. Am. Chem. Soc, 129, 6859-64; WO2013176844, the entirety of which are incorporated herein by reference.

In some embodiments, polynucleotides or oligonucleotides as provided herein may be synthetic. For example, mRNAs maybe chemically synthesized mRNAs. Synthetic RNA production yield is based on sequences and modifications. 2′ —O— methyl modifications have been shown to increase coupling efficacy or efficiency during RNA synthesis and therefore increase yield of chemically synthesized RNA. Furthermore, nucleotides may be modified by phosphorothioates. Phosphothioate (phosphorothioate)(PS) bonds substitute a sulphur atom for a non-bridging oxygen in the phosphate backbone of an oligonucleotide. Accordingly, exemplary nucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo configuration (a diastereomer of LNA), 2′-amino-LNA having a 2′-amino functionalization, and 2′-amino-a-LNA having a 2′-amino functionalization) or hybrids thereof.

### Conjugates for Targeted Delivery

Provided herein are conjugates suitable for targeted delivery of agents, such as mRNA, guide RNA, miRNA, siRNA, DNA, peptides, or other micro or macro molecules. A conjugate can contain one or more aptamers, ligands, or moieties for targeted delivery ex vivo or in vivo. In some embodiments, a conjugate comprises a targeting moiety (or ligand), a linker, and an active agent (or payload) that is connected to the targeting moiety. An active agent can be a therapeutic agent, a prophylactic agent, or a diagnostic/prognostic agent. An active agent may have a capability of manipulating a physiological function (e.g., gene expression) in a subject. An active agent maybe a guide RNA, a mRNA, a miRNA, a siRNA, a DNA, or a peptide. The active agent may be connected with the targeting moiety via a linker, via a non-covalent linkage, via nucleobase paring, or any combination thereof. In some embodiments, the conjugate may be a conjugate between a single active agent and a single targeting moiety with the formula: X—Y—Z, wherein X is the targeting moiety; Y is a linker; and Z is the guide RNA. In certain embodiments, one targeting ligand can be conjugated to two or more active agents, wherein the conjugate has the formula: X—(Y—Z)n. For example, the conjugate may comprise a guide RNA and a mRNA. In certain embodiments, one active agent can be linked to two or more targeting ligands wherein the conjugate has the formula: (X—Y)n-Z. In other embodiments, one or more targeting moieties may be connected to one or more active pay loads wherein the conjugate formula may be (X—Y—Z)n. In various combinations, the formula of the conjugates maybe, for example, X—Y—Z—Y—X, (X—Y—Z)n-Y—Z, or X—Y—(X—Y—Z)n, wherein X is a targeting moiety; Y is a linker; Z is an active agent, e.g. a guide RNA. The number of each moiety in the conjugate may vary dependent on types of agents, sizes of the conjugate, delivery targets, particles used to packaging the conjugate, other active agents (e.g., immunologic adjuvants) and routes of administration. Each occurrence of X, Y, and Z can be the same or different, e.g. the conjugate can contain more than one type of targeting moiety, more than one type of linker, and/or more than one type of active agent, n is an integer equal to or greater than 1. In some embodiments, n is an integer between 1 and 50, or between 2 and 20, or between 5 and 40. In some embodiments, n may be an integer of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 41, 43, 44, 45, 46, 47, 48, 49 or 50.

In some embodiments, an active agent, e.g., a guide RNA may be delivered to cells and tissues using viral, polymeric and liposomal formulations, cell-penetrating peptides, aptamers, ligands, or conjugates and antibody approaches. A moiety or ligand may direct guide RNAs to particular organ, tissue, or cell, for example, a liver hepatocyte, and may be referred to as a targeting moiety. In some embodiments, targeting moieties modify one or more properties of the attached molecule (e.g., a mRNA or a guide RNA), including but not limited to pharmacodynamic, pharmacokinetic, binding, absorption, cellular distribution, cellular uptake, charge and clearance.

Exemplary moieties that can be attached to a herein described active agent include, but are not limited to, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, dyes, lipid moieties such as a cholesterol moiety (Letsinger et al., Proc. Natl. Acad. Sci. USA, 1989, 86, 6553); cholic acid (Manoharan et al, Bioorg. Med. Chem. Lett., 1994, 4, 1053); a thioether, e.g., hexyl-S-tritylthiol (Manoharan et al, Ann. NY. Acad. Sci., 1992, 660, 306; Manoharan et al, Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al, EMBO J., 1991, 10, 111; Kabanov et al, FEBS Lett., 1990, 259, 327; Svinarchuk et al, Biochimie, 1993, 75, 49); a phospholipid, e.g., di-hexadecyl-rac-glycerol or triethylammonium-1,2-di-0-hexadecyl-rac-glycero-3-H-phosphonate (Manoharan et al, Tetrahedron Lett., 1995, 36, 3651; Shea et al, Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al, Biochim. Biophys. Acta, 1995, 1264, 229); or an octadecylamine or hexylamino-carbonyl-oxycholesterol moiety (Crooke et al, J. Pharmacol. Exp. Ther., 1996, 277, 923), all references incorporated herein in their entirety. Targeting moieties may include naturally occurring molecules, or recombinant or synthetic molecules, including, but not limited to, polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]2, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-0(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent), peptide and peptidomimetic ligands, including those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides; amphipathic peptides including, but not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H2A peptides, Xenopus peptides, esculentinis-1, and caerins. A peptidomimetic (also referred to herein as an oligopeptidomimetic) is a molecule capable of folding into a defined three-dimensional structure similar to a natural peptide. The peptide or peptidomimetic ligand or moiety can be about 5-50 amino acids long, e.g., about 5, 10, 15, 20, 25, 30, 35, 40, 45, or 50 amino acids long. In some embodiments, the targeting moiety may be other peptides such as somatostatin, octeotide, LHRH (luteinizing hormone releasing hormone), epidermal growth factor receptor (EGFR) binding peptide, aptide or bipodal peptide, RGD-containing peptides, a protein scaffold such as a fibronectin domain, a single domain antibody, a stable scFv, or other homing peptides. As non-limiting examples, a protein or peptide based targeting moiety may be a protein such as thrombospondin, tumor necrosis factors (TNF), annexin V, an interferon, angiostatin, endostatin, cytokine, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF). In some embodiments, the targeting moiety maybe an antibody, an antibody fragment, RGD peptide, folic acid or prostate specific membrane antigen (PSMA). In some embodiments, the protein scaffold may be an antibody-derived protein scaffold. Non-limiting examples include single domain antibody (dAbs), nanobody, single-chain variable fragment (scFv), antigen-binding fragment (Fab), Avibody, minibody, CH2D domain, Fcab, and bispecific T-cell engager (BiTE) molecules. In some embodiments, scFv is a stable scFv, wherein the scFv has hyperstable properties. In some embodiments, the nanobody may be derived from the single variable domain (VHH) of camelidae antibody.

In some embodiments, a targeting moiety recognizes or binds a target cell, a marker, or a molecule that is present exclusively or predominantly on the surface of particular cells. For example, a targeting moiety may bind a tumor antigen and direct the activating agent, e.g. a guide RNA-Cas complex to a malignant cell. In some embodiments, the targeting moiety recognizes an intra-cellular protein. In some embodiments, a targeting moiety directs a conjugate to specific tissues, cells, or locations in a cell (for e.g. trivalent N-acetylgalactosamine targets asialoglycoprotein receptor (ASGPR) on the surface of hepatocytes in mammals; Nair et el. J. Am. Chem. Soc. 2014, 136, 16958, see also U.S. Ser. No. 17/192,709). The targeting moiety can direct the conjugate in culture or in a whole organism, or both. In each case, the targeting moiety may bind to a receptor that is present on the surface of or within the targeted cell(s), wherein the targeting moiety binds to the receptor with an effective specificity, affinity and avidity. In other embodiments the targeting moiety targets the conjugate to a specific tissue such as the liver, kidney, lung or pancreas. In other cases, targeting moieties can direct the conjugate to cells of the reticular endothelial or lymphatic system, or to professional phagocytic cells such as macrophages or eosinophils. In some embodiments, the targeting moiety may recognize a RTK receptor, an EGF receptor, a serine or threonine kinase, G-protein coupled receptor, methyl CpG binding protein, cell surface glycoprotein, cancer stem cell antigen or marker, carbonic anhydrase, cytolytic T lymphocyte antigen, DNA methyltransferase, an ectoenzyme, a glycosylphosphatidylinositol-anchored co-receptor, a glypican-related integral membrane proteoglycan, a heat shock protein, a hypoxia induced protein, a multi drug resistant transporter, a Tumor-associated macrophage marker, a tumor associated carbohydrate antigen, a TNF receptor family member, a transmembrane protein, a tumor necrosis factor receptor superfamily member, a tumour differentiation antigen, a zinc dependent metallo-exopeptidase, a zinc transporter, a sodium-dependent transmembrane transport protein, a member of the SIGLEC family of lectins, or a matrix metalloproteinase.

Targeting moieties can be conjugated to nucleobases, sugar moieties, or internucleosidic linkages of a nucleic acid, e.g. a guide RNA or mRNA. Conjugation to purine nucleobases or derivatives thereof can occur at any position including, endocyclic and exocyclic atoms. In some embodiments, the 2-, 6-, 7-, or 8-positions of a purine nucleobase are attached to a moiety. Conjugation to pyrimidine nucleobases or derivatives thereof can also occur at any position. In some embodiments, the 2-, 5-, and 6-positions of a pyrimidine nucleobase can be substituted with a moiety. When a moiety is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The gamma-position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic. For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

A targeting moiety can be attached to an active agent or therapeutic nucleic acid described herein, such as a guide RNA, via RNA-RNA or RNA-DNA base pairing and hybridization. Not intended to be bound by any theories, a targeting moiety can comprise a coupling sequence that is capable of recognizing or binding an active agent, e.g., a guide RNA or a mRNA. In some embodiments, a targeting moiety comprises a coupling sequence capable of hybridizing to a 5′ portion, a 3′ portion, or a middle portion of a guide RNA. A guide RNA that hybridizes with a coupling sequence may comprise an extension. For example, the coupling sequence may be able to hybridize with the extension sequence of the guide RNA, thereby directing the guide RNA to desired in vivo, ex vivo, intercellular or intracellular locations, while the guide RNA functionality such as interaction with CRISPR enzyme or binding with target sequence(s) is not affected. In some embodiments, the guide RNA comprises an extension that includes a polynucleotide tail. In some embodiments, the guide nucleic acid comprises a poly(A) tail, a poly(U) tail, or a poly(T) tail capable of hybridizing with a poly(U) tail, a poly(A) tail, or a poly(A) tail of the coupling sequence respectively. In some embodiments, the guide nucleic acid may be a guide RNA that comprises the sequence of (A)n or (U)n. In some embodiments, the guide nucleic acid may comprise DNA and may comprise the sequence of (A)n or (T)n. In some embodiments, the coupling sequence may comprise the sequence of (A)n, (U)n or (T)n. As instantly used, n may be any integer between 1 and 200.

A coupling sequence may share sequence identity or complementarity with a nucleic acid active agent, or a portion thereof. In some embodiments, a coupling sequence may share at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% of identity with a guide RNA described herein, or a portion of such guide RNA. In some embodiments, a coupling sequence may share at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% of identity with the complementary sequence of a guide RNA described herein, or the complementary of a portion of such guide RNA. In some embodiments, the coupling sequence may comprise identity or complementarity with at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 contiguous nucleobases of the guide RNA or a complementary thereof.

In some embodiments, a targeting moiety may comprise or be associated with a coupling sequence that is chemically modified. In some embodiments, the coupling sequence comprises an extension that hybridizes with a therapeutic nucleic acid, e.g. a guide RNA, or a portion thereof. In some embodiments, the extension of the coupling sequence may be chemically modified. In some embodiments, the therapeutic nucleic acid, e.g. a guide RNA, may comprise an extension. In some embodiments, the extension of the guide RNA may be chemically modified.

A targeting moiety can be attached to a nucleic acid described herein via a carrier. The carriers may include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom. Representative U.S. patents that teach the preparation of conjugates of nucleic acids include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

A targeting moiety can be attached to an active agent, e. g. a guide RNA, via a linker. A linker may be bound to one or more active agents and a targeting moiety ligand to form a conjugate, wherein the conjugate releases at least one active agent, e.g. a guide RNA or guide RNA-Cas complex, upon delivery to a target cell. The linker may be attached to the targeting moiety and the active agent by functional groups independently selected from an ester bond, disulfide, amide, acylhydrazone, ether, carbamate, carbonate, and urea. Alternatively the linker can be attached to either the targeting moiety or the active agent by a non-cleavable group such as provided by the conjugation between a thiol and a maleimide, an azide and an alkyne. In some embodiments, a targeting moiety comprises one or more linkers. In some embodiments, one or more linkers as described herein connect a portion of the targeting moiety to a different portion of the targeting moiety.

The linkers can each independently comprises one or more functional groups selected from the group consisting of ethylene glycol, propylene glycol, amide, ester, ether, alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl. In some embodiments, a linker independently comprises phosphate, phosphorothioate, amide, ether, oxime, hydrazine or carbamate.

In some embodiments, the linker can independently comprise a C₁-C₁₀ straight chain alkyl, C₁-C₁₀ straight chain O-alkyl, C₁-C₁₀ straight chain substituted alkyl, C₁-C₁₀ straight chain substituted O-alkyl, C₄-C₁₃ branched chain alkyl, C₄-C₁₃ branched chain O-alkyl, C₂-C₁₂ straight chain alkenyl, C₂-C₁₂ straight chain O-alkenyl, aralkyl, C₃-C₁₂ straight chain substituted alkenyl, C₃-C₁₂ straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, urea, thiourea, amide, purine, pyrimidine, bypiridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C3 straight chain alkyl or a ketone. The alkyl chain of the linker can be substituted with one or more substituents or heteroatoms. In some embodiments, the alkyl chain of the linker may optionally be interrupted by one or more atoms or groups selected from —O—, —C(═O)—, —NR, —O—C(═O)—NR—, —S—, —S—S—.

In some embodiments, the linker may be cleavable and is cleaved to release the active agent. The cleavable functionality may be hydrolyzed in vivo or may be designed to be hydrolyzed enzymatically, for example by Cathepsin B. A “cleavable” linker, as used herein, refers to any linker which can be cleaved physically or chemically. Examples for physical cleavage may be cleavage by light, radioactive emission or heat, while examples for chemical cleavage include cleavage by re-dox-reactions, hydrolysis, pH-dependent cleavage.

Linkers may comprise a direct bond or an atom such as oxygen or sulfur, a unit such as NR¹, C(O), C(O)NH, SO, SO2, SO2NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO2, N(R′), C(O), substituted or unsubstituted aryl, substituted or unsubstituted heteroaryl, substituted or unsubstituted heterocyclic; where R′ is hydrogen, acyl, aliphatic or substituted aliphatic. In one embodiment, the linker is between 1-24 atoms, preferably 4-24 atoms, preferably 6-18 atoms, more preferably 8-18 atoms, and most preferably 8-16 atoms.

In one embodiment, the linker comprises at least one cleavable linking group. In certain embodiments, the linker is a branched linker. The branchpoint of the branched linker may be at least trivalent, but may be a tetravalent, pentavalent or hexavalent atom, or a group presenting such multiple valencies. In certain embodiments, the branchpoint is, —N, —N(O)—C, —O—C, —S—C, —SS—C, —C(O)N(O)—C, —OC(O)N(O)—C, —N(O)C(O)—C, or —N(O)C(O)O—C; wherein Q is independently for each occurrence H or optionally substituted alkyl. In other embodiment, the branchpoint is glycerol or glycerol derivative.

In one embodiment, a linker may be cleaved by an enzyme. As a non-limiting example, the linker may be a polypeptide moiety, e.g. AA in WO2010093395 to Govindan, the content of which is incorporated herein by reference in its entirety; that is cleavable by intracellular peptidase. Govindan teaches AA in the linker may be a di, tri, or tetrapeptide such as Ala-Leu, Leu-Ala-Leu, and Ala-Leu-Ala-Leu. In another example, the cleavable linker may be a branched peptide. The branched peptide linker may comprise two or more amino acid moieties that provide an enzyme cleavage site. Any branched peptide linker disclosed in WO 1998019705 to Dubowchik, the content of which is incorporated herein by reference in its entirety, may be used as a linker in the conjugate of the present disclosure. As another example, the linker may comprise a lysosomally cleavable polypeptide disclosed in U.S. Pat. No. 8,877,901 to Govindan et al., the content of which is incorporated herein by reference in its entirety. As another example, the linker may comprise a protein peptide sequence which is selectively enzymatically cleavable by tumor associated proteases, such as any Y and Z structures disclosed in U.S. Pat. No. 6,214,345 to Firestone et al, the content of which is incorporated herein by reference in its entirety.

In some embodiments, a linker may comprise a cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum). Cleavable linking groups may be susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular RNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

In some embodiments, a linker may comprise a phosphate based cleavable linking group. Phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups (i.e., phosphorus-containing linkages or phosphorus-containing linkers) are —P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. In some embodiments, phosphate-based linking groups are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—S—. In some embodiments, a phosphate-based linker is —O—P(O)(OH)—O—.

In some embodiments, a linker may comprise an acid cleavable linking group. Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

In some embodiments, a linker may comprise a ester based linking group. Ester-based cleavable linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

In some embodiments, a linker may comprise a peptide based linking group. Peptide-based cleavable linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

In some embodiments, the MA004 mRNA comprises a mRNA sequence of SEQ ID NO: 3 (Table 5). In some embodiments, the degree of similarity between a sequence of MA004 mRNA and another sequence may be at least 50% identical, 55% identical, 60% identical, 65% identical, 70% identical, 75% identical, 80% identical, 85% identical, 90% identical, 95% identical, 97% identical, 98% identical, 99% identical, or 100% identical. As used in Table 5, uppercase nucleotides, A, G, and C, indicate ribonucleotides adenine, guanine, and cytosine, respectively. As used in Table 5, lowercase u′ indicates N¹-methylpseudouridine.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The Smith Waterman algorithm can also be used to determine percent identity. Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Nat. Acad. Sci. USA., 89:10915-10919 (1992)) 3) gap penalty=12; and 4) gap length penalty=4. A program useful with these parameters can be publicly available as the “gap” program (Genetics Computer Group, Madison, Wis.). The aforementioned parameters are the default parameters for polypeptide comparisons (with no penalty for end gaps). Alternatively, polypeptide sequence identity can be calculated using the following equation: % identity−(the number of identical residues)/(alignment length in amino acid residues)*100. For this calculation, alignment length includes internal gaps but does not include terminal gaps.

TABLE 5
MA004 and MA079 mRNA sequences

| | | | SEQ ID |
|---|---|---|---|
| Region | Sequence | | NO. |
| Full | mRNA | AGGAAAu′AAGAGAGAAAAGAAGAGu′AAGAAGAAAu′A | 3 |
| sequence | | u′AAGAGCCACCAu′GAGCGAGGu′GGAGu′u′CAGCCA | |
| | | CGAGu′ACu′GGAu′GCGGCACGCCCu′GACCCu′GGCCA | |
| | | AGCGGGCCCGGGACGAGCGGGAGGu′GCCCGu′GGGCGCC | |
| | | Gu′GCu′GGu′GCu′GAACAACCGGGu′GAu′CGGCGAGG | |
| | | GCu′GGAACCGGGCCAu′CGGCCu′GCACGACCCCACCGC | |
| | | CCACGCCGAGAu′CAu′GGCCCu′GCGGCAGGGCGGCCu′ | |
| | | GGu′GAu′GCAGAACu′ACCGGCu′GAu′CGACGCCACCC | |
| | | u′Gu′ACGu′GACCu′u′CGAGCCCu′GCGu′GAu′Gu′G | |
| | | CGCCGGCGCCAu′GAu′CCACAGCCGGAu′CGGCCGGGu′ | |
| | | GGu′Gu′u′CGGCGu′GCGGAACGCCAAGACCGGCGCCGC | |
| | | CGGCAGCCu′GAu′GGACGu′GCu′GCACCACCCCGGCAu | |
| | | ′GAACCACCGGGu′GGAGAu′CACCGAGGGCAu′CCu′GG | |
| | | CCGACGAGu′GCGCCGCCCu′GCu′Gu′GCCGGu′u′Cu′ | |
| | | u′CCGGAu′GCCCCGGCGGGu′Gu′u′CAACGCCCAGAAG | |
| | | AAGGCCCAGAGCAGCACCGACAGCGGCGGCAGCAGCGGCG | |
| | | GCAGCAGCGGCAGCGAGACACCCGGCACCAGCGAGAGCGC | |
| | | CACCCCCGAGAGCAGCGGCGGCAGCAGCGGCGGCAGCGAC | |
| | | AAGAAGu′ACAGCAu′CGGCCu′GGCCAu′CGGCACCAAC | |
| | | AGCGu′GGGCu′GGGCCGu′GAu′CACCGACGAGu′ACAA | |
| | | GGu′GCCCAGCAAGAAGu′u′CAAGGu′GCu′GGGCAACA | |
| | | CCGACCGGCACAGCAu′CAAGAAGAACCu′GAu′CGGCGC | |
| | | CCu′GCu′Gu′u′CGACAGCGGCGAGACAGCCGAGGCCAC | |
| | | CCGGCu′GAAGCGGACCGCCCGGCGGCGGu′ACACCCGGC | |
| | | GGAAGAACCGGAu′Cu′GCu′ACCu′GCAGGAGAu′Cu′u | |
| | | ′CAGCAACGAGAu′GGCCAAGGu′GGACGACAGCu′u′Cu | |
| | | ′u′CCACCGGCu′GGAGGAGAGCu′u′CCu′GGu′GGAGG | |
| | | AGGACAAGAAGCACGAGCGGCACCCCAu′Cu′u′CGGCAA | |
| | | CAu′CGu′GGACGAGGu′GGCCu′ACCACGAGAAGu′ACC | |
| | | CCACCAu′Cu′ACCACCu′GCGGAAGAAGCu′GGu′GGAC | |
| | | AGCACCGACAAGGCCGACCu′GCGGCu′GAu′Cu′ACCu′ | |
| | | GGCCCu′GGCCCACAu′GAu′CAAGu′u′CCGGGGCCACu | |
| | | ′u′CCu′GAu′CGAGGGCGACCu′GAACCCCGACAACAGC | |
| | | GACGu′GGACAAGCu′Gu′u′CAu′CCAGCu′GGu′GCAG | |
| | | ACCu′ACAACCAGCu′Gu′u′CGAGGAGAACCCCAu′CAA | |
| | | CGCCAGCGGCGu′GGACGCCAAGGCCAu′CCu′GAGCGCC | |
| | | CGGCu′GAGCAAGAGCCGGCGGCu′GGAGAACCu′GAu′C | |
| | | GCCCAGCu′GCCCGGCGAGAAGAAGAACGGCCu′Gu′u′C | |
| | | GGCAACCu′GAu′CGCCCu′GAGCCu′GGGCCu′GACCCC | |
| | | CAACu′u′CAAGAGCAACu′u′CGACCu′GGCCGAGGACG | |
| | | CCAAGCu′GCAGCu′GAGCAAGGACACCu′ACGACGACGA | |
| | | CCu′GGACAACCu′GCu′GGCCCAGAu′CGGCGACCAGu′ | |
| | | ACGCCGACCu′Gu′u′CCu′GGCCGCCAAGAACCu′GAGC | |
| | | GACGCCAu′CCu′GCu′GAGCGACAu′CCu′GCGGGu′GA | |
| | | ACACCGAGAu′CACCAAGGCCCCCCu′GAGCGCCAGCAu′ | |
| | | GAu′CAAGCGGu′ACGACGAGCACCACCAGGACCu′GACC | |
| | | Cu′GCu′GAAGGCCCu′GGu′GCGGCAGCAGCu′GCCCGA | |
| | | GAAGu′ACAAGGAGAu′Cu′u′Cu′u′CGACCAGAGCAAG | |
| | | AACGGCu′ACGCCGGCu′ACAu′CGACGGCGGCGCCAGCC | |
| | | AGGAGGAGu′u′Cu′ACAAGu′u′CAu′CAAGCCCAu′CC | |
| | | u′GGAGAAGAu′GGACGGCACCGAGGAGCu′GCu′GGu′G | |
| | | AAGCu′GAACCGGGAGGACCu′GCu′GCGGAAGCAGCGGA | |
| | | CCu′u′CGACAACGGCAGCAu′CCCCCACCAGAu′CCACC | |
| | | u′GGGCGAGCu′GCACGCCAu′CCu′GCGGCGGCAGGAGG | |
| | | ACu′u′Cu′ACCCCu′u′CCu′GAAGGACAACCGGGAGAA | |
| | | GAu′CGAGAAGAu′CCu′GACCu′u′CCGGAu′CCCCu′A | |
| | | Cu′ACGu′GGGCCCCCu′GGCCCGGGGCAACAGCCGGu′u | |
| | | ′CGCCu′GGAu′GACCCGCAAGAGCGAGGAGACAAu′CAC | |
| | | CCCCu′GGAACu′u′CGAGGAGGu′GGu′GGACAAGGGCG | |
| | | CCAGCGCCCAGAGCu′u′CAu′CGAGCGGAu′GACCAACu | |
| | | ′u′CGACAAGAACCu′GCCCAACGAGAAGGu′GCu′GCCC | |
| | | AAGCACAGCCu′GCu′Gu′ACGAGu′ACu′u′CACCGu′G | |
| | | u′ACAACGAGCu′GACCAAGGu′GAAGu′ACGu′GACCGA | |
| | | GGGCAu′GCGGAAGCCCGCCu′u′CCu′GAGCGGCGAGCA | |
| | | GAAGAAGGCCAu′CGu′GGACCu′GCu′Gu′u′CAAGACC | |
| | | AACCGGAAGGu′GACCGu′GAAGCAGCu′GAAGGAGGACu | |
| | | ′ACu′u′CAAGAAGAu′CGAGu′GCu′u′CGACAGCGu′G | |
| | | GAGAu′CAGCGGCGu′GGAGGACCGGu′u′CAACGCCAGC | |
| | | Cu′GGGCACCu′ACCACGACCu′GCu′GAAGAu′CAu′CA | |
| | | AGGACAAGGACu′u′CCu′GGACAACGAGGAGAACGAGGA | |
| | | CAu′CCu′GGAGGACAu′CGu′GCu′GACCCu′GACCCu′ | |
| | | Gu′u′CGAGGACCGGGAGAu′GAu′CGAGGAGCGGCu′GA | |
| | | AGACCu′ACGCCCACCu′Gu′u′CGACGACAAGGu′GAu′ | |
| | | GAAGCAGCu′GAAGCGGCGGCGGu′ACACCGGCu′GGGGC | |
| | | CGGCu′GAGCCGGAAGCu′GAu′CAACGGCAu′CCGGGAC | |
| | | AAGCAGAGCGGCAAGACCAu′CCu′GGACu′u′CCu′CAA | |
| | | GAGCGACGGCu′u′CGCCAACCGGAACu′u′CAu′GCAGC | |
| | | u′GAu′CCACGACGACAGCCu′GACCu′u′CAAGGAGGAC | |
| | | Au′CCAGAAGGCCCAGGu′GAGCGGCCAGGGCGACAGCCu | |
| | | ′GCACGAGCACAu′CGCCAACCu′GGCCGGCAGCCCCGCC | |
| | | Au′CAAGAAGGGCAu′CCu′GCAGACCGu′GAAGGu′GGu | |
| | | ′GGACGAGCu′GGu′GAAGGu′GAu′GGGCCGGCACAAGC | |
| | | CCGAGAACAu′CGu′GAu′CGAGAu′GGCCCGGGAGAACC | |
| | | AGACCACCCAGAAGGGCCAGAAGAACAGCCGGGAGCGGAu | |
| | | ′GAAGCGGAu′CGAGGAGGGCAu′CAAGGAGCu′GGGCAG | |
| | | CCAGAu′CCu′GAAGGAGCACCCCGu′GGAGAACACCCAG | |
| | | Cu′GCAGAACGAGAAGCu′Gu′ACCu′Gu′ACu′ACCu′G | |
| | | CAGAACGGCCGGGACAu′Gu′ACGu′GGACCAGGAGCu′G | |
| | | GACAu′CAACCGGCu′GAGCGACu′ACGACGu′GGACCAC | |
| | | Au′CGu′GCCCCAGAGCu′u′CCu′GAAGGACGACAGCAu | |
| | | ′CGACAACAAGGu′GCu′GACCCGGAGCGACAAGAACCGG | |
| | | GGCAAGAGCGACAACGu′GCCCAGCGAGGAGGu′GGu′GA | |
| | | AGAAGAu′GAAGAACu′ACu′GGCGGCAGCu′GCu′GAAC | |
| | | GCCAAGCu′GAu′CACCCAGCGGAAGu′u′CGACAACCu′ | |
| | | GACCAAGGCCGAGCGGGGCGGCCu′GAGCGAGCu′GGACA | |
| | | AGGCCGGCu′u′CAu′CAAGCGGCAGCu′GGu′GGAGACA | |
| | | CGGCAGAu′CACCAAGCACGu′GGCCCAGAu′CCu′GGAC | |
| | | AGCCGGAu′GAACACCAAGu′ACGACGAGAACGACAAGCu | |
| | | ′GAu′CCGGGAGGu′GAAGGu′GAu′CACCCu′CAAGAGC | |
| | | AAGCu′GGu′GAGCGACu′u′CCGGAAGGACu′u′CCAGu | |
| | | ′u′Cu′ACAAGGu′GCGGGAGAu′CAACAACu′ACCACCA | |
| | | CGCCCACGACGCCu′ACCu′GAACGCCGu′GGu′GGGCAC | |
| | | CGCCCu′GAu′CAAGAAGu′ACCCCAAGCu′GGAGAGCGA | |
| | | Gu′u′CGu′Gu′ACGGCGACu′ACAAGGu′Gu′ACGACGu | |
| | | ′GCGGAAGAu′GAu′CGCCAAGAGCGAGCAGGAGAu′CGG | |
| | | CAAGGCCACCGCCAAGu′ACu′u′Cu′uCu′ACAGCAACA | |
| | | u′CAu′GAACu′u′Cu′u′CAAGACCGAGAu′CACCCu′G | |
| | | GCCAACGGCGAGAu′CCGGAAGCGGCCCCu′GAu′CGAGA | |
| | | CAAACGGCGAGACAGGCGAGAu′CGu′Gu′GGGACAAGGG | |
| | | CCGGGACu′u′CGCCACCGu′GCGGAAGGu′GCu′GAGCA | |
| | | u′GCCCCAGGu′GAACAu′CGu′GAAGAAGACCGAGGu′G | |
| | | CAGACCGGCGGCu′u′CAGCAAGGAGAGCAu′CCu′GCCC | |
| | | AAGCGGAACAGCGACAAGCu′GAu′CGCCCGGAAGAAGGA | |
| | | Cu′GGGACCCCAAGAAGu′ACGGCGGCu′u′CGACAGCCC | |
| | | CACCGu′GGCCu′ACAGCGu′GCu′GGu′GGu′GGCCAAG | |
| | | Gu′GGAGAAGGGCAAGAGCAAGAAGCu′CAAGAGCGu′GA | |
| | | AGGAGCu′GCu′GGGCAu′CACCAu′CAu′GGAGCGGAGC | |
| | | AGCu′u′CGAGAAGAACCCCAu′CGACu′u′CCu′GGAGG | |
| | | CCAAGGGCu′ACAAGGAGGu′GAAGAAGGACCu′GAu′CA | |
| | | u′CAAGCu′GCCCAAGu′ACAGCCu′Gu′u′CGAGCu′GG | |
| | | AGAACGGCCGGAAGCGGAu′GCu′GGCCAGCGCCGGCGAG | |
| | | Cu′GCAGAAGGGCAACGAGCu′GGCCCu′GCCCAGCAAGu | |
| | | ′ACGu′GAACu′u′CCu′Gu′ACCu′GGCCAGCCACu′AC | |
| | | GAGAAGCu′GAAGGGCAGCCCCGAGGACAACGAGCAGAAG | |
| | | CAGCu′Gu′u′CGu′GGAGCAGCACAAGCACu′ACCu′GG | |
| | | ACGAGAu′CAu′CGAGCAGAu′CAGCGAGu′u′CAGCAAG | |
| | | CGGGu′GAu′CCu′GGCCGACGCCAACCu′GGACAAGGu′ | |
| | | GCu′GAGCGCCu′ACAACAAGCACCGGGACAAGCCCAu′C | |
| | | CGGGAGCAGGCCGAGAACAu′CAu′CCACCu′Gu′u′CAC | |
| | | CCu′GACCAACCu′GGGCGCCCCCGCCGCCu′u′CAAGu′ | |
| | | ACu′u′CGACACCACCAu′CGACCGGAAGCGGu′ACACCA | |
| | | GCACCAAGGAGGu′GCu′GGACGCCACCCu′GAu′CCACC | |
| | | AGAGCAu′CACCGGCCu′Gu′ACGAGACACGGAu′CGACC | |
| | | u′GAGCCAGCu′GGGCGGCGACGAGGGCGCCGACAAGCGG | |
| | | ACCGCCGACGGCAGCGAGu′u′CGAGAGCCCCAAGAAGAA | |
| | | GCGGAAGGu′Gu′GAGCGGCCGCu′u′AAu′u′AAGCu′G | |
| | | CCu′u′Cu′GCGGGGCu′u′GCCu′u′Cu′GGCCAu′GCC | |
| | | Cu′u′Cu′u′Cu′Cu′CCCu′u′GCACCu′Gu′ACCu′Cu | |
| | | ′u′GGu′Cu′u′u′GAAu′AAAGCCu′GAGu′AGGAAGu′ | |
| | | Cu′AGA | |
| Full | mRNA | AGGACu′CCu′CCCCAu′CCu′Cu′CCCu′Cu′Gu′CCCu | SEQ ID |
| Sequence | | ′Cu′Gu′CCCu′Cu′GACCCu′GCACu′Gu′CCCAGCACC | NO: 4 |
| | | Au′GAGCGAGGu′GGAGu′u′CAGCCACGAGu′ACu′GGA | |
| | | u′GCGCCACGCCCu′GACCCu′GGCCAAGCGCGCCCGCGA | |
| | | CGAGCGCGAGGu′GCCCGu′GGGCGCCGu′GCu′GGu′GC | |
| | | u′GAACAACCGCGu′GAu′CGGCGAGGGCu′GGAACCGCG | |
| | | CCAu′CGGCCu′GCACGACCCCACCGCCCACGCCGAGAu′ | |
| | | CAu′GGCCCu′GCGCCAGGGCGGCCu′GGu′GAu′GCAGA | |
| | | ACu′ACCGCCu′GAu′CGACGCCACCCu′Gu′ACGu′GAC | |
| | | Cu′u′CGAGCCCu′GCGu′GAu′Gu′GCGCCGGCGCCAu′ | |
| | | GAu′CCACAGCCGCAu′CGGCCGCGu′GGu′Gu′u′CGGC | |
| | | Gu′GCGCAACGCCAAGACCGGCGCCGCCGGCAGCCu′GAu | |
| | | ′GGACGu′GCu′GCACCACCCCGGCAu′GAACCACCGCGu | |
| | | ′GGAGAu′CACCGAGGGCAu′CCu′GGCCGACGAGu′GCG | |
| | | CCGCCCu′GCu′Gu′GCCGCu′u′Cu′u′CCGCAu′GCCC | |
| | | CGCCGCGu′Gu′u′CAACGCCCAGAAGAAGGCCCAGAGCA | |
| | | GCACCGACAGCGGCGGCAGCAGCGGCGGCAGCAGCGGCAG | |
| | | CGAGACACCCGGCACCAGCGAGAGCGCCACCCCCGAGAGC | |
| | | AGCGGCGGCAGCAGCGGCGGCAGCGACAAGAAGu′ACAGC | |
| | | Au′CGGCCu′GGCCAu′CGGCACCAACAGCGu′GGGCu′G | |
| | | GGCCGu′GAu′CACCGACGAGu′ACAAGGu′GCCCAGCAA | |
| | | GAAGu′u′CAAGGu′GCu′GGGCAACACCGACCGCCACAG | |
| | | CAu′CAAGAAGAACCu′GAu′CGGCGCCCu′GCu′Gu′u′ | |
| | | CGACAGCGGCGAGACAGCCGAGGCCACCCGCCu′GAAGCG | |
| | | CACCGCCCGCCGCCGCu′ACACCCGCCGCAAGAACCGCAu | |
| | | ′Cu′GCu′ACCu′GCAGGAGAu′Cu′u′CAGCAACGAGAu | |
| | | ′GGCCAAGGu′GGACGACAGCu′u′Cu′u′CCACCGCCu′ | |
| | | GGAGGAGAGCu′u′CCu′GGu′GGAGGAGGACAAGAAGCA | |
| | | CGAGCGCCACCCCAu′Cu′u′CGGCAACAu′CGu′GGACG | |
| | | AGGu′GGCCu′ACCACGAGAAGu′ACCCCACCAu′Cu′AC | |
| | | CACCu′GCGCAAGAAGCu′GGu′GGACAGCACCGACAAGG | |
| | | CCGACCu′GCGCCu′GAu′Cu′ACCu′GGCCCu′GGCCCA | |
| | | CAu′GAu′CAAGu′u′CCGCGGCCACu′u′CCu′GAu′CG | |
| | | AGGGCGACCu′GAACCCCGACAACAGCGACGu′GGACAAG | |
| | | Cu′Gu′u′CAu′CCAGCu′GGu′GCAGACCu′ACAACCAG | |
| | | Cu′Gu′u′CGAGGAGAACCCCAu′CAACGCCAGCGGCGu′ | |
| | | GGACGCCAAGGCCAu′CCu′GAGCGCCCGCCu′GAGCAAG | |
| | | AGCCGCCGCCu′GGAGAACCu′GAu′CGCCCAGCu′GCCC | |
| | | GGCGAGAAGAAGAACGGCCu′Gu′u′CGGCAACCu′GAu′ | |
| | | CGCCCu′GAGCCu′GGGCCu′GACCCCCAACu′u′CAAGA | |
| | | GCAACu′u′CGACCu′GGCCGAGGACGCCAAGCu′GCAGC | |
| | | u′GAGCAAGGACACCu′ACGACGACGACCu′GGACAACCu | |
| | | ′GCu′GGCCCAGAu′CGGCGACCAGu′ACGCCGACCu′Gu | |
| | | ′u′CCu′GGCCGCCAAGAACCu′GAGCGACGCCAu′CCu′ | |
| | | GCu′GAGCGACAu′CCu′GCGCGu′GAACACCGAGAu′CA | |
| | | CCAAGGCCCCCCu′GAGCGCCAGCAu′GAu′CAAGCGCu′ | |
| | | ACGACGAGCACCACCAGGACCu′GACCCu′GCu′GAAGGC | |
| | | CCu′GGu′GCGCCAGCAGCu′GCCCGAGAAGu′ACAAGGA | |
| | | GAu′Cu′u′Cu′u′CGACCAGAGCAAGAACGGCu′ACGCC | |
| | | GGCu′ACAu′CGACGGCGGCGCCAGCCAGGAGGAGu′u′C | |
| | | u′ACAAGu′u′CAu′CAAGCCCAu′CCu′GGAGAAGAu′G | |
| | | GACGGCACCGAGGAGCu′GCu′GGu′GAAGCu′GAACCGC | |
| | | GAGGACCu′GCu′GCGCAAGCAGCGCACCu′u′CGACAAC | |
| | | GGCAGCAu′CCCCCACCAGAu′CCACCu′GGGCGAGCu′G | |
| | | CACGCCAu′CCu′GCGCCGCCAGGAGGACu′u′Cu′ACCC | |
| | | Cu′u′CCu′GAAGGACAACCGCGAGAAGAu′CGAGAAGAu | |
| | | ′CCu′GACCu′u′CCGCAu′CCCCu′ACu′ACGu′GGGCC | |
| | | CCCu′GGCCCGCGGCAACAGCCGCu′u′CGCCu′GGAu′G | |
| | | ACCCGCAAGAGCGAGGAGACAAu′CACCCCCu′GGAACu′ | |
| | | u′CGAGGAGGu′GGu′GGACAAGGGCGCCAGCGCCCAGAG | |
| | | Cu′u′CAu′CGAGCGCAu′GACCAACu′u′CGACAAGAAC | |
| | | Cu′GCCCAACGAGAAGGu′GCu′GCCCAAGCACAGCCu′G | |
| | | Cu′Gu′ACGAGu′ACu′u′CACCGu′Gu′ACAACGAGCu′ | |
| | | GACCAAGGu′GAAGu′ACGu′GACCGAGGGCAu′GCGCAA | |
| | | GCCCGCCu′u′CCu′GAGCGGCGAGCAGAAGAAGGCCAu′ | |
| | | CGu′GGACCu′GCu′Gu′u′CAAGACCAACCGCAAGGu′G | |
| | | ACCGu′GAAGCAGCu′GAAGGAGGACu′ACu′u′CAAGAA | |
| | | GAu′CGAGu′GCu′u′CGACAGCGu′GGAGAu′CAGCGGC | |
| | | Gu′GGAGGACCGCu′u′CAACGCCAGCCu′GGGCACCu′A | |
| | | CCACGACCu′GCu′GAAGAu′CAu′CAAGGACAAGGACu′ | |
| | | u′CCu′GGACAACGAGGAGAACGAGGACAu′CCu′GGAGG | |
| | | ACAu′CGu′GCu′GACCCu′GACCCu′Gu′u′CGAGGACC | |
| | | GCGAGAu′GAu′CGAGGAGCGCCu′GAAGACCu′ACGCCC | |
| | | ACCu′Gu′u′CGACGACAAGGu′GAu′GAAGCAGCu′GAA | |
| | | GCGCCGCCGCu′ACACCGGCu′GGGGCCGCCu′GAGCCGC | |
| | | AAGCu′GAu′CAACGGCAu′CCGCGACAAGCAGAGCGGCA | |
| | | AGACCAu′CCu′GGACu′u′CCu′CAAGAGCGACGGCu′u | |
| | | ′CGCCAACCGCAACu′u′CAu′GCAGCu′GAu′CCACGAC | |
| | | GACAGCCu′GACCu′u′CAAGGAGGACAu′CCAGAAGGCC | |
| | | CAGGu′GAGCGGCCAGGGCGACAGCCu′GCACGAGCACAu | |
| | | ′CGCCAACCu′GGCCGGCAGCCCCGCCAu′CAAGAAGGGC | |
| | | Au′CCu′GCAGACCGu′GAAGGu′GGu′GGACGAGCu′GG | |
| | | u′GAAGGu′GAu′GGGCCGCCACAAGCCCGAGAACAu′CG | |
| | | u′GAu′CGAGAu′GGCCCGCGAGAACCAGACCACCCAGAA | |
| | | GGGCCAGAAGAACAGCCGCGAGCGCAu′GAAGCGCAu′CG | |
| | | AGGAGGGCAu′CAAGGAGCu′GGGCAGCCAGAu′CCu′GA | |
| | | AGGAGCACCCCGu′GGAGAACACCCAGCu′GCAGAACGAG | |
| | | AAGCu′Gu′ACCu′Gu′ACu′ACCu′GCAGAACGGCCGCG | |
| | | ACAu′Gu′ACGu′GGACCAGGAGCu′GGACAu′CAACCGC | |
| | | Cu′GAGCGACu′ACGACGu′GGACCACAu′CGu′GCCCCA | |
| | | GAGCu′u′CCu′GAAGGACGACAGCAu′CGACAACAAGGu | |
| | | ′GCu′GACCCGCAGCGACAAGAACCGCGGCAAGAGCGACA | |
| | | ACGu′GCCCAGCGAGGAGGu′GGu′GAAGAAGAu′GAAGA | |
| | | ACu′ACu′GGCGCCAGCu′GCu′GAACGCCAAGCu′GAu′ | |
| | | CACCCAGCGCAAGu′u′CGACAACCu′GACCAAGGCCGAG | |
| | | CGCGGCGGCCu′GAGCGAGCu′GGACAAGGCCGGCu′u′C | |
| | | Au′CAAGCGCCAGCu′GGu′GGAGACACGCCAGAu′CACC | |
| | | AAGCACGu′GGCCCAGAu′CCu′GGACAGCCGCAu′GAAC | |
| | | ACCAAGu′ACGACGAGAACGACAAGCu′GAu′CCGCGAGG | |
| | | u′GAAGGu′GAu′CACCCu′CAAGAGCAAGCu′GGu′GAG | |
| | | CGACu′u′CCGCAAGGACu′u′CCAGu′u′Cu′ACAAGGu | |
| | | ′GCGCGAGAu′CAACAACu′ACCACCACGCCCACGACGCC | |
| | | u′ACCu′GAACGCCGu′GGu′GGGCACCGCCCu′GAu′CA | |
| | | AGAAGu′ACCCCAAGCu′GGAGAGCGAGu′u′CGu′Gu′A | |
| | | CGGCGACu′ACAAGGu′Gu′ACGACGu′GCGCAAGAu′GA | |
| | | u′CGCCAAGAGCGAGCAGGAGAu′CGGCAAGGCCACCGCC | |
| | | AAGu′ACu′u′Cu′u′Cu′ACAGCAACAu′CAu′GAACu′ | |
| | | u′Cu′u′CAAGACCGAGAu′CACCCu′GGCCAACGGCGAG | |
| | | Au′CCGCAAGCGCCCCCu′GAu′CGAGACAAACGGCGAGA | |
| | | CAGGCGAGAu′CGu′Gu′GGGACAAGGGCCGCGACu′u′C | |
| | | GCCACCGu′GCGCAAGGu′GCu′GAGCAu′GCCCCAGGu′ | |
| | | GAACAu′CGu′GAAGAAGACCGAGGu′GCAGACCGGCGGC | |
| | | u′u′CAGCAAGGAGAGCAu′CCu′GCCCAAGCGCAACAGC | |
| | | GACAAGCu′GAu′CGCCCGCAAGAAGGACu′GGGACCCCA | |
| | | AGAAGu′ACGGCGGCu′u′CGACAGCCCCACCGu′GGCCu | |
| | | ′ACAGCGu′GCu′GGu′GGu′GGCCAAGGu′GGAGAAGGG | |
| | | CAAGAGCAAGAAGCu′CAAGAGCGu′GAAGGAGCu′GCu′ | |
| | | GGGCAu′CACCAu′CAu′GGAGCGCAGCAGCu′u′CGAGA | |
| | | AGAACCCCAu′CGACu′u′CCu′GGAGGCCAAGGGCu′AC | |
| | | AAGGAGGu′GAAGAAGGACCu′GAu′CAu′CAAGCu′GCC | |
| | | CAAGu′ACAGCCu′Gu′u′CGAGCu′GGAGAACGGCCGCA | |
| | | AGCGCAu′GCu′GGCCAGCGCCGGCGAGCu′GCAGAAGGG | |
| | | CAACGAGCu′GGCCCu′GCCCAGCAAGu′ACGu′GAACu′ | |
| | | u′CCu′Gu′ACCu′GGCCAGCCACu′ACGAGAAGCu′GAA | |
| | | GGGCAGCCCCGAGGACAACGAGCAGAAGCAGCu′Gu′u′C | |
| | | Gu′GGAGCAGCACAAGCACu′ACCu′GGACGAGAu′CAu′ | |
| | | CGAGCAGAu′CAGCGAGu′u′CAGCAAGCGCGu′GAu′CC | |
| | | u′GGCCGACGCCAACCu′GGACAAGGu′GCu′GAGCGCCu | |
| | | ′ACAACAAGCACCGCGACAAGCCCAu′CCGCGAGCAGGCC | |
| | | GAGAACAu′CAu′CCACCu′Gu′u′CACCCu′GACCAACC | |
| | | u′GGGCGCCCCCGCCGCCu′u′CAAGu′ACu′u′CGACAC | |
| | | CACCAu′CGACCGCAAGCGCu′ACACCAGCACCAAGGAGG | |
| | | u′GCu′GGACGCCACCCu′GAu′CCACCAGAGCAu′CACC | |
| | | GGCCu′Gu′ACGAGACACGCAu′CGACCu′GAGCCAGCu′ | |
| | | GGGCGGCGACGAGGGCGCCGACAAGCGCACCGCCGACGGC | |
| | | AGCGAGu′u′CGAGAGCCCCAAGAAGAAGCGCAAGGu′Gu | |
| | | ′AAAGu′Gu′CCAGACCAu′u′Gu′Cu′u′CCAACCCCAG | |
| | | Cu′GGCCu′Cu′AGAACACCCACu′GGCCAGu′CCu′AGA | |
| | | GCu′CCu′Gu′CCCu′ACCCACu′Cu′u′u′GCu′ACAAu | |
| | | ′AAAu′GCu′GAAu′GAAu′CCAu′Cu′AGA | |

### Target Sequences

The present disclosure provides active agents or therapeutic agents, such as genome editing compositions, and methods and compositions for targeted delivery thereof. The therapeutic agents described herein may comprise genome editing composition directed to and modify, alter, or cleave a target sequence on a target nucleic acid molecule. For example, the active agent may comprise a nucleic acid or a nucleic acid-protein complex capable of effecting a modification to a target sequence.

The target sequence may be a DNA sequence or a RNA sequence. In some embodiments, the active agent or therapeutic agent may comprise a RNA interference factor. In some embodiments, the active agent may comprise a siRNA, shRNA, antisense oligonucleotide, microRNA, anti-microRNA or antimir, supermir, antagomir, ribozyme, triplex-forming oligonucleotide, decoy oligonucleotide, splice-switching oligonucleotide, immunostimulatory oligonucleotide, RNA activator, or a Ul adaptor. The active agent may recognize the target sequence and mediate cleavage and/or degradation of the target sequence. In some embodiments, the active or therapeutic agent may comprise a guide RNA. The guide RNA may be complexed with a nucleic acid guided programmable nuclease, such as a CRISPR enzyme, such as a Cas9, or a fusion protein thereof further comprising a functional domain. The target sequence may be recognized by the nucleic acid guided programmable nuclease domain. The target sequence may be cleaved by the nucleic acid guided programmable nuclease domain and/or modified by the functional domain, such as a deaminase domain, a methylase domain, a methyltransferase domain, an activation domain, a repressor domain, a nuclease domain, a transposase domain, or a recombinase domain. In some embodiments, a Cas9 protein may be directed by a guide RNA to a target sequence of a target nucleic acid molecule, where the guide RNA hybridizes with and the Cas protein cleaves the target sequence. In some embodiments, the target sequence may be complementary to the targeting sequence of the guide RNA. In some embodiments, the degree of complementarity between a targeting sequence of a guide RNA and its corresponding target sequence may be about 50%, 55%, 60%, 65%, 70%, 75%, 80%, 85%, 90%, 95%, 97%, 98%, 99%, or 100%. In some embodiments, the target sequence and the targeting sequence of the guide RNA may be 100% complementary. In other embodiments, the target sequence and the targeting sequence of the guide RNA may contain at least one mismatch. For example, the target sequence and the targeting sequence of the guide RNA may contain 1, 2, 3, 4, 5, 6, 7, 8, 9, or 10 mismatches. In some embodiments, the target sequence and the targeting sequence of the guide RNA may contain 1-6 mismatches. In some embodiments, the target sequence and the targeting sequence of the guide RNA may contain 5 or 6 mismatches.

The length of the target sequence may depend on the nuclease system used. For example, the target sequence for a CRISPR/Cas system may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the target sequence may comprise 18-24 nucleotides in length. In some embodiments, the target sequence may comprise 19-21 nucleotides in length. In some embodiments, the target sequence may comprise 20 nucleotides in length. When nickases are used, the target sequence may comprise a pair of target sequences recognized by a pair of nickases on opposite strands of the DNA molecule.

In some embodiments, the active or therapeutic agent may comprise a meganuclease system. the target sequence for a meganuclease may comprise 12-40 or more nucleotides in length. When ZFNs are used, the target sequence may comprise two half target sequences recognized by a pair of ZFNs on opposite strands of the DNA molecule, with an interconnecting sequence in between. In some embodiments, each half target sequence for ZFNs may independently comprise 9, 12, 15, 18, or more nucleotides in length. In some embodiments, the interconnecting sequence for ZFNs may comprise 4-20 nucleotides in length. In some embodiments, the interconnecting sequence for ZFNs may comprise 5-7 nucleotides in length.

When TALENs are used, the target sequence may similarly comprise two half target sequences recognized by a pair of TALENs on opposite strands of the DNA molecule, with an interconnecting sequence in between. In some embodiments, each half target sequence for TALENs may independently comprise 10-20 or more nucleotides in length. In some embodiments, the interconnecting sequence for TALENs may comprise 4-20 nucleotides in length. In some embodiments, the interconnecting sequence for TALENs may comprise 12-19 nucleotides in length.

In some embodiments, the target sequence may be adjacent to a protospacer adjacent motif (PAM), a short sequence recognized by a CRISPR/Cas complex. The protospacer adjacent motif, or PAM, is essential for target binding for CRISPR/Cas complexes. Typically, a PAM is a 2-6 base pair DNA sequence immediately following the DNA target sequence of the Cas nuclease. The PAM may be a 5′ PAM or a 3′ PAM. The exact sequence of PAM depends on the type of Cas protein. For example, a typical SpCas9 binding requires a 3′-NGG-5′ PAM, also known as a canonical PAM, where the N is any one of A, G, C, or T. A SpCas9 with certain amino acid substitutions, e.g. D1135E, R1335Q, G1218R, and/or T1337R can recognize a NGA PAM or a NGCG PAM. A SaCas9 binding requires a 3′-NNGRRT-5′ PAM. A SaCas9 with certain amino substitutions, e.g., K781E, K697N, H1014R, can recognize a NNNRRT PAM.

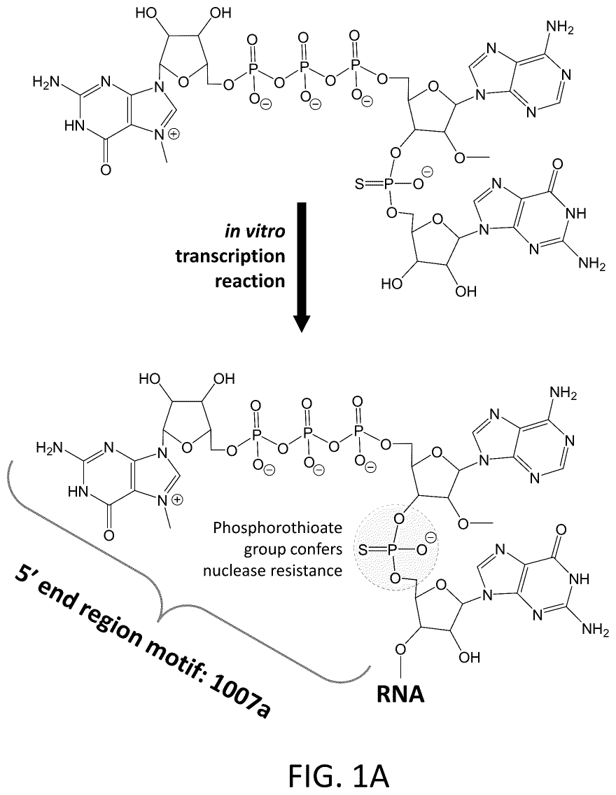

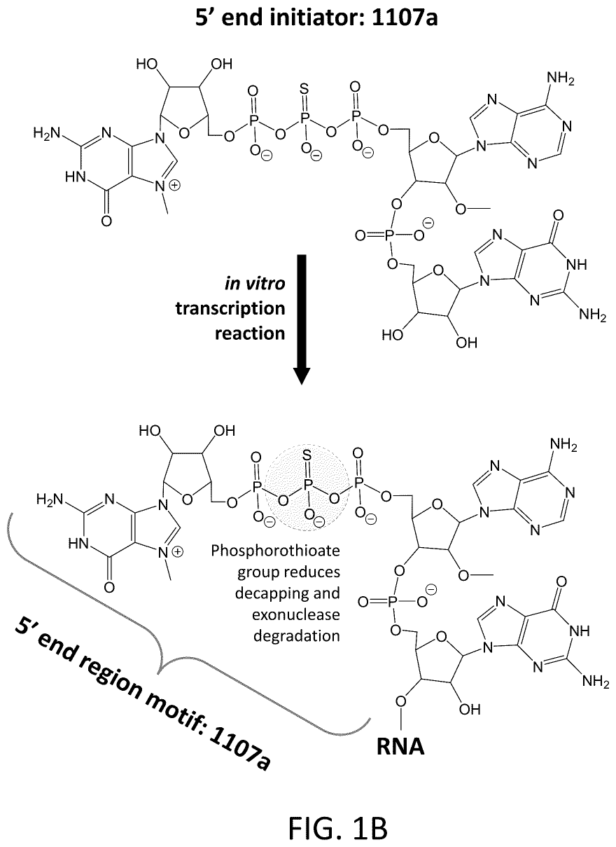

In some embodiments, the PAM may be adjacent to or within 1, 2, 3, or 4, nucleotides of the 3′ end of the target sequence. The length and the sequence of the PAM may depend on the Cas9 protein used. For example, the PAM may be selected from a consensus or a particular PAM sequence for a specific Cas9 protein or Cas9 ortholog, including those disclosed in FIG. 1 of Ran et al., Nature, 520: 186-191 (2015), which is incorporated herein by reference. In some embodiments, the PAM may comprise 2, 3, 4, 5, 6, 7, 8, 9, or 10 nucleotides in length. Non-limiting exemplary PAM sequences include NGG, NGGNG, NG, NAAAAN, NNAAAAW, NNNNACA, GNNNCNNA, and NNNNGATT (wherein N is defined as any nucleotide, and W is defined as either A or T). In some embodiments, the PAM sequence may be NGG. In some embodiments, the PAM sequence may be NGGNG. In some embodiments, the PAM sequence may be NNAAAAW. Additional evolved Cas variants and PAM sequences as described in Hu et al., Evolved Cas9 variants with broad PAM compatibility and high DNA specificity, Nature 2018 556(7699): 57-63 is incorporated herein in its entirety.

The target nucleic acid molecule may be any DNA or RNA molecule that is endogenous or exogenous to a cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the target nucleic acid molecule may be a plasmid, a genomic DNA, or a chromosome from a cell or in the cell. In some embodiments, the target sequence of the target nucleic acid molecule may be a genomic sequence from a cell or in the cell. In some embodiments, the cell may be a prokaryotic cell. In other embodiments, the cell may be a eukaryotic cell. In some embodiments, the eukaryotic cell may be a mammalian cell. In some embodiments, the eukaryotic cell may be a rodent cell. In some embodiments, the eukaryotic cell may be a human cell. In some embodiments, the eukaryotic cell may be a liver cell. In some embodiments, the eukaryotic cell may be a hepatocyte. In some embodiments, the eukaryotic cell may be a parenchymal cell, a sinusoidal endothelial cell, a phagocytic Kupffer cell, or a stellate cell. In further embodiments, the target sequence may be a viral sequence. In yet other embodiments, the target sequence may be a synthesized sequence. In some embodiments, the target sequence may be on a eukaryotic chromosome, such as a human chromosome.

In some embodiments, the target sequence may be located in a coding sequence of a gene, an intron sequence of a gene, a transcriptional control sequence of a gene, a translational control sequence of a gene, or a non-coding sequence between genes. In some embodiments, the gene may be a protein coding gene. In other embodiments, the gene may be a non-coding RNA gene. In some embodiments, the target sequence may comprise all or a portion of a disease-associated gene. In some embodiments, the target sequence may comprise all or a portion of a gene associated with a coronary disease. In some embodiments, the target sequence may comprise at least a portion of a gene encoding an apolipoprotein. In some embodiments, the target sequence may comprise at least a portion of a gene encoding Proprotein Convertase Subtilisin/Kexin Type 9 (PCSK9), Angiopoietin-like protein 3 (ANGPTL3), Apoliporotein C3 (APOC3), Apolipoprotein A (LPA), LDL Receptor (LDL-R), Myosin regulatory light chain interacting protein or inducible degrader of the LDL receptor (IDOL), Transthyretin (TTR), Antithrombin, AT3, Apolipoprotein B (ApoB), or Hup. The present disclosure also contemplates the use of modification to manipulate expression and activity of target genes, e.g., use of destabilizing mutations to counteract the effect of a gain-of-function PCSK9 variant.

The nucleic acid sequences of exemplary target genes and of other members of the family of human origin and those of a number of animals are publicly available, e.g., from the NCBI website or ENSEMBL website. Examples include, but are not limited to the following sequences: Wild Type PCSK9 Gene (NG 009061.1), Homo sapiens proprotein convertase subtilisin/kexin type 9 (PCSK9), RefSeqGene (LRG 275) on chromosome 1; human APOC3 Deposit No. NP_000031.1, GenBank Accession Nos.: NG_008949.1; Mouse, rat and monkey APOC3 nucleic acid sequences Ensembl accession number ENSMUSG00000032081, ENSRNOG00000047503, and ENSMFAG00000001837, respectively; human ANGPTL3 is provided, for example, in NG_028169.1; Mouse, rat, and monkey ANGPTL3 nucleic acid sequences Ensembl accession number ENSMUSG00000028553, ENSRNOG000000008638, and ENSMFAG00000007083, respectively; each of which sequences are incorporated herein its entirety.

In some embodiments, contacting a target sequences with the genome editing composition described herein leads to a base editing event within or adjacent to the target sequence. For example, a target base (e.g. a C base) within or adjacent to a target sequence may be converted to a T base as the result of contact with the genome editing composition as disclosed in the present disclosure comprising a fusion protein comprising a nucleic acid guided nuclease domain and a deaminase domain. In some embodiments, the target base is located upstream (5′ end of) of the PAM. In some embodiments, the target base is located at a position 1, 2, 3, 4, 5, 6, 7, 8, 9, 10, 11, 12, 13, 14, 15, 16, 17, 18, 19, 20, 21, 22, 23, 24, 25, 26, 28, 29, 30, 31, 32, 33, 34, 35, 36, 37, 38, 39, 40, 41, 42, 43, 44, 45, 46, 47, 48, 49, or 50 base pairs upstream (5′ end of) the PAM. In some embodiments, the target base is located at a position within 13 to 17 base pairs upstream (5′ end) of the PAM. In some embodiments, the target base is located at a position outside of 13 to 17 base pairs upstream (5′ end) of the PAM. In some embodiments, the target base pair is located at a position 10-15 base pairs upstream (5′ end) of the PAM. In some embodiments, the target base is located at a position 11-12 base pairs upstream of the PAM. In some embodiments, the target base is 11 base pairs upstream (5′ end) of the PAM. In some embodiments, the target base is located in the coding region (e.g., an exon) of the target sequence, e.g. the ANGPTL3 encoding polynucleotide (e.g., the ANGPTL3 gene locus). For example, conversion of a base in the coding region of the ANGPTL3 gene locus may result in an amino acid change in the ANGPTL3 protein sequence, i.e., a mutation. In some embodiments, the mutation is a loss of function mutation. In some embodiments, a mutation may introduce a pre-mature stop codon into the coding region the target sequence, e.g. coding region of the ANGPTL3 gene. In some embodiments, a loss-of-function mutation is a naturally occurring loss-of-function mutation. In some embodiments, the mutation is located in the coding region of the PCSK9 gene, e.g. a G106R, L253F, A443T, R93C, G24D, S47F, R46H, S 153N, or H193Y mutation. In some embodiments, the loss-of-function mutation introduces a pre-mature stop codon into the coding region of the ANGPTL3 gene. In some embodiments, a loss of function mutation may be introduced into the coding region of a APOC3 gene, e.g. a R19X mutation. In some embodiments, a loss of function mutation may be introduced into a Low-Density Lipoprotein Receptor (LDL-R) protein. In some embodiments, a loss of function mutation may be introduced into a Inducible Degrader of the LDL Receptor (IDOL) protein.

In some embodiments, a target sequence is located in a non-coding region of the target sequence, e.g., in an intron or a splicing site of a target gene. In some embodiments, a target sequence is located in a splicing site and the editing of such target base causes alternative splicing of the target gene mRNA. In some embodiments, the alternative splicing leads to leading to loss-of-function mutants. In some embodiments, the alternative splicing leads to introduction of a premature stop codon or a frameshift in the target mRNA, resulting in truncated, unstable, or folding-defective polypeptides. In some embodiments, stop codons may be introduced into the coding sequence of a apolipoprotein encoding gene upstream of the normal stop codon (referred to as a “premature stop codon”). In some embodiments, stop codons may be introduced into the coding region of the target gene. Premature stop codons cause premature translation termination, in turn resulting in truncated and nonfunctional proteins and induces rapid degradation of the mRNA via the non-sense mediated mRNA decay pathway. See, e.g., Baker et al., Current Opinion in Cell Biology 16 (3): 293-299, 2004; Chang et al, Annual Review of Biochemistry 76: 51-74, 2007; and Behm-Ansmant et ah, Genes & Development 20 (4): 391-398, 2006, each of which is incorporated herein by reference. The genome editing composition described herein may be used to introduce multiple editing events to the target sequence. For example, the genome editing composition may comprise a nucleic acid guide programmable nuclease that induces double strand breaks, deletions, insertions, frameshift, reversions, or other alterations in the target gene. For example, the genome editing composition may comprise a nucleic acid guided programmable nuclease-deaminase fusion protein that can convert several amino acids to create a stop codon (e.g., TAA, TAG, or TGA).

In some embodiments, simultaneous introduction of mutations into more than one protein factors in the LDL-mediated cholesterol clearance pathway are provided. For example, in some embodiments, a mutation may be simultaneously introduced into one or more, preferably at least two, of ANGPTL3, PCSK9, LDLR, APOB, APOE, IDOL, and other LDL-mediated pathway involved genes. In some embodiments, a loss-of-function mutation may be simultaneously introduced into one or more, preferably at least two, of ANGPTL3, PCSK9, APOB, and another LDL-mediated pathway involved gene. In some embodiments, mutations may be simultaneously introduced into ANGPTL3, PCSK9, LDLR, and IDOL. To simultaneously introduce of loss-of-function mutations into more than one protein, multiple guide nucleotide sequences are used.

In some embodiments, the target sequence may be located in a non-genic functional site in the genome that controls aspects of chromatin organization, such as a scaffold site or locus control region. In some embodiments, the target sequence may be a genetic safe harbor site, i.e., a locus that facilitates safe genetic modification.

### Templates

In some embodiments, at least one template may be provided as a substrate during the repair of the cleaved target nucleic acid molecule. In some embodiments, the template may be used in homologous recombination, such as, e.g., high-fidelity homologous recombination. In some embodiments, the homologous recombination may result in the integration of the template sequence into the target nucleic acid molecule. In some embodiments, a single template or multiple copies of the same template may be provided. In other embodiments, two or more templates may be provided such that homologous recombination may occur at two or more target sites. For example, different templates may be provided to repair a single gene in a cell, or two different genes in a cell. In some embodiments, the different templates may be provided in independent copy numbers.

In some embodiments, the template may be used in homology-directed repair, requiring DNA strand invasion at the site of the cleavage in the nucleic acid. In some embodiments, the homology-directed repair may result in the copying of the template sequence into the target nucleic acid molecule. In some embodiments, a single template or multiple copies of the same template may be provided. In other embodiments, two or more templates having different sequences may be inserted at two or more sites by homology-directed repair. For example, different templates may be provided to repair a single gene in a cell, or two different genes in a cell. In some embodiments, the different templates may be provided in independent copy numbers.

In some embodiments, the template may be incorporated into the cleaved nucleic acid as an insertion mediated by non-homologous end joining. In some embodiments, the template sequence has no similarity to the nucleic acid sequence near the cleavage site. In some embodiments, the template sequence (e.g., the coding sequence in the template) has no similarity to the nucleic acid sequence near the cleavage site. The template sequence may be flanked by target sequences that may have similar or identical sequence(s) to a target sequence near the cleavage site. In some embodiments, a single template or multiple copies of the same template may be provided. In other embodiments, two or more templates having different sequences may be inserted at two or more sites by non-homologous end joining. For example, different templates may be provided to insert a single template in a cell, or two different templates in a cell. In some embodiments, the different templates may be provided in independent copy numbers.

In some embodiments, the template sequence may correspond to an endogenous sequence of a target cell. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell. In some embodiments, the template sequence may be substantially identical to a portion of the endogenous sequence in a cell at or near the cleavage site, but comprise at least one nucleotide change. In some embodiments, the repair of the cleaved target nucleic acid molecule with the template may result in a mutation comprising an insertion, deletion, or substitution of one or more nucleotides of the target nucleic acid molecule. In some embodiments, the mutation may result in one or more amino acid changes in a protein expressed from a gene comprising the target sequence. In some embodiments, the mutation may result in one or more nucleotide changes in an RNA expressed from the target gene. In some embodiments, the mutation may alter the expression level of the target gene. In some embodiments, the mutation may result in increased or decreased expression of the target gene. In some embodiments, the mutation may result in gene knockdown. In some embodiments, the mutation may result in gene knockout. In some embodiments, the repair of the cleaved target nucleic acid molecule with the template may result in replacement of an exon sequence, an intron sequence, a transcriptional control sequence, a translational control sequence, or a non-coding sequence of the target gene.

In other embodiments, the template sequence may comprise an exogenous sequence. In some embodiments, the exogenous sequence may comprise a protein or RNA coding sequence operably linked to an exogenous promoter sequence such that, upon integration of the exogenous sequence into the target nucleic acid molecule, the cell is capable of expressing the protein or RNA encoded by the integrated sequence. In other embodiments, upon integration of the exogenous sequence into the target nucleic acid molecule, the expression of the integrated sequence may be regulated by an endogenous promoter sequence. In some embodiments, the exogenous sequence may be a chromosomal or extrachromosomal sequence. In some embodiments, the exogenous sequence may provide a cDNA sequence encoding a protein or a portion of the protein. In yet other embodiments, the exogenous sequence may comprise an exon sequence, an intron sequence, a transcriptional control sequence, a translational control sequence, or a non-coding sequence. In some embodiments, the integration of the exogenous sequence may result in gene knock-in.

The template may be of any suitable length. In some embodiments, the template may comprise 10, 15, 20, 25, 50, 75, 100, 150, 200, 500, 1000, 1500, 2000, 2500, 3000, 3500, 4000, 4500, 5000, 5500, 6000, or more nucleotides in length. In some embodiments, the template may comprise a nucleotide sequence that is complementary to a portion of the target nucleic acid molecule comprising the target sequence (i.e., a “homology arm”). In some embodiments, a homology arm may comprise 10, 15, 20, 25, 50, 75, 100, 150, 200, 500, 1000, 1500, 2000, 2500, 3000 or more nucleotides in length. In some embodiments, the template may comprise a homology arm that is complementary to the sequence located upstream or downstream of the cleavage site on the target nucleic acid molecule. In some embodiments, the template may comprise a first nucleotide sequence and a second homology arm that are complementary to the sequences located upstream and downstream of the cleavage site, respectively. Where a template contains two homology arms, each arm can be the same length or different lengths, and the sequence between the homology arms can be substantially similar or identical to the target sequence between the homology arms, or be entirely unrelated. In some embodiments, the degree of complementarity between the first nucleotide sequence on the template and the sequence upstream of the cleavage site, and between the second nucleotide sequence on the template and the sequence downstream of the cleavage site, may permit homologous recombination, such as, e.g., high-fidelity homologous recombination, between the template and the target nucleic acid molecule. In some embodiments, the degree of complementarity may be about 50%, 55%, 60%, 65%, 70%, 75%, 80%, 85%, 90%, 95%, 97%, 98%, 99%, or 100%. In some embodiments, the degree of complementarity may be about 95%, 97%, 98%, 99%, or 100%. In some embodiments, the degree of complementarity may be about 98%, 99%, or 100%. In some embodiments, the degree of complementarity may be 100%. In some embodiments, for example those described herein where a template is incorporated into the cleaved nucleic acid as an insertion mediated by non-homologous end joining, the template has no homology arms. In some embodiments, a template having no homology arms comprises target sequences flanking one or both ends of the template sequence, e.g., as described herein. In some embodiments, a template having no homology arms comprises target sequences flanking both ends of the template sequence. In some embodiments, a target sequence flanking the end of the template sequence is about 10-50 nucleotides. In some embodiments, a target sequence flanking the end of the template sequence is about 10-20 nucleotides, about 15-20 nucleotides, about 20-25 nucleotides, or about 20-30 nucleotides. In some embodiments, a target sequence flanking the end of the template sequence is about 17-23 nucleotides. In some embodiments, a target sequence flanking the end of the template sequence is about 20 nucleotides.

In some embodiments, a nucleic acid molecule is expressed from the template if homologous recombination occurs between the template and the genomic sequence. In some embodiments, for example, the template does not have a promoter for expressing the nucleic acid molecule and/or the ATG transcriptional start site is removed from the coding sequence.

### Delivery

Provided herein are methods and compositions for editing a nucleic acid molecule in a cell with a nuclease system and targeted delivery thereof. In some embodiments, the nucleic acid comprises a nucleic acid sequence encoding a gene. In some embodiments, the nucleic acid comprises a nucleic acid sequence encoding a gene associated with a disease or disorder.

The active agents comprising nucleic acids described herein, e.g. modified guide RNAs, may be conjugated with one or more targeting moieties for targeted delivery to desired in vivo locations. The guide RNA conjugates or guide RNA-protein complex conjugates may be introduced into the cell via any methods known in the art, such as, e.g., viral or bacteriophage infection, transfection, conjugation, protoplast fusion, lipofection, lipid particle or vesicle transduction, electroporation, calcium phosphate precipitation, polyethyleneimine (PEI)-mediated transfection, DEAE-dextran-mediated transfection, liposome-mediated transfection, e.g. transfection mediated by cationic liposomes, particle gun technology, calcium phosphate precipitation, shear-driven cell permeation, fusion to a cell-penetrating peptide followed by cell contact, microinjection, and nanoparticle-mediated delivery. In some embodiments, the nuclease system may be introduced into the cell via viral infection. In some embodiments, the nuclease system may be introduced into the cell via bacteriophage infection. Liposomes may include those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and liposomes which may deliver small molecule drugs such as, but not limited to, DOXIL® (from Janssen Biotech, Inc. (Horsham, PA)).

In some embodiments, the methods and compositions provided herein may comprise introducing a vector system described herein into a cell. In some embodiments, the vector system encodes the nuclease system in whole or in part. In some embodiments, the vector system comprises one, two, three, or more vectors. In some embodiments, the introduction of the vector system into the cell may result in a stable cell line having the edited nucleic acid molecule while the vectors are lost, e.g., targeted for self-destruction. In some embodiments, the cell is a eukaryotic cell. Non-limiting examples of eukaryotic cells include yeast cells, plant cells, insect cells, cells from an invertebrate animal, cells from a vertebrate animal, mammalian cells, rodent cells, mouse cells, rat cells, and human cells. In some embodiments, the eukaryotic cell may be a mammalian cell. In some embodiments, the eukaryotic cell may be a rodent cell. In some embodiments, the eukaryotic cell may be a human cell. Similarly, the target sequence may be from any such cells or in any such cells.

In some embodiments, the polynucleotides or oligonucleotides provided herein, for example guide RNAs or mRNAs, may be formulated in a lipid vesicle which may have crosslinks between functionalized lipid bilayers, or lipid-polycation complex. The liposome formulation may be influenced by, but not limited to, the selection of the cationic lipid component, the degree of cationic lipid saturation, the nature of the PEGylation, ratio of all components and biophysical parameters such as size, or poly cationic composition. In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo. The lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), respiratory (e.g., nasal, pharyngeal, tracheal and bronchial membranes), genital (e.g., vaginal, cervical and urethral membranes). The formulations may use nanoparticles larger than 10-200 nm which are preferred for higher drug encapsulation efficiency and the ability to provide the sustained delivery of a wide array of drugs which had been thought to be too large to rapidly diffuse through mucosal barriers. The dynamic transport of nanoparticles may be measured using fluorescence recovery after photobleaching (FRAP) and high resolution multiple particle tracking (MPT). The formulations can be made for controlled release and/or targeted delivery. The lipid nanoparticle engineered to penetrate mucus may include surface altering agents such as, but not limited to, mRNA, anionic protein (e.g., bovine serum albumin), surfactants (e.g., cationic surfactants such as for example dimethyldioctadecylammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4, domase alfa, neltenexine, erdosteine) and various DNases including rhDNase. The surface altering agent may be embedded or enmeshed in the particle's surface or disposed or dispersed (e.g., by coating, adsorption, covalent linkage, or other process) on the surface of the lipid nanoparticle.

In a further embodiment, guide RNA of the present disclosure and the CRISPR system may be formulated as a lipoplex, such as, without limitation, the ATUPLEX™ system, the DACC system, the DBTC system and other siRNA-lipoplex technology. The liposomes, lipoplexes, or lipid nanoparticles may be used to improve the efficacy of the modified guide RNAs for example by increasing cell transfection, increasing the translation of encoded protein or increasing the stability. A cell penetrating peptide may be used with the pharmaceutical formulations of the present disclosure such as a cell-penetrating peptide sequence attached to polycations that facilitates delivery to the intracellular space, e.g., HIV-derived TAT peptide, penetratins, transportans, or hCT derived cell-penetrating peptides. In another embodiment, lipid nanoparticles which target specific cell types may be used. Alternatively, the lipid nanoparticle may be encapsulated into any polymer or hydrogel known in the art which may form a gel when injected into a subject. As another non-limiting example, the lipid nanoparticle may be encapsulated into a polymer matrix which may be biodegradable. In yet another embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, FL), HYLENEX® (Halozyme Therapeutics, San Diego, CA), surgical sealants such as fibrinogen polymers (Ethicon Inc. Cornelia, GA), TISSELL® (Baxter International, Inc Deerfield, IL), PEG-based sealants, and COSEAL® (Baxter International, Inc Deerfield, IL).

In some embodiments, the nucleic acids as described herein, such as a guide RNA, may be complexed with a CRISPR enzyme. In some embodiments, a part or all of the complex may be delivered via a vector system comprising one or more vectors. In some embodiments, the vector may be a DNA vector. In other embodiments, the vector may be an RNA vector. In some embodiments, the RNA vector may be an mRNA, e.g. an mRNA that encodes a nuclease such as Cas9. In some embodiments, the vector may be circular. In other embodiments, the vector may be linear. Non-limiting exemplary vectors include plasmids, phagemids, cosmids, artificial chromosomes, minichromosomes, transposons, viral vectors, and expression vectors. In some embodiments, the nuclease is provided by an RNA vector, e.g., as mRNA, and the template is provided by a viral vector. In some embodiments, the vector may be a viral vector. In some embodiments, the viral vector may be genetically modified from its wild-type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without any helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, herpes simplex virus (HSV-1) vectors, bacteriophage T4, baculovirus vectors, and retrovirus vectors. In some embodiments, the viral vector may be an AAV vector. In other embodiments, the viral vector may a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (Ψ) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas9 protein, while a second AAV vector may contain one or more guide sequences and one or more copies of template.

In certain embodiments, a viral vector may be modified to target a particular tissue or cell type. For example, viral surface proteins may be altered to decrease or eliminate viral protein binding to its natural cell surface receptor(s). In some embodiments, the vector may be modified for liver specific delivery. The surface proteins may also be engineered to interact with a receptor specific to a desired cell type. Viral vectors may have altered host tropism, including limited or redirected tropism. In some embodiments, the viral vector may be engineered to express or display a first binding moiety. The first binding moiety may be fused to a viral surface protein or glycoprotein, conjugated to a virus, chemically crosslinked to a virion, bound to a virus envelope, or joined to a viral vector by any other suitable method. The first binding moiety is capable of binding to a second binding moiety, which may be used to direct the virus to a desired cell type. In some embodiments, the first binding moiety is avidin, streptavidin, neutravidin, captavidin, or another biotin-binding moiety, and the second binding moiety is biotin or an analog thereof. A biotinylated targeting agent may then be bound to the avidin on the viral vector and used to direct the virus to a desired cell type. For example, a T4 vector may be engineered to display a biotin-binding moiety on one or more of its surface proteins. The cell-specificity of such a T4 vector may then be altered by binding a biotinylated antibody or ligand directed to a cell of choice. In alternate embodiments, the first and second binding moieties are hapten and an anti-hapten binding protein; digoxigenin and an anti-digoxigenin binding protein; fluorescein and an anti-fluorescein binding protein; or any other suitable first and second binding moieties that are binding partners.

In some embodiments, the vector may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding the nuclease described herein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter. In some embodiments, the nucleotide sequence encoding the nuclease may be operably linked to at least one transcriptional or translational control sequence.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1α) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1α promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the vector may encode a Cas protein or a portion of a Cas protein, such as a Cas9 protein or Cpf1 protein. The vector system may further comprise a vector comprising a nucleotide sequence encoding the guide RNA described herein. In some embodiments, the vector system may comprise one copy of the guide RNA. In other embodiments, the vector system may comprise more than one copy of the guide RNA. In embodiments with more than one guide RNA, the guide RNAs may be non-identical such that they target different target sequences, or have other different properties, such as activity or stability within the Cas9 RNP complex. In some embodiments, the nucleotide sequence encoding the guide RNA may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, H1 and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA. In other embodiments, the crRNA and the tracr RNA may be driven by their corresponding promoters on the same vector. In yet other embodiments, the crRNA and the tracr RNA may be encoded by different vectors.

In some embodiments, the vector system may further comprise a vector comprising the template described herein. In some embodiments, the vector system may comprise one copy of the template. In other embodiments, the vector system may comprise more than one copy of the template. In some embodiments, the vector system may comprise 2, 3, 4, 5, 6, 7, 8, 9, 10, or more copies of the template. In some embodiments, the vector system may comprise 4, 5, 6, 7, 8, or more copies of the template. In some embodiments, the vector system may comprise 5, 6, 7, or more copies of the template. In some embodiments, the vector system may comprise 6 copies of the template. The multiple copies of the template may be located on the same or different vectors. The multiple copies of the template may also be adjacent to one another, or separated by other nucleotide sequences or vector elements. In other embodiments, two or more templates may be provided such that homologous recombination may occur at two or more target sites. For example, different templates may be provided to repair a single gene in a cell, or two different genes in a cell. In some embodiments, the different templates may be provided in independent copy numbers.

A vector system may comprise 1-3 vectors. In some embodiments, the vector system may comprise one single vector. In other embodiments, the vector system may comprise two vectors. In additional embodiments, the vector system may comprise three vectors.

In some embodiments, the nucleotide sequence encoding the nuclease and the template may be located on the same or separate vectors. In some embodiments, the nucleotide sequence encoding the nuclease and the template may be located on the same vector. In some embodiments, the nucleotide sequence encoding the nuclease and the template may be located on separate vectors. The sequences may be oriented in the same or different directions and in any order on the vector.

In some embodiments, the nucleotide sequence encoding a Cas9 protein and a template may be located on the same or separate vectors. In some embodiments, all of the sequences may be located on the same vector. In some embodiments, two or more sequences may be located on the same vector. The sequences may be oriented in the same or different directions and in any order on the vector. In some embodiments, the nucleotide sequence encoding the Cas9 protein and the nucleotide sequence encoding the guide RNA may be located on the same vector. In some embodiments, the nucleotide sequence encoding the Cas9 protein and the template may be located on the same vector. In a particular embodiment, the vector system may comprise a first vector comprising the nucleotide sequence encoding the Cas9 protein, and a second vector comprising the nucleotide sequence encoding the template or multiple copies of the template.

In some embodiments, the template may be released from the vector on which it is located by the nuclease system encoded by the vector system. In some embodiments, the template may be released from the vector by a Cas9 protein provided from an mRNA. The template may comprise at least one target sequence that is recognized by the guide RNA. In some embodiments, the template may be flanked by a target sequence at the 5′ and 3′ ends of the template. Upon expression of Cas9 protein and delivery of the guide RNA, the guide RNA may hybridize with and the Cas9 protein may cleave the target sequence at both ends of the template such that the template is released from the vector. In additional embodiments, the template may be released from the vector by a nuclease encoded by the vector system by having a target sequence recognized by the nuclease at the 5′ and 3′ ends of the template. The target sequences at either end of the template may be oriented such that the PAM sequence is closer to the template. In such an orientation, fewer non-template nucleic acids remain on the ends of the template after release from the vector. In some embodiments, the target sequences flanking the template may be the same. In some embodiments, the target sequences flanking the template may be the same as the target sequence found at the cleavage site in which the template is incorporated, e.g., by HR, HDR, or non-homologous end joining. In other embodiments, the target sequences flanking the template may be different. For example, the target sequence at the 5′ end of the template may be recognized by one guide RNA or nuclease, and the target sequence at the 3′ end of the template may be recognized by another guide RNA or nuclease.

In some embodiments, the vector encoding the nuclease system may comprise at least one target sequence within the vector, to create a self-destroying (or “self-cleaving” or “self-inactivating”) vector system to control the amount of the nuclease system to be expressed. In some embodiments, the self-destroying vector system results in a reduction in the amount of nuclease activity. In further embodiments, the self-destroying vector system results in a reduction in the amount of vector nucleic acid. In embodiments in which the system comprises Cas9, it also comprises guide RNA(s) that recognize the target sequence. In this way, the residence time and/or the level of activity of the nuclease system may be temporally controlled to avoid adverse effects associated with overexpression of the nuclease system. Such adverse effects may include, e.g., an off-target effect by the nuclease. In some embodiments, one or more target sequences may be located at any place on the vector such that, upon expression of the nuclease, the nuclease recognizes and cleaves the target sequence in the vector that contains the nuclease-encoding sequence. The one or more target sequences of the self-destroying vector may be the same. Optionally, the self-destroying vector may comprise multiple target sequences. In some embodiments, the cleavage at a target sequence may reduce the expression of at least one component of the nuclease system, such as, for example, Cas9. In some embodiments, the cleavage may reduce the expression of the nuclease transcript. For example, a target sequence may be located within the nucleotide sequence encoding the nuclease such that the cleavage results in the disruption of the coding region. In other embodiments, a target sequence may be located within a non-coding region on the vector encoding the nuclease. In some embodiments, a target sequence may be located within the promoter that drives the expression of the nuclease such that the cleavage results in the disruption of the promoter sequence. For example, the vector may contain a target sequence (and its corresponding guide RNA) that targets a Cas9 sequence. In certain embodiments, a target sequence may be located between the promoter and the nucleotide sequence encoding the nuclease such that the cleavage results in the separation of the coding sequence from its promoter. In certain embodiments, a target sequence outside the nuclease coding sequence and a target sequence within the nuclease coding sequence are included.

In some embodiments, the vector encoding a Cas9 protein may comprise at least one target sequence that is recognized by a guide RNA. In some embodiments, the target sequence may be located at any place on the vector such that, upon expression of the Cas9 protein and the guide RNA, the guide RNA hybridizes with and the Cas9 protein cleaves the target sequence in the vector encoding the Cas9 protein. In some embodiments, the cleavage at the target sequence may reduce the expression of the Cas9 protein transcript. For example, the target sequence may be located within the nucleotide sequence encoding the Cas9 protein such that the cleavage results in the disruption of the coding region. In other embodiments, the target sequence may be located within a non-coding region on the vector encoding the Cas9 protein. In some embodiments, the target sequence may be located within the promoter that drives the expression of the Cas9 protein such that the cleavage results in the disruption of the promoter sequence. In some embodiments, the target sequence may be located within the nucleotide sequence encoding the Cas9 protein such that the cleavage results in the disruption of the coding sequence. In other embodiments, the target sequence may be located between the promoter and the nucleotide sequence encoding the Cas9 protein such that the cleavage results in the separation of the coding sequence from its promoter.

The target sequences for release of the template, for vector self-destruction, and for targeting by the nuclease system in a cell may be the same or different. For example, the target sequence at the 3′ end of the template may be present within the promoter driving the expression of the nuclease (e.g., the Cas9 protein) such that the release of the template simultaneously results in the disruption of the expression of the nuclease (e.g., the Cas9 protein). In some embodiments, both target sequences flanking the template, the target sequences for disrupting the expression of the nuclease (e.g., the Cas9 protein), and the target sequence in the target nucleic acid molecule in a cell may be the same sequence that is recognized by a single guide RNA or nuclease. Thus, in some embodiments, the vector system may comprise only one type of target sequence, and the nuclease system may comprise only one guide RNA. In other embodiments, these target sequences may comprise different sequences that are recognized by different guide RNAs.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue. Non-limiting exemplary tissue-specific promoters include albumin promoter, α-1 antitrypsin promoter, hemopexin promoter, B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter. In particular embodiments, the tissue specific promoter is an albumin promoter, a α-1 antitrypsin promoter, a hepatitis B virus core promoter, or a hemopexin gene promoter. Methods of examining liver specific promoters are described in Kramer et al., Molecular Therapy 7(3): 375-385 (2003), which is incorporated herein in its entirety by reference.

In some embodiments of the present disclosure, the activity of the nuclease system may be temporally regulated by adjusting the residence time, the amount, and/or the activity of the expressed components of the nuclease system. For example, as described herein, the nuclease may be fused with a protein domain that is capable of modifying the intracellular half-life of the nuclease. In certain embodiments involving two or more vectors (e.g., a vector system in which the components described herein are encoded on two or more separate vectors), the activity of the nuclease system may be temporally regulated by controlling the timing in which the vectors are delivered. For example, in some embodiments a vector encoding the nuclease system may deliver the nuclease prior to the vector encoding the template. In other embodiments, the vector encoding the template may deliver the template prior to the vector encoding the nuclease system. In some embodiments, the vectors encoding the nuclease system and template are delivered simultaneously. In certain embodiments, the simultaneously delivered vectors temporally deliver, e.g., the nuclease, template, and/or guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA end. In some embodiments, the PA may be added to the 3′ UTR of the RNA. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP) Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J Virol, 73(4): 2886-92 (1999) and Flajolet et al., J Virol, 72(7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts.

Embodiments of the disclosure also encompass treating a patient with the vector system described herein. In some embodiments, the method may comprise administering the vector system described herein to the patient. The method may be used as a single therapy or in combination with other therapies available in the art. In some embodiments, the patient may have a mutation (such as, e.g., insertion, deletion, substitution, chromosome translocation) in a disease-associated gene. In some embodiments, administration of the vector system may result in a mutation comprising an insertion, deletion, or substitution of one or more nucleotides of the disease-associated gene in the patient. Certain embodiments may include methods of repairing the patient's mutation in the disease-associated gene. In some embodiments, the mutation may result in one or more amino acid changes in a protein expressed from the disease-associated gene. In some embodiments, the mutation may result in one or more nucleotide changes in an RNA expressed from the disease-associated gene. In some embodiments, the mutation may alter the expression level of the disease-associated gene. In some embodiments, the mutation may result in increased or decreased expression of the gene. In some embodiments, the mutation may result in gene knockdown in the patient. In some embodiments, the administration of the vector system may result in the correction of the patient's mutation in the disease-associated gene. In some embodiments, the administration of the vector system may result in gene knockout in the patient. In some embodiments, the administration of the vector system may result in replacement of an exon sequence, an intron sequence, a transcriptional control sequence, a translational control sequence, or a non-coding sequence of the disease-associated gene.

In some embodiments, the administration of the vector system may result in integration of an exogenous sequence of the template into the patient's genomic DNA. In some embodiments, the exogenous sequence may comprise a protein or RNA coding sequence operably linked to an exogenous promoter sequence such that, upon integration of the exogenous sequence into the patient's genomic DNA, the patient is capable of expressing the protein or RNA encoded by the integrated sequence. The exogenous sequence may provide a supplemental or replacement protein coding or non-coding sequence. For example, the administration of the vector system may result in the replacement of the mutant portion of the disease-associated gene in the patient. In some embodiments, the mutant portion may include an exon of the disease-associated gene. In other embodiments, the integration of the exogenous sequence may result in the expression of the integrated sequence from an endogenous promoter sequence present on the patient's genomic DNA. For example, the administration of the vector system may result in supply of a functional gene product of the disease-associated gene to rectify the patient's mutation. In some embodiments, the administration of the vector system may result in integration of a cDNA sequence encoding a protein or a portion of the protein. In yet other embodiments, the administration of the vector system may result in integration of an exon sequence, an intron sequence, a transcriptional control sequence, a translational control sequence, or a non-coding sequence into the patient's genomic DNA. In some embodiments, the administration of the vector system may result in gene knockin in the patient.

### Administration

Provided herein are methods and compositions for editing a target nucleic acid in a cell. Further provided herein are pharmaceutical compositions and methods for modifying the function and activity of a target gene in a cell of a subject. The genome editing compositions described herein may be administered to a subject in need thereof in a therapeutically effective amount, to treat conditions related to high circulating cholesterol levels and/or coronary disease, e.g. hypercholesterolemia, elevated total cholesterol levels, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, and combinations thereof can be administered to the subject in a variety of ways, including parenterally, intravenously, intradermally, intramuscularly, colonically, rectally or intraperitoneally. In some embodiments, the pharmaceutical composition may be co-administered with pharmaceutically acceptable salt by intraperitoneal injection, intramuscular injection, subcutaneous injection, or intravenous injection of the subject. In some embodiments, the pharmaceutical composition may be directly injected to a specific tissue, such as the liver tissue. In some embodiments, the pharmaceutical compositions can be administered parenterally, intravenously, intramuscularly or orally. The oral formulations can be further coated or treated to prevent or reduce dissolution in stomach. The compositions of the present disclosure can be administered to a subject using any suitable methods known in the art. Suitable formulations for use in the present disclosure and methods of delivery are generally well known in the art. For example, the composition of the present disclosure can be formulated as pharmaceutical compositions with a pharmaceutically acceptable diluent, carrier or excipient. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

Pharmaceutical formulations described herein can be administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the pharmaceutical formulation is in the form of a tablet. In other embodiments, pharmaceutical formulations containing an composition or inhibitory agent described herein are in the form of a capsule. In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a composition or inhibitory agent described herein can be formulated for use as an aerosol, a mist or a powder. For buccal or sublingual administration, the compositions may take the form of tablets, lozenges, or gels formulated in a conventional manner. In some embodiments, a composition or inhibitory agent described herein can be prepared as transdermal dosage forms. In some embodiments, a composition or inhibitory agent described herein can be formulated into a pharmaceutical composition suitable for intramuscular, subcutaneous, or intravenous injection. In some embodiments, a composition or inhibitory agent described herein can be administered topically and can be formulated into a variety of topically administrable compositions, such as solutions, suspensions, lotions, gels, pastes, medicated sticks, balms, creams or ointments. In some embodiments, a composition or inhibitory agent described herein can be formulated in rectal compositions such as enemas, rectal gels, rectal foams, rectal aerosols, suppositories, jelly suppositories, or retention enemas.

### Dosage

Appropriate dosage or effective amounts for administration vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. Factors involved in dosage determination are known to those of ordinary skill in the art without additional experimentation other than routine test. It is generally preferred that a maximum dose of the individual components or combinations thereof be used, that is, the highest safe dose according to sound medical judgment. Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen can vary over time. In some embodiments, for an adult subject of normal weight, doses ranging from about 0.01 to 1000 mg/kg may be administered. In some embodiments, the dose is between 1 to 200 mg. In some embodiments, the doses may range from about 0.01 to 0.05 mg/kg, between about 0.01 to 0.1 mg/kg, between about 0.01 to 1 mg/kg, between about 0.01 to 10 mg/kg, between about 0.01 to 100 mg/kg, between 0.01 to 500 mg/kg, between about 0.1 to 1 mg/kg, between about 0.1 to 5 mg/kg, between about 0.1 to 10 mg/kg, between about 0.1 to 100 mg/kg, between about 0.1 to 500 mg/kg, between about 0.1 to 1000 mg/kg, between about 1 to 5 mg/kg, between about 1 to 10 mg/kg, between about 1 to 100 mg/kg, between about 1 to 500 mg/kg, between about 1 to 1000 mg/kg, between about 10 to 100 mg/kg, between about 10 to 500 mg/kg, between about 10 to 1000 mg/kg, or between about 100 to 1000 mg/kg. The particular dosage regimen, i.e., dose, timing and repetition, will depend on the particular subject and that subject's medical history, as well as the properties of the polypeptide or the polynucleotide (such as the half-life of the polypeptide or the polynucleotide, and other considerations well known in the art).

As will be apparent to those skilled in the art, the appropriate dosage of a therapeutic agent as described herein will depend on the specific agent (or compositions thereof) employed, the formulation and route of administration, the type and severity of the disease, whether the polypeptide or the polynucleotide is administered for preventive or therapeutic purposes, previous therapy, the subject's clinical history and response to the antagonist, and the discretion of the attending physician. Typically the clinician will administer a polypeptide until a dosage is reached that achieves the desired result.

Administration of one or more therapeutic compositions, e.g. polypeptides, polynucleotides, or RNPs, can be continuous or intermittent, depending, for example, upon the recipient's physiological condition, whether the purpose of the administration is therapeutic or prophylactic, and other factors known to skilled practitioners. The administration of a polypeptide may be essentially continuous over a preselected period of time or may be in a series of spaced dose, e.g., either before, during, or after developing a disease.

### Biological Samples

A sample, e.g., a biological sample can be taken from a subject. A biological sample can comprise a plurality of biological samples. The plurality of biological samples can contain two or more biological samples; for examples, about 2-1000, 2-500, 2-250, 2-100, 2-75, 2-50, 2-25, 2-10, 10-1000, 10-500, 10-250, 10-100, 10-75, 10-50, 10-25, 25-1000, 25-500, 25-250, 25-100, 25-75, 25-50, 50-1000, 50-500, 50-250, 50-100, 50-75, 60-70, 100-1000, 100-500, 100-250, 250-1000, 250-500, 500-1000, 2, 3, 4, 5, 6, 7, 8, 9, 10, 11, 12, 13, 14, 15, 16, 17, 18, 19, 20, 21, 22, 23, 24, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 90, 95, 100, 110, 120, 130, 140, 150, 160, 170, 180, 190, 200, 210, 220, 230, 240, 250, 275, 300, 325, 350, 375, 400, 425, 450, 475, 500, 550, 600, 650, 700, 750, 800, 850, 900, 950, 1000, or more biological samples. The biological samples can be obtained from a plurality of subjects, giving a plurality of sets of a plurality of samples. The biological samples can be obtained from about 2 to about 1000 subjects, or more; for example, about 2-1000, 2-500, 2-250, 2-100, 2-50, 2-25, 2-20, 2-10, 10-1000, 10-500, 10-250, 10-100, 10-50, 10-25, 10-20, 15-20, 25-1000, 25-500, 25-250, 25-100, 25-50, 50-1000, 50-500, 50-250, 50-100, 100-1000, 100-500, 100-250, 250-1000, 250-500, 500-1000, or at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 68, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, 550, 600, 650, 700, 750, 800, 850, 900, 950, or 1000 or more subjects.

The biological samples can be obtained from human subjects. The biological samples can be obtained from human subjects at different ages. The human subject can be prenatal (e.g., a fetus), a child (e.g., a neonate, an infant, a toddler, a preadolescent), an adolescent, a pubescent, or an adult (e.g., an early adult, a middle aged adult, a senior citizen). The human subject can be between about 0 months and about 120 years old, or older. The human subject can be between about 0 and about 12 months old; for example, about 1, 2, 3, 4, 5, 6, 7, 8, 9, 10, 11, or 12 months old. The human subject can be between about 0 and 12 years old; for example, between about 0 and 30 days old; between about 1 month and 12 months old; between about 1 year and 3 years old; between about 4 years and 5 years old; between about 4 years and 12 years old; about 1, 2, 3, 4, 5, 6, 7, 8, 9, 10, 11, or 12 years old. The human subject can be between about 13 years and 19 years old; for example, about 13, 14, 15, 16, 17, 18, or 19 years old. The human subject can be between about 20 and about 39 year old; for example, about 20, 21, 22, 23, 24, 25, 26, 27, 28, 29, 30, 31, 32, 33, 34, 35, 36, 37, 38, or 39 years old. The human subject can be between about 40 to about 59 years old; for example, about 40, 41, 42, 43, 44, 45, 46, 47, 48, 49, 50, 51, 52, 53, 54, 55, 56, 57, 58, or 59 years old. The human subject can be greater than 59 years old; for example, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 years old. The human subjects can include living subjects or deceased subjects. The human subjects can include male subjects and/or female subjects.

Biological samples can be obtained from any suitable source that allows determination of expression levels of genes, e.g., from cells, tissues, bodily fluids or secretions, or a gene expression product derived therefrom (e.g., nucleic acids, such as DNA or RNA; polypeptides, such as protein or protein fragments). The nature of the biological sample can depend upon the nature of the subject. If a biological sample is from a subject that is a unicellular organism or a multicellular organism with undifferentiated tissue, the biological sample can comprise cells, such as a sample of a cell culture, an excision of the organism, or the entire organism. If a biological sample is from a multicellular organism, the biological sample can be a tissue sample, a fluid sample, or a secretion.

The biological samples can be obtained from different tissues. The term tissue is meant to include ensembles of cells that are of a common developmental origin and have similar or identical function. The term tissue is also meant to encompass organs, which can be a functional grouping and organization of cells that can have different origins. The biological sample can be obtained from any tissue.

The biological samples can be obtained from different tissue samples from one or more humans or non-human animals. Suitable tissues can include connective tissues, muscle tissues, nervous tissues, epithelial tissues or a portion or combination thereof. Suitable tissues can also include all or a portion of a lung, a heart, a blood vessel (e.g., artery, vein, capillary), a salivary gland, a esophagus, a stomach, a liver, a gallbladder, a pancreas, a colon, a rectum, an anus, a hypothalamus, a pituitary gland, a pineal gland, a thyroid, a parathyroid, an adrenal gland, a kidney, a ureter, a bladder, a urethra, a lymph node, a tonsil, an adenoid, a thymus, a spleen, skin, muscle, a brain, a spinal cord, a nerve, an ovary, a fallopian tube, a uterus, vaginal tissue, a mammary gland, a testicle, a vas deferens, a seminal vesicle, a prostate, penile tissue, a pharynx, a larynx, a trachea, a bronchi, a diaphragm, bone marrow, a hair follicle, or a combination thereof. A biological sample from a human or non-human animal can also include a bodily fluid, secretion, or excretion; for example, a biological sample can be a sample of aqueous humour, vitreous humour, bile, blood, blood serum, breast milk, cerebrospinal fluid, endolymph, perilymph, female ejaculate, amniotic fluid, gastric juice, menses, mucus, peritoneal fluid, pleural fluid, saliva, sebum, semen, sweat, tears, vaginal secretion, vomit, urine, feces, or a combination thereof. The biological sample can be from healthy tissue, diseased tissue, tissue suspected of being diseased, or a combination thereof.

In some embodiments, the biological sample is a fluid sample, for example a sample of blood, serum, sputum, urine, semen, or other biological fluid. In certain embodiments the sample is a blood sample. In some embodiments the biological sample is a tissue sample, such as a tissue sample taken to determine the presence or absence of disease in the tissue. In certain embodiments the sample is a sample of thyroid tissue.

The biological samples can be obtained from subjects in different stages of disease progression or different conditions. Different stages of disease progression or different conditions can include healthy, at the onset of primary symptom, at the onset of secondary symptom, at the onset of tertiary symptom, during the course of primary symptom, during the course of secondary symptom, during the course of tertiary symptom, at the end of the primary symptom, at the end of the secondary symptom, at the end of tertiary symptom, after the end of the primary symptom, after the end of the secondary symptom, after the end of the tertiary symptom, or a combination thereof. Different stages of disease progression can be a period of time after being diagnosed or suspected to have a disease; for example, at least about, or at least, 1, 2, 3, 4, 5, 6, 7, 8, 9, 10, 11, 12, 13, 14, 15, 16, 17, 18, 19, 20, 21, 22, 23 or 24 hours; 1, 2, 3, 4, 5, 6, 7, 8, 9, 10, 11, 12, 13, 14, 15, 16, 17, 18, 19, 20, 21, 22, 23, 24, 25, 26, 27 or 28 days; 1, 2, 3, 4, 5, 6, 7, 8, 9, 10, 11, 12, 13, 14, 15, 16, 17, 18, 19 or 20 weeks; 1, 2, 3, 4, 5, 6, 7, 8, 9, 10, 11 or 12 months; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 years after being diagnosed or suspected to have a disease. Different stages of disease progression or different conditions can include before, during or after an action or state; for example, treatment with drugs, treatment with a surgery, treatment with a procedure, performance of a standard of care procedure, resting, sleeping, eating, fasting, walking, running, performing a cognitive task, sexual activity, thinking, jumping, urinating, relaxing, being immobilized, being emotionally traumatized, being shock, and the like.

The methods of the present disclosure provide for analysis of a biological sample from a subject or a set of subjects. The subject(s) may be, e.g., any animal (e.g., a mammal), including but not limited to humans, non-human primates, rodents, dogs, cats, pigs, fish, and the like. The present methods and compositions can apply to biological samples from humans, as described herein.

A biological sample can be obtained by methods known in the art such as the biopsy methods provided herein, swabbing, scraping, phlebotomy, or any other suitable method. The biological sample can be obtained, stored, or transported using components of a kit of the present disclosure. In some cases, multiple biological samples, such as multiple thyroid samples, can be obtained for analysis, characterization, or diagnosis according to the methods of the present disclosure. In some cases, multiple biological samples, such as one or more samples from one tissue type (e.g., thyroid) and one or more samples from another tissue type (e.g., buccal) can be obtained for diagnosis or characterization by the methods of the present disclosure. In some cases, multiple samples, such as one or more samples from one tissue type (e.g., thyroid) and one or more samples from another tissue (e.g., buccal) can be obtained at the same or different times. In some cases, the samples obtained at different times are stored and/or analyzed by different methods. For example, a sample can be obtained and analyzed by cytological analysis (e.g., using routine staining). In some cases, a further sample can be obtained from a subject based on the results of a cytological analysis. The diagnosis of a disease or condition, e.g. a coronary disease can include examination of a subject by a physician, nurse or other medical professional. The examination can be part of a routine examination, or the examination can be due to a specific complaint including, but not limited to, one of the following: pain, illness, anticipation of illness, presence of a suspicious lump or mass, a disease, or a condition. The subject may or may not be aware of the disease or condition. The medical professional can obtain a biological sample for testing. In some cases the medical professional can refer the subject to a testing center or laboratory for submission of the biological sample. The methods of obtaining provided herein include methods of biopsy including fine needle aspiration, core needle biopsy, vacuum assisted biopsy, incisional biopsy, excisional biopsy, punch biopsy, shave biopsy or skin biopsy. In some cases, the methods and compositions provided herein are applied to data only from biological samples obtained by FNA. In some cases, the methods and compositions provided herein are applied to data only from biological samples obtained by FNA or surgical biopsy. In some cases, the methods and compositions provided herein are applied to data only from biological samples obtained by surgical biopsy. A biological sample can be obtained by non-invasive methods, such methods including, but not limited to: scraping of the skin or cervix, swabbing of the cheek, saliva collection, urine collection, feces collection, collection of menses, tears, or semen. The biological sample can be obtained by an invasive procedure, such procedures including, but not limited to: biopsy, alveolar or pulmonary lavage, needle aspiration, or phlebotomy. The method of biopsy can further include incisional biopsy, excisional biopsy, punch biopsy, shave biopsy, or skin biopsy. The method of needle aspiration can further include fine needle aspiration, core needle biopsy, vacuum assisted biopsy, or large core biopsy. Multiple biological samples can be obtained by the methods herein to ensure a sufficient amount of biological material. Generic methods for obtaining biological samples are also known in the art and further described in for example Ramzy, Ibrahim Clinical Cytopathology and Aspiration Biopsy 2001 which is herein incorporated by reference in its entirety. The biological sample can be a fine needle aspirate of a thyroid nodule or a suspected thyroid tumor. The fine needle aspirate sampling procedure can be guided by the use of an ultrasound, X-ray, or other imaging device.

In some cases, the subject can be referred to a specialist such as an oncologist, surgeon, or endocrinologist for further diagnosis. The specialist can likewise obtain a biological sample for testing or refer the individual to a testing center or laboratory for submission of the biological sample. In any case, the biological sample can be obtained by a physician, nurse, or other medical professional such as a medical technician, endocrinologist, cytologist, phlebotomist, radiologist, or a pulmonologist. The medical professional can indicate the appropriate test or assay to perform on the sample, or the molecular profiling business of the present disclosure can consult on which assays or tests are most appropriately indicated. The molecular profiling business can bill the individual or medical or insurance provider thereof for consulting work, for sample acquisition and or storage, for materials, or for all products and services rendered.

A medical professional need not be involved in the initial diagnosis or sample acquisition. An individual can alternatively obtain a sample through the use of an over the counter kit. The kit can contain a means for obtaining said sample as described herein, a means for storing the sample for inspection, and instructions for proper use of the kit. In some cases, molecular profiling services are included in the price for purchase of the kit. In other cases, the molecular profiling services are billed separately.

A biological sample suitable for use by the molecular profiling business can be any material containing tissues, cells, nucleic acids, genes, gene fragments, expression products, gene expression products, and/or gene expression product fragments of an individual to be tested. Methods for determining sample suitability and/or adequacy are provided. The biological sample can include, but is not limited to, tissue, cells, and/or biological material from cells or derived from cells of an individual. The sample can be a heterogeneous or homogeneous population of cells or tissues. The biological sample can be obtained using any method known to the art that can provide a sample suitable for the analytical methods described herein.

Obtaining a biological sample can be aided by the use of a kit. A kit can be provided containing materials for obtaining, storing, and/or shipping biological samples. The kit can contain, for example, materials and/or instruments for the collection of the biological sample (e.g., sterile swabs, sterile cotton, disinfectant, needles, syringes, scalpels, anesthetic swabs, knives, curette blade, liquid nitrogen, etc.). The kit can contain, for example, materials and/or instruments for the storage and/or preservation of biological samples (e.g., containers; materials for temperature control such as ice, ice packs, cold packs, dry ice, liquid nitrogen; chemical preservatives or buffers such as formaldehyde, formalin, paraformaldehyde, glutaraldehyde, alcohols such as ethanol or methanol, acetone, acetic acid, HOPE fixative (Hepes-glutamic acid buffer-mediated organic solvent protection effect), heparin, saline, phosphate buffered saline, TAPS, bicine, Tris, tricine, TAPSO, HEPES, TES, MOPS, PIPES, cadodylate, SSC, MES, phosphate buffer; protease inhibitors such as aprotinin, bestatin, calpain inhibitor I and II, chymostatin, E-64, leupeptin, alpha-2-macroglobulin, pefabloc SC, pepstatin, phenylmethanesufonyl fluoride, trypsin inhibitors; DNAse inhibitors such as 2-mercaptoethanol, 2-nitro-5-thicyanobenzoic acid, calcium, EGTA, EDTA, sodium dodecyl sulfate, iodoacetate, etc.; RNAse inhibitors such as ribonuclease inhibitor protein; double-distilled water; DEPC (diethyprocarbonate) treated water, etc.). The kit can contain instructions for use. The kit can be provided as, or contain, a suitable container for shipping. The shipping container can be an insulated container. The shipping container can be self-addressed to a collection agent (e.g., laboratory, medical center, genetic testing company, etc.). The kit can be provided to a subject for home use or use by a medical professional. Alternatively, the kit can be provided directly to a medical professional.

One or more biological samples can be obtained from a given subject. In some cases, between about 1 and about 50 biological samples are obtained from the given subject; for example, about 1-50, 1-40, 1-30, 1-25, 1-20, 1-15, 1-10, 1-7, 1-5, 5-50, 5-40, 5-30, 5-25, 5-15, 5-10, 10-50, 10-40, 10-25, 10-20, 25-50, 25-40, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 biological samples can be obtained from the given subject. Multiple biological samples from the given subject can be obtained from the same source (e.g., the same tissue), e.g., multiple blood samples, or multiple tissue samples, or from multiple sources (e.g., multiple tissues). Multiple biological samples from the given subject can be obtained at the same time or at different times. Multiple biological samples from the given subject can be obtained at the same condition or different condition. Multiple biological samples from the given subject can be obtained at the same disease progression or different disease progression of the subject. If multiple biological samples are collected from the same source (e.g., the same tissue) from the particular subject, the samples can be combined into a single sample. Combining samples in this way can ensure that enough material is obtained for testing and/or analysis.

Also described herein is an RNA molecule comprising the IVT mRNA sequence initiator described herein. In some embodiments, the RNA comprises a guide RNA or a nuclease mRNA.

Also described herein is a cell containing an RNA molecule comprising the IVT mRNA sequence initiator described herein.

Also described herein is a cell containing a polypeptide translated from an RNA molecule comprising the IVT mRNA sequence initiator described herein.

Also described herein is a method for synthesizing an RNA molecule comprising:
a. introducing the IVT mRNA sequence initiator described herein into a mixture comprising an RNA polymerase, and
b. incubating the mixture for a time sufficient to allow for transcription of the RNA molecule.

Also described herein is a method of gene editing comprising introducing into a cell an RNA molecule described herein, or pharmaceutical composition described herein, wherein the RNA molecule comprises guide RNA or a nuclease mRNA, wherein the RNA molecule is translated in the cell.

### EXAMPLES

The following examples are provided to better illustrate the present disclosure and are not to be interpreted as limiting the scope of the disclosure. To the extent that specific materials are mentioned, it is merely for purposes of illustration and is not intended to limit the disclosure. One skilled in the art may develop equivalent means or reactants without the exercise of inventive capacity and without departing from the scope of the disclosure.

### Synthesis of Cap Structures

### Example 1. Preparation of Trinucleotide Caps 1007a, 1032a, 1037a, 1107a, 1112a, 1137a, 1141a, 3102a, 3107a, 3207a, 3212a, 1007c, 1112c, 3102c, 3107c, 3207c, and 3212c

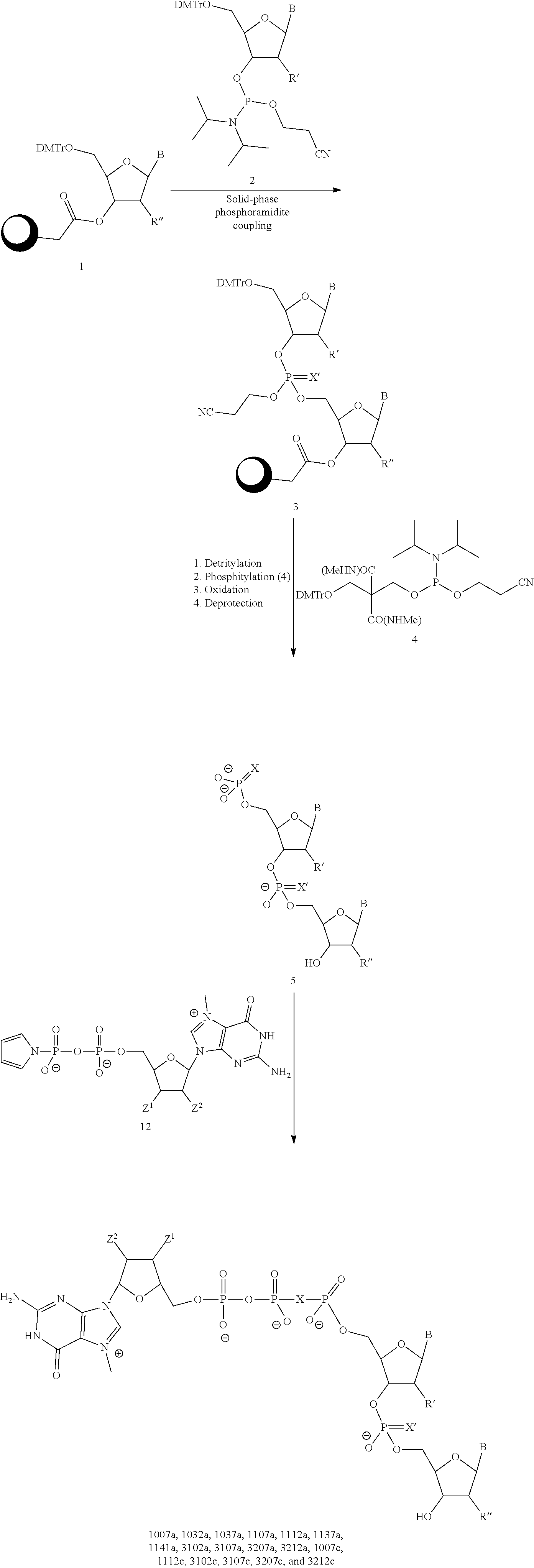

The dinucleotide 5′-phosphate 7 is prepared from the solid support 1 and phosphoramidite 3 by following standard solid phase oligonucleotide synthesis and deprotection protocol (Methods in Molecular Biology, 1993, 20, 81-114; ACS Chem. Biol. 2015, 10, 1181-1187, incorporated herein by reference in its entirety). The phosphoramidite 2 are purchased from commercial sources and/or custom synthesized. The detritylation of support bound fully protected dinucleotide 3 affords the 5′-deprotected dinucleotide intermediate on the support. The intermediate thus obtained is reared the 5′-phosphorylating agent 4 according to reported procedures (EP 816368 A1, https://www.glenresearch.com/solid-chemical-phosphorylation-reagent-ii.html, The Glen Report, 2011, 23, 10-11). After coupling and oxidation, the 5′-phosphorylated dinucleotide is treated with base followed by HPLC purification to obtain the fully deprotected dinucleotide 5′-phosphate 5 (https://www.glenresearch.com/solid-chemical-phosphorylation-reagent-ii.html).

Compound 12 is prepared from guanosine or 2′ and/or 3′ sugar-modified guanosine according to reported procedure (Nucleic Acids Research, 2016, 44, 9578; RNA, 2001, 7, 1486; WO 2017/053297 A1). Compound 12 is coupled with the dinucleotide 5′-monophosphate 5 as described in the literature (Nucleic Acids Research, 2016, 44, 9578, WO 2017/053297 A1) to afford the desired compounds 1007a, 1032a, 1037a, 1107a, 1112a, 1137a, 1141a, 3102a, 3107a, 3207a, 3212a, 1007c, 1112c, 3102c, 3107c, 3207c, and 3212c.

As generally described here in Example 1, compounds 1007a and 1107a (FIG. 1) were synthesized. A final amount of about 1 gram of compound 1007a was produced; this material was characterized by mass spectrometry and HPLC to have an actual mass of 1161 daltons and purity of 98.4%. Additionally, a final amount of about 250 milligrams of compound 1107a was produced; this material was characterized by mass spectrometry and HPLC to have an actual mass of 1161 daltons and purity of 97.5%. These compounds were used as mRNA initiators, as described in Example 5.

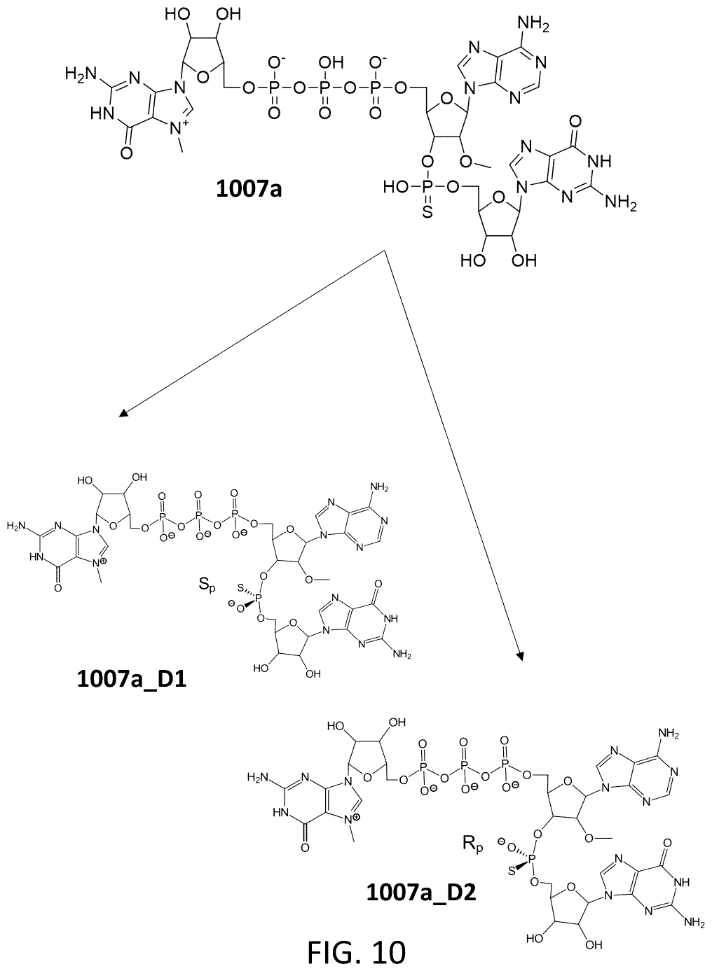

As shown in FIG. 10, the structure of 1007a possesses a sulfur atom bonded to a chiral phosphate. The intrinsic and extrinsic stability of the trimeric compound can be stabilized by the introduction of the sulfur atom at this position. The position of the sulfur atom introduces chirality to the bonded phosphate that did not exist in structure 1002a. The nature of the chirality can produce two chemically identical, but spatially distinct, forms of the structure. The spatial orientation of atoms can change the potency of the desired end function of the chemical compounds. A racemic mixture of 1007a can be purified to separate two distinct forms from the racemic mixture, 1007a_D1 and 1007a_D2. 1007a_D1 (Sp) and 1007a_D2 (Rp) dictate the expected orientation of the sulphur atom around the phosphate.

Production of 1007a_D1 and 1007a_D2 caps may employ any suitable synthesis purification scheme, such as disclosed in the present Example, or Example 13, wherein X═S. Separation/purification of the diastereomers may be achieved through any suitable purification process, including RP-HPLC. Alternatively, it should be understood that the diastereomers might be synthesized separately such that they are stereopure thereby potentially mitigating the need for purification to separate the diastereomers. Such synthesis processes are known in the art. Examples of such synthesis processes can be any process described in Zhang H-J, et al. ((2022) Practical Stereocontrolled Access to Thioisosteres of Essential Signaling Molecules and Building Blocks for Life: Nucleoside Di- and Triphosphates. ChemRxiv. doi:10.26434/chemrxiv-2022-r4hpx).

### Example 2. Preparation of Trinucleotide Caps 1002e and 1007e

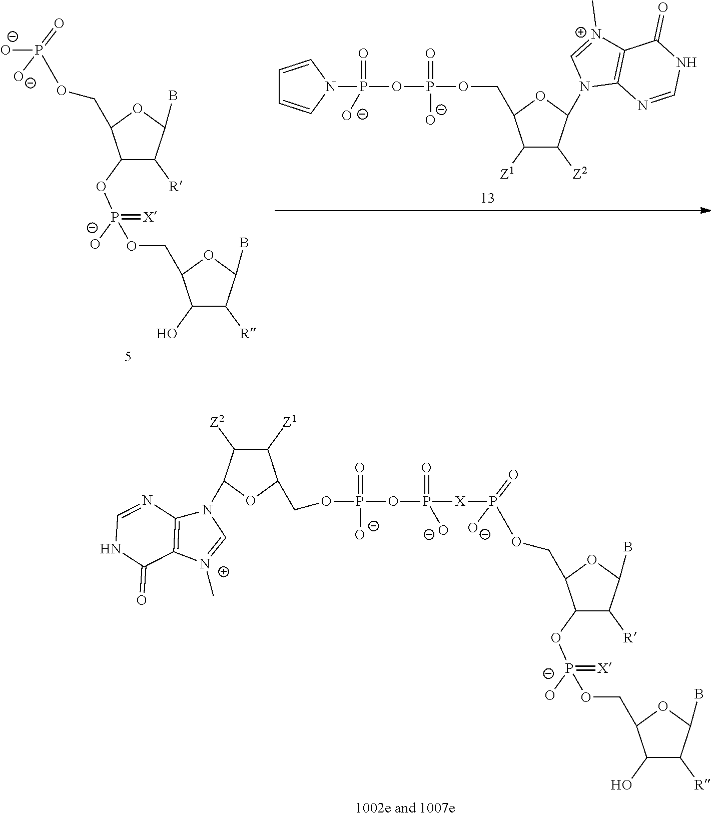

Compound 13 is prepared from inosine or 2′ and/or 3′ sugar-modified inosine (Nucleic Acids Research, 2016, 44, 9578; RNA, 2001, 7, 1486; WO 2017/053297 A1). Compound 13 is then coupled with dinucleotide 5′-monophosphate 5 to afford compounds 1002e and 1007e as described above in Example 1.

### Example 3. Preparation of Trinucleotide Caps 1002i and 1007i

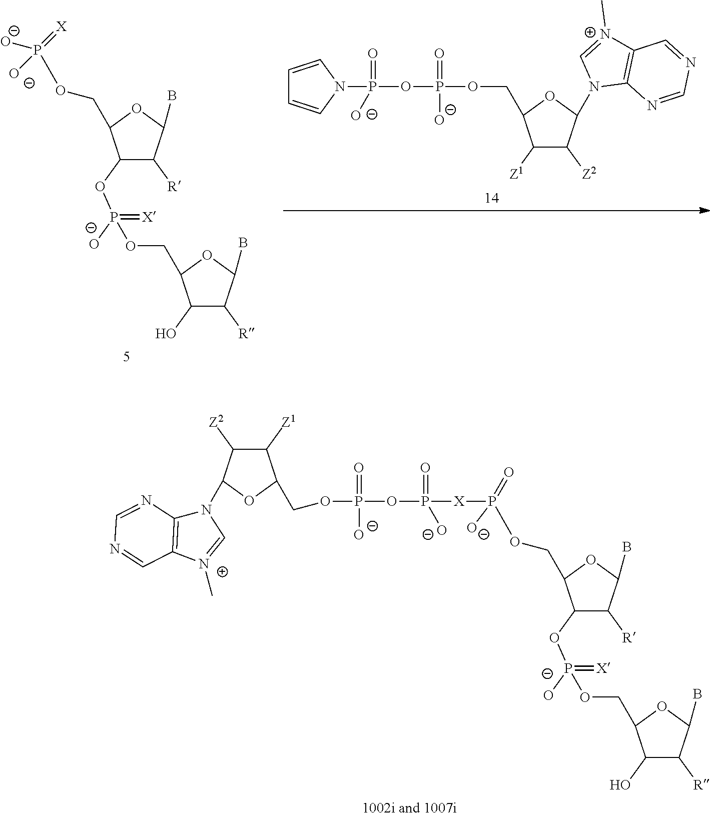

Compound 14 is prepared from nebularine or 2′ and/or 3′ sugar-modified nebularine. Compound 13 is then coupled with dinucleotide 5′-monophosphate 5 to afford compounds 1002i and 1007i as described above in Example 1.

### Example 4. Preparation of Tetranucleotide Caps 5203, 5204, 5207, 5208, 5209, 5210, and 5211

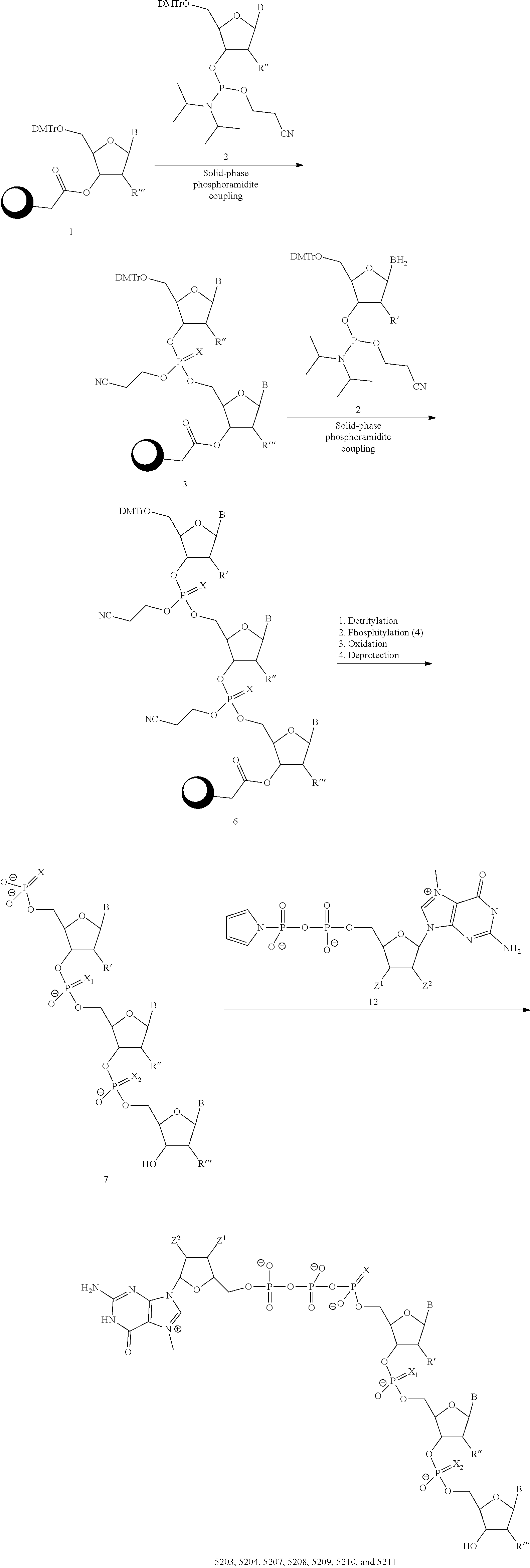

The trinucleotide 5′-monophosphate 7 is prepared form the solid support 1 by following a similar procedure described in Example 1 for the synthesis of the dinucleotide 5′-monophosphate. An additional phosphoramidite is coupled to the 5′-end of the fully-protected solid support bound dinucleotide under standard solid phase oligonucleotide conditions prior to reacting with the 5′-phosphorylating agent 4. Compound 7 thus obtained is then reacted with compound 12 to afford compounds 5203, 5204, 5207, 5208, 5209, 5210, and 5211.

### Example 5. Preparation of Tetranucleotide Caps 5201 and 5205

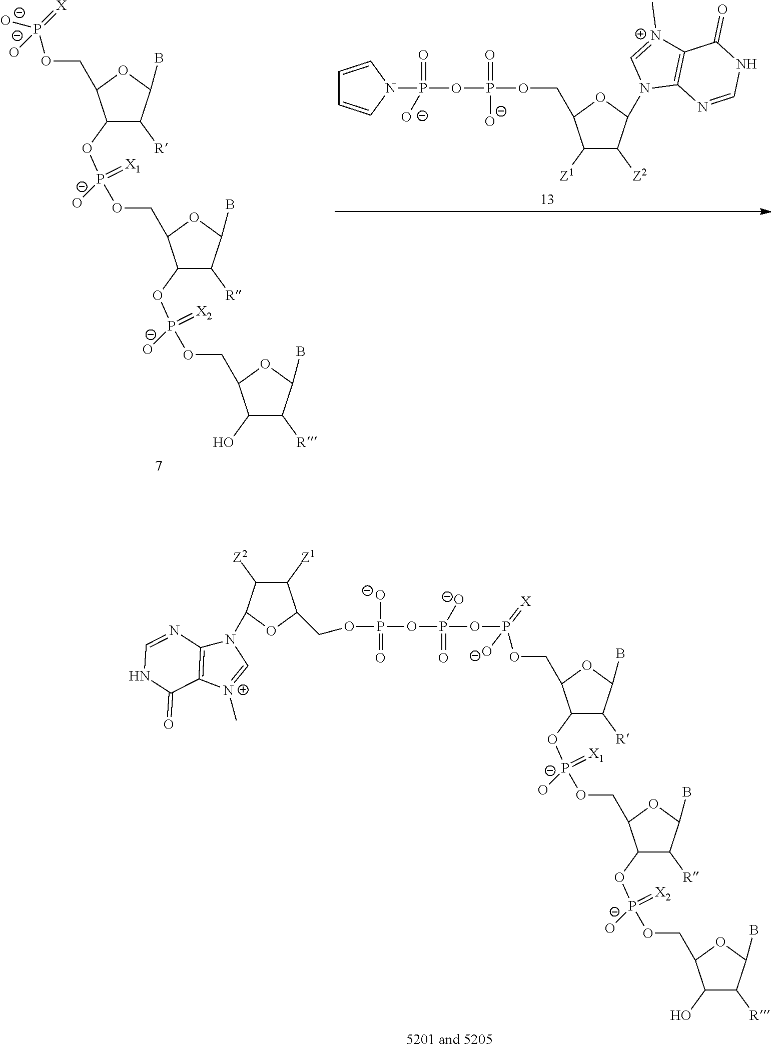

The desired compounds 5201 and 5205 are prepared from compound 7 and compound 13 as described in Examples 1-4.

### Example 6. Preparation of Tetranucleotide Caps 5202 and 5206

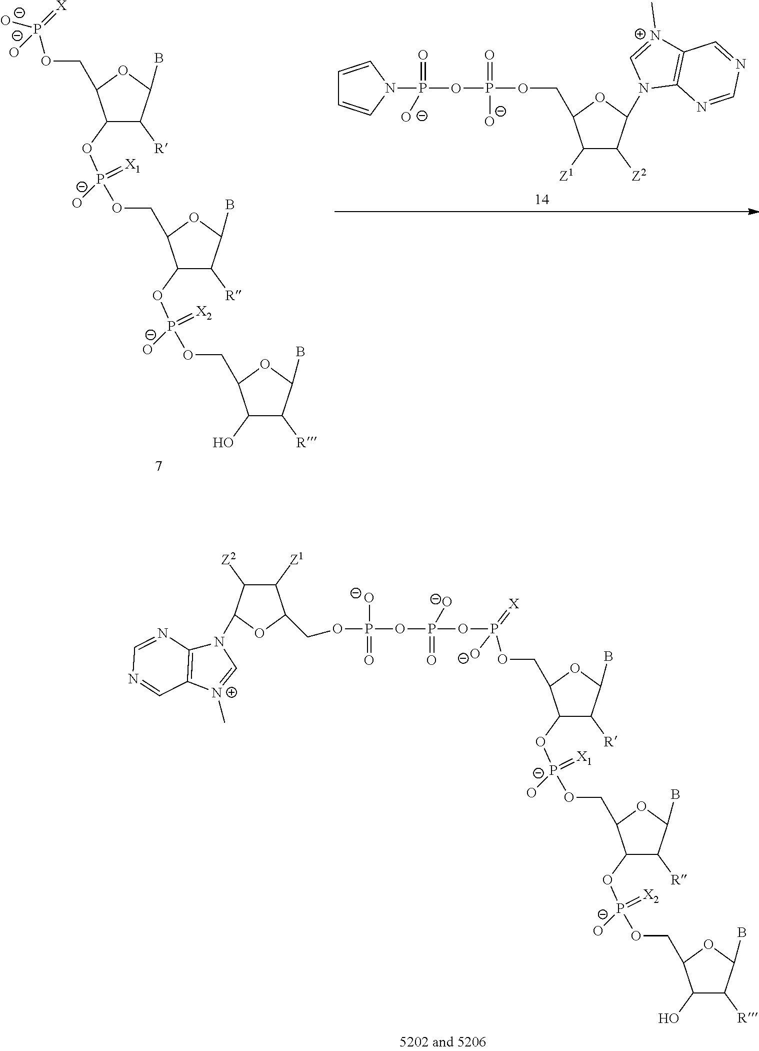

The desired compounds 5202 and 5206 are prepared from compound 7 and compound 14 as described in Examples 1-4.

### Example 7. One-Pot Preparation of 5′ Capped mRNA

A. mRNA Production

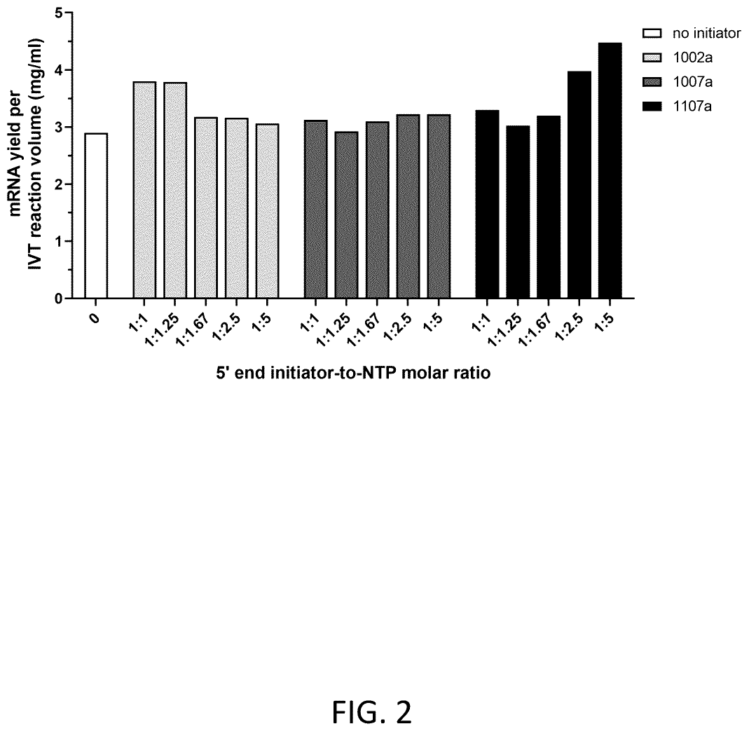

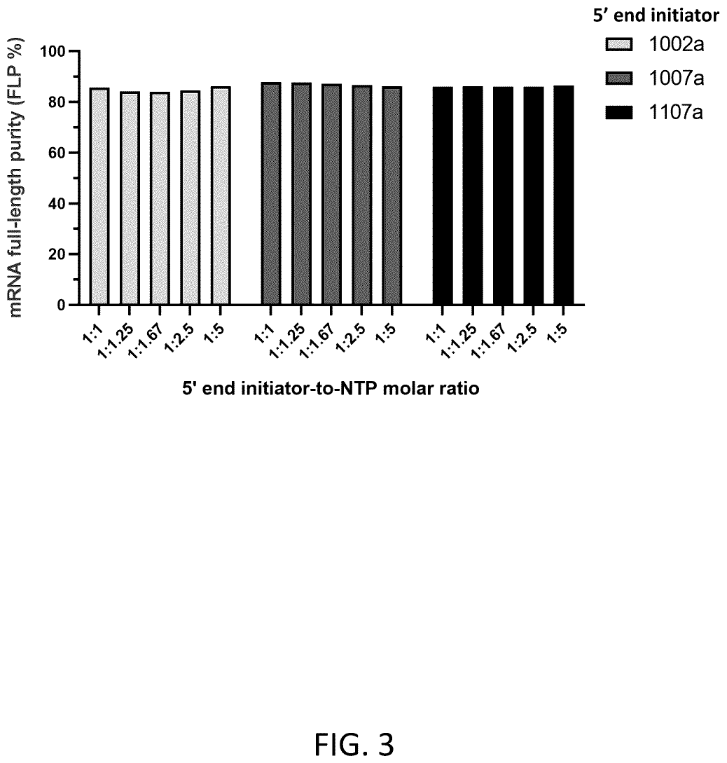

The 5′ end initiator, 1007a, (FIG. 1A) was used to prepare capped mRNAs that encode for ABE and luciferase proteins. In this Example and for all others described herein, the ABE mRNA sequence was MA004 (SEQ ID NO: 3) and the luciferase mRNA sequence was the Photinus pyralis (Firefly) luciferase sequence described in NCBI reference sequence XM_031473197.1. These mRNA can be produced by different methods well known in the art. One of such methods used herein was in vitro transcription (IVT) using T7 polymerase; or in other embodiments, additional RNA polymerase variants. This IVT reaction to produce mRNA used a linearized DNA template that comprised a T7 polymerase promoter and associated regulatory sequences, mRNA coding sequence (CDS), 3′ and 5′ untranslated regions (UTRs), poly A tail, and additional sequence elements to facilitate and modulate mRNA stability and in vivo performance. Prior to IVT, the DNA template was in the form of a plasmid; in other embodiments the DNA template is in the form of a PCR product, synthetic DNA product, or any other double-stranded DNA construct. Linearization of the DNA template with a restriction digestion enzyme was performed in order to promote run-off transcription. The typical IVT reaction included T7 polymerase, DNA template, RNase inhibitor, cap analog, inorganic pyrophosphatase, and the naturally occurring ribonucleoside triphosphates (NTPs) such as GTP, ATP, CTP, and N1-methyl pseudouridine triphosphosphate; in some embodiments NTPs include UTP or modified NTPs such as pseudouridine, 5′methyl cytidine, 5-methoxy uridine, N6-methyl adenosine, and N4-acetyl cytidine. The 1007a 5′ end initiator was added to the IVT reaction along with ATP, GTP, CTP, and N1-methyl pseudouridine triphosphate at a 1:1 initiator-to-NTP molar ratio and additional ratios described in Example 5(B). Poly A tail was encoded in the DNA template that was added to the mRNA during transcription; in some instances, poly A tail is added to the mRNA after the IVT reaction using an RNA ligase enzyme. Finally, T7 RNA polymerase was added to the mixture and the IVT reaction was run at 37° C. for about 1-4 hours. After IVT, DNase was added to the transcription mixture to remove DNA template; in some instances, residual DNA is removed with chromatography. This mRNA was purified to remove residual reaction products and impurities, and stored in 1 mM citrate buffer. Characterization of production yield and mRNA full-length purity was performed by UV spectrometry and capillary gel electrophoresis methods, which are known in the art. The final production yield for ABE encoding mRNA with 5′ end region motif, 1007a, was about 3.3 mg per 1 ml of IVT reaction (FIG. 2) and the mRNA had a full-length purity of about 88% (FIG. 3). As a comparison, the production yield for ABE encoding mRNA with 5′ end motif, 1002a, a commercially available capping compound known in the art, was about 3.8 mg per 1 ml of IVT reaction (FIG. 2) and the mRNA had a full-length purity of about 86% (FIG. 3). Similar IVT and purification process are used to produce mRNA encoding SpCas9, Cas12b, CBE, and other ABEs; in all cases the DNA template, reaction conditions, and purification parameters are optimized for the specific gene of interest.

Similarly, ABE encoding mRNA with 5′ end region, 1107a, (FIG. 1B) was produced and characterized as described in the paragraph above. The final production yield for mRNA with 5′ end region, 1107a, was about 3.3 mg per 1 ml of IVT reaction (FIG. 2) and had a full-length purity of about 86% (FIG. 3).

Similarly, exemplary trinucleotide 5′ end initiators 1007e, 1007i, 1002e, 1002i, 1032a, 1037a, 1112a, 1137a, 1141a, 3102a, 3107a, 3207a, 3212a, 1007c, 1107c, 1112c, 3102c, 3107c, 3207c, 3212c, 5212, 5213, and 5214 (described in Table 1) that include additional modifications to the triphosphate bridge, phosphodiester linkages, ribose ring functional groups, and tetranucleotide bases and other modifications are used to produce 5′ capped mRNA as described above.

Similarly, exemplary tetranucleotide 5′ end initiators 5201, 5202, 5203, 5204, 5205, 5206, 5207, 5208, 5209, 5210, and 5211 (described in Table 1) are used to produce 5′ capped mRNA as described above.

Similarly, in other embodiments, trinucleotide and tetranucleotide 5′ end initiators shown in Table 1 can be used to produce 5′ capped mRNA as described in the paragraph above, and As noted by this example and the data generated, these will exhibit similar properties based on the results of this example.

B. Adjustment of mRNA 5′ End Initiator-to-NTP Molar Ratio

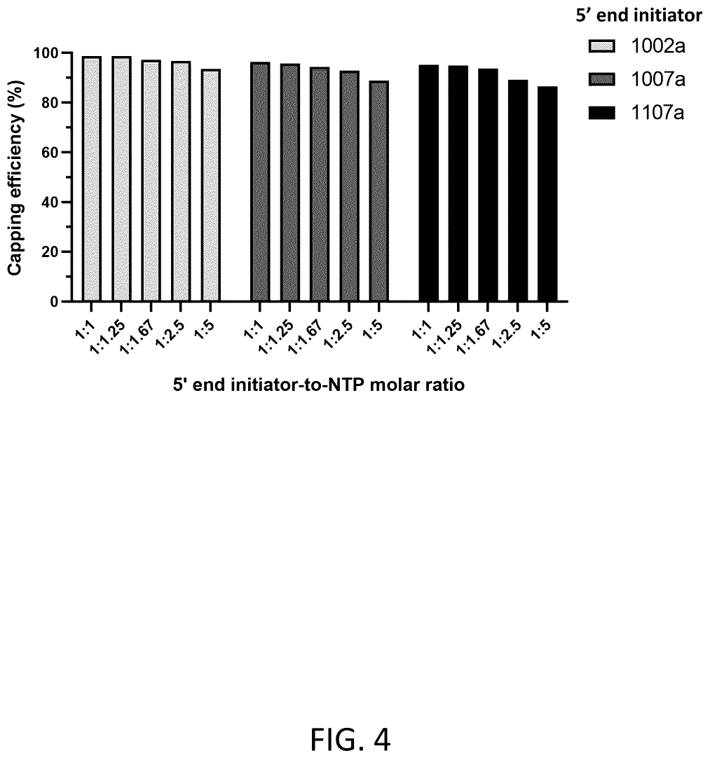

The amount of capped mRNA produced in an IVT reaction is directly proportional to the ratio of cap to NTPs in the reaction mixture (Stepinski, J. et al., RNA. 2006, 7, 1486-1495). The 5′ end initiator-to-NTP ratio in an IVT reaction impacts the amount of mRNA that becomes capped primarily because cap can compete with NTPs for insertion at the 5′ end of mRNA. A typical initiator-to-NTP ratio for a dinucleotide cap structure is 4:1 and a typical molar initiator-to-NTP ratio for a trinucleotide cap structure is 1:1; where NTP represents either GTP, ATP, CTP, UTP, or modified NTPs such as N1-methyl pseudouridine. At both a 4:1 ratio for a dinucleotide cap and 1:1 ratio for a trinucleotide cap, the resultant mRNA will have a mixture of transcripts of which at least 80% are capped at the 5′ end and up to 20% are uncapped, this is also described as a capping efficiency of 80%. Capping efficiency is the measured amount of capped mRNA divided by the total amount of capped mRNA plus uncapped mRNA. Therefore, trinucleotide caps can be considered more efficient capping compounds than dinucleotide caps since less input cap is required to produce a final mRNA product that contains a similar amount of capped 5′ ends. The ability of trinucleotide caps to produce a high (>90%) capping efficiency at a lower initiator-to-NTP ratio than dinucleotide caps, in some instances, can be attributed to the increased affinity of a trinucleotide cap for the DNA template. In some cases, the optimal cap-to-NTP ratio may vary with the composition of the cap structure, wherein modification of cap structures may affect capping efficiency. For example, cap structures with phosphorothioate backbones modifications may exhibit reduced capping efficiency at a 1:1 initiator-to-NTP ratio compared to conventional phosphodiester backbones since phosphorothioate groups can weaken hybridization strength of complementary nucleic acids. Unexpectedly, mRNA capped with phosphorothioate-containing compound 1007a had a capping efficiency of greater than 90% at an initiator-to-NTP ratio ranging from 1:1 down to 1:2.5 (FIG. 4); this was similar to the capping efficiency of mRNA capped with the conventional phosphodiester-containing 5′ end initiator, 1002a, using similar production conditions. Similarly, trinucleotide initiators, 1007e, 1007i, 1037a, 1112a, 1141a, 3107a, 3212a, 1007c, 1112c, 3107c, 3212c, 5214 and other trinucleotide cap structures described in Table 1 with phosphorothioate linkage between the first two initiating nucleotides are expected to also have similar capping efficiencies as compared to compound 1002a based on the observations seen in the previous example.

Phosphorothioate modification of the triphosphate bridge in a cap structure is not expected to reduce capping efficiency and production of mRNA containing triphosphate bridge-modified dinucleotide caps is well known in the art. Interestingly, mRNA was produced with cap compound 1107a (FIG. 2), which demonstrated that trinucleotide caps containing phosphorothioate modification of the triphosphate bridge can be incorporated into mRNA with >90% capping efficiency at an initiator-to-NTP ratio ranging from 1:1 down to 1:1.67 (FIG. 4). Similarly, trinucleotide cap structures with triphosphate bridge phosphorothioate modifications described in Table 1 are expected to have similar capping efficiencies as compared to 5′ end initiator, 1107a, based on the observations seen in the previous example.

Additionally, tetranucleotide cap compounds 5201, 5202, 5203, 5204, 5205, 5206, 5207, 5208, 5209, 5210, 5211, and other cap structures described in Table 1 may also produce mRNA with >90% capping efficiency at an initiator-to-NTP ratio of 1:1 and below due to the increased hybridization strength of the tetranucleotide cap compared to trinucleotide and dinucleotide caps.

Furthermore, as seen with mRNA made with compounds 1007a and 1107a, an initiator-to-NTP ratio ranging from 1:1 down to 1:5 did not reduce the amount of full-length mRNA produced (FIG. 3), and so the chemically modified cap compounds described herein were able to function as mRNA initiators across a, range of reaction conditions. As noted by this example and the data generated, it is contemplated that the remaining compounds described in Table 1 will exhibit similar properties based on the results of this example.

C. Determination of mRNA Capping Efficiency

The amount of capped mRNA relative to the total amount of mRNA that is present, i.e. capping efficiency, can be measured by numerous methods well known in the art. One of such methods is digestion of the mRNA into small fragments followed by chromatographic separation and detection. In some cases, digestion is performed with sequence-specific RNA or DNA nucleases or cleavage enzymes. Chromatographic separation and measurement of capped RNA is typically performed by HPLC, FPLC, thin layer chromatography, gel electrophoresis, or capillary gel electrophoresis techniques. Identity of capped RNA and uncapped mRNA is verified by mass spectrometry.

### Example 8. Measurement of Capped mRNA Stability

Removal of the cap from mRNA, i.e. decapping, is a common initiating step in the degradation of mRNA within cells. Reported methods for measuring decapping and mRNA degradation are used to evaluate the stability of capped mRNA (Grudzien, E. et al., J. Biol. Chem. 2006, 281, 1857-1867). Cap structures that prevent the interaction of decapping enzymes may improve mRNA stability and half-life in cells. For example, mRNA with 5′ end region motifs, 1032a, 1037a, 1107a, 5207a, 1112a, 1137a, 1141a, 5208, 3207a, 3212a, 1107c, 1112c, 3207c, and 3212c, contain phosphorothioate modifications in the triphosphate linkage that may inhibit the activity of dcp2 decapping enzyme. Similarly, other compounds as described in Table 1 containing phosphorothioate modifications in the triphosphate linkage may also inhibit the activity of dcp2 decapping enzyme based on the results of the previous examples. Stability of mRNA may also be improved with cap modifications that inhibit exonuclease activity. For example, introduction of phosphorothioate modifications between the first two or three initiating nucleotides in the cap structure, as present in mRNA with 5′ end region motifs, 1007a, 1007e, 1007i, 1037a, 1112a, 1141a, 3107a, 3212a, 1007c, 1112c, 3107c, 3212c, 5203, 5204, 5205, 5206, 5209, 5210, 5211, 5214, may reduce the rate of mRNA degradation in cells. Similarly, other compounds as described in Table 1 with a phosphorothioate modification between the first two or three initiating nucleotides in the cap structure may also reduce the rate of mRNA degradation in cells as seen by the previous example.

### Example 9. Translation of Capped Luciferase mRNA in HeLa Cells and Lysates

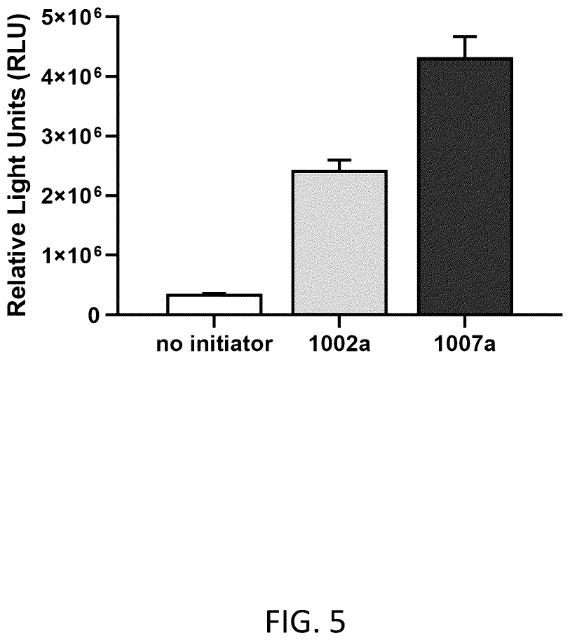

HeLa cells are cultured in RPMI with 10% FBS at 37° C. under 5% CO₂ atmosphere. Cells are plated into an appropriately sized well plate per manufacturer's recommended seeding conditions. After overnight incubation, cells are checked for monolayer formation and then transfected using a transfection agent such as MessengerMAX. Solution A: the desired amount of luciferase mRNA is diluted in OptiMEM and mixed with Solution B: MessengerMAX in OptiMEM. After mixing solutions A and B, the mixture is incubated at room temperature for 20 minutes, and then added dropwise to each well of cells. The cells are then incubated at 37° C. for 12-72 hours to allow for expression of mRNA into protein. Cells are harvested and prepared for measuring luciferase protein levels per Promega luciferase assay system protocol. In some instances, mRNA is directly administered to HeLa lysates that contain the cellular components for protein expression, this approach does not require the use of a transfection agent. For example, luciferase encoding mRNA with 5′ end region motif, 1007a, was added to a commercially available HeLa lysate in vitro translation kit to produce luciferase protein. Luciferase protein activity as measured by luminescent light production in the presence of a luciferin substrate, was measured with a luminescence-detecting plate reader. Relative light units measured per this assay are proportional to the amount of luciferase protein translated from mRNA. The results of this Example were compared using a mRNA made with a commercially available 5′ end initiator, 1002a, as a reference. In this example, the mRNA with initiator 1007a produced around 2× greater luciferase activity than the mRNA with initiator 1002a (FIG. 5). As noted by this example and the data generated, it is contemplated that the remaining compounds described in Table 1 will exhibit similar properties based on the results of this example.

### Example 10. Translation of Capped mRNA in Primary Hepatocyte Cells for Genome Editing

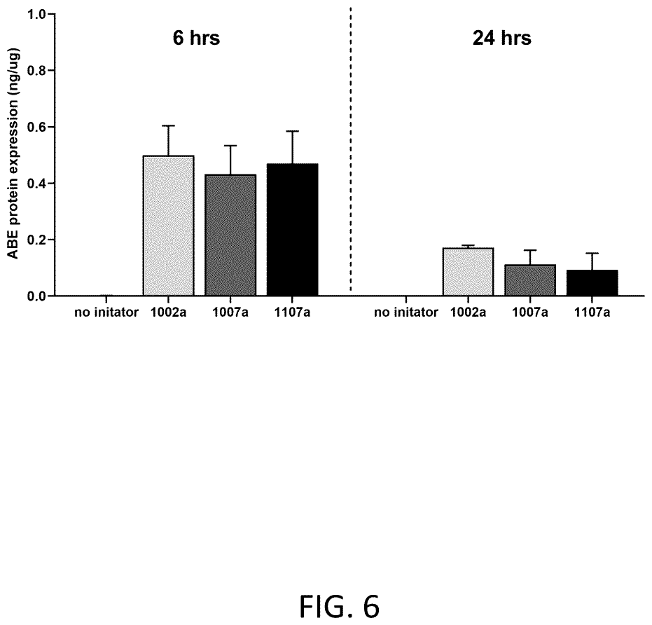

Primary human liver hepatocytes (hPH), primary cynomolgus liver hepatocytes (cPH), or primary mouse liver hepatocytes (mPH) are cultured per the manufacturer's protocol. In brief, the cells are thawed and resuspended in hepatocyte thawing medium followed by centrifugation at 100 g for 10 min at 4° C. The supernatant is discarded, and the pelleted cells resuspended in hepatocyte plating medium. Each vial contains ˜5 million cells that are used for plating one 24-well plate. Plated cells are allowed to settle and adhere for 4-6 h in a tissue culture incubator at 37° C. under 5% CO₂ atmosphere. After incubation, cells are checked for monolayer formation. The incubating media is then replaced with fresh hepatocyte maintenance media The cells thus are ready for transfection using a transfection agent such as MessengerMAX. Solution A: desired amount of single guide RNA (sgRNA) is mixed with 1:1 wt ratio of mRNA in OptiMEM. Solution B: MessengerMAX in OptiMEM. After mixing solutions A and B, the mixture is incubated at room temperature for 20 minutes, and then the incubated solution is added dropwise to each well of cells. The cells are then allowed to remain at 37° C. for 3 days. Cells are harvested and prepared for genomic DNA extraction using Thermo Kingfisher instrument. Extracted genomic DNA is processed by PCR, and amplified PCR product was then subjected to NGS (MiSeq) to analyze genome editing. In some instances, hPH are transfected with ABE encoding mRNA as described above and the levels of translated ABE protein are used to assess the mRNA function. For example, mRNA with 5′ end region motifs, 1007a and 1107a, were transfected to hPH cells as described above, the cells were lysed, and ABE protein levels were measured by ELISA. The results of this example were compared using ABE mRNA made with a commercially available initiator, 1002a, as a reference. In this example, the mRNAs with 5′ end region motifs, 1007a and 1107a, produced similar ABE protein levels as compared to the mRNA with 5′ end region motif, 1002a (FIG. 6). As noted by this example and the data generated, it is contemplated that the remaining compounds described in Table 1 will exhibit similar properties based on the results of this example.

### Example 11. Translation of Capped mRNA In Vivo in Mouse and/or Non-Human Primates (NHP) for Genome Editing

### A. LNP Preparation for In Vitro Transfection and In Vivo Evaluation

Lipid nanoparticles (LNPs) are generated by microfluidic mixing using the Precision Nanosystems NanoAssemblr system according to the manufacturer's protocol, with some optimization for individual payloads. The resulting LNPs are filtered using 0.2-micron filters and stored at 4° C. or frozen at −80° C.

### B. In Vivo Evaluation in Mice

LNPs used in these studies were prepared as described above in Example 11 A, comprising chemically synthesized sgRNAs targeting the mouse ANGPTL3 (mANGPTL3) and/or mouse PCSK9 (mPCSK9) genes and SpCas9 or ABE8.8 mRNA in a 1:1 weight ratio. LNPs were administered to C57BL/6 mice (n=5) intravenously via the lateral tail vein or retro-orbital sinus in a total volume of 5-10 ml/kg, or about 0.05 mg/kg. 4, 5, or 7 days post dose at necropsy, livers and blood were collected for NGS measurements of editing efficiency, and serum PCSK9, triglyceride and cholesterol analyses.

### C. In Vivo Evaluation in NHP

LNPs used in these studies were prepared with sgRNA targeting ANGPTL3 and ABE encoding mRNAs with 5′ end region motifs as described in Example 11A, comprising chemically synthesized sgRNAs targeting the NHP ANGPTL3 genes and ABE8.8 mRNA in a 1:1 weight ratio. LNPs were administered to NHP (n=3 per group) by one single intravenous infusion in a total volume of 6 mL/kg. Livers were collected 16 days post dose for NGS measurements of editing efficiency.

### D. Genomic DNA Isolation

Mouse whole liver or NHP whole livers are loaded into 2 mL lysing matrix tubes with 0.5 mL of PBS. The livers are homogenized using the FastPrep-24 system according the to manufacturer's protocol. Genomic DNA is isolated from approximately 20 microliters of whole mouse liver lysate or NHP liver homogenate using a bead-based extraction kit, MagMAX-96 DNA Multi-Sample Kit on the KingFisher Flex automated extraction instrument according to the manufacturer's protocols. Extracted genomic DNA is stored at 4° C. until further use or at −80° C. for long term storage.

### E. Next Generation Sequencing (NGS) and Analysis of Editing Efficiency

Next generation sequencing, or deep sequencing, was performed on the region of interest to determine the extent of gene editing. Samples are prepared using the Nextera XT DNA library preparation kit (Illumina) according to the manufacturer's protocol. Briefly, two rounds of PCR are performed first to amplify the region of interest and second to add DNA sequences required for deep sequencing and sample identification to the initial product. The final amplicon is sequenced on the Illumina MiSeq instrument according to the manufacturer's protocol.

Paired-end reads are analyzed with the CRISPResso2 pipeline (Clement, K. et al., Nat. Biotech. 2019, 37, 224-226). Briefly, low-quality reads are filtered out, adapter sequences are trimmed from the reads, and the paired-end reads are merged and aligned to the amplicon sequence. The editing percentage is calculated as the number of reads supporting an insertion or a deletion, over the total number of aligned reads. For ABE, the editing percentage is calculated as the number of reads supporting an insertion or a deletion, over the total number of aligned reads.

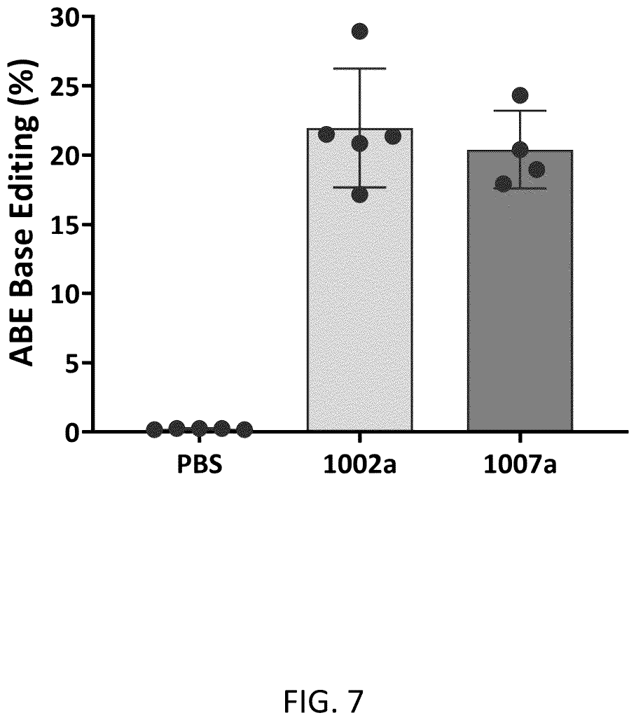

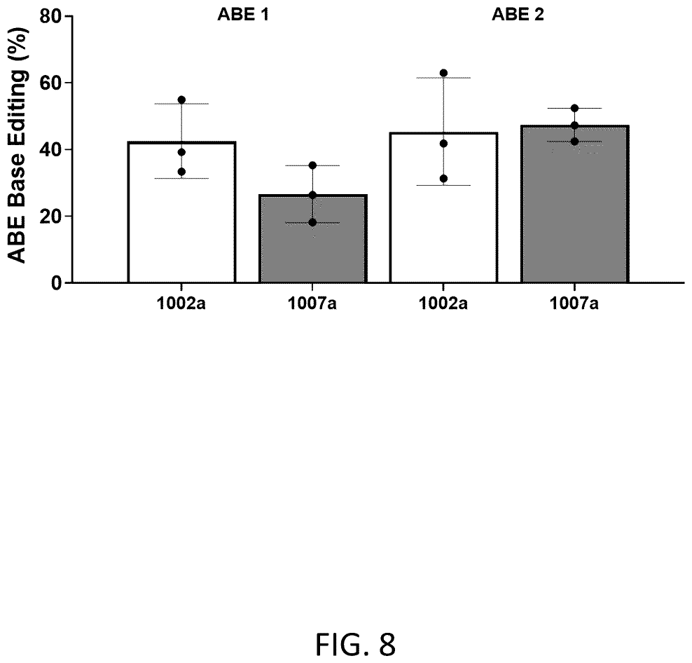

In some instances, LNPs were formulated with sgRNA targeting mPCSK9 and ABE encoding mRNAs with 5′ end region motif, 1007a, administered in vivo to mice, and editing efficiency was assessed as described in Example 11. The results of this example were compared using ABE encoding mRNA made with a commercially available 5′ end initiator, 1002a, as a reference. In this example, the mRNA with 5′ end region motif, 1007a, produced similar hepatocyte editing levels as compared to the mRNA with 5′ end region motif, 1002a (FIG. 7). FIG. 20 further illustrates the results of this example using mRNA with 5′ end region motif 5231, 5232, 1007a, 1007a_D1, and 1007a_D2 in reference to commercially available 5′ end initiator 1002a. All mRNA exhibited similar levels of editing efficiency in this illustration. In some instances, LNPs were formulated with sgRNA targeting NHP ANGPTL3 and ABE encoding mRNAs with 5′ end region motifs, 1007a and 1002a, administered in vivo to NHP, and editing efficiency was assessed as described in Example 11. ABE mRNAs with 1007a 5′ end region motif showed lower or similar base editing efficacy to mRNAs with 1002a 5′ end region motif (FIG. 8).

As noted by this example and the data generated, it is contemplated that the remaining compounds in Table 1 will exhibit similar properties based on the results of this example.

### Example 12. Determination of Immune Stimulation of Capped mRNA In Vivo in Mouse or in an In Vitro HEK293 Cell Model

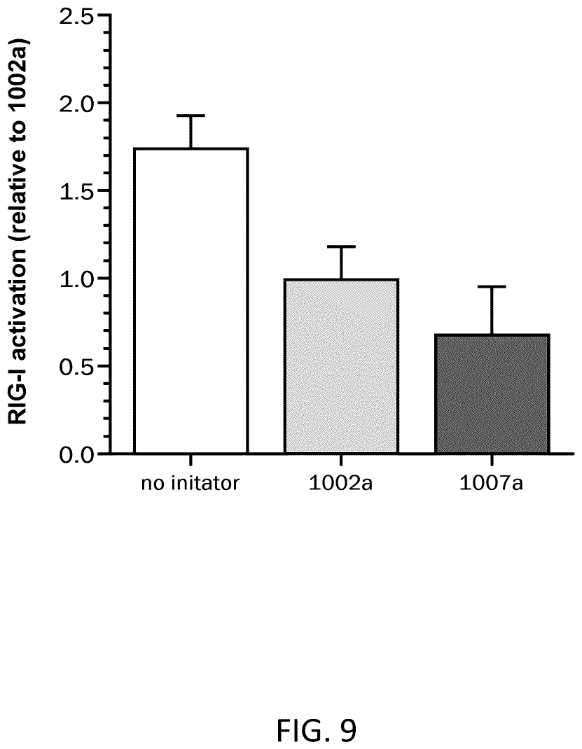

LNPs are prepared and administered in vivo to mice as in Example 11(A,B). Blood is collected at appropriate time points, typically at intervals between 1-48 hours post-dose and terminal end point, and processed to serum or plasma per techniques well known in the art. Serum or plasma cytokine and chemokine levels are measured using V-PLEX cytokine and chemokine detection kits. Cytokine and chemokine levels are compared to animals treated with buffer alone as well as pre-dose sampling of the treatment animals. In some instances, the immune stimulation of capped mRNA is assessed after in vitro transfection to reporter cell lines such as HEK-Lucia RIG-I cell reporter model, which produces luciferase signal in the presence of IFN-stimulated response elements such as uncapped mRNA. This cell model is cultured and transfected as described in Example 7, and luciferase activity is measured using a luciferase activity kit and plate reader. It is known in the art that uncapped mRNA produces a RIG-I-dependent IFN immune response, and capped mRNA lessens this immune stimulation. In this example, mRNA with 5′ end region motif, 1007a, showed reduced RIG-I activity as compared to uncapped mRNA using the HEK-Lucia RIG-I cell reporter model (FIG. 9). Furthermore, mRNA with 5′ end region motif, 1007a, did not appear to increase RIG-I activation as a result of the chemical modifications to the cap structure. The results of this example were compared using mRNA made with a commercially available 5′ end initiator, 1002a, as a reference. As noted by this example and the data generated, it is contemplated that the remaining compounds in Table 1 will exhibit similar properties based on the results of this example.

### Example 13. Preparation of Trinucleotide Caps 5231, 5232, 5233, 5234, 5235, and 5236

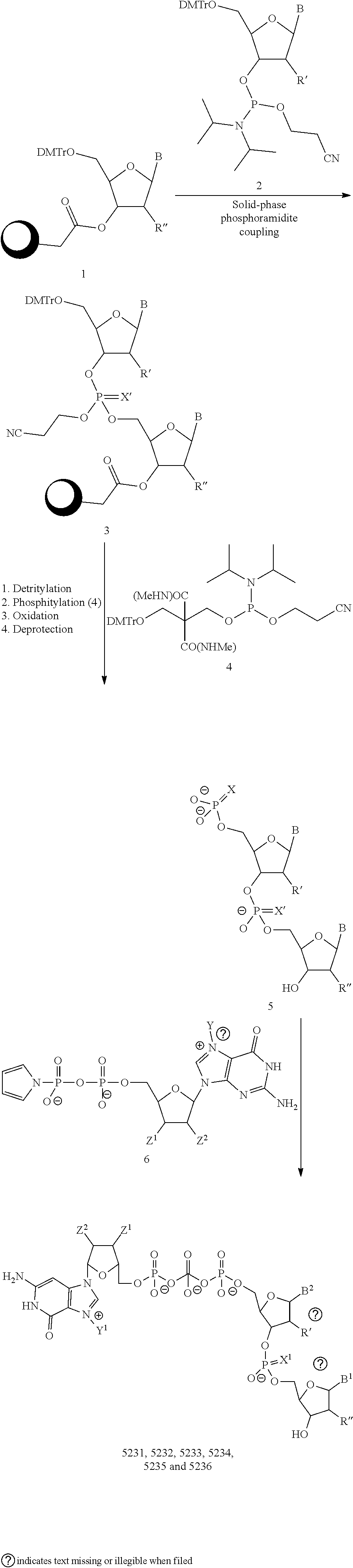

Where B¹=guanine; B²=2,6-diaminopurine or adenosine; X=O or S; and Y¹═CH₃ or 4-chlorobenzyl (4-Cl-Bn).

The dinucleotide 5′-phosphate 5 is prepared from the solid support 1 and commercially available adenosine or 2-aminoadenosine 3′-phosphoramidite 2 by following standard solid phase oligonucleotide synthesis and deprotection protocol (Methods in Molecular Biology, 1993, 20, 81-114; ACS Chem. Biol. 2015, 10, 1181-1187, incorporated herein by reference in its entirety). The detritylation of support bound fully protected dinucleotide 3 affords the 5′-deprotected dinucleotide intermediate on the support. The intermediate thus obtained is reacted with the 5′-phosphorylating agent 4 according to reported procedures (EP 816368 A1, https://www.glenresearch.com/solid-chemical-phosphorylation-reagent-ii.html, The Glen Report, 2011, 23, 10-11). After coupling and oxidation and detritylation, the 5′-phosphorylated dinucleotide is treated with base followed by HPLC purification to obtain the fully deprotected dinucleotide 5′-phosphate 5 (https://www.glenresearch.com/10-1902.html). Compound 6 is prepared from imidazole-activated m⁷GDP or 7-(4-chlorobenzyl)-GDP 6. 7-(4-chlorobenzyl)-GDP 6 is prepared from guanosine as reported (Pharmaceutics, 2021, 13, 1941; DOI: 10.3390/pharmaceutics13111941).

Compound 6 is coupled with the dinucleotide 5′-monophosphate 5 as described in the literature (Nucleic Acids Research, 2016, 44, 9578, WO 2017/053297 A1) to afford the desired compounds 5231, 5232 and 5233. Substitution of phosphite to phosphate oxidizer with a sulfur transfer reagent, 3-[(dimethylaminomethylene)amino]-3H-1,2,4-dithiazole-5-thione (DDTT) for example, during the preparation of solid-bound dinucleotide 3 affords compounds 5234, 5235, and 5236.

Compounds 5231, 5232, 5233, 5234, 5235, and 5236 may be employed as 5′ end mRNA initiators as described in Example 7. For example, compound 5231 can be used to increase mRNA potency, in that an interaction between compound 5231 and critical ribosome initiation factor elF4E can occur through stabilized contact with the protein binding pocket and the N7 position of the modified guanine. Increased binding potential may increase mRNA potency by lowering the rate-limiting initiation barrier of the protein translation process. For example, compound 5232 can be used to improve efficiency at which mRNA possesses the 5′ end initiating analog by increasing the hybridization potential over that of competing nucleotides in the RNA synthesis reaction.

One or more of the described compounds, 5231, 5232, 5233, 5234, 5235, and 5236, may show capping efficiencies such as those described in Example 7.

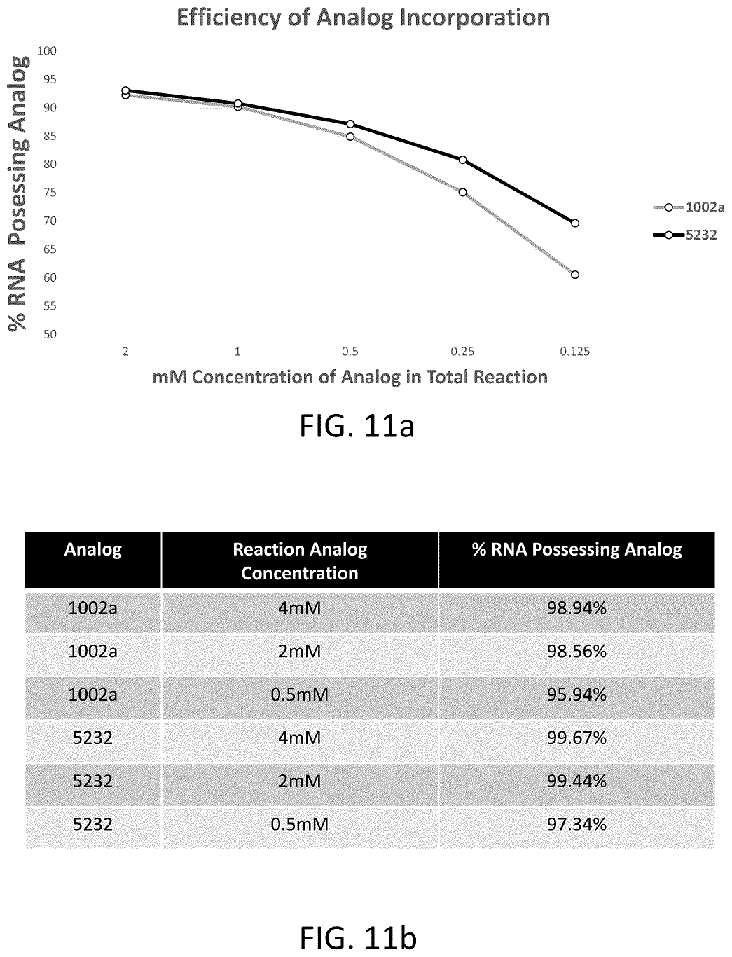

Trinucleotide cap compound 5232 may also produce mRNA with >90% capping efficiency at an initiator-to-NTP ratio of 1:1 and below. For example, in FIG. 11a, compound 5232 caps a greater percentage of RNA at a concentration of 0.125 mM or greater, compared to 1002a. Particularly in FIG. 11b, compound 5232 caps at least 97% of RNA at a concentration of 0.5 mM, whereas 1002a caps about 96%. The additional amine group on the second nucleotide base may cooperate in hydrogen bonding with the DNA template during RNA synthesis reaction.

### Example 14: mRNA Yield and Quality

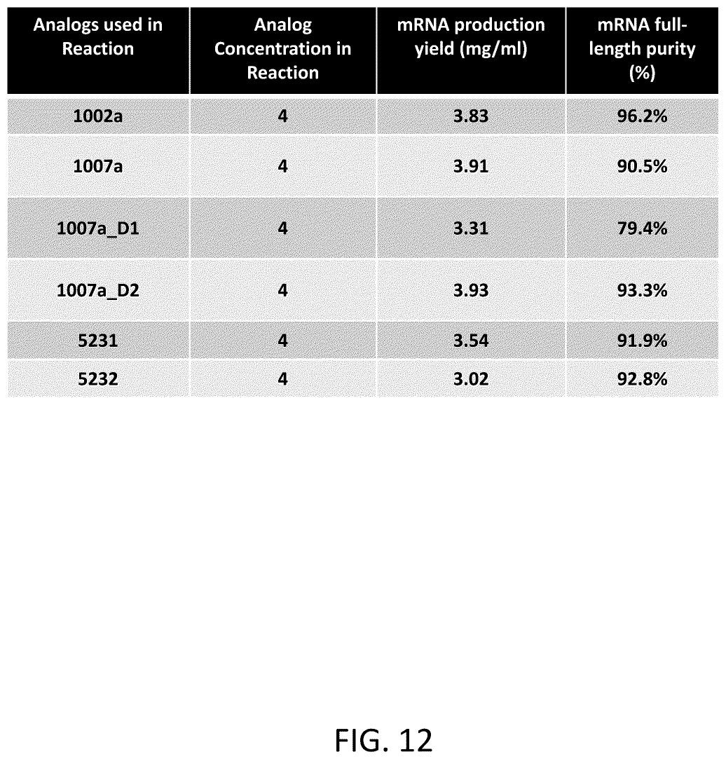

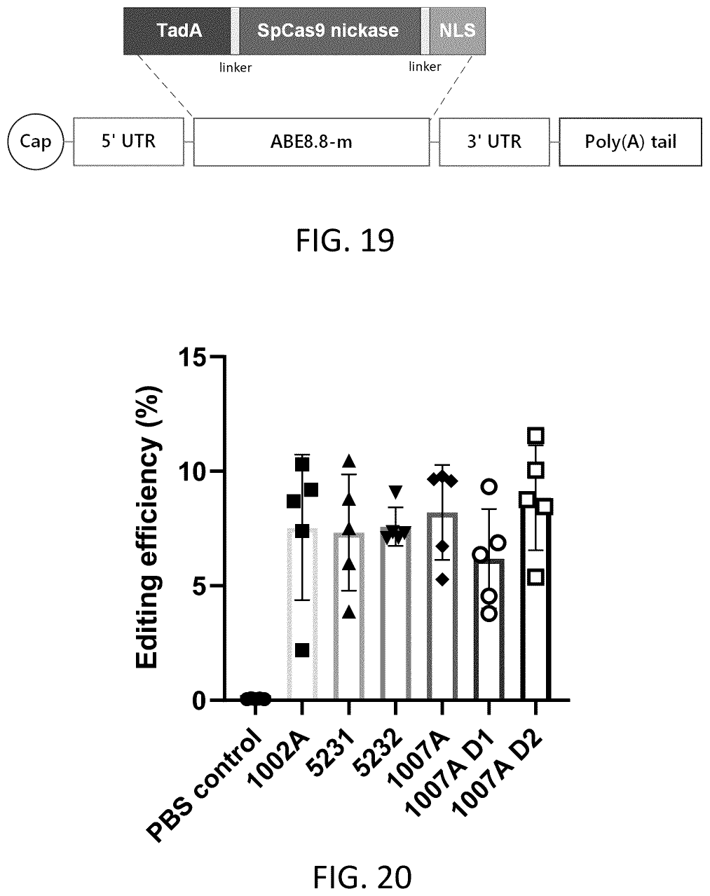

RNA synthesis reactions were established with constate substrate and enzyme concentrations as shown in FIG. 12. In this Example and for all the rest described herein, the ABE mRNA sequence was MA079 (SEQ ID NO: 4). The reactions were established using 4 mM of each test analog (1007a, 1007a_D1, 1007_D2, 5231, and 5232) and synthesis was allowed to proceed for 90 minutes. mRNA production yield (mg/ml) was determined by measuring the UV absorption of a known volume of sample compounded by the original reaction volume. The mRNA full-length purity (FLP) % was determined by electrophoretic separation of mRNA and a known sample. Upon separation, the mobility of mRNA species within the product was compared to that of the known standard to determine the size of the polymer. The absorption intensity of the size-separated product was then used to quantify the population's distribution as a function of size. The expected size population was then compared to the entire population to derive a % full-length purity. As shown in the table, three cap analogs (1002a, 1007a and 1007a_D2) generated mRNA at similar concentration of 4 mg/m. mRNA yield with 5231 and 1007a_D1 cap analogs were slightly lower, while 5232 cap analog had the lowest mRNA yield at around 3 mg/ml. mRNA produced using 1002a cap has the highest FLP of 96.2%. The rest of the cap analogs produced mRNA at similar FLP at around 90% expect 1007a_D1, which was 79.4%. The structures of MA004 and MA079 are illustrated in FIG. 19, where ABE8.8-m=adenine base editor, version 8.8-m; Cap=methylated 5′-Cap-1; NLS=nuclear localization signal; Poly(A)=polyadenylic acid; SpCas9=Streptococcus pyogenes CRISPR-associated protein 9; and TadA=adenosine.

### Example 15: Immunogenicity

a. Detection of % dsRNA

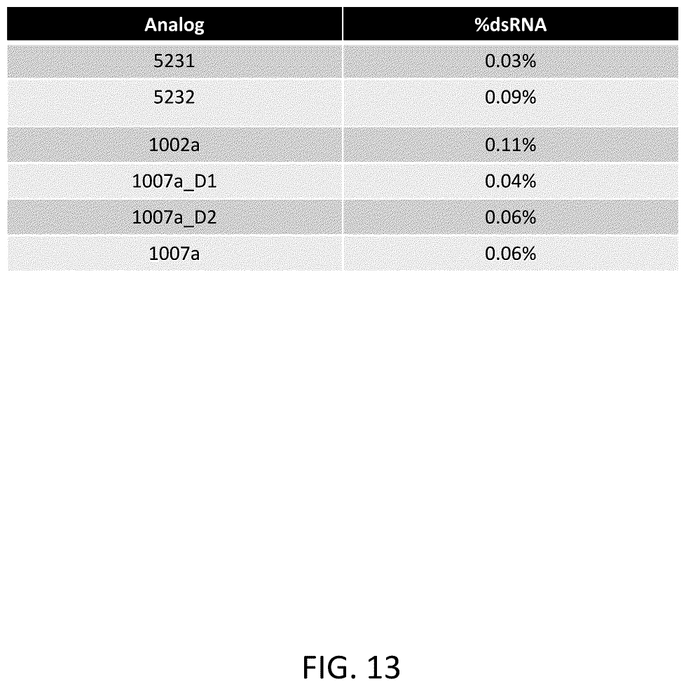

RNA-derived immunogenicity arises through the unintended production of dsRNA during the synthesis reaction, which is detected by intracellular and extracellular viral defense mechanisms. Activation of the dsRNA stress response can lead to a reduction in protein production and/or increased turnover of the RNA within the targeted cell. Immunogenicity can affect potency in addition to being a serious concern for safety. Mitigation of dsRNA generation during the RNA synthesis process can be realized through the manipulation of substrate concentrations, such as 5′ initiator concentration. As shown in FIG. 13, test analogs (1007a, 1007a_D1, 1007_D2, 5231, and 5232) were studied in regards to generated dsRNA during synthesis. The % dsRNA reflects the population of mRNA that is found to be dsRNA in a known sample quantity. These values were determined via a sandwich ELISA and known quantity dsRNA standards. % dsRNA were the highest with 5232 and 1002a among all the tested cap analogs. 5231 cap analog indicated the lowest dsRNA content (0.03%), which was about 0.01% or 0.03% lower than 1007a_D1 and 1007a analogs.

b. RIG-I Response

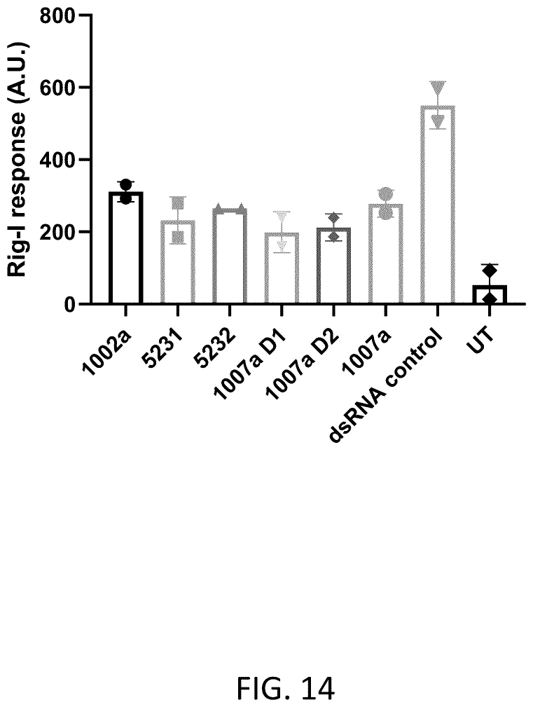

As shown in FIG. 14, the results of Retinoic acid-inducible gene I (RIG-I) response for test analogs (5231, 5232, 1007a_D1, 1007a_D2, 1007a). RIG-I overexpressed reporter HEK293 cells were utilized to evaluate cap specific immune response. mRNA with different cap analogs were transfected to the reporter cells. This cell model is cultured and transfected as described in Example 7. The response was measured at 24 hours after transfection. RIG-I is a cytosolic pattern recognition receptor that is responsible for the type-1 interferon response and can respond to 5′ triphosphate of RNA. Of the test analogs, 1002a had the highest RIG-I response and 1007a_D1 had the lowest RIG-I response The rest of the cap analogs shared similar RIG-I signals.

c. Cytokine Measurements

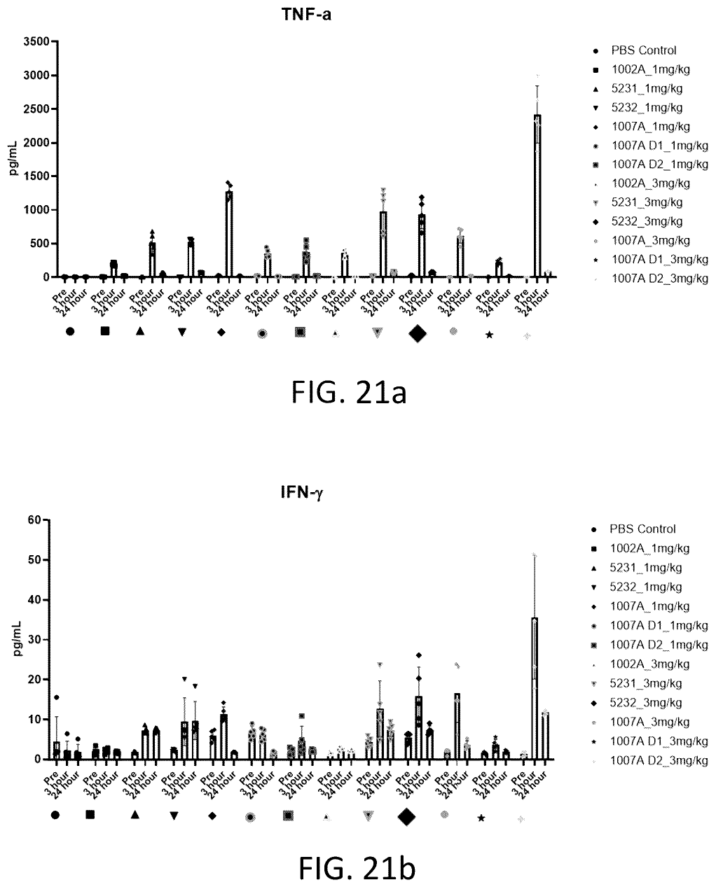

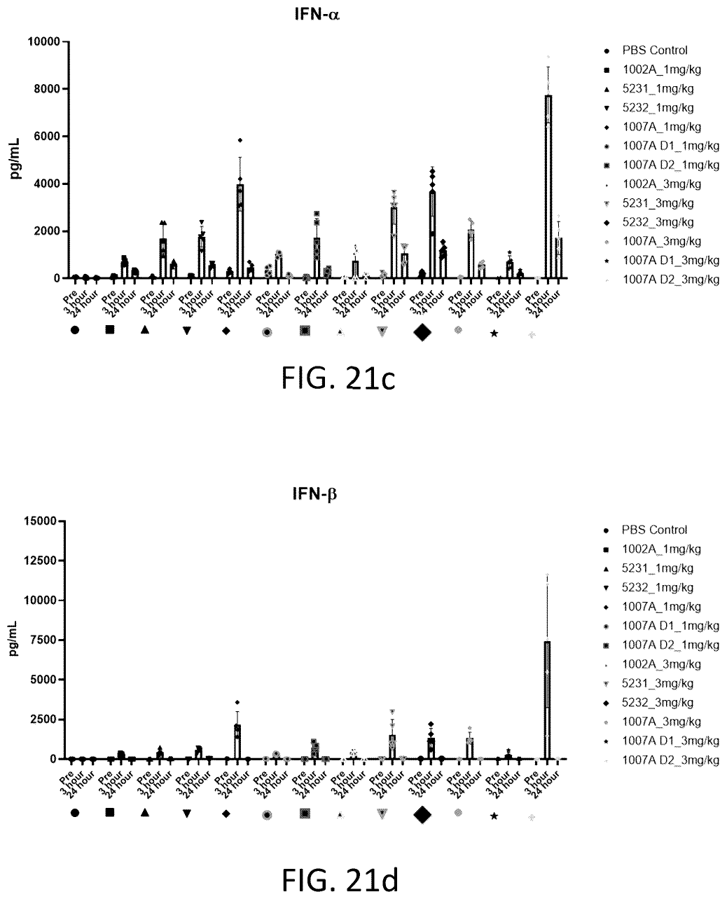

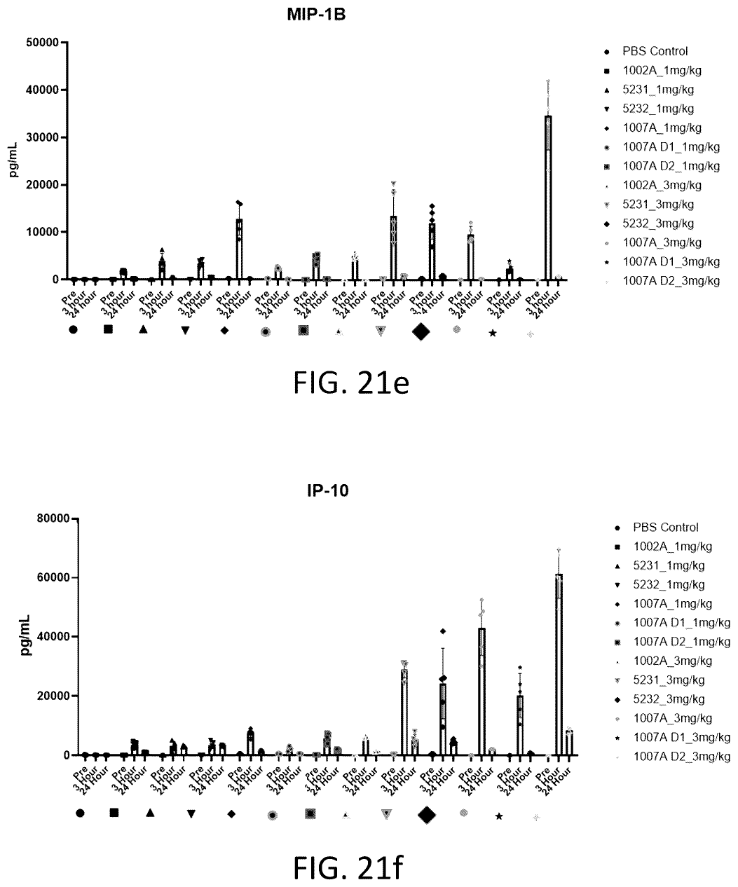

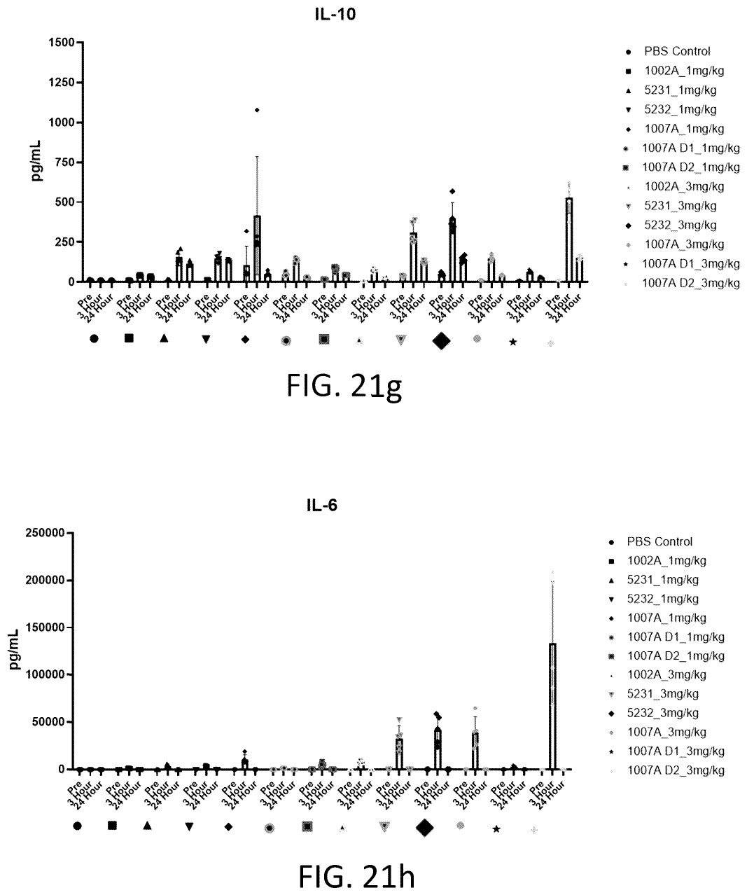

In vivo immunogenicity was quantified as described in Example 12. Mice were treated with LNPs at a level of 1 mg/kg and 3 mg/kg, and serum was collected 3 hours and 24 hours post-injection. As shown in FIGS. 21a-21h, all cytokines measured (TNF-α, IFN-γ, IFN-α, IFN-β, MIP-1B, IP-10, IL-10, and IL-6) were detectable at 3 hours post-injection and returned to baseline (i.e., about pre-injection level) at 24 hours post-injection for mRNA comprising 5′ end region motifs 5231, 5232, 1007a, 1007a_D1, and 1007a_D2. For comparison, cytokine measurements were also completed for mRNA comprising commercially available 5′ end initiator 1002a. In some cases, the mRNA comprising 5′ end region motifs 5231, 5232, 1007a, 1007a_D1, and 1007a_D2 exhibit a similar immunogenicity profile.

### Example 16: Potency

a. Nuclease Resistance

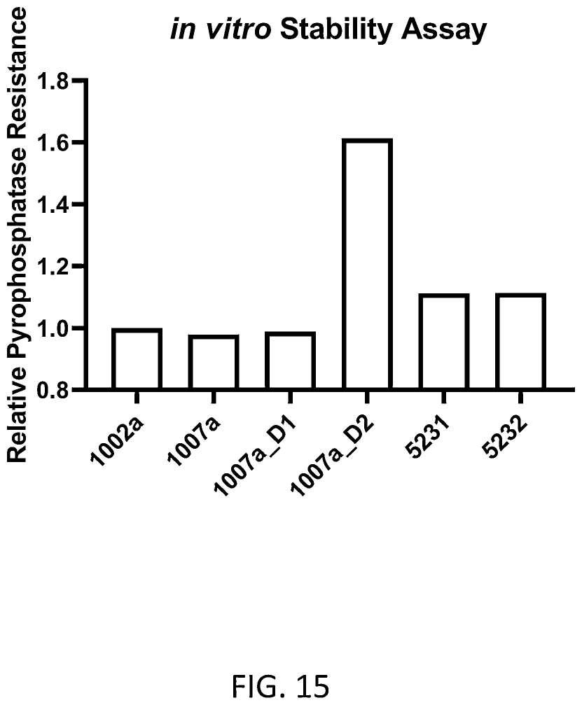

In vitro stability was determined for 5′ initiator analogs (1007a, 1007a_D1, 1007a_D2, 5231, and 5232) along with 1002a with regards to relative pyrophosphatase resistance, as shown in FIG. 15. The performance of a 5′ initiator analog intended for the cellular expression of proteins upon delivery into the cell may depend, at least in part, on that analog's ability to resist cleavage by enzymes (e.g., pyrophosphatases) that recognize the 5′-5′ triphosphate bridge. Pyrophosphatases may recognize the unique electronegative architecture of the triphosphate along with one or more other contacts on the bound RNA molecule. To test the susceptibility of these analogs to pyrophosphatases, the in vitro experiment utilizes the commercially available recombinant yeast pyrophosphatase, DcpS, incubated with known concentrations of analog: 8 μM, 16 μM, 24 μM, 32 μM, and 40 μM. Cleavage of the triphosphate bridge exposes phosphates that may be liberated into a solution with commercially available alkaline phosphatase. The released phosphate may be detected using a malachite green reagent, which can adopt a colorimetric shift upon complexation with a free phosphate. The extent of pyrophosphatase cleavage may be gauged by the extent of malachite green activation. The absorption values of the tested analogs (1007a, 1007a_D1, 1007a_D2, 5231, and 5232) were relatively compared to that of 1002a to interpret the extent to which the structural differences changed the resistance profile. The inverse absorption values were taken to reflect the % change in pyrophosphatase resistance. As shown in FIG. 15, 1007a_D2 appears to be the most pyrophosphatase resistant one among all the tested cap analogs. 5231 and 5232 had similar performance, which were more stable than 1002a, 1007a and 1007a_D1 analogs.

b. Ribosome Recruitment

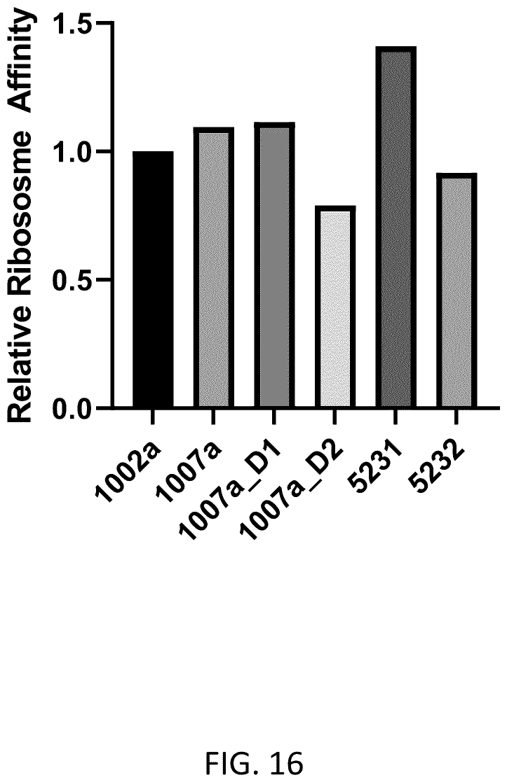

Relative ribosome affinity was assessed for initiator analogs (1007a, 1007a_D1, 1007a_D2, 5231, and 5232) along with 1002a, as shown in FIG. 16. Rabbit reticulocyte lysate is prepared, which is a crude cell extract that may contain all the components necessary for protein synthesis, including ribosomes and various initiation factors like eIF4E. The lysate is then incubated with a known quantity of a cas9-bearing transcript bearing the test analogs and a known quantity of luciferase encoding mRNA. Increasing amounts of transcript bearing the test analog increasingly compete for available ribosome factors in the lysate. This competition is quantified by measuring the relative fluorescent units derived at the analog-bearing transcript. The % inhibition of signal is determined by comparing the analog based sample to a control possessing only luciferase mRNA. The y-axis represents the extent to which the signal was inhibited relative to 1002a and can be used as a surrogate for ribosome affinity. Testing ribosome recruitment may be an important factor to determine if an analog can effectively mimic the function of the natural mRNA cap in recruiting ribosomes. The mRNA cap may play a key role in regulating the efficiency and accuracy of protein synthesis. The potential effectiveness of a novel cap analog as a therapeutic agent or as a tool for manipulating protein synthesis in cells may be determined through assessing ribosome recruitment. As compared to other tested cap analogs, 5231 analog indicated the most efficient ribosome recruitment. 1002a, 1007a and 1007a_D1 analogs were slightly less efficient, while 1007a_D2 indicated the lowest ribosome affinity (FIG. 16).

c. Temporal and Dose-Dependent Expression of Luciferase from RNA Synthesized with 5′ Initiator Analogs

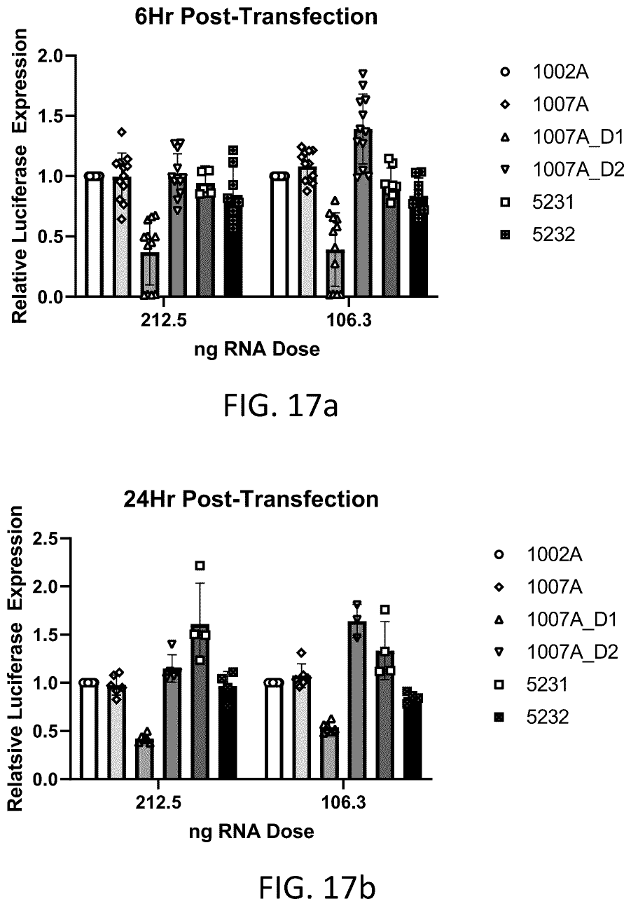

FIG. 17a shows relative luciferase expression for 5′ initiator analogs (1007a, 1007a_D1, 1007a_D2, 5231, and 5232), where cells where harvested 6 hours following exposure to mRNA. FIG. 17b shows relative luciferase expression for 5′ initiator analogs (1007a, 1007a_D1, 1007a_D2, 5231, and 5232), where cells where harvested 24 hours following exposure to mRNA. The harvested cell lysate was subjected to conditions to derive a quantifiable fluorescent signal. This signal was then compared to analog 1002a to determine how structural differences may be related to the changes in expression. Primary human hepatocytes were subjected to two different doses of mRNA encoding for the protein luciferase made using the indicated test analogs. A single RNA molecule may be interpreted by protein synthesis machinery multiple times, so an analog that may increase the frequency of interpretation may be desirable as a 5′ initiator. This frequency of interpretation, or translation efficiency, may be determined by measuring the amount of protein produced in a population of cells exposed to a known amount of RNA at a given point following that exposure. The duration of expression may also be assessed to determine how an analog may sustain the observed expression, as the RNA molecule is eventually decayed within the cell thereby destroying its protein-encoding capacity. As shown in FIG. 17a, at 6 hours after transfection, mRNA harboring most cap analogs except 1007a_D1 showed similar expression at both transfection doses. 1007a_D2 showed a little increased protein expression at 106.3 ng of mRNA dose. Similar protein expression profiles was also observed at 24 hours post transfection (FIG. 17b).

d. Temporal and Dose-Dependent Expression of Cas9 RNA Synthesized with 5′ Initiator Analog

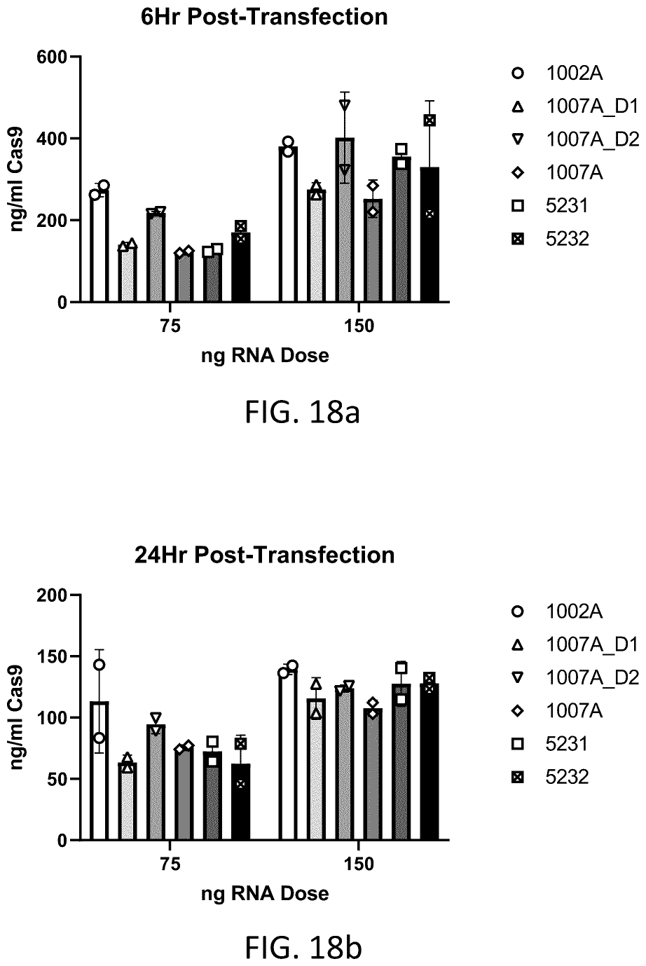

FIG. 18a shows Cas9 protein expression as a factor of RNA dose, where the mRNA is made using 5′ initiator analogs (1007a, 1007a_d1, 1007a_d2, 5231, and 5232), and where the cells are harvest 6 hours after exposure to RNA. FIG. 18b shows Cas9 protein expression as a factor of RNA dose, where the RNA is made using 5′ initiator analogs (1007a, 1007a_d1, 1007a_d2, 5231, and 5232), and where the cells are harvest 24 hours after exposure to RNA. Protein expression is directly assessed via antibody detection of the Cas9 protein. The measured signal is compared to that of a recombinant Cas9 of known concentration to derive the Y-axis value. Cas9 expression was largely similar among most analogs except 1007a_D1 and 1007a. This is consistent at both low and high doses at 6 hours post transfection. At 24 hours after transfection, the potency difference between the analogs was less pronounced, but the trend generally remained consistent.

In some cases, the mRNA comprising 5′ end region motifs 5231, 5232, 1007a, 1007a_D1, and 1007a_D2 exhibit similar potency.

### Other Embodiments

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

While specific examples and numerous embodiments have been provided to illustrate aspects and combinations of aspects of the foregoing, it should be appreciated and understood that any aspect, or combination thereof, of an exemplary or disclosed embodiment may be excluded therefrom to constitute another embodiment without limitation and that it is contemplated that any such embodiment can constitute a separate and independent claim. Similarly, it should be appreciated and understood that any aspect or combination of aspects of one or more embodiments may also be included or combined with any aspect or combination of aspects of one or more embodiments and that it is contemplated herein that all such combinations thereof fall within the scope of this disclosure and can be presented as separate and independent claims without limitation. Accordingly, it should be appreciated that any feature presented in one claim may be included in another claim; any feature presented in one claim may be removed from the claim to constitute a claim without that feature; and any feature presented in one claim may be combined with any feature in another claim, each of which is contemplated herein. The following enumerated clauses are further illustrative examples of aspects and combination of aspects of the foregoing embodiments and examples:

1. An in vitro-transcribed (IVT) mRNA sequence initiator comprising a compound of Formula (I) or a salt or solvate thereof:

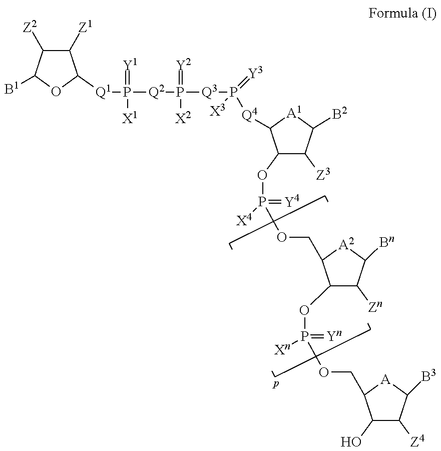

wherein
B¹ is

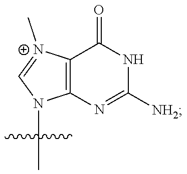 [SMILES: *n1c[n+](C)c2c(=O)[nH]c(N)nc21]

each B², B³, and Bⁿ is independently a natural, a modified, or an unnatural nucleobase;
each Z¹ and Z² is independently hydrogen, fluorine, —OH, —SH, —CH₃, —CH₂CH₃, —OCH₃, —OCH₂CH₃, —SCH₃, —NH₂, NHCH₃, or NHC(═O)CH₃;
each Z³, Z⁴, and Zⁿ is independently hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6,
provided that the compound of Formula (I) satisfies one or more of the following proviso (i) to (iii): (i) at least one of X¹, X², X³, X⁴, and Xⁿ is —SH or —S—; (ii) at least one of Y¹, Y², Y³, Y⁴, and Yⁿ is ═S; and (iii) at least one of A, A¹, and A² is —S—.

2. The IVT mRNA sequence initiator of clause 1, wherein at least one of X¹, X², X³, X⁴, and Xⁿ is —SH or —S—; at least one of Y¹, Y², Y³, Y⁴, and Yⁿ is ═S; or at least one of A, A¹, and A² is —S—.

3. An in vitro-transcribed (IVT) mRNA sequence initiator comprising a compound of Formula (II) or a salt or solvate thereof:

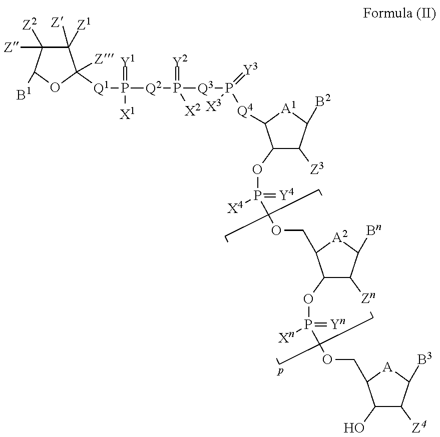

wherein
B¹ is

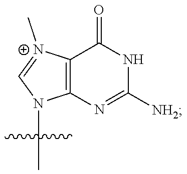 [SMILES: *n1c[n+](C)c2c(=O)[nH]c(N)nc21]

each B², B³, and Bⁿ is independently a natural, a modified, or an unnatural nucleobase;
each Z¹ and Z′ is independently is hydrogen, fluorine, —OH, —SH, —CH₃, —CH₂CH₃, —OCH₃, —NH(CH₃), —NH₂, —NH(C(═O)CH₃), or —SCH₃;
each Z² and Zⁿ is independently fluorine, —OH, —SH, —CH₃, —CH₂CH₃, —OCH₃, —SCH₃, —OCH₂CH₃, —NH₂, NHCH₃, or NHC(═O)CH₃;
Z′″ is hydrogen, fluorine, —CH₃, —CH₂CH₃, —OCH₃, or —OCH₂CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

4. An in vitro-transcribed (IVT) mRNA sequence initiator comprising a compound of Formula (II) or a salt or solvate thereof:

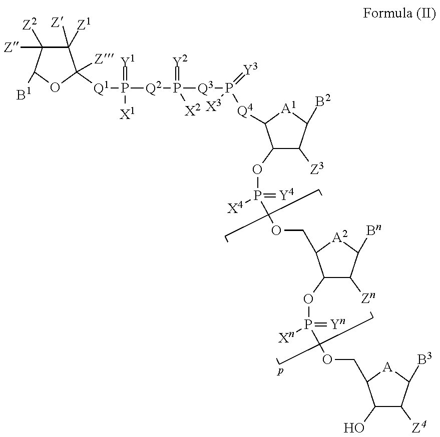

wherein
B¹ is

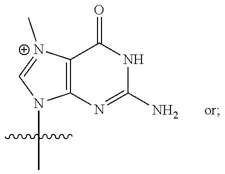 [SMILES: *n1c[n+](C)c2c(=O)[nH]c(N)nc21]

each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

5. An in vitro-transcribed (IVT) mRNA sequence initiator comprising a compound of Formula (II) or a salt or solvate thereof.

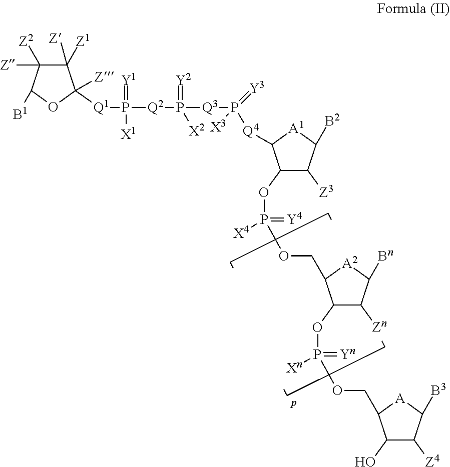

wherein
B¹ is

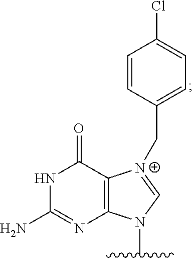 [SMILES: *n1c[n+](Cc2ccc(Cl)cc2)c2c(=O)[nH]c(N)nc21]

each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

6. The IVT mRNA sequence initiator of clause 1, wherein the initiator has a structure of Formula (I-a):

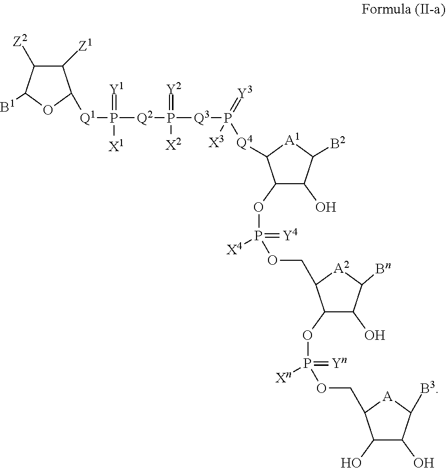

7. The IVT mRNA sequence initiator of clause 1, wherein the initiator has a structure of Formula (I-b):

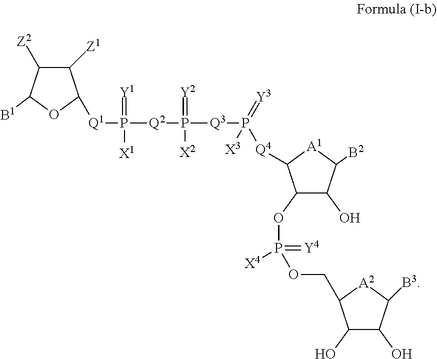

8. The IVT mRNA sequence initiator of clause 1, wherein the initiator has a structure of Formula (I-c):

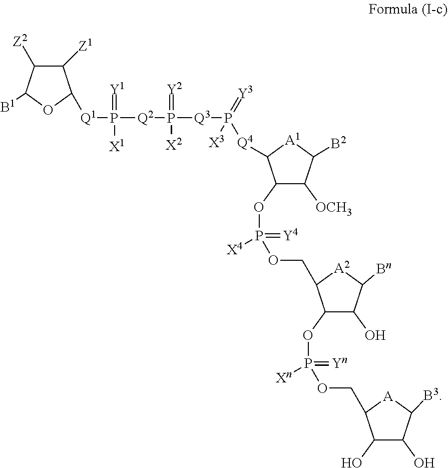

9. The IVT mRNA sequence initiator of clause 1, wherein the initiator has a structure of Formula (I-d):

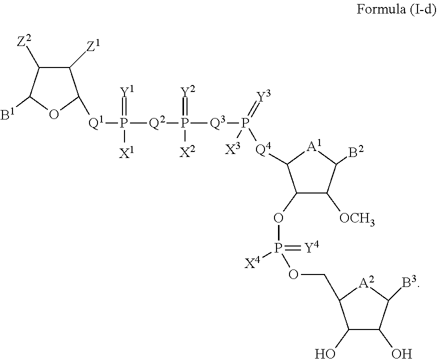

10. The IVT mRNA sequence initiator of any one of clauses 1-5, wherein Z³ is hydrogen, fluorine, —OH, —OCH₃, - or —OCH₂CH₃.

11. The IVT mRNA sequence initiator of clause 10, wherein Z³ is —OCH₃.

12. The IVT mRNA sequence initiator of clause 1-11, wherein each Z⁴ and Zⁿ is independently —OH or —OCH₃.

13. The IVT mRNA sequence initiator of any one of clauses 1-12, wherein each Z³, Z⁴, and Zⁿ is independently —OH or —OCH₃.

14. The IVT mRNA sequence initiator of any one of clauses 1-13, wherein each Y², Y⁴, and Yⁿ is independently ═O or ═S.

15. The IVT mRNA sequence initiator of any one of clauses 1-14, wherein each X² and X³ is independently —O— or —S—.

16. An mRNA sequence having a 5′-end region motif (I′):

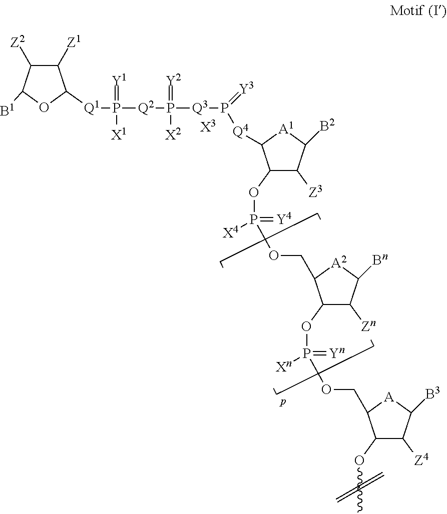

wherein
B¹ is

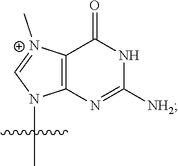 [SMILES: *n1c[n+](C)c2c(=O)[nH]c(N)nc21]

each B², B³, and Bⁿ is independently a natural, a modified, or an unnatural nucleobase;
each Z¹ and Z² is independently hydrogen, fluorine, —OH, —SH, —CH₃, —CH₂CH₃, —OCH₃, —OCH₂CH₃, —SCH₃, —NH₂, NHCH₃, or NHC(═O)CH₃;
each Z³, Z⁴, and Zⁿ is independently hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6,
provided that the compound of Formula (I) satisfies one or more of the following proviso (i) to (iii): (i) at least one of X¹, X², X³, X⁴, and Xⁿ is —SH or —S—; (ii) at least one of Y¹, Y², Y³, Y⁴, and Yⁿ is ═S; and (iii) at least one of A, A¹, and A² is —S—.

17. The mRNA sequence having a 5′-end region motif of clause 16, wherein at least one of X¹, X², X³, X⁴, and Xⁿ is —SH or —S—; at least one of Y¹, Y², Y³, Y⁴, and Yⁿ is ═S; or at least one of A, A¹, and A² is —S—.

18. An mRNA sequence having a 5′end region motif (II′):

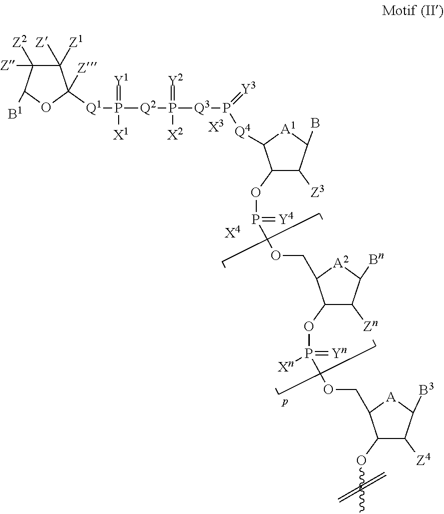

wherein
B¹ is

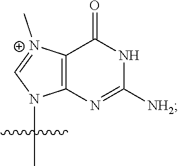 [SMILES: *n1c[n+](C)c2c(=O)[nH]c(N)nc21]

each B², B³, and Bⁿ is independently a natural, a modified, or an unnatural nucleobase;
each Z¹ and Z′ is independently is hydrogen, fluorine, —OH, —SH, —CH₃, —CH₂CH₃, —OCH₃, —NH(CH₃), —NH₂, —NH(C(═O)CH₃), or —SCH₃;
each Z² and Zⁿ is independently fluorine, —OH, —SH, —CH₃, —CH₂CH₃, —OCH₃, —SCH₃, —OCH₂CH₃, —NH₂, NHCH₃, or NHC(═O)CH₃;
Z′″ is hydrogen, fluorine, —CH₃, —CH₂CH₃, —OCH₃, or —OCH₂CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

19. An mRNA sequence having a 5′-end region motif (II′):

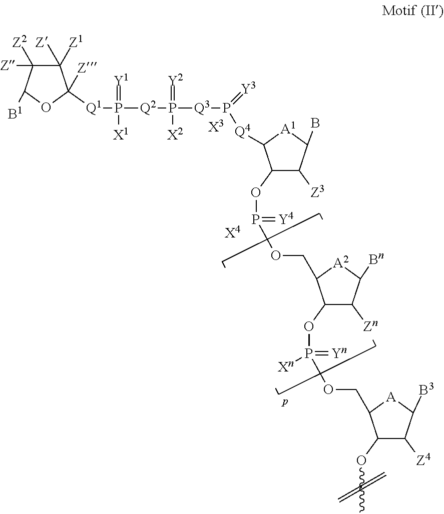

wherein
B¹ is

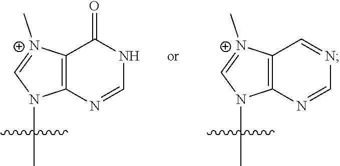 [SMILES: *n1c[n+](C)c2c(=O)[nH]cnc21 ; *n1c[n+](C)c2cncnc21]

each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

20. An mRNA sequence having a 5′-end region motif (II′).

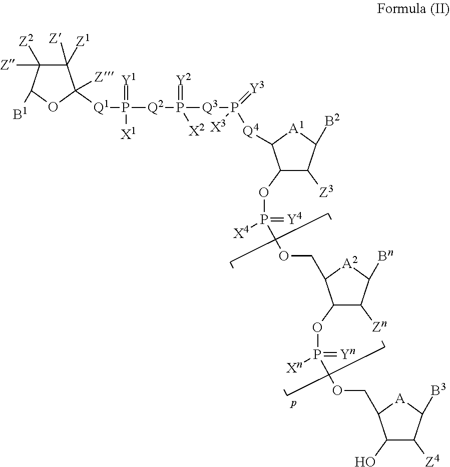

wherein
B¹ is

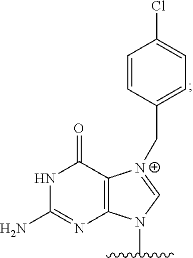 [SMILES: *n1c[n+](Cc2ccc(Cl)cc2)c2c(=O)[nH]c(N)nc21]

each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

21. The mRNA sequence having a 5′-end region motif (I′-a) of clause 16, wherein the motif has a structure of Motif (I′-a):

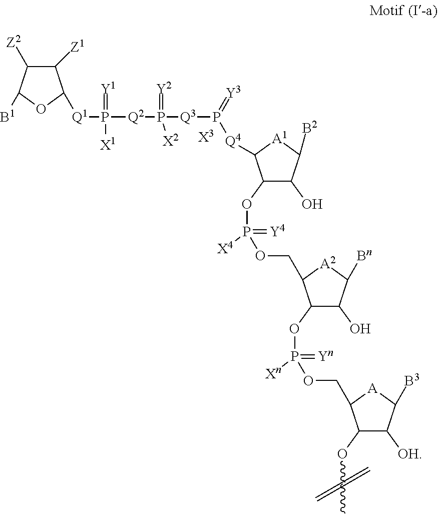

22. The mRNA sequence having a 5′-end region motif (I′-b) of clause 16, wherein the motif has a structure of Motif (I′-b):

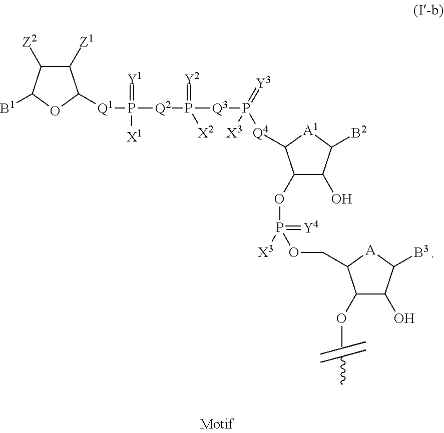

23. The mRNA sequence having a 5′-end region motif (I′-c) of clause 16, wherein the motif has a structure of Motif (I′-c):

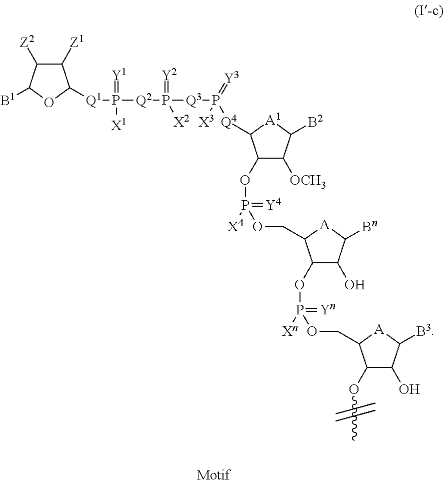

24. The mRNA sequence having a 5′-end region motif (I′-d) of clause 16, wherein the motif has a structure of Motif (I′-d):

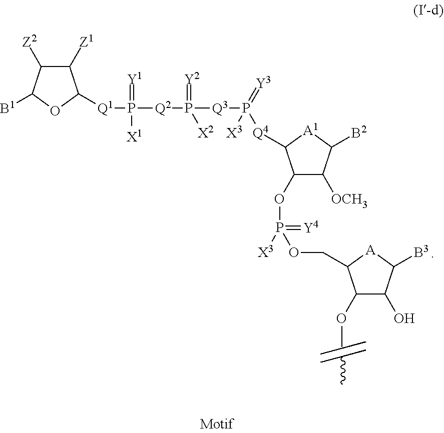

25. The mRNA sequence having a 5′-end region motif (I′-e) of clause 16, wherein the motif has a structure of Motif (I′-e):

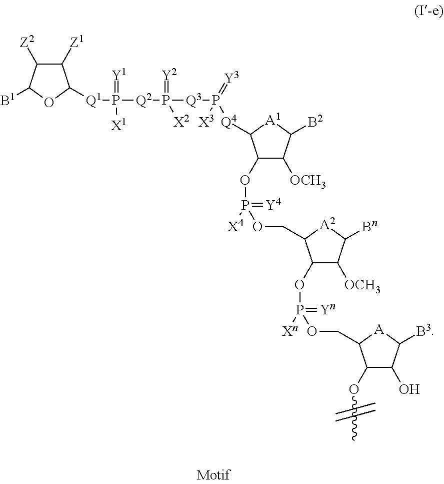

26. The mRNA sequence having a 5′-end region motif (I′-f) of clause 16, wherein the motif has a structure of Motif (I′-f):

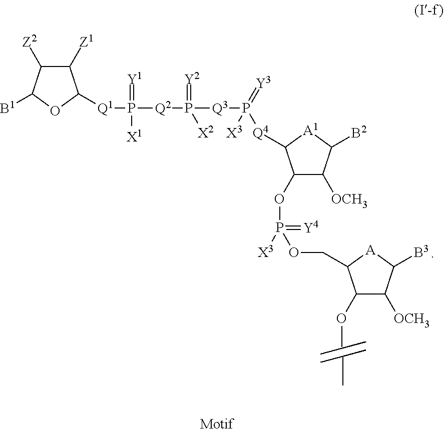

27. The mRNA sequence having a 5′-end region motif (I′-g) of clause 16, wherein the motif has a structure of Motif (I′-g):

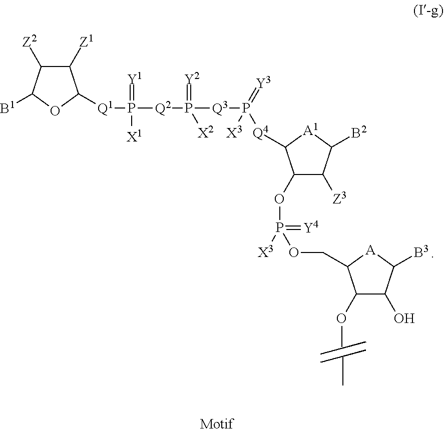

28. The mRNA sequence having a 5′-end region motif (I′-h) of claim 16, wherein the motif has a structure of Motif (I′-h):

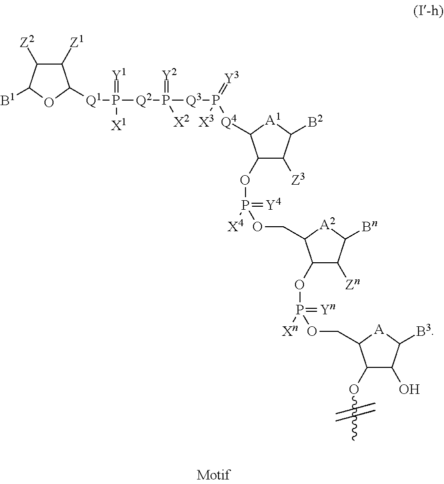

29. The mRNA sequence having a 5′-end region motif (II′-a) of any one of clauses 18-20, wherein the motif has a structure of Motif (II′-a):

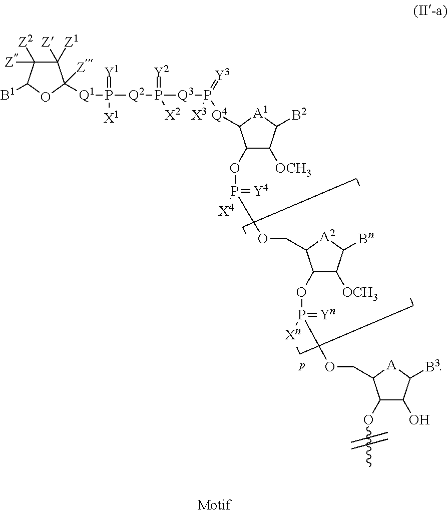

30. The mRNA sequence having a 5′-end region motif (II′-b) of any one of clauses 18-20, wherein the motif has a structure of Motif (II′-b):

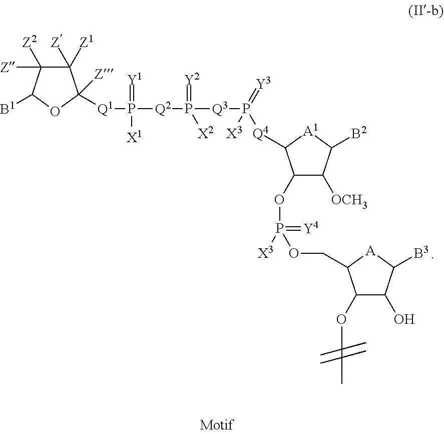

31. The mRNA sequence having a 5′-end region motif (II′-c) of any one of clauses 18-20, wherein the motif has a structure of Motif (II′-c):

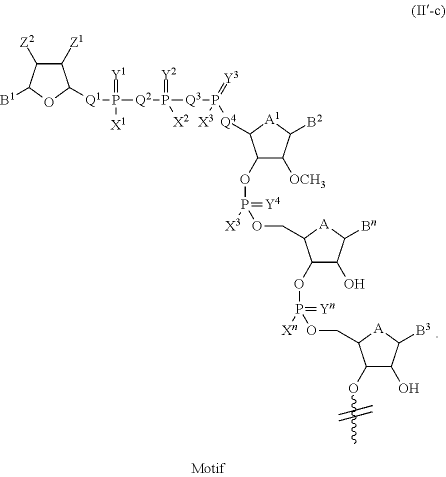

32. The mRNA sequence having a 5′-end region motif (II′-d) of any one of clauses 18-20, wherein the motif has a structure of Motif (II′-d):

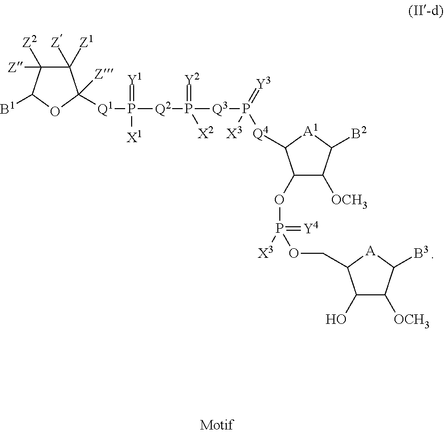

33. The mRNA sequence having a 5′-end region motif (II′-e) of any one of clauses 18-20, wherein the motif has a structure of Motif (II′-e):

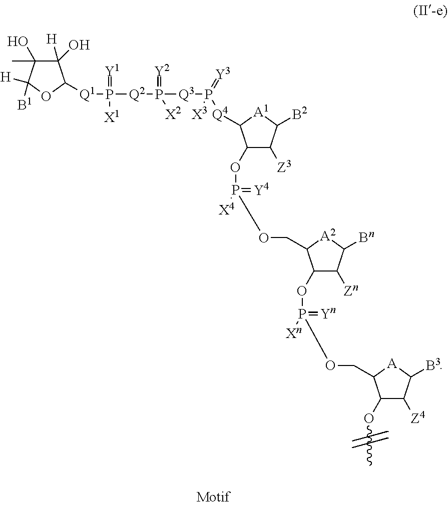

34. The mRNA sequence having a 5′-end region motif (II′-f) of any one of clauses 18-20, wherein the motif has a structure of Motif (II′-f):

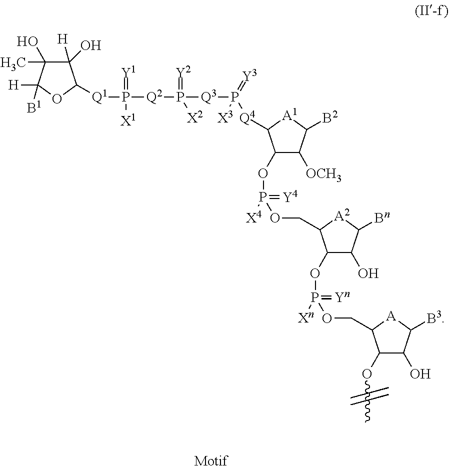

35. The mRNA sequence having a 5′-end region motif (II′-g) of any one of clauses 18-20, wherein the motif has a structure of Motif (II′-g):

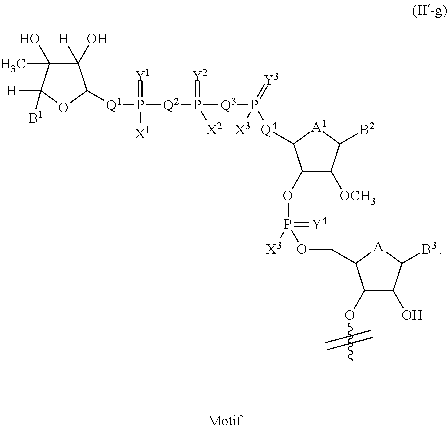

36. The mRNA sequence having a 5′-end region motif (II-h) of any one of clauses 18-20, wherein the motif has a structure of Motif (II′-h):

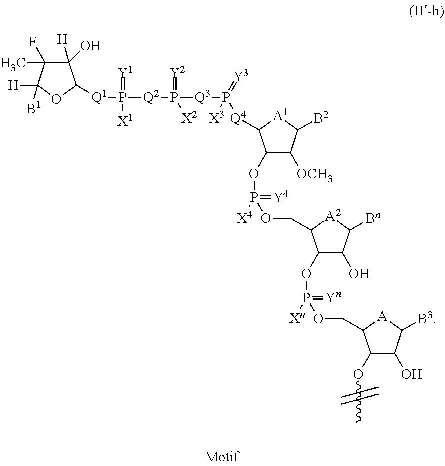

37. The mRNA sequence having a 5′-end region motif (II′-i) of any one of clauses 18-20, wherein the motif has a structure of Motif (II′-i)

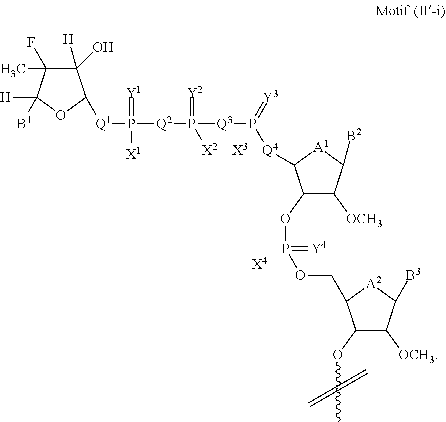

38. The mRNA sequence having a 5′-end region motif (II′-j) of any one of clauses 18-20, wherein the motif has a structure of Motif (II′-j):

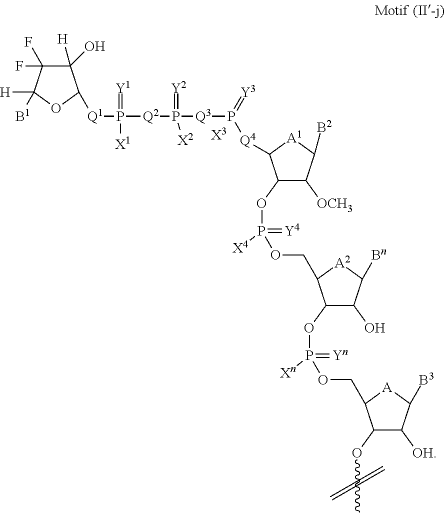

39. The mRNA sequence having a 5′-end region motif (II′-k) of any one of clauses 18-20, wherein the motif has a structure of Motif (II′-k):

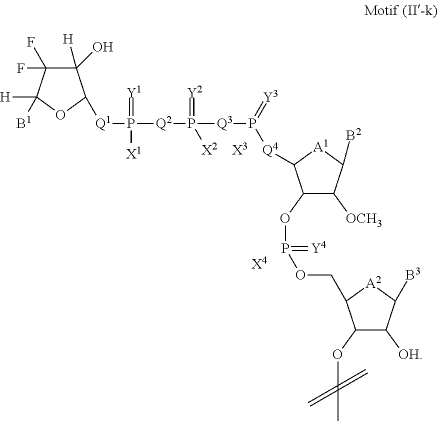

40. The mRNA sequence having a 5′-end region motif (II′-l) of any one of clauses 18-20, wherein the motif has a structure of Motif (II′-l):

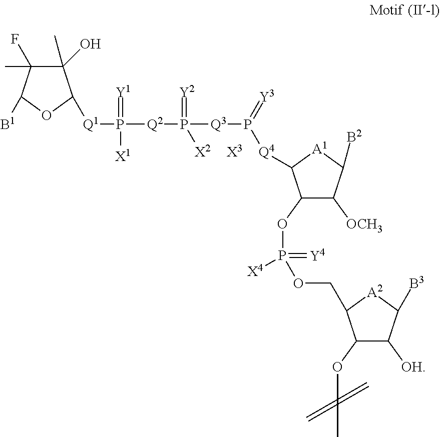

41. The mRNA sequence having a 5′-end region motif (II′-m) of any one of clauses 18-20, wherein the motif has a structure of Motif (II′-m):

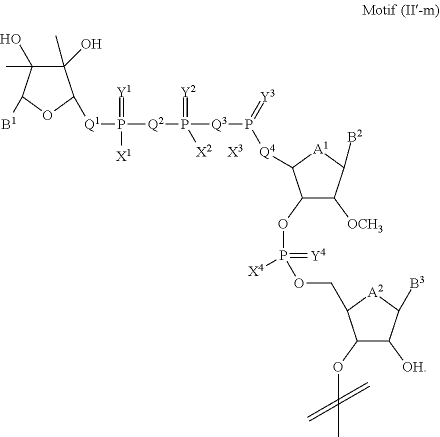

42. The mRNA sequence having a 5′-end region motif (II′-n) of clause 19, wherein the motif has a structure of Motif (II′-n):

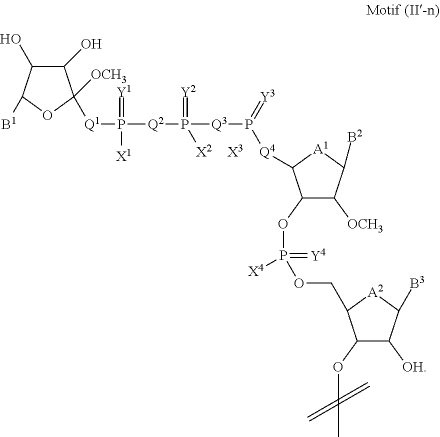

43. The mRNA sequence having a 5′-end region motif (II′-o) of clause 19, wherein the motif has a structure of Motif (II′-o):

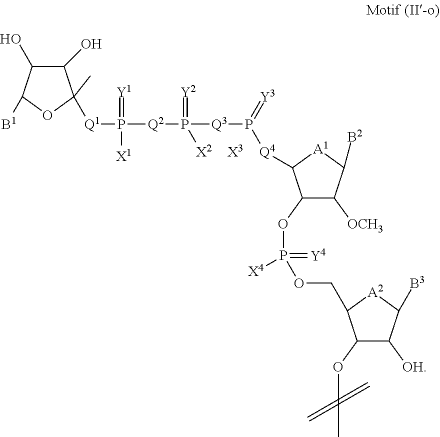

44. The mRNA sequence having a 5′-end region motif (II′-p) of clause 19, wherein the motif has a structure of Motif (II′-p):

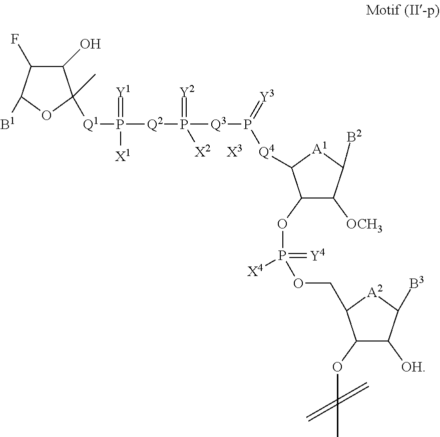

45. The mRNA sequence having a 5′-end region motif (II′-q) of clause 19, wherein the motif has a structure of Motif (II′-q):

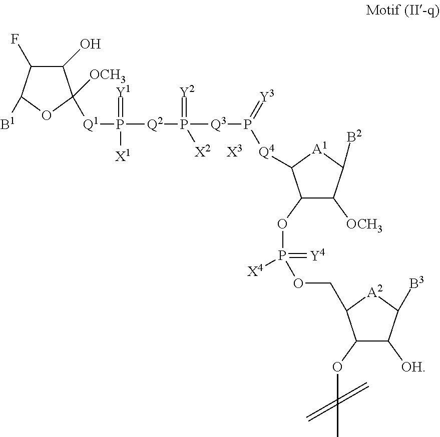

46. The mRNA sequence having a 5′-end region motif (II′-r) of clause 19, wherein the motif has a structure of Motif (II′-r):

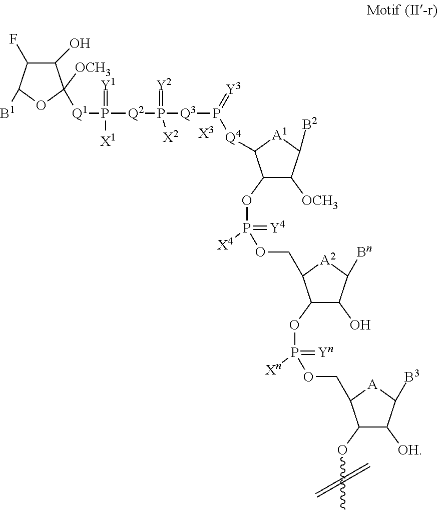

47. The mRNA sequence having a 5′-end region motif (II′-s) of clause 19, wherein the motif has a structure of Motif (II′-s):

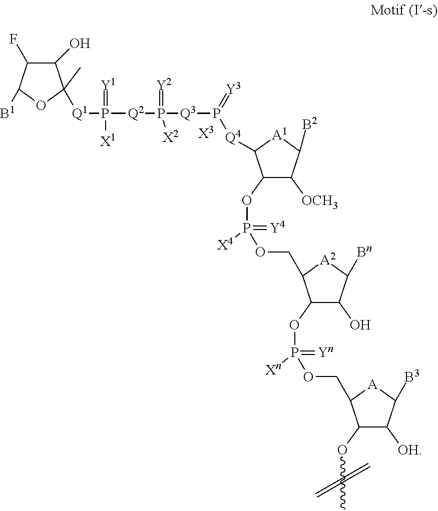

48. The mRNA sequence having a 5′-end region motif (II′-t) of clause 19, wherein the motif has a structure of Motif (II′-t):

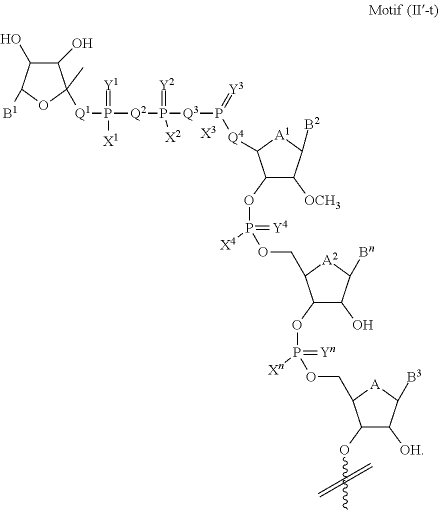

49. The mRNA sequence having a 5′-end region motif (II′-u) of clause 19, wherein the motif has a structure of Motif (II′-u):

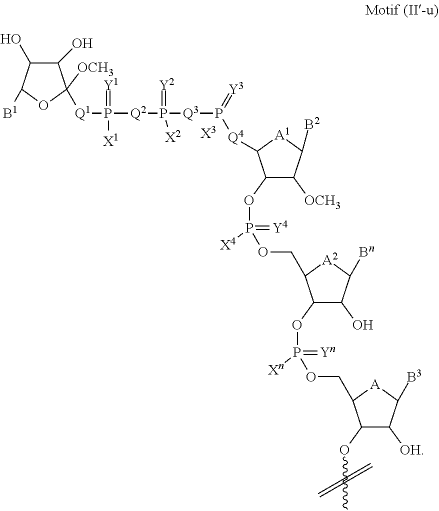

50. The mRNA sequence having a 5′-end region motif of any one of clauses 16-20, wherein Z³ is hydrogen, fluorine, —OH, —OCH₃, - or —OCH₂CH₃.

51. The mRNA sequence having a 5′-end region motif of clause 50, wherein Z³ is —OCH₃.

52. The mRNA sequence having a 5′-end region motif of clause 50, wherein each Z⁴ and Zⁿ is independently —OH or —OCH₃.

53. The mRNA sequence having a 5′-end region motif of any one of clauses 16-20, wherein each Z³, Z⁴, and Zⁿ is independently —OH or —OCH₃.

54. The mRNA sequence having a 5′-end region motif of any one of clauses 19 or 26-52, wherein B¹ is

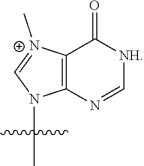 [SMILES: *n1c[n+](C)c2c(=O)[nH]cnc21]

55. The mRNA sequence having a 5′-end region motif of any one of clauses 19 or 26-52, wherein B¹ is

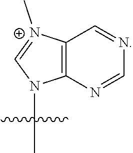 [SMILES: *n1c[n+](C)c2cncnc21]

56. The mRNA sequence having a 5′-end region motif of any one of clauses 19 or 26-52, wherein B¹ is

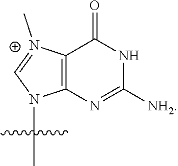 [SMILES: *n1c[n+](C)c2c(=O)[nH]c(N)nc21]

57. The mRNA sequence having a 5′-end region motif of clause 20, wherein B¹ is

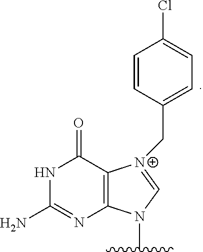 [SMILES: *n1c[n+](Cc2ccc(Cl)cc2)c2c(=O)[nH]c(N)nc21]

58. The mRNA sequence having a 5′-end region motif of any one of clauses 16-32, wherein Z¹ is fluorine, —OH, or —OCH₃.

59. The mRNA sequence having a 5′-end region motif of clause 58, wherein Z¹ is fluorine.

60. The mRNA sequence having a 5′-end region motif of clause 58, wherein Z¹ is —OH.

61. The mRNA sequence having a 5′-end region motif of clause 58, wherein Z¹ is —OCH₃.

62. The mRNA sequence having a 5′-end region motif of any one of clauses 16-32, wherein Z² is fluorine, —OH, or —OCH₃.

63. The mRNA sequence having a 5′-end region motif of clause 62, wherein Z² is fluorine.

64. The mRNA sequence having a 5′-end region motif of clause 62, wherein Z² is —OH.

65. The mRNA sequence having a 5′-end region motif of clause 62, wherein Z² is —OCH₃.

66. The mRNA sequence having a 5′-end region motif of any one of clauses 16-65, wherein Q¹ and Q⁴ is —CH₂O—.

67. The mRNA sequence having a 5′-end region motif of any one of clauses 16-66, wherein each Q² and Q³ is —O—.

68. The mRNA sequence having a 5′-end region motif of any one of clauses 16-67, wherein each Y¹ and Y³ is ═O.

69. The mRNA sequence having a 5′-end region motif of any one of clauses 16-67, wherein each Y², Y⁴, and Yⁿ is independently ═O or ═S.

70. The mRNA sequence having a 5′-end region motif of any one of clauses 16-67, wherein one or more of Y¹, Y², Y³, Y⁴, and Yⁿ is ═S.

71. The mRNA sequence having a 5′-end region motif of clause 70, wherein Y² is ═S.

72. The mRNA sequence having a 5′-end region motif of clause 70, wherein Y⁴ is ═S.

73. The mRNA sequence having a 5′-end region motif of any one of clauses 16-67, wherein each Y¹, Y², Y³, Y⁴, and Yⁿ is ═O.

74. The mRNA sequence having a 5′-end region motif of any one of clauses 16-73, wherein each X¹, X⁴, and Xⁿ is —O⁻.

75. The mRNA sequence having a 5′-end region motif of any one of clauses 16-73, wherein each X² and X³ is independently —O⁻ or —S⁻.

76. The mRNA sequence having a 5′-end region motif of clause 75, wherein X³ is —O⁻.

77. The mRNA sequence having a 5′-end region motif of any one of clauses 16-73, wherein one or more of X¹, X², X³, X⁴, and Xⁿ is —S⁻.

78. The mRNA sequence having a 5′-end region motif of clause 77, wherein X² is —S⁻.

79. The mRNA sequence having a 5′-end region motif of clause 77, wherein X⁴ is —S⁻.

80. The mRNA sequence having a 5′-end region motif of any one of clauses 16-73, wherein each X¹, X², X³, X⁴, and Xⁿ is —O⁻.

81. The mRNA sequence having a 5′-end region motif of any one of clauses 16-80, wherein each A, A¹, and A² is —O—.

82. The mRNA sequence having a 5′-end region motif of any one of clauses 16-80, wherein one or more of A, A¹, and A² is —S—.

83. The mRNA sequence having a 5′-end region motif of any one of clauses 16-80 or 82, wherein A is —S— and A¹ and A² is —O—.

84. The mRNA sequence having a 5′-end region motif of any one of clauses 16-80 or 82, wherein A² is —S— and A and A¹ is —O—.

85. The mRNA sequence having a 5′-end region motif of any one of clauses 16-80, wherein A, A¹, and A² is —O—.

86. The mRNA sequence having a 5′-end region motif of any one of clauses 16, 18, or 50-84, wherein p is 0.

87. The mRNA sequence having a 5′-end region motif of any one of clauses 16, 18, or 50-84, wherein p is 1.

88. The mRNA sequence having a 5′-end region motif of any one of clauses 16, 18, or 50-84, wherein p is 2.

89. The mRNA sequence having a 5′-end region motif of any one of clauses 16-88, wherein each B², B³, and Bⁿ is independently adenine, cytosine, guanine, uracil, thymine, hypoxanthine, or purine.

90. The mRNA sequence having a 5′-end region motif of clause 89, wherein B² is adenine and B³ is guanine.

91. The mRNA sequence having a 5′-end region motif of clause 89, wherein B² is guanine and B³ is adenine.

92. The mRNA sequence having a 5′-end region motif of any one of clauses 16 to 91, wherein protein expression is increased.

93. The mRNA sequence having a 5′-end region motif of clauses 19-49, wherein Q¹ and Q⁴ are —CH₂O—; Q² and Q³ are —O—; each Xⁿ is independently —OH, —SH, O⁻, or S⁻; each Yⁿ is independently ═O or ═S; and B¹ is

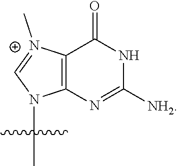 [SMILES: *n1c[n+](C)c2c(=O)[nH]c(N)nc21]

94. The mRNA sequence having a 5′-end region motif of clauses 19-49, wherein Q¹ and Q⁴ are —CH₂O—; Q² and Q³ are —O—; each Xⁿ is independently —OH, —SH, O⁻, or S⁻; each Yⁿ is independently ═O or ═S; and B¹ is

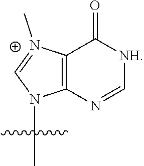 [SMILES: *n1c[n+](C)c2c(=O)[nH]cnc21]

95. The mRNA sequence having a 5′-end region motif of clauses 19-49, wherein Q¹ and Q⁴ are —CH₂O—; Q² and Q³ are —O—; each Xⁿ is independently —OH, —SH, O⁻, or S⁻; each Yⁿ is independently ═O or ═S; and B¹ is

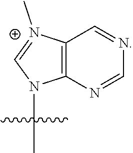 [SMILES: *n1c[n+](C)c2cncnc21]

96. The mRNA sequence having a 5′-end region motif of clauses 89-95, wherein B² is adenine.

97. The mRNA sequence having a 5′-end region motif of clauses 89-95, wherein B³ is guanine.

98. A complex comprising an mRNA sequence having a 5′end region motif and a DNA template, wherein the mRNA sequence having a 5′-end region motif comprises a compound of any one of clauses 16-97, wherein
the DNA template comprises a promoter region comprising a transcriptional start site having a first nucleotide at nucleotide position +1, a second nucleotide at nucleotide position +2, and a third nucleotide at nucleotide position +3; and
wherein the mRNA sequence having a 5′-end region motif is hybridized to the DNA template at least at nucleotide positions +1, +2, and +3.

99. A complex comprising an mRNA sequence having a 5′-end region motif and a DNA template, wherein the mRNA sequence having a 5′end region motif comprises a compound of any one of clauses 16-97, wherein
the DNA template comprises a promoter region comprising a transcriptional start site having a first nucleotide at nucleotide position +1 and a second nucleotide at nucleotide position +2; and
wherein the mRNA sequence having a 5′-end region motif is hybridized to the DNA template at least at nucleotide positions +1 and +2.

100. An RNA molecule comprising the mRNA sequence having a 5′-end region motif according to any one of clauses 16-98.

101. The RNA of clause 100, wherein the RNA comprises a guide RNA or a nuclease mRNA.

102. The RNA of clause 100 or 101, wherein the RNA comprises an mRNA.

103. A method of expressing an mRNA comprising introducing the mRNA of clause 102 into a cell lysate to express the mRNA.

104. The method of clause 103, further comprising measuring the expression level of the mRNA.

105. The method of clause 104, wherein the expression level of the mRNA is at least 2-fold, 3-fold, 4-fold, 6-fold, 8-fold, or 20-fold greater compared to a corresponding mRNA without the mRNA sequence having a 5′-end region motif.

106. The method of clause 104, wherein the cell is a HeLa cell.

107. A method of detecting cellular immune stimulation resulting from mRNA comprising (a) contacting a formulation comprising capped mRNA according to any one of clauses 16 to 97 with a cell reporter line and (b) measuring RIG-I activation in said cell reporter line.

108. The method of clause 107, wherein said cell reporter line is HEK-Lucia RIG-I model.

109. The method of any one of clauses 107 or 108, wherein cellular immune stimulation is reduced compared to an uncapped mRNA by at least 20%.

110. The method of clause 109, wherein cellular immune stimulation is reduced compared to an uncapped mRNA by at least 50%.

111. The method of clause 110, wherein cellular immune stimulation is reduced compared to an uncapped mRNA by at least 70%.

112. The method of clause 111, wherein cellular immune stimulation is reduced compared to an uncapped mRNA by at least 100%.

113. The method of clause 112, wherein cellular immune stimulation is reduced compared to an uncapped mRNA by at least 150%.

114. A method of producing an mRNA sequence having a 5′-end region motif of any one of clauses 16-102 using an IVT reaction comprising (a) mixing a DNA template, polymerase enzyme, mRNA sequence initiator comprising a phosphorothioate group, and nucleoside triphosphates (NTPs) at a specified molar ratio of said mRNA sequence initiator to said NTPs to generate a mixture (b) incubating said mixture at a specified temperature and duration and (c) harvesting and purifying said mRNA sequence having a 5′-end region motif from said mixture.

115. The method of clause 114, wherein said molar ratio of the mRNA sequence initiator to NTPs is about 1:5, and wherein said method is capable of producing a yield of at least 80% with a capping efficiency of at least 80%.

116. The method of clause 114, wherein said molar ratio of the mRNA sequence initiator to NTPs is 1:2.5, and wherein said method is capable of producing a yield of at least 80% with a capping efficiency of at least 85%.

117. The method of clause 114, wherein said molar ratio of the mRNA sequence initiator to NTPs is about 1:1.67, and wherein said method is capable of producing a yield of at least 80% with a capping efficiency of at least 90%.

118. The method of clause 114, wherein said molar ratio of the mRNA sequence initiator to NTPs is about 1:1.25, and wherein said method is capable of producing a yield of at least 80% with a capping efficiency of at least 90%.

119. The method of clause 114, wherein said molar ratio of the mRNA sequence initiator to NTPs is about 1:1, and wherein said method is capable of producing a yield of at least 80% with a capping efficiency of at least 90%.

120. The method of clause 114, wherein said molar ratio of the mRNA sequence initiator to NTPs is about 1:5, and wherein said method is capable of producing a yield of at least 3 mg of mRNA per milliliter (mL) of IVT reaction with a capping efficiency of at least 80%.

121. The method of clause 114, wherein said molar ratio of the mRNA sequence initiator to NTPs is about 1:2.5, and wherein said method is capable of producing a yield of at least 3 mg of mRNA per mL of IVT reaction with a capping efficiency of at least 85%.

122. The method of clause 114, wherein said molar ratio of the mRNA sequence initiator to NTPs is about 1:1.67, and wherein said method is capable of producing a yield of at least 3 mg of mRNA per mL of IVT reaction with a capping efficiency of at least 90%.

123. The method of clause 114, wherein said molar ratio of the mRNA sequence initiator to NTPs is about 1:1.25, and wherein said method is capable of producing a yield of at least 3 mg of mRNA per mL of IVT reaction with a capping efficiency of at least 90%.

124. The method of clause 114, wherein said molar ratio of the mRNA sequence initiator to NTPs is about 1:1, and wherein said method is capable of producing a yield of at least 3 mg of mRNA per mL of IVT reaction with a capping efficiency of at least 90%.

125. The method of clause 114, wherein said NTPs is GTP, ATP, CTP, UTP, or a modified NTP, or a combination thereof.

126. The method of clause 125, wherein said modified NTP is 5′-triphosphate of N1-methyl pseudouridine.

127. A cell containing an RNA molecule comprising the mRNA sequence having a 5′ end region motif according to any one of clauses 16-97.

128. A cell containing a polypeptide or protein translated from an RNA molecule comprising the mRNA sequence having a 5′end region motif according to any one of clauses 16-97.

129. A pharmaceutical composition comprising an RNA molecule comprising the mRNA sequence having a 5′end region motif according to any one of clauses 16-97 and one or more of pharmaceutically acceptable excipients.

130. The pharmaceutical composition of clause 129, further comprising one or more single guide RNA designed to target one or more specific locations of one or more genes of interest to elicit pharmacological effect upon administration into a mammal.

131. The pharmaceutical composition of clause 129, comprising lipid nanoparticles.

132. The pharmaceutical composition of clause 130, wherein the RNA is encapsulated in a lipid nanoparticle.

133. A method for synthesizing an RNA molecule comprising:
a. introducing the mRNA sequence having a 5′end region motif according to any one of clauses 16-98 into a mixture comprising an RNA polymerase, and
b. incubating the mixture for a time sufficient to allow for transcription of the RNA molecule.

134. The method of clause 133 wherein the mixture further comprises a DNA template and nucleoside triophosphates.

135. A method of gene editing comprising introducing into a cell an RNA molecule of clause 100 or 102, or pharmaceutical composition of any one of clauses 130-133, wherein the RNA molecule comprises guide RNA or a nuclease mRNA, wherein the RNA molecule is translated in the cell.

136. A method for reducing the risk of coronary disease in a subject in need thereof, comprising administering to the subject an effective amount of a pharmaceutical composition of any one of clauses 130-133.

1. An in vitro-transcribed (IVT) mRNA sequence initiator comprising a compound of Formula (I) or a salt or solvate thereof: 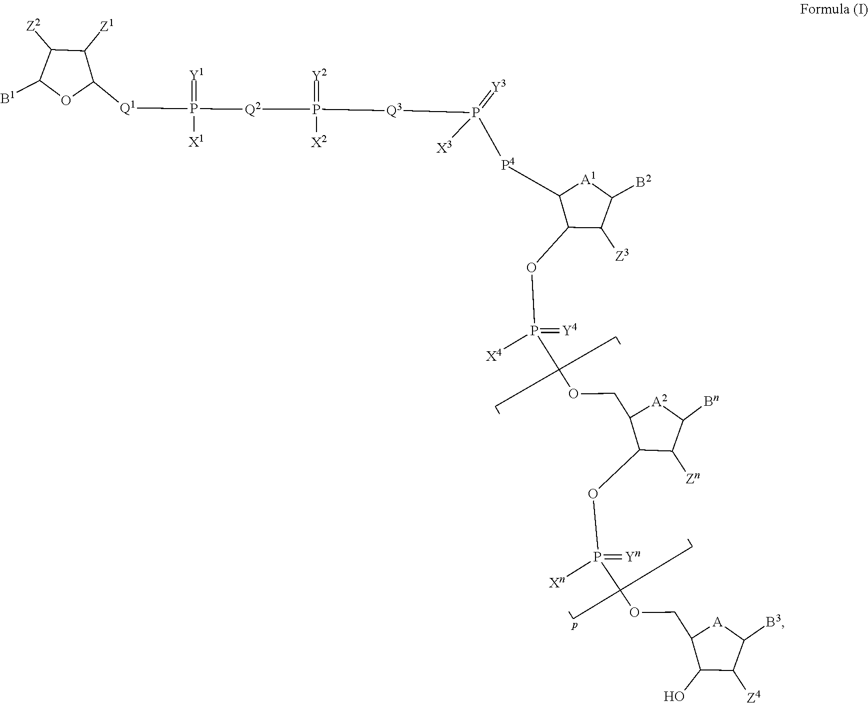
wherein
B¹ is 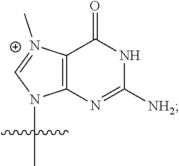 [SMILES: *n1c[n+](C)c2c(=O)[nH]c(N)nc21]
each B², B³, and Bⁿ is independently a natural, a modified, or an unnatural nucleobase;
each Z¹ and Z² is independently hydrogen, fluorine, —OH, —SH, —CH₃, —CH₂CH₃, —OCH₃, —OCH₂CH₃, —SCH₃, —NH₂, NHCH₃, or NHC(═O)CH₃;
Z³ is —OCH₃;
each Z⁴, and Zⁿ is independently hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
X⁴ is —OH or —O⁻;
each X¹, X², X³, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃;
Y⁴ is ═S;
each Y¹, Y², Y³ and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6,
wherein the compound comprises a phosphorothioate, and where the phosphorothioate comprises a chiral phosphorous center.

2.-4. (canceled)

5. An in vitro-transcribed (IVT) mRNA sequence initiator comprising a compound of Formula (II) or a salt or solvate thereof: 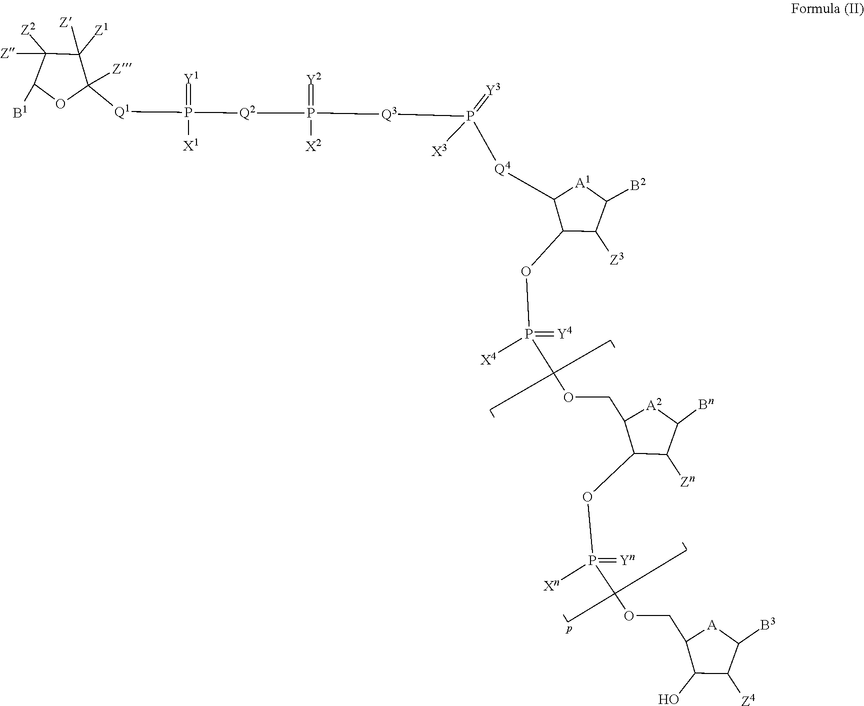
wherein
B¹ is 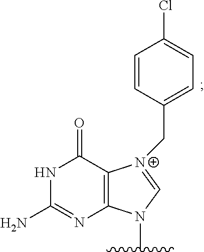 [SMILES: *n1c[n+](Cc2ccc(Cl)cc2)c2c(=O)[nH]c(N)nc21]
each B², B³, and Bⁿ is independently a natural, a modified, or an unnatural nucleobase;
each Z′ and Z″ is independently is hydrogen, fluorine, —OH, —SH, —CH₃, —CH₂CH₃, —OCH₃, —NH(CH₃), —NH₂, —NH(C(═O)CH₃), or —SCH₃;
Z′″ is hydrogen, fluorine, —CH₃, —CH₂CH₃, —OCH₃, or —OCH₂CH₃;
each Z¹ and Z² is independently hydrogen, fluorine, —OH, —SH, —CH₃, —CH₂CH₃, —OCH₃, —SCH₃, —OCH₂CH₃, —NH₂, NHCH₃, or NHC(═O)CH₃;
each Z³, Z⁴, and Zⁿ is independently hydrogen, fluorine, —OH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH═CH—, —CH₂—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃ or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

6. An in vitro-transcribed (IVT) mRNA sequence initiator comprising a compound of Formula (III) or a salt or solvate thereof: 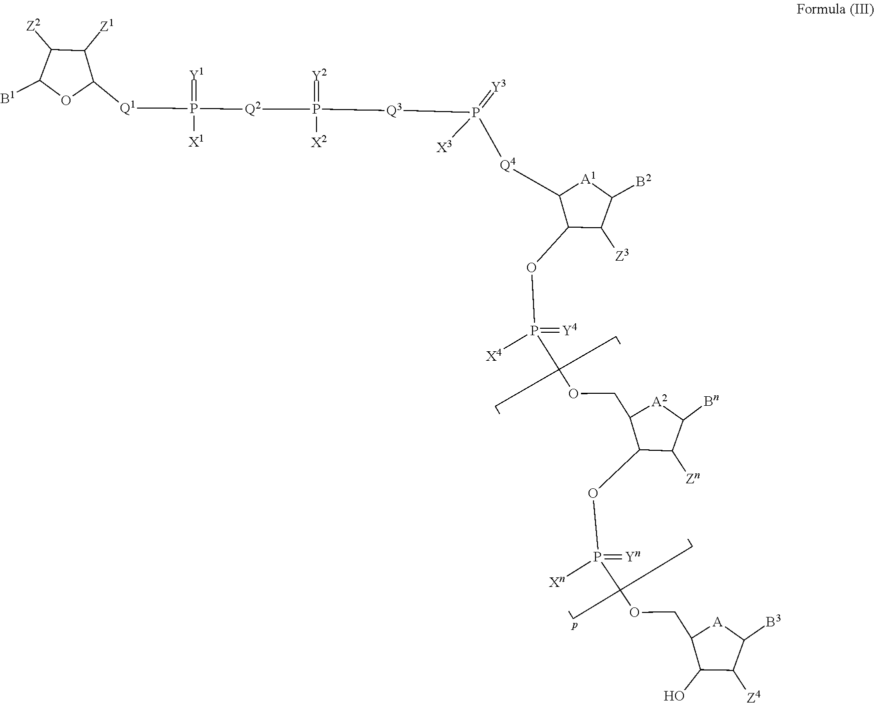
wherein
B¹ is 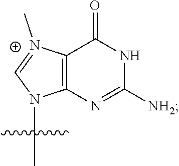 [SMILES: *n1c[n+](C)c2c(=O)[nH]c(N)nc21]
B² is a modified or unnatural nucleobase;
each B³, and Bⁿ is independently a natural, a modified, or an unnatural nucleobase;
each Z¹ and Z² is independently hydrogen, fluorine, —OH, —SH, —CH₃, —CH₂CH₃, —OCH₃, —OCH₂CH₃, —SCH₃, —NH₂, NHCH₃, or NHC(═O)CH₃;
each Z³, Z⁴, and Zⁿ is independently hydrogen, fluorine, —OH, —SH, —CH₃, —CH₂CH₃, —OCH₃, —NH₂, —NHCH₃, —NH(C(═O)CH₃), —OCH₂CH₃, —OCH₂OCH₃, —OCH₂CH₂CH₃, —OCH(CH₃)₂, —SCH₃, or —OCH₂CH₂OCH₃;
each Q¹ and Q⁴ is independently —CH₂—, —CH═CH—, —CH₂O—, —CH₂S—, —CH₂CH₂—, —CH₂CF₂—, —CH₂NH₂—, —CH₂NH(CH₃)—, or —CH₂N(C(═O)CH₃)—;
each Q² and Q³ is independently —O—, —S—, —CH₂—, —CF₂—, —NH—, —N(CH₃)—, or —N(C(═O)CH₃)—;
each X¹, X², X³, X⁴, and Xⁿ is independently —OH, —SH, —O⁻, —S⁻, —NH₂, —NHCH₃, —NH(C(═O)CH₃), —CH₃, —CH₂CH₃, —CH₂CH₂CH₃, —CH(CH₃)₂, —OCH₃, or —OCH₂CH₃;
each Y¹, Y², Y³, Y⁴, and Yⁿ is independently ═O, ═S, ═NH, or ═NCH₃;
each A, A¹, and A² is independently —O—, —S—, —CH₂—, —NH—, —N(CH₃)— or —N(C(═O)CH₃)—; and
p=0, 1, 2, 3, 4, 5 or 6.

7.-8. (canceled)

9. The IVT mRNA sequence initiator of claim 1, wherein the initiator has a structure of Formula (I-c) or Formula (I-d):

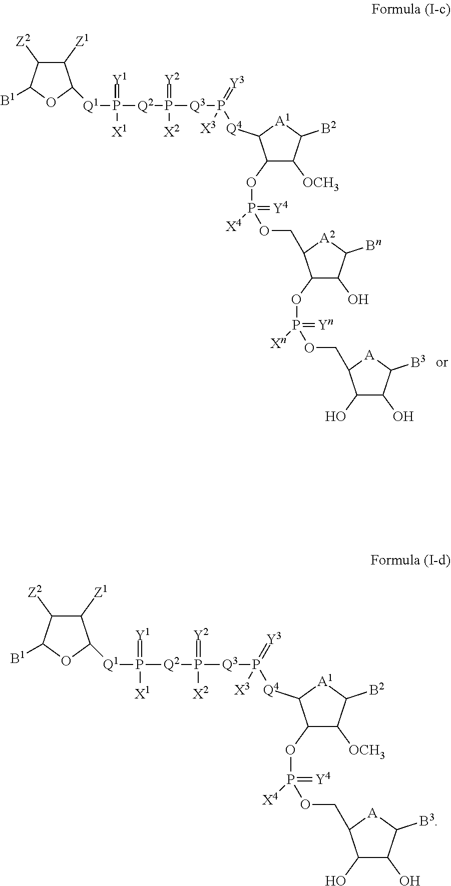

10. (canceled)

11. The IVT mRNA sequence initiator of claim 6, wherein the initiator has a structure of Formula (III-a):

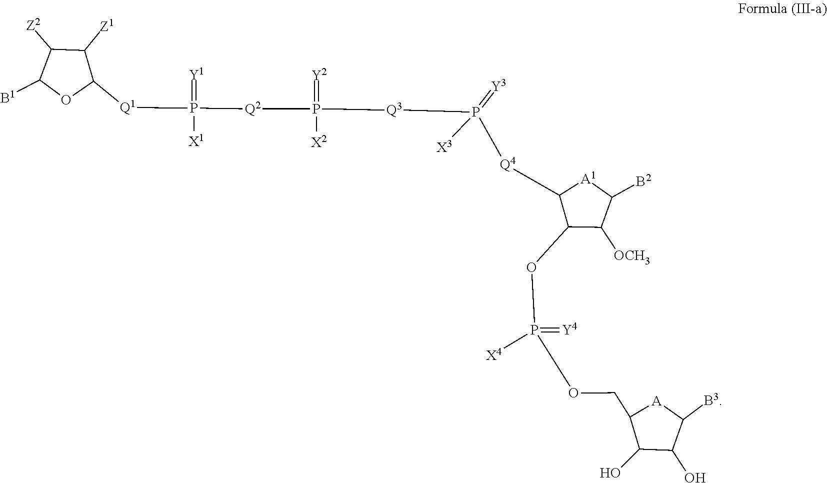

12.-13. (canceled)

14. The IVT mRNA sequence initiator of claim 1, wherein
each Z⁴ and Zⁿ is independently —OH or —OCH₃,
each Y¹, Y², Y³, and Yⁿ is independently ═O or ═S,
each X¹, X², X³ and Xⁿ is independently —O⁻ or —S⁻.

15.-17. (canceled)

18. The IVT mRNA sequence initiator of claim 6, wherein B₂ is

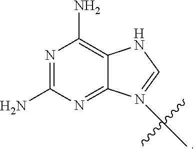

19. The IVT mRNA sequence initiator of claim 6, wherein the compound is

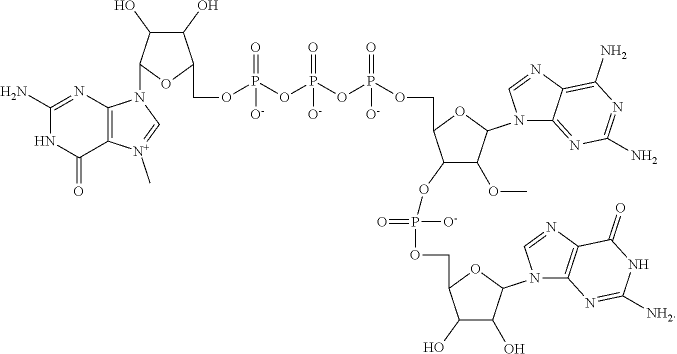 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)nc(N)nc21]

20. The IVT mRNA sequence initiator of claim 5, wherein the compound is

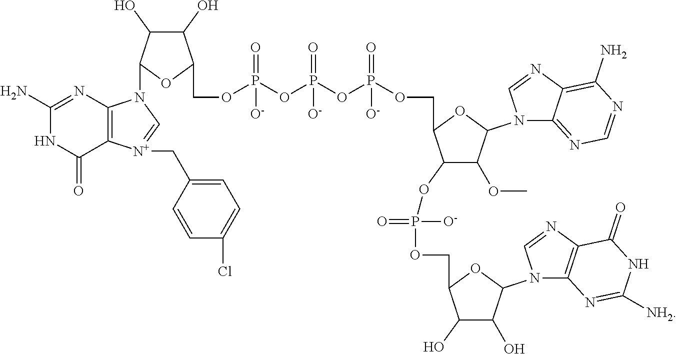 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](Cc4ccc(Cl)cc4)c4c(=O)[nH]c(N)nc43)C(O)C2O)OC1n1cnc2c(N)ncnc21]

21.-23. (canceled)

24. The IVT mRNA sequence initiator of claim 1, wherein the compound is

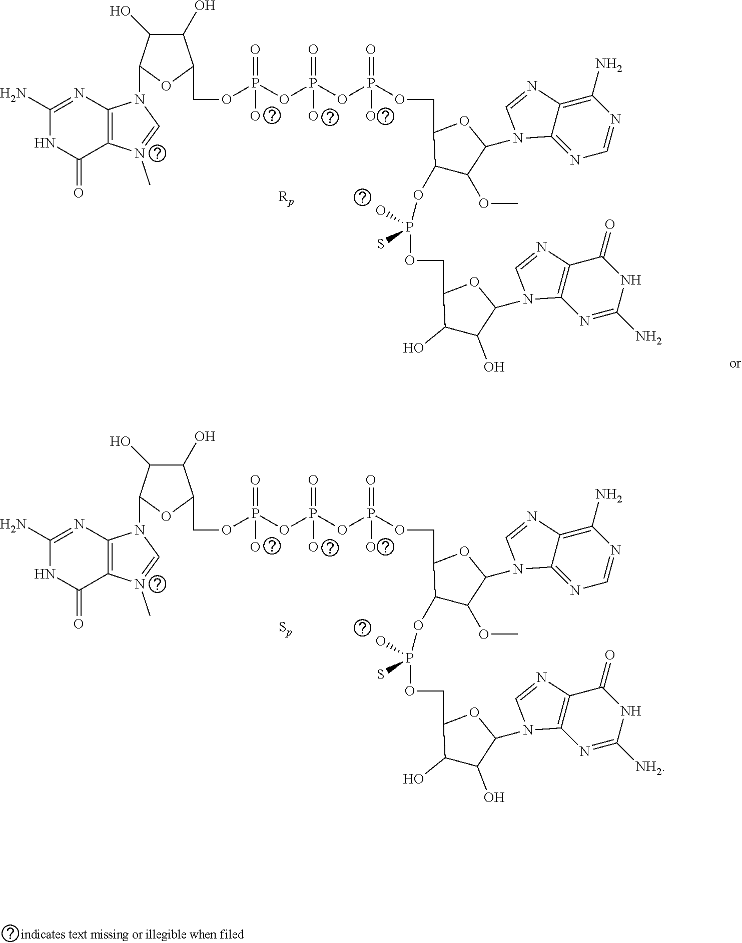

25.-31. (canceled)

32. An mRNA sequence comprising the IVT mRNA sequence initiator of claim 1.

33.-35. (canceled)

36. An mRNA sequence comprising the IVT mRNA sequence initiator of claim 5.

37. An mRNA sequence comprising the IVT mRNA sequence initiator of claim 6.

38.-162. (canceled)

163. A pharmaceutical composition comprising the mRNA sequence of claim 32 and one or more of pharmaceutically acceptable excipients.

164.-169. (canceled)

170. A method for reducing the risk of coronary disease in a subject in need thereof, comprising administering to the subject an effective amount of a pharmaceutical composition of claim 163.

171. The IVT mRNA sequence initiator of claim 9, wherein
B² is adenine and B³ is guanine;
each of Q¹ and Q⁴ is —CH₂O—;
each of Q² and Q³ is —O—;
each of X¹, X², and X³ is —OH or —O⁻;
each of Y¹, Y², and Y³ is ═O; and
each of A¹ and A is —O—.

172. The mRNA sequence of claim 32, wherein the mRNA sequence comprises
(a) a 5′ untranslated region,
(b) a first region encoding a deaminase,
(c) a second region encoding a programmable nuclease,
(d) a third region encoding a nuclear localization sequence,
(e) a 3′ untranslated region, and
(f) a polyadenylic acid region.

173. The mRNA sequence of claim 172, wherein
the deaminase is an adenine base editor, and
the programmable nuclease is a Cas9 protein.

174. The mRNA sequence of claim 32, wherein the mRNA sequence comprises a nucleotide sequence having at least 90% sequence identity to SEQ ID NO: 3 or 4.

175. A method of producing an mRNA sequence using an IVT reaction comprising
(a) mixing a DNA template, a polymerase enzyme, the IVT mRNA sequence initiator of claim 1, and nucleoside triphosphates (NTPs) at a specified molar ratio of the IVT mRNA sequence initiator to the NTPs to generate a mixture;
(b) incubating the mixture at a specified temperature and duration; and
(c) harvesting and purifying the mRNA sequence from the mixture,
wherein said molar ratio of the IVT mRNA sequence initiator to the NTPs is about 1:5, about 1:2.5, about 1:1.67, about 1:1.25, or about 1:1;
wherein said method is capable of producing a yield of
(i) at least 80%, or
(ii) at least 3 mg of mRNA per mL of the IVT reaction,
with a capping efficiency of at least 80%; and
wherein said NTPs is GTP, ATP, CTP, UTP, or a modified NTP, or a combination thereof.

176. A pharmaceutical composition comprising the mRNA sequence of claim 36 and one or more of pharmaceutically acceptable excipients.

177. A method for reducing the risk of coronary disease in a subject in need thereof, comprising administering to the subject an effective amount of a pharmaceutical composition of claim 176.

178. The mRNA sequence of claim 36, wherein the mRNA sequence comprises
(a) a 5′ untranslated region,
(b) a first region encoding a deaminase,
(c) a second region encoding a programmable nuclease,
(d) a third region encoding a nuclear localization sequence,
(e) a 3′ untranslated region, and
(f) a polyadenylic acid region.

179. The mRNA sequence of claim 178, wherein
the deaminase is an adenine base editor, and
the programmable nuclease is a Cas9 protein.

180. The mRNA sequence of claim 36, wherein the mRNA sequence comprises a nucleotide sequence having at least 90% sequence identity to SEQ ID NO: 3 or 4.

181. A method of producing an mRNA sequence using an IVT reaction comprising
(a) mixing a DNA template, a polymerase enzyme, the IVT mRNA sequence initiator of claim 5, and nucleoside triphosphates (NTPs) at a specified molar ratio of the IVT mRNA sequence initiator to the NTPs to generate a mixture;
(b) incubating the mixture at a specified temperature and duration; and
(c) harvesting and purifying the mRNA sequence from the mixture,
wherein said molar ratio of the IVT mRNA sequence initiator to the NTPs is about 1:5, about 1:2.5, about 1:1.67, about 1:1.25, or about 1:1;
wherein said method is capable of producing a yield of
(i) at least 80%, or
(ii) at least 3 mg of mRNA per mL of the IVT reaction,
with a capping efficiency of at least 80%; and
wherein said NTPs is GTP, ATP, CTP, UTP, or a modified NTP, or a combination thereof.

182. A pharmaceutical composition comprising the mRNA sequence of claim 37 and one or more of pharmaceutically acceptable excipients.

183. A method for reducing the risk of coronary disease in a subject in need thereof, comprising administering to the subject an effective amount of a pharmaceutical composition of claim 182.

184. The mRNA sequence of claim 37, wherein the mRNA sequence comprises
(a) a 5′ untranslated region,
(b) a first region encoding a deaminase,
(c) a second region encoding a programmable nuclease,
(d) a third region encoding a nuclear localization sequence,
(e) a 3′ untranslated region, and
(f) a polyadenylic acid region.

185. The mRNA sequence of claim 184, wherein
the deaminase is an adenine base editor, and
the programmable nuclease is a Cas9 protein.

186. The mRNA sequence of claim 37, wherein the mRNA sequence comprises a nucleotide sequence having at least 90% sequence identity to SEQ ID NO: 3 or 4.

187. A method of producing an mRNA sequence using an IVT reaction comprising
(a) mixing a DNA template, a polymerase enzyme, the IVT mRNA sequence initiator of claim 6, and nucleoside triphosphates (NTPs) at a specified molar ratio of the IVT mRNA sequence initiator to the NTPs to generate a mixture;
(b) incubating the mixture at a specified temperature and duration; and
(c) harvesting and purifying the mRNA sequence from the mixture,
wherein said molar ratio of the IVT mRNA sequence initiator to the NTPs is about 1:5, about 1:2.5, about 1:1.67, about 1:1.25, or about 1:1;
wherein said method is capable of producing a yield of
(i) at least 80%, or
(ii) at least 3 mg of mRNA per mL of the IVT reaction,
with a capping efficiency of at least 80%; and
wherein said NTPs is GTP, ATP, CTP, UTP, or a modified NTP, or a combination thereof.


SEQUENCE LISTING (4 sequences)

SEQ ID NO: 1 - RNA, 23 nt, synthetic construct
  source 1..23: mol_type = other RNA
taatacgactcactatagggaga

SEQ ID NO: 2 - RNA, 23 nt, synthetic construct
  source 1..23: mol_type = other RNA
tctccctatagtgagtcgtatta

SEQ ID NO: 3 - RNA, 4929 nt, synthetic construct
  source 1..4929: mol_type = other RNA
  modified_base 7: mod_base = OTHER; note = N1-methylpseudouridine
  modified_base 25: mod_base = OTHER; note = N1-methylpseudouridine
  modified_base 35: mod_base = OTHER; note = N1-methylpseudouridine
  modified_base 37: mod_base = OTHER; note = N1-methylpseudouridine
  modified_base 49: mod_base = OTHER; note = N1-methylpseudouridine
  modified_base 58: mod_base = OTHER; note = N1-methylpseudouridine
  modified_base 63: mod_base = OTHER; note = N1-methylpseudouridine
  modified_base 64: mod_base = OTHER; note = N1-methylpseudouridine
  modified_base 75: mod_base = OTHER; note = N1-methylpseudouridine
  modified_base 78: mod_base = OTHER; note = N1-methylpseudouridine
  modified_base 82: mod_base = OTHER; note = N1-methylpseudouridine
  modified_base 94: mod_base = OTHER; note = N1-methylpseudouridine
  modified_base 100: mod_base = OTHER; note = N1-methylpseudouridine
  modified_base 130: mod_base = OTHER; note = N1-methylpseudouridine
  modified_base 136: mod_base = OTHER; note = N1-methylpseudouridine
  modified_base 145: mod_base = OTHER; note = N1-methylpseudouridine
  modified_base 148: mod_base = OTHER; note = N1-methylpseudouridine
  modified_base 151: mod_base = OTHER; note = N1-methylpseudouridine
  modified_base 154: mod_base = OTHER; note = N1-methylpseudouridine
  modified_base 166: mod_base = OTHER; note = N1-methylpseudouridine
  modified_base 169: mod_base = OTHER; note = N1-methylpseudouridine
  modified_base 180: mod_base = OTHER; note = N1-methylpseudouridine
  modified_base 193: mod_base = OTHER; note = N1-methylpseudouridine
  modified_base 199: mod_base = OTHER; note = N1-methylpseudouridine
  modified_base 226: mod_base = OTHER; note = N1-methylpseudouridine
  modified_base 229: mod_base = OTHER; note = N1-methylpseudouridine
  modified_base 235: mod_base = OTHER; note = N1-methylpseudouridine
  modified_base 250: mod_base = OTHER; note = N1-methylpseudouridine
  modified_base 253: mod_base = OTHER; note = N1-methylpseudouridine
  modified_base 256: mod_base = OTHER; note = N1-methylpseudouridine
  modified_base 264: mod_base = OTHER; note = N1-methylpseudouridine
  modified_base 271: mod_base = OTHER; note = N1-methylpseudouridine
  modified_base 274: mod_base = OTHER; note = N1-methylpseudouridine
  modified_base 286: mod_base = OTHER; note = N1-methylpseudouridine
  modified_base 288: mod_base = OTHER; note = N1-methylpseudouridine
  modified_base 292: mod_base = OTHER; note = N1-methylpseudouridine
  modified_base 297: mod_base = OTHER; note = N1-methylpseudouridine
  modified_base 298: mod_base = OTHER; note = N1-methylpseudouridine
  modified_base 306: mod_base = OTHER; note = N1-methylpseudouridine
  modified_base 310: mod_base = OTHER; note = N1-methylpseudouridine
  modified_base 313: mod_base = OTHER; note = N1-methylpseudouridine
  modified_base 315: mod_base = OTHER; note = N1-methylpseudouridine
  modified_base 328: mod_base = OTHER; note = N1-methylpseudouridine
  modified_base 331: mod_base = OTHER; note = N1-methylpseudouridine
  modified_base 343: mod_base = OTHER; note = N1-methylpseudouridine
  modified_base 352: mod_base = OTHER; note = N1-methylpseudouridine
  modified_base 355: mod_base = OTHER; note = N1-methylpseudouridine
  modified_base 357: mod_base = OTHER; note = N1-methylpseudouridine
  modified_base 358: mod_base = OTHER; note = N1-methylpseudouridine
  modified_base 364: mod_base = OTHER; note = N1-methylpseudouridine
  modified_base 397: mod_base = OTHER; note = N1-methylpseudouridine
  modified_base 400: mod_base = OTHER; note = N1-methylpseudouridine
  modified_base 406: mod_base = OTHER; note = N1-methylpseudouridine
  modified_base 409: mod_base = OTHER; note = N1-methylpseudouridine
  modified_base 424: mod_base = OTHER; note = N1-methylpseudouridine
  modified_base 436: mod_base = OTHER; note = N1-methylpseudouridine
  modified_base 442: mod_base = OTHER; note = N1-methylpseudouridine
  modified_base 454: mod_base = OTHER; note = N1-methylpseudouridine
  modified_base 457: mod_base = OTHER; note = N1-methylpseudouridine
  modified_base 468: mod_base = OTHER; note = N1-methylpseudouridine
  modified_base 478: mod_base = OTHER; note = N1-methylpseudouridine
  modified_base 481: mod_base = OTHER; note = N1-methylpseudouridine
  modified_base 483: mod_base = OTHER; note = N1-methylpseudouridine
  modified_base 489: mod_base = OTHER; note = N1-methylpseudouridine
  modified_base 490: mod_base = OTHER; note = N1-methylpseudouridine
  modified_base 492: mod_base = OTHER; note = N1-methylpseudouridine
  modified_base 493: mod_base = OTHER; note = N1-methylpseudouridine
  modified_base 499: mod_base = OTHER; note = N1-methylpseudouridine
  modified_base 511: mod_base = OTHER; note = N1-methylpseudouridine
  modified_base 513: mod_base = OTHER; note = N1-methylpseudouridine
  modified_base 514: mod_base = OTHER; note = N1-methylpseudouridine
  modified_base 654: mod_base = OTHER; note = N1-methylpseudouridine
  modified_base 661: mod_base = OTHER; note = N1-methylpseudouridine
  modified_base 667: mod_base = OTHER; note = N1-methylpseudouridine
  modified_base 673: mod_base = OTHER; note = N1-methylpseudouridine
  modified_base 688: mod_base = OTHER; note = N1-methylpseudouridine
  modified_base 693: mod_base = OTHER; note = N1-methylpseudouridine
  modified_base 700: mod_base = OTHER; note = N1-methylpseudouridine
  modified_base 703: mod_base = OTHER; note = N1-methylpseudouridine
  modified_base 714: mod_base = OTHER; note = N1-methylpseudouridine
  modified_base 721: mod_base = OTHER; note = N1-methylpseudouridine
  modified_base 735: mod_base = OTHER; note = N1-methylpseudouridine
  modified_base 736: mod_base = OTHER; note = N1-methylpseudouridine
  modified_base 742: mod_base = OTHER; note = N1-methylpseudouridine
  modified_base 745: mod_base = OTHER; note = N1-methylpseudouridine
  modified_base 769: mod_base = OTHER; note = N1-methylpseudouridine
  modified_base 781: mod_base = OTHER; note = N1-methylpseudouridine
  modified_base 784: mod_base = OTHER; note = N1-methylpseudouridine
  modified_base 793: mod_base = OTHER; note = N1-methylpseudouridine
  modified_base 796: mod_base = OTHER; note = N1-methylpseudouridine
  modified_base 798: mod_base = OTHER; note = N1-methylpseudouridine
  modified_base 799: mod_base = OTHER; note = N1-methylpseudouridine
  modified_base 832: mod_base = OTHER; note = N1-methylpseudouridine
  modified_base 855: mod_base = OTHER; note = N1-methylpseudouridine
  modified_base 877: mod_base = OTHER; note = N1-methylpseudouridine
  modified_base 879: mod_base = OTHER; note = N1-methylpseudouridine
  modified_base 882: mod_base = OTHER; note = N1-methylpseudouridine
  modified_base 886: mod_base = OTHER; note = N1-methylpseudouridine
  modified_base 895: mod_base = OTHER; note = N1-methylpseudouridine
  modified_base 897: mod_base = OTHER; note = N1-methylpseudouridine
  modified_base 898: mod_base = OTHER; note = N1-methylpseudouridine
  modified_base 910: mod_base = OTHER; note = N1-methylpseudouridine
  modified_base 919: mod_base = OTHER; note = N1-methylpseudouridine
  modified_base 930: mod_base = OTHER; note = N1-methylpseudouridine
  modified_base 931: mod_base = OTHER; note = N1-methylpseudouridine
  modified_base 933: mod_base = OTHER; note = N1-methylpseudouridine
  modified_base 934: mod_base = OTHER; note = N1-methylpseudouridine
  modified_base 943: mod_base = OTHER; note = N1-methylpseudouridine
  modified_base 954: mod_base = OTHER; note = N1-methylpseudouridine
  modified_base 955: mod_base = OTHER; note = N1-methylpseudouridine
  modified_base 958: mod_base = OTHER; note = N1-methylpseudouridine
  modified_base 961: mod_base = OTHER; note = N1-methylpseudouridine
  modified_base 994: mod_base = OTHER; note = N1-methylpseudouridine
  modified_base 996: mod_base = OTHER; note = N1-methylpseudouridine
  modified_base 997: mod_base = OTHER; note = N1-methylpseudouridine
  modified_base 1006: mod_base = OTHER; note = N1-methylpseudouridine
  modified_base 1009: mod_base = OTHER; note = N1-methylpseudouridine
  modified_base 1018: mod_base = OTHER; note = N1-methylpseudouridine
  modified_base 1023: mod_base = OTHER; note = N1-methylpseudouridine
  modified_base 1035: mod_base = OTHER; note = N1-methylpseudouridine
  modified_base 1045: mod_base = OTHER; note = N1-methylpseudouridine
  modified_base 1047: mod_base = OTHER; note = N1-methylpseudouridine
  modified_base 1054: mod_base = OTHER; note = N1-methylpseudouridine
  modified_base 1066: mod_base = OTHER; note = N1-methylpseudouridine
  modified_base 1069: mod_base = OTHER; note = N1-methylpseudouridine
  modified_base 1093: mod_base = OTHER; note = N1-methylpseudouridine
  modified_base 1099: mod_base = OTHER; note = N1-methylpseudouridine
  modified_base 1102: mod_base = OTHER; note = N1-methylpseudouridine
  modified_base 1104: mod_base = OTHER; note = N1-methylpseudouridine
  modified_base 1108: mod_base = OTHER; note = N1-methylpseudouridine
  modified_base 1114: mod_base = OTHER; note = N1-methylpseudouridine
  modified_base 1123: mod_base = OTHER; note = N1-methylpseudouridine
  modified_base 1126: mod_base = OTHER; note = N1-methylpseudouridine
  modified_base 1131: mod_base = OTHER; note = N1-methylpseudouridine
  modified_base 1132: mod_base = OTHER; note = N1-methylpseudouridine
  modified_base 1143: mod_base = OTHER; note = N1-methylpseudouridine
  modified_base 1144: mod_base = OTHER; note = N1-methylpseudouridine
  modified_base 1147: mod_base = OTHER; note = N1-methylpseudouridine
  modified_base 1150: mod_base = OTHER; note = N1-methylpseudouridine
  modified_base 1162: mod_base = OTHER; note = N1-methylpseudouridine
  modified_base 1183: mod_base = OTHER; note = N1-methylpseudouridine
  modified_base 1192: mod_base = OTHER; note = N1-methylpseudouridine
  modified_base 1194: mod_base = OTHER; note = N1-methylpseudouridine
  modified_base 1195: mod_base = OTHER; note = N1-methylpseudouridine
  modified_base 1198: mod_base = OTHER; note = N1-methylpseudouridine
  modified_base 1204: mod_base = OTHER; note = N1-methylpseudouridine
  modified_base 1207: mod_base = OTHER; note = N1-methylpseudouridine
  modified_base 1215: mod_base = OTHER; note = N1-methylpseudouridine
  modified_base 1225: mod_base = OTHER; note = N1-methylpseudouridine
  modified_base 1227: mod_base = OTHER; note = N1-methylpseudouridine
  modified_base 1228: mod_base = OTHER; note = N1-methylpseudouridine
  modified_base 1243: mod_base = OTHER; note = N1-methylpseudouridine
  modified_base 1258: mod_base = OTHER; note = N1-methylpseudouridine
  modified_base 1273: mod_base = OTHER; note = N1-methylpseudouridine
  modified_base 1276: mod_base = OTHER; note = N1-methylpseudouridine
  modified_base 1288: mod_base = OTHER; note = N1-methylpseudouridine
  modified_base 1306: mod_base = OTHER; note = N1-methylpseudouridine
  modified_base 1315: mod_base = OTHER; note = N1-methylpseudouridine
  modified_base 1318: mod_base = OTHER; note = N1-methylpseudouridine
  modified_base 1327: mod_base = OTHER; note = N1-methylpseudouridine
  modified_base 1351: mod_base = OTHER; note = N1-methylpseudouridine
  modified_base 1353: mod_base = OTHER; note = N1-methylpseudouridine
  modified_base 1354: mod_base = OTHER; note = N1-methylpseudouridine
  modified_base 1363: mod_base = OTHER; note = N1-methylpseudouridine
  modified_base 1366: mod_base = OTHER; note = N1-methylpseudouridine
  modified_base 1372: mod_base = OTHER; note = N1-methylpseudouridine
  modified_base 1378: mod_base = OTHER; note = N1-methylpseudouridine
  modified_base 1384: mod_base = OTHER; note = N1-methylpseudouridine
  modified_base 1395: mod_base = OTHER; note = N1-methylpseudouridine
  modified_base 1396: mod_base = OTHER; note = N1-methylpseudouridine
  modified_base 1407: mod_base = OTHER; note = N1-methylpseudouridine
  modified_base 1408: mod_base = OTHER; note = N1-methylpseudouridine
  modified_base 1414: mod_base = OTHER; note = N1-methylpseudouridine
  modified_base 1432: mod_base = OTHER; note = N1-methylpseudouridine
  modified_base 1438: mod_base = OTHER; note = N1-methylpseudouridine
  modified_base 1452: mod_base = OTHER; note = N1-methylpseudouridine
  modified_base 1465: mod_base = OTHER; note = N1-methylpseudouridine
  modified_base 1474: mod_base = OTHER; note = N1-methylpseudouridine
  modified_base 1477: mod_base = OTHER; note = N1-methylpseudouridine
  modified_base 1486: mod_base = OTHER; note = N1-methylpseudouridine
  modified_base 1497: mod_base = OTHER; note = N1-methylpseudouridine
  modified_base 1507: mod_base = OTHER; note = N1-methylpseudouridine
  modified_base 1509: mod_base = OTHER; note = N1-methylpseudouridine
  modified_base 1510: mod_base = OTHER; note = N1-methylpseudouridine
  modified_base 1513: mod_base = OTHER; note = N1-methylpseudouridine
  modified_base 1528: mod_base = OTHER; note = N1-methylpseudouridine
  modified_base 1540: mod_base = OTHER; note = N1-methylpseudouridine
  modified_base 1543: mod_base = OTHER; note = N1-methylpseudouridine
  modified_base 1546: mod_base = OTHER; note = N1-methylpseudouridine
  modified_base 1555: mod_base = OTHER; note = N1-methylpseudouridine
  modified_base 1558: mod_base = OTHER; note = N1-methylpseudouridine
  modified_base 1564: mod_base = OTHER; note = N1-methylpseudouridine
  modified_base 1576: mod_base = OTHER; note = N1-methylpseudouridine
  modified_base 1591: mod_base = OTHER; note = N1-methylpseudouridine
  modified_base 1603: mod_base = OTHER; note = N1-methylpseudouridine
  modified_base 1606: mod_base = OTHER; note = N1-methylpseudouridine
  modified_base 1614: mod_base = OTHER; note = N1-methylpseudouridine
  modified_base 1636: mod_base = OTHER; note = N1-methylpseudouridine
  modified_base 1642: mod_base = OTHER; note = N1-methylpseudouridine
  modified_base 1645: mod_base = OTHER; note = N1-methylpseudouridine
  modified_base 1654: mod_base = OTHER; note = N1-methylpseudouridine
  modified_base 1657: mod_base = OTHER; note = N1-methylpseudouridine
  modified_base 1669: mod_base = OTHER; note = N1-methylpseudouridine
  modified_base 1680: mod_base = OTHER; note = N1-methylpseudouridine
  modified_base 1690: mod_base = OTHER; note = N1-methylpseudouridine
  modified_base 1692: mod_base = OTHER; note = N1-methylpseudouridine
  modified_base 1693: mod_base = OTHER; note = N1-methylpseudouridine
  modified_base 1695: mod_base = OTHER; note = N1-methylpseudouridine
  modified_base 1696: mod_base = OTHER; note = N1-methylpseudouridine
  modified_base 1716: mod_base = OTHER; note = N1-methylpseudouridine
  modified_base 1725: mod_base = OTHER; note = N1-methylpseudouridine
  modified_base 1729: mod_base = OTHER; note = N1-methylpseudouridine
  modified_base 1755: mod_base = OTHER; note = N1-methylpseudouridine
  modified_base 1756: mod_base = OTHER; note = N1-methylpseudouridine
  modified_base 1758: mod_base = OTHER; note = N1-methylpseudouridine
  modified_base 1764: mod_base = OTHER; note = N1-methylpseudouridine
  modified_base 1765: mod_base = OTHER; note = N1-methylpseudouridine
  modified_base 1768: mod_base = OTHER; note = N1-methylpseudouridine
  modified_base 1777: mod_base = OTHER; note = N1-methylpseudouridine
  modified_base 1780: mod_base = OTHER; note = N1-methylpseudouridine
  modified_base 1789: mod_base = OTHER; note = N1-methylpseudouridine
  modified_base 1807: mod_base = OTHER; note = N1-methylpseudouridine
  modified_base 1810: mod_base = OTHER; note = N1-methylpseudouridine
  modified_base 1813: mod_base = OTHER; note = N1-methylpseudouridine
  modified_base 1819: mod_base = OTHER; note = N1-methylpseudouridine
  modified_base 1834: mod_base = OTHER; note = N1-methylpseudouridine
  modified_base 1837: mod_base = OTHER; note = N1-methylpseudouridine
  modified_base 1854: mod_base = OTHER; note = N1-methylpseudouridine
  modified_base 1855: mod_base = OTHER; note = N1-methylpseudouridine
  modified_base 1870: mod_base = OTHER; note = N1-methylpseudouridine
  modified_base 1882: mod_base = OTHER; note = N1-methylpseudouridine
  modified_base 1888: mod_base = OTHER; note = N1-methylpseudouridine
  modified_base 1897: mod_base = OTHER; note = N1-methylpseudouridine
  modified_base 1906: mod_base = OTHER; note = N1-methylpseudouridine
  modified_base 1909: mod_base = OTHER; note = N1-methylpseudouridine
  modified_base 1926: mod_base = OTHER; note = N1-methylpseudouridine
  modified_base 1927: mod_base = OTHER; note = N1-methylpseudouridine
  modified_base 1929: mod_base = OTHER; note = N1-methylpseudouridine
  modified_base 1935: mod_base = OTHER; note = N1-methylpseudouridine
  modified_base 1936: mod_base = OTHER; note = N1-methylpseudouridine
  modified_base 1939: mod_base = OTHER; note = N1-methylpseudouridine
  modified_base 1960: mod_base = OTHER; note = N1-methylpseudouridine
  modified_base 1969: mod_base = OTHER; note = N1-methylpseudouridine
  modified_base 1972: mod_base = OTHER; note = N1-methylpseudouridine
  modified_base 1977: mod_base = OTHER; note = N1-methylpseudouridine
  modified_base 1978: mod_base = OTHER; note = N1-methylpseudouridine
  modified_base 1984: mod_base = OTHER; note = N1-methylpseudouridine
  modified_base 1989: mod_base = OTHER; note = N1-methylpseudouridine
  modified_base 1992: mod_base = OTHER; note = N1-methylpseudouridine
  modified_base 1996: mod_base = OTHER; note = N1-methylpseudouridine
  modified_base 2005: mod_base = OTHER; note = N1-methylpseudouridine
  modified_base 2025: mod_base = OTHER; note = N1-methylpseudouridine
  modified_base 2026: mod_base = OTHER; note = N1-methylpseudouridine
  modified_base 2031: mod_base = OTHER; note = N1-methylpseudouridine
  modified_base 2035: mod_base = OTHER; note = N1-methylpseudouridine
  modified_base 2059: mod_base = OTHER; note = N1-methylpseudouridine
  modified_base 2067: mod_base = OTHER; note = N1-methylpseudouridine
  modified_base 2073: mod_base = OTHER; note = N1-methylpseudouridine
  modified_base 2074: mod_base = OTHER; note = N1-methylpseudouridine
  modified_base 2083: mod_base = OTHER; note = N1-methylpseudouridine
  modified_base 2086: mod_base = OTHER; note = N1-methylpseudouridine
  modified_base 2112: mod_base = OTHER; note = N1-methylpseudouridine
  modified_base 2113: mod_base = OTHER; note = N1-methylpseudouridine
  modified_base 2116: mod_base = OTHER; note = N1-methylpseudouridine
  modified_base 2125: mod_base = OTHER; note = N1-methylpseudouridine
  modified_base 2133: mod_base = OTHER; note = N1-methylpseudouridine
  modified_base 2134: mod_base = OTHER; note = N1-methylpseudouridine
  modified_base 2146: mod_base = OTHER; note = N1-methylpseudouridine
  modified_base 2161: mod_base = OTHER; note = N1-methylpseudouridine
  modified_base 2164: mod_base = OTHER; note = N1-methylpseudouridine
  modified_base 2179: mod_base = OTHER; note = N1-methylpseudouridine
  modified_base 2182: mod_base = OTHER; note = N1-methylpseudouridine
  modified_base 2184: mod_base = OTHER; note = N1-methylpseudouridine
  modified_base 2190: mod_base = OTHER; note = N1-methylpseudouridine
  modified_base 2193: mod_base = OTHER; note = N1-methylpseudouridine
  modified_base 2194: mod_base = OTHER; note = N1-methylpseudouridine
  modified_base 2200: mod_base = OTHER; note = N1-methylpseudouridine
  modified_base 2202: mod_base = OTHER; note = N1-methylpseudouridine
  modified_base 2212: mod_base = OTHER; note = N1-methylpseudouridine
  modified_base 2221: mod_base = OTHER; note = N1-methylpseudouridine
  modified_base 2226: mod_base = OTHER; note = N1-methylpseudouridine
  modified_base 2230: mod_base = OTHER; note = N1-methylpseudouridine
  modified_base 2242: mod_base = OTHER; note = N1-methylpseudouridine
  modified_base 2256: mod_base = OTHER; note = N1-methylpseudouridine
  modified_base 2257: mod_base = OTHER; note = N1-methylpseudouridine
  modified_base 2260: mod_base = OTHER; note = N1-methylpseudouridine
  modified_base 2284: mod_base = OTHER; note = N1-methylpseudouridine
  modified_base 2287: mod_base = OTHER; note = N1-methylpseudouridine
  modified_base 2293: mod_base = OTHER; note = N1-methylpseudouridine
  modified_base 2296: mod_base = OTHER; note = N1-methylpseudouridine
  modified_base 2298: mod_base = OTHER; note = N1-methylpseudouridine
  modified_base 2299: mod_base = OTHER; note = N1-methylpseudouridine
  modified_base 2317: mod_base = OTHER; note = N1-methylpseudouridine
  modified_base 2323: mod_base = OTHER; note = N1-methylpseudouridine
  modified_base 2332: mod_base = OTHER; note = N1-methylpseudouridine
  modified_base 2343: mod_base = OTHER; note = N1-methylpseudouridine
  modified_base 2346: mod_base = OTHER; note = N1-methylpseudouridine
  modified_base 2347: mod_base = OTHER; note = N1-methylpseudouridine
  modified_base 2356: mod_base = OTHER; note = N1-methylpseudouridine
  modified_base 2361: mod_base = OTHER; note = N1-methylpseudouridine
  modified_base 2364: mod_base = OTHER; note = N1-methylpseudouridine
  modified_base 2365: mod_base = OTHER; note = N1-methylpseudouridine
  modified_base 2374: mod_base = OTHER; note = N1-methylpseudouridine
  modified_base 2380: mod_base = OTHER; note = N1-methylpseudouridine
  modified_base 2389: mod_base = OTHER; note = N1-methylpseudouridine
  modified_base 2400: mod_base = OTHER; note = N1-methylpseudouridine
  modified_base 2401: mod_base = OTHER; note = N1-methylpseudouridine
  modified_base 2413: mod_base = OTHER; note = N1-methylpseudouridine
  modified_base 2421: mod_base = OTHER; note = N1-methylpseudouridine
  modified_base 2431: mod_base = OTHER; note = N1-methylpseudouridine
  modified_base 2434: mod_base = OTHER; note = N1-methylpseudouridine
  modified_base 2440: mod_base = OTHER; note = N1-methylpseudouridine
  modified_base 2443: mod_base = OTHER; note = N1-methylpseudouridine
  modified_base 2457: mod_base = OTHER; note = N1-methylpseudouridine
  modified_base 2458: mod_base = OTHER; note = N1-methylpseudouridine
  modified_base 2461: mod_base = OTHER; note = N1-methylpseudouridine
  modified_base 2485: mod_base = OTHER; note = N1-methylpseudouridine
  modified_base 2488: mod_base = OTHER; note = N1-methylpseudouridine
  modified_base 2497: mod_base = OTHER; note = N1-methylpseudouridine
  modified_base 2500: mod_base = OTHER; note = N1-methylpseudouridine
  modified_base 2503: mod_base = OTHER; note = N1-methylpseudouridine
  modified_base 2509: mod_base = OTHER; note = N1-methylpseudouridine
  modified_base 2515: mod_base = OTHER; note = N1-methylpseudouridine
  modified_base 2517: mod_base = OTHER; note = N1-methylpseudouridine
  modified_base 2518: mod_base = OTHER; note = N1-methylpseudouridine
  modified_base 2533: mod_base = OTHER; note = N1-methylpseudouridine
  modified_base 2536: mod_base = OTHER; note = N1-methylpseudouridine
  modified_base 2548: mod_base = OTHER; note = N1-methylpseudouridine
  modified_base 2556: mod_base = OTHER; note = N1-methylpseudouridine
  modified_base 2566: mod_base = OTHER; note = N1-methylpseudouridine
  modified_base 2568: mod_base = OTHER; note = N1-methylpseudouridine
  modified_base 2569: mod_base = OTHER; note = N1-methylpseudouridine
  modified_base 2581: mod_base = OTHER; note = N1-methylpseudouridine
  modified_base 2584: mod_base = OTHER; note = N1-methylpseudouridine
  modified_base 2593: mod_base = OTHER; note = N1-methylpseudouridine
  modified_base 2607: mod_base = OTHER; note = N1-methylpseudouridine
  modified_base 2616: mod_base = OTHER; note = N1-methylpseudouridine
  modified_base 2626: mod_base = OTHER; note = N1-methylpseudouridine
  modified_base 2638: mod_base = OTHER; note = N1-methylpseudouridine
  modified_base 2641: mod_base = OTHER; note = N1-methylpseudouridine
  modified_base 2650: mod_base = OTHER; note = N1-methylpseudouridine
  modified_base 2677: mod_base = OTHER; note = N1-methylpseudouridine
  modified_base 2680: mod_base = OTHER; note = N1-methylpseudouridine
  modified_base 2685: mod_base = OTHER; note = N1-methylpseudouridine
  modified_base 2686: mod_base = OTHER; note = N1-methylpseudouridine
  modified_base 2689: mod_base = OTHER; note = N1-methylpseudouridine
  modified_base 2703: mod_base = OTHER; note = N1-methylpseudouridine
  modified_base 2704: mod_base = OTHER; note = N1-methylpseudouridine
  modified_base 2718: mod_base = OTHER; note = N1-methylpseudouridine
  modified_base 2719: mod_base = OTHER; note = N1-methylpseudouridine
  modified_base 2722: mod_base = OTHER; note = N1-methylpseudouridine
  modified_base 2728: mod_base = OTHER; note = N1-methylpseudouridine
  modified_base 2731: mod_base = OTHER; note = N1-methylpseudouridine
  modified_base 2746: mod_base = OTHER; note = N1-methylpseudouridine
  modified_base 2751: mod_base = OTHER; note = N1-methylpseudouridine
  modified_base 2752: mod_base = OTHER; note = N1-methylpseudouridine
  modified_base 2764: mod_base = OTHER; note = N1-methylpseudouridine
  modified_base 2779: mod_base = OTHER; note = N1-methylpseudouridine
  modified_base 2800: mod_base = OTHER; note = N1-methylpseudouridine
  modified_base 2812: mod_base = OTHER; note = N1-methylpseudouridine
  modified_base 2821: mod_base = OTHER; note = N1-methylpseudouridine
  modified_base 2839: mod_base = OTHER; note = N1-methylpseudouridine
  modified_base 2851: mod_base = OTHER; note = N1-methylpseudouridine
  modified_base 2854: mod_base = OTHER; note = N1-methylpseudouridine
  modified_base 2863: mod_base = OTHER; note = N1-methylpseudouridine
  modified_base 2869: mod_base = OTHER; note = N1-methylpseudouridine
  modified_base 2872: mod_base = OTHER; note = N1-methylpseudouridine
  modified_base 2881: mod_base = OTHER; note = N1-methylpseudouridine
  modified_base 2884: mod_base = OTHER; note = N1-methylpseudouridine
  modified_base 2890: mod_base = OTHER; note = N1-methylpseudouridine
  modified_base 2893: mod_base = OTHER; note = N1-methylpseudouridine
  modified_base 2917: mod_base = OTHER; note = N1-methylpseudouridine
  modified_base 2920: mod_base = OTHER; note = N1-methylpseudouridine
  modified_base 2923: mod_base = OTHER; note = N1-methylpseudouridine
  modified_base 2929: mod_base = OTHER; note = N1-methylpseudouridine
  modified_base 2983: mod_base = OTHER; note = N1-methylpseudouridine
  modified_base 2992: mod_base = OTHER; note = N1-methylpseudouridine
  modified_base 3004: mod_base = OTHER; note = N1-methylpseudouridine
  modified_base 3013: mod_base = OTHER; note = N1-methylpseudouridine
  modified_base 3025: mod_base = OTHER; note = N1-methylpseudouridine
  modified_base 3028: mod_base = OTHER; note = N1-methylpseudouridine
  modified_base 3043: mod_base = OTHER; note = N1-methylpseudouridine
  modified_base 3058: mod_base = OTHER; note = N1-methylpseudouridine
  modified_base 3073: mod_base = OTHER; note = N1-methylpseudouridine
  modified_base 3075: mod_base = OTHER; note = N1-methylpseudouridine
  modified_base 3079: mod_base = OTHER; note = N1-methylpseudouridine
  modified_base 3081: mod_base = OTHER; note = N1-methylpseudouridine
  modified_base 3084: mod_base = OTHER; note = N1-methylpseudouridine
  modified_base 3088: mod_base = OTHER; note = N1-methylpseudouridine
  modified_base 3106: mod_base = OTHER; note = N1-methylpseudouridine
  modified_base 3108: mod_base = OTHER; note = N1-methylpseudouridine
  modified_base 3112: mod_base = OTHER; note = N1-methylpseudouridine
  modified_base 3124: mod_base = OTHER; note = N1-methylpseudouridine
  modified_base 3130: mod_base = OTHER; note = N1-methylpseudouridine
  modified_base 3139: mod_base = OTHER; note = N1-methylpseudouridine
  modified_base 3147: mod_base = OTHER; note = N1-methylpseudouridine
  modified_base 3154: mod_base = OTHER; note = N1-methylpseudouridine
  modified_base 3163: mod_base = OTHER; note = N1-methylpseudouridine
  modified_base 3166: mod_base = OTHER; note = N1-methylpseudouridine
  modified_base 3177: mod_base = OTHER; note = N1-methylpseudouridine
  modified_base 3178: mod_base = OTHER; note = N1-methylpseudouridine
  modified_base 3181: mod_base = OTHER; note = N1-methylpseudouridine
  modified_base 3196: mod_base = OTHER; note = N1-methylpseudouridine
  modified_base 3208: mod_base = OTHER; note = N1-methylpseudouridine
  modified_base 3211: mod_base = OTHER; note = N1-methylpseudouridine
  modified_base 3250: mod_base = OTHER; note = N1-methylpseudouridine
  modified_base 3265: mod_base = OTHER; note = N1-methylpseudouridine
  modified_base 3268: mod_base = OTHER; note = N1-methylpseudouridine
  modified_base 3277: mod_base = OTHER; note = N1-methylpseudouridine
  modified_base 3285: mod_base = OTHER; note = N1-methylpseudouridine
  modified_base 3288: mod_base = OTHER; note = N1-methylpseudouridine
  modified_base 3298: mod_base = OTHER; note = N1-methylpseudouridine
  modified_base 3301: mod_base = OTHER; note = N1-methylpseudouridine
  modified_base 3313: mod_base = OTHER; note = N1-methylpseudouridine
  modified_base 3316: mod_base = OTHER; note = N1-methylpseudouridine
  modified_base 3330: mod_base = OTHER; note = N1-methylpseudouridine
  modified_base 3331: mod_base = OTHER; note = N1-methylpseudouridine
  modified_base 3340: mod_base = OTHER; note = N1-methylpseudouridine
  modified_base 3364: mod_base = OTHER; note = N1-methylpseudouridine
  modified_base 3373: mod_base = OTHER; note = N1-methylpseudouridine
  modified_base 3387: mod_base = OTHER; note = N1-methylpseudouridine
  modified_base 3388: mod_base = OTHER; note = N1-methylpseudouridine
  modified_base 3391: mod_base = OTHER; note = N1-methylpseudouridine
  modified_base 3403: mod_base = OTHER; note = N1-methylpseudouridine
  modified_base 3406: mod_base = OTHER; note = N1-methylpseudouridine
  modified_base 3421: mod_base = OTHER; note = N1-methylpseudouridine
  modified_base 3433: mod_base = OTHER; note = N1-methylpseudouridine
  modified_base 3442: mod_base = OTHER; note = N1-methylpseudouridine
  modified_base 3445: mod_base = OTHER; note = N1-methylpseudouridine
  modified_base 3457: mod_base = OTHER; note = N1-methylpseudouridine
  modified_base 3468: mod_base = OTHER; note = N1-methylpseudouridine
  modified_base 3487: mod_base = OTHER; note = N1-methylpseudouridine
  modified_base 3490: mod_base = OTHER; note = N1-methylpseudouridine
  modified_base 3499: mod_base = OTHER; note = N1-methylpseudouridine
  modified_base 3505: mod_base = OTHER; note = N1-methylpseudouridine
  modified_base 3508: mod_base = OTHER; note = N1-methylpseudouridine
  modified_base 3514: mod_base = OTHER; note = N1-methylpseudouridine
  modified_base 3526: mod_base = OTHER; note = N1-methylpseudouridine
  modified_base 3529: mod_base = OTHER; note = N1-methylpseudouridine
  modified_base 3537: mod_base = OTHER; note = N1-methylpseudouridine
  modified_base 3538: mod_base = OTHER; note = N1-methylpseudouridine
  modified_base 3549: mod_base = OTHER; note = N1-methylpseudouridine
  modified_base 3550: mod_base = OTHER; note = N1-methylpseudouridine
  modified_base 3555: mod_base = OTHER; note = N1-methylpseudouridine
  modified_base 3556: mod_base = OTHER; note = N1-methylpseudouridine
  modified_base 3558: mod_base = OTHER; note = N1-methylpseudouridine
  modified_base 3565: mod_base = OTHER; note = N1-methylpseudouridine
  modified_base 3574: mod_base = OTHER; note = N1-methylpseudouridine
  modified_base 3582: mod_base = OTHER; note = N1-methylpseudouridine
  modified_base 3603: mod_base = OTHER; note = N1-methylpseudouridine
  modified_base 3607: mod_base = OTHER; note = N1-methylpseudouridine
  modified_base 3616: mod_base = OTHER; note = N1-methylpseudouridine
  modified_base 3619: mod_base = OTHER; note = N1-methylpseudouridine
  modified_base 3631: mod_base = OTHER; note = N1-methylpseudouridine
  modified_base 3634: mod_base = OTHER; note = N1-methylpseudouridine
  modified_base 3642: mod_base = OTHER; note = N1-methylpseudouridine
  modified_base 3652: mod_base = OTHER; note = N1-methylpseudouridine
  modified_base 3663: mod_base = OTHER; note = N1-methylpseudouridine
  modified_base 3664: mod_base = OTHER; note = N1-methylpseudouridine
  modified_base 3667: mod_base = OTHER; note = N1-methylpseudouridine
  modified_base 3669: mod_base = OTHER; note = N1-methylpseudouridine
  modified_base 3678: mod_base = OTHER; note = N1-methylpseudouridine
  modified_base 3685: mod_base = OTHER; note = N1-methylpseudouridine
  modified_base 3687: mod_base = OTHER; note = N1-methylpseudouridine
  modified_base 3694: mod_base = OTHER; note = N1-methylpseudouridine
  modified_base 3703: mod_base = OTHER; note = N1-methylpseudouridine
  modified_base 3706: mod_base = OTHER; note = N1-methylpseudouridine
  modified_base 3727: mod_base = OTHER; note = N1-methylpseudouridine
  modified_base 3747: mod_base = OTHER; note = N1-methylpseudouridine
  modified_base 3750: mod_base = OTHER; note = N1-methylpseudouridine
  modified_base 3751: mod_base = OTHER; note = N1-methylpseudouridine
  modified_base 3753: mod_base = OTHER; note = N1-methylpseudouridine
  modified_base 3754: mod_base = OTHER; note = N1-methylpseudouridine
  modified_base 3756: mod_base = OTHER; note = N1-methylpseudouridine
  modified_base 3766: mod_base = OTHER; note = N1-methylpseudouridine
  modified_base 3769: mod_base = OTHER; note = N1-methylpseudouridine
  modified_base 3774: mod_base = OTHER; note = N1-methylpseudouridine
  modified_base 3775: mod_base = OTHER; note = N1-methylpseudouridine
  modified_base 3777: mod_base = OTHER; note = N1-methylpseudouridine
  modified_base 3778: mod_base = OTHER; note = N1-methylpseudouridine
  modified_base 3790: mod_base = OTHER; note = N1-methylpseudouridine
  modified_base 3796: mod_base = OTHER; note = N1-methylpseudouridine
  modified_base 3811: mod_base = OTHER; note = N1-methylpseudouridine
  modified_base 3826: mod_base = OTHER; note = N1-methylpseudouridine
  modified_base 3829: mod_base = OTHER; note = N1-methylpseudouridine
  modified_base 3856: mod_base = OTHER; note = N1-methylpseudouridine
  modified_base 3859: mod_base = OTHER; note = N1-methylpseudouridine
  modified_base 3861: mod_base = OTHER; note = N1-methylpseudouridine
  modified_base 3879: mod_base = OTHER; note = N1-methylpseudouridine
  modified_base 3880: mod_base = OTHER; note = N1-methylpseudouridine
  modified_base 3889: mod_base = OTHER; note = N1-methylpseudouridine
  modified_base 3898: mod_base = OTHER; note = N1-methylpseudouridine
  modified_base 3901: mod_base = OTHER; note = N1-methylpseudouridine
  modified_base 3907: mod_base = OTHER; note = N1-methylpseudouridine
  modified_base 3916: mod_base = OTHER; note = N1-methylpseudouridine
  modified_base 3922: mod_base = OTHER; note = N1-methylpseudouridine
  modified_base 3925: mod_base = OTHER; note = N1-methylpseudouridine
  modified_base 3940: mod_base = OTHER; note = N1-methylpseudouridine
  modified_base 3954: mod_base = OTHER; note = N1-methylpseudouridine
  modified_base 3955: mod_base = OTHER; note = N1-methylpseudouridine
  modified_base 3970: mod_base = OTHER; note = N1-methylpseudouridine
  modified_base 3973: mod_base = OTHER; note = N1-methylpseudouridine
  modified_base 3997: mod_base = OTHER; note = N1-methylpseudouridine
  modified_base 4000: mod_base = OTHER; note = N1-methylpseudouridine
  modified_base 4017: mod_base = OTHER; note = N1-methylpseudouridine
  modified_base 4032: mod_base = OTHER; note = N1-methylpseudouridine
  modified_base 4041: mod_base = OTHER; note = N1-methylpseudouridine
  modified_base 4042: mod_base = OTHER; note = N1-methylpseudouridine
  modified_base 4057: mod_base = OTHER; note = N1-methylpseudouridine
  modified_base 4062: mod_base = OTHER; note = N1-methylpseudouridine
  modified_base 4069: mod_base = OTHER; note = N1-methylpseudouridine
  modified_base 4072: mod_base = OTHER; note = N1-methylpseudouridine
  modified_base 4075: mod_base = OTHER; note = N1-methylpseudouridine
  modified_base 4078: mod_base = OTHER; note = N1-methylpseudouridine
  modified_base 4087: mod_base = OTHER; note = N1-methylpseudouridine
  modified_base 4111: mod_base = OTHER; note = N1-methylpseudouridine
  modified_base 4120: mod_base = OTHER; note = N1-methylpseudouridine
  modified_base 4129: mod_base = OTHER; note = N1-methylpseudouridine
  modified_base 4132: mod_base = OTHER; note = N1-methylpseudouridine
  modified_base 4138: mod_base = OTHER; note = N1-methylpseudouridine
  modified_base 4144: mod_base = OTHER; note = N1-methylpseudouridine
  modified_base 4147: mod_base = OTHER; note = N1-methylpseudouridine
  modified_base 4161: mod_base = OTHER; note = N1-methylpseudouridine
  modified_base 4162: mod_base = OTHER; note = N1-methylpseudouridine
  modified_base 4177: mod_base = OTHER; note = N1-methylpseudouridine
  modified_base 4182: mod_base = OTHER; note = N1-methylpseudouridine
  modified_base 4183: mod_base = OTHER; note = N1-methylpseudouridine
  modified_base 4186: mod_base = OTHER; note = N1-methylpseudouridine
  modified_base 4200: mod_base = OTHER; note = N1-methylpseudouridine
  modified_base 4210: mod_base = OTHER; note = N1-methylpseudouridine
  modified_base 4222: mod_base = OTHER; note = N1-methylpseudouridine
  modified_base 4225: mod_base = OTHER; note = N1-methylpseudouridine
  modified_base 4228: mod_base = OTHER; note = N1-methylpseudouridine
  modified_base 4234: mod_base = OTHER; note = N1-methylpseudouridine
  modified_base 4242: mod_base = OTHER; note = N1-methylpseudouridine
  modified_base 4249: mod_base = OTHER; note = N1-methylpseudouridine
  modified_base 4251: mod_base = OTHER; note = N1-methylpseudouridine
  modified_base 4252: mod_base = OTHER; note = N1-methylpseudouridine
  modified_base 4258: mod_base = OTHER; note = N1-methylpseudouridine
  modified_base 4279: mod_base = OTHER; note = N1-methylpseudouridine
  modified_base 4282: mod_base = OTHER; note = N1-methylpseudouridine
  modified_base 4300: mod_base = OTHER; note = N1-methylpseudouridine
  modified_base 4318: mod_base = OTHER; note = N1-methylpseudouridine
  modified_base 4324: mod_base = OTHER; note = N1-methylpseudouridine
  modified_base 4335: mod_base = OTHER; note = N1-methylpseudouridine
  modified_base 4339: mod_base = OTHER; note = N1-methylpseudouridine
  modified_base 4344: mod_base = OTHER; note = N1-methylpseudouridine
  modified_base 4345: mod_base = OTHER; note = N1-methylpseudouridine
  modified_base 4348: mod_base = OTHER; note = N1-methylpseudouridine
  modified_base 4350: mod_base = OTHER; note = N1-methylpseudouridine
  modified_base 4354: mod_base = OTHER; note = N1-methylpseudouridine
  modified_base 4365: mod_base = OTHER; note = N1-methylpseudouridine
  modified_base 4375: mod_base = OTHER; note = N1-methylpseudouridine
  modified_base 4411: mod_base = OTHER; note = N1-methylpseudouridine
  modified_base 4413: mod_base = OTHER; note = N1-methylpseudouridine
  modified_base 4414: mod_base = OTHER; note = N1-methylpseudouridine
  modified_base 4417: mod_base = OTHER; note = N1-methylpseudouridine
  modified_base 4434: mod_base = OTHER; note = N1-methylpseudouridine
  modified_base 4438: mod_base = OTHER; note = N1-methylpseudouridine
  modified_base 4447: mod_base = OTHER; note = N1-methylpseudouridine
  modified_base 4450: mod_base = OTHER; note = N1-methylpseudouridine
  modified_base 4459: mod_base = OTHER; note = N1-methylpseudouridine
  modified_base 4467: mod_base = OTHER; note = N1-methylpseudouridine
  modified_base 4468: mod_base = OTHER; note = N1-methylpseudouridine
  modified_base 4480: mod_base = OTHER; note = N1-methylpseudouridine
  modified_base 4483: mod_base = OTHER; note = N1-methylpseudouridine
  modified_base 4486: mod_base = OTHER; note = N1-methylpseudouridine
  modified_base 4501: mod_base = OTHER; note = N1-methylpseudouridine
  modified_base 4510: mod_base = OTHER; note = N1-methylpseudouridine
  modified_base 4513: mod_base = OTHER; note = N1-methylpseudouridine
  modified_base 4521: mod_base = OTHER; note = N1-methylpseudouridine
  modified_base 4546: mod_base = OTHER; note = N1-methylpseudouridine
  modified_base 4567: mod_base = OTHER; note = N1-methylpseudouridine
  modified_base 4570: mod_base = OTHER; note = N1-methylpseudouridine
  modified_base 4576: mod_base = OTHER; note = N1-methylpseudouridine
  modified_base 4578: mod_base = OTHER; note = N1-methylpseudouridine
  modified_base 4579: mod_base = OTHER; note = N1-methylpseudouridine
  modified_base 4585: mod_base = OTHER; note = N1-methylpseudouridine
  modified_base 4594: mod_base = OTHER; note = N1-methylpseudouridine
  modified_base 4611: mod_base = OTHER; note = N1-methylpseudouridine
  modified_base 4612: mod_base = OTHER; note = N1-methylpseudouridine
  modified_base 4617: mod_base = OTHER; note = N1-methylpseudouridine
  modified_base 4620: mod_base = OTHER; note = N1-methylpseudouridine
  modified_base 4621: mod_base = OTHER; note = N1-methylpseudouridine
  modified_base 4633: mod_base = OTHER; note = N1-methylpseudouridine
  modified_base 4647: mod_base = OTHER; note = N1-methylpseudouridine
  modified_base 4666: mod_base = OTHER; note = N1-methylpseudouridine
  modified_base 4669: mod_base = OTHER; note = N1-methylpseudouridine
  modified_base 4681: mod_base = OTHER; note = N1-methylpseudouridine
  modified_base 4684: mod_base = OTHER; note = N1-methylpseudouridine
  modified_base 4696: mod_base = OTHER; note = N1-methylpseudouridine
  modified_base 4705: mod_base = OTHER; note = N1-methylpseudouridine
  modified_base 4707: mod_base = OTHER; note = N1-methylpseudouridine
  modified_base 4720: mod_base = OTHER; note = N1-methylpseudouridine
  modified_base 4726: mod_base = OTHER; note = N1-methylpseudouridine
  modified_base 4735: mod_base = OTHER; note = N1-methylpseudouridine
  modified_base 4782: mod_base = OTHER; note = N1-methylpseudouridine
  modified_base 4783: mod_base = OTHER; note = N1-methylpseudouridine
  modified_base 4810: mod_base = OTHER; note = N1-methylpseudouridine
  modified_base 4812: mod_base = OTHER; note = N1-methylpseudouridine
  modified_base 4823: mod_base = OTHER; note = N1-methylpseudouridine
  modified_base 4824: mod_base = OTHER; note = N1-methylpseudouridine
  modified_base 4827: mod_base = OTHER; note = N1-methylpseudouridine
  modified_base 4828: mod_base = OTHER; note = N1-methylpseudouridine
  modified_base 4833: mod_base = OTHER; note = N1-methylpseudouridine
  modified_base 4837: mod_base = OTHER; note = N1-methylpseudouridine
  modified_base 4838: mod_base = OTHER; note = N1-methylpseudouridine
  modified_base 4840: mod_base = OTHER; note = N1-methylpseudouridine
  modified_base 4848: mod_base = OTHER; note = N1-methylpseudouridine
  modified_base 4849: mod_base = OTHER; note = N1-methylpseudouridine
  modified_base 4853: mod_base = OTHER; note = N1-methylpseudouridine
  modified_base 4854: mod_base = OTHER; note = N1-methylpseudouridine
  modified_base 4856: mod_base = OTHER; note = N1-methylpseudouridine
  modified_base 4862: mod_base = OTHER; note = N1-methylpseudouridine
  modified_base 4867: mod_base = OTHER; note = N1-methylpseudouridine
  modified_base 4868: mod_base = OTHER; note = N1-methylpseudouridine
  modified_base 4870: mod_base = OTHER; note = N1-methylpseudouridine
  modified_base 4871: mod_base = OTHER; note = N1-methylpseudouridine
  modified_base 4873: mod_base = OTHER; note = N1-methylpseudouridine
  modified_base 4875: mod_base = OTHER; note = N1-methylpseudouridine
  modified_base 4879: mod_base = OTHER; note = N1-methylpseudouridine
  modified_base 4880: mod_base = OTHER; note = N1-methylpseudouridine
  modified_base 4886: mod_base = OTHER; note = N1-methylpseudouridine
  modified_base 4888: mod_base = OTHER; note = N1-methylpseudouridine
  modified_base 4892: mod_base = OTHER; note = N1-methylpseudouridine
  modified_base 4894: mod_base = OTHER; note = N1-methylpseudouridine
  modified_base 4895: mod_base = OTHER; note = N1-methylpseudouridine
  modified_base 4898: mod_base = OTHER; note = N1-methylpseudouridine
  modified_base 4900: mod_base = OTHER; note = N1-methylpseudouridine
  modified_base 4901: mod_base = OTHER; note = N1-methylpseudouridine
  modified_base 4902: mod_base = OTHER; note = N1-methylpseudouridine
  modified_base 4906: mod_base = OTHER; note = N1-methylpseudouridine
  modified_base 4913: mod_base = OTHER; note = N1-methylpseudouridine
  modified_base 4917: mod_base = OTHER; note = N1-methylpseudouridine
  modified_base 4924: mod_base = OTHER; note = N1-methylpseudouridine
  modified_base 4926: mod_base = OTHER; note = N1-methylpseudouridine
aggaaataagagagaaaagaagagtaagaagaaatataagagccaccatgagcgaggtggagttcagccacgagtactggatgcggcacgccctgaccctggccaagcgggcccgggacgagcgggaggtgcccgtgggcgccgtgctggtgctgaacaaccgggtgatcggcgagggctggaaccgggccatcggcctgcacgaccccaccgcccacgccgagatcatggccctgcggcagggcggcctggtgatgcagaactaccggctgatcgacgccaccctgtacgtgaccttcgagccctgcgtgatgtgcgccggcgccatgatccacagccggatcggccgggtggtgttcggcgtgcggaacgccaagaccggcgccgccggcagcctgatggacgtgctgcaccaccccggcatgaaccaccgggtggagatcaccgagggcatcctggccgacgagtgcgccgccctgctgtgccggttcttccggatgccccggcgggtgttcaacgcccagaagaaggcccagagcagcaccgacagcggcggcagcagcggcggcagcagcggcagcgagacacccggcaccagcgagagcgccacccccgagagcagcggcggcagcagcggcggcagcgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagacagccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccgcaagagcgaggagacaatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctcaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacacggcagatcaccaagcacgtggcccagatcctggacagccggatgaacaccaagtacgacgagaacgacaagctgatccgggaggtgaaggtgatcaccctcaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagacaaacggcgagacaggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctcaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacacggatcgacctgagccagctgggcggcgacgagggcgccgacaagcggaccgccgacggcagcgagttcgagagccccaagaagaagcggaaggtgtgagcggccgcttaattaagctgccttctgcggggcttgccttctggccatgcccttcttctctcccttgcacctgtacctcttggtctttgaataaagcctgagtaggaagtctaga

SEQ ID NO: 4 - RNA, 4950 nt, synthetic construct
  source 1..4950: mol_type = other RNA
  modified_base 6: mod_base = OTHER; note = N1-methylpseudouridine
  modified_base 9: mod_base = OTHER; note = N1-methylpseudouridine
  modified_base 15: mod_base = OTHER; note = N1-methylpseudouridine
  modified_base 18: mod_base = OTHER; note = N1-methylpseudouridine
  modified_base 20: mod_base = OTHER; note = N1-methylpseudouridine
  modified_base 24: mod_base = OTHER; note = N1-methylpseudouridine
  modified_base 26: mod_base = OTHER; note = N1-methylpseudouridine
  modified_base 28: mod_base = OTHER; note = N1-methylpseudouridine
  modified_base 32: mod_base = OTHER; note = N1-methylpseudouridine
  modified_base 34: mod_base = OTHER; note = N1-methylpseudouridine
  modified_base 36: mod_base = OTHER; note = N1-methylpseudouridine
  modified_base 40: mod_base = OTHER; note = N1-methylpseudouridine
  modified_base 42: mod_base = OTHER; note = N1-methylpseudouridine
  modified_base 48: mod_base = OTHER; note = N1-methylpseudouridine
  modified_base 53: mod_base = OTHER; note = N1-methylpseudouridine
  modified_base 55: mod_base = OTHER; note = N1-methylpseudouridine
  modified_base 66: mod_base = OTHER; note = N1-methylpseudouridine
  modified_base 75: mod_base = OTHER; note = N1-methylpseudouridine
  modified_base 80: mod_base = OTHER; note = N1-methylpseudouridine
  modified_base 81: mod_base = OTHER; note = N1-methylpseudouridine
  modified_base 92: mod_base = OTHER; note = N1-methylpseudouridine
  modified_base 95: mod_base = OTHER; note = N1-methylpseudouridine
  modified_base 99: mod_base = OTHER; note = N1-methylpseudouridine
  modified_base 111: mod_base = OTHER; note = N1-methylpseudouridine
  modified_base 117: mod_base = OTHER; note = N1-methylpseudouridine
  modified_base 147: mod_base = OTHER; note = N1-methylpseudouridine
  modified_base 153: mod_base = OTHER; note = N1-methylpseudouridine
  modified_base 162: mod_base = OTHER; note = N1-methylpseudouridine
  modified_base 165: mod_base = OTHER; note = N1-methylpseudouridine
  modified_base 168: mod_base = OTHER; note = N1-methylpseudouridine
  modified_base 171: mod_base = OTHER; note = N1-methylpseudouridine
  modified_base 183: mod_base = OTHER; note = N1-methylpseudouridine
  modified_base 186: mod_base = OTHER; note = N1-methylpseudouridine
  modified_base 197: mod_base = OTHER; note = N1-methylpseudouridine
  modified_base 210: mod_base = OTHER; note = N1-methylpseudouridine
  modified_base 216: mod_base = OTHER; note = N1-methylpseudouridine
  modified_base 243: mod_base = OTHER; note = N1-methylpseudouridine
  modified_base 246: mod_base = OTHER; note = N1-methylpseudouridine
  modified_base 252: mod_base = OTHER; note = N1-methylpseudouridine
  modified_base 267: mod_base = OTHER; note = N1-methylpseudouridine
  modified_base 270: mod_base = OTHER; note = N1-methylpseudouridine
  modified_base 273: mod_base = OTHER; note = N1-methylpseudouridine
  modified_base 281: mod_base = OTHER; note = N1-methylpseudouridine
  modified_base 288: mod_base = OTHER; note = N1-methylpseudouridine
  modified_base 291: mod_base = OTHER; note = N1-methylpseudouridine
  modified_base 303: mod_base = OTHER; note = N1-methylpseudouridine
  modified_base 305: mod_base = OTHER; note = N1-methylpseudouridine
  modified_base 309: mod_base = OTHER; note = N1-methylpseudouridine
  modified_base 314: mod_base = OTHER; note = N1-methylpseudouridine
  modified_base 315: mod_base = OTHER; note = N1-methylpseudouridine
  modified_base 323: mod_base = OTHER; note = N1-methylpseudouridine
  modified_base 327: mod_base = OTHER; note = N1-methylpseudouridine
  modified_base 330: mod_base = OTHER; note = N1-methylpseudouridine
  modified_base 332: mod_base = OTHER; note = N1-methylpseudouridine
  modified_base 345: mod_base = OTHER; note = N1-methylpseudouridine
  modified_base 348: mod_base = OTHER; note = N1-methylpseudouridine
  modified_base 360: mod_base = OTHER; note = N1-methylpseudouridine
  modified_base 369: mod_base = OTHER; note = N1-methylpseudouridine
  modified_base 372: mod_base = OTHER; note = N1-methylpseudouridine
  modified_base 374: mod_base = OTHER; note = N1-methylpseudouridine
  modified_base 375: mod_base = OTHER; note = N1-methylpseudouridine
  modified_base 381: mod_base = OTHER; note = N1-methylpseudouridine
  modified_base 414: mod_base = OTHER; note = N1-methylpseudouridine
  modified_base 417: mod_base = OTHER; note = N1-methylpseudouridine
  modified_base 423: mod_base = OTHER; note = N1-methylpseudouridine
  modified_base 426: mod_base = OTHER; note = N1-methylpseudouridine
  modified_base 441: mod_base = OTHER; note = N1-methylpseudouridine
  modified_base 453: mod_base = OTHER; note = N1-methylpseudouridine
  modified_base 459: mod_base = OTHER; note = N1-methylpseudouridine
  modified_base 471: mod_base = OTHER; note = N1-methylpseudouridine
  modified_base 474: mod_base = OTHER; note = N1-methylpseudouridine
  modified_base 485: mod_base = OTHER; note = N1-methylpseudouridine
  modified_base 495: mod_base = OTHER; note = N1-methylpseudouridine
  modified_base 498: mod_base = OTHER; note = N1-methylpseudouridine
  modified_base 500: mod_base = OTHER; note = N1-methylpseudouridine
  modified_base 506: mod_base = OTHER; note = N1-methylpseudouridine
  modified_base 507: mod_base = OTHER; note = N1-methylpseudouridine
  modified_base 509: mod_base = OTHER; note = N1-methylpseudouridine
  modified_base 510: mod_base = OTHER; note = N1-methylpseudouridine
  modified_base 516: mod_base = OTHER; note = N1-methylpseudouridine
  modified_base 528: mod_base = OTHER; note = N1-methylpseudouridine
  modified_base 530: mod_base = OTHER; note = N1-methylpseudouridine
  modified_base 531: mod_base = OTHER; note = N1-methylpseudouridine
  modified_base 671: mod_base = OTHER; note = N1-methylpseudouridine
  modified_base 678: mod_base = OTHER; note = N1-methylpseudouridine
  modified_base 684: mod_base = OTHER; note = N1-methylpseudouridine
  modified_base 690: mod_base = OTHER; note = N1-methylpseudouridine
  modified_base 705: mod_base = OTHER; note = N1-methylpseudouridine
  modified_base 710: mod_base = OTHER; note = N1-methylpseudouridine
  modified_base 717: mod_base = OTHER; note = N1-methylpseudouridine
  modified_base 720: mod_base = OTHER; note = N1-methylpseudouridine
  modified_base 731: mod_base = OTHER; note = N1-methylpseudouridine
  modified_base 738: mod_base = OTHER; note = N1-methylpseudouridine
  modified_base 752: mod_base = OTHER; note = N1-methylpseudouridine
  modified_base 753: mod_base = OTHER; note = N1-methylpseudouridine
  modified_base 759: mod_base = OTHER; note = N1-methylpseudouridine
  modified_base 762: mod_base = OTHER; note = N1-methylpseudouridine
  modified_base 786: mod_base = OTHER; note = N1-methylpseudouridine
  modified_base 798: mod_base = OTHER; note = N1-methylpseudouridine
  modified_base 801: mod_base = OTHER; note = N1-methylpseudouridine
  modified_base 810: mod_base = OTHER; note = N1-methylpseudouridine
  modified_base 813: mod_base = OTHER; note = N1-methylpseudouridine
  modified_base 815: mod_base = OTHER; note = N1-methylpseudouridine
  modified_base 816: mod_base = OTHER; note = N1-methylpseudouridine
  modified_base 849: mod_base = OTHER; note = N1-methylpseudouridine
  modified_base 872: mod_base = OTHER; note = N1-methylpseudouridine
  modified_base 894: mod_base = OTHER; note = N1-methylpseudouridine
  modified_base 896: mod_base = OTHER; note = N1-methylpseudouridine
  modified_base 899: mod_base = OTHER; note = N1-methylpseudouridine
  modified_base 903: mod_base = OTHER; note = N1-methylpseudouridine
  modified_base 912: mod_base = OTHER; note = N1-methylpseudouridine
  modified_base 914: mod_base = OTHER; note = N1-methylpseudouridine
  modified_base 915: mod_base = OTHER; note = N1-methylpseudouridine
  modified_base 927: mod_base = OTHER; note = N1-methylpseudouridine
  modified_base 936: mod_base = OTHER; note = N1-methylpseudouridine
  modified_base 947: mod_base = OTHER; note = N1-methylpseudouridine
  modified_base 948: mod_base = OTHER; note = N1-methylpseudouridine
  modified_base 950: mod_base = OTHER; note = N1-methylpseudouridine
  modified_base 951: mod_base = OTHER; note = N1-methylpseudouridine
  modified_base 960: mod_base = OTHER; note = N1-methylpseudouridine
  modified_base 971: mod_base = OTHER; note = N1-methylpseudouridine
  modified_base 972: mod_base = OTHER; note = N1-methylpseudouridine
  modified_base 975: mod_base = OTHER; note = N1-methylpseudouridine
  modified_base 978: mod_base = OTHER; note = N1-methylpseudouridine
  modified_base 1011: mod_base = OTHER; note = N1-methylpseudouridine
  modified_base 1013: mod_base = OTHER; note = N1-methylpseudouridine
  modified_base 1014: mod_base = OTHER; note = N1-methylpseudouridine
  modified_base 1023: mod_base = OTHER; note = N1-methylpseudouridine
  modified_base 1026: mod_base = OTHER; note = N1-methylpseudouridine
  modified_base 1035: mod_base = OTHER; note = N1-methylpseudouridine
  modified_base 1040: mod_base = OTHER; note = N1-methylpseudouridine
  modified_base 1052: mod_base = OTHER; note = N1-methylpseudouridine
  modified_base 1062: mod_base = OTHER; note = N1-methylpseudouridine
  modified_base 1064: mod_base = OTHER; note = N1-methylpseudouridine
  modified_base 1071: mod_base = OTHER; note = N1-methylpseudouridine
  modified_base 1083: mod_base = OTHER; note = N1-methylpseudouridine
  modified_base 1086: mod_base = OTHER; note = N1-methylpseudouridine
  modified_base 1110: mod_base = OTHER; note = N1-methylpseudouridine
  modified_base 1116: mod_base = OTHER; note = N1-methylpseudouridine
  modified_base 1119: mod_base = OTHER; note = N1-methylpseudouridine
  modified_base 1121: mod_base = OTHER; note = N1-methylpseudouridine
  modified_base 1125: mod_base = OTHER; note = N1-methylpseudouridine
  modified_base 1131: mod_base = OTHER; note = N1-methylpseudouridine
  modified_base 1140: mod_base = OTHER; note = N1-methylpseudouridine
  modified_base 1143: mod_base = OTHER; note = N1-methylpseudouridine
  modified_base 1148: mod_base = OTHER; note = N1-methylpseudouridine
  modified_base 1149: mod_base = OTHER; note = N1-methylpseudouridine
  modified_base 1160: mod_base = OTHER; note = N1-methylpseudouridine
  modified_base 1161: mod_base = OTHER; note = N1-methylpseudouridine
  modified_base 1164: mod_base = OTHER; note = N1-methylpseudouridine
  modified_base 1167: mod_base = OTHER; note = N1-methylpseudouridine
  modified_base 1179: mod_base = OTHER; note = N1-methylpseudouridine
  modified_base 1200: mod_base = OTHER; note = N1-methylpseudouridine
  modified_base 1209: mod_base = OTHER; note = N1-methylpseudouridine
  modified_base 1211: mod_base = OTHER; note = N1-methylpseudouridine
  modified_base 1212: mod_base = OTHER; note = N1-methylpseudouridine
  modified_base 1215: mod_base = OTHER; note = N1-methylpseudouridine
  modified_base 1221: mod_base = OTHER; note = N1-methylpseudouridine
  modified_base 1224: mod_base = OTHER; note = N1-methylpseudouridine
  modified_base 1232: mod_base = OTHER; note = N1-methylpseudouridine
  modified_base 1242: mod_base = OTHER; note = N1-methylpseudouridine
  modified_base 1244: mod_base = OTHER; note = N1-methylpseudouridine
  modified_base 1245: mod_base = OTHER; note = N1-methylpseudouridine
  modified_base 1260: mod_base = OTHER; note = N1-methylpseudouridine
  modified_base 1275: mod_base = OTHER; note = N1-methylpseudouridine
  modified_base 1290: mod_base = OTHER; note = N1-methylpseudouridine
  modified_base 1293: mod_base = OTHER; note = N1-methylpseudouridine
  modified_base 1305: mod_base = OTHER; note = N1-methylpseudouridine
  modified_base 1323: mod_base = OTHER; note = N1-methylpseudouridine
  modified_base 1332: mod_base = OTHER; note = N1-methylpseudouridine
  modified_base 1335: mod_base = OTHER; note = N1-methylpseudouridine
  modified_base 1344: mod_base = OTHER; note = N1-methylpseudouridine
  modified_base 1368: mod_base = OTHER; note = N1-methylpseudouridine
  modified_base 1370: mod_base = OTHER; note = N1-methylpseudouridine
  modified_base 1371: mod_base = OTHER; note = N1-methylpseudouridine
  modified_base 1380: mod_base = OTHER; note = N1-methylpseudouridine
  modified_base 1383: mod_base = OTHER; note = N1-methylpseudouridine
  modified_base 1389: mod_base = OTHER; note = N1-methylpseudouridine
  modified_base 1395: mod_base = OTHER; note = N1-methylpseudouridine
  modified_base 1401: mod_base = OTHER; note = N1-methylpseudouridine
  modified_base 1412: mod_base = OTHER; note = N1-methylpseudouridine
  modified_base 1413: mod_base = OTHER; note = N1-methylpseudouridine
  modified_base 1424: mod_base = OTHER; note = N1-methylpseudouridine
  modified_base 1425: mod_base = OTHER; note = N1-methylpseudouridine
  modified_base 1431: mod_base = OTHER; note = N1-methylpseudouridine
  modified_base 1449: mod_base = OTHER; note = N1-methylpseudouridine
  modified_base 1455: mod_base = OTHER; note = N1-methylpseudouridine
  modified_base 1469: mod_base = OTHER; note = N1-methylpseudouridine
  modified_base 1482: mod_base = OTHER; note = N1-methylpseudouridine
  modified_base 1491: mod_base = OTHER; note = N1-methylpseudouridine
  modified_base 1494: mod_base = OTHER; note = N1-methylpseudouridine
  modified_base 1503: mod_base = OTHER; note = N1-methylpseudouridine
  modified_base 1514: mod_base = OTHER; note = N1-methylpseudouridine
  modified_base 1524: mod_base = OTHER; note = N1-methylpseudouridine
  modified_base 1526: mod_base = OTHER; note = N1-methylpseudouridine
  modified_base 1527: mod_base = OTHER; note = N1-methylpseudouridine
  modified_base 1530: mod_base = OTHER; note = N1-methylpseudouridine
  modified_base 1545: mod_base = OTHER; note = N1-methylpseudouridine
  modified_base 1557: mod_base = OTHER; note = N1-methylpseudouridine
  modified_base 1560: mod_base = OTHER; note = N1-methylpseudouridine
  modified_base 1563: mod_base = OTHER; note = N1-methylpseudouridine
  modified_base 1572: mod_base = OTHER; note = N1-methylpseudouridine
  modified_base 1575: mod_base = OTHER; note = N1-methylpseudouridine
  modified_base 1581: mod_base = OTHER; note = N1-methylpseudouridine
  modified_base 1593: mod_base = OTHER; note = N1-methylpseudouridine
  modified_base 1608: mod_base = OTHER; note = N1-methylpseudouridine
  modified_base 1620: mod_base = OTHER; note = N1-methylpseudouridine
  modified_base 1623: mod_base = OTHER; note = N1-methylpseudouridine
  modified_base 1631: mod_base = OTHER; note = N1-methylpseudouridine
  modified_base 1653: mod_base = OTHER; note = N1-methylpseudouridine
  modified_base 1659: mod_base = OTHER; note = N1-methylpseudouridine
  modified_base 1662: mod_base = OTHER; note = N1-methylpseudouridine
  modified_base 1671: mod_base = OTHER; note = N1-methylpseudouridine
  modified_base 1674: mod_base = OTHER; note = N1-methylpseudouridine
  modified_base 1686: mod_base = OTHER; note = N1-methylpseudouridine
  modified_base 1697: mod_base = OTHER; note = N1-methylpseudouridine
  modified_base 1707: mod_base = OTHER; note = N1-methylpseudouridine
  modified_base 1709: mod_base = OTHER; note = N1-methylpseudouridine
  modified_base 1710: mod_base = OTHER; note = N1-methylpseudouridine
  modified_base 1712: mod_base = OTHER; note = N1-methylpseudouridine
  modified_base 1713: mod_base = OTHER; note = N1-methylpseudouridine
  modified_base 1733: mod_base = OTHER; note = N1-methylpseudouridine
  modified_base 1742: mod_base = OTHER; note = N1-methylpseudouridine
  modified_base 1746: mod_base = OTHER; note = N1-methylpseudouridine
  modified_base 1772: mod_base = OTHER; note = N1-methylpseudouridine
  modified_base 1773: mod_base = OTHER; note = N1-methylpseudouridine
  modified_base 1775: mod_base = OTHER; note = N1-methylpseudouridine
  modified_base 1781: mod_base = OTHER; note = N1-methylpseudouridine
  modified_base 1782: mod_base = OTHER; note = N1-methylpseudouridine
  modified_base 1785: mod_base = OTHER; note = N1-methylpseudouridine
  modified_base 1794: mod_base = OTHER; note = N1-methylpseudouridine
  modified_base 1797: mod_base = OTHER; note = N1-methylpseudouridine
  modified_base 1806: mod_base = OTHER; note = N1-methylpseudouridine
  modified_base 1824: mod_base = OTHER; note = N1-methylpseudouridine
  modified_base 1827: mod_base = OTHER; note = N1-methylpseudouridine
  modified_base 1830: mod_base = OTHER; note = N1-methylpseudouridine
  modified_base 1836: mod_base = OTHER; note = N1-methylpseudouridine
  modified_base 1851: mod_base = OTHER; note = N1-methylpseudouridine
  modified_base 1854: mod_base = OTHER; note = N1-methylpseudouridine
  modified_base 1871: mod_base = OTHER; note = N1-methylpseudouridine
  modified_base 1872: mod_base = OTHER; note = N1-methylpseudouridine
  modified_base 1887: mod_base = OTHER; note = N1-methylpseudouridine
  modified_base 1899: mod_base = OTHER; note = N1-methylpseudouridine
  modified_base 1905: mod_base = OTHER; note = N1-methylpseudouridine
  modified_base 1914: mod_base = OTHER; note = N1-methylpseudouridine
  modified_base 1923: mod_base = OTHER; note = N1-methylpseudouridine
  modified_base 1926: mod_base = OTHER; note = N1-methylpseudouridine
  modified_base 1943: mod_base = OTHER; note = N1-methylpseudouridine
  modified_base 1944: mod_base = OTHER; note = N1-methylpseudouridine
  modified_base 1946: mod_base = OTHER; note = N1-methylpseudouridine
  modified_base 1952: mod_base = OTHER; note = N1-methylpseudouridine
  modified_base 1953: mod_base = OTHER; note = N1-methylpseudouridine
  modified_base 1956: mod_base = OTHER; note = N1-methylpseudouridine
  modified_base 1977: mod_base = OTHER; note = N1-methylpseudouridine
  modified_base 1986: mod_base = OTHER; note = N1-methylpseudouridine
  modified_base 1989: mod_base = OTHER; note = N1-methylpseudouridine
  modified_base 1994: mod_base = OTHER; note = N1-methylpseudouridine
  modified_base 1995: mod_base = OTHER; note = N1-methylpseudouridine
  modified_base 2001: mod_base = OTHER; note = N1-methylpseudouridine
  modified_base 2006: mod_base = OTHER; note = N1-methylpseudouridine
  modified_base 2009: mod_base = OTHER; note = N1-methylpseudouridine
  modified_base 2013: mod_base = OTHER; note = N1-methylpseudouridine
  modified_base 2022: mod_base = OTHER; note = N1-methylpseudouridine
  modified_base 2042: mod_base = OTHER; note = N1-methylpseudouridine
  modified_base 2043: mod_base = OTHER; note = N1-methylpseudouridine
  modified_base 2048: mod_base = OTHER; note = N1-methylpseudouridine
  modified_base 2052: mod_base = OTHER; note = N1-methylpseudouridine
  modified_base 2076: mod_base = OTHER; note = N1-methylpseudouridine
  modified_base 2084: mod_base = OTHER; note = N1-methylpseudouridine
  modified_base 2090: mod_base = OTHER; note = N1-methylpseudouridine
  modified_base 2091: mod_base = OTHER; note = N1-methylpseudouridine
  modified_base 2100: mod_base = OTHER; note = N1-methylpseudouridine
  modified_base 2103: mod_base = OTHER; note = N1-methylpseudouridine
  modified_base 2129: mod_base = OTHER; note = N1-methylpseudouridine
  modified_base 2130: mod_base = OTHER; note = N1-methylpseudouridine
  modified_base 2133: mod_base = OTHER; note = N1-methylpseudouridine
  modified_base 2142: mod_base = OTHER; note = N1-methylpseudouridine
  modified_base 2150: mod_base = OTHER; note = N1-methylpseudouridine
  modified_base 2151: mod_base = OTHER; note = N1-methylpseudouridine
  modified_base 2163: mod_base = OTHER; note = N1-methylpseudouridine
  modified_base 2178: mod_base = OTHER; note = N1-methylpseudouridine
  modified_base 2181: mod_base = OTHER; note = N1-methylpseudouridine
  modified_base 2196: mod_base = OTHER; note = N1-methylpseudouridine
  modified_base 2199: mod_base = OTHER; note = N1-methylpseudouridine
  modified_base 2201: mod_base = OTHER; note = N1-methylpseudouridine
  modified_base 2207: mod_base = OTHER; note = N1-methylpseudouridine
  modified_base 2210: mod_base = OTHER; note = N1-methylpseudouridine
  modified_base 2211: mod_base = OTHER; note = N1-methylpseudouridine
  modified_base 2217: mod_base = OTHER; note = N1-methylpseudouridine
  modified_base 2219: mod_base = OTHER; note = N1-methylpseudouridine
  modified_base 2229: mod_base = OTHER; note = N1-methylpseudouridine
  modified_base 2238: mod_base = OTHER; note = N1-methylpseudouridine
  modified_base 2243: mod_base = OTHER; note = N1-methylpseudouridine
  modified_base 2247: mod_base = OTHER; note = N1-methylpseudouridine
  modified_base 2259: mod_base = OTHER; note = N1-methylpseudouridine
  modified_base 2273: mod_base = OTHER; note = N1-methylpseudouridine
  modified_base 2274: mod_base = OTHER; note = N1-methylpseudouridine
  modified_base 2277: mod_base = OTHER; note = N1-methylpseudouridine
  modified_base 2301: mod_base = OTHER; note = N1-methylpseudouridine
  modified_base 2304: mod_base = OTHER; note = N1-methylpseudouridine
  modified_base 2310: mod_base = OTHER; note = N1-methylpseudouridine
  modified_base 2313: mod_base = OTHER; note = N1-methylpseudouridine
  modified_base 2315: mod_base = OTHER; note = N1-methylpseudouridine
  modified_base 2316: mod_base = OTHER; note = N1-methylpseudouridine
  modified_base 2334: mod_base = OTHER; note = N1-methylpseudouridine
  modified_base 2340: mod_base = OTHER; note = N1-methylpseudouridine
  modified_base 2349: mod_base = OTHER; note = N1-methylpseudouridine
  modified_base 2360: mod_base = OTHER; note = N1-methylpseudouridine
  modified_base 2363: mod_base = OTHER; note = N1-methylpseudouridine
  modified_base 2364: mod_base = OTHER; note = N1-methylpseudouridine
  modified_base 2373: mod_base = OTHER; note = N1-methylpseudouridine
  modified_base 2378: mod_base = OTHER; note = N1-methylpseudouridine
  modified_base 2381: mod_base = OTHER; note = N1-methylpseudouridine
  modified_base 2382: mod_base = OTHER; note = N1-methylpseudouridine
  modified_base 2391: mod_base = OTHER; note = N1-methylpseudouridine
  modified_base 2397: mod_base = OTHER; note = N1-methylpseudouridine
  modified_base 2406: mod_base = OTHER; note = N1-methylpseudouridine
  modified_base 2417: mod_base = OTHER; note = N1-methylpseudouridine
  modified_base 2418: mod_base = OTHER; note = N1-methylpseudouridine
  modified_base 2430: mod_base = OTHER; note = N1-methylpseudouridine
  modified_base 2438: mod_base = OTHER; note = N1-methylpseudouridine
  modified_base 2448: mod_base = OTHER; note = N1-methylpseudouridine
  modified_base 2451: mod_base = OTHER; note = N1-methylpseudouridine
  modified_base 2457: mod_base = OTHER; note = N1-methylpseudouridine
  modified_base 2460: mod_base = OTHER; note = N1-methylpseudouridine
  modified_base 2474: mod_base = OTHER; note = N1-methylpseudouridine
  modified_base 2475: mod_base = OTHER; note = N1-methylpseudouridine
  modified_base 2478: mod_base = OTHER; note = N1-methylpseudouridine
  modified_base 2502: mod_base = OTHER; note = N1-methylpseudouridine
  modified_base 2505: mod_base = OTHER; note = N1-methylpseudouridine
  modified_base 2514: mod_base = OTHER; note = N1-methylpseudouridine
  modified_base 2517: mod_base = OTHER; note = N1-methylpseudouridine
  modified_base 2520: mod_base = OTHER; note = N1-methylpseudouridine
  modified_base 2526: mod_base = OTHER; note = N1-methylpseudouridine
  modified_base 2532: mod_base = OTHER; note = N1-methylpseudouridine
  modified_base 2534: mod_base = OTHER; note = N1-methylpseudouridine
  modified_base 2535: mod_base = OTHER; note = N1-methylpseudouridine
  modified_base 2550: mod_base = OTHER; note = N1-methylpseudouridine
  modified_base 2553: mod_base = OTHER; note = N1-methylpseudouridine
  modified_base 2565: mod_base = OTHER; note = N1-methylpseudouridine
  modified_base 2573: mod_base = OTHER; note = N1-methylpseudouridine
  modified_base 2583: mod_base = OTHER; note = N1-methylpseudouridine
  modified_base 2585: mod_base = OTHER; note = N1-methylpseudouridine
  modified_base 2586: mod_base = OTHER; note = N1-methylpseudouridine
  modified_base 2598: mod_base = OTHER; note = N1-methylpseudouridine
  modified_base 2601: mod_base = OTHER; note = N1-methylpseudouridine
  modified_base 2610: mod_base = OTHER; note = N1-methylpseudouridine
  modified_base 2624: mod_base = OTHER; note = N1-methylpseudouridine
  modified_base 2633: mod_base = OTHER; note = N1-methylpseudouridine
  modified_base 2643: mod_base = OTHER; note = N1-methylpseudouridine
  modified_base 2655: mod_base = OTHER; note = N1-methylpseudouridine
  modified_base 2658: mod_base = OTHER; note = N1-methylpseudouridine
  modified_base 2667: mod_base = OTHER; note = N1-methylpseudouridine
  modified_base 2694: mod_base = OTHER; note = N1-methylpseudouridine
  modified_base 2697: mod_base = OTHER; note = N1-methylpseudouridine
  modified_base 2702: mod_base = OTHER; note = N1-methylpseudouridine
  modified_base 2703: mod_base = OTHER; note = N1-methylpseudouridine
  modified_base 2706: mod_base = OTHER; note = N1-methylpseudouridine
  modified_base 2720: mod_base = OTHER; note = N1-methylpseudouridine
  modified_base 2721: mod_base = OTHER; note = N1-methylpseudouridine
  modified_base 2735: mod_base = OTHER; note = N1-methylpseudouridine
  modified_base 2736: mod_base = OTHER; note = N1-methylpseudouridine
  modified_base 2739: mod_base = OTHER; note = N1-methylpseudouridine
  modified_base 2745: mod_base = OTHER; note = N1-methylpseudouridine
  modified_base 2748: mod_base = OTHER; note = N1-methylpseudouridine
  modified_base 2763: mod_base = OTHER; note = N1-methylpseudouridine
  modified_base 2768: mod_base = OTHER; note = N1-methylpseudouridine
  modified_base 2769: mod_base = OTHER; note = N1-methylpseudouridine
  modified_base 2781: mod_base = OTHER; note = N1-methylpseudouridine
  modified_base 2796: mod_base = OTHER; note = N1-methylpseudouridine
  modified_base 2817: mod_base = OTHER; note = N1-methylpseudouridine
  modified_base 2829: mod_base = OTHER; note = N1-methylpseudouridine
  modified_base 2838: mod_base = OTHER; note = N1-methylpseudouridine
  modified_base 2856: mod_base = OTHER; note = N1-methylpseudouridine
  modified_base 2868: mod_base = OTHER; note = N1-methylpseudouridine
  modified_base 2871: mod_base = OTHER; note = N1-methylpseudouridine
  modified_base 2880: mod_base = OTHER; note = N1-methylpseudouridine
  modified_base 2886: mod_base = OTHER; note = N1-methylpseudouridine
  modified_base 2889: mod_base = OTHER; note = N1-methylpseudouridine
  modified_base 2898: mod_base = OTHER; note = N1-methylpseudouridine
  modified_base 2901: mod_base = OTHER; note = N1-methylpseudouridine
  modified_base 2907: mod_base = OTHER; note = N1-methylpseudouridine
  modified_base 2910: mod_base = OTHER; note = N1-methylpseudouridine
  modified_base 2934: mod_base = OTHER; note = N1-methylpseudouridine
  modified_base 2937: mod_base = OTHER; note = N1-methylpseudouridine
  modified_base 2940: mod_base = OTHER; note = N1-methylpseudouridine
  modified_base 2946: mod_base = OTHER; note = N1-methylpseudouridine
  modified_base 3000: mod_base = OTHER; note = N1-methylpseudouridine
  modified_base 3009: mod_base = OTHER; note = N1-methylpseudouridine
  modified_base 3021: mod_base = OTHER; note = N1-methylpseudouridine
  modified_base 3030: mod_base = OTHER; note = N1-methylpseudouridine
  modified_base 3042: mod_base = OTHER; note = N1-methylpseudouridine
  modified_base 3045: mod_base = OTHER; note = N1-methylpseudouridine
  modified_base 3060: mod_base = OTHER; note = N1-methylpseudouridine
  modified_base 3075: mod_base = OTHER; note = N1-methylpseudouridine
  modified_base 3090: mod_base = OTHER; note = N1-methylpseudouridine
  modified_base 3092: mod_base = OTHER; note = N1-methylpseudouridine
  modified_base 3096: mod_base = OTHER; note = N1-methylpseudouridine
  modified_base 3098: mod_base = OTHER; note = N1-methylpseudouridine
  modified_base 3101: mod_base = OTHER; note = N1-methylpseudouridine
  modified_base 3105: mod_base = OTHER; note = N1-methylpseudouridine
  modified_base 3123: mod_base = OTHER; note = N1-methylpseudouridine
  modified_base 3125: mod_base = OTHER; note = N1-methylpseudouridine
  modified_base 3129: mod_base = OTHER; note = N1-methylpseudouridine
  modified_base 3141: mod_base = OTHER; note = N1-methylpseudouridine
  modified_base 3147: mod_base = OTHER; note = N1-methylpseudouridine
  modified_base 3156: mod_base = OTHER; note = N1-methylpseudouridine
  modified_base 3164: mod_base = OTHER; note = N1-methylpseudouridine
  modified_base 3171: mod_base = OTHER; note = N1-methylpseudouridine
  modified_base 3180: mod_base = OTHER; note = N1-methylpseudouridine
  modified_base 3183: mod_base = OTHER; note = N1-methylpseudouridine
  modified_base 3194: mod_base = OTHER; note = N1-methylpseudouridine
  modified_base 3195: mod_base = OTHER; note = N1-methylpseudouridine
  modified_base 3198: mod_base = OTHER; note = N1-methylpseudouridine
  modified_base 3213: mod_base = OTHER; note = N1-methylpseudouridine
  modified_base 3225: mod_base = OTHER; note = N1-methylpseudouridine
  modified_base 3228: mod_base = OTHER; note = N1-methylpseudouridine
  modified_base 3267: mod_base = OTHER; note = N1-methylpseudouridine
  modified_base 3282: mod_base = OTHER; note = N1-methylpseudouridine
  modified_base 3285: mod_base = OTHER; note = N1-methylpseudouridine
  modified_base 3294: mod_base = OTHER; note = N1-methylpseudouridine
  modified_base 3302: mod_base = OTHER; note = N1-methylpseudouridine
  modified_base 3305: mod_base = OTHER; note = N1-methylpseudouridine
  modified_base 3315: mod_base = OTHER; note = N1-methylpseudouridine
  modified_base 3318: mod_base = OTHER; note = N1-methylpseudouridine
  modified_base 3330: mod_base = OTHER; note = N1-methylpseudouridine
  modified_base 3333: mod_base = OTHER; note = N1-methylpseudouridine
  modified_base 3347: mod_base = OTHER; note = N1-methylpseudouridine
  modified_base 3348: mod_base = OTHER; note = N1-methylpseudouridine
  modified_base 3357: mod_base = OTHER; note = N1-methylpseudouridine
  modified_base 3381: mod_base = OTHER; note = N1-methylpseudouridine
  modified_base 3390: mod_base = OTHER; note = N1-methylpseudouridine
  modified_base 3404: mod_base = OTHER; note = N1-methylpseudouridine
  modified_base 3405: mod_base = OTHER; note = N1-methylpseudouridine
  modified_base 3408: mod_base = OTHER; note = N1-methylpseudouridine
  modified_base 3420: mod_base = OTHER; note = N1-methylpseudouridine
  modified_base 3423: mod_base = OTHER; note = N1-methylpseudouridine
  modified_base 3438: mod_base = OTHER; note = N1-methylpseudouridine
  modified_base 3450: mod_base = OTHER; note = N1-methylpseudouridine
  modified_base 3459: mod_base = OTHER; note = N1-methylpseudouridine
  modified_base 3462: mod_base = OTHER; note = N1-methylpseudouridine
  modified_base 3474: mod_base = OTHER; note = N1-methylpseudouridine
  modified_base 3485: mod_base = OTHER; note = N1-methylpseudouridine
  modified_base 3504: mod_base = OTHER; note = N1-methylpseudouridine
  modified_base 3507: mod_base = OTHER; note = N1-methylpseudouridine
  modified_base 3516: mod_base = OTHER; note = N1-methylpseudouridine
  modified_base 3522: mod_base = OTHER; note = N1-methylpseudouridine
  modified_base 3525: mod_base = OTHER; note = N1-methylpseudouridine
  modified_base 3531: mod_base = OTHER; note = N1-methylpseudouridine
  modified_base 3543: mod_base = OTHER; note = N1-methylpseudouridine
  modified_base 3546: mod_base = OTHER; note = N1-methylpseudouridine
  modified_base 3554: mod_base = OTHER; note = N1-methylpseudouridine
  modified_base 3555: mod_base = OTHER; note = N1-methylpseudouridine
  modified_base 3566: mod_base = OTHER; note = N1-methylpseudouridine
  modified_base 3567: mod_base = OTHER; note = N1-methylpseudouridine
  modified_base 3572: mod_base = OTHER; note = N1-methylpseudouridine
  modified_base 3573: mod_base = OTHER; note = N1-methylpseudouridine
  modified_base 3575: mod_base = OTHER; note = N1-methylpseudouridine
  modified_base 3582: mod_base = OTHER; note = N1-methylpseudouridine
  modified_base 3591: mod_base = OTHER; note = N1-methylpseudouridine
  modified_base 3599: mod_base = OTHER; note = N1-methylpseudouridine
  modified_base 3620: mod_base = OTHER; note = N1-methylpseudouridine
  modified_base 3624: mod_base = OTHER; note = N1-methylpseudouridine
  modified_base 3633: mod_base = OTHER; note = N1-methylpseudouridine
  modified_base 3636: mod_base = OTHER; note = N1-methylpseudouridine
  modified_base 3648: mod_base = OTHER; note = N1-methylpseudouridine
  modified_base 3651: mod_base = OTHER; note = N1-methylpseudouridine
  modified_base 3659: mod_base = OTHER; note = N1-methylpseudouridine
  modified_base 3669: mod_base = OTHER; note = N1-methylpseudouridine
  modified_base 3680: mod_base = OTHER; note = N1-methylpseudouridine
  modified_base 3681: mod_base = OTHER; note = N1-methylpseudouridine
  modified_base 3684: mod_base = OTHER; note = N1-methylpseudouridine
  modified_base 3686: mod_base = OTHER; note = N1-methylpseudouridine
  modified_base 3695: mod_base = OTHER; note = N1-methylpseudouridine
  modified_base 3702: mod_base = OTHER; note = N1-methylpseudouridine
  modified_base 3704: mod_base = OTHER; note = N1-methylpseudouridine
  modified_base 3711: mod_base = OTHER; note = N1-methylpseudouridine
  modified_base 3720: mod_base = OTHER; note = N1-methylpseudouridine
  modified_base 3723: mod_base = OTHER; note = N1-methylpseudouridine
  modified_base 3744: mod_base = OTHER; note = N1-methylpseudouridine
  modified_base 3764: mod_base = OTHER; note = N1-methylpseudouridine
  modified_base 3767: mod_base = OTHER; note = N1-methylpseudouridine
  modified_base 3768: mod_base = OTHER; note = N1-methylpseudouridine
  modified_base 3770: mod_base = OTHER; note = N1-methylpseudouridine
  modified_base 3771: mod_base = OTHER; note = N1-methylpseudouridine
  modified_base 3773: mod_base = OTHER; note = N1-methylpseudouridine
  modified_base 3783: mod_base = OTHER; note = N1-methylpseudouridine
  modified_base 3786: mod_base = OTHER; note = N1-methylpseudouridine
  modified_base 3791: mod_base = OTHER; note = N1-methylpseudouridine
  modified_base 3792: mod_base = OTHER; note = N1-methylpseudouridine
  modified_base 3794: mod_base = OTHER; note = N1-methylpseudouridine
  modified_base 3795: mod_base = OTHER; note = N1-methylpseudouridine
  modified_base 3807: mod_base = OTHER; note = N1-methylpseudouridine
  modified_base 3813: mod_base = OTHER; note = N1-methylpseudouridine
  modified_base 3828: mod_base = OTHER; note = N1-methylpseudouridine
  modified_base 3843: mod_base = OTHER; note = N1-methylpseudouridine
  modified_base 3846: mod_base = OTHER; note = N1-methylpseudouridine
  modified_base 3873: mod_base = OTHER; note = N1-methylpseudouridine
  modified_base 3876: mod_base = OTHER; note = N1-methylpseudouridine
  modified_base 3878: mod_base = OTHER; note = N1-methylpseudouridine
  modified_base 3896: mod_base = OTHER; note = N1-methylpseudouridine
  modified_base 3897: mod_base = OTHER; note = N1-methylpseudouridine
  modified_base 3906: mod_base = OTHER; note = N1-methylpseudouridine
  modified_base 3915: mod_base = OTHER; note = N1-methylpseudouridine
  modified_base 3918: mod_base = OTHER; note = N1-methylpseudouridine
  modified_base 3924: mod_base = OTHER; note = N1-methylpseudouridine
  modified_base 3933: mod_base = OTHER; note = N1-methylpseudouridine
  modified_base 3939: mod_base = OTHER; note = N1-methylpseudouridine
  modified_base 3942: mod_base = OTHER; note = N1-methylpseudouridine
  modified_base 3957: mod_base = OTHER; note = N1-methylpseudouridine
  modified_base 3971: mod_base = OTHER; note = N1-methylpseudouridine
  modified_base 3972: mod_base = OTHER; note = N1-methylpseudouridine
  modified_base 3987: mod_base = OTHER; note = N1-methylpseudouridine
  modified_base 3990: mod_base = OTHER; note = N1-methylpseudouridine
  modified_base 4014: mod_base = OTHER; note = N1-methylpseudouridine
  modified_base 4017: mod_base = OTHER; note = N1-methylpseudouridine
  modified_base 4034: mod_base = OTHER; note = N1-methylpseudouridine
  modified_base 4049: mod_base = OTHER; note = N1-methylpseudouridine
  modified_base 4058: mod_base = OTHER; note = N1-methylpseudouridine
  modified_base 4059: mod_base = OTHER; note = N1-methylpseudouridine
  modified_base 4074: mod_base = OTHER; note = N1-methylpseudouridine
  modified_base 4079: mod_base = OTHER; note = N1-methylpseudouridine
  modified_base 4086: mod_base = OTHER; note = N1-methylpseudouridine
  modified_base 4089: mod_base = OTHER; note = N1-methylpseudouridine
  modified_base 4092: mod_base = OTHER; note = N1-methylpseudouridine
  modified_base 4095: mod_base = OTHER; note = N1-methylpseudouridine
  modified_base 4104: mod_base = OTHER; note = N1-methylpseudouridine
  modified_base 4128: mod_base = OTHER; note = N1-methylpseudouridine
  modified_base 4137: mod_base = OTHER; note = N1-methylpseudouridine
  modified_base 4146: mod_base = OTHER; note = N1-methylpseudouridine
  modified_base 4149: mod_base = OTHER; note = N1-methylpseudouridine
  modified_base 4155: mod_base = OTHER; note = N1-methylpseudouridine
  modified_base 4161: mod_base = OTHER; note = N1-methylpseudouridine
  modified_base 4164: mod_base = OTHER; note = N1-methylpseudouridine
  modified_base 4178: mod_base = OTHER; note = N1-methylpseudouridine
  modified_base 4179: mod_base = OTHER; note = N1-methylpseudouridine
  modified_base 4194: mod_base = OTHER; note = N1-methylpseudouridine
  modified_base 4199: mod_base = OTHER; note = N1-methylpseudouridine
  modified_base 4200: mod_base = OTHER; note = N1-methylpseudouridine
  modified_base 4203: mod_base = OTHER; note = N1-methylpseudouridine
  modified_base 4217: mod_base = OTHER; note = N1-methylpseudouridine
  modified_base 4227: mod_base = OTHER; note = N1-methylpseudouridine
  modified_base 4239: mod_base = OTHER; note = N1-methylpseudouridine
  modified_base 4242: mod_base = OTHER; note = N1-methylpseudouridine
  modified_base 4245: mod_base = OTHER; note = N1-methylpseudouridine
  modified_base 4251: mod_base = OTHER; note = N1-methylpseudouridine
  modified_base 4259: mod_base = OTHER; note = N1-methylpseudouridine
  modified_base 4266: mod_base = OTHER; note = N1-methylpseudouridine
  modified_base 4268: mod_base = OTHER; note = N1-methylpseudouridine
  modified_base 4269: mod_base = OTHER; note = N1-methylpseudouridine
  modified_base 4275: mod_base = OTHER; note = N1-methylpseudouridine
  modified_base 4296: mod_base = OTHER; note = N1-methylpseudouridine
  modified_base 4299: mod_base = OTHER; note = N1-methylpseudouridine
  modified_base 4317: mod_base = OTHER; note = N1-methylpseudouridine
  modified_base 4335: mod_base = OTHER; note = N1-methylpseudouridine
  modified_base 4341: mod_base = OTHER; note = N1-methylpseudouridine
  modified_base 4352: mod_base = OTHER; note = N1-methylpseudouridine
  modified_base 4356: mod_base = OTHER; note = N1-methylpseudouridine
  modified_base 4361: mod_base = OTHER; note = N1-methylpseudouridine
  modified_base 4362: mod_base = OTHER; note = N1-methylpseudouridine
  modified_base 4365: mod_base = OTHER; note = N1-methylpseudouridine
  modified_base 4367: mod_base = OTHER; note = N1-methylpseudouridine
  modified_base 4371: mod_base = OTHER; note = N1-methylpseudouridine
  modified_base 4382: mod_base = OTHER; note = N1-methylpseudouridine
  modified_base 4392: mod_base = OTHER; note = N1-methylpseudouridine
  modified_base 4428: mod_base = OTHER; note = N1-methylpseudouridine
  modified_base 4430: mod_base = OTHER; note = N1-methylpseudouridine
  modified_base 4431: mod_base = OTHER; note = N1-methylpseudouridine
  modified_base 4434: mod_base = OTHER; note = N1-methylpseudouridine
  modified_base 4451: mod_base = OTHER; note = N1-methylpseudouridine
  modified_base 4455: mod_base = OTHER; note = N1-methylpseudouridine
  modified_base 4464: mod_base = OTHER; note = N1-methylpseudouridine
  modified_base 4467: mod_base = OTHER; note = N1-methylpseudouridine
  modified_base 4476: mod_base = OTHER; note = N1-methylpseudouridine
  modified_base 4484: mod_base = OTHER; note = N1-methylpseudouridine
  modified_base 4485: mod_base = OTHER; note = N1-methylpseudouridine
  modified_base 4497: mod_base = OTHER; note = N1-methylpseudouridine
  modified_base 4500: mod_base = OTHER; note = N1-methylpseudouridine
  modified_base 4503: mod_base = OTHER; note = N1-methylpseudouridine
  modified_base 4518: mod_base = OTHER; note = N1-methylpseudouridine
  modified_base 4527: mod_base = OTHER; note = N1-methylpseudouridine
  modified_base 4530: mod_base = OTHER; note = N1-methylpseudouridine
  modified_base 4538: mod_base = OTHER; note = N1-methylpseudouridine
  modified_base 4563: mod_base = OTHER; note = N1-methylpseudouridine
  modified_base 4584: mod_base = OTHER; note = N1-methylpseudouridine
  modified_base 4587: mod_base = OTHER; note = N1-methylpseudouridine
  modified_base 4593: mod_base = OTHER; note = N1-methylpseudouridine
  modified_base 4595: mod_base = OTHER; note = N1-methylpseudouridine
  modified_base 4596: mod_base = OTHER; note = N1-methylpseudouridine
  modified_base 4602: mod_base = OTHER; note = N1-methylpseudouridine
  modified_base 4611: mod_base = OTHER; note = N1-methylpseudouridine
  modified_base 4628: mod_base = OTHER; note = N1-methylpseudouridine
  modified_base 4629: mod_base = OTHER; note = N1-methylpseudouridine
  modified_base 4634: mod_base = OTHER; note = N1-methylpseudouridine
  modified_base 4637: mod_base = OTHER; note = N1-methylpseudouridine
  modified_base 4638: mod_base = OTHER; note = N1-methylpseudouridine
  modified_base 4650: mod_base = OTHER; note = N1-methylpseudouridine
  modified_base 4664: mod_base = OTHER; note = N1-methylpseudouridine
  modified_base 4683: mod_base = OTHER; note = N1-methylpseudouridine
  modified_base 4686: mod_base = OTHER; note = N1-methylpseudouridine
  modified_base 4698: mod_base = OTHER; note = N1-methylpseudouridine
  modified_base 4701: mod_base = OTHER; note = N1-methylpseudouridine
  modified_base 4713: mod_base = OTHER; note = N1-methylpseudouridine
  modified_base 4722: mod_base = OTHER; note = N1-methylpseudouridine
  modified_base 4724: mod_base = OTHER; note = N1-methylpseudouridine
  modified_base 4737: mod_base = OTHER; note = N1-methylpseudouridine
  modified_base 4743: mod_base = OTHER; note = N1-methylpseudouridine
  modified_base 4752: mod_base = OTHER; note = N1-methylpseudouridine
  modified_base 4799: mod_base = OTHER; note = N1-methylpseudouridine
  modified_base 4800: mod_base = OTHER; note = N1-methylpseudouridine
  modified_base 4827: mod_base = OTHER; note = N1-methylpseudouridine
  modified_base 4829: mod_base = OTHER; note = N1-methylpseudouridine
  modified_base 4834: mod_base = OTHER; note = N1-methylpseudouridine
  modified_base 4836: mod_base = OTHER; note = N1-methylpseudouridine
  modified_base 4845: mod_base = OTHER; note = N1-methylpseudouridine
  modified_base 4846: mod_base = OTHER; note = N1-methylpseudouridine
  modified_base 4848: mod_base = OTHER; note = N1-methylpseudouridine
  modified_base 4850: mod_base = OTHER; note = N1-methylpseudouridine
  modified_base 4851: mod_base = OTHER; note = N1-methylpseudouridine
  modified_base 4863: mod_base = OTHER; note = N1-methylpseudouridine
  modified_base 4868: mod_base = OTHER; note = N1-methylpseudouridine
  modified_base 4870: mod_base = OTHER; note = N1-methylpseudouridine
  modified_base 4882: mod_base = OTHER; note = N1-methylpseudouridine
  modified_base 4889: mod_base = OTHER; note = N1-methylpseudouridine
  modified_base 4892: mod_base = OTHER; note = N1-methylpseudouridine
  modified_base 4898: mod_base = OTHER; note = N1-methylpseudouridine
  modified_base 4901: mod_base = OTHER; note = N1-methylpseudouridine
  modified_base 4903: mod_base = OTHER; note = N1-methylpseudouridine
  modified_base 4907: mod_base = OTHER; note = N1-methylpseudouridine
  modified_base 4914: mod_base = OTHER; note = N1-methylpseudouridine
  modified_base 4916: mod_base = OTHER; note = N1-methylpseudouridine
  modified_base 4917: mod_base = OTHER; note = N1-methylpseudouridine
  modified_base 4918: mod_base = OTHER; note = N1-methylpseudouridine
  modified_base 4921: mod_base = OTHER; note = N1-methylpseudouridine
  modified_base 4926: mod_base = OTHER; note = N1-methylpseudouridine
  modified_base 4930: mod_base = OTHER; note = N1-methylpseudouridine
  modified_base 4933: mod_base = OTHER; note = N1-methylpseudouridine
  modified_base 4937: mod_base = OTHER; note = N1-methylpseudouridine
  modified_base 4941: mod_base = OTHER; note = N1-methylpseudouridine
  modified_base 4945: mod_base = OTHER; note = N1-methylpseudouridine
  modified_base 4947: mod_base = OTHER; note = N1-methylpseudouridine
aggactcctccccatcctctccctctgtccctctgtccctctgaccctgcactgtcccagcaccatgagcgaggtggagttcagccacgagtactggatgcgccacgccctgaccctggccaagcgcgcccgcgacgagcgcgaggtgcccgtgggcgccgtgctggtgctgaacaaccgcgtgatcggcgagggctggaaccgcgccatcggcctgcacgaccccaccgcccacgccgagatcatggccctgcgccagggcggcctggtgatgcagaactaccgcctgatcgacgccaccctgtacgtgaccttcgagccctgcgtgatgtgcgccggcgccatgatccacagccgcatcggccgcgtggtgttcggcgtgcgcaacgccaagaccggcgccgccggcagcctgatggacgtgctgcaccaccccggcatgaaccaccgcgtggagatcaccgagggcatcctggccgacgagtgcgccgccctgctgtgccgcttcttccgcatgccccgccgcgtgttcaacgcccagaagaaggcccagagcagcaccgacagcggcggcagcagcggcggcagcagcggcagcgagacacccggcaccagcgagagcgccacccccgagagcagcggcggcagcagcggcggcagcgacaagaagtacagcatcggcctggccatcggcaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagacagccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagacaatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagctgatcaacggcatccgcgacaagcagagcggcaagaccatcctggacttcctcaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacacgccagatcaccaagcacgtggcccagatcctggacagccgcatgaacaccaagtacgacgagaacgacaagctgatccgcgaggtgaaggtgatcaccctcaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagacaaacggcgagacaggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctcaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggcctgtacgagacacgcatcgacctgagccagctgggcggcgacgagggcgccgacaagcgcaccgccgacggcagcgagttcgagagccccaagaagaagcgcaaggtgtaaagtgtccagaccattgtcttccaaccccagctggcctctagaacacccactggccagtcctagagctcctgtccctacccactctttgctacaataaatgctgaatgaatccatctaga